(19) United States
(12) Patent Application Publication
(10) Pub. No.: US 2012/0316218 A1
(43) Pub. Date: Dec. 13, 2012

(54) SMALL NON-CODING REGULARTORY RNA's and METHODS FOR THEIR USE

(76) Inventor: Gennadi V. Glinsky, LaJolla, CA (US)

(21) Appl. No.: 13/261,142

(22) PCT Filed: Jul. 16, 2010
(86) PCT No.: PCT/US10/42346

§ 371 Date: Jan. 13, 2012

Related U.S. Application Data
(63) Continuation-in-part of application No. 61/263,556, filed on Nov. 23, 2009.
(60) Provisional application No. 61/226,448, filed on Jul. 17, 2009.
(60) Provisional application No. 61/263,556, filed on Nov. 23, 2009.
(60) Provisional application No. 61/264,057, filed on Nov. 24, 2009.
(60) Provisional application No. 61/307,666, filed on Feb. 24, 2010.

(51) Int. Cl. C12N 15/113; C12Q 1/68; A61K 31/7088; G01N 33/53; C12N 5/10; C12N 15/85; C40B 30/04

(52) U.S. Cl. 514/44 A; 435/320.1; 536/24.1; 435/6.11; 506/9; 435/7.1; 435/325; 435/366

(57) ABSTRACT

Disclosed are methods and compositions related to small, non-coding RNA molecules having gene regulatory activity, compositions comprising same, and methods for their use. Provided are isolated small non-coding RNA molecules transcribed from an intergenic region of the human genome, wherein the intergenic region contains at least one small nucleotide polymorphism (SNP) associated with one or more human diseases or disorders. Also disclosed are methods for the detection of these small non-coding RNA molecules in a biological sample and related therapeutic, diagnostic, and prognostic methods.

### CROSS REFERENCE TO RELATED APPLICATIONS

This application claims priority to U.S. Provisional Application Nos. 61/226,448, filed Jul. 17, 2009; 61/264,057, filed Nov. 24, 2009; 61/307,666, filed Feb. 24, 2010; and 61/263,556, filed Nov. 23, 2009, each of which is incorporated herein by reference in its entirety.

### INCORPORATION BY REFERENCE OF SEQUENCE LISTING

The contents of the text file named “26141_{—}511001WO_SeqList_ST25.txt” which was created on Jul. 16, 2010 and is 92 KB in size, are hereby incorporated by reference in their entirety.

### FIELD OF THE INVENTION

The invention relates to small, non-coding RNA molecules having gene regulatory activity, compositions comprising same, and methods for their use.

### BACKGROUND OF THE INVENTION

Recent genome-wide analyses of transcription in humans has revealed the surprisingly pervasive transcription of non-coding regions of DNA, both within introns and in intergenic sequences distant from known protein-coding genes. See for review, Malecová and Morris, Curr. Opin. Mol. Ther. 12(2):214-22 (2010). Evidence has emerged of widespread divergent transcription at protein-encoding gene promoters. See Seila, A. C. et al., Science (2008) 322:1849-51. Transcription start site-associated RNAs were found to nonrandomly flank active promoters, with peaks of antisense and sense short RNAs at 250 nucleotides upstream and 50 nucleotides downstream, respectively. These transcription start site RNAs form part of a diverse family of small non-coding RNAs generated from posttranscriptional processing of messenger RNAs. See Fejes-Toth, K. et al., Nature (2009) 457:1028-32. Several kinds of non-coding RNA molecules have been identified that act to regulate gene expression by transcriptional or translational silencing. These are small interfering RNA molecules (“siRNAs”), short hairpin RNA molecules (“shRNAs”), long interfering antisense non-coding RNAs (referred to herein as “liRNAs”), and microRNAs (“miRNAs”).

siRNAs involved in gene silencing have been described in various organisms including S. pombe, T. thermophile, A. thaliana, D. melanogaster and C. elegans. Transcriptional suppression of human genes by exogenously added siRNAs targeted to specific promoters has been well documented. But the mechanism of siRNA action is not well understood. It is believed to involve chromosomal remodeling in the vicinity and downstream of the initial siRNA target site. One type of “remodeling” takes the form of enriching the chromatin at the siRNA-targeted promoter with silent chromatin “marks.” Two of these marks are posttranslational modifications of histone proteins. Specifically, the dimethylation of histone 3 at lysine 9 (“H3K9me2”) and the trimethylation of histone 3 at lysine 27 (“H3K27me3”). The human proteins involved in chromatin remodeling include methyltransferases such as the de novo DNA methyltransferase Dnmt3A, histone deacetylase 1 (“HDAC1”), and the histone lysine methyltransferase KMT6, also known as EZH2.

There is one published case of an exogenously added non-coding RNA molecule mediating long-term transcriptional silencing. This was an shRNA targeted to the promoter of the UBC gene in human cells. UBC gene expression was suppressed for one month even though the shRNA was expressed for only 7 days. The data suggested that the silencing was initially established by histone methylation and followed by DNA methylation. The methylation of CpG islands in the promoter regions of genes is known to play a significant role in the stable, long-term epigenetic silencing of genes throughout development.

liRNAs have been identified in mammalian cells acting to silence particular chromosomal regions, such as the HOX family of genes in eukaryotes and the X chromosome in mice and humans. 231 liRNAs were identified as transcribed from the intergenic regions of the HOX loci. The majority of these were antisense compared to the HOX genes. At least one liRNA was identified (HOTAIR) that negatively regulates a gene (HOXD) distant from its site of transcription. The mechanism apparently involves recruiting proteins of the Polycomb complex to the promoter region and thereby increasing the amount of repressive H3K27me3. The Polycomb (PcG) proteins are transcriptional repressors which act as genome-wide regulators of expression during development. The PcG proteins alter the epigenetic state of chromatin, for example, by increasing histone methylation or ubiquination. It is not clear how the PcG complex is targeted to a specific promoter region, but recruitment of the complex and the subsequent formation of heterochromatin is believed to underlie PcG-mediated gene silencing.

With respect to the X chromosome, an liRNA was identified in humans and mice that mediates silencing. Although the mechanism of action is not known in human cells, in the mouse it appears to involve recruitment of a PcG complex to the promoter region through direct interaction between the liRNA and a subunit of the complex.

liRNAs are also involved in genomic imprinting of autosomal genes. Imprinting is a mono-allelic mechanism of gene silencing based on the parent-of-origin. In at least two cases (Air and Kcnq1ot1) the liRNAs silence large domains of the genome through their interaction with chromatin, specifically be recruiting methyltransferases and PcG complexes to the loci of the silenced genes.

The limited data that exists suggests that non-coding RNA molecules function in combination with PcG proteins and perhaps other, unidentified proteins, to silence the expression of particular genes in cancer cells, such as tumor suppressor genes, analogous to their putative role during development. However, the complex role of these molecules in transcriptional silencing during normal development and in diseases such as cancer remains to be established.

miRNAs are a class of small (20-30 nucleotides in length) non-coding regulatory RNAs that perfectly match the 3′ untranslated regions (3′UTR) of target messenger RNAs. Binding of the miRNA to its target sequence results in degradation of the messenger RNA or inhibition of its translation. See for review, He, L. and Hannon, G. J. Nat. Rev. Genet. (2004) 5:522-531.

Large-scale genome-wide associations studies (GWAS) of small nucleotide polymorphisms (SNPs) have identified genetic variants associated with disease phenotypes at high levels of statistical confidence. The dominant approach to understanding how these genetic variations contribute to disease has been to examine the effects of the SNP allelic variants on nearby protein-coding genes. This protein-centric strategy was recently extended to the SNPs residing within the boundaries of genomic regions encoding microRNAs (miRNAs) and also within miRNA target sites in messenger RNAs.

The present inventors demonstrated that many disease-linked SNPs are located far from protein-coding genes but in transcriptionally active regions of the genome. The invention is based upon the discovery of a novel class of non-coding RNAs transcribed from these intergenic regions containing disease-linked SNPs.

### SUMMARY OF THE INVENTION

The present invention is based upon the discovery that genomic regions containing disease-associated small nucleotide polymorphisms (SNPs) are actively transcribed to produce small non-coding SNP-bearing RNA molecules having biological activity. These RNA molecules are referred to herein as “snpRNAs”. The small non-coding SNP-bearing RNA molecules of the invention have biological activity. In particular, specific RNA molecules of the invention are demonstrated to modulate the expression of other non-coding RNA molecules as well as protein-coding genes. In one embodiment, the small non-coding SNP-bearing RNA molecules of the invention modulate the activity of the innate immunity/inflammasome pathway by modulating the expression of particular genes in that pathway. In a specific embodiment, an snpRNA molecule of the invention modulates the expression of a gene selected from NLRP3, NLRP1, HMGA1, and MYB. In another embodiment, an snpRNA molecule of the invention facilitates hormone-independent growth of a hormone-dependent cell or cell line. In a specific embodiment, the hormone-dependent cell is a prostate cell. In one embodiment, the cell is a prostate cancer cell.

In certain embodiments, the snpRNAs regulate the expression of genes distant from their site of transcription, and thus may also be referred to as “transRNAs.” The invention provides the sequences of specific cDNA molecules corresponding to the snpRNAs described herein, methods and reagents for their detection in a biological sample from a subject, and methods for their use in diagnostic and prognostic assays.

An snpRNA molecule of the invention contains a disease-associated SNP which is located within a loop structure of the RNA molecule. Preferably, this loop structure containing the SNP also contains a binding site for a microRNA (“miRNA”) molecule. Preferably, the SNP is located within a binding site for one or more of the following proteins: H3K27Me3, CBP/CREB, Ezh2, and POL2. In certain embodiments where the SNP is located within the binding site for more than one protein, the binding sites overlap. In another embodiment, the SNP is within the binding site for a nuclear lamina protein. In a specific embodiment, the SNP is located within 200 basepairs of a binding site for a lamin B1 protein.

In one embodiment, the invention provides isolated, purified cDNA molecules corresponding to the snpRNA molecules described herein. The cDNA molecules are useful to express the snpRNA molecules of the invention in heterologous cells and to detect the presence of the snpRNA molecules in a biological sample from a subject. In certain embodiments, the cDNA molecules are useful as probes to detect the snpRNA molecules in the sample, e.g., in hybridization based assays. In other embodiments the cDNA molecules are used as positive controls for the detection of the snpRNA molecules in a biological sample from a subject.

The invention provides an isolated small non-coding RNA molecule transcribed from an intergenic region of the human genome, wherein the RNA molecule is less than 500, less than 400, less than 300, less than 200, less than 150, less than 100, or less than 75 nucleotides and the intergenic region contains at least one small nucleotide polymorphism (SNP) associated with one or more human diseases or disorders. In a particular embodiment, the intergenic region contains only one SNP. In one embodiment, the snpRNA molecule is contiguous.

In one embodiment, the cDNA form of the RNA molecule comprises a sequence selected from the group consisting of SEQ ID NOs: 1-101, 332, and 333. In another embodiment, the cDNA form of the RNA molecule comprises a sequence selected from the group consisting of SEQ ID NOs: 1, 4, 6, 7, 9-18, 39, 88-90, 332, and 333. In another embodiment, the cDNA form of the RNA molecule comprises a sequence selected from the group consisting of SEQ ID NOs: 1, 7, 332, and 333.

In one embodiment, the SNP is selected from the group consisting of rs2670660, rs6596075, rs6983561, rs16901979, rs13281615, rs10505477, rs10808556, rs6983267, rs7014346, rs7000448, rs1447295, rs2820037, rs889312, rs1937506, rs13387042, rs7716600, rs11249433, and rs3803662.

In one embodiment, the SNP is selected from the group consisting of, rs9469220, rs9270986, rs6457617, rs615672, rs7837688, rs6997709, rs16892766, rs2670660, and rs2542151.

The invention also provides a vector comprising a polynucleotide encoding an RNA molecule of the invention. In one embodiment, the vector comprises the cDNA form of an RNA molecule described herein. The invention further provides a cell comprising said vector. In one embodiment, the cell is ex vivo or in vitro.

The invention also provides a kit comprising, in one or more containers, a vector comprising a polynucleotide encoding an RNA molecule of the invention. In one embodiment, the vector comprises the cDNA form of an RNA molecule described herein and instructions for expressing the RNA molecule from the vector. In one embodiment, the kit further comprises one or more polynucleotide primers for amplifying an RNA or a cDNA molecule of the invention. In one embodiment, the one or more primers comprises a sequence selected from the group consisting of SEQ ID NOs: 102-331. In one embodiment, the one or more primers comprises a sequence selected from the group consisting of SEQ ID NOs: 102-161. In one embodiment, the one or more primers comprises a sequence selected from the group consisting of SEQ ID NOs: 102, 103, 114, 115, 326, and 327.

The invention also provides a kit comprising, in one or more containers, a cell comprising said vector and instructions for expressing the RNA molecule in the cell.

The invention also provides a method for detecting the small non-coding RNA molecules described herein in a sample from a subject, the method comprising detecting the RNA molecules in the sample. In one embodiment, step of detecting the RNA molecules comprises the step of detecting the cDNA form of the RNA molecule in the sample. In one embodiment, the cDNA form is detected by a method comprising reverse transcription and polymerase chain reaction (RT-PCR) technology. In another embodiment, the cDNA form is detected by a method comprising nucleic acid hybridization technology.

In one embodiment, the method further comprises the steps of isolating the small RNA fraction from the sample and converting the RNA into cDNA prior to the step of detecting the cDNA in the sample.

The invention also provides a method for evaluating the risk that a human subject will develop a disease or condition associated with a specific allele of an SNP (“the pathological allele”) by detecting the presence of an RNA molecule of claim 1 in a sample from the subject, wherein the RNA molecule is transcribed from the pathological allele, and wherein detection of said RNA molecule indicates that the subject has an increased risk for developing the disease or condition and the failure to detect said RNA molecule indicates that the subject has a decreased risk for developing the disease or condition.

In one embodiment, the method further comprises detecting the expression level of the RNA molecule transcribed from the pathological allele relative to its expression in a population of healthy subjects, wherein an increased or decreased level of expression relative to the population of healthy subjects indicates that the subject has an increased risk for developing the disease or condition.

In one embodiment, the step of detecting the presence of an RNA molecule transcribed from the pathological allele is performed indirectly, by detecting the expression of one or more genes whose expression is regulated by the RNA molecule.

The invention also provides a method for diagnosing a disease or condition associated with a specific allele of an SNP (“the pathological allele”) in a human subject, the method comprising detecting the presence of an RNA molecule of claim 1 in a sample from the subject, wherein the RNA molecule is transcribed from the pathological allele, and wherein the disease or condition is positively diagnosed if the RNA molecule is detected in the sample.

The invention also provides a method for treating, preventing, or ameliorating a disease or condition associated with a specific allele of an SNP (“the pathological allele”) in a subject in need thereof, the method comprising administering one or more therapeutic agents that act to suppress the expression or antagonize the activity of an RNA molecule of claim 1, wherein the RNA molecule is transcribed from the pathological allele.

In one embodiment of the claimed methods, the presence of the G-allele snpRNA of rs2670660 is detected in a sample from the subject, wherein the presence of the G-allele snpRNA indicates that the subject is at an increased risk for developing a disease or disorder selected from vitiligo, Crohn's disease, rheumatoid arthritis, Huntington's disease, Alzheimer's disease, breast cancer, metastatic breast cancer, prostate cancer, metastatic prostate cancer, autism, and obesity.

In one embodiment of the claimed methods, the presence of the A-allele snpRNA of rs16901979 is detected in a sample from the subject, wherein the presence of the A-allele snpRNA indicates that the subject is at an increased risk for developing a cancer of epithelia origin. In one embodiment, the cancer is selected from breast cancer, metastatic breast cancer, prostate cancer, and metastatic prostate cancer.

Preferably, with respect to any of the methods described above, the subject is human.

In certain embodiments of the methods described above, the sample is a blood, tissue, or cell sample.

In one embodiment, the disease or condition is selected from the group consisting of vitiligo, Crohn's disease, rheumatoid arthritis, Huntington's disease, Alzheimer's disease, breast cancer, metastatic breast cancer, prostate cancer, metastatic prostate cancer, autism, and obesity.

In one embodiment, the disease or condition is selected from the group consisting of autism, alzheimer's disease, schizophrenia and bipolar disorder.

In one embodiment, the disease or condition is an autoimmune disease or disorder. In one embodiment, the disease or condition is selected from the group consisting of vitiligo, ankylosing spondylitis, rheumatoid arthritis, multiple sclerosis, systemic lupus erythematosus and autoimmune thyroid disease.

In one embodiment, the disease or condition is selected from the group consisting of ulcerative colitis and Crohn's disease.

In one embodiment, the disease or condition is selected from the group consisting of breast cancer, colorectal cancer, lung cancer, ovarian cancer, and prostate cancer.

In one embodiment, the disease or condition is selected from the group consisting of coronary artery disease, hypertension, type 1 diabetes, type 2 diabetes, and obesity.

The invention also provides an apparatus for evaluating a disease or condition, or evaluating the risk of developing a disease or condition, in a subject, the apparatus comprising a model configured to evaluate a dataset for the subject to thereby evaluate the risk of disease in the subject, wherein the model is based upon determining the similarity in the expression profile of a defined set of genes in a sample from the subject and the expression profile for that set of genes in one or more reference sets of the model, wherein a reference set comprises one or more of a population of healthy subjects and a population of subjects suffering from the disease, wherein the set of genes is a set of genes whose expression is regulated by a small RNA molecule of claim 1.

### BRIEF DESCRIPTION OF THE FIGURES

FIG. 1: Identification of 12 small RNAs encoded by intergenic disease-associated SNPs using reverse-transcription PCR-based screening. Small RNA fractions were isolated from various human cell lines and subjected to the RT-PCR based screen. PCR products of expected size were purified, subjected to the nested PCR analysis and gel electrophoresis. Molecular identities of identified RNA molecules were validated by sequencing of primary PCR and nested PCR products. The 12 RNAs identified by this method are designated A3, A6, A9, A16, A21-26, A28, and A29. The sequences are given in Table 1. The primers used to amplify the sequences are given in Table 3. FIG. 15 shows the identification of other RNAs from the “A” set in different cell lines.

FIG. 2: (A) Genomic coordinates of the endogenous small RNAs described in FIG. 1 and corresponding disease-associated SNPs. Abbreviations used: Crohn's disease (CD), rheumatoid arthritis (RA), type 1 diabetes (T1D), autoimmune disorders (AID), hypertension (HT), prostate cancer (PC), breast cancer (BC), ovarian cancer (OC), colorectal cancer (CRC).

(B): Examples of predicted secondary structures of RNAs. Arrows indicate the positions of nucleotides variations which are associated with increased risk of developing corresponding disorders. Bottom right panel shows alignments of the miRNA target sites in RNA A21, which is transcribed from a region containing the prostate cancer susceptibility SNP rs7837688. Individual human miRNAs (short horizontal bars) are aligned along the A21 RNA sequence according to the positions of respective target sites. Single vertical bar marks the position of the prostate cancer-predisposition SNP. Note that a vast majority of microRNA target sites segregates to the A21 transRNA segment around the SNP and includes SNP nucleotides.

(C) Chromatin state map analysis of genomic sequences encoding evolutionary conserved snpRNAs reveals a consensus chromatin domain signature comprising histone H3K27Me3, CBP/CREB, EZH2, and POL2 proteins. Chromatin state maps of corresponding human and mouse genome sequences are visualized using the custom tracks of the UCSC Genome Browser. Color-coded horizontal lines depict alignments of DNA sequences derived from Chip-Seq experiments using antibodies against corresponding proteins. Each color-coded horizontal line represents data from independent biological replicates. Note nearly ubiquitous alignments of the evolutionary-conserved RNA-encoding sequences within binding sites of the histone H3K27Me3, CBP/CREB, EZH2, and POL2 proteins. Positions of disease-linked SNP nucleotides within RNA-encoding sequences are indicated by arrows and vertical lines. Original experiments describing the corresponding mouse and human genome-wide chromatin state maps were reported elsewhere

FIG. 3: Identification of rs2670660-encoded endogenous transRNAs.

(A) Sequence mapping of nucleotide primer sets utilized for identification of rs2670660-encoded endogenous small RNAs and corresponding PCR products. Sense and anti-sense variants of a 52 nucleotide (“nt”) rs2670660 sequence (shown in a shaded box, SEQ ID NO:1) were chemically synthesized, cloned into GFP-expressing lentiviral vectors, and utilized in biological and mechanistic experiments.

(B) PCR analysis of genomic DNA products generated by individual sets of primers shown in (A).

(C) PCR analysis of cDNA products derived from small RNA fraction <200 nt using primer sequences shown in (A). Only primer set 2 generated a product of the expected size (152 nt).

(D-F) Nested PCR using primer sets 1 and 2 in the small RNA fraction from BJ1 cells. Products of the expected size for set 2 (152) and set 1 (110 nt) are shown. Sequences of PCR products were confirmed by direct sequencing. Nested PCR of the 152 nt product with primer set 1 using small RNA fractions (containing RNA of less than 200 nt in length) from various cell lines as template. Product of the expected size (110 nt) is shown. Sequences of PCR products were confirmed by direct sequencing.

(G) Sequence homology profiling of rs2670660-encoded RNAs, miRNAs, and long non-coding RNAs identifies extensive sequence homology/complementarity features.

a) Genomic location (top left), secondary structures of 152 nt (bottom left) and 52 nt (top right) RNA molecules, and position of the miRNA-target sites along the 152 nt transRNA sequence (bottom right).

Visualization of individual miRNA-target sites within the rs2670660-encoded RNA.

c, d) miRNAs which are differentially regulated in BJ1 cells expressing distinct allelic variants of the NALP1-locus transRNAs share multiple sequence identity segments of at least 11 nucleotides in length with sequences of MEG3 (c) and MALAT1 (d) long non-coding RNAs.

FIG. 4: Expression of a small RNA transcribed from the G-allele of rs2670660 inhibits cell growth and results in G1 arrest. The following notation is used to designate the 4 small RNAs transcribed from the A-allele, the G-allele, and their antisense counterparts: A, G, asA, and asG. These 4 RNAs are also referred to collectively as “the '660 RNAs.” Transfected BJI cells were sorted by GFP expression and an enriched population (>90% GFP positive) was used in monolayer and clonal growth assays.

(A) Monolayer cultures expressing GFP only (BJI/GFP), or 50 nucleotide RNAs from the G-allele (rs2670660_G) or the A-allele (rs2670660_A) of the SNP rs2670660 were cultured for five days; cells were counted every 24 hours. Top line in graph is A; middle line is GFP only; bottom line is G.

(B) Clonal growth of cells expressing GFP only (EGFP), the G-allele RNA (1), the A-allele RNA (2), the anti-sense G allele RNA (3), or the anti-sense A-allele (4). Cells were cultured as described in methods. The average of triplicates is shown.

(C) Flow cytometric analysis (FACs) of cells expressing empty vector (GFP), sense and anti-sense (as) variants of the A- and G-allele RNAs. Representative FACs plots are shown above the bar graphs which represent the number of cells in each phase of the cell cycle (G1, S, G2M), normalized to the vector control. Average values of three independent biological replicates are shown.

FIG. 5: Representative results of clonogenic growth experiments of BJ1 cells expressing sense and anti-sense allele small RNAs encoded by rs2670660.

(A): cells expressing GFP from vector controls lacking insert (GFP, top row), or one of the following small RNAs encoded by rs2670660 (next 4 rows): A-allele (A), G-allele (G), anti-sense A (asA), or anti-sense G (asG).

(B): top to bottom rows show cells co-expressing the following transcripts: G and vector control (GFP); asG; asA and vector control; A and asA; vector control alone; G and asA.

FIG. 6: Constitutive expression of distinct allelic variants of NALP1-locus transRNAs exerts allele-specific effects on phenotypes of human cells.

(A) Expression of the G-allele of the rs2670660-encoded RNA interferes with TPA-induced monocyte/macrophage differentiation. THP-1 cells expressing control vector or allele-specific sense and anti-sense variants of rs2670660-encoded RNAs were treated with TPA for 4 days to induce differentiation into macrophages. Left panels (top to bottom) show light microscopy images of control, A-allele, and G-allele transfected cells. Right panels show fluorescence images of the same. The cells expressing the G-allele variant failed to differentiate and retained a non-differentiated state.

(B) In response to induction of differentiation, THP-1 cells expressing the G-allele of the rs2670660-encoded RNA undergo massive apoptosis and produce ˜5-fold less macrophages which are twice less potent in the sheep erythrocyte phagocytosis assay compared to macrophages derived from THP-1 cells expressing A-allele RNAs.

(C) Human cells stably expressing G-allele RNAs manifest diminished expression levels of the genes comprising PRC1-type Polycomb group (PcG) proteins chromatin remodeling complexes (BMI1 and RING1B) compared to components of the PRC2-type PcG proteins chromatin silencing complexes (EZH2, EED, SUZ12) and differential regulation of the 586 transcripts encoded by PcG pathway-targets, bivalent chromatin domain genes.

(D) Allele-specific effects on monocyte/macrophage differentiation are modulated by BMI1 expression. BMI1 knock-down markedly diminishes macrophage production by A-allele expressing THP-1 cells (top and bottom left panels), whereas BMI1 over-expression rescues the macrophage-producing defect of G-allele expressing THP-1 cells (bottom right panels). Inserts show the results of RT-PCR analysis validating the efficiency of the gene knock-down (insert, bottom left panels) and gene transfer (inset, bottom left panels) experiments.

(E) G-allele expressing human fibroblast BJ1 cells manifest significantly higher motility compared to ancestral A-allele expressing BJ1 cells. Gaps of defined distances were created in confluent cultures of BJ1 cells and motility sequences were continuously monitored and recorded using time-lapse video cinematography. For each culture, the initial distance, motility sequence time (time to complete closing of the gap), and motility speed were measured. Average values of six replicate measurements are reported.

FIG. 7: Gene expression patterns of BJI cells expressing allele-specific RNAs encoded by the rs2670660 sequence. Gene expression was analyzed using Affymetrix HG-U133A Pus 2.0 microarrays. Panels A-D each show two (A, C) or three (B, D) rows of paired bars representing the expression of representative genes in cells expressing, from left to right, G, A, asG, asA, or GFP only (unlabeled, 5^{th} set of bars for each gene). Panel A shows the expression data for 4 particular genes, Panel B for 9 genes, Panel C for 4 genes, and Panel D for 9 genes. Panels E-M show the same relationships for large sets of genes using linear regression analysis to demonstrate the concordant and discordant patterns of gene expression under the various allele-specific conditions. In panels E-M, the y-axis is mRNA expression and the x-axis represents individual genes. Thus, each dot on the graph represents the mRNA expression level of a particular gene.

(A, B): examples of allele specific antagonism of gene expression for genes showing increased expression in BJ1 cells in response to ectopic expression of the G-allele RNA and decreased expression in response to ectopic expression of the A-allele RNA of rs2670660.

(C, D): Examples of allele specific antagonism of gene expression for genes showing decreased expression in BJ1 cells in response to ectopic expression of the G-allele RNA and increased expression in response to ectopic expression of the A-allele RNA of rs2670660.

(E, F): A set of 3299 genes whose expression was differentially regulated in cells expressing the G-allele RNA of rs2670660 compared to vector controls was defined by t-statistics. The expression of these 3299 genes was then evaluated in cells expressing the G-allele RNA and in cells expressing the A-allele RNA of rs2670660. Regression analysis shows highly concordant expression of this set of genes in cells expressing the G- and A-allele RNA of rs2670660.87% of the 3299 genes were concordantly expressed (1562 up- and 1732 down-regulated) (Panel E). Concordance was greater 95% for a subset of 1491 genes identified as differentially expressed in cells expressing the G-allele RNA of rs2670660 (at p=0.05) and then evaluated in in cells expressing the G-allele RNA and in cells expressing the A-allele RNA of rs2670660 (at p=0.1) (Panel F).

(G, H): A set of 3268 genes whose expression was differentially regulated in cells expressing the G-allele compared to cells expressing the A-allele RNA of rs2670660 was defined by t-statistics. The expression of these 3268 genes was then evaluated in cells expressing the G-allele of rs2670660 compared to vector controls. Regression analysis shows highly concordant expression of this set of genes. 89% of 3268 genes were concordantly expressed (1583 up- and 1685 down-regulated). Concordance was greater than 95% for a subset of 1568 genes identified as differentially expressed in cells expressing the G-allele RNA of rs2670660 (at p=0.05) and then evaluated in cells expressing the G-allele RNA and in cells expressing vector controls (at p=0.1) (Panel H).

(I-L): The set of 3299 genes whose expression was differentially regulated in cells expressing the G-allele RNA of rs2670660 compared to vector controls was evaluated in cells expressing the G-allele RNA and in cells expressing the A-allele RNA of rs2670660. Panel I (top) shows the discordant expression of these genes (A- versus G-). The lower panel shows the discordant expression of a subset of 418 genes whose expression was differentially regulated by at least 4-fold.

(J): 2598 genes were identified as differentially regulated by t-statistics in A-allele small RNA-expressing cells compared to the control cultures. Panel J (top) shows the discordant expression profile for these genes in G-allele RNA-expressing cells compared to A-allele RNA-expressing cells. The lower panel shows the discordant expression of a subset of 379 genes whose expression was differentially regulated by at least 4-fold.

(K): 2844 genes were identified as differentially regulated by t-statistics in asG-allele small RNA-expressing cells compared to the control cultures. Panel K (top) shows the discordant expression profile for these genes in asA-allele RNA-expressing cells compared to asG-allele RNA-expressing cells. The lower panel shows the discordant expression of a subset of 352 genes whose expression was differentially regulated by at least 4-fold.

(L): 2766 genes were identified as differentially regulated by t-statistics in asA-allele small RNA-expressing cells compared to the control cultures. Panel K (top) shows the discordant expression profile for these genes in asG-allele RNA-expressing cells compared to asA-allele RNA-expressing cells. The lower panel shows the discordant expression of a subset of 342 genes whose expression was differentially regulated by at least 4-fold.

FIG. 8: Expression of rs2670660-encoded allele-specific variants of small RNAs induces mRNA expression changes of the inflammasome regulatory genes (NLRP1, NLRP3, HMGA1, Myb).

(A) mRNA expression changes of the NLRP1 (top left panel) and HMGA1 (top right panel) genes in BJ1 cells expressing the A- or G-alleles of the rs2670660-encoded RNAs. Bottom panels show the ratios of NLRP3 to NLRP1 (bottom left) and HMGA1 to Myb (bottom right).

(B) mRNA expression of the NLRP1 and NLRP3 genes in circulating human neutrophils (left panels) and alveolar neutrophils (right panels) after bronchoscopic endotoxin (LPS) challenge. Top panels show NLRP1 and NLRP3 expression. Bottom panels show the ratio of NLRP3 to NLRP1 expression.

(C) mRNA expression changes of the NLRP1 and NLRP3 genes in human leukocytes after in vitro LPS challenge. Left panels (top and bottom) show the expression in unstimulated cells. Right panels show expression in LPS-stimulated cells. Bottom panels show NLRP3/NLRP1 expression ratios in unstimulated (bottom left) and LPS-stimulated cells (bottom right).

(D) mRNA expression changes of the HMGA1 and Myb genes in human circulating human neutrophils (left panels) and alveolar neutrophils (right panels) after bronchoscopic endotoxin (LPS) challenge. Top panels show HMGA1 and Myb expression. Bottom panels show the ratio of HMGA1 to Myb expression.

(E) mRNA expression changes of the HMGA1 (top left) and Myb (top right) genes in human monocytes undergoing adhesion-induced transdifferentiation. Bottom panels show HMGA1/Myb mRNA expression ratios in non-adherent cultures (bottom left) and differentiating cultures (bottom right).

FIG. 9: Microarray analysis of gene expression signatures (GES) associated with expression of rs2670660-encoded small RNAs identifies human cells with experimentally-induced activation of the inflammasome pathway. Expression profiles of G-allele concordant and G-allele discordant signatures in individual experimental and control samples of each data set were evaluated by calculating Pearson correlation coefficients (signature scores) using log 10-transformed fold expression changes of G-allele-specific GES in BJ1 cells as a multidimensional standard vector. Shaded area identifies the range defined by the average +1-2STDEV values of the signature scores in control set of samples.

(A) Expression profiles (bars) and linear regression analysis (scatter) of an 82 gene G-allele concordant signature in human circulating leukocytes after in vitro endotoxin (LPS) challenge. Note distinct expression profiles of G-allele concordant signatures in experimental (left set of bars) and control (right set of bars) samples.

(B) Expression profiles (bars) and linear regression analysis (scatter) of a 262 gene G-allele concordant signature in human alveolar (left set of bars) and circulating (right set of bars) neutrophils after in vivo bronchoscopic endotoxin (LPS) challenge. Note distinct expression profiles of G-allele concordant signatures in alveolar (left set of bars) and circulating (right set of bars) neutrophils.

(C) Expression profiles (bars) and linear regression analysis (scatter) of a 43 gene G-allele concordant signature in human circulating neutrophils after in vivo bronchoscopic endotoxin (LPS) challenge. Note distinct expression profiles of G-allele concordant signatures in circulating neutrophils from LPS-exposed subjects (left set of bars) and control subjects (right set of bars).

(D) Expression profiles (bars) and linear regression analysis (scatter) of a 134 gene G-allele discordant signature in human circulating leukocytes after in vitro endotoxin (LPS) challenge. Note distinct expression profiles of G-allele concordant 45 signatures in experimental (left set of bars) and control (right set of bars) samples.

(E) Expression profiles (bars) and linear regression analysis (scatter) of a 325 gene G-allele discordant signature in human alveolar (left set of bars) and circulating (right of bars) neutrophils after in vivo bronchoscopic endotoxin (LPS) challenge. distinct expression profiles of G-allele concordant signatures in alveolar (left of bars) and circulating (right set of bars) neutrophils.

(F) Expression profiles (bars) and linear regression analysis (scatter) of a 51 gene G-allele concordant signature in human circulating neutrophils after in vivo bronchoscopic endotoxin (LPS) challenge. Note distinct expression profiles allele concordant signatures in circulating neutrophils from LPS-exposed subjects (left set of bars) and control subjects (right set of bars).

(G) Diminished sample discrimination by GES associated with expression of G-specific 52 nt small RNAs without segregation into concordant and discordant subsets. Designations of control and experimental samples as in A-F. From left to right, the number of genes in each signature is 216, 587, and 94.

FIG. 10: microRNA-signatures induced by expression of rs2670660-encoded transRNAs and associated mRNA GES recapitulating miRNA expression patterns. miRNAs differentially-regulated by rs2670660-allele-specific sense and anti-sense 52 nt small RNAs in BJ1 cells were identified using the quantitative PCR protocol for detection of 365 human miRNAs in a 384-well-format TaqMan Low Density Arrays (TaqMan Human MicroRNA Array v1.0; Applied Biosystems). Expression of selected differentially-regulated microRNAs (miR-20b and miR-375) and control miRNAs (miR-205) was induced in BJ1 cells by lentiviral gene transfer and resulting cell lines were subjected to microarray analysis using Affymetrix HG-U133 Plus 2.0 chips.

(A) Expression profiles (bars) and linear regression analysis of expression patterns (scatter) of the 47 miRNA-signature manifesting highly concordant patterns of expression induced by all four allelic variants of the rs2670660 RNAs.

(B) Expression profiles defined by the RQ values (left) and log 10-ransformed RQ values of the 38 miRNA-signature manifesting highly allele-specific patterns of expression induced by distinct sense and anti-sense allelic variants of the rs2670660 RNAs. Note that expression of each miRNA is below Q-PCR detection limit in at least one cell variant and markedly up-regulated (8.4-fold to 496.3-fold) in at least one cell variant.

(C) Expression profiles (bars) and linear regression analysis of expression patterns (scatter) of the 140-gene mRNA-signature manifesting highly concordant patterns of expression induced by all four allelic variants of the rs2670660 RNAs.

(D) Expression profiles of the 59-gene mRNA-signature defined by expression of the miR-20b microRNA in BJ1 cells and manifesting highly concordant patterns of expression induced by all four allelic variants of the rs2670660 RNAs.

(E,F) Expression profiles (bars) and linear regression analysis of expression patterns (scatter) of the 86-gene mRNA-signature which was selected to resemble allele-specific patterns of expression of miR-375 (bottom left set of bars). Note that expression profile of 14-gene mRNA-signature (bottom right sets of bars), which was independently defined by induced expression of miR-375 in BJ1 cells, recapitulates G/A-allele-antagonistic patterns of expression of the 86-gene mRNA-signature and miR-375 microRNA. mRNAs comprising the 14-gene signature are sub-set of mRNAs comprising the 86-gene signature.

(G) Linear regression analysis of microRNA expression patterns exhibiting concordant (top two scatter plots) and discordant (bottom two scatter plots) allelic context-defined expression profiles induced by expression of the rs2670660-encoded 52 nt transRNAs (top left, G and asA alleles; top right, A and asG alleles; bottom left, A and asA alleles; bottom right, G and asG alleles).

(H) Microarray analysis of human BJ1 cells stably expressing distinct allelic variants of the rs2670660-encoded snpRNAs reveals allele-specific alterations of expression in multiple classes of non-coding RNAs including snoRNAs and snoRNA-host genes (SNORD113; SNHG1; SNHG3; SNHG8), long non-coding RNAs (MEG3, tncRNA, and MALAT1), miRNAs, miRNA-precursors, and protein-coding miRNA-host genes (ATAD2; KIAA1199).

(I) An ABI PCR-based screen identified a statistically significant set of 36 microRNAs expression of which is altered at least 1.5-fold in NALP1-locus snpRNA-expressing cells compared to control BJ1/EGFP cells and differentially regulated in pathology-linked G-allele-expressing BJ1 cells compared to the ancestral A-allele-expressing cells.

(J) Allele affinity model of snpRNA-mediated regulation of miRNA expression and activity.

(a)-(c): high affinity (low mfe) snpRNA alleles facilitate increase abundance levels of corresponding miRNAs. Inverse correlation between allele-specific changes in minimal free energy (mfe) of snpRNA/miRNA hybridization and experimentally-defined changes of miRNA expression and activity that is lower mfe values correspond to higher levels of miRNA expression and activity. These relationships are shown for miRNAs the abundance levels of which in human cells are induced (miR-302a; miR-629; miR-548d; miR-200a; miR-627; miR-770-5p) or repressed (miR-133a; miR-20b; miR-205; let-7b) by forced expression of pathology-linked G-allele snpRNAs compared to ancestral A-allele-expressing cells. Insert bars show the results Q-PCR analysis of expression of corresponding microRNAs.

(d) Luciferase reporter assay of miR-205 and let-7b activities in RWPE1 cells stably expressing distinct allelic variants of the NALP1-locus transRNAs demonstrates increased activity of both microRNAs in high affinity ancestral A-allele-expressing cells compared to low affinity pathology-linked G-allele-expressing cells.

(e) Application of the allele affinity model of transRNA-mediated regulation of microRNA expression and activity to development of the allele equilibrium hypothesis explaining the phenotype-altering effects of transRNAs as the consequence of direct actions on microRNAs abundance and activity and down-stream effects of transRNA-regulated microRNAs on expression of protein-coding genes.

FIG. 11: rs2670660-encoded RNAs alter expression of the PluriNet network transcripts and Polycomb pathway genes. Gene expression signatures (GES) associated with expression of rs2670660-encoded sense and anti-sense allele-specific 52 nt small RNAs in BJ1 cells were independently identified for each experimental setting using t-statistics and 155 differentially-regulated transcripts of the PluriNet network and Polycomb pathway were selected for visualization.

(A) Expression profiles (bars) and linear regression analysis of expression patterns (scatters) of PluriNet network transcripts defined as differentially regulated by the indicated allele-specific variants of the rs2670660-encoded transRNAs: the G-allele signature of 100 PluriNet genes; the A-allele signature of 28 PluriNet genes; the asA-allele signature of 77 PluriNet genes; and the asG signature of 42 PluriNet genes.

Note highly concordant expression profiles for G and asA (top left); A and asG (top right); asA and G (bottom left); asG and A (bottom right) signatures. Middle panel shows integrated allele-context-defined views of expression profiles of 155 PluriNet network transcripts expression of which is altered by rs2670660-encoded small RNAs. Note that almost all PluriNet transcripts expression of which is altered by G and asA allele-specific rs26700660 transRNAs are upregulated suggesting that expression of G-allele-specific transRNAs would favor retention of a less-differentiated state in a cell.

(B) G-allele-specific rs2670660-encoded transRNAs induce concomitant upregualtion of the Polycomb Repressive Complex 2 (PRC2) genes Ezh2, Suzl2, and EED. Individual measurements of the mRNA expression levels of corresponding genes derived from two independent biological replicate experiments are shown. Note that in contrast to the PRC2 genes, the expression level of the BMI1 gene, a key component of the PRC1 complex, is decreased in BJ1 cells expressing G-allele-specific rs2670660-encoded transRNAs compared to A-allele-specific transRNA-expressing cells.

FIG. 12: Microarray analysis of gene expression signatures (GES) associated with expression of rs2670660-encoded small RNAs discriminates peripheral blood mononuclear cells (PBMC) from patients with multiple common human disorders and control subjects. GES associated with expression of G-allele-specific 52 nt small RNAs in BJ1 cells was identified using t-statistics and screened for concordant and discordant features in corresponding clinical settings to segregate G-allele concordant and G-allele discordant signatures. Expression profiles of G-allele concordant and G-allele discordant signatures in individual samples of each data set were evaluated by calculating Pearson correlation coefficients (signature scores) using log 10-transformed fold expression changes of G-allele-specific GES in BJ1 cells as a multidimensional standard vector. Shaded area identifies the range defined by the average +/−2STDEV values of the signature scores in control set of samples.

(A) Expression profiles (bars) and linear regression analysis (scatter) of a 309 gene G-allele concordant signature in PBMC of patients with Crohn's disease (left set of bars), ulcerative colitis (right set of bars), and control subjects (middle set of bars). Note distinct expression profiles of G-allele concordant signatures in PBMC from patients and control individuals.

(B) Expression profiles (bars) and linear regression analysis (scatter) of a 203 gene G-allele concordant signature in PBMC of patients with rheumatoid arthritis (left set of bars) and control subjects (middle set of bars). Note distinct expression profiles of G-allele concordant signatures in PBMC from patients and control individuals.

(C) Expression profiles (bars) and linear regression analysis (scatter) of a 525 gene G-allele concordant signature in PBMC of patients with symptomatic Huntington's disease (left set of bars), asymptomatic Huntington's disease (middle set of bars), and control subjects (right set of bars). Note distinct expression profiles of G-allele concordant signatures in PBMC from patients and control individuals.

(D) Expression profiles (bars) and linear regression analysis (scatter) of a 25 gene G-allele concordant signature in PBMC of patients with Alzheimer's disease (left set of bars) and control subjects (middle set of bars). Note distinct expression profiles of G-allele concordant signatures in PBMC from patients and control individuals.

(E) Expression profiles (bars) and linear regression analysis (scatter) of a 439 gene G-allele discordant signature in PBMC of patients with Crohn's disease (left set of bars), ulcerative colitis (right set of bars), and control subjects (middle set of bars). Note distinct expression profiles of G-allele concordant signatures in PBMC from patients and control individuals.

(F) Expression profiles (bars) and linear regression analysis (scatter) of a 190 gene G-allele discordant signature in PBMC of patients with rheumatoid arthritis (left set of bars) and control subjects (middle set of bars). Note distinct expression profiles of G-allele concordant signatures in PBMC from patients and control individuals.

(G) Expression profiles (bars) and linear regression analysis (scatter) of a 377 gene G-allele discordant signature in PBMC of patients with symptomatic Huntington's disease (left set of bars), asymptomatic Huntington's disease (middle set of bars), and control subjects (right set of bars). Note distinct expression profiles of G-allele concordant signatures in PBMC from patients and control individuals.

(H) Expression profiles (bars) and linear regression analysis (scatter) of a 33 gene G-allele discordant signature in PBMC of patients with Alzheimer's disease (left set of 48 bars) and control subjects (middle set of bars). Note distinct expression profiles of G-allele concordant signatures in PBMC from patients and control individuals.

(I) Diminished clinical sample discrimination by GES associated with expression of G-allele-specific 52 nt small RNAs without segregation into concordant and discordant subsets. Designations of PBMC samples from patients and control subjects as in A-H.

FIG. 13: Microarray analysis of gene expression signatures (GES) associated with expression of rs2670660-encoded small RNAs discriminates normal and pathological tissue samples from patients with multiple common human disorders and control subjects. GES associated with expression of G-allele-specific 52 nt small RNAs in BJ1 cells was identified using t-statistics and screened for concordant and discordant features in corresponding clinical settings to segregate G-allele concordant and G-allele discordant signatures. Expression profiles of G-allele concordant and G-allele discordant signatures in individual samples of each data set were evaluated by calculating Pearson correlation coefficients (signature scores) using log 10-transformed fold expression changes of G-allele-specific GES in BJ1 cells as a multidimensional standard vector. Shaded area identifies the range defined by the average +/−2STDEV values of the signature scores in control set of samples.

(A) Expression profiles of a 102 gene G-allele concordant signature (left panel) and a 148 gene G-allele discordant signature (right panel) in normal and pathological tissue samples (brain hippocampus) of control subjects (far left sets of bars) and patients with Alzheimer's disease (right sets of bars). Tissue samples from Alzheimer's patients are segregated into three sub-sets based on clinically-defined severity of the disease (left to right): incipient, moderate, and severe. Note highly statistically significant distinct expression profiles of G-allele concordant signatures in normal and pathological tissue samples from patients and control individuals.

(B) Expression profiles of a 490 gene G-allele concordant signature (left panel) and a 299 gene G-allele discordant signature (right panel) in normal and pathological tissue samples of control subjects (far left sets of bars; normal prostate tissues) and patients with prostate cancer (right sets of bars). Tissue samples from prostate cancer patients are segregated into three sub-sets based on pathology-defined types of tissue samples (left to right): defined by histological examination morphologically normal prostate tissues adjacent to tumor; primary prostate tumors; metastatic prostate tumors in distant organs. Note highly statistically significant distinct expression profiles of G-allele concordant signatures in normal and pathological tissue samples from patients and control individuals.

(C) Expression profiles of a 29 gene G-allele concordant signature (left panel) and a 16 gene G-allele discordant signature (right panel) in normal and pathological tissue samples of control subjects (far left sets of bars; normal breast tissues) and patients with breast cancer (right sets of bars). Tissue samples from breast cancer patients are segregated into five sub-sets based on pathology-defined types of tissue samples (left to right): defined by histological examination morphologically normal breast tissues adjacent to tumor; primary breast tumors from patients without metastatic disease; primary breast tumors from patients with metastatic disease; lymph nodes from patients with metastatic disease; metastatic breast tumors in distant organs. Note highly statistically significant distinct expression profiles of G-allele concordant signatures in normal and pathological tissue samples from patients and control individuals.

FIG. 14: Microarray analysis of gene expression signatures (GES) associated with expression of rs2670660-encoded small RNAs discriminates normal and pathological tissue samples from patients with autism and control subjects (A) as well lean and obese subjects (B,C). GES associated with expression of G-allele-specific 52 nt small RNAs in BJ1 cells was identified using t-statistics and screened for concordant and discordant features in corresponding clinical settings to segregate G-allele concordant and G-allele discordant signatures. Expression profiles of G-allele concordant and G-allele discordant signatures in individual samples of each data set were evaluated by calculating Pearson correlation coefficients (signature scores) using logl O-transformed fold expression changes of G-allele-specific GES in BJ1 cells as a multidimensional standard vector. Shaded area identifies the range defined by the average +/−2STDEV values of the signature scores in control set of samples.

FIG. 15: Intergenic trans-regulatory RNAs represent a most prevalent class of transcripts containing SNP variants associated with common human disorders (A) and display cell-type specific patterns of expression in human cells (B; C).

(A) Graphical representation of the relative prevalence of distinct SNP classes defined by analysis of genomic coordinates of disease-linked SNPs identified in genome-wide association studies (GWAS) of 22 common human disorders. Distinct SNP classes were defined based on the assessment of chromosomal positions of 277 SNPs identified in genome-wide association studies (GWAS) of up to 712,263 samples comprising 221,158 disease cases, 322,862 controls and 168,233 case/control subjects of obesity GWAS.

(B) Cell type-specific expression profiles of 11 intergenic small trans RNAs containing SNP sequences associated with high risk of developing prostate cancer. Note that small transRNAs A10, A11, A18 (marked in boxes) are expressed exclusively in human cells of epithelial origin (RWPE1); transRNA A9 is expressed in cells of mesenchymal (BJ1) and lymphoid (U937) origins, but not in epithelial RWPE1 cells; transRNA A18 is expressed in epithelial RWPE1 cells and mesenchymal BJ1 cells, but not in lymphoid U937 cells; transRNA A21 is expressed in epithelial RWPE1 cells and lymphoid U937 cells, but not in mesenchymal BJ1 cells. Nearly ubiquitous patterns of expression of long noncoding RNAs containing the corresponding SNP sequences suggest a model of cell type-specific biogenesis of small tarnsRNAs based on differentiation-associated processing of long non-coding RNAs. Small transRNAs and long noncoding RNAs containing identical SNP variants are aligned in columns designated A5, A6, A9, A10, A11, A13, A14, A18, A19, A20, and A21.

(C) Cell type-specific expression profiles of six intergenic small transRNAs containing SNP sequences associated with high risk of developing breast cancer, Small transRNAs A7; A8; and B6 (shown in boxes) are expressed exclusively in human cells of epithelial origin (RWPE1); transRNA B7 is expressed in human cells of lymphoid (U937) origin, but not in epithelial (RWPE1) and mesenchymal (BJ1) cells. Note that long non-coding RNAs containing corresponding SNP sequences manifest more uniform expression profiles compared to small transRNA counterparts. Small transRNAs and long non-coding RNAs containing identical SNP variants are aligned in columns designated A7, A8, A16, B5, B6, and B7.

FIG. 16: (A) Expression of RNA A6 (SEQ ID NO:7) facilitates androgen-independent growth of the androgen-dependent human prostate cancer cell line LNCap and the highly metastatic cell line LNCapLN3. (B) Expression of RNA A6 enhances the colony-formation ability of LNCap cells in soft agar.

FIG. 17: Concordance analysis of 3299 and 1561 rs2670660 G-allele RNA-regulated transcripts.

FIG. 18: Concordance analysis of 3268 and 1636 rs2670660 G-allele RNA-regulated transcripts.

### DETAILED DESCRIPTION OF THE INVENTION

The present invention is based upon the discovery of small SNP sequence-bearing RNA molecules having gene regulatory activity. The small non-coding RNA molecules of the present invention are distinct from the non-coding RNA molecules of the prior art, which include, e.g., small and large interfering RNA molecules, hairpin RNA molecules, and microRNA molecules. See background, infra. The term “non-coding” means that the RNA molecule is not translated into an amino acid sequence. Thus, the RNA molecules of the invention do not encode proteins. The small RNA molecules of the invention are transcribed from intergenic or intronic regions of the human genome containing at least one disease-linked SNP. These small non-coding RNA molecules are referred to herein as “snpRNAs.” The snpRNA molecules of the invention are able to regulate the expression of genes distant from the genomic site of their transcription. Accordingly, they may also be referred to as “transRNA” molecules. As used herein, the terms “snpRNAs” and “transRNAs” are synonymous. The snpRNA molecules of the invention, and their corresponding DNA and cDNA molecules, are isolated and preferably purified.

The term “isolated,” in the context of a polynucleotide molecule of the invention, refers to a polynucleotide molecule that has been isolated from a cell. An isolated polynucleotide may contain various impurities which are removed by subsequent purification. Methods for purifying polynucleotides from various cellular contaminants are known in the art.

The term “purified,” in the context of a polynucleotide molecule of the invention, refers to a polynucleotide molecule that is substantially free of cellular material or contaminating proteins from the cell or tissue source from which it is isolated or recombinantly produced, or substantially free of chemical precursors or other chemical agents when chemically synthesized. Preferably, a purified polynucleotide of the invention has less than about 30%, 20%, 10%, or 5% (by dry weight) of heterologous protein, polypeptide, peptide, or antibody (also referred to as a “contaminating protein”). In a specific embodiment, the purified polynucleotide is 60%, preferably 65%, 70%, 75%, 80%, 85%, 90%, or 99% free of contaminating proteins, cellular material, chemical agents, and precursors.

The snpRNA molecules of the invention are non-coding RNA molecules transcribed from a genomic sequence containing a disease-linked SNP. Preferably, the SNP-containing genomic sequence is an intergenic sequence. An intergenic sequence is one that is distant from a protein coding region of the genome. An SNP refers to a particular kind of DNA sequence variation occurring in a population, preferably a human population, in which a single nucleotide (denoted A, T, C, or G, in accordance with the convention in the art) in the genome differs between members of a species at a particular location in the genome, also referred to as a genetic locus. The differences are referred to as alleles based on the identity of the possible single nucleotide differences. Thus, where the nucleotide at the variant position is either C or T, these variants are referred to as the C-allele and the T-allele, respectively. In a preferred embodiment, the SNP has only two alleles. Since an individual has paired sets of chromosomes, an individual is said to be homozygous or heterozygous for a particular allele depending on whether both chromosomes contain the same or different alleles, respectively. Within a population, SNPs can be assigned an allele frequency which refers to the frequency of a particular allele at a given genetic locus within the population. Preferably, allelic frequency is based upon a geographical population or an ethnic population.

By “containing at least one disease-linked SNP” it is meant that the snpRNA is transcribed from an SNP-bearing allele of a DNA molecule. In certain embodiments, the snpRNA is transcribed from one or both alleles of the DNA molecule bearing the SNP. The allele of the SNP that is associated with a disease or disorder is referred to as the “pathological allele.” The allele of the SNP that is not so associated is referred to as the “ancestral allele.”

All polynucleotide sequences described herein are written in the 5′ to 3′ orientation, unless specifically denoted otherwise.

The term “disease-linked” or “disease-associated” and synonymous terms when used in the context of an SNP refers to an SNP that has been associated with one or more diseases or disorders in a population of subjects, preferably human subjects, using methods known in the art. Such methods include, for example, genome-wide association studies of SNP variations. For example, a particular SNP may be associated with an increased incidence of the disease or disorder, meaning that individuals containing a particular allele at the site of the SNP are statistically more likely to have the disease or disorder. The statistical methods used to establish the association between SNPs and diseases or disorders are well known by those skilled in the art.

In one embodiment, the SNP is selected from the group consisting of rs2670660, rs6596075, rs6983561, rs16901979, rs13281615, rs10505477, rs10808556, rs6983267, rs7014346, rs7000448, rs1447295, rs2820037, rs889312, rs1937506, rs13387042, rs7716600, rs11249433, and rs3803662.

In one embodiment, the SNP is selected from the group consisting of, rs9469220, rs9270986, rs6457617, rs615672, rs7837688, rs6997709, rs16892766, rs2670660, and rs2542151.

As used herein, the singular form of a noun is meant to encompass both the singular and plural forms. Thus, “an isolated small non-coding RNA molecule” is meant to refer to one or more isolated small non-coding RNA molecules.

The invention provides an isolated small non-coding RNA molecule transcribed from an intergenic region of the human genome, wherein the RNA molecule is less than 1000, less than 800, less than 500, less than 400, less than 200, less than 150, less than 100, or less than 75 nucleotides and the intergenic region contains at least one SNP associated with one or more human diseases or disorders. In a particular embodiment, the intergenic region contains only one SNP. An intergenic region is a genomic region, preferably the human genome, located between clusters of genes. It is substantially devoid of protein-coding genes.

The RNA molecules of the present invention are depicted as their cDNA forms. In one embodiment, the cDNA form of the RNA molecule comprises a sequence selected from the group consisting of SEQ ID NOs: 1-101, 332, and 333. In another embodiment, the cDNA form of the RNA molecule comprises a sequence selected from the group consisting of SEQ ID NOs: 1, 4, 7, 10, 17, 22-28, 32-34, 332, and 333. In another embodiment, the cDNA form of the RNA molecule comprises a sequence selected from the group consisting of SEQ ID NOs: 1, 7, 332, and 333.

The invention also provides a vector comprising a polynucleotide molecule of the invention. In one embodiment, the vector comprises the cDNA form of an RNA molecule described herein. As used herein, the term “vector” in this context refers to a cloning vector or an expression vector, or both (i.e., the same vector may be designed for cloning and expression). The terms are used consistent with their common meaning in the art. Thus, a cloning vector refers to a DNA molecule, typically a plasmid molecule, into which a foreign DNA fragment can be inserted, e.g., by restriction digest and ligation. Non-limiting examples of cloning vectors include genetically engineered plasmids and bacteriophages (such as phage X) or other viruses, as well as bacterial artificial chromosomes (BACs) and yeast artificial chromosomes (YACs). An expression vector is typically engineered to contain regulatory sequences that act as enhancer and promoter regions and lead to efficient transcription of the foreign DNA. In a preferred embodiment, the vector is a viral vector. In one embodiment, the vector is an expression vector. In another embodiment, the vector is a cloning vector.

The invention further provides a cell comprising said vector. Preferably, the cell is a mammalian cell and most preferably a human cell. In a preferred embodiment, the cell stably expresses the vector.

The invention also provides a kit comprising, in one or more containers, a vector comprising a polynucleotide molecule of the invention. In one embodiment, the kit comprises an RNA molecule described herein and instructions for expressing the RNA molecule from the vector. In one embodiment, the kit comprises the cDNA form of an RNA molecule described herein and instructions for expressing the RNA molecule from the vector.

In one embodiment, the kit further comprises one or more polynucleotide primers for amplifying the cDNA molecule. In one embodiment, the one or more primers comprises a sequence selected from the group consisting of SEQ ID NOs: 102-331. In one embodiment, the one or more primers comprises a sequence selected from the group consisting of SEQ ID NOs: 102-161. In one embodiment, the one or more primers comprises a sequence selected from the group consisting of SEQ ID NOs: 102, 103, 114, 115, 326, and 327.

The invention also provides a kit comprising, in one or more containers, a cell comprising said vector and instructions for expressing the RNA molecule in the cell.

The invention also provides a method for detecting the small non-coding RNA molecules described herein in a sample from a subject, the method comprising detecting the RNA molecules in the sample. In one embodiment, the step of detecting the RNA molecules comprises the step of detecting the cDNA form of the RNA molecule in the sample. In one embodiment, the cDNA form is detected by a method comprising reverse transcription and polymerase chain reaction (RT-PCR) technology. In one embodiment, the method comprises the technique of nested PCR. These terms are used here in accordance with their normal and customary meaning in the art. Thus, “RT-PCR” refers to a PCR technique in which reverse transcriptase is first used to reverse transcribe RNA into its complementary DNA, also referred to as cDNA. The cDNA is then amplified by PCR. PCR is a well known technique used to amplify a particular DNA molecule of interest, typically from a mixture containing a high background of non-specific DNA molecules. Nested PCR employs two sets of primers in two successive PCR reactions to achieve increased specificity.

In one embodiment, the method further comprises the steps of isolating the small RNA fraction from the sample and converting the RNA into cDNA prior to the step of detecting the cDNA in the sample.

In another embodiment, the cDNA form of the RNA molecules is detected by a method comprising nucleic acid hybridization technology.

The invention also provides a method for evaluating the risk that a human subject will develop a disease or condition associated with a specific allele of an SNP (“the pathological allele”) by detecting the presence of an RNA molecule of claim 1 in a sample from the subject, wherein the RNA molecule is transcribed from the pathological allele, and wherein detection of said RNA molecule indicates that the subject has an increased risk for developing the disease or condition and the failure to detect said RNA molecule indicates that the subject has a decreased risk for developing the disease or condition.

In one embodiment, the method further comprises detecting the expression level of the RNA molecule transcribed from the pathological allele relative to its expression in a population of healthy subjects, wherein an increased or decreased level of expression relative to the population of healthy subjects indicates that the subject has an increased risk for developing the disease or condition.

In one embodiment, the step of detecting the presence of an RNA molecule transcribed from the pathological allele is performed indirectly, by detecting the expression of one or more genes whose expression is regulated by the RNA molecule.

The invention also provides a method for diagnosing a disease or condition associated with a specific allele of an SNP (“the pathological allele”) in a human subject, the method comprising detecting the presence of an RNA molecule of claim 1 in a sample from the subject, wherein the RNA molecule is transcribed from the pathological allele, and wherein the disease or condition is positively diagnosed if the RNA molecule is detected in the sample.

The invention also provides a method for treating, preventing, or ameliorating a disease or condition associated with a specific allele of an SNP (“the pathological allele”) in a subject in need thereof, the method comprising administering one or more therapeutic agents that act to suppress the expression or antagonize the activity of an RNA molecule of claim 1, wherein the RNA molecule is transcribed from the pathological allele.

As used herein, the term “subject” refers to an animal, preferably a mammal including a non-primate (e.g., a cow, pig, horse, cat, dog, rat, and mouse) and a primate (e.g., a chimpanzee, a monkey such as a cynomolgous monkey and a human), and more preferably a human.

Preferably, with respect to any of the methods described above, the subject is human.

In certain embodiments of the methods described above, the sample is a blood, tissue, or cell sample.

In a specific embodiment, the disease or condition is selected from the group consisting of Crohn's disease, rheumatoid arthritis, Huntington's disease, Alzheimer's disease, breast cancer, prostate cancer, autism, and obesity.

The invention also provides an apparatus for evaluating a disease or condition, or evaluating the risk of developing a disease or condition, in a subject, the apparatus comprising a model configured to evaluate a dataset for the subject to thereby evaluate the risk of disease in the subject, wherein the model is based upon determining the similarity in the expression profile of a defined set of genes in a sample from the subject and the expression profile for that set of genes in one or more reference sets of the model, wherein a reference set comprises one or more of a population of healthy subjects and a population of subjects suffering from the disease, wherein the set of genes is a set of genes whose expression is regulated by a small RNA molecule of claim 1.

In one embodiment, the disease or disorder is selected from Crohn's disease, rheumatoid arthritis, bipolar disorder, Alzheimer's disease, vitiligo, ulcerative colitis, type 1 diabetes, type 2 diabetes, autoimmune thyroid disease, coronary artery diseases, hypertension, multiple sclerosis, obesity, and epithelial cancers. In a specific embodiment, the epithelial malignancy is selected from prostate, breast, ovarian, and colorectal cancer.

snpRNA Molecules and Primers for their Detection

The snpRNA molecules of the invention are a novel class of non-coding RNA molecule transcribed from intergenic SNP-containing regions of the human genome. This class of RNA molecule is defined by the following structural features. The RNA molecules of the invention each contain a disease-associated SNP. The disease-associated SNP is located within a loop structure of the RNA molecule. Preferably, this loop structure containing the SNP also contains a binding site for an miRNA molecule. Preferably, the SNP is located within a binding site for one or more of the following proteins: H3K27Me3, CBP/CREB, Ezh2, and POL2. In certain embodiments where the SNP is located within the binding site for more than one protein, the binding sites overlap. In another embodiment, the SNP is within the binding site for a nuclear lamina protein. In a specific embodiment, the SNP is located within 200 basepairs of a binding site for a lamin B1 protein.

The invention provides isolated snpRNA molecules, their cDNA counterparts, and primers for their detection in a biological sample using, e.g., reverse-transcription polymerase chain reaction (RT-PCR) technology. In certain embodiments the isolated snpRNA molecules are purified. In some embodiments, the snpRNA molecules are in the form of their cDNA counterparts. The snpRNA molecules of the invention are polynucleotide sequences comprising the bases adenine (A), guanine (G), cytosine (C), and uracil (U). The counterpart cDNA molecules are polynucleotide sequences comprising the bases adenine (A), guanine (G), cytosine (C), and thymine (T). The sequences are denoted as strings of these bases, in accordance with the common practice in the art. The sequences of the present invention are denoted as cDNA sequences of the corresponding RNA molecules. The corresponding RNA molecule is easily envisioned from the cDNA sequences depicted here using methods routine in the art.

In one embodiment, the snpRNA is an allelic variant. An “allelic variant” of an snpRNA molecule of the invention refers to the allele of the SNP from which the snpRNA is transcribed. In one embodiment, the snpRNA corresponds to the pathological allele of the SNP. In another embodiment, the snpRNA corresponds to the ancestral allele. In particular embodiments, the snpRNA is an A-allele RNA, a G-allele RNA, a C-allele RNA, or a T-allele RNA, wherein the reference to the particular allele is in the context of the SNP which encodes the RNA.

In some embodiments, the snpRNA molecule of the invention is an SNP-containing fragment of a larger RNA molecule. In one embodiment, an snpRNA molecule of the invention is a processing variant of a longer non-coding RNA molecule.

Preferably, the snpRNA molecules of the invention are molecules of 50 to 300 nucleotides in length, each containing at least one disease-linked SNP. In specific embodiments, an snpRNA molecule of the invention is about 25, 50, 60, 70, 80, 90, 100, 125, 150, 175, 200, 225, 250, 275, or 300 nucleotides in length. Preferably, the snpRNA molecule is between 50-100, 50-75, or 50-60 nucleotides in length. In specific embodiments, the snpRNA molecule is about 50 nucleotides in length. In certain embodiments, the snpRNA molecules comprise about 50, 60, 70, 80, 90, 100, 125 or 150 nucleotides flanking a disease-associated SNP. Preferably, an snpRNA molecule of the invention comprises 50, 60, 70, 80 or 90 nucleotides flanking the SNP.

In one embodiment, the snpRNA molecule is contiguous. As used herein, the term “contiguous” in the context of an snpRNA molecule means that the snpRNA molecule is a single sequence, uninterrupted by any intervening sequence or sequences.

In one embodiment, the snpRNA molecule of the invention acts as a transcriptional suppressor on one or more genes encoding proteins selected from the Polycomb group (PcG), the bivalent chromatin domain (BCD) group, NALP1, NALP3, and the PluriNet group. The term “Polycomb group” refers to a family of chromatin remodeling proteins that function in the epigenetic silencing of genes. The terms “NALP1 and NALP3” refer to proteins that assemble into complexes called “inflammasomes” which activate caspase-1, resulting in the processing of pro-inflammatory cytokines and triggering an innate immune response. The term “PluriNet” refers to a protein network common to pluripotent cells which enables them to differentiate into multiple cell types. See e.g., Müller, F. J. et al., Regulatory networks define phenotypic classes of human stem cell lines, Nature 455:401-405 (18 Sep. 2008).

The invention provides isolated snpRNA molecules and the cDNA counterparts of the RNA molecules. The following tables give the cDNA sequences of the snpRNA molecules of the invention. Each sequence in the table below represents two sequences, one for each allelic variant of the SNP. The two sequences for each allelic variant are identical except for a single nucleotide at the position indicated in the sequence as variable. The variable position is denoted in the sequence as, e.g., “[G/A]” which indicates that one allele contains a “G” at that position in the sequence and the other allele contains an “A” at that position in the sequence. The sequences below are referred to as “cDNA” sequences because they are the DNA sequence complementary to the RNA molecules transcribed from the genomic DNA.

The intergenic RNA molecules of the invention are represented by their respective cDNA sequences in Table 1. Additional RNA molecules identified or predicted to be encoded by intronic sequences are represented by their respective cDNA sequences in Table 2. Primers which can be used to amplify the RNA molecules of the invention using reverse transcription followed by a polymerase chain reaction are shown in Table 3.

TABLE 1
cDNA sequences of small snpRNA molecules transcribed from intergenic SNP's.

| | | SEQ |
|---|---|---|
| | | ID |
| Name/SNP | SEQUENCE | NO: |
| rs2670660 | CACAAGTGATCTACCAGTCTTTTAAA[G/A]TTCTATTATTAAAACCCAAACATGC | 1 |
| A1: rs6458307 | TCTTTAATACAGATTGGGAAGAGGATTACTTTTTCTGTCTCAGGTTCTTCAGGATAAAGGAT | 2 |
| | AAAGATTTGGAGATCGTTTAAAAGCTTTTATATAAATGCTCATTCA[C/T]TGAGTTCAAAT | |
| | ACTTTTAAAATGTCCTGGCAGTTGAAAGTTA | |
| A2: rs9472138 | GAACACTTCTGTTACCCTAAGCACGTTCTCCTCATA[C/T]CGTTTGTCGTCAATCCCTACC | 3 |
| | ACGGCTACCAGTCTCAGGCAGCTACTAATCTATCTGCTTTTTTTCTGTGTAATTTTGCCTTT | |
| | TCCAGAAAGTC | |
| A3: rs6596075 | ATTTGTGTTCAAGCCTCCTTCCATGGGAAGAACCAGCGGTGGACCTGAAGAGCTCTGCCTTC | 4 |
| | AAACAGATGATTCACTCA[C/G]AACAGGTTGCTGGTGACTGAACCTCAGTGA | |
| A4: rs2544677 | TAATCTTTGTCTTTATGAA[C/G]GTCTAGAGGATTCTACCATAAAATTAGGAAAGATAAGT | 5 |
| | TAGAAATGTTGAAACATAGAAAGTATTATAACTAGAACGCATTTAATACTTGTATTTTTAAT | |
| | TTTTGAGACAGTCTTCCTCTGTCACCCAGG | |
| A5: rs6983561 | ATAGAACATATAGCAC[A/C]AAATGATTATATCAATAGAATGCTAATTGCATATCAAGGAT | 6 |
| | ATTTGGTATAATACAAATTATTCTACCTTAAACATATGGAAATTTGTGGTCCATGA | |
| A6: rs16901979 | AGTGTGGGGTCTTTGTTGTGGAGCAGTGTTAATGATTTAGCATTACTTAT[A/C]TCTGGCA | 7 |
| | AATGGTATTTTTGAGATAACATGTTATGGAAGAAAGTGAACTGAACTTGGAAGTTTGAAGAT | |
| | CTCGATTGAAGTATC | |
| A7: rs672888 | GGGCATTTTCTGTGCTACTATTCTTAAGAGAATTATCTCACTCAATCCTCACTGCAGCTCTA | 8 |
| | GGAGCTAGATACTGTTATTG[C/T]CACTTTCTTA | |
| | AAGGTAAAGAAACACAGATATTAGGCCTATTGCCAGCATCACTCAGCA | |
| A8: rs13281615 | GACACGTGGAATTTACTCTTTTGATAAATTGGTAACTATGAATCTCATCAAAAGAA[A/G]G | 9 |
| | CAGAACGCAGATATTCTGAGTAGGGGGTTTGGGGGAGAAATAAGAGTGATTCCTCCTATCTG | |
| | CTGCTAGGGCCATAAAGACACTACACCAAGAGGAAGTGTAGGCTTGGCCAGGT | |
| A9: rs10505477 | CCGTGGGAAACAAAGTCTTCCACTGGGCTTATTCTGTGTCATGTGTC | 10 |
| | ACCACTTGTCTATCAAACAGGAAGCCTTAA[C/T]TGGAGATGAA | |
| | GATTTAGAAAAGGGGCAAAGTCAGTATTGA | |
| A10: rs10808556 | CTCCATAGAGCCTGCAGAGGGCACTAGACTGGGAATTAGAAAACCTGATTTCCCTTCCAGCT | 11 |
| | CCA[C/T]CTCTGACCAATTGCCTGACCCTGGTCAAATTGCTTAACCTCTTCCTATCTCAGC | |
| | TCCCTATCCATAAAACAGAGGGACGAATAAA | |
| A11: rs6983267 | TCCTTTGAGCTCAGCAGATGAAAG[G/T]CACTGAGAAAAGTACAAAGAATTTTTATGTGCT | 12 |
| | ATTGACTTTATTTTATTTTATGTGGGGGAGGGAGCCGGCCCCAGCTGGAAAGCTGCTTTCTC | |
| | TGAATCAAAGGGCAGGAACCCAGCAAGTTTCTCA | |
| A12: rs7014346 | GCTTGCAGCTTCTGCCTAATGTTGACTTACAGTTCAAGATGGCTTCTGGAGTGCTACC[A/G] | 13 |
| | TTACATCCATGTTGTAGGCTAGAAGGAAAA | |
| | GGGCAATGGCCTGAAGAGGAAGGGAGAGTTCCTGTTA | |
| A13: rs7000448 | GAGCAGAGGAGCAG[C/T]ATTTTTGAGAATCTGGCCAATATGGAAAGATTTGCTGACATAT | 14 |
| | TCAGATTTGAGACTTTTTTTTTTTTTAGACGGAGTTTTGCTCTTGCCACTCAGGCTGGAGTG | |
| | CAGTGGCACAATCTC | |
| A14: rs1447295 | TGAGTTGCACGCCAGACACTATACTAGATGATGGGACAACTAAAGGGTAATGAACAGTTCTG | 15 |
| | TCTCTATGTAAAAATAATAATGATGATGATGATGAGATGGGACTTCAATTGAGGAAGTGCCA | |
| | TTGGGGAGGTATGTAAAA[A/C]GTGCTATGGAAAAAAAGCAACAGGAACCCCT | |
| A15: rs2820037 | GTGATTGCTCTAATTGCCAAGTACAGAAAAAGTTACTGGGTGTGTTCATAGATCTAGTAGCT | 16 |
| | CTATTGTGAGGTGAATTTTAGTCAGGACTTCAATTATCACATAGTTTTCTTGAGCCTCCA[A/ | |
| | T]TCTAAAAGAGAGCCTGTGATTACTCTTTTGTTCTTTAGGTATTAACATCAACATAGACC | |
| | TCATGCGC | |
| A16: rs889312 | ATGCCCCTGCTGGAGAAAGG[A/C]ATGTGCAAATTAAGAGACTA | 17 |
| | CAAATCAGTTTGAAAACTCAACGACTCCTTCCCA | |
| A17: rs1937506 | CGGGAAAGTAAAAATTGTTATCTCATTCATATTCAAAAATTTGATAAAA | 18 |
| | TCAGGCTTGGAAAATGTGATTTATTAGGTGTCAAATAATGAAGTTATACCTGTGGAGA[A/G] | |
| | TATTAGAAGTGGAACATTGTAATGGATATGTCCAAAGGATTGGTCCTC | |
| A18: rs4242382 | CCCAGGGAACATTTTGTCCCTCTAGTTATCTTCCC[A/G]CAGGCCCATCAAGAATCAGGCA | 19 |
| | GTAGGTGAAAAAGAAACACAGAGAACCTAGGAACACAATAG | |
| A19: rs7017300 | GAGCCAGGACATCAGAAAGAAAATTAAAAACAAAGTGGAATACAGTGTGAAGATTGATTTGG | 20 |
| | GGCAAAAGATTTGAAACTAAGACCATGAACAAT | |
| | GAGATTCGTTAATGGAGTTTCCCTTTGTATGATGCCTAGA[A/C]CCAGCAACAGGGCAGTT | |
| | GCAGTGATTTAAGGATGACTCACAGGGATGG | |
| A20: rs10090154 | TTCTCTCCAGATTGATACACAGCTTTAATGCAATTCTTATAAAAATCTCTGCAAGATTTTTT | 21 |
| | TGTAAA[C/T]ATAGCTAAAACAATATTGGAAAAAAAATAGTGAAGTGGTATTCCAAGGCTT | |
| | ACTATATGGCCAGAGTAGTCCAGACTGTGGTATTGGCAGAGGCATT | |
| A21: rs7837688 | TTCACAGGAAAATTGAGCAGAAAGTACAAAGAGCTCCTGTATATCCCCTACCCCCACACATT | 22 |
| | CACAGCCTCCCTCATTACCAACATTTCCCACTAGAGTGGTGCATTT[G/T]GTACAATTGGG | |
| | TCTATGTTGACACGTCATT | |
| A22_1: | TGCTCCTGTCTCCCAAACTCTAGATGCCACGTGGGCGCTGTAGCCCCACTTCGCCAATGCCT | 23 |
| rs2542151_1 | TGGTTCGGGC | |
| A22_2: | GGGC[G/T]CTTCCTGAGACTCTCATTTTCCTAATTTCACTAACTTCACACCTTCTTGCTAA | 24 |
| rs2542151_2 | TTCTGATTATTTTTCCTCTGCGATAGGGA | |
| A23: rs16892766 | ACGGTCAGACGCAAACAGTTTCAAGACTATT[A/C]GCTGTTAAAG | 25 |
| | GTTATGCCTTATGTCACCCAAAAGGGTTTTCCCCTAGATTTATAGCACAAACTCATGGAAGA | |
| | TTTATTGCCGTCTTAATTTTTTCCCCAATTTTAACTTTA-A/C]GAACAGTCAGCCTG | |
| A24: rs6997709 | TTGACCAAATTGAAGAATTGGTTTGTTCTCACCTAAGTTCTATCAAGCCAAATAAGT[G/T] | 26 |
| | ATGGGACAGGATGAAAAAGATTTTTCCTGACGTGAAAGGATTTGGGTAGTCACCCATTGAAT | |
| | GTTCTCATGGAGATCAAGTCT | |
| A25: rs6457617 | TAGTCA[C/T]ATCTGCTCATGGACTCAACAAACAGTAATTGAGTCCACTGACTGCATTTCG | 27 |
| | GAAATCCACACTCATGATCTTCCTCTG | |
| A26_1: rs9469220 | ATAAATTACCATTCAAACTGCC[A/G]GTAGAAATATAAAATTGTAAGGAATAAATTCCACA | 28 |
| | AAAAAATACAGTGTTTTAATTACAAAAATTTACCATGCAGCA | |
| A26_2: | TGGCAGTCCAAGCTACTAAGAAGCACAAATAAAATATATAGTAGCAGGGGGAGATGGGAAGG | 29 |
| rs9469220_2 | GTGAGAGAATGTAGGATAAATTACCATTCAAACTGCC[A/G]GTAGAAATATA | |
| A26_3: | TGGCAGTCCAAGCTACTAAGAAGCACAAATAAAATATATAGTAGCAGGGGGAGATGGGAAGG | 30 |
| rs9469220_3 | GTGAGAGAATGTAGGATAAATTACCATTCAAACTGCC[A/G]GTAGAAATATAAAATTGTAA | |
| | GGAATAAATTCCACAAAAAAATACAGTGTTTTAATTACAAAAATTTACCATGCAGCA | |
| A27: rs660895 | CTGTCTGATGGGAGTGAAGATTCTTCCTTCAGGAATGGAAGGGGATGCACAGAGTGAAGCCA | 31 |
| | CCCAACAAAAACAAGACTTGTAT[A/G]GCTATAGATGGAAGGGAAATCAACCAGGAAATTA | |
| | TTTTGG | |
| A28: rs615672 | GTGGTTAGGAAAA[C/G]AGAAATAAGAACAACAGCAGAATGCACCGT | 32 |
| | CAGGTACTTTGGAAGTCACAGAAGGGAAAAGGGCAGG | |
| A29_1: | ATGTTCATCAGTGGTCACAAATATAATGTATCTAAAATAGGGACAGTAAGAAATTACTGGGC | 33 |
| rs9270986_1 | ATAACTAG[A/C]AGGTGCCATGGGATGTGCCTGGAAAGCTTCTCATGACGACCTACCATGA | |
| | GCC | |
| A29_2: | ATGTTCATCAGTGGTCACAAATATAATGTATCTAAAATAGGGACAGTAAGAAATTACTGGGC | 34 |
| rs9270986_2 | ATAACTAG[A/C] | |
| B1: rs10186922 | CAGCTCTGACTCCCAACTCCACACCCCCATGTACTTCTTCCTCTCCAACCTGTGCTGGGCTG | 35 |
| | ACATCGGTTTCACCTCGCCCATGGTTCCCAAGATGATC[A/G]TGGACATGCAGTCGCATAG | |
| | CAGAGTCATCTCTCATGCGGGCTGCCTGACACGGATGTCTTTCTTGGTCCTTTTTGCATGTA | |
| | TAGAAGACATGCTCCTGACTG | |
| B2: rs11159647 | TGCTCACTACCTGGGTGCAATATACTCATATAGCAAAGCTGCACAT[A/G]TATCTAACATA | 36 |
| | ACATTGAAATTTTAAAAATAGGACATTTTAATACAAAATTAGATTTAAAAGTAATTACTATT | |
| | AGCGAAAATAAGTCACAACCATTTAGAAATCTGAAAAATGCTGACAA | |
| B3: rs2609653 | TGTGCACAAGAGCATTGTTTTCTAGCATATACTTATTTTAACTATTTTTAGAAGCA[C/T]T | 37 |
| | TCGCATTTTGAAAAGTGAAAATAACCTAAGTGTTCATCAATGGATGAATGGAAAAAGAAACT | |
| | GTGGTACGTATATACAATGGAATATTATACGGCTCTAAAAAAGAATGGGATCCTGCCATCTG | |
| | TCACAACATGGATGATCCT | |
| B4: rs7570682 | AGTGATGGAGTGGCATAGGTAATTTCTGGAATGACTGAAGTAAATATAATCAGCTCACTTTA | 38 |
| | AAATGAATTTTTTCAGTATAAAGTAACTCTCTGGAA[A/G]TTGACATGAAGTTTGATCAGA | |
| | AATTAAGGCAGAAGGTATGTGAAACAGTAGAAACTGTAGATATGAGTATAAAAAAAGTGGGT | |
| | GGCAAGGGATAAGGAAGCATGTAGGG | |
| B5: rs13387042 | CAGAAAGAAGGCAAATGGA[A/G]GCTACAGAAACCAAGGATTTCCTTGTTGAATCGAATCT | 39 |
| | TCCTTCAATCTTCCTTCACCACACTAGTGGATCTCCCTGTGGGAGGGATGTTGAGAGTGCTC | |
| | CGTGTTTTTT | |
| B6: rs2291533 | TTTTTTAATTTATACTTCCTCATGGTTCTCTTGGATATCCTCTGGAACTGTTTAGAAGACTG | 40 |
| | AAGAATTTCATCCCCCAGAAACTCACA[C/G]TGTTGAAGCTCAGCATGTCTTTGGGCCAGT | |
| | AGCTT | |
| B7: rs2822558 | TTCTCGACAAAAGTTTTCCACTGGGGAAATTATTAACTTGATGTCAGCAACTCATGGACTTG | 41 |
| | ACA[A/G]CAAACCTCAATCTCCTCTGGTCTGCCCCTTTTCAAATCCTAATGGCCGTATATC | |
| | TCCTTTGGCAAGAGCTGGGTCCAGCAGTGTTAGCAGGG | |
| B8: rs10795668 | TTGTTTTCAGGAGTTTTCATCTATGAGCAGCAGCAGAAAGAGAAAAAGTTAGATTCTTA[A/ | 42 |
| | G]ATTCCATGATTTTATATTTCCCACCAAGGTACAAGTATTTCTACTTTTCTACCTGATTGT | |
| | CTCTACTTTCCTCCATGTGTATTTCTTTTCTTTTCTTTTCTTTTTCAGACGGAGTCTCGCT | |
| B9: rs4779584 | AGCTGCTATAAGATGGGCTGAGTTAGAAAAACCTAACAGCCCATCCTAATAGACTGAATGTT | 43 |
| | CTATTGTTTGATGAATGTTATGTGCCAGTAGAACTTGTTGATAAGCCATTCTTC[C/T]GAA | |
| | CAGAAACCATAACTATAYACACAGGAAACAAAAATATTTGTAATGGCTTTTAGCAGTGGCAA | |
| B10: rs10757274 | AGCTTCTCCCCCGTGGGTCAAATCTAAGCTGAGTGTTG[A/G]GACATAATTGAAATTCACT | 44 |
| | AGATAGATAGGAGATAGGGGTAGGGAATTCTAATCAGAGGGAATAGCACATGTAAGGCAAAC | |
| | AATACAGTGCATCTGGGAAAGCTATACAATTTTATTGTTATAGGACAAATGTTGGGGAATGT | |
| | TGAGAGATGGAACTGGAGAGTGAGGCAG | |
| B11: rs10757278 | GTTAAGTTAGTTGGAACTGAACTGAGGCCAGACAGGGCTGTGGGACAAGTCAGGGTGTGGTC | 45 |
| | ATTCCGGTA[A/G]GCAGCGATGCAGAATCAAGACAGAGTAGTTTCTCCTTCTCTCTCTCTC | |
| | TTTAATTGTAACG | |
| B12: rs1333049 | TCTGCTTCATATTCCAACTTGTGTATGACACTTCTTAGGCTATCATTTCATTCCAAATTTAT | 46 |
| | GGTCACTACCCTACTGTCATTCCTCATACTAACCATATGATCAACAGTT[C/G]AAAAGCAG | |
| | CCACTCGCAGAGGTAAGCAAGATATATGGTAAATACTGTGTTGACAAAAGTATGCAGAAGCA | |
| B13: rs2383206 | TGGCCCGATGATTTTCAGTTAACCAAATTCTCCCTTACTATCCTGGTTGCCCCTTCTGTCTT | 47 |
| | TTCCTTAGAAATGTTATTGTAGT[A/G]TTTGCAAGATGGCCTGAATCCTGAACCCCCCATC | |
| | TTCAATGAGCACCAAATGGTAATTATAGATTCCCAGCTGTAGAGCTATGTCAG | |
| B14: rs2383207 | ATACTTAGCCCTTGGGACCATTTTTTACTCCTGTTCGGATCCCTTC[A/G]GCTAAGCATGA | 48 |
| | TTATTTACTATTTTCAGCTATTAGTTATGTCTTGTTGAAAAAGTATGAAAAGAGCTGCCCAA | |
| | TAAATTAGAGTGTATGCTCAACATTCTCTTAGCTTCTT | |
| B15: rs383830 | CCTGATGTAAACTACTCTTTGTTCAACCCTTAGTAGTACAAATATGATACTTTATTTTTACT | 49 |
| | GTTACTCATGTTGCCTTGAAAACTCCTGTGTTCTGTTATCTTTGAATGTGAGCTAGT[A/T] | |
| | ACTTTATTTTAATTTTTGGAAGTCCTGTGGGTGTAAATTG | |
| C1: rs7250581 | CTCCAAAAGCCAGGAGAATGGGAGGGAAGTGAGGGTTGAAAAATTACCTATCAGGTAGAGTG | 50 |
| | TTCACTGTTCGGGAGGTGGGTTTGCTAGAAGCTCAATCCCAACCATTAC[A/G]CTATATGC | |
| | CTATGTAACAAACACACACATATACTTAAAATTTGTTTTAAAAACCCAAATTTCTGGCTTCT | |
| | CCTGAAAAAAATATAATATGCAGCCACACGGG | |
| C2: rs10733113 | CACAGTCTGTTACAAGGGTGGAATGAATTGTTTCTTGTAAAGCACTCAGAACAATGAGTGGC | 51 |
| | ACAGAGTGATACATGTTGAGGGCTTTTTGTTGTTGTTGTTGTTGAT[A/G]TATTGTCTCAG | |
| | CACCCTATTATATTTTTCACATGGAGGGGATAAAAAAAATCTTTCTTAAGACAGGCCGCAAG | |
| | AAGTA | |
| C3: rs10761659 | ACTGAAAGTGCTCCTTCACAAATGAACACTTAAATTCAGGAGCACTTTCAGTTAAAGCAAAG | 52 |
| | GAGTTAAAGCAAAGACTTTGGGAGTCAGTATCAAATAAAGATCATCTCTCAAACT[A/G]TA | |
| | ACAGAAGGAAAACAGGAATTAATTTATTTCAGACTTTTTAGAAACGCCCTCCTCTTTGACT | |
| C4: rs10883365 | GCCGCATAAGACGTTACTTAAACATGTTACTTAAACAAGACTGCAGTAAACGTTTCTTTCCA | 53 |
| | AGTGAGAAAGGTCTTTTTCGTTCTCAGACGGTTTGAAGGT[A/G]TTTGTGCCAACGTGACC | |
| | CCCGGGGAGATTTGGAGGAAGCTTTCTACGTCCTAGGAGGCTGAGATCCCACGGAGCCGGTT | |
| | TACGGTTGAGAGCAGACAGTTTCGAGTAGATAGCGCTGGAAGAGACACGAA | |
| C5: rs17234657 | AGTGCTGAAGCGGAATTGAGCTCCTTAAGTTTTGTACATCATGTTTTTTTAGGTTCCCACTG | 54 |
| | AGCTGATTTTTGGCCATGATTCACACATATCTCTCCTCCAAGGCTCCTCTCACAAAGCATTT | |
| | CCTCCCAGTCACGTT[G/T]TCAAATAGCTTCTCATTCCCTGTATGCCTGTGTGTGCATGGC | |
| | CTCATCTCACTTTCGCTGTGACCATTGCTGCTCAT | |
| C6: rs55646866 | AGAGTCCTCAGCCTCGTCAGTTATTCCTTCTAGTGCTGGGGACGAAGGGAAGAGGAGGAGAA | 55 |
| | GGAGCTGGGACCCAGCAGTGATGGGCCTATGGGAGGGAGGATA[C/T]GGCTGCACAGCCCT | |
| | CAGCGCGTGGCTCAGGCAGGGTCAGCCCCTCTGCACATGCCTCCCCCTACCACCACCACACG | |
| | TCATCGCCTTTTTATGTGGTCTGACTTTTTCAGATTTTTCAACCTGAAGCTTGCTTTCTC | |
| C7: rs6672995 | AGGGTTCCTGGCTCCTACAGAAGACTTGCTTTAGGACTGAAGGCTATATTGCAGTCTGTGTT | 56 |
| | GGCCTTAGTCGCGGAGGGACATTTAA[A/G]GATGGACTTACTAGAAATGCTCTTCATATTC | |
| | CAGGAACACACAGCACATTTCCTCTGATGGGCTGCTGGGACCTTACCATTTACTGGAACCCA | |
| | ACCCTCTGA | |
| C8: ss107635144 | ACTAGAGTGTGTGATTCAGGTAAAGCATGAGACCTGAACTGGCTTCAACACCAGGCT[C/T] | 57 |
| | GGTCACTCATGCCATGTGTCTTTGAGCAGGTTACTTAACCTATCTGTGCCTCACTTGTGTTT | |
| | TCTT | |
| C9: rs12037606 | TCTTAGTACATACGTTCCAAAT[A/G]TGAATCAGCTGTGATAAAGCTTGTCAAAACACTAA | 58 |
| | CTTAGTCTTAGACTGGGAACAGTACTAAAATAAAGGGAATGTTAGATGTTGCATACCATGAA | |
| | CAGCTGAGCTACCT | |
| C10: rs6601764 | ATGGTTTTGAGCTTTCAGAGGTGACAGGAGT[C/T]AAGTAAGTGAGTTTATGATGTAAGCA | 59 |
| | CACTTGAATGCTCCTTTAATCTTTAGAGCGGGGGCCACTGATCTTTGTTAATTTCCACAAAA | |
| | TCTCTGCAAAGCCGCGTTCTTCCTGGATTACTCAGAAAAGCCTTCCAGATGGTGA | |
| C11: rs7807268 | CTCTCTCTCTAAATGCCTTGGGACCATCATGTCTAACCCTTCGCTACAGACATTGGTGAG[C/ | 60 |
| | G]ACAGCTTAGGCCATGGTGATGTTCATACTGTAGTGTCCAAACAGGAGGAAATCACCCTT | |
| | CCAGTCCCTT | |
| C12: rs6957669 | TGGTGGTGATTACTGCCCTTGCTGGGGGTCACACAGATGCATCTGGGAGGATCTGGAAGGGG | 61 |
| | CCTGCCCCTCTTGAGCTTGGAGCTCCCTCATATG[A/G]GTTCACCAGTGAGGACACAGTCA | |
| | TTGTTGGTTAGAGACTGGGACTCAAGTTGTAGGCTCCTTTCAGTCTTTGCGTCA | |
| C13: rs12970134 | ACTGACTCTTACCAAACAAAGCATGA[A/G]CAAACAAAGATTTATCAGAAGGGTG | 62 |
| C14: rs17782313 | CTTGGAAGCAGGAAAACCAGAATATATGTGAGCATCTTTAATGACTACAACATTATAGAAGT | 63 |
| | TTAAAGCAGGAGAGATTGTATCC[C/T]GATGGAAATGACAAGAAAAGCTTCAGGGGGAAGG | |
| | TGACATTTAAGTTGGAATATTATTGAGGAGTATCATTTTAGCATCTGGGATTGAGGTAGC | |
| C15: rs1859962 | TCACAAAGAACACCTTGGACCAGTTCTTGATATAAATAAGAGGCTGCAGACTTTTCCAAATC | 64 |
| | CCTGCCCGTG[G/T]GATGAACACTTTAAAGGTCCCAAGATTTCTAATAATGGGGCTAAATT | |
| | TCCCAAAATGTG | |
| C16: rs983085 | GGAATTGTACACCATCACCAAATATGGCATATACCAGGTATGTGAGGCTGGTTCAATATTTG | 65 |
| | AAAACCAGTCACTGTAATACACCCT[A/G]TTAACAAACTAAGGATGAAAAATGTACATGAT | |
| | CATAACAATCAATGGAGAAAAAGCATTTGACAAAA | |
| D1: rs10490072 | TTTGAAATGCAAGCTCCAAGAGAGTGAAGCCCCAGCCTGCACTGCCTTACTTTGTGCAGAGA | 66 |
| | ATGCTTCTTTGGTTATGTATATACATGC[C/T]TGCTTATTCTAATCCATGCCTTTATTACG | |
| | AAATTCATCTAATGTTGTGGCCAAATGGCAATAAAATAATATTATTACAGGACACGGGCCT | |
| D2: rs1153188 | GAAGATGGTCTGAATGGCAAAATGGATAAAATTAAAATCAAAACTAGTGAACTGAAATAGCA | 67 |
| | AGGTGAGAAGTTCTTCTGAA[A/T]TGCAGTATAAAAGATAAAAAGAAATACAAAGAAAAAG | |
| | TCATGAAGGACAGATCCAGTGGACGAAACA | |
| D3: rs13071168 | CCCACATCCAGACTTCTGCTCTGATTCTCACTTCCACTCACCACACGTACCCATCTGTTCAC | 68 |
| | CAAAATCACACTGCTGTTCACACCAGAAGTCCCTCCTCTACGATCA[A/G]ATTCCTAATCC | |
| | CAATTTCTACTCACACACCTCGTGGGAGGCCAACACCTTCTTCTGGTTCTTCATTCTCTTCC | |
| | TCCCCAGGGCTGACCATCACCAAAGCCAAACAGCT | |
| D5: rs17705177 | TCAGTTTCCTTCCCCAGAAAATTGTATATCTTGTAGGGTTATTGTGAAGATTAAAGTGGAAT | 69 |
| | GTGCATGCAAAAGTACTTTGCAAACCACAAAGCTCTAGGTTGG[A/T]GTAAATAACTGAAC | |
| | TTTTAAAAAAAATTTACTTTAAGTTCTGGGATACAACGTGCAGAACGTGC | |
| D6: rs358806 | ACTTTCTGGAGGGCAGTTTGGCAATATTTGTCAAATTTTTGAATGTGCGTGGGCTTTGACCG | 70 |
| | AATAACTCTACTCACAAGGATATGTTCTAAAAAGAAAAACACACACGTACATGTGCAGTACA | |
| | AACAGCAAAACTCAATATTCAA[A/C]GTTCAATAAAATTCGTACCACTTTAAAATGATGAG | |
| | C | |
| D7: rs5015480 | GCTCACCCTAGGGAAGTGTTCTTAGGGAAGCATTTCTAATATTTCCAGCTGTCCATATATTT | 71 |
| | TCAAACAAATAATAGGGTATTGAAGTAAACTCGAATGTTGATTATA[C/T]GTTTTCTATCA | |
| | AATTATTCAAGTATTCATTCAGAAAATATTTATTGAGCACCTACAATGTGGC | |
| D8: rs7020996 | CATTGTGGGGGAAAGTCTGTCTTTAGAAAAGAAATGTAAACTGGGCAAGTAGTCTCATCAGT | 72 |
| | TAAATGATTTCCTTGTTGACATAAGGTGAGGAAAAGAAGAA[C/T]AACTTTTGGGAAAAGT | |
| | AACTGTGAGAATACAAGGGAAGAAGAAAAATAAGGGGTTGAACATTGAGGA | |
| D9: rs7659604 | GCAAATGTGTTAGGGTAGAGAACATTTTAATGTTATTATCCTAAAAGGAATCTTTAGACTGA | 73 |
| | TAAAAGCTATGGTATTTAACTGTCATGGCTATAATGGCCTTAGCTATAACTT[C/T]TGAAT | |
| | CTCAGTGGGAATGGTAGGGGAATAACTGTATTGCACAACTGGTAACTTACCTTTTCTGATAT | |
| | TTCTCCAAGAGAGGCTGTTCA | |
| D11: rs2733359 | GAGGGTTGTGACGGTCAACTGTTTTTGTACACATCTTCGATTATTC[C/T]TCCTGTTTTCA | 74 |
| | GCCTCATTCTCTCGTTCTAGGCCATCCTAAAGTACCTGTCATCTCTACGTCTGTGGCCTTCT | |
| | CTGGGCTCCACTAGGCATGTCCCCTTTGCATGTATTCCAAGCTGG | |
| D15: rs4790797 | GGAGCTCTTTGCAAACTGTGAAATTCTGTGTACTTTGAGGGAGAATAATTGTTAATATTTAT | 75 |
| | TAAACATT[A/G]TATTGTATGATTTAACCTTCATAATAATGGTTTTCTATACAGAACCATT | |
| | TTTTTATTCTTGTTTTAGAGGCTGAAGTCTT | |
| D16: rs7223628 | TCATCAGGGAAGAAGAGAGAGAAAGAAATGAAAATAAACACAGCTTGCAGCACATTTGGCAT | 76 |
| | TAACATGAGATCAGCTGCTCTCTGACCCA[C/T]TTCCTCATAGTTGTTTGGTGCCTATTGT | |
| | CTTAGAATCACACTGACCCTAGATTACAGTTTCCCTTAACTGCTCCA | |
| D17: rs8182352 | AACCGTGCTGTCTCAGCATATTGGTCTGTTCCTGCACAACCAAAAGCTGTAACACTTCTGCT | 77 |
| | TTCTCTGGGTTCAGCCCAGCAGAACCATAATGTGGAAATTTCAACTGGGCTGCCTCTGTC | |
| | [C/T]TTGGGCATATGCCTCCTCCTCCGTCAAACACACTG | |
| D18: rs8182354 | TGCAAATGAGATTTGGCTGTAAACCTCTAAACTCATCTCCTTCTGTTCCTTACCTTCTACCT | 78 |
| | TGCTCTTTACTTCTTATCATTCTAAGATAAATTCCC[C/T]TTTAGAGTTTCTGGTCTTGAA | |
| | ATTACCCTTCTATTTTTGCTATATTGCCTGTGGTCTCCCTTTTTAACACCTTGTAAGGCCAC | |
| | ATCTC | |
| D20: rs11761231 | AAGGCATGCAGAGCTTTTGTGTTCAAAGAATTCTGTCTTTTTCCTCCCTAAAGCCATTGCAT | 79 |
| | TTGTTTCAAATCTACGTGTGACTACATTTGGAGATAAGTAGCC[C/T]TTTTCAGACCTTCT | |
| | TGATTTCAAAACACAGATTTGGTCTGCACGTTCTCATGATAAGACAGAGAAGGAGACCATGG | |
| | AAATATTTTGCCTGTCTGTAATTGGCAGGGCTG | |
| D23: rs6920220 | TGCTACGGCAGCGTAACATAGTAGGTGAAGTACCCATTGATAAATTATATTTTATCTGCTTC | 80 |
| | CATCTGTTAGCAGGTAACTTCTCCACTAAAA[A/G]GATATGGTTCTGTAGAACAATGGCAT | |
| | ATGCAGACAGTGATCTGTTATTCCACTATTCTCTTAAGCTATCAATCAGATTGATGAGGCAA | |
| | ATTTATGCTTC | |
| D25: rs6679677 | ATTTTTCAGGTGCCCTGTTGGAAACTATTCAGTGCTTCCTGCGGCTACCAGCGAACAAGGTC | 81 |
| | TGAATCCTTGCTCCCAA[A/C]CAATAATCTGTGATCTTAAGCAATTTATTCAACTAACAAG | |
| | CCTGTTTTCTCACCTGTATTATGGAGATAGTCACCTTCTTAAGGATGTGAGGATTAAATGAG | |
| | AAACCC | |
| D26: rs12141187 | TCAGCATCAGTCACCTCAGCCAGGTCCCTGAATCACAGCCAAGCCTAGATGAGTGGTATTAT | 82 |
| | TGACCATGATAATGGGAGGATGAATGGTGGCTATGACTG[C/T]CTGCTGCAATCAACCTTT | |
| | AGGATGGCCAGAAATTCTGATTTGGCCAGCCCTTGGCCCAGACAGCAATGTCCCCAAGA | |
| D28: rs4132958 | TAGACACAGGCCTGCACAAAGAGCTTGCAATCTATAGATGGATCAGTTGTCATTATATAAAG | 83 |
| | CTCCATATCTTCATTATCAAAAGCAGCTATGCTGAATGC[C/T]CTTCTCTGAAAGATTGTA | |
| | AGCAAGCTCTGCAGAACCTGGGCAGGCCAGGGTGAGCCTTGCTCTGTGGAGATTATAACAGA | |
| | AAATAAAAAATAAAGGAAATGTAGATGGGCATACCAGCTC | |
| D32: rs952477 | GCCTTCATGCCCTGACTTCAGTGGGAGAGAATTAGGCATGGTTGGTAGTGGATTCCCTCTCC | 84 |
| | TTTTCTCCTGTCC[A/G]TGGAGGCTATTGTTCCAAGCCCACCACAAGAGTTCTTAAGCCTG | |
| | GGATCCCAGAAGATTCCATTTGCCTTAAGCC | |
| D33: rs10798269 | TGGACCATTTGAGGTGATGAGCCTGACCCTCTAAAAAAAGGTTAAGCAATTTAATGGGTGAG | 85 |
| | GAAGTTTTTTTGAAGCCTATATCCCCAACCAGTTCCCCAGGGCAG[A/G]TAGATTTGTAAG | |
| | GAGAAAAGGAGGAGAGATTGGTCGACCTCAAGAAATCTAGATATTCTTCAGGTAACAAACAA | |
| | GAAAGCAGACACAGGTGAATGCTTTGGTTTCCCTGGAGGTCTCTC | |
| D35: rs729302 | TGAAGCCCTGCTGAGAAAGTACTGGGTCCCTATTGGAACCCACTCTCTGCACATCTGGAAAT | 86 |
| | CTTTGGAAATAGACCAGAGACCAGGGTGCAGGTGTGCCATGGGACAAGGTGAAGAC[A/C]C | |
| | AGGATCACCTACACACCAGAGTCCACCCAGTAGGA | |
| D36: rs11171739 | GGAGGGACCAATCAACAGTCTTATAAGTAGATACAACAGTGTATAAACAAGGAAACCAAGGA | 87 |
| | AGATTTTTCTC[C/T]TTCAGAACTCGGACCCTGAATACCAGGTTGAGCTGGAGCTGAGTGA | |
| | GTAATAAAATGAAAGGCCCTTTAATGTGGGGGAGGGTAGGTAG | |
| E1: rs7716600 | TGTGAACTTGTATGGCAACCAAAATGATCAATATATGAAGTGAAGTAGGCATAACACTAAGA | 88 |
| | AGAAACTAAAAAACTTATAATGATAGTTGAGTGTGTTAACCCATCTCTTTTGGAAACAGAGT | |
| | AGCAGACAAGAATATTATAGGAAGATGTGCACATGTACC[A/C]CAAAGCTTAAAGTACAAT | |
| | TAAAAAAAAAGAATATTATAGGAAGATGGTGAAAAGGAAGAG | |
| E2: rs11249433 | TTGGAAACATGGATCCAAAACTGTGAAAGAAAAAGCAGAGAAAGCAGGGCTGGGTTTAA[C/ | 89 |
| | T]TTTGGAGTTCCTTGGTTGCTTCTCCTTAGCACAGTGACTCATTTGATATCATCTTTAATT | |
| | TCTCTGGCTAAAGGTTTTCCAACAGATAT | |
| E3: rs3803662 | TTGTCATCCAAAGCACCAACTATGAGAGATATCTATGTGCAATGGTATATAGATCTGTCATA | 90 |
| | GAAGGGTTTAATTATATCTGCCTAATGATTTTCTCTCCTTAATGCCTCTATAGCTGTC[C/T] | |
| | CTTAGCGAAGAATAAAACTGTGGACTGACCCCCACCCATTTGCGAAGAAAGTACTGGGTCT | |
| | TCAGCTTTCATTGTTCAGCCGGTGGTCTTTGTGGACAACACCAGG | |
| E4: rs393152 | CCTACTGCCTTGGAATCTGCTGAAGACCAAGCCCCTGCCCCCAAGCCATGGCAAAGAAGGAG | 91 |
| | GGAAGGAAGCAAAGGTGCCCAGCGGGGACAACTCGGGGAGGGGCGAGGTGCCCAGGGCCCAG | |
| | GAAGGCCAAGCAGCATGTGGCAGGGCAGCATCAGGTGACTCCCAAGAAGGAATGAGGAGAGG | |
| | AT[A/G]TGAGGAAAGAGCCACAGCACAGAGGCCTGCTGTTAGGTCAGCGGAGAC | |
| E5: rs1491923 | TCTGCACCTTTGGCTTTTAGGAATC[C/T]ACTTTGCTCTGGCATTCTCCTAATTTTCTAGA | 92 |
| | AAATTATTGGTCTATTTCATAATTTTATCTTCATTTCCTTAAATCCCAAATATTGATATTTC | |
| | CCAAGGGTTTATTTTTGACACTTTTCCCTTCTTGCTTGAGATCAATGATTCTTAATTAATGT | |
| | GTGTTGGGAAAGAGGG | |
| E6: rs2736098 | CGTGGTTTCTGTGTGGTGTCACCTGCCAGACCCGCCGAAGAAGCCACCTCTTTGGAGGGTGC | 93 |
| | GCTCTCTGGCACGCGCCACTCCCACCCATCCGTGGGCCGCCAGCACCACGC[A/G]GGCCCC | |
| | CCATCCACATCGCGGCCACCACGTCCCTGGGACACGCCTTGTCCCCCGGTGTACGCCGAGAC | |
| | CAAGCACTTCCTCTACTCCTCAGGCGACAAGG | |
| E7: rs801114 | CTCCCCAGTGCATCATTTTCAGTTTTGTCTTTTACTTTCAAAGAAAGCTGTCTTTCTGACAC | 94 |
| | TGCATTCTGCCCTTTCTGACCCA[G/T]GTCCCATATTTAAAGGCTTCACATAGACTATATA | |
| | ATCCAAGTTATCCCTCTGTGGAGAAAGTGGCT | |
| E8: rs2151280 | ACTCGATGGCCCTCAAAAG[C/T]GAAACAAGCTACTATCAGGACCTCTATAGAAAAAGTTT | 95 |
| | GCCAACCTCTACACTGTAGTATGCCTTAAGGATTTTTAGAAGATTGAGTATGATAAACACTT | |
| | TCAAAGAATGATGAAATTCTGAGAAATGGG | |
| E9: rs4636294 | GGGTTGAGCCAGATCTTCAAGACTTAAAAGGATTTAAGTCC[A/G]ATAGTAAAAGGAGCGA | 96 |
| | AGGGAATTCTAGTAAAAGGGAACAGCTTGAGGAATGACCTAGAGACATGACAGTGATCTTTG | |
| | GAGAAATGGCAGTTAGACAGACATTCTGTCTACTCGTTTCCCTGTTACATCCC | |
| E10: rs823128 | ACTGGCTTTGGGTTGTTCACAGT[A/G]GGATACAAATTCCTGCTTCATCTCTTAATAGTTA | 97 |
| | GGTGAACTGTGTAGTTACTTTTTTTATCCTAACCTCAGGCCTAACATATGAAATGAGGATAA | |
| | CATATGCCTTTAAGAGTTGTGCATGATTTTGAAATATGTATAAAGTACCTGGTGGAATTATT | |
| | TGGCATCT | |
| E11: rs947211 | AAAGGCCAGGGAAAGAAGACAGGAAAAAAGTGAAAACTAAAGAGAAAATTTTGCTTCA[A/G] | 98 |
| | AGAACTGGTTGTGTGGTTCCCAACTGTCCATATGGCACAGGAAAGTCTCATCTGTGAAACA | |
| | AAATAAAGTTCCCTTCCAACACAGACATGACTGTTCTAATTTCCTATGTTATTTCAACTCTC | |
| | TAGGAGGTGAGAAAAGCAGAAATTATTGCACCCTAGGCCAT | |
| E12: rs2736990 | ATGTCTGCCTTTGCATCAGATAATGGCTTACAAGTTAATCTCCTCTTGCTCCCTGTTACACA | 99 |
| | CATATACA[C/T]CTTCTTCCTAAACAGCTCATAAGGTGAAAGAAAGACTCAGATTTCTGAC | |
| | TATGTAATTGATAATATCACACGGACTGCCTGCTCATCATCTGCTAGTCACATTGGCAGAGT | |
| | TGACAG | |
| E13: rs12418451 | GTAAGGGAGTGCTGCTCCTGGACCTGCTCCTGAGAATGGCTCCTGGGAGTGATGTAGGTGAC | 100 |
| | TGATTGATGGGGTGGGACGAAGCTGGGCAGAGGCTTGGGTAGCTGGGACTGTAACAGTTATG | |
| | TGAGAGGAAGCGGGAATCTGAGAGAGTTGCC[A/G]GGGCAAAATGTAGGCCCCCAGCCCCT | |
| | GGTTCAGGGGACAGCCCAGGGATAGTCACCAGGGATCCAGCGATGTGTGTGTGT | |
| E14: rs10896449 | AGCAGAATGTGGAAGGATGGGCAGGAGTTGTCTAAGAGAAGAGTGTGGCAATAGAAGGGCAC | 101 |
| | CCTGGGCCACAGGGAACAAACCATAGCTGAAAGATGAGGAGTCAAGAAATATTCTGGCACCC | |
| | ATGGGGTACTATTAGCAGTTTAACTTTACAGGAGCTGAAA[A/G]TTTAAGAAGGGGAATGT | |
| | CAAGAGATGAGGCTGAACCTTGG | |

TABLE 2
Primer sequences(Forward -F; and Reverse-R)

| | PRIMER SEQUENCE | | Expected |
|---|---|---|---|
| | (FORWARD -F; AND | SEQ ID | Product |
| Name/SNP | REVERSE-R) | NO: | Size |
| rs2670660 | F: CCACGCACAAGTGATCTACC | 102 | 152 |
| | R: CAAGATGCCTCTATGCCTTAAA | 103 | |
| A1: rs6458307 | A1F: TCTTTAATACAGATTGGGAAGAGG | 104 | 150 |
| | A1R: AACTTTCAACTGCCAGGACA | 105 | |
| A2: rs9472138 | A2F: ACAGTTGTGCAACCATCAGC | 106 | 165 |
| | A2R: GACTTTCTGGAAAAGGCAAAA | 107 | |
| A3: rs6596075 | A3F: TTGTGTTCAAGCCTCCTTCC | 108 | 171 |
| | A3R: TCTGAGCTTAGCCTCCCTGA | 109 | |
| A4: rs2544677 | A4F: GGAAAACACTGGGAGGGAAT | 110 | 178 |
| | A4R: CCTGGGTGACAGAGGAAGAC | 111 | |
| A5: rs6983561 | A5F: GGTTCTGTGAAGCGGGTAAA | 112 | 177 |
| | A5R: TCATGGACCACAAATTTCCA | 113 | |
| A6: rs16901979 | A6F: GTGGGGTCTTTGTTGTGGAG | 114 | 188 |
| | A6R: TGTTCAGAGCGGTTGAATGA | 115 | |
| A7: rs672888 | A7F: GCCATGTCTAACTGGGCATT | 116 | 153 |
| | A7R: GCTGAGTGATGCTGGCAATA | 117 | |
| A8: rs13281615 | A8F: GACACGTGGAATTTACTCTTTTGA | 118 | 168 |
| | A8R: GCCAAGCCTACACTTCCTCTT | 119 | |
| A9: rs10505477 | A9F: CCGTGGGAAACAAAGTCTTC | 120 | 185 |
| | A9R: TTCCAACCTGAAACACACACA | 121 | |
| A10: rs10808556 | A10F: CTCCATAGAGCCTGCAGAGG | 122 | 211 |
| | A10R: TTATTCGTCCCTCTGTTTTATGG | 123 | |
| A11: rs6983267 | A11F: TCCTTTGAGCTCAGCAGATG | 124 | 154 |
| | A11R: TGAGAAACTTGCTGGGTTCC | 125 | |
| A12: rs7014346 | A12F: GCTTGCAGCTTCTGCCTAAT | 126 | 160 |
| | A12R: AACTTTTGGGGAGGCTGTTT | 127 | |
| A13: rs7000448 | A13F: AGGCTCCTTAGGGAAGGTGA | 128 | 165 |
| | A13R: GAGATTGTGCCACTGCACTC | 129 | |
| A14: rs1447295 | A14F: GAGTTGCACGCCAGACACTA | 130 | 173 |
| | A14R: AGGGGTTCCTGTTGCTTTTT | 131 | |
| A15: rs2820037 | A15F: AGTGATTGCTCTAATTGCCAAG | 132 | 191 |
| | A15R: GCGCATGAGGTCTATGTTGA | 133 | |
| A16: rs889312 | A16F: GGCCATCTGTTTTACCAACC | 134 | 151 |
| | A16R: TGGGAAGGAGTCGTTGAGTT | 135 | |
| A17: rs1937506 | A17F: CGGGAAAGTAAAAATTGTTATCTCATT | 136 | 156 |
| | A17R: GAGGACCAATCCTTTGGACA | 137 | |
| A18: rs4242382 | A18F: AAAGAGGTAACCCAGGGAACA | 138 | 151 |
| | A18R: CATAAGCCTTCGCTGACTCC | 139 | |
| A19: rs7017300 | A19F: TGAGCCAGGACATCAGAAAG | 140 | 189 |
| | A19R: CCATCCCTGTGAGTCATCCT | 141 | |
| A20: rs10090154 | A20F: TTCTCTCCAGATTGATACACAGC | 142 | 166 |
| | A20R: AATGCCTCTGCCAATACCAC | 143 | |
| A21: rs7837688 | A21F: TCACAGGAAAATTGAGCAGAAA | 144 | 178 |
| | A21R: ATGTGCAATGCCAAGAATGA | 145 | |
| A22: rs2542151 | A22F: GTAGCCCCACTTCGCCAAT | 146 | 116 |
| | A22R: TCCCTATCGCAGAGGAAAAA | 147 | |
| A23: rs16892766 | A23F: AACGGTCAGACGCAAACAGT | 148 | 196 |
| | A23R: GGCAGCTCCTCATTCCTAAA | 149 | |
| A24: rs6997709 | A24F: GACCAAATTGAAGAATTGGTTTG | 150 | 174 |
| | A24R: ACTTGAGCTCGATCCACAGC | 151 | |
| A25: rs6457617 | A25F: TCAATCCCCATATGCACAGA | 152 | 153 |
| | A25R: ATGACATGCTCTCACGATGG | 153 | |
| A26: rs9469220 | A26F: TGGCAGTCCAAGCTACTAAGAA | 154 | 177 |
| | A26R: TGCTGCATGGTAAATTTTTG | 155 | |
| A27: rs660895 | A27F: GGGAAACGAAGGATGAAAGA | 156 | 123 |
| | A27R: TTCCTGGTTGATTTCCCTTC | 157 | |
| A28: rs615672 | A28F: CCATGAGCCTATCACACTCG | 158 | 154 |
| | A28R: TGCCGATATTTCCGATTTTC | 159 | |
| A29: rs9270986 | A29F: ATGTTCATCAGTGGTCACAAATA | 160 | 123 |
| | A29R: GGCTCATGGTAGGTCGTCAT | 161 | |
| B1: rs10186922 | B1F: AGCTCTGACTCCCAACTCCA | 162 | 236 |
| | B1R: CGACAGATGGCTACAAAGCA | 163 | |
| B2: rs11159647 | B2F: GCTCACTACCTGGGTGCAAT | 164 | 166 |
| | B2R: TTGTCAGCATTTTTCAGATTTC | 165 | |
| B3: rs2609653 | B3F: TGTGCACAAGAGCATTGTTTT | 166 | 203 |
| | B3R: CCAGGATCATCCATGTTGTG | 167 | |
| B4: rs7570682 | B4F: GAGTGATGGAGTGGCATAGG | 168 | 213 |
| | B4R: AACCCCCTACATGCTTCCTT | 169 | |
| B5: rs13387042 | B5F: CCCTGTTTTGTTGCAGTGAA | 170 | 172 |
| | B5R: ACGGAGCACTCTCAACATCC | 171 | |
| B6: rs2291533 | B6F: CAGAAGCAGCAGCAGGTACA | 172 | 158 |
| | B6R: AAGCTACTGGCCCAAAGACA | 173 | |
| B7: rs2822558 | B7F: TATCGACAAAAGTTTTCCACTG | 174 | 157 |
| | B7R: CCCTGCTAACACTGCTGGAC | 175 | |
| B8: rs10795668 | B8F: GGCATTGCGTTCATTCTGA | 176 | 215 |
| | B8R: AGCGAGACTCCGTCTGAAAA | 177 | |
| B9: rs4779584 | B9F: AGCTGCTATAAGATGGGCTGA | 178 | 181 |
| | B9R: TGCCACTGCTAAAAGCCATT | 179 | |
| B10: rs10757274 | B10F: GTTTCTGCACATGGTGATGG | 180 | 250 |
| | B10R: CTGCCTCACTCTCCAGTTCC | 181 | |
| B11: rs10757278 | B11F: CAAACAGCCAATTTGTGGAG | 182 | 182 |
| | B11R: GGCGTTACAATTAAAGAGAGAGAGA | 183 | |
| B12: rs1333049 | B12F: TCTGCTTCATATTCCAACTTGTG | 184 | 182 |
| | B12R: TGCTTCTGCATACTTTTGTCAAC | 185 | |
| B13: rs2383206 | B13F: GGCCCGATGATTTTCAGTTA | 186 | 170 |
| | B13R: GACATAGCTCTACAGCTGGGAAT | 187 | |
| B14: rs2383207 | B14F: ACTTAGCCCTTGGGACCATT | 188 | 156 |
| | B14R: AAGAAGCTAAGAGAATGTTGAGCA | 189 | |
| B15: rs383830 | B15F: GACCCCTGATGTAAACTACTCTTTG | 190 | 193 |
| | B15R: GCTGGTGGGTTTCTGTAGGA | 191 | |
| C1: rs7250581 | C1F: CTCCAAAAGCCAGGAGAATG | 192 | 214 |
| | C1R: CCCGTGTGGCTGCATATTA | 193 | |
| C2: rs10733113 | C2F: CACAGTCTGTTACAAGGGTGGA | 194 | 187 |
| | C2R: TACTTCTTGCGGCCTGTCTT | 195 | |
| C3: rs10761659 | C3F: GGATTCTTCGCATGATGAGG | 196 | 244 |
| | C3R: AGTCAAAGAGGAGGGCGTTT | 197 | |
| C4: rs10883365 | C4F: GAAGGCCGCATAAGACGTTA | 198 | 235 |
| | C4R: CGTGTCTCTTCCAGCGCTAT | 199 | |
| C5: rs17234657 | C5F: AGTGCTGAAGCGGAATTGAG | 200 | 215 |
| | C5R: ATGAGCAGCAATGGTCACAG | 201 | |
| C6: rs55646866 | C6F: AGAGTCCTCAGCCTCGTCAG | 202 | 243 |
| | C6R: CGAGAAAGCAAGCTTCAGGT | 203 | |
| C7: rs6672995 | C7F: AGGGTTCCTGGCTCCTACAG | 204 | 190 |
| | C7R: CAGAGGGTTGGGTTCCAGTA | 205 | |
| C8: ss107635144 | C8F: GCGTGGTGAGGTGATTACTG | 206 | 165 |
| | C8R: AAGAAAACACAAGTGAGGCACA | 207 | |
| C9: rs12037606 | C9F: CTGGCAGAGGATTTGAGACA | 208 | 173 |
| | C9R: AGGTAGCTCAGCTGTTCATGG | 209 | |
| C10: rs6601764 | C10F: ACCAGTGGTCCAACCCACTA | 210 | 221 |
| | C10R: TCACCATCTGGAAGGCTTTT | 211 | |
| C11: rs7807268 | C11F: GGAGGACAGGTTGGAGAACA | 212 | 190 |
| | C11R: AAGGGACTGGAAGGGTGATT | 213 | |
| C12: rs6957669 | C12F: CTAGGCGTTTGCATTCATCC | 214 | 223 |
| | C12R: TGACGCAAAGACTGAAAGGA | 215 | |
| C13: rs12970134 | C13F: GGTGGTGATTACTGCCCTTG | 216 | 203 |
| | C13R: CAGTGTGGAGACATGCTTGC | 217 | |
| C14: rs17782313 | C14F: CTTGGAAGCAGGAAAACCAG | 218 | 180 |
| | C14R: GCTACCTCAATCCCAGATGC | 219 | |
| C15: rs1859962 | C15F: CCCGGAAGGCAAATAACAAT | 220 | 166 |
| | C15R: TTGGGAAATTTAGCCCCATT | 221 | |
| C16: rs983085 | C16F: GGAATTGTACACCATCACCAAA | 222 | 154 |
| | C16R: TTTGTCAAATGCTTTTTCTCCA | 223 | |
| D1: rs10490072 | D1F: TGCAAGCTCCAAGAGAGTGA | 224 | 174 |
| | D1R: AGGCCCGTGTCCTGTAATAA | 225 | |
| D2: rs1153188 | D2F: GAAGATGGTCTGAATGGCAAA | 226 | 150 |
| | D2R: TGTTTCGTCCACTGGATCTG | 227 | |
| D3: rs13071168 | D3F: CCCACATCCAGACTTCTGCT | 228 | 217 |
| | D3R: AGCTGTTTGGCTTTGGTGAT | 229 | |
| D4: rs17036101 | D4F: ATTAGGGGCCAGGAAAGAAA | 230 | 213 |
| | D4R: TGCCTGGCATTTAAAAATCT | 231 | |
| D5: rs17705177 | D5F: TCAGTTTCCTTCCCCAGAAA | 232 | 170 |
| | D5R: GCACGTTCTGCACGTTGTAT | 233 | |
| D6: rs358806 | D6F: ACTTTCTGGAGGGCAGTTTG | 234 | 183 |
| | D6R: GCTCATCATTTTAAAGTGGTACGAA | 235 | |
| D7: rs5015480 | D7F: GCTCACCCTAGGGAAGTGTTC | 236 | 172 |
| | D7R: GCCACATTGTAGGTGCTCAA | 237 | |
| D8: rs7020996 | D8F: CATTGTGGGGGAAAGTCTGT | 238 | 171 |
| | D8R: TCCTCAATGTTCAACCCCTTA | 239 | |
| D9: rs7659604 | D9F: GCAAATGTGTTAGGGTAGAGAACA | 240 | 203 |
| | D9R: TGAACAGCCTCTCTTGGAGAA | 241 | |
| D10: rs2716914 | D10F: CGAACCAGAGGGCATAAGAG | 242 | 150 |
| | D10R: CAAGATCATGGGCTTCACAA | 243 | |
| D11: rs2733359 | D11F: GAGGGTTGTGACGGTCAACT | 244 | 165 |
| | D11R: CCAGCTTGGAATACATGCAA | 245 | |
| D12: rs35658367 | D12F: GAAGAATTTGGGCAGTGAGC | 246 | 199 |
| | D12R: ATCCATGGCCATTCATTCAT | 247 | |
| D13: rs3926687 | D13F: GGCAAGGAGGCAGAACAGT | 248 | 150 |
| | D13R: GGGGGAAATGAATTGTCAAA | 249 | |
| D14: rs4790796 | D14F: AGGTGGTGATGGTTTTGTCC | 250 | 205 |
| | D14R: AAGACTTCAGCCTCTAAAACAAGAA | 251 | |
| D15: rs4790797 | D15F: GGAGCTCTTTGCAAACTGTG | 252 | 151 |
| | D15R: AAGACTTCAGCCTCTAAAACAAGAA | 253 | |
| D16: rs7223628 | D16F: TCATCAGGGAAGAAGAGAGAGAA | 254 | 167 |
| | D16R: TGGAGCAGTTAAGGGAAACTGT | 255 | |
| D17: rs8182352 | D17F: AACCGTGCTGTCTCAGCATA | 256 | 158 |
| | D17R: CAGTGTGTTTGACGGAGGAG | 257 | |
| D18: rs8182354 | D18F: TGCAAATGAGATTTGGCTGT | 258 | 187 |
| | D18R: GAGATGTGGCCTTACAAGGTG | 259 | |
| D19: rs878329 | D19F: TCCACTCAACTCCCTCAACC | 260 | 150 |
| | D19R: AGCCAAGTTCTTGGATCTGC | 261 | |
| D20: rs11761231 | D20F: AAGGCATGCAGAGCTTTTGT | 262 | 215 |
| | D20R: CAGCCCTGCCAATTACAGAC | 263 | |
| D21: rs11162922 | D21F: TTTGTTGATATCTTCTTGTTTGGTA | 264 | 213 |
| | D21R: CATGGGGAGAGAAAATACTCTGA | 265 | |
| D22: rs2837960 | D22F: TGTTGCTGAGACCCTCAGTG | 266 | 177 |
| | D22R: AGTCAAGCAGTAGCCCAGGA | 267 | |
| D23: rs6920220 | D23F: TGCTACGGCAGCGTAACATA | 268 | 193 |
| | D23R: GAAGCATAAATTTGCCTCATCA | 269 | |
| D24: rs743777 | D24F: GCCTCCTGTGCTTTCTCACT | 270 | 170 |
| | D24R: GCCTCAGAGAGAATCGGATG | 271 | |
| D25: rs6679677 | D25F: ATTTTTCAGGTGCCCTGTTG | 272 | 188 |
| | D25R: GGGTTTCTCATTTAATCCTCACA | 273 | |
| D26: rs12141187 | D26F: TCAGCATCAGTCACCTCAGC | 274 | 179 |
| | D26R: TCTTGGGGACATTGCTG | 275 | |
| D27: rs2644577 | D27F: AATCTGGGCATAGCCAACAG | 276 | 166 |
| | D27R: AGGCAAGGAGGGTTGTTCTT | 277 | |
| D28: rs4132958 | D28F: TAGACACAGGCCTGCACAAA | 278 | 222 |
| | D28R: GAGCTGGTATGCCCATCTACA | 279 | |
| D29: rs4950437 | D29F: TTTTTAATGCCCCATGAATATG | 280 | 103 |
| | D29R: GGTTTCTGAGGTTGCACACA | 281 | |
| D30: rs6684174 | D30F: CCAGAGTGGAATCAGCAGGT | 282 | 234 |
| | D30R: CGGCGCAGACTTTCTTTTAT | 283 | |
| D31: rs8029320 | D31F: TGCATAAGCCAATTCCTTGC | 284 | 209 |
| | D31R: AAATCGTTTGCTTGGGTGAG | 285 | |
| D32: rs952477 | D32F: GCCTTCATGCCCTGACTTC | 286 | 151 |
| | D32R: GGCTTAAGGCAAATGGAATC | 287 | |
| D33: rs10798269 | D33F: TGGACCATTTGAGGTGATGA | 288 | 227 |
| | D33R: GAGAGACCTCCAGGGAAACC | 289 | |
| D34: rs12537284 | D34F: AGGTTGCAGTGAGCCAAGAT | 290 | 243 |
| | D34R: AATACGTAAGCGTGGGGTTG | 291 | |
| D35: rs729302 | D35F: TGAAGCCCTGCTGAGAAAGT | 292 | 155 |
| | D35R: TCCTACTGGGTGGACTCTGG | 293 | |
| D36: rs11171739 | D36F: GGAGGGACCAATCAACAGTC | 294 | 163 |
| | D36R: CTACCTACCCTCCCCCACAT | 295 | |
| D37: rs11052552 | D37F: TCCCTTAAGGCATAAGACAGC | 296 | 241 |
| | D37R: TGAGGCTGCAGTGAGCTATG | 297 | |
| E1: rs7716600 | E1F: TGTGAACTTGTATGGCAACCA | 298 | 223 |
| | E1R: TCTTCCTTTTCACCATCTTCC | 299 | |
| E2: rs11249433 | E2F: TTGGAAACATGGAATCCAAAA | 300 | 150 |
| | E2R: ATATCTGTTGGAAAACCTTTAGCC | 301 | |
| E3: rs3803662 | E3F: TTGTCATCCAAAGCACCAAC | 302 | 227 |
| | E3R: CCTGGTGTTGTCCACAAAGA | 303 | |
| E4: rs393152 | E4F: CCTACTGCCTTGGAATCTGC | 304 | 237 |
| | E4R: GTCTCCGCTGACCTAACAGC | 305 | |
| E5: rs1491923 | E5F: CTGCACCTTTGGCTTTTAGG | 306 | 197 |
| | E5R: CCCTCTTTCCCAACACACAT | 307 | |
| E6: rs2736098 | E6F: CGTGGTTTCTGTGTGGTGTC | 308 | 190 |
| | E6R: CCTTGTCGCCTGAGGAGTAG | 309 | |
| E7: rs801114 | E7F: CTCCCCAGTGCATCATTTTC | 310 | 152 |
| | E7R: AGCCACTTTCTCCACAGAGG | 311 | |
| E8: rs2151280 | E8F: ACTCGATGGCCCTCAAAAG | 312 | 150 |
| | E8R: CCCATTTCTCAGAATTTCATCA | 313 | |
| E9: rs4636294 | E9F: GGGTTGAGCCAGATCTTCAA | 314 | 173 |
| | E9R: GGGATGTAACAGGGAAACGA | 315 | |
| E10: rs823128 | E10F: ACTGGCTTTGGGTTGTTCAC | 316 | 190 |
| | E10R: AGATGCCAAATAATTCCACCA | 317 | |
| E11: rs947211 | E11F: AAAGGCCAGGGAAAGAAGAC | 318 | 224 |
| | E11R: ATGGCCTATGGGTGCAATAA | 319 | |
| E12: rs2736990 | E12F: ATGTCTGCCTTTGCATCAGA | 320 | 214 |
| | E12R: CTGTCAACTCTGCCAATGTGA | 321 | |
| E13: rs12418451 | E13F: GTAAGGGAGTGCTGCTCCTG | 322 | 236 |
| | E13R: ACACACACACATCGCTGGAT | 323 | |
| E14: rs10896449 | E14F: AGCAGAATGTGGAAGGATGG | 324 | 205 |
| | E14R: CCAAGGTTCAGCCTCATCTC | 325 | |
| rs2670660_1 | F: CACGCACAAGTGATCTACCAG | 326 | 110 |
| | R: GCATCAGGATGCACCAGTC | 327 | |
| rs2670660_3 | F: CCACGCACAAGTGATCTACC | 328 | 205 |
| | R: TCCCCTTACATCTGCCACTT | 329 | |
| rs2670660_4 | F: GTGTTCAGGAGCTGGGTGAC | 330 | 225 |
| | R: TCCCCTTACATCTGCCACTT | 331 | |

### Methods of Use

The invention provides methods and reagents for the detection of specific snpRNAs in a biological sample from a subject. In one embodiment, the invention provides primers that can be used in an RT-PCR-based assay to identify the presence of one or more snpRNAs in a sample. The invention also provides probes, in the form of cDNA molecules of the snpRNAs, for use in detecting the snpRNAs in a sample, and allelic variants thereof. The invention also provides diagnostic and prognostic methods based on the detection of the snpRNAs.

Preferably, the presence of a particular allelic variant of the snpRNA is detected according to the methods of the invention. In a specific embodiment, the allelic variant is the A-allele, the G-allele, the C-allele, or the T-allele, denoted with respect to the SNP sequence. In one embodiment, the allele is the pathological allele of the SNP. In another embodiment the allele is the ancestral allele of the SNP.

In a specific embodiment, the pathological allele is selected from the G-allele of rs2670660 or the A-allele of rs16901979.

An snpRNA molecule of the invention is an RNA molecule transcribed from a genomic sequence containing a disease-linked SNP. Thus, the snpRNA can be transcribed from either allele, or from both alleles, of the SNP-bearing genomic sequence. In accordance with the invention, the detection of an snpRNA molecule transcribed from the pathological allele of the SNP indicates an increased risk for the disease or disorder linked to the SNP. The risk is based upon the risk associated with the specific allele of the SNP.

In certain embodiments, the presence of an snpRNA transcribed from a pathological allele translates to an increased risk of developing the disease or disorder or an increased risk of having a more severe or refractory form of the disease or disorder. Likewise, the failure to detect an snpRNA transcribed from a pathological allele, or the detection of an snpRNA transcribed from an ancestral allele, indicates a decreased risk for the disease or disorder. In this context, the term “refractory” describes patients treated with a currently available therapy for a disease or disorder, wherein the treatment with the currently available therapy is not clinically adequate either (i) to relieve one or more symptoms associated with the disease or disorder, (ii) to stop or adequately slow the progression of the disease or disorder, or (iii) to resolve the pathological effects of the disease or disorder.

The methods of the present invention, because they are based upon the detection of snpRNA molecules, and allelic variants thereof, offer an improvement over methods based on the detection of the SNPs themselves. This is because, according to the present invention, the SNP itself is not functional and its mere presence, like that of a gene, does not necessarily have a biological consequence. Rather, the biological consequence results from its transcription, in this case into a non-coding regulatory RNA molecule.

The invention provides methods for detecting an snpRNA molecule in a sample. In a preferred embodiment, the sample comprises the fraction of small RNA molecules from a cell or tissue. Preferably the fraction of small RNA molecules is substantially free of contaminating DNA molecules and protein.

In one embodiment, the method comprises contacting the sample with one or more short (10-30 base pairs) oligonucleotides under conditions permitting the hybridization of the one or more short oligonucleotides with the snpRNA molecule or a corresponding cDNA thereof. In accordance with this embodiment, the method further comprises one or more rounds of a polymerase chain reaction (“PCR”) after the contacting step. In one embodiment, a step of reverse transcription precedes the contacting step. In one embodiment, the PCR reaction is a nested PCR reaction. In one embodiment, the method further comprises the step of visualizing the PCR products of the PCR reaction using gel electrophoresis with or without an additional step comprising Southern hybridization. In accordance with this embodiment, the snpRNA molecule is detected in the sample if a PCR product of the predicted size is amplified in the PCR reaction. In one embodiment, the oligonucleotides are labeled with a detectable label.

In another embodiment, the method comprises contacting the sample with one or more longer oligonucleotides (50-300 base pairs) under conditions permitting the hybridization of the oligonucleotides with the snpRNA molecule or a corresponding cDNA thereof. In one embodiment, the oligonucleotides are labeled with a detectable label. In one embodiment, the sample is bound to a solid support. In a specific embodiment, the solid support is a bead or a membrane support. In accordance with this embodiment, the snpRNA molecule is detected in the sample if the oligonucleotide selectively hybridizes with a molecule of the predicted size. Selective hybridization is determined using methods routine in the art of nucleic acid hybridization assays. For example, increasing the salt content of the wash buffers and the number, length, and temperature of the washing steps increases the specificity of binding.

The invention provides methods for determining the likelihood that a human subject will develop a disease or condition linked to an SNP by detecting the presence of an SNP sequence-bearing RNA molecule in a sample from the subject. In accordance with this embodiment, the subject has an increased likelihood of developing the disease or condition where an snpRNA transcribed from a pathological allele of the SNP is detected in a sample from the subject. Likewise, the subject has a decreased likelihood of developing the disease or condition where either no snpRNA is detected in the sample or an snpRNA transcribed from an ancestral allele is detected in the sample.

In one embodiment, the invention provides a method for determining the risk to a subject of developing a particular disease or disorder, wherein a risk of developing the disease or disorder has been associated with an SNP, the method comprising detecting a small RNA containing the SNP in a sample from the subject by (1) obtaining a biological sample from the subject; (2) extracting the population of small RNAs from the sample; and (3) performing a reverse transcription polymerase chain reaction (RT-PCR) on the extract of small RNA from the sample, wherein the PCR is performed with a set of primers designed to amplify a complementary DNA fragment (cDNA) corresponding to the genomic region containing the SNP. In specific embodiments, the primers are designed to amplify a cDNA fragment that is either sense or antisense with respect to the genomic DNA containing the SNP. In certain embodiments, more than one set of primers is used to amplify the cDNA, wherein the more than one set of primers includes a set of nested PCR primers. In certain embodiments, the more than one set of primers includes a set of primers to amplify the antisense cDNA fragment and the sense cDNA fragment.

In particular embodiments of the methods of the invention, the sample is a cell or tissue sample, a tumor tissue sample, a blood sample, or the sample comprises or is enriched for peripheral blood mononuclear cells (PBMC). It is understood that the embodiment in which the sample is “a cell” includes a plurality a cells. In one embodiment, the cells are a line of immortalized cells. In another embodiment the cells are primary cells which have been cultured for a period of time to increase their cell number. In each of these embodiments “a cell” or a plurality of cells refers to cells which are outside of a body, i.e., cells in vitro.

In one embodiment of the claimed methods, the presence of the G-allele snpRNA of rs2670660 is detected in a sample from the subject, wherein the presence of the G-allele snpRNA indicates that the subject is at an increased risk for developing an autoimmune disorder. In one embodiment, the autoimmune disorder is selected from the group consisting of vitiligo, ankylosing spondylitis, rheumatoid arthritis, multiple sclerosis, systemic lupus erythematosus and autoimmune thyroid disease.

In one embodiment of the claimed methods, the presence of the A-allele snpRNA of rs16901979 is detected in a sample from the subject, wherein the presence of the A-allele snpRNA indicates that the subject is at an increased risk for developing a cancer of epithelia origin. In one embodiment, the cancer is selected from breast cancer, metastatic breast cancer, prostate cancer, and metastatic prostate cancer. In a preferred embodiment, the cancer is prostate cancer or metastatic prostate cancer.

In one embodiment of the claimed methods, the presence of the C-allele snpRNA of rs6596075 is detected in a sample from the subject, wherein the presence of the C-allele snpRNA indicates that the subject is at an increased risk for developing Crohn's disease.

In one embodiment of the claimed methods, the presence of the C-allele snpRNA of rs6983561 is detected in a sample from the subject, wherein the presence of the C-allele snpRNA indicates that the subject is at an increased risk for developing prostate cancer.

In one embodiment of the claimed methods, the presence of the G-allele snpRNA of rs13281615 is detected in a sample from the subject, wherein the presence of the G-allele snpRNA indicates that the subject is at an increased risk for developing breast cancer.

In one embodiment of the claimed methods, the presence of the T-allele snpRNA of rs10505477 is detected in a sample from the subject, wherein the presence of the T-allele snpRNA indicates that the subject is at an increased risk for developing colorectal or prostate cancer.

In one embodiment of the claimed methods, the presence of the C-allele snpRNA of rs10808556 is detected in a sample from the subject, wherein the presence of the C-allele snpRNA indicates that the subject is at an increased risk for developing colorectal or prostate cancer.

In one embodiment of the claimed methods, the presence of the G-allele snpRNA of rs6983267 is detected in a sample from the subject, wherein the presence of the G-allele snpRNA indicates that the subject is at an increased risk for developing colorectal or prostate cancer.

In one embodiment of the claimed methods, the presence of the A-allele snpRNA of rs7014346 is detected in a sample from the subject, wherein the presence of the A-allele snpRNA indicates that the subject is at an increased risk for developing colorectal cancer.

In one embodiment of the claimed methods, the presence of the T-allele snpRNA of rs7000448 is detected in a sample from the subject, wherein the presence of the T-allele snpRNA indicates that the subject is at an increased risk for developing prostate cancer.

In one embodiment of the claimed methods, the presence of the A-allele snpRNA of rs1447295 is detected in a sample from the subject, wherein the presence of the A-allele snpRNA indicates that the subject is at an increased risk for developing prostate cancer.

In one embodiment of the claimed methods, the presence of the T-allele snpRNA of rs2820037 is detected in a sample from the subject, wherein the presence of the T-allele snpRNA indicates that the subject is at an increased risk for developing hypertension.

In one embodiment of the claimed methods, the presence of the C-allele snpRNA of rs889312 is detected in a sample from the subject, wherein the presence of the C-allele snpRNA indicates that the subject is at an increased risk for developing breast cancer.

In one embodiment of the claimed methods, the presence of the A-allele snpRNA of rs1937506 is detected in a sample from the subject, wherein the presence of the A-allele snpRNA indicates that the subject is at an increased risk for developing hypertension.

In one embodiment of the claimed methods, the presence of the A-allele snpRNA of rs13387042 is detected in a sample from the subject, wherein the presence of the A-allele snpRNA indicates that the subject is at an increased risk for developing breast cancer.

In one embodiment of the claimed methods, the presence of the A-allele snpRNA of rs7716600 is detected in a sample from the subject, wherein the presence of the A-allele snpRNA indicates that the subject is at an increased risk for developing breast cancer.

In one embodiment of the claimed methods, the presence of the C-allele snpRNA of rs11249433 is detected in a sample from the subject, wherein the presence of the C-allele snpRNA indicates that the subject is at an increased risk for developing breast cancer.

In one embodiment of the claimed methods, the presence of the T-allele snpRNA of rs3803662 is detected in a sample from the subject, wherein the presence of the T-allele snpRNA indicates that the subject is at an increased risk for developing breast cancer.

In accordance with the methods of the invention, the table below lists the pathological allele of a number of exemplary SNPs which encode an snpRNA molecule of the invention.

TABLE 3
Selected examples of pathological alleles
and the associated disease or disorder

| SNP | Pathological Allele | Associated Disease/Disorder |
|---|---|---|
| rs2670660 | G allele | Autoimmune disorders |
| A3: rs6596075 | C allele | Crohn's disease |
| A5: rs6983561 | C allele | Prostate cancer |
| A6: rs16901979 | A allele | Prostate Cancer |
| A8: rs13281615 | G allele | Breast Cancer |
| A9: rs10505477 | T allele | Colorectal and Prostate Cancer |
| A10: rs10808556 | C allele | Colorectal and Prostate Cancer |
| A11: rs6983267 | G allele | Prostate and Colorectal Cancers |
| A12: rs7014346 | A allele | Colorectal Cancers |
| A13: rs7000448 | T allele | Prostate cancer |
| A14: rs1447295 | A allele | Prostate cancer |
| A15: rs2820037 | T allele | Hypertension |
| A16: rs889312 | C allele | Breast Cancer |
| A17: rs1937506 | A allele | Hypertension |
| B5: rs13387042 | A allele | Breast Cancer |
| E1: rs7716600 | A allele | Breast Cancer |
| E2: rs11249433 | C allele | Breast Cancer |
| E3: rs3803662 | T allele | Breast Cancer |

### Examples

The following examples describe the identification of small non-coding RNAs of the invention (snpRNAs) and the biological activity of specific examples of these snpRNAs.

1.1 Meta-Analysis of Disease-Linked SNPs Reveals that the Majority Occur within Non-Coding Genomic Regions

To assess the genomic distribution of disease-linked SNPs, a meta-analysis was carried out using SNPs identified in several genome-wide association studies. See Glinskii et al., Cell Cycle 2009 December; 8(23):3925-42. The data set consisted of up to 712,253 samples (comprising 221,158 disease cases, 322,862 controls, and 168,233 case/control subjects of obesity GWAS). This analysis revealed that 39% of SNPs associated with 22 common human disorders are located within intergenic regions and 29% within introns. Thus, a majority of disease-linked SNPs identified to date are located within introns (29%) or intergenic (39%) regions of the human genome having no direct relation either to known protein-coding sequences or to known non-coding RNA sequences such as miRNA or liRNA sequences. These data are summarized in the table below.

Chromatin-state maps based on H3K4me3-H3K36me3 signatures show that many intergenic disease-linked SNPs are located within the boundaries of the K4-K36 domains indicating that these intergenic SNP-harboring genomic regions are transcribed, even though none are located within the boundaries of exons of genomic sequences encoding long non-coding RNAs identified to date. The following data demonstrate that these SNP-containing intergenic regions are in fact transcribed to produce non-coding RNA molecules having gene regulatory activity.

TABLE 4
SNP classes defined by analysis of genomic coordinates of disease-
linked SNPs identified in genome-wide association studies of
22 common human disorders. Five intergenic SNPs are associated
with multiple diseases (3 with 3; and 2 with 2); 4 intronic
SNPs are associated with 2 different diseases; 4 missense SNPs
are associated with 2 different diseases.

| | Number of significant | |
|---|---|---|
| SNP class | association calls | Percent |
| cds-synon | 5 | 1.805 |
| missense | 72 | 25.99 |
| UTR-3 | 3 | 1.083 |
| nearGene-3 | 9 | 3.249 |
| nearGene-5 | 4 | 1.444 |
| Intergenic | 107 | 38.63 |
| Intronic | 77 | 27.8 |
| Total | 277 | 100 |
| SNP class | Number of unique SNPs | Percent |
| cds-synon | 5 | 1.916 |
| missense | 68 | 26.05 |
| UTR-3 | 3 | 1.149 |
| nearGene-3 | 9 | 3.448 |
| nearGene-5 | 4 | 1.533 |
| Intergenic | 99 | 37.93 |
| Intronic | 73 | 27.97 |
| Total | 261 | 100 |

1.2 Identification of Small TransRNAs Encoded by Intergenic Sequences Containing Disease-Linked SNPs

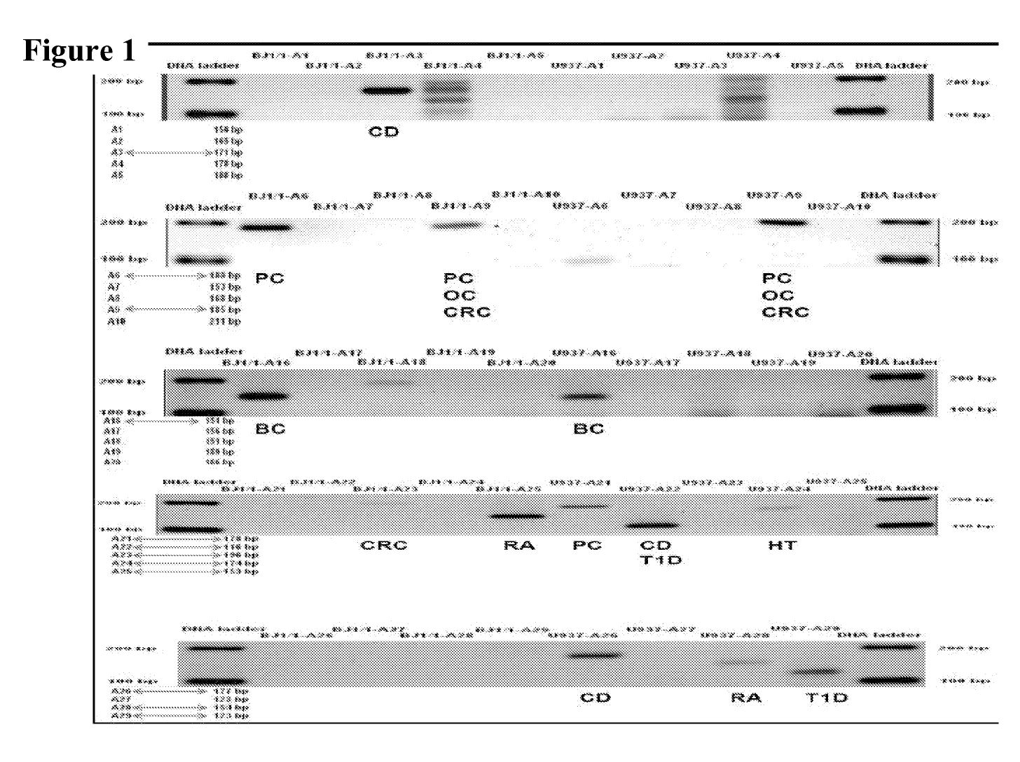

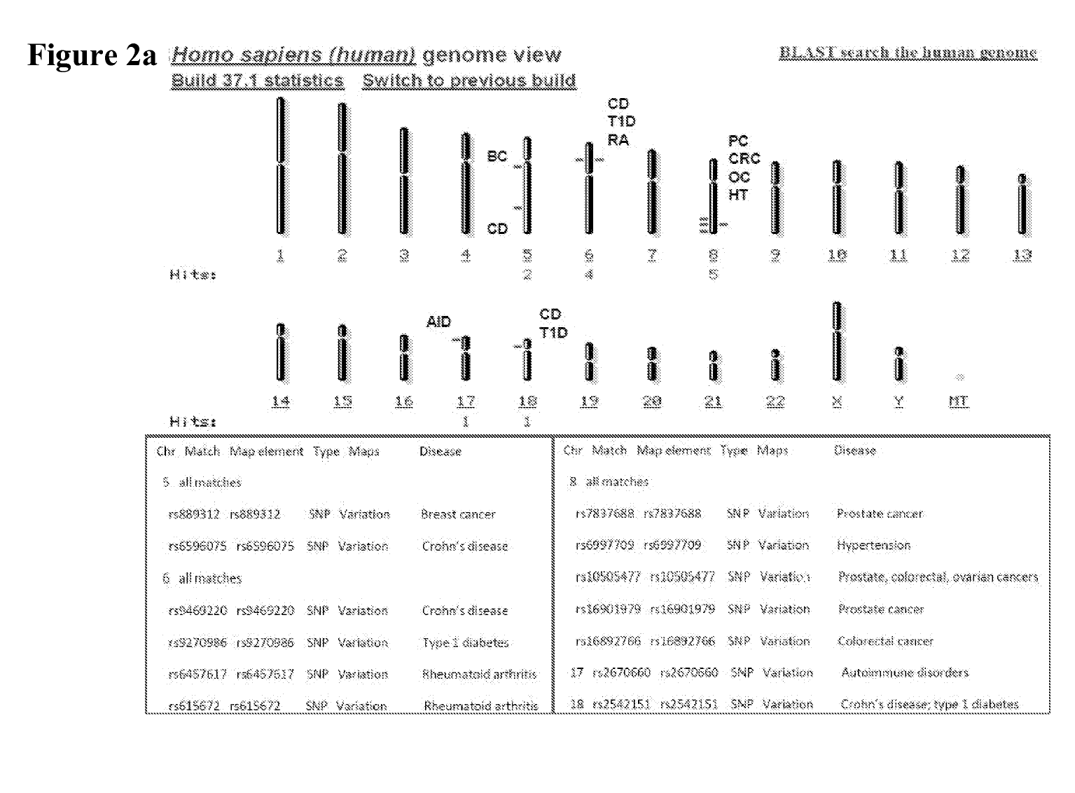

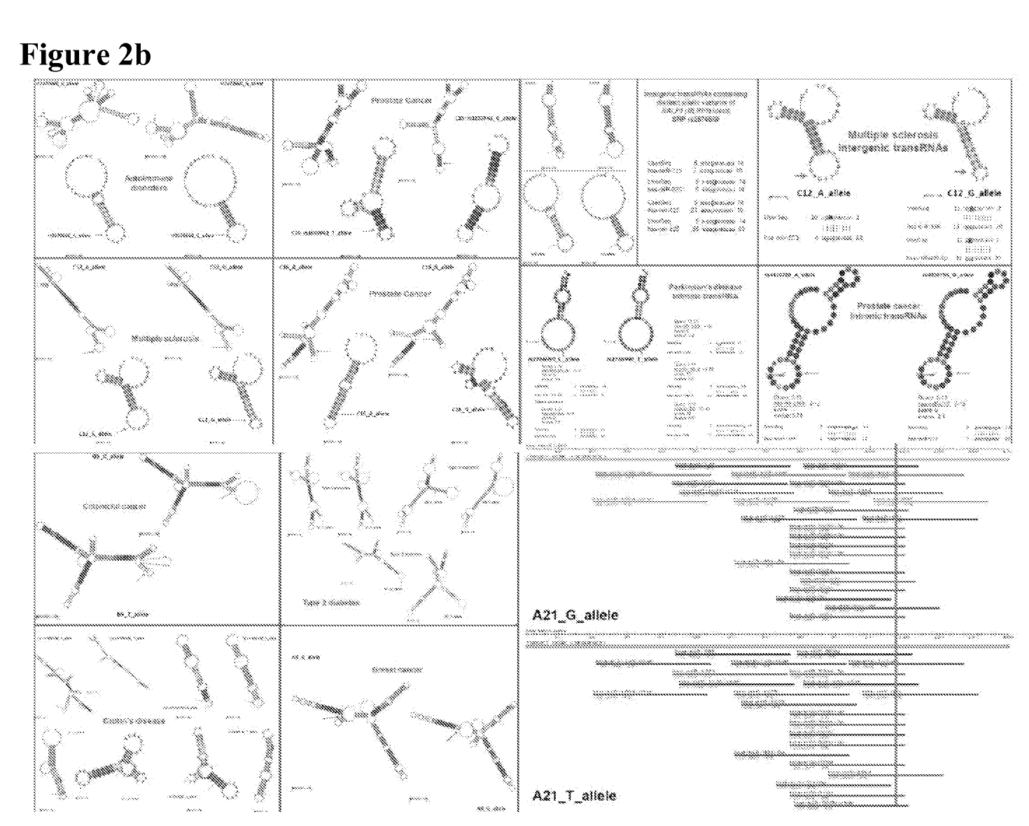

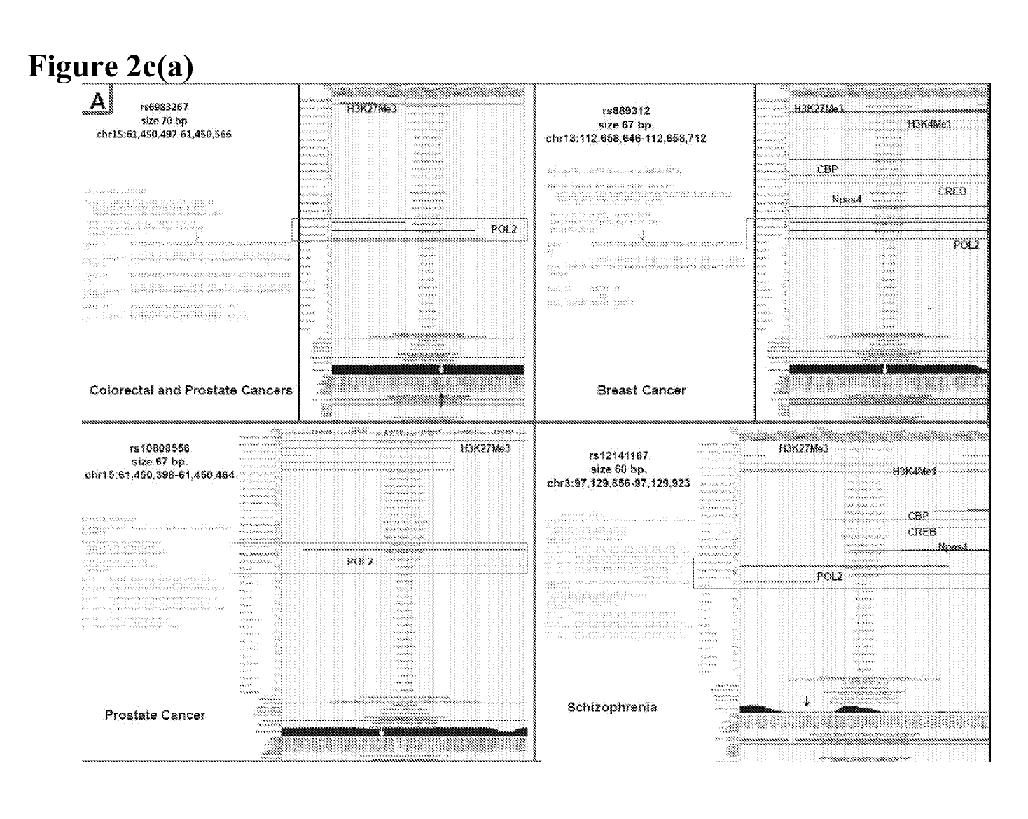

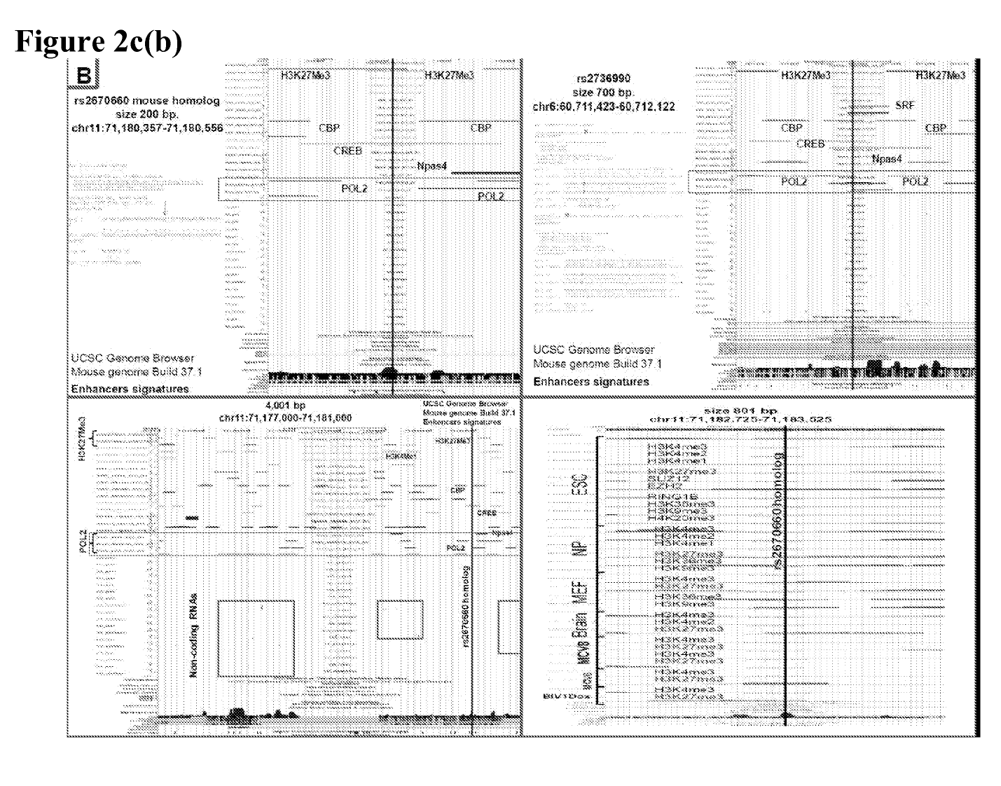

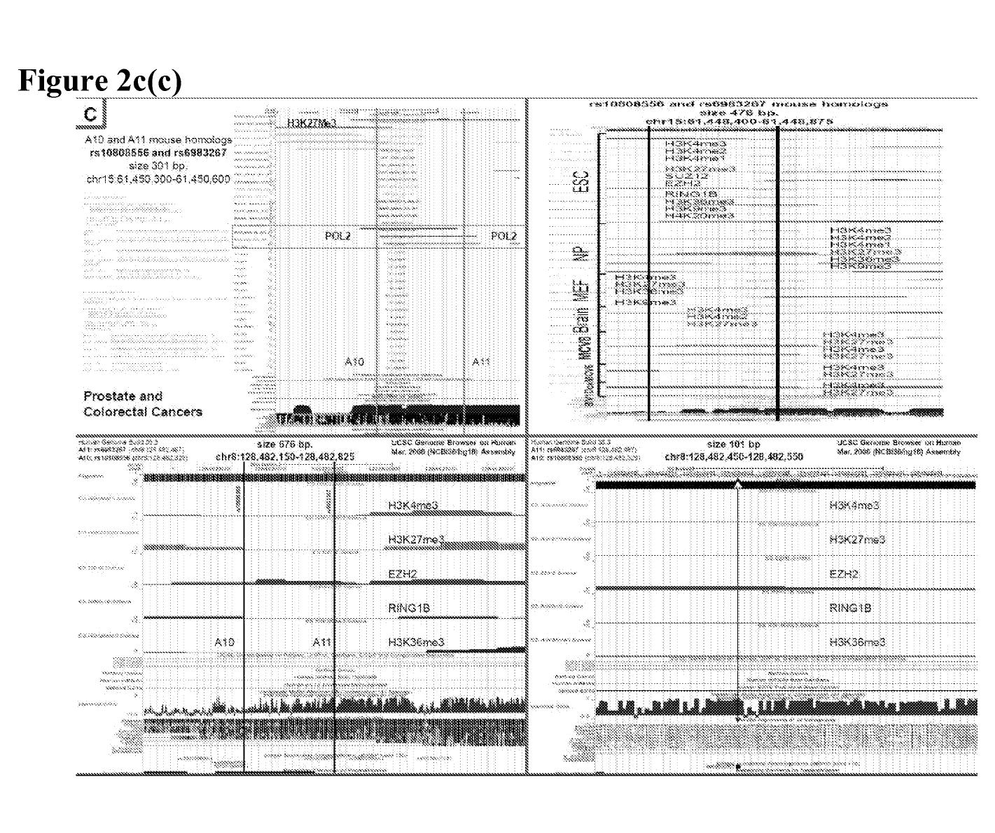

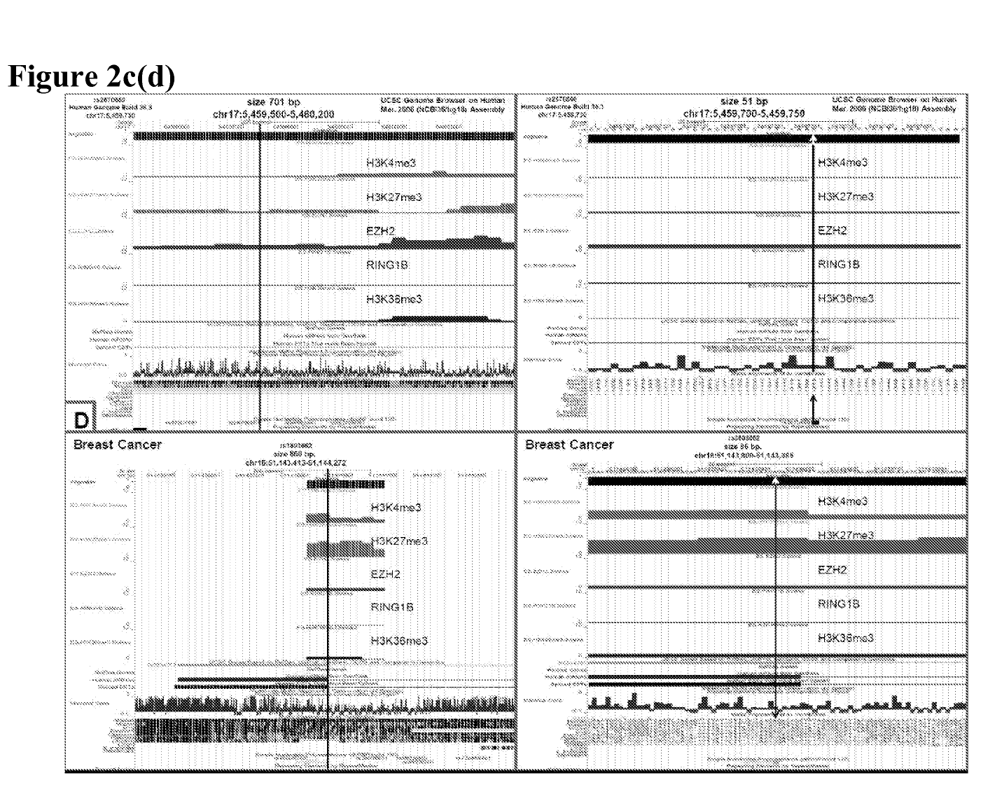

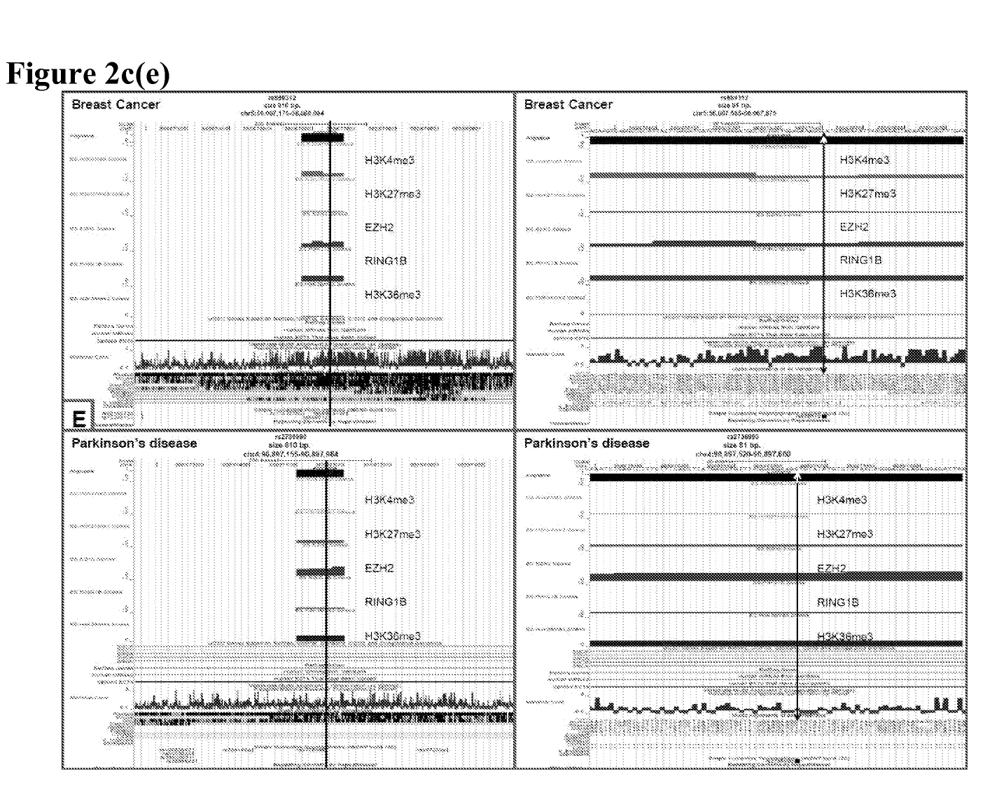

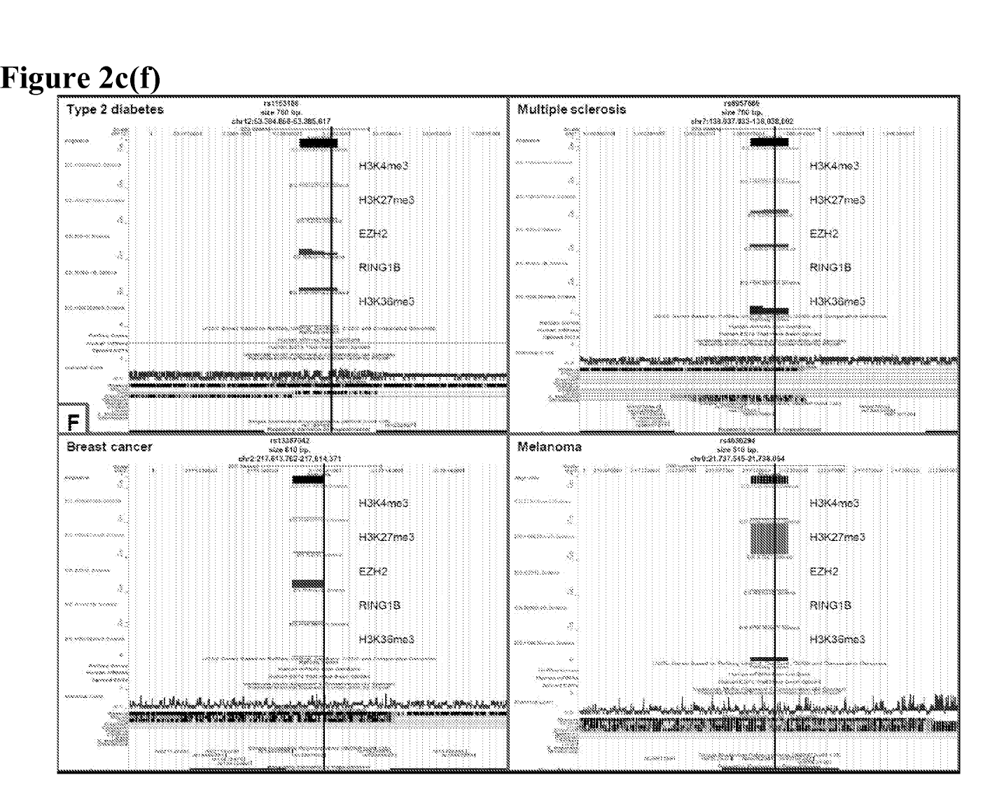

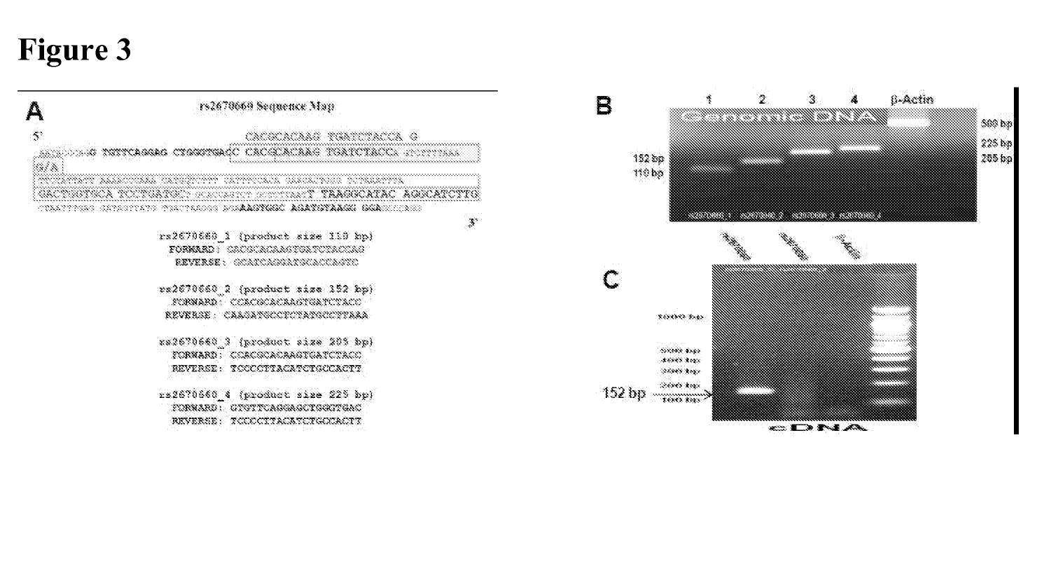

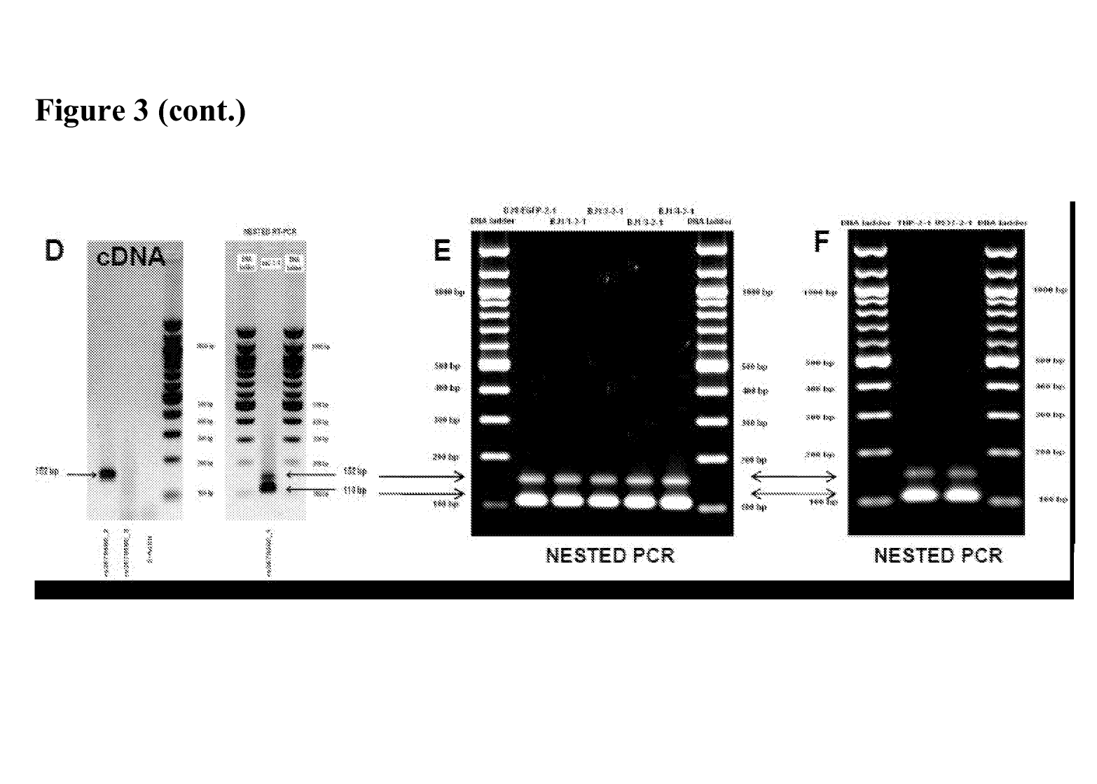

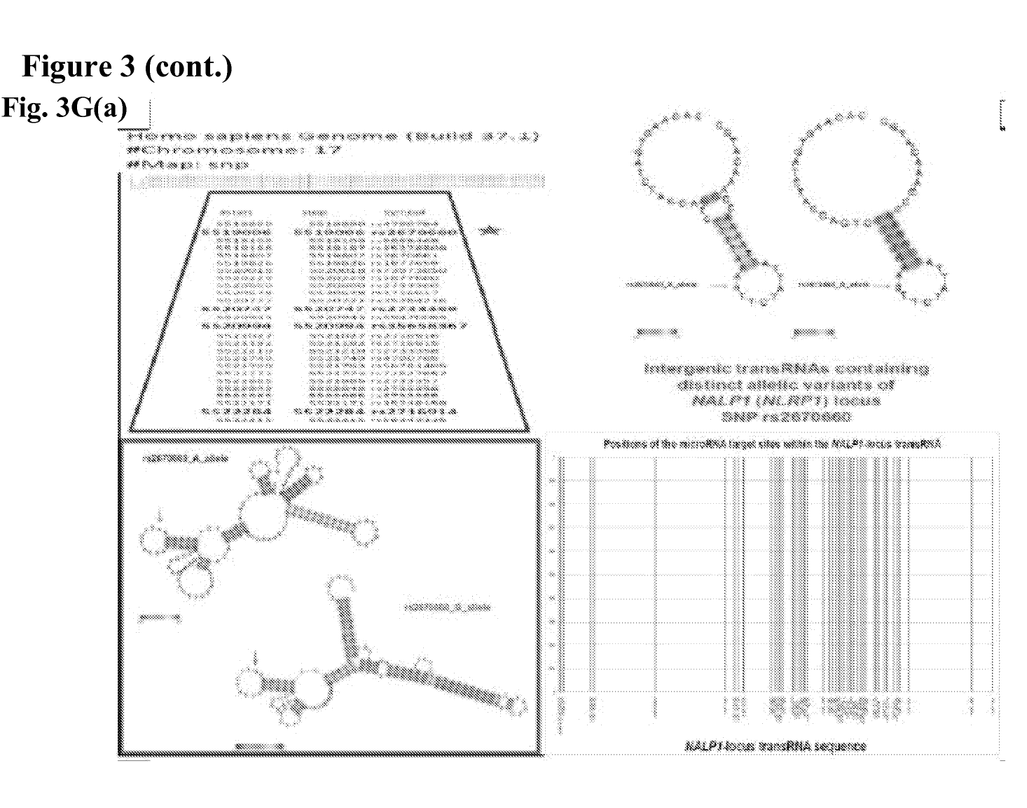

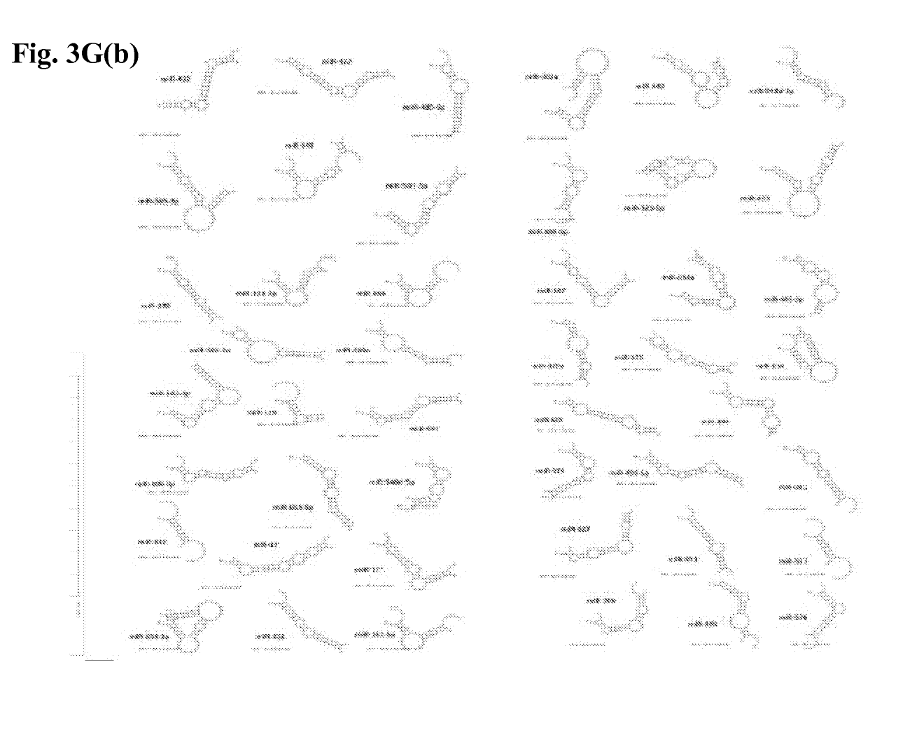

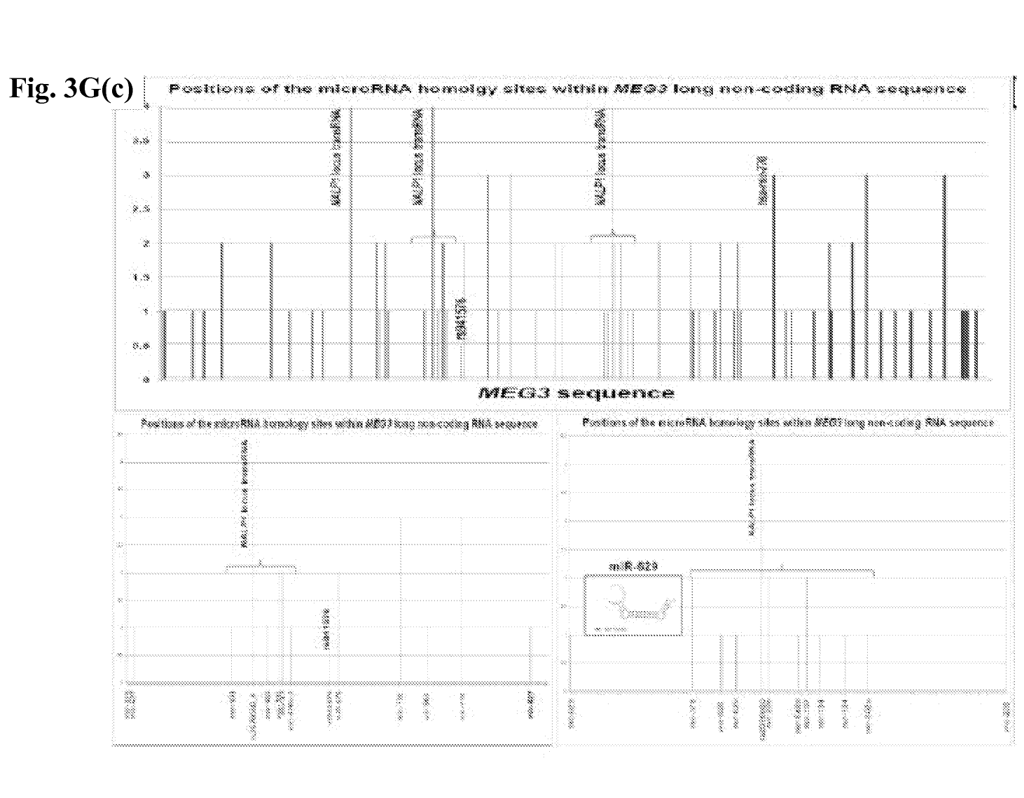

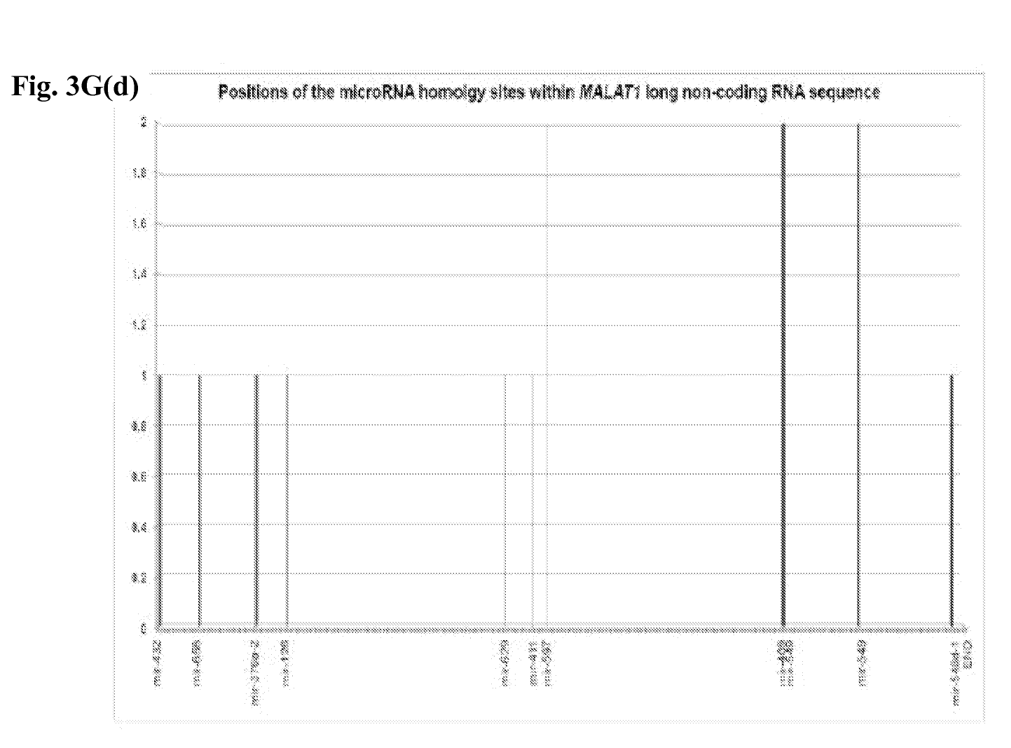

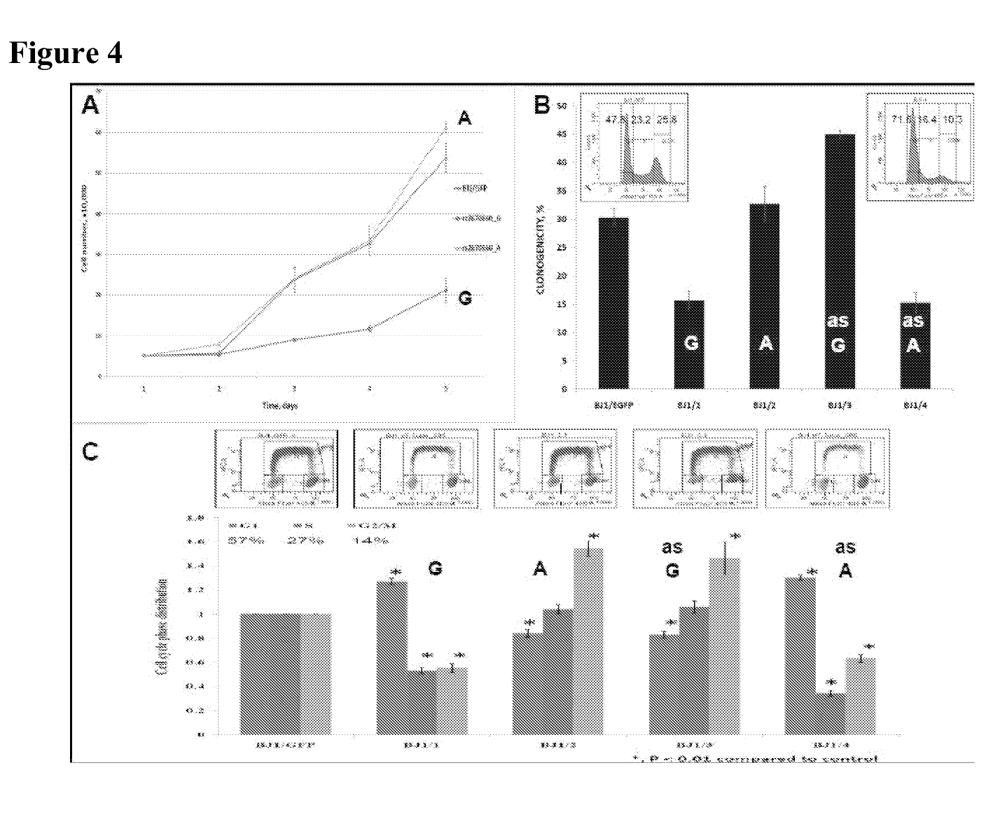

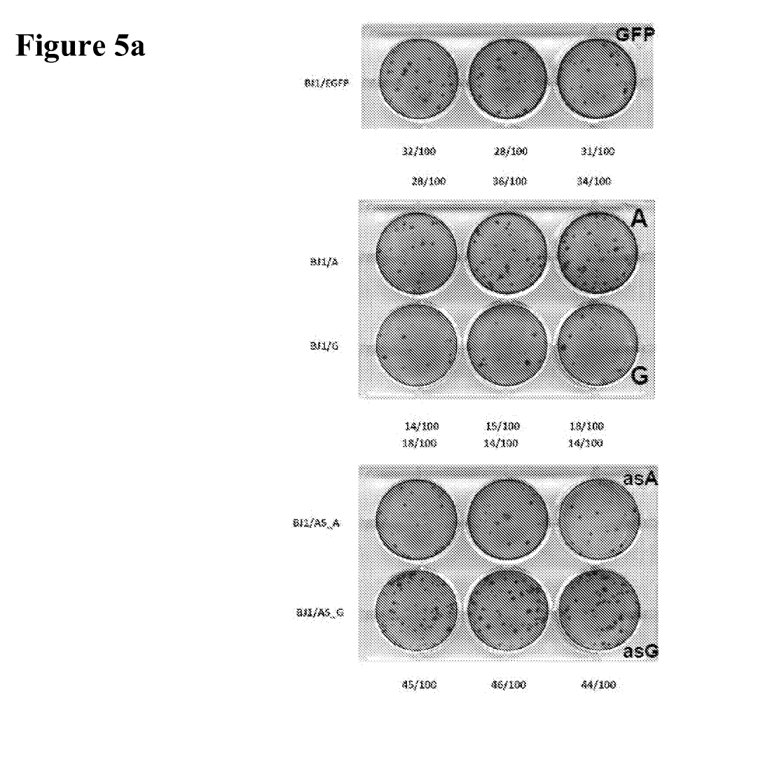

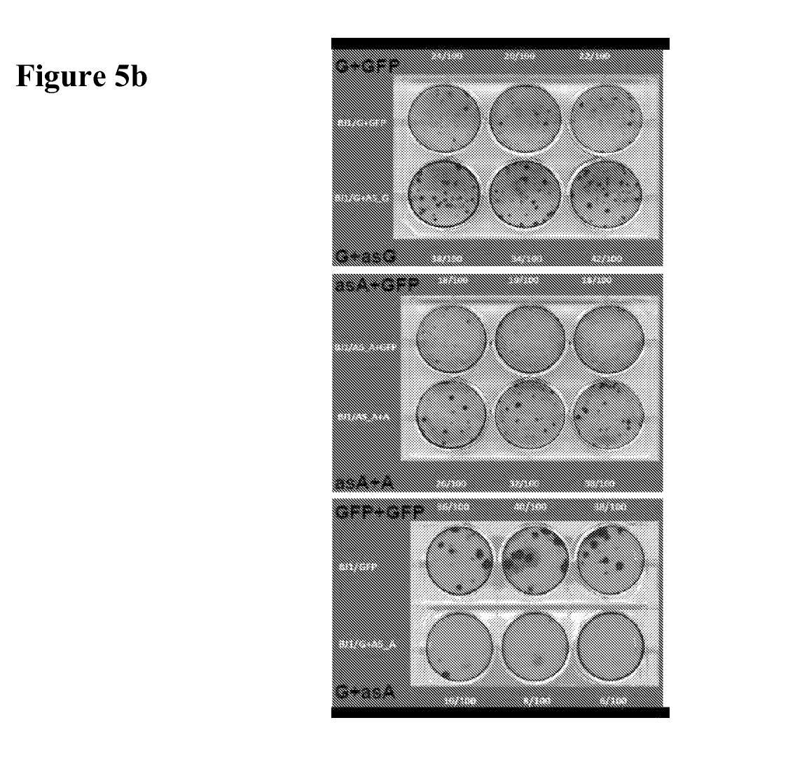

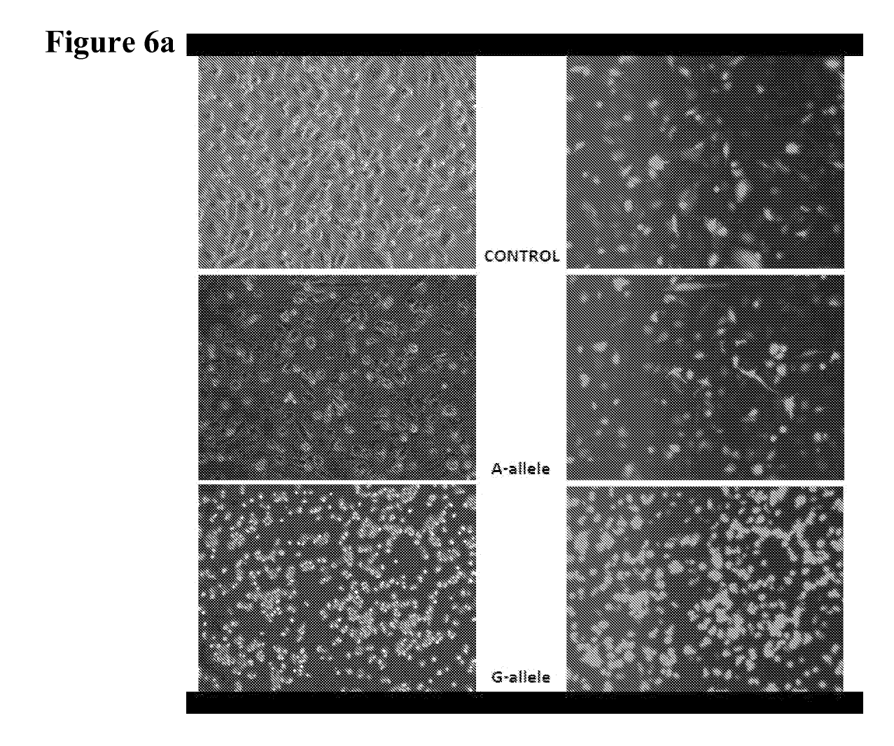

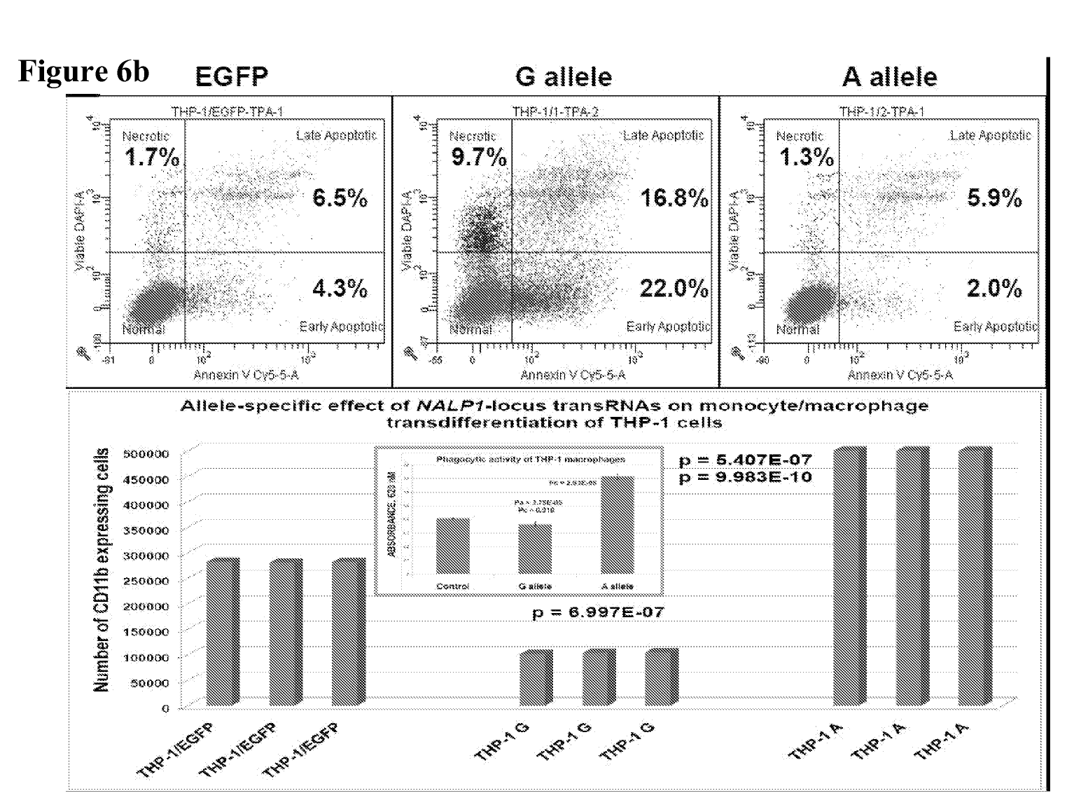

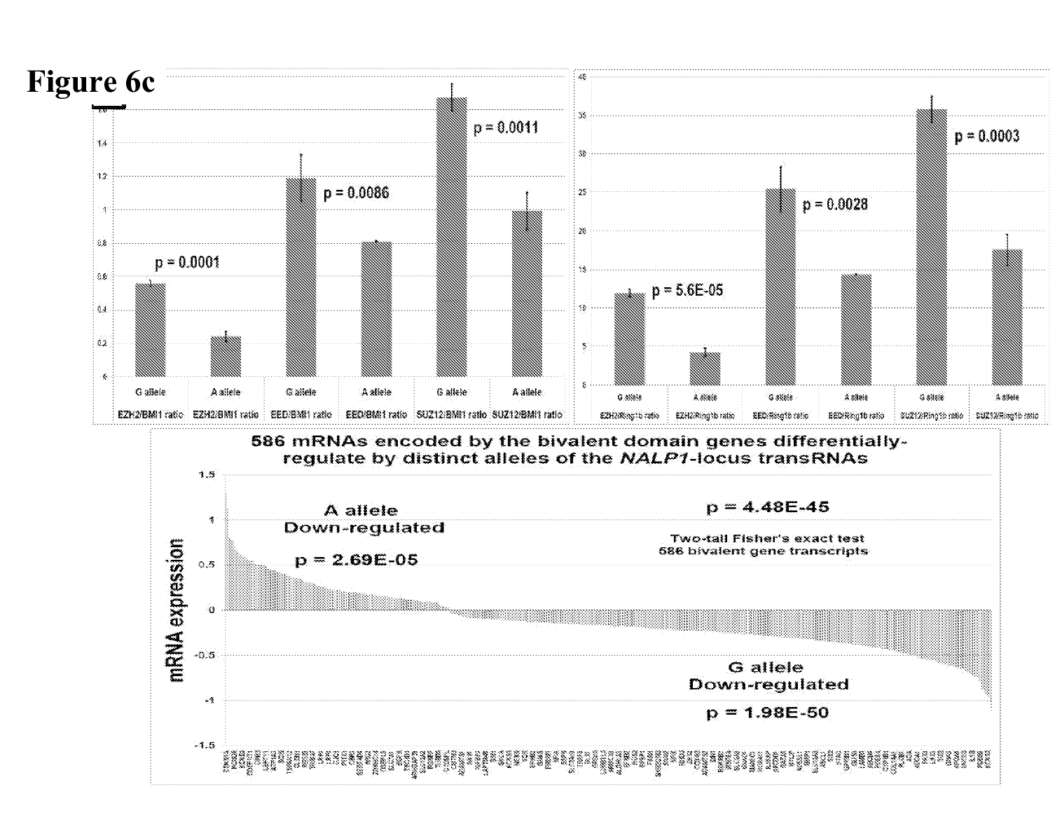

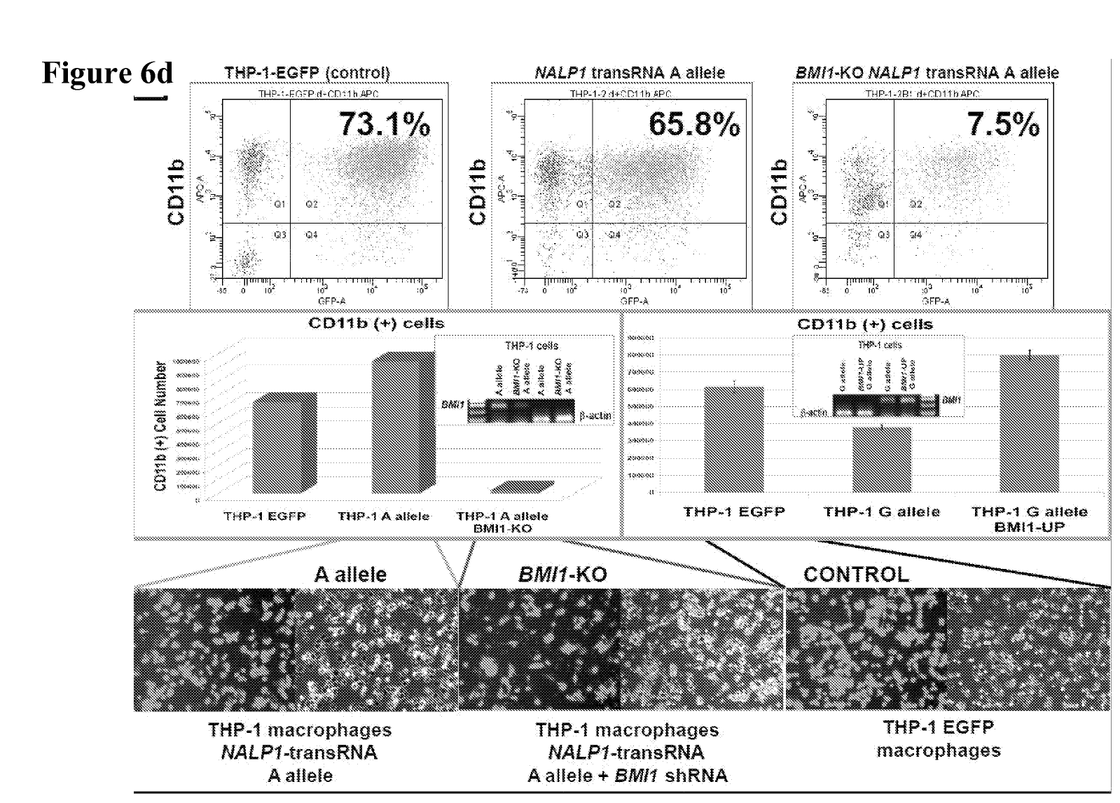

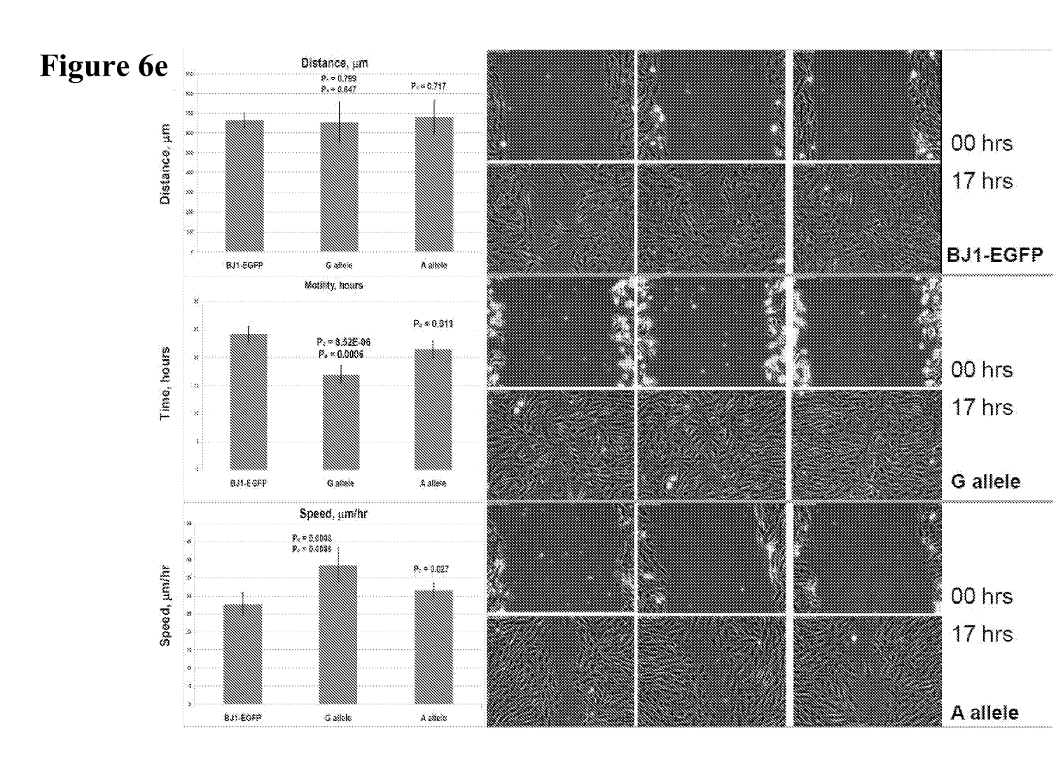

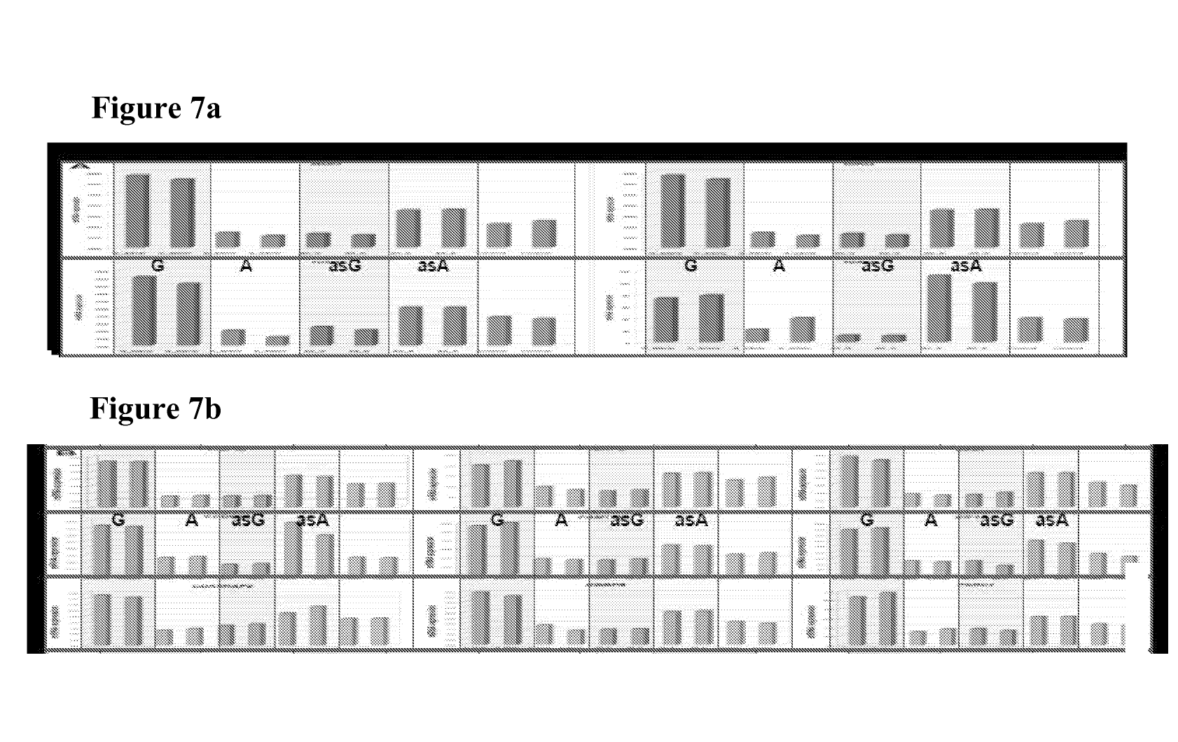

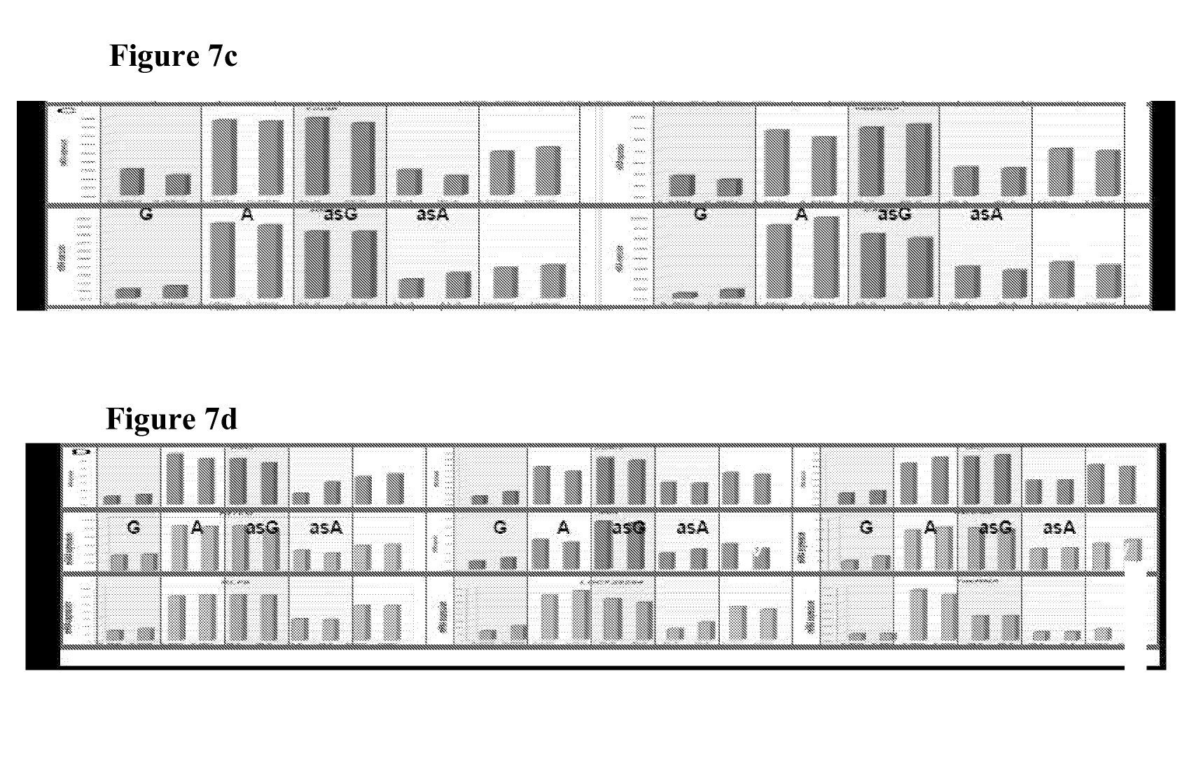

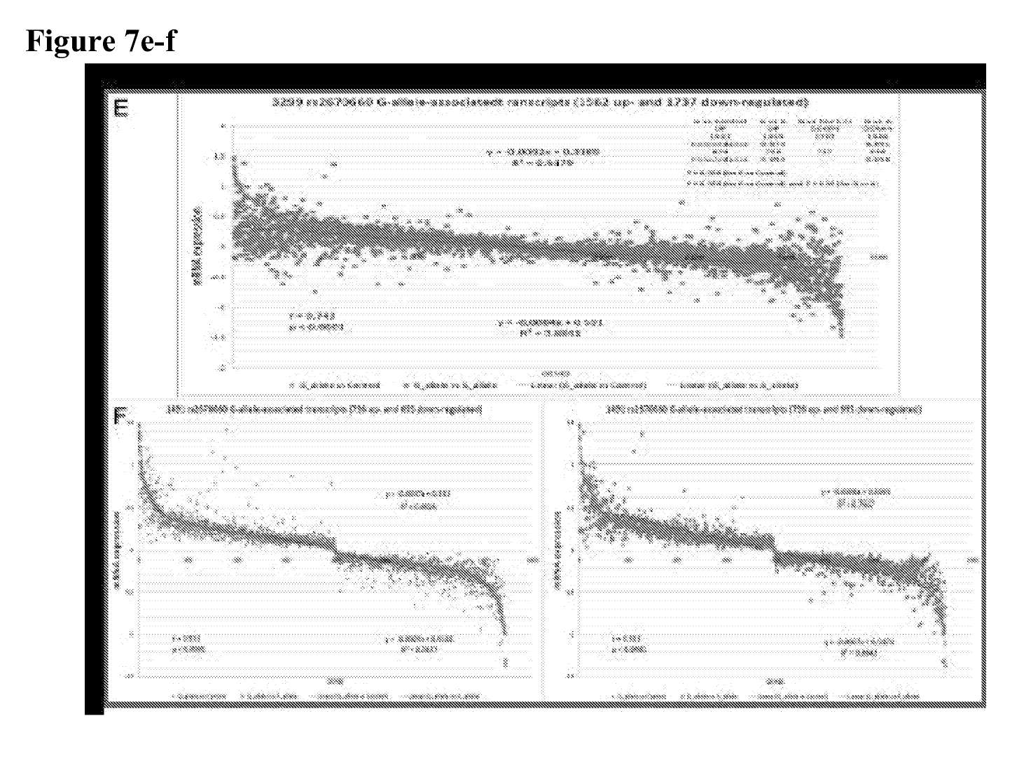

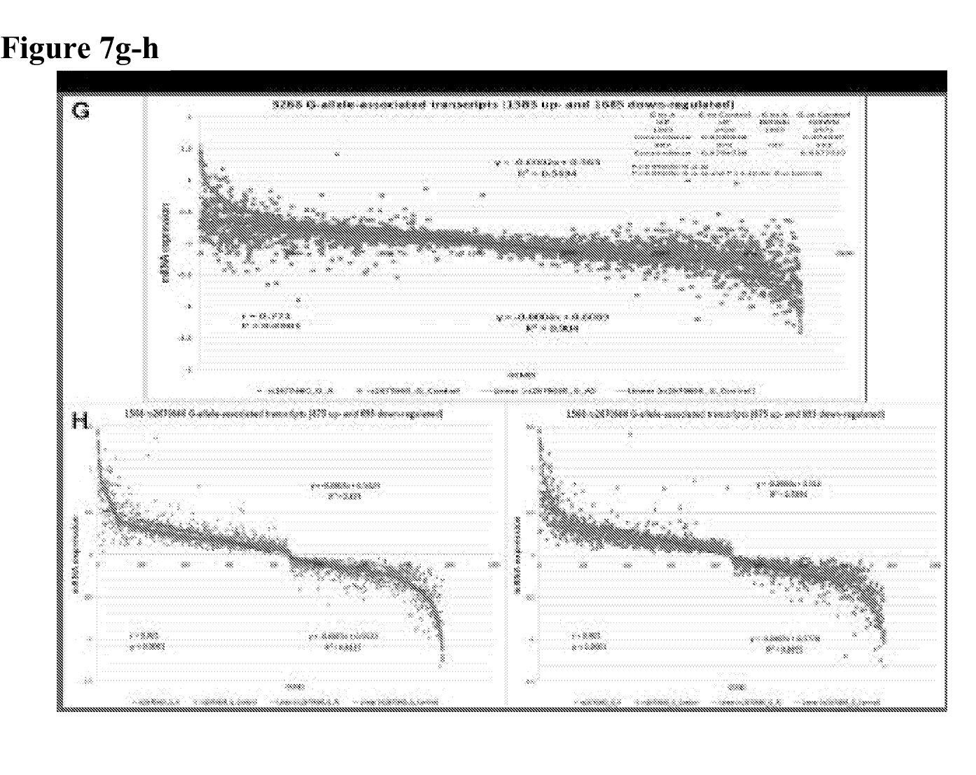

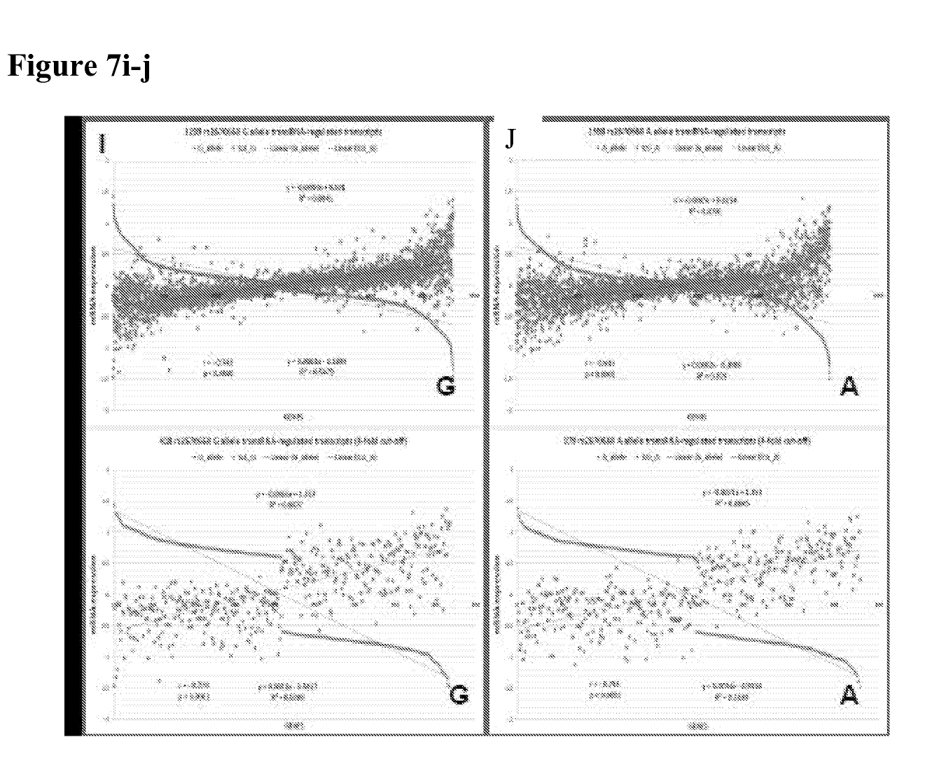

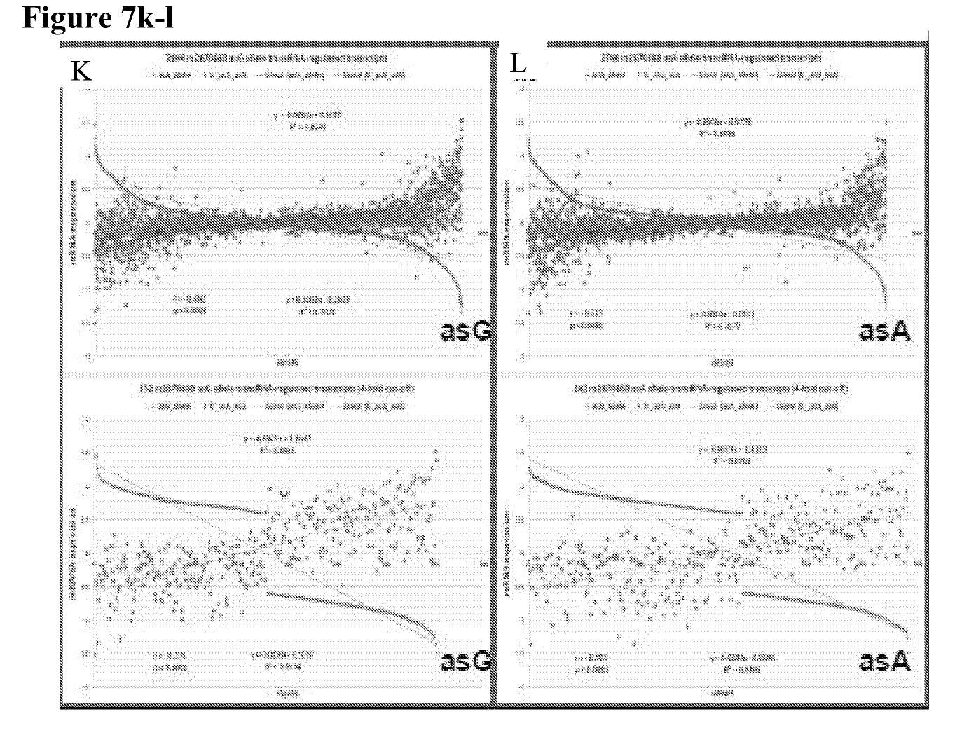

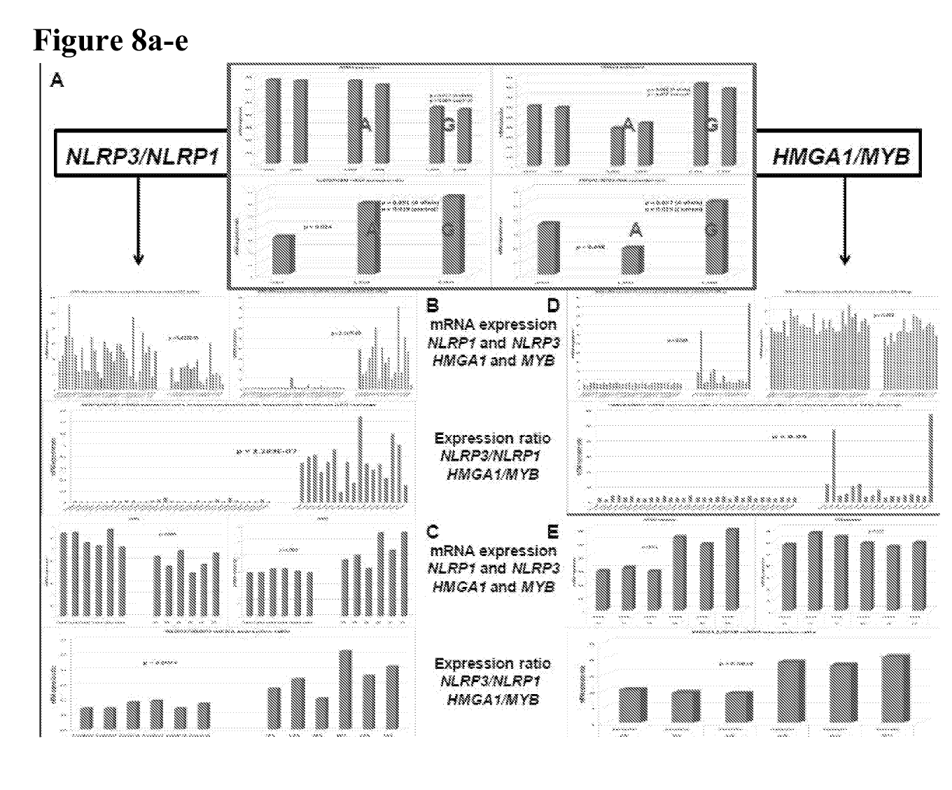

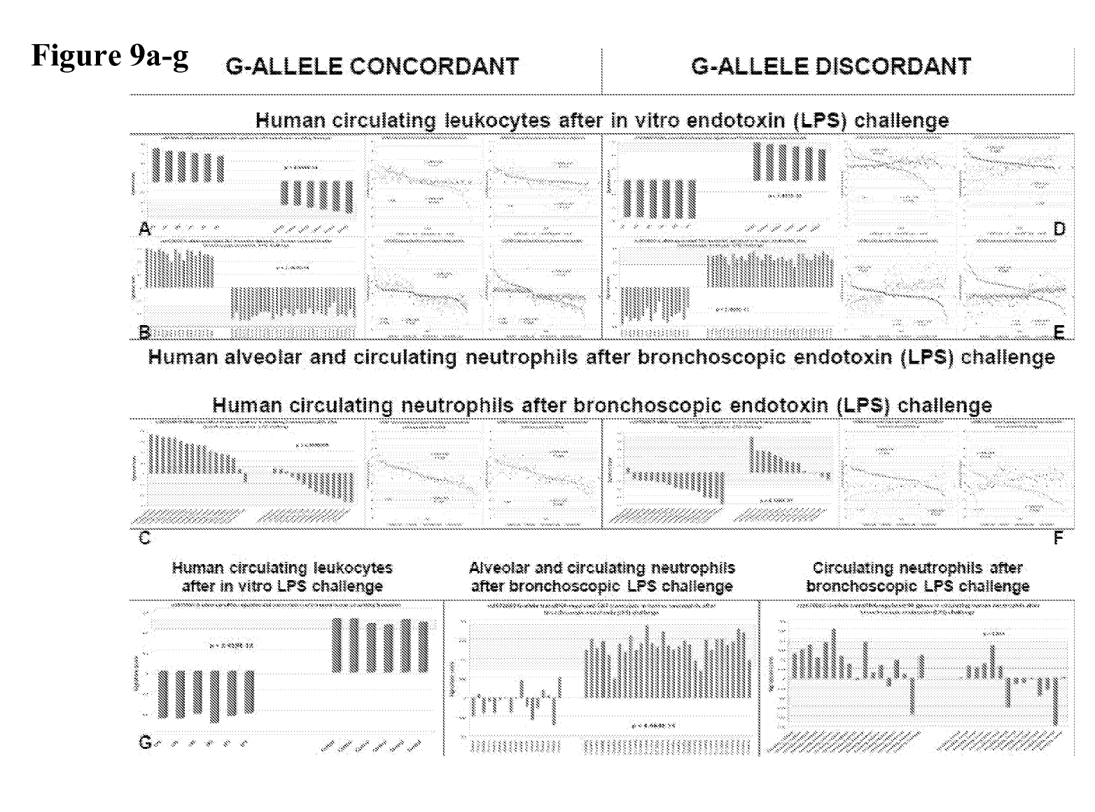

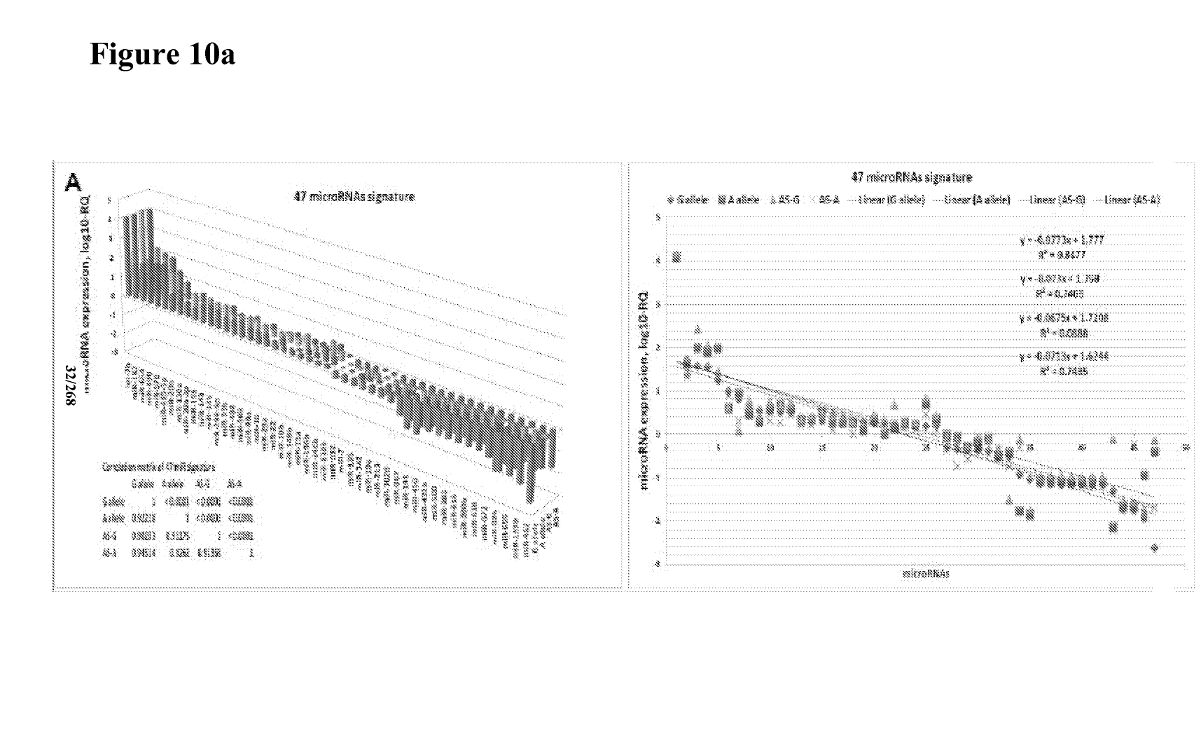

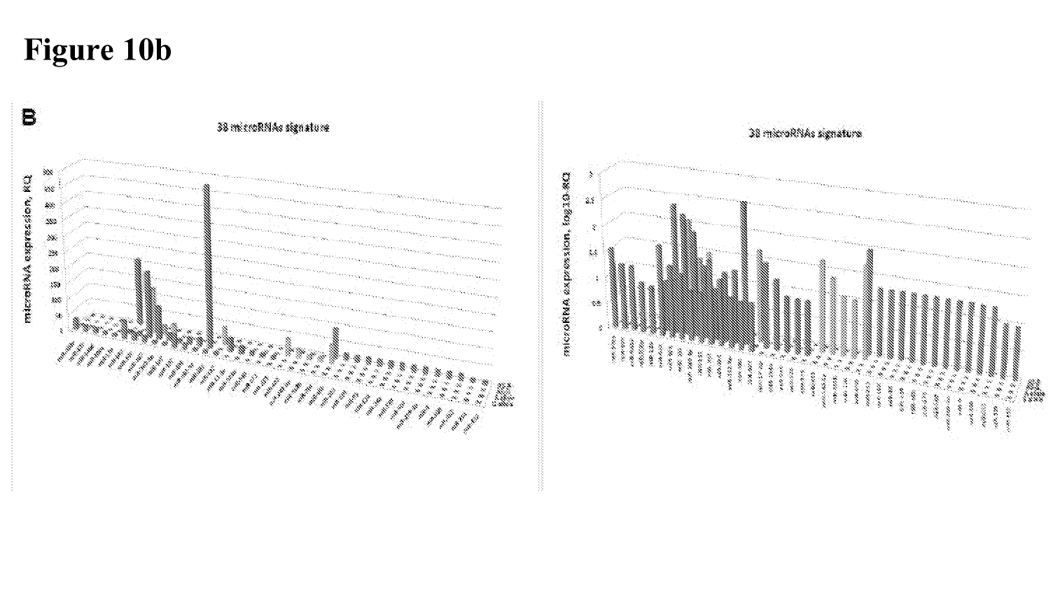

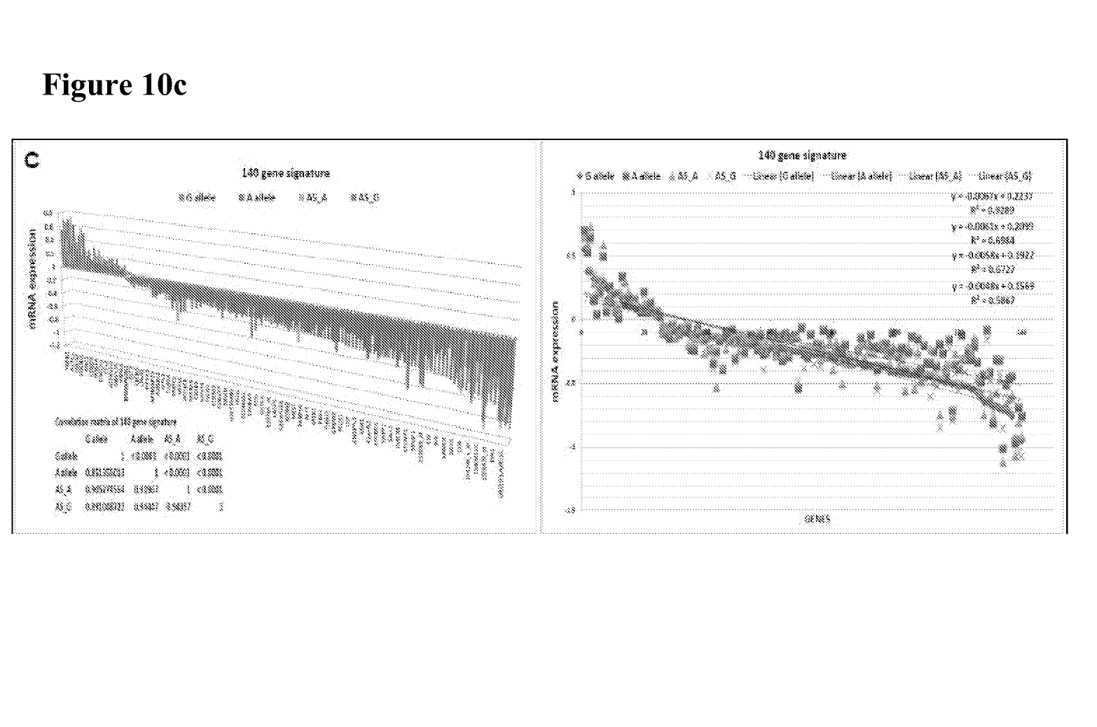

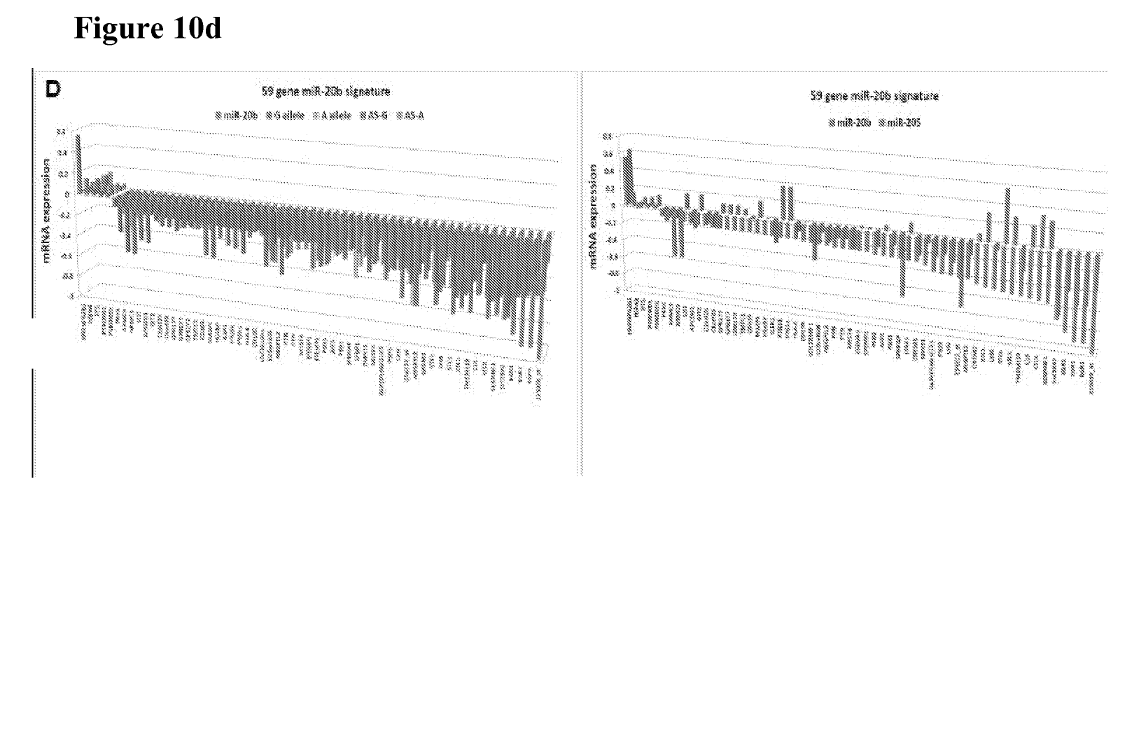

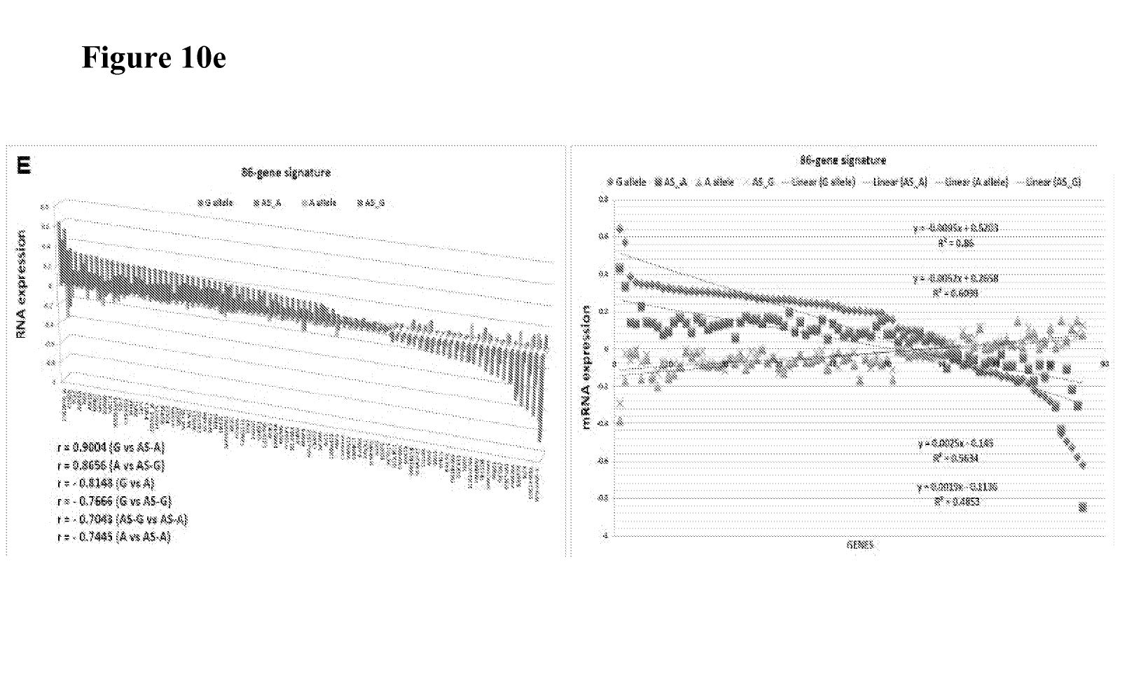

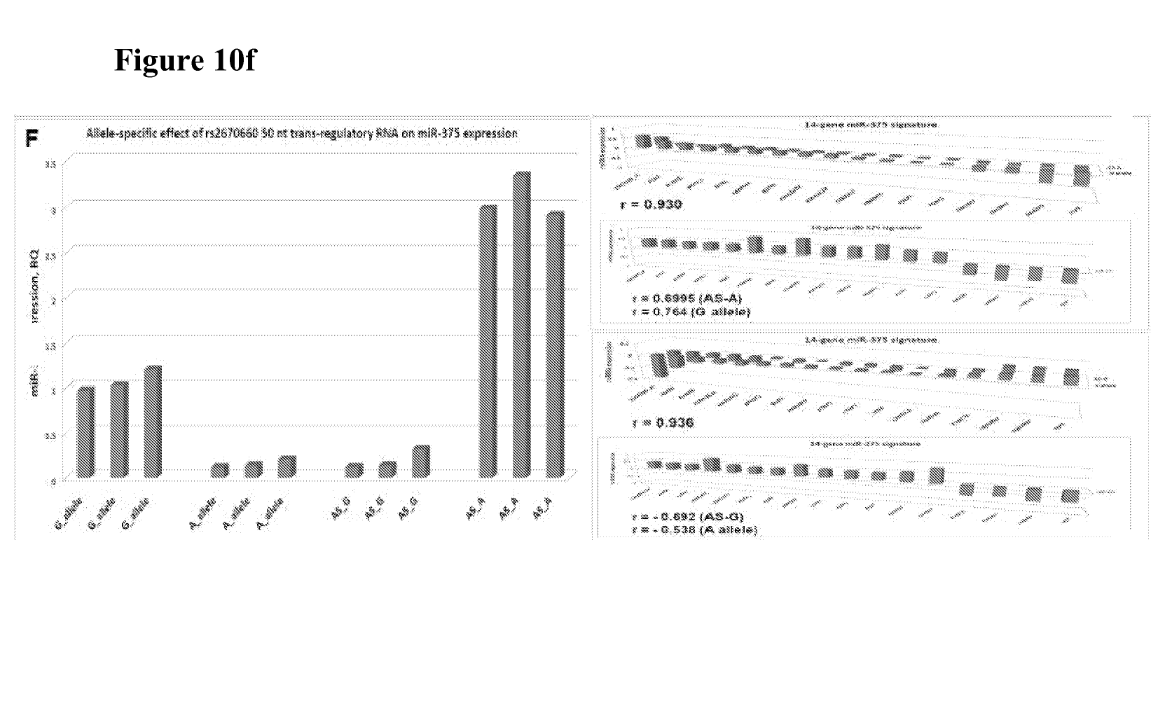

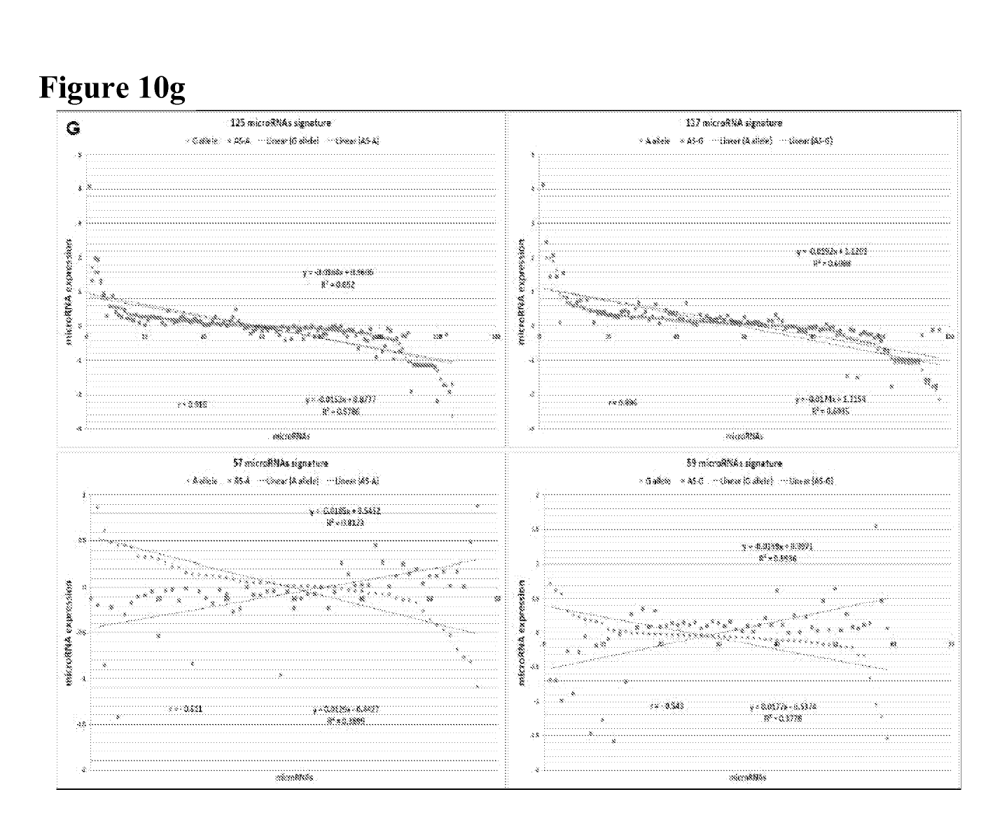

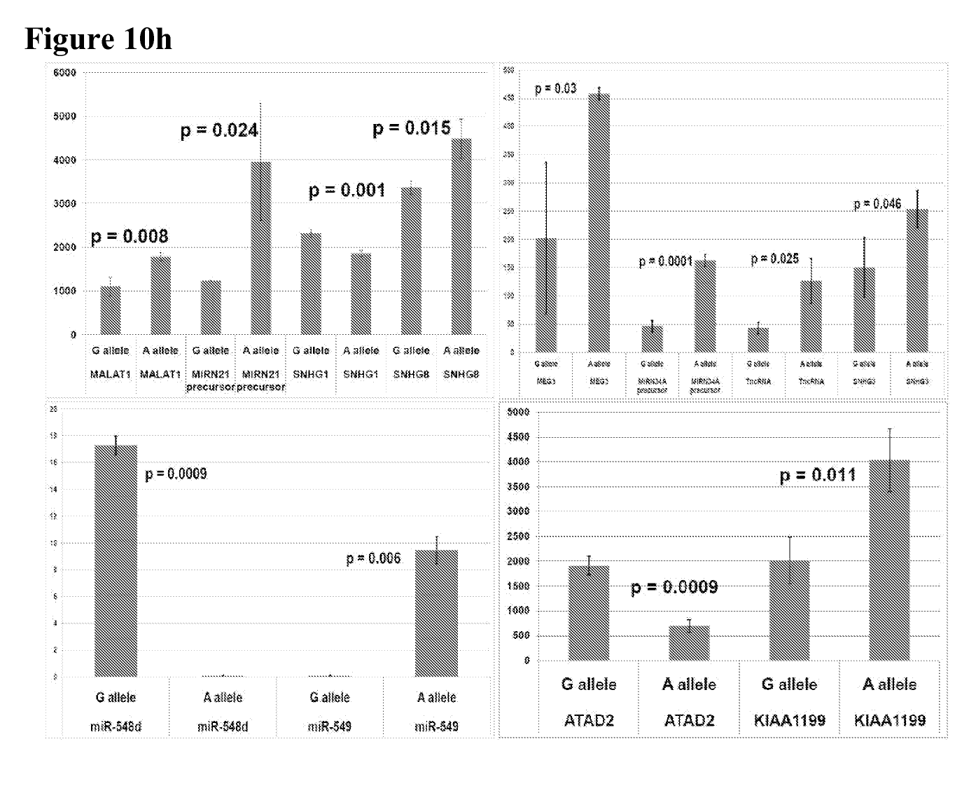

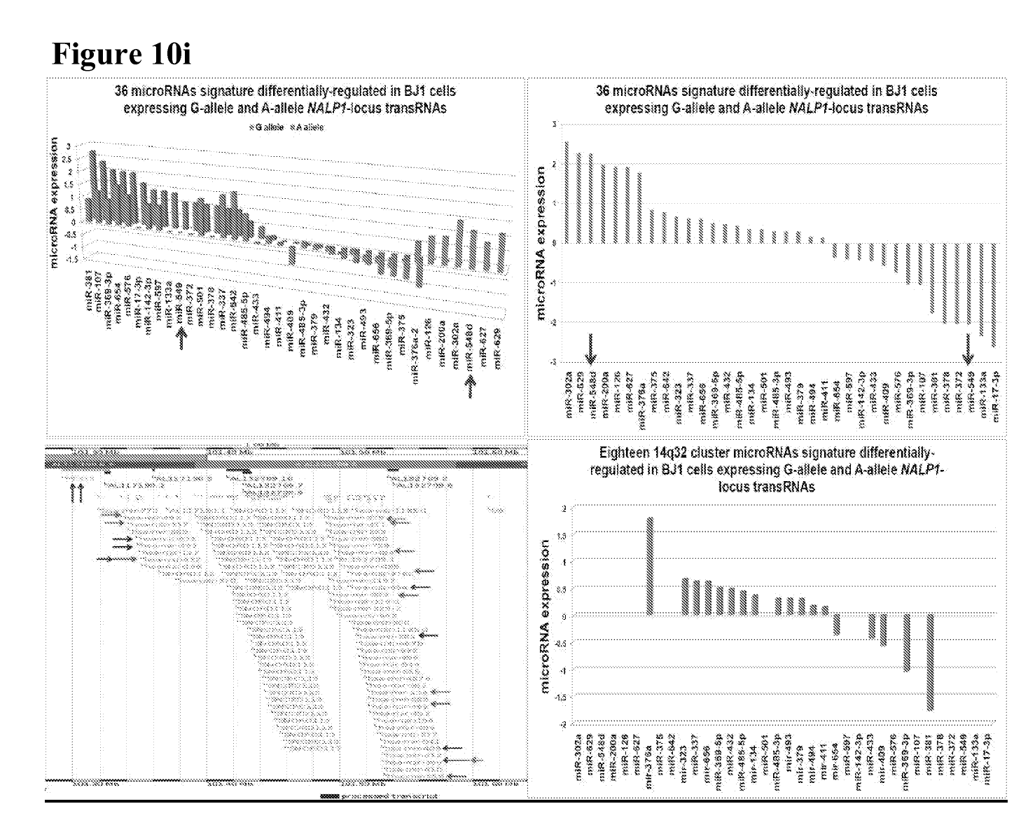

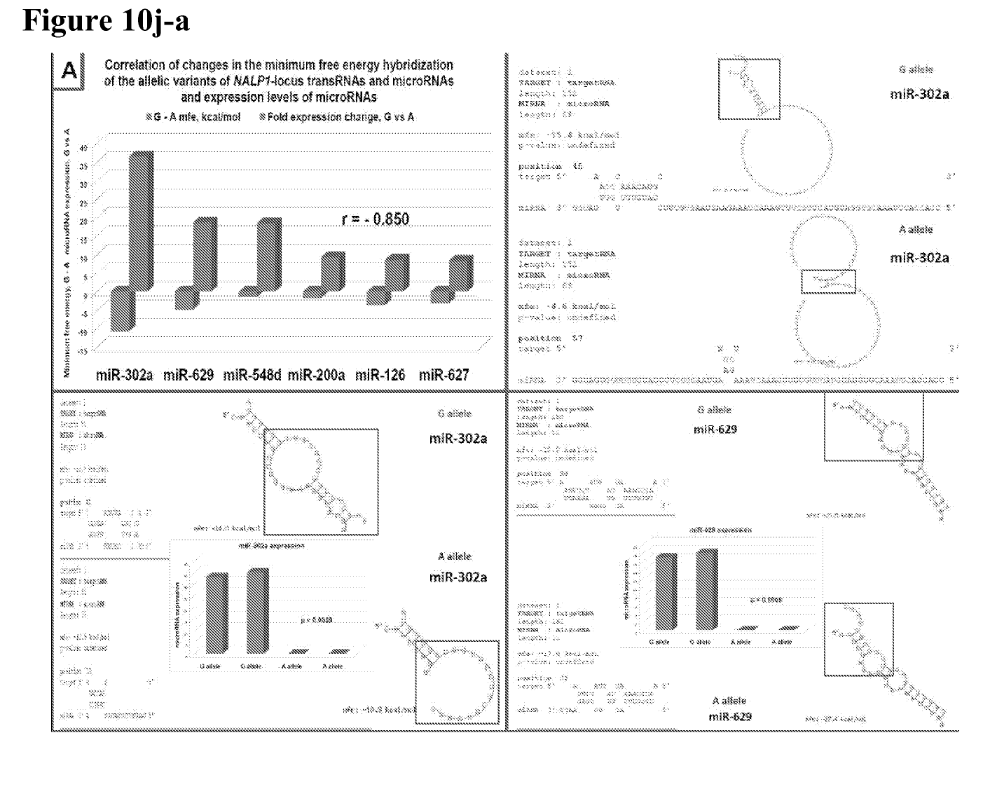

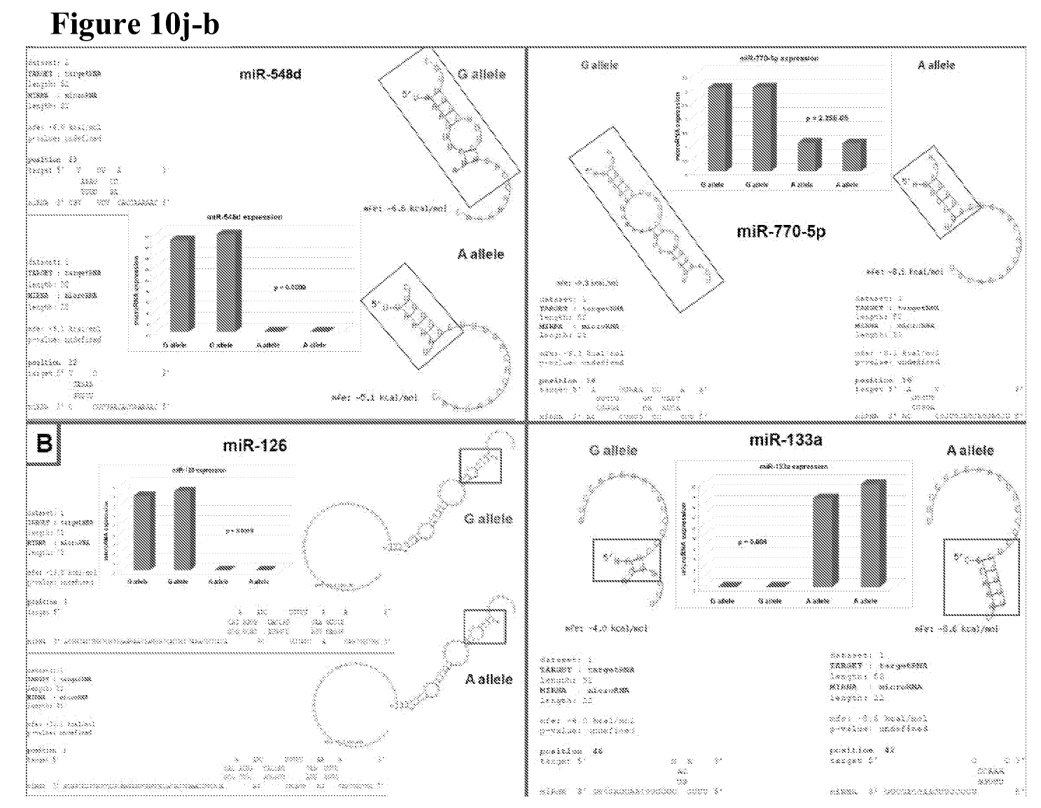

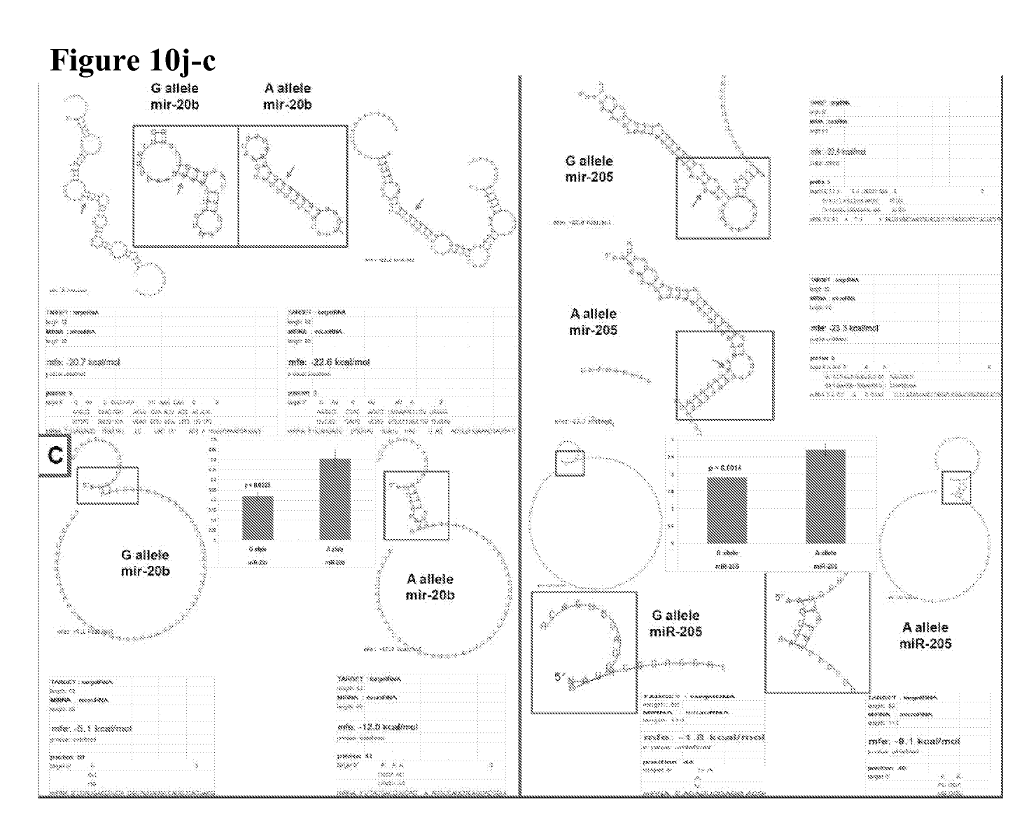

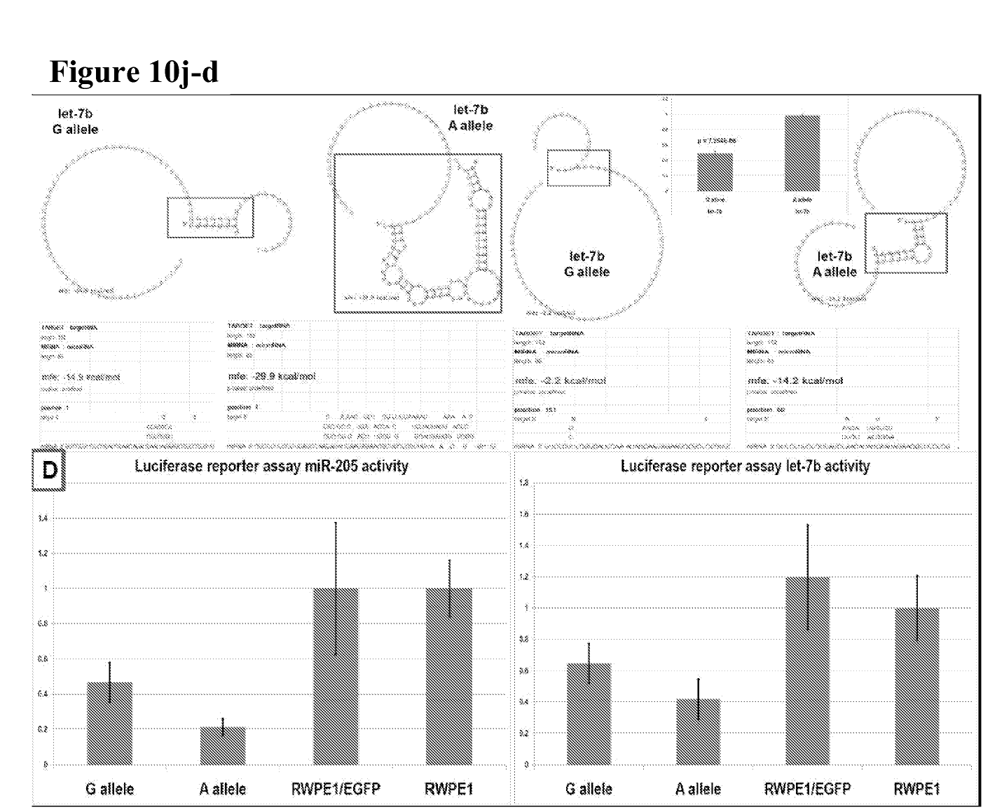

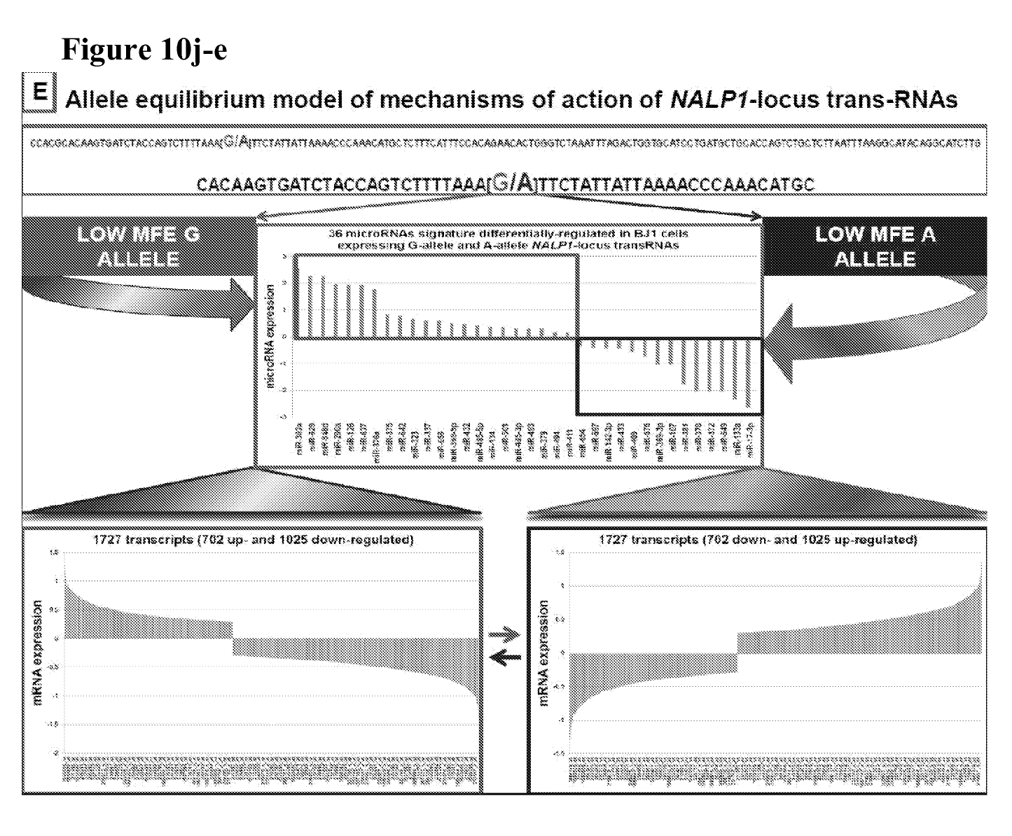

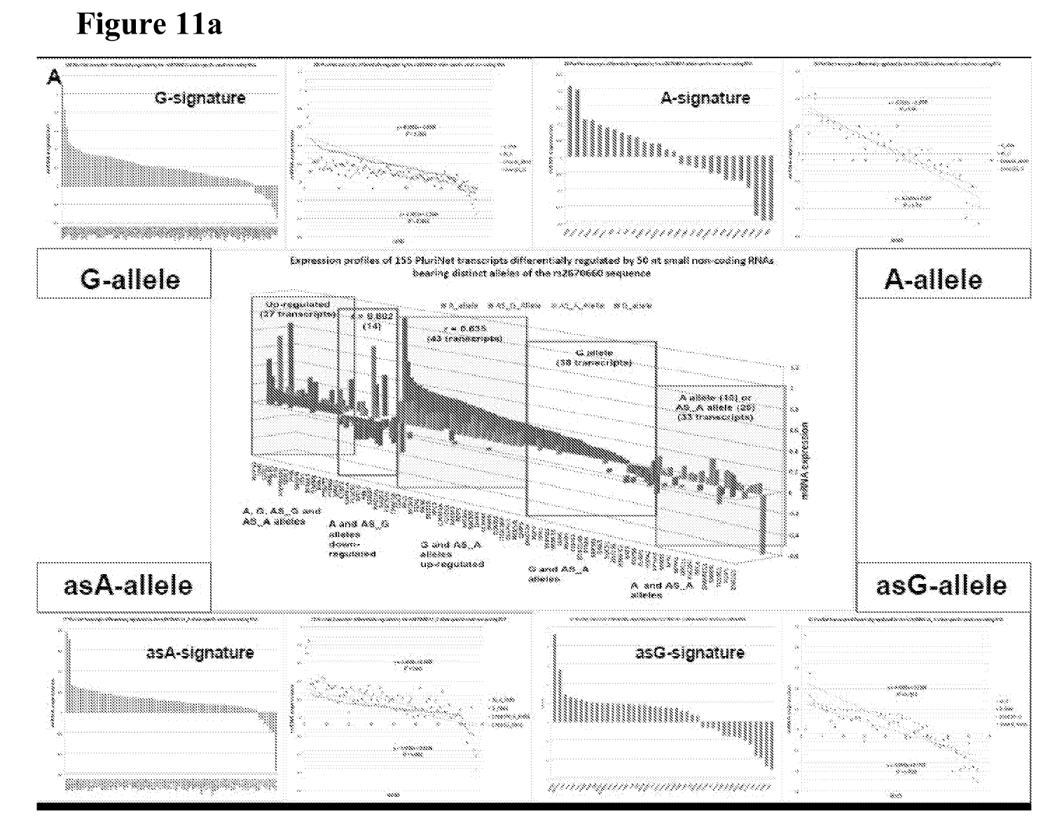

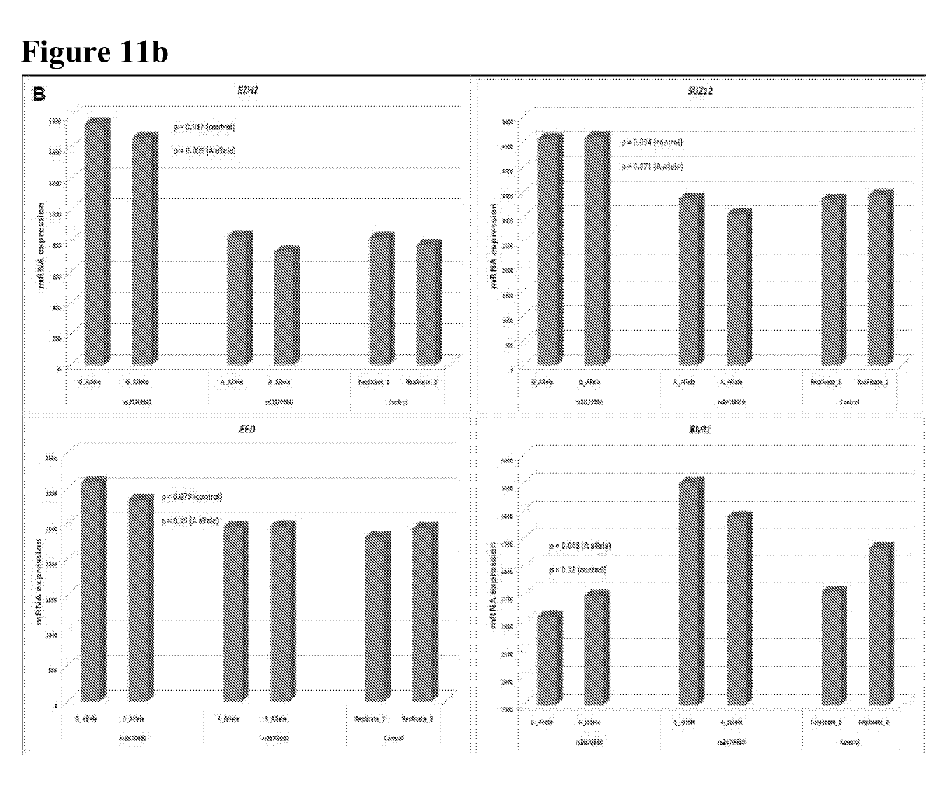

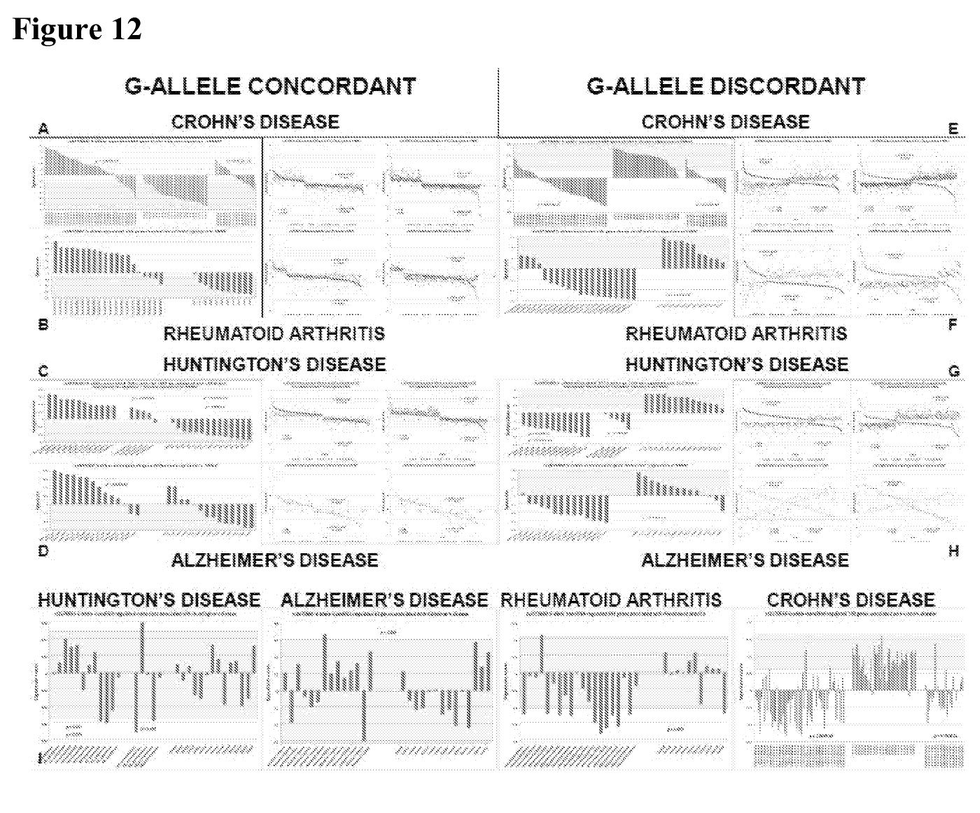

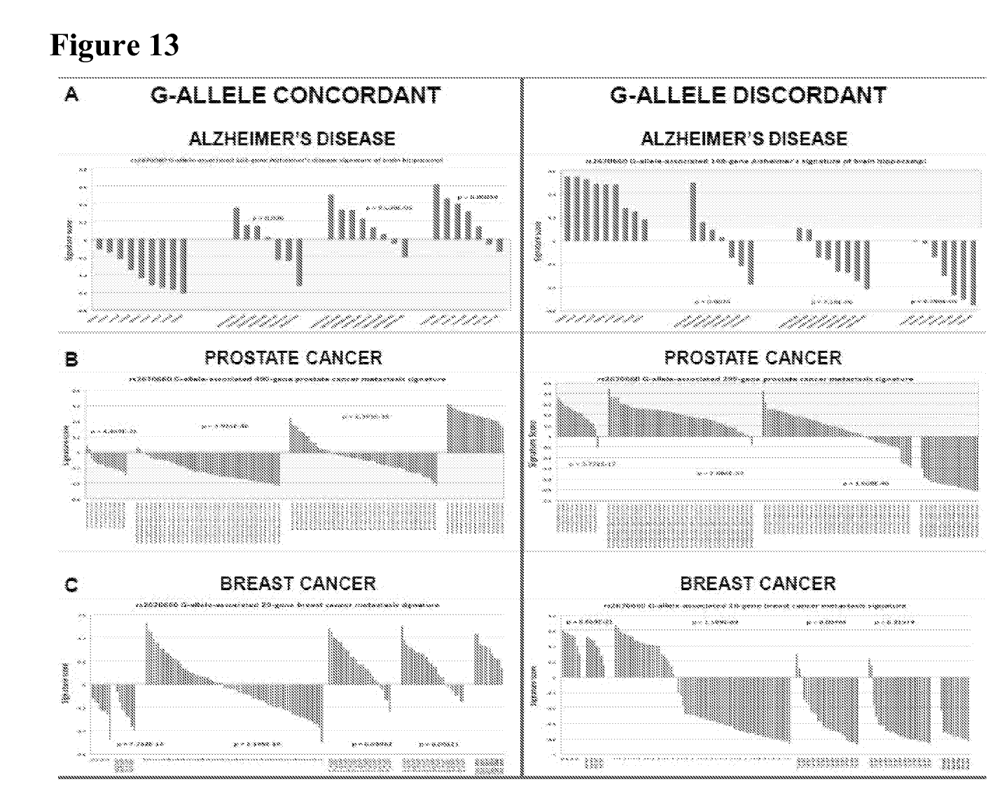

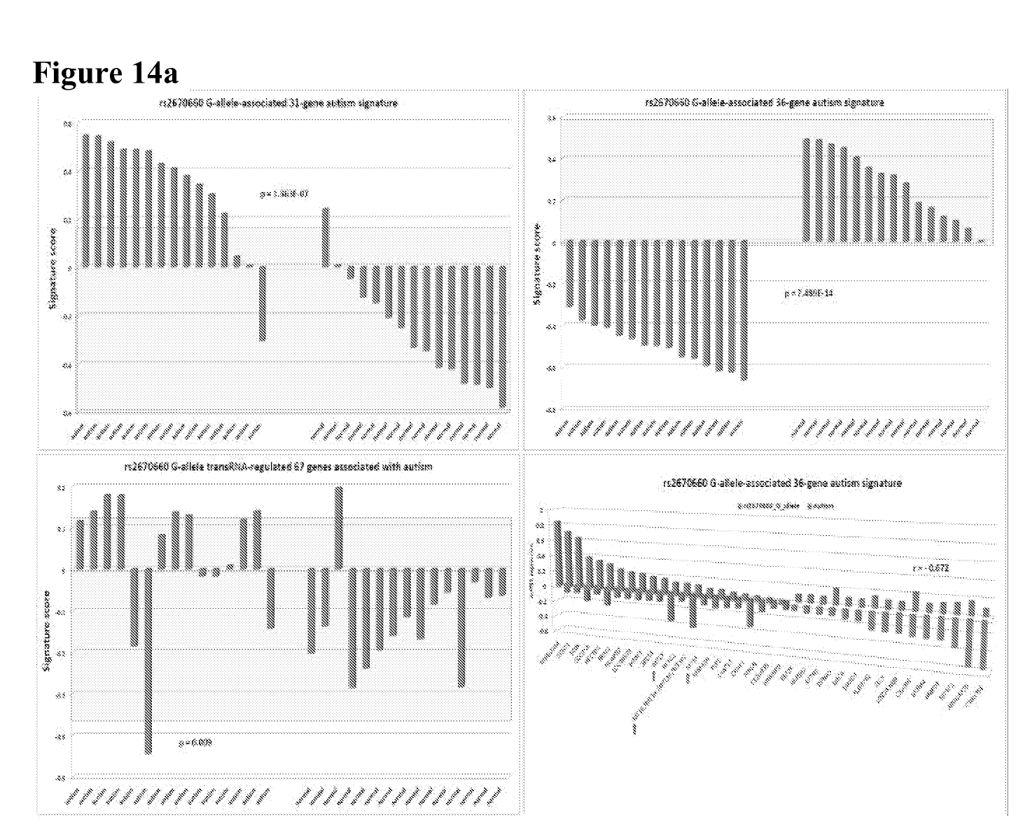

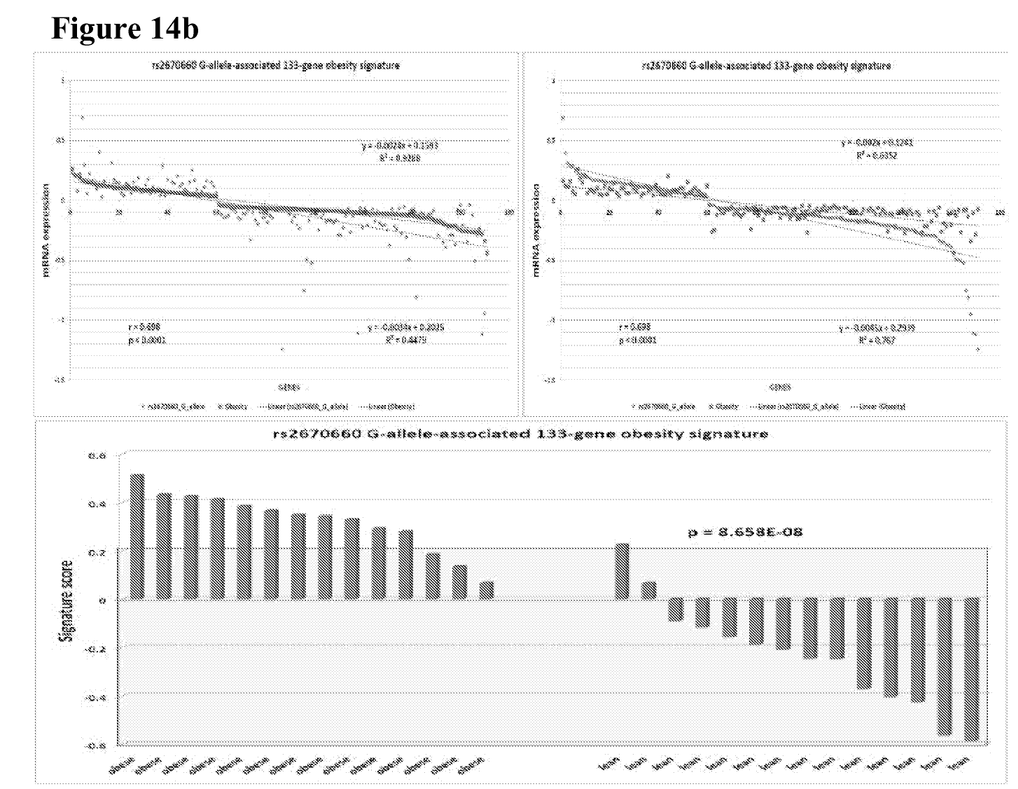

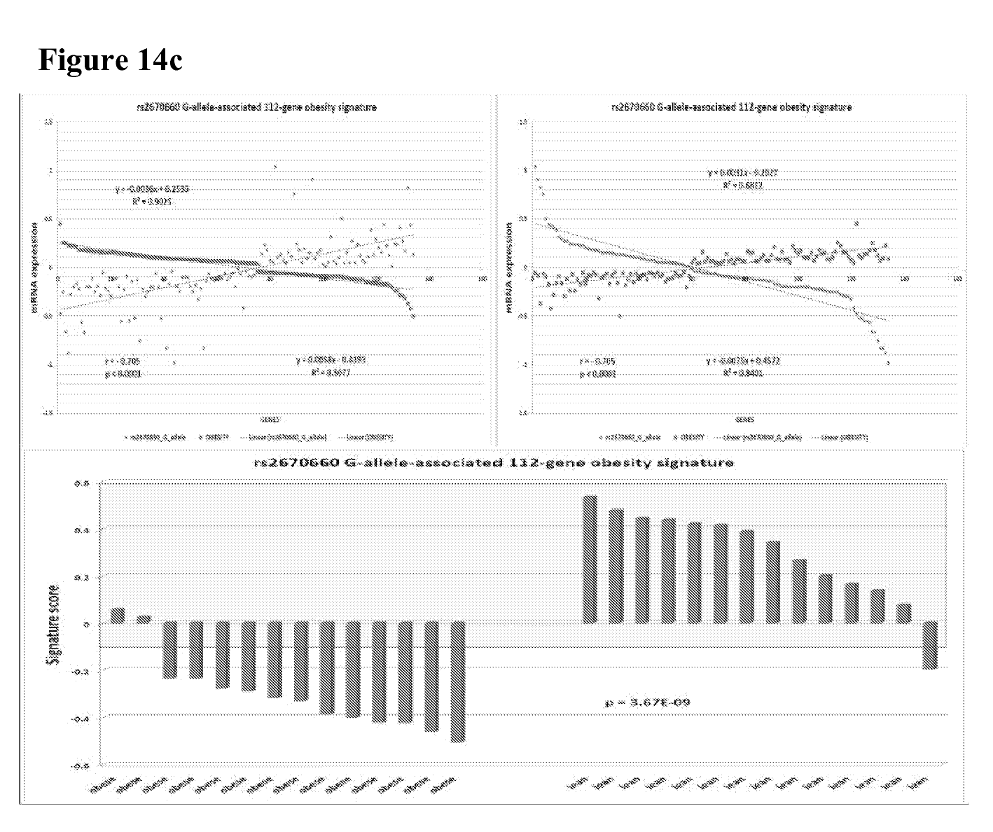

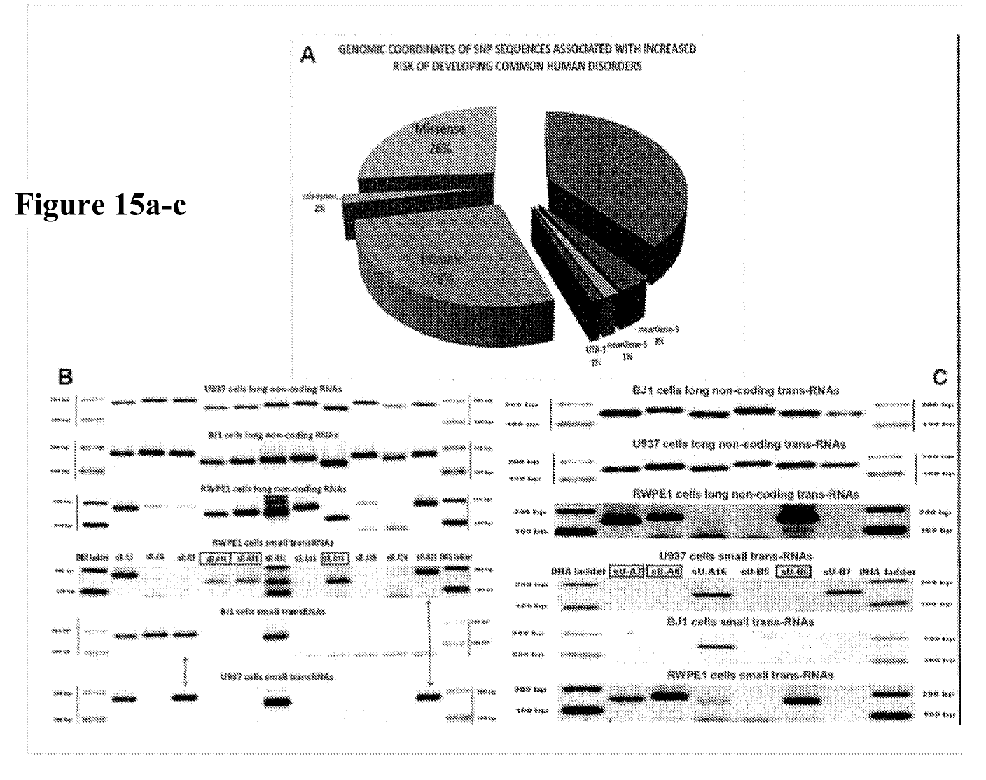

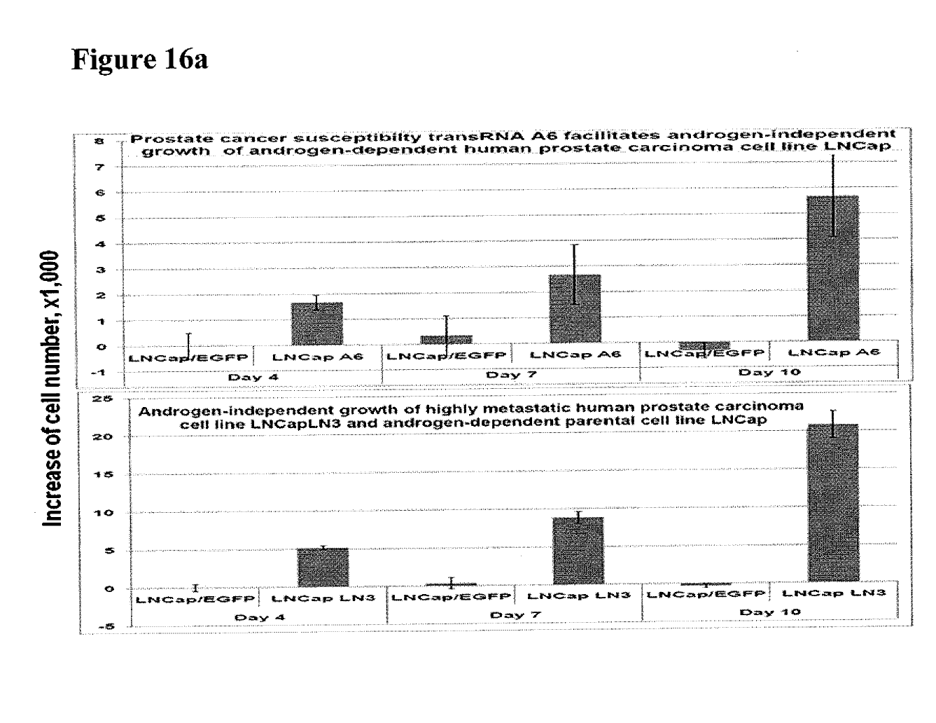

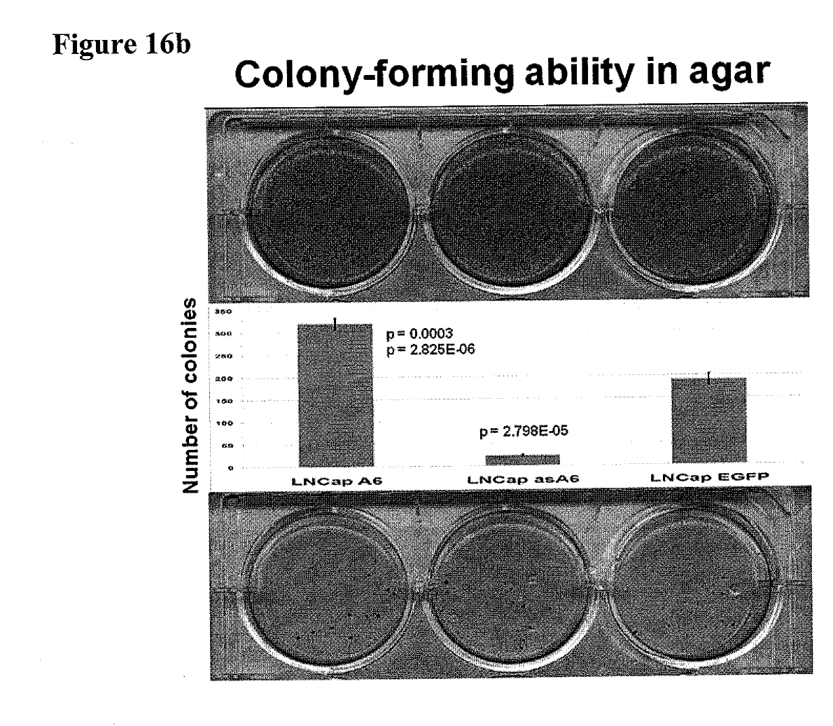

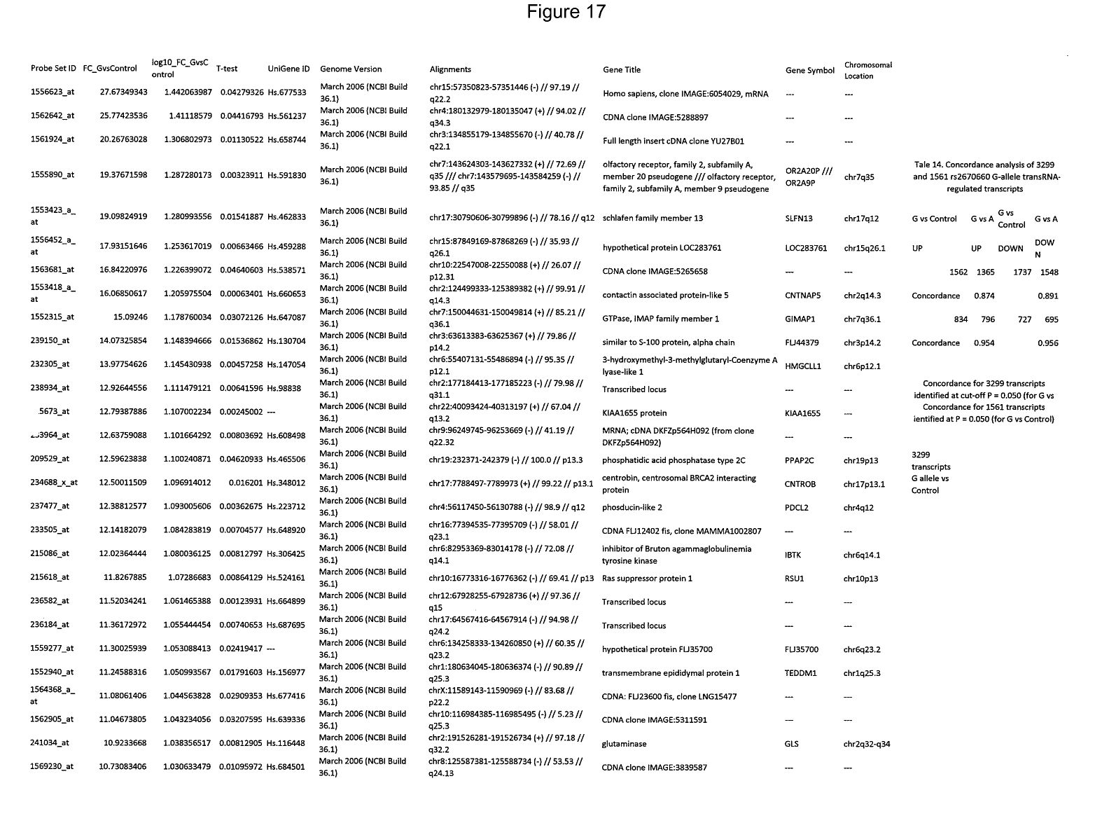

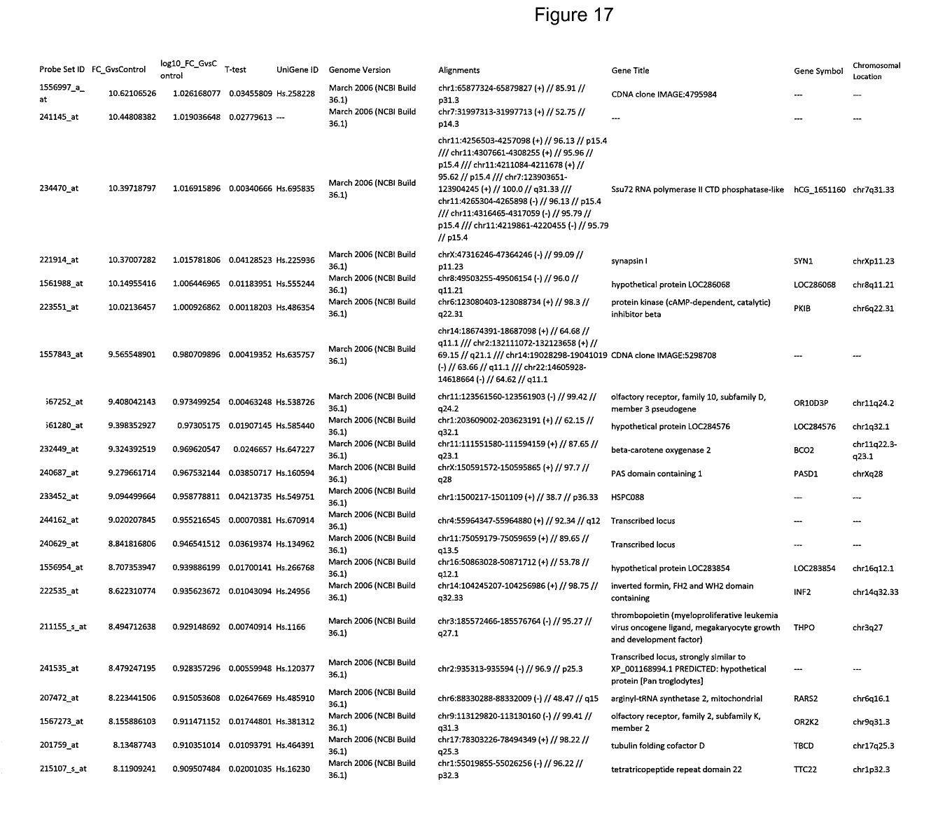

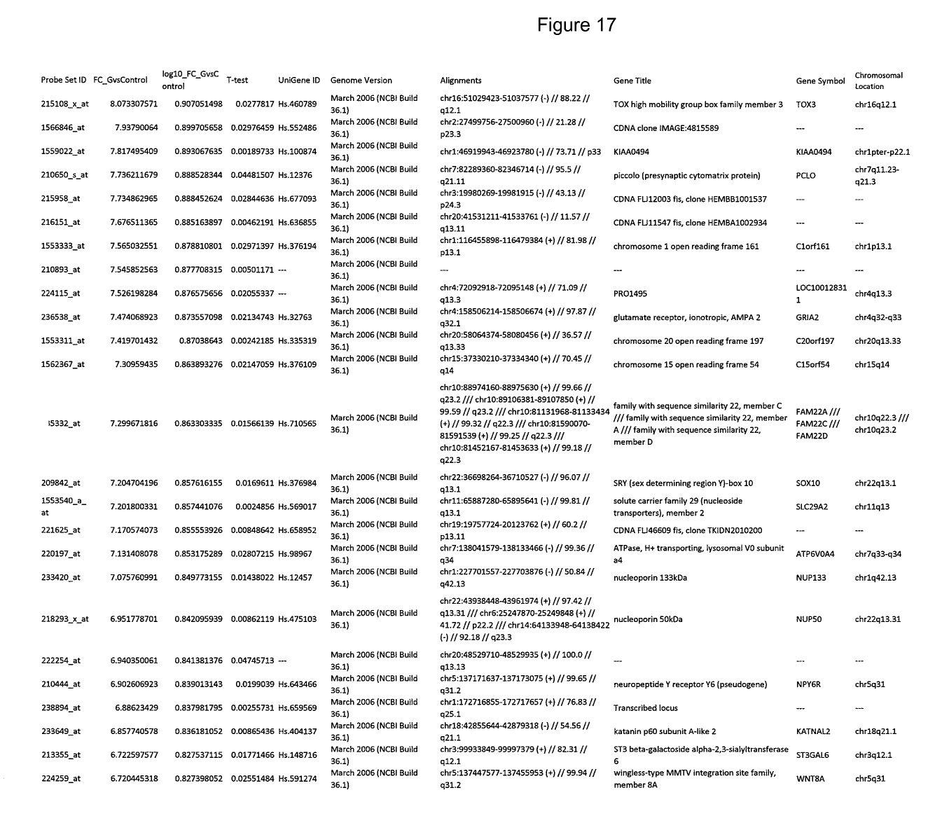

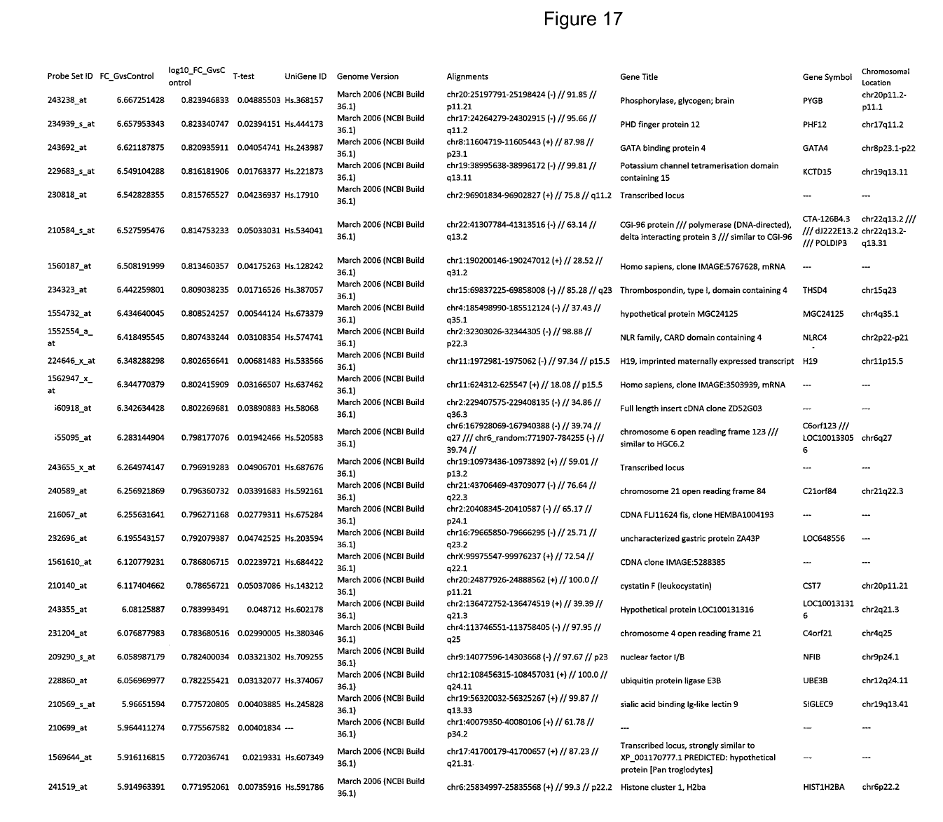

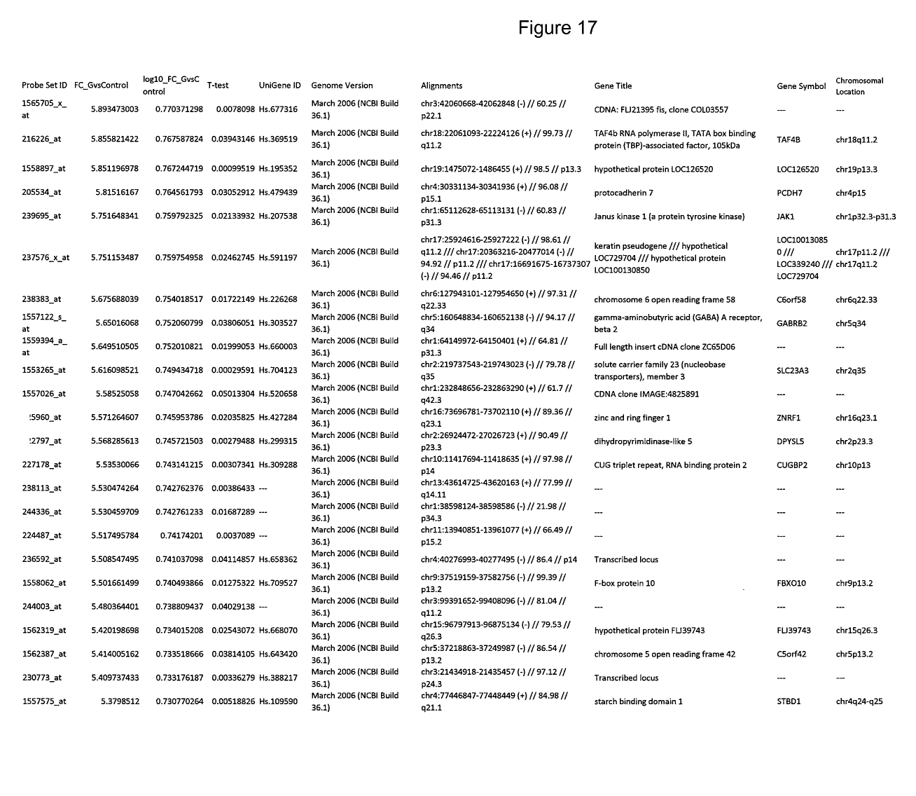

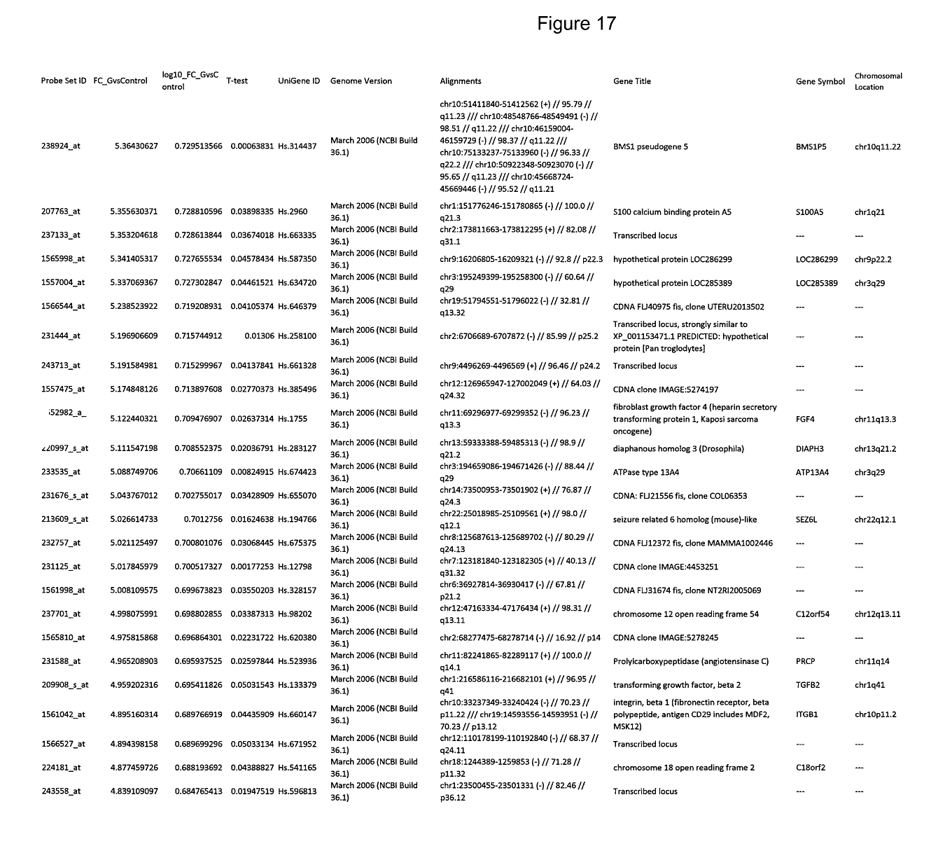

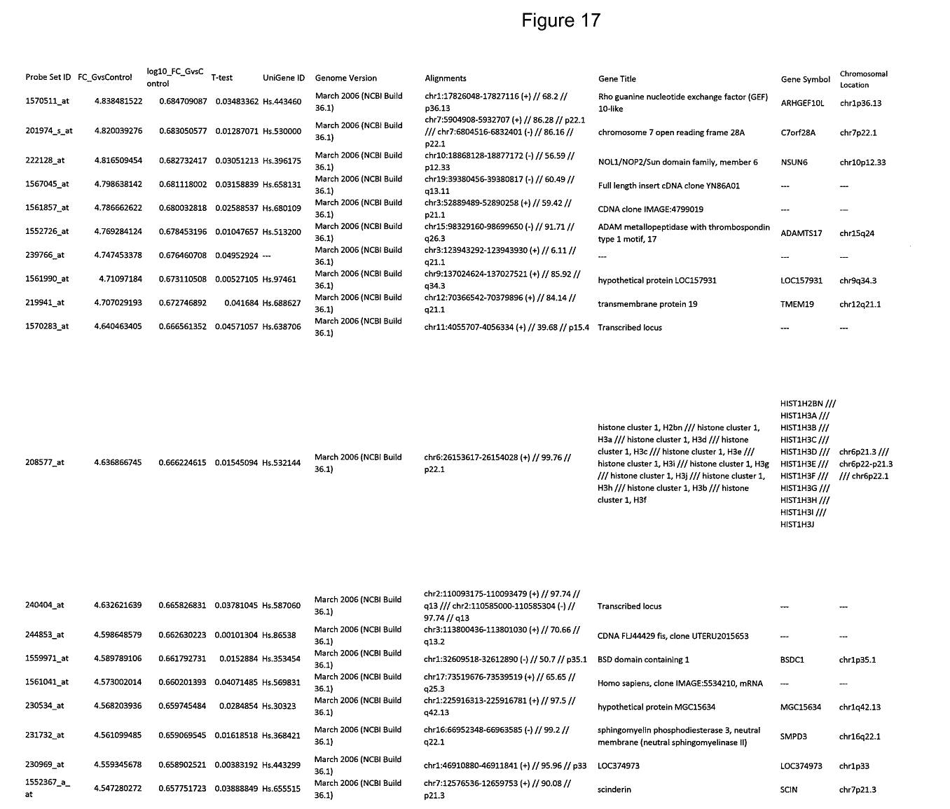

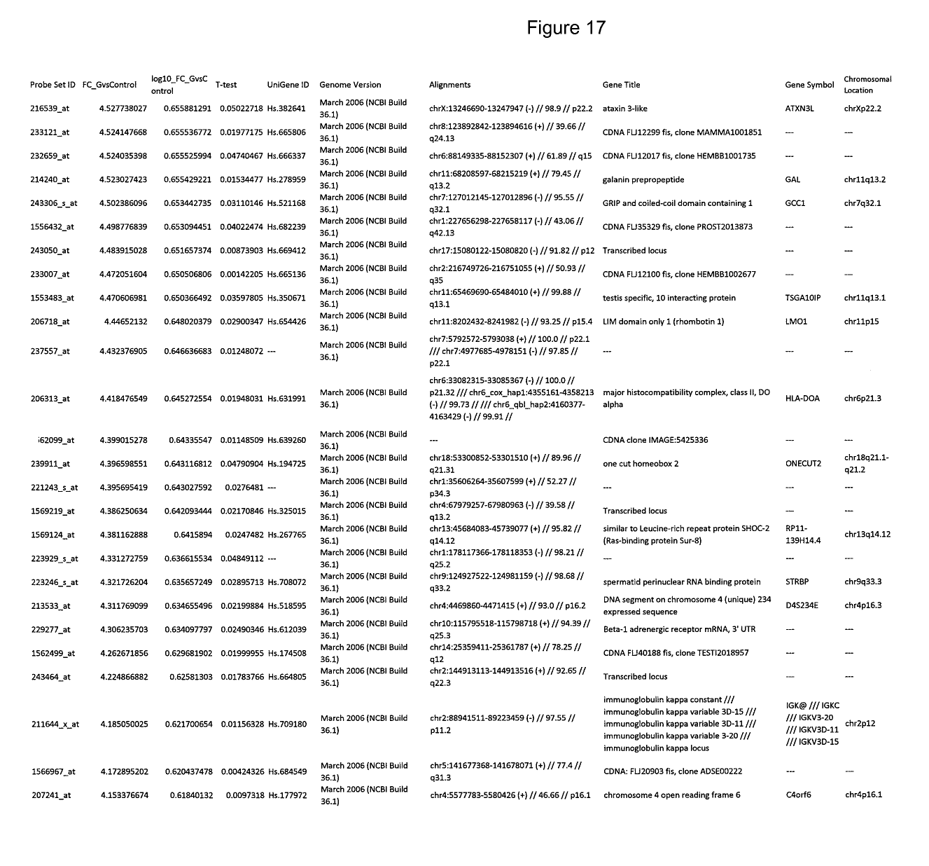

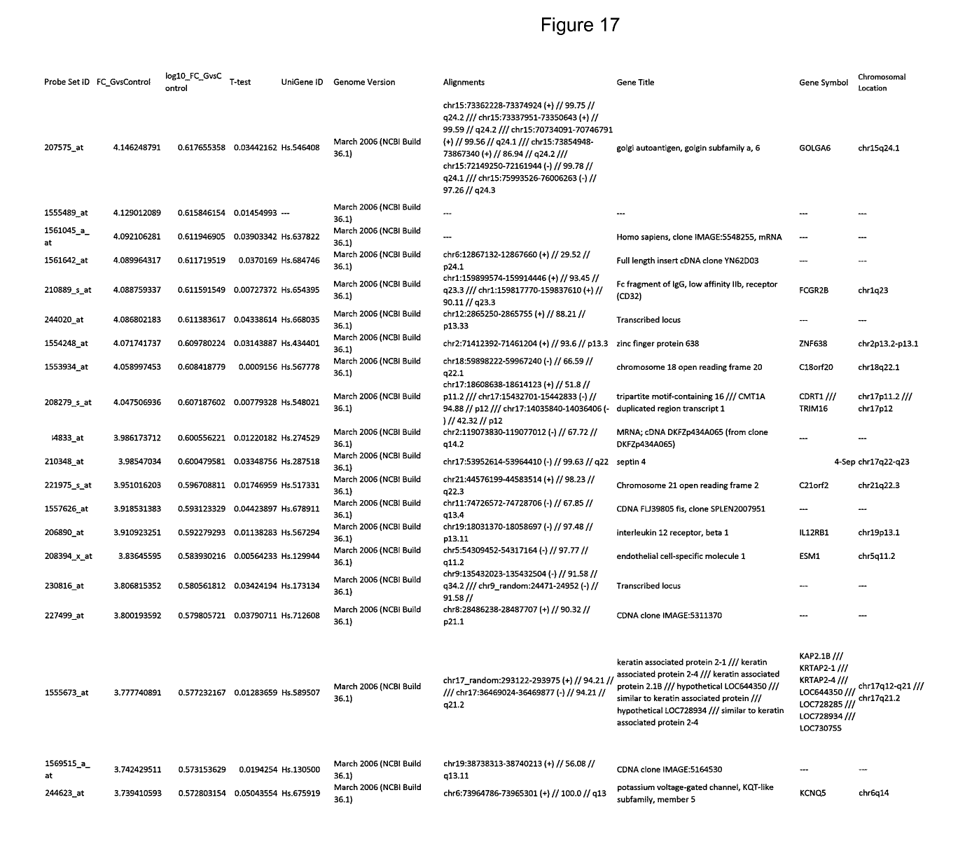

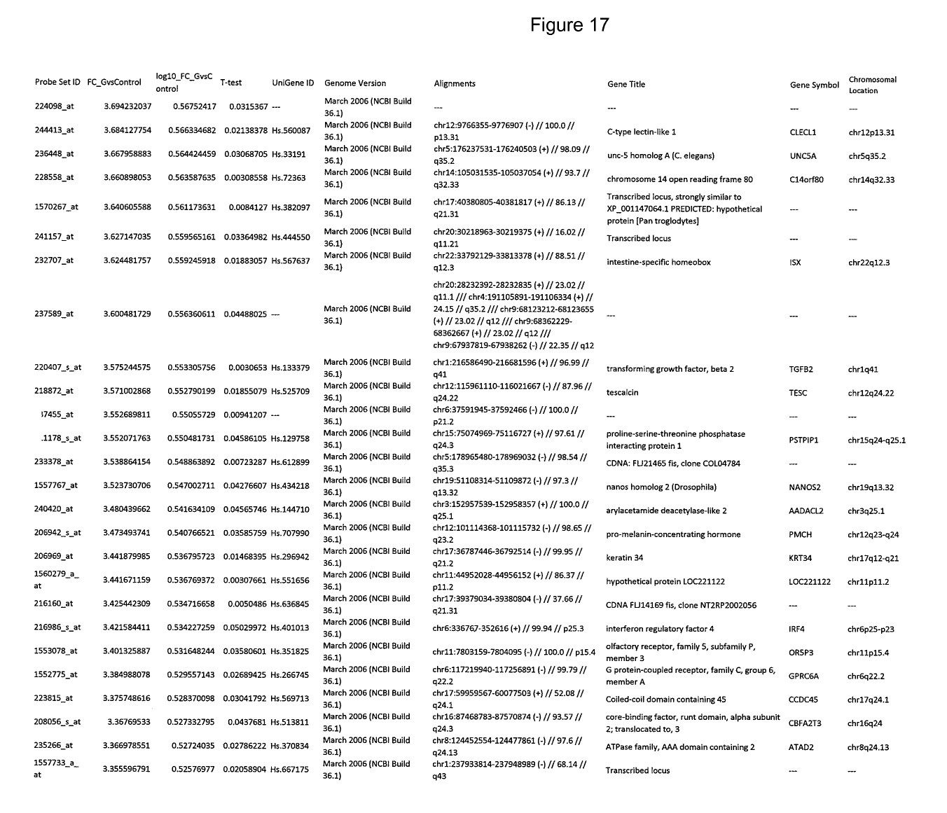

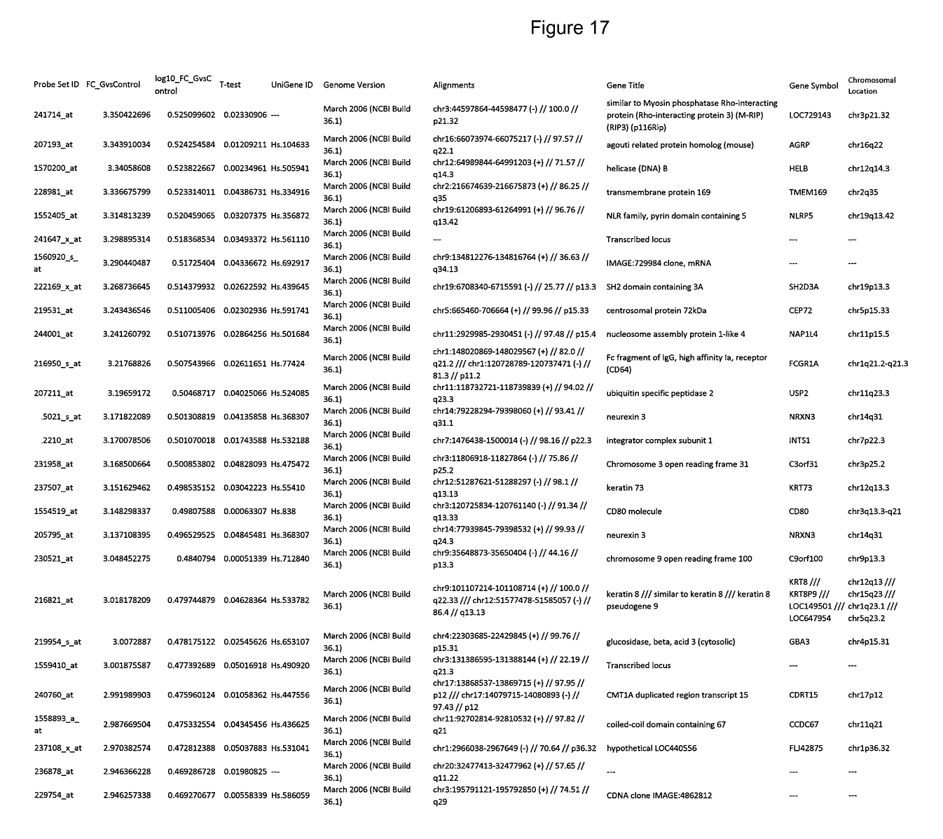

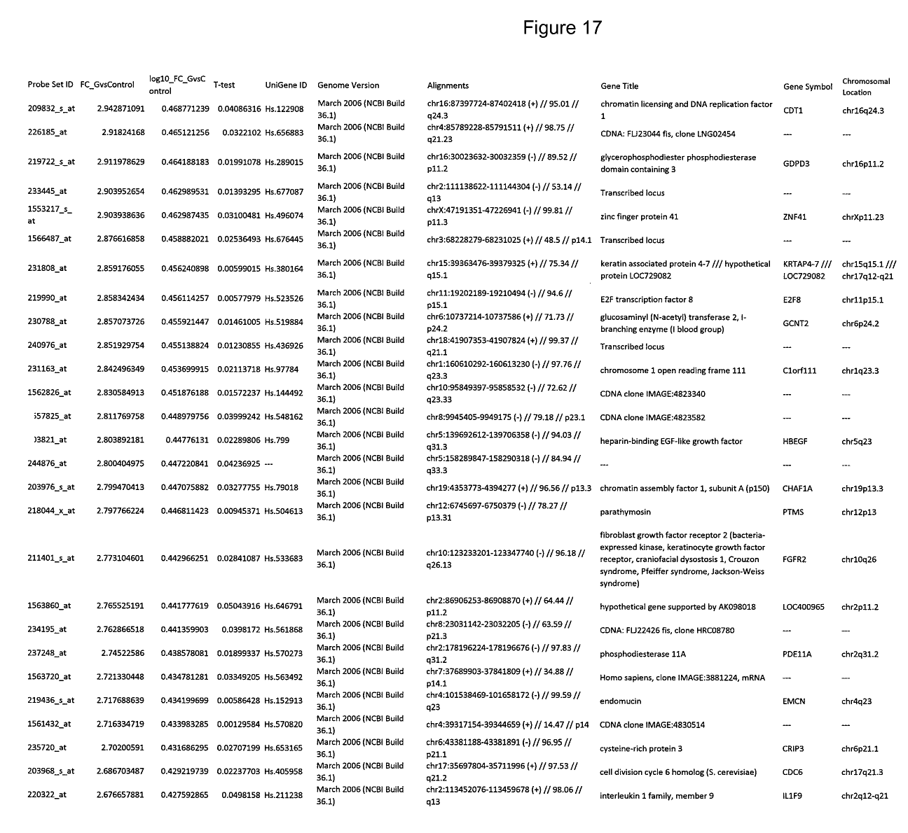

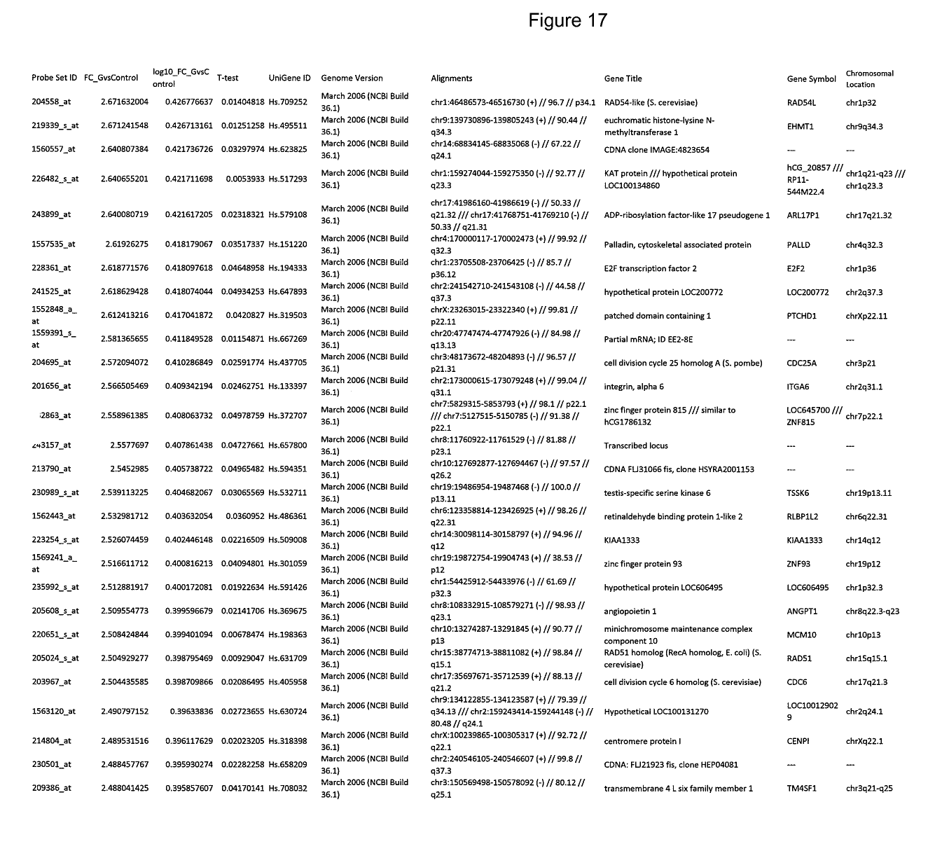

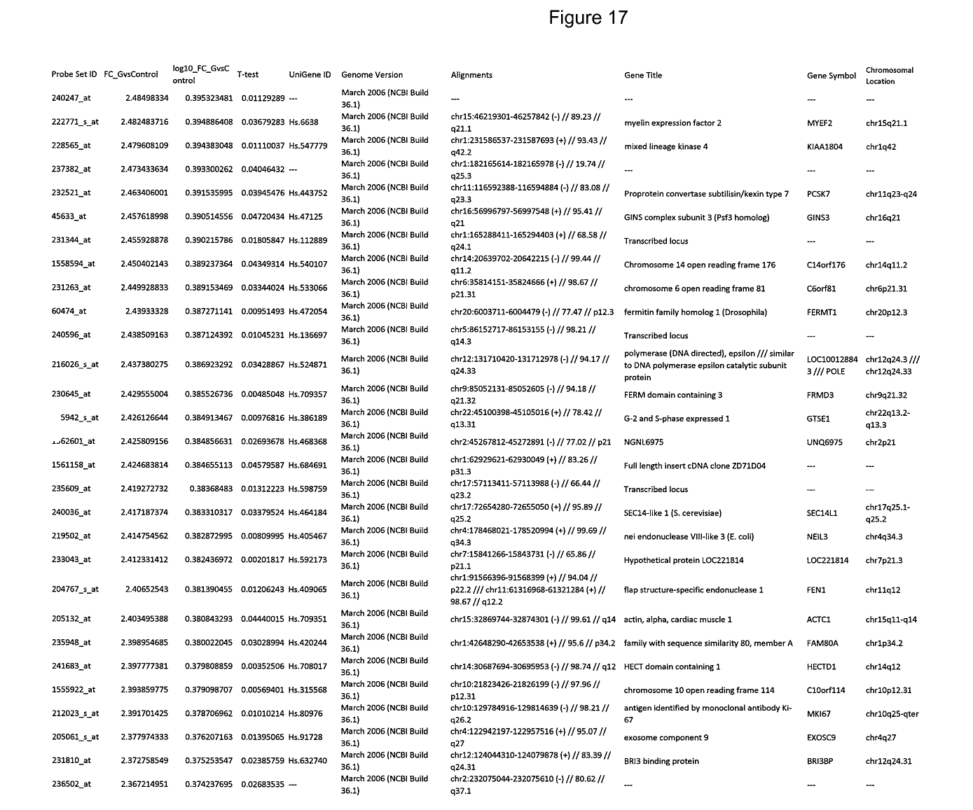

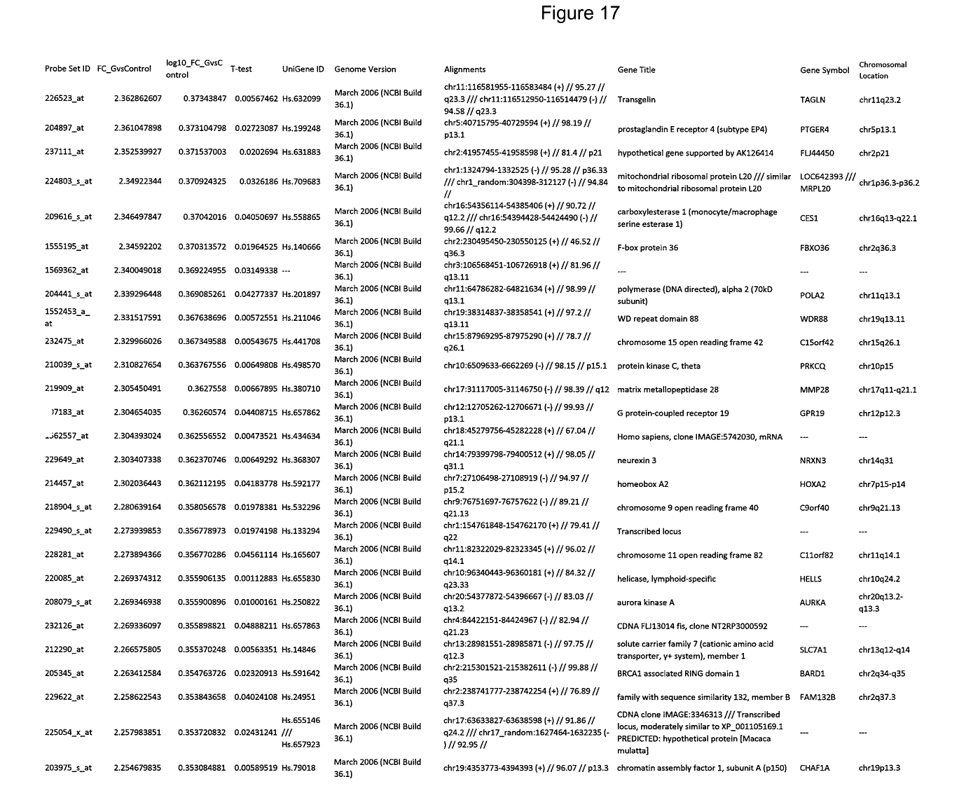

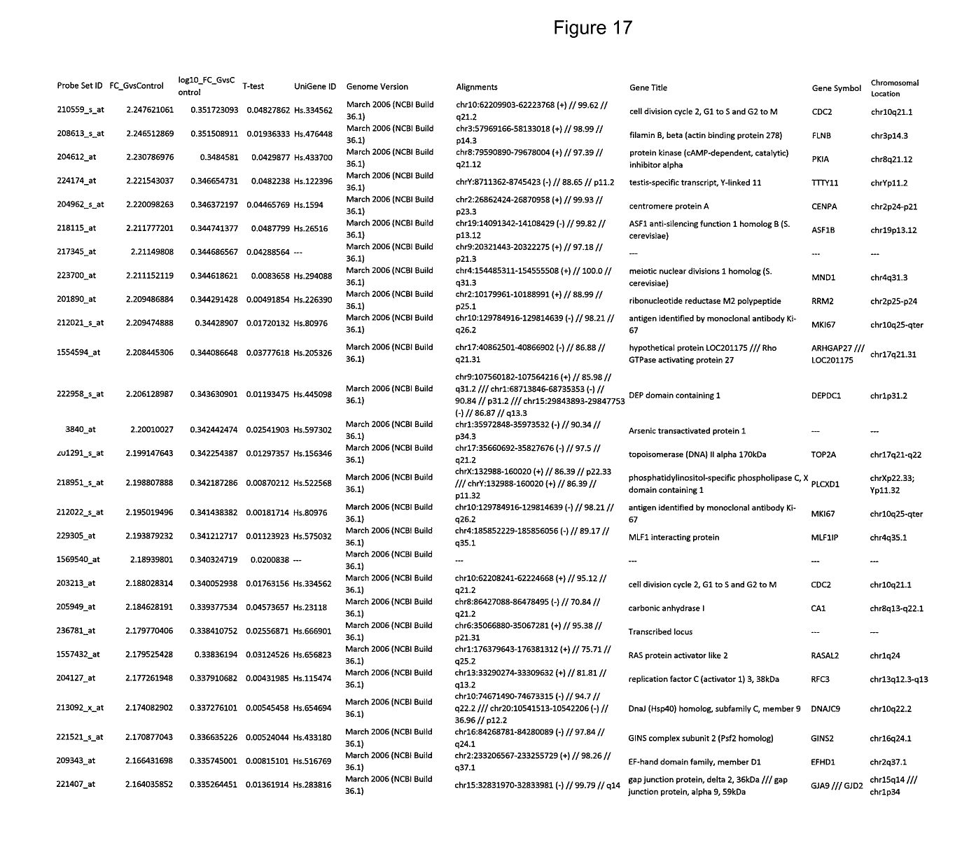

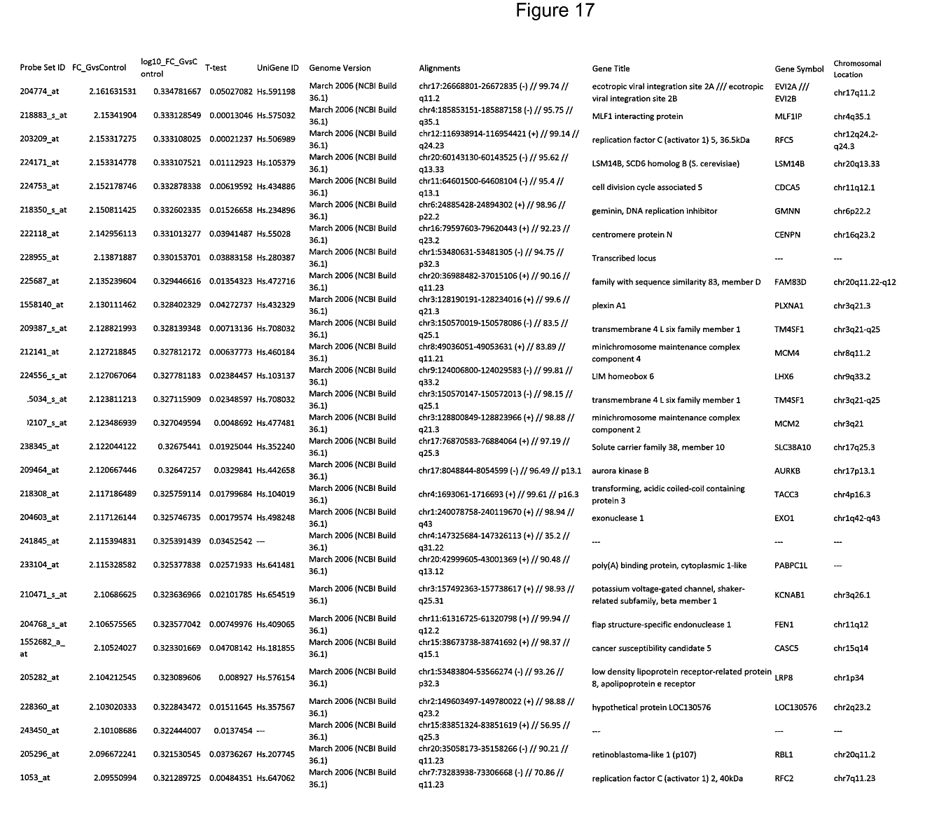

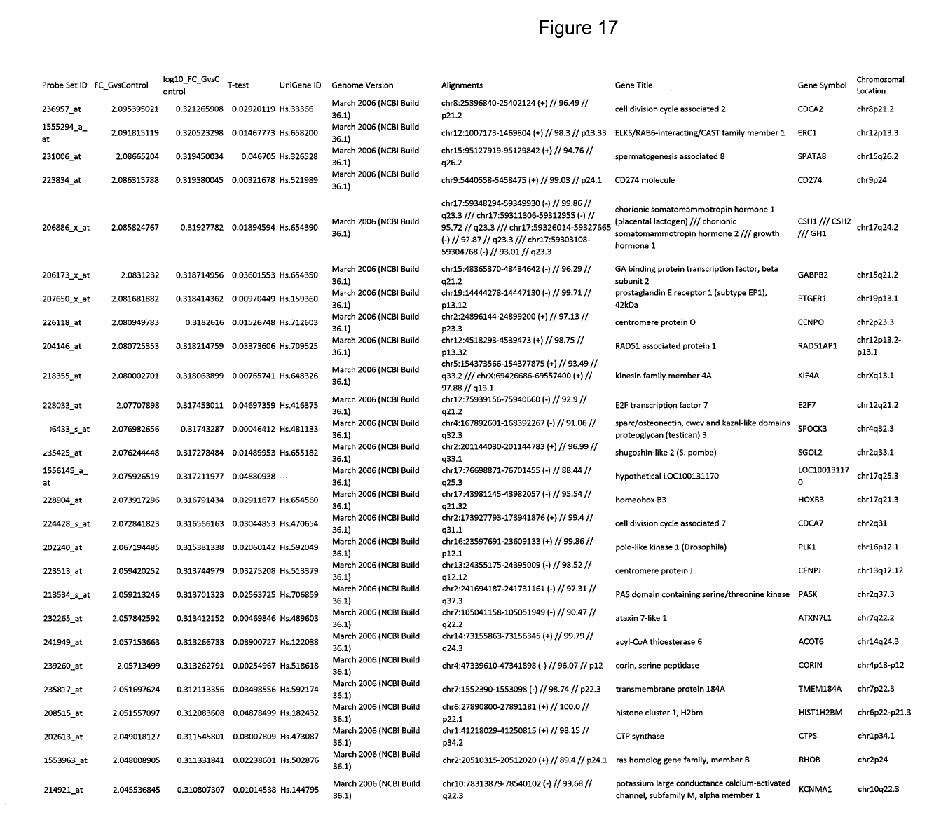

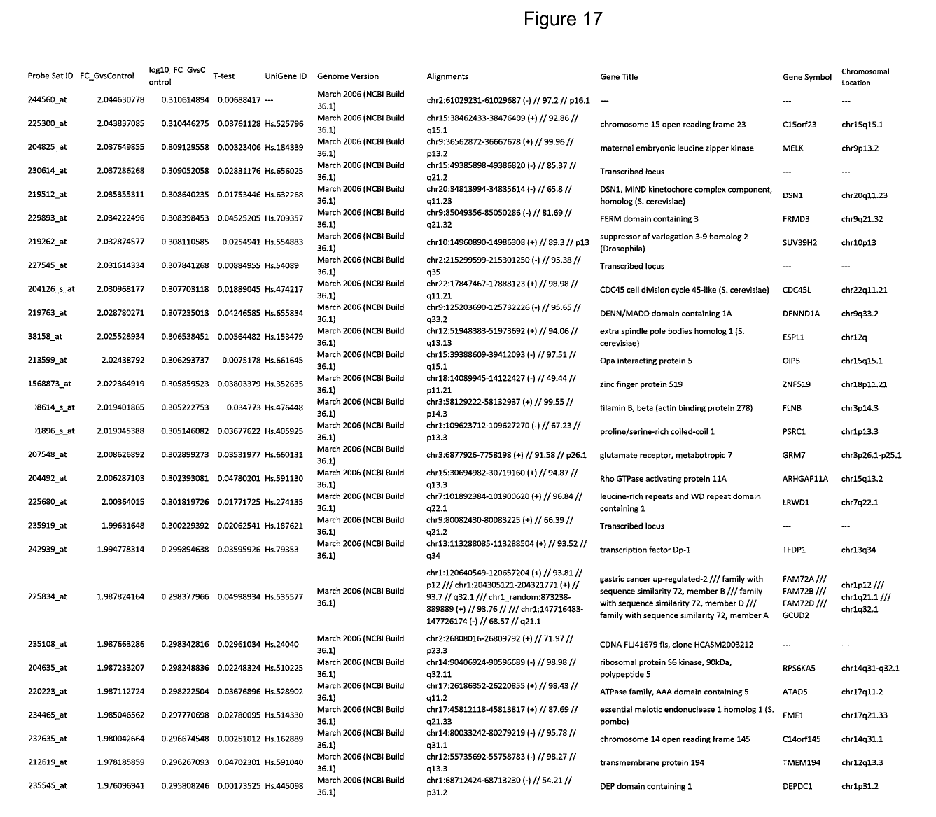

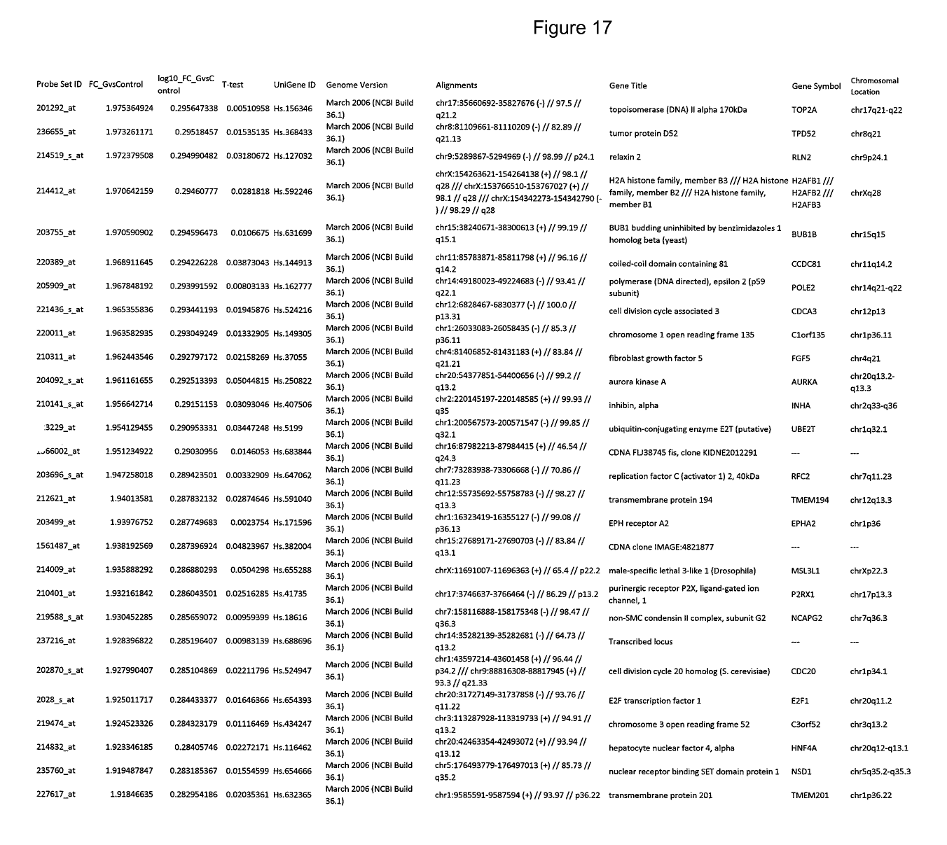

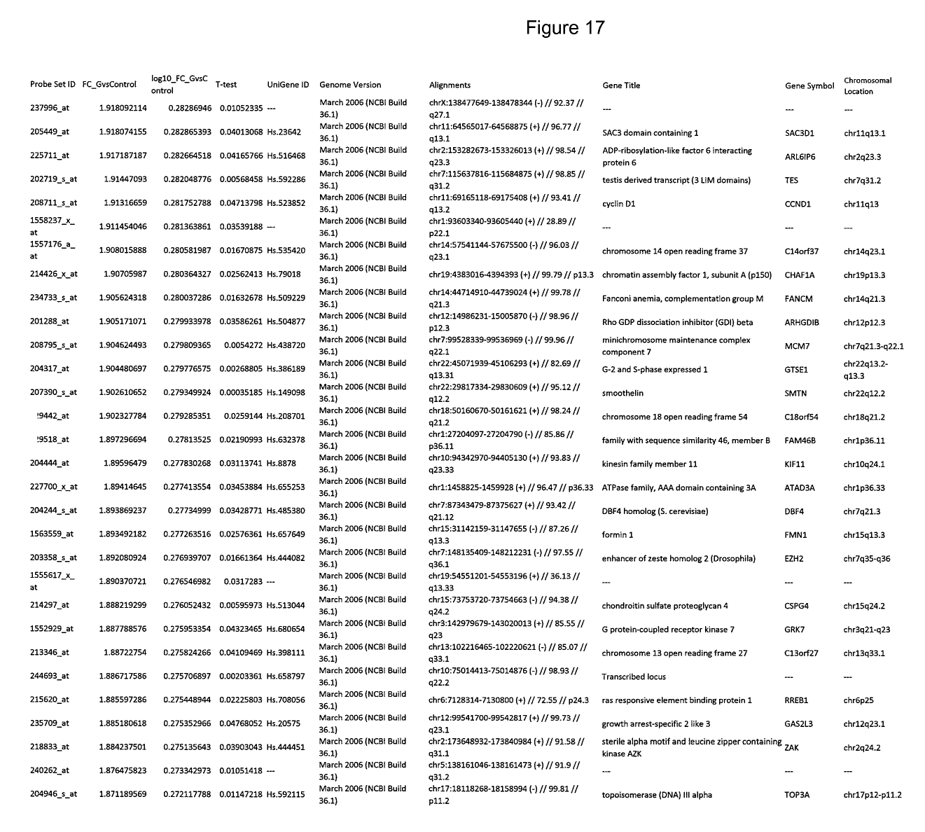

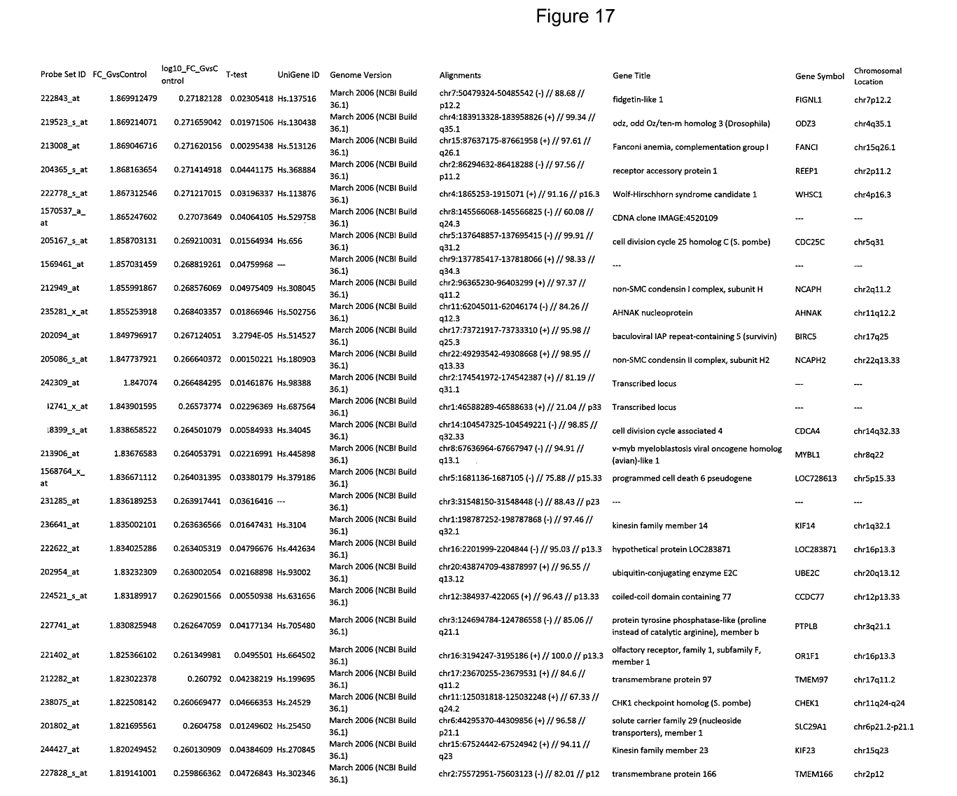

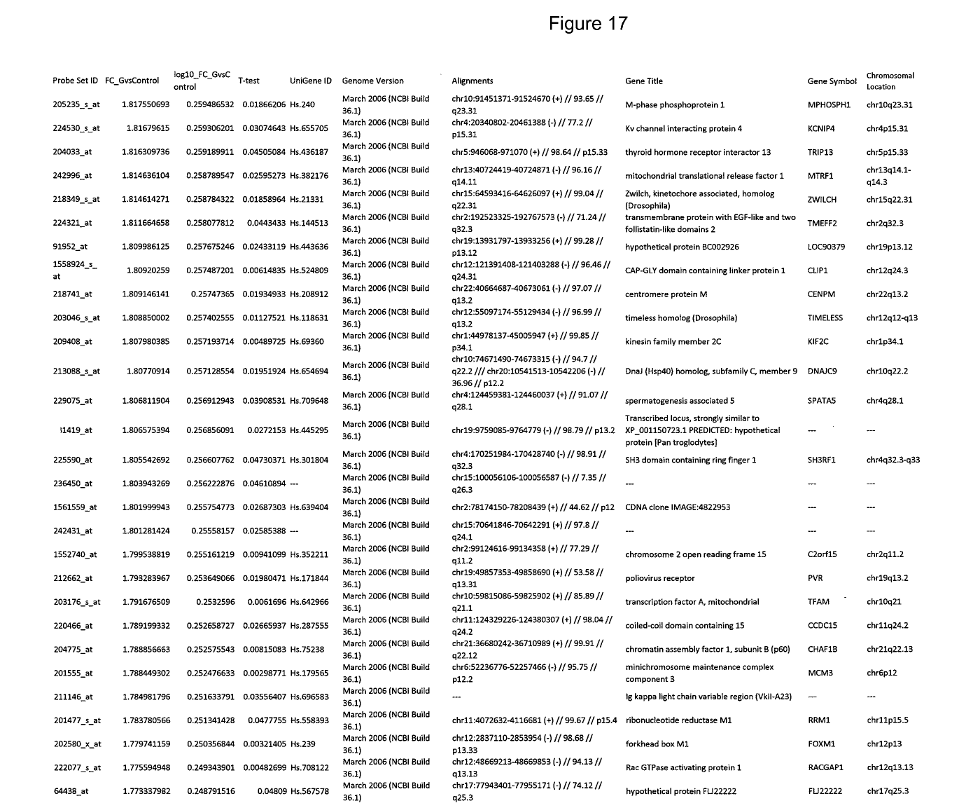

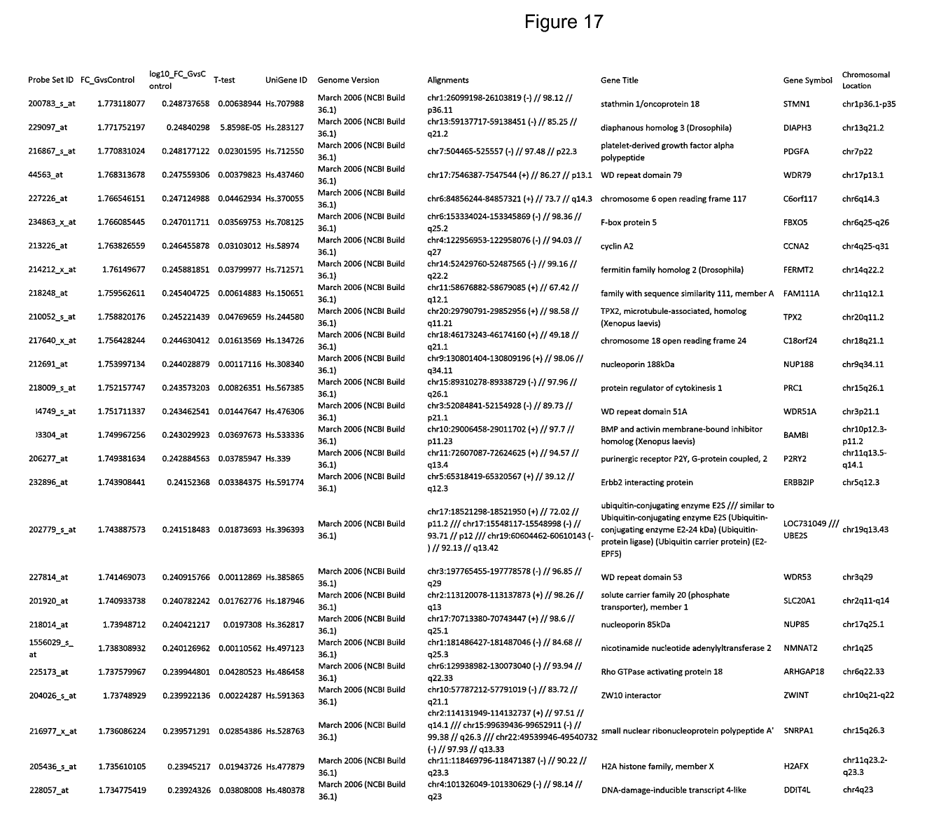

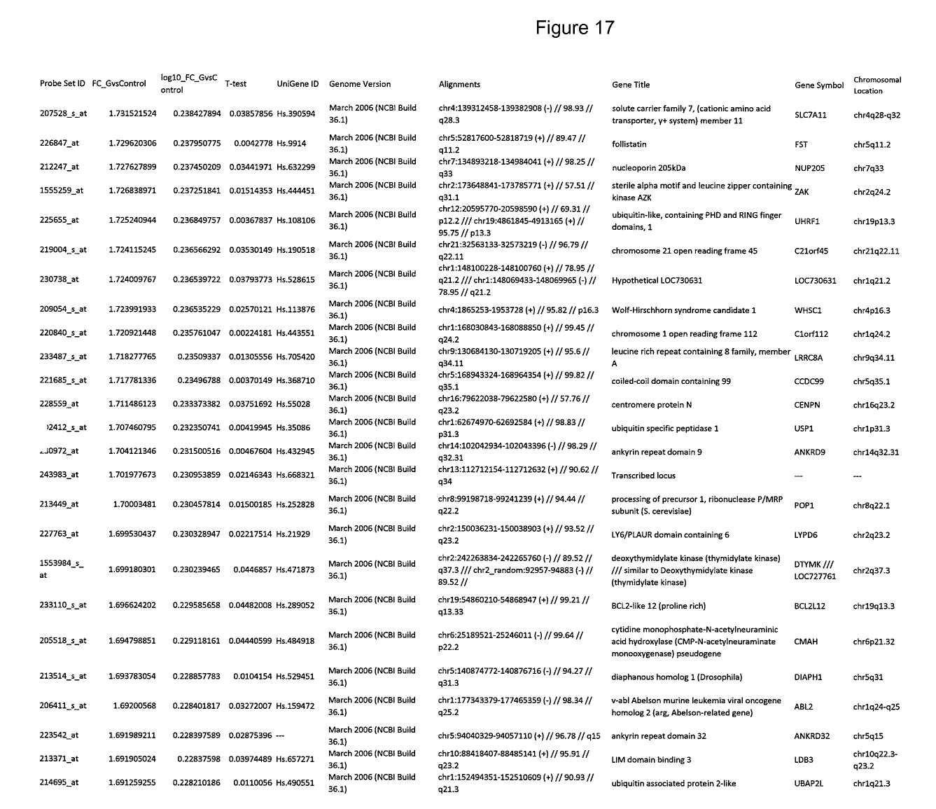

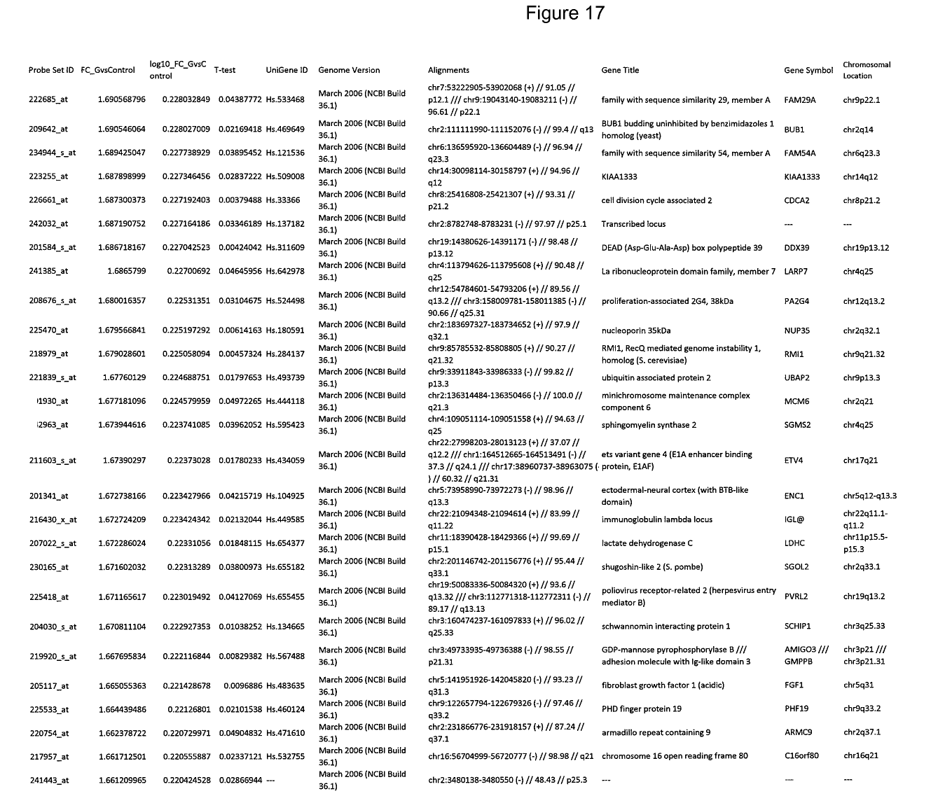

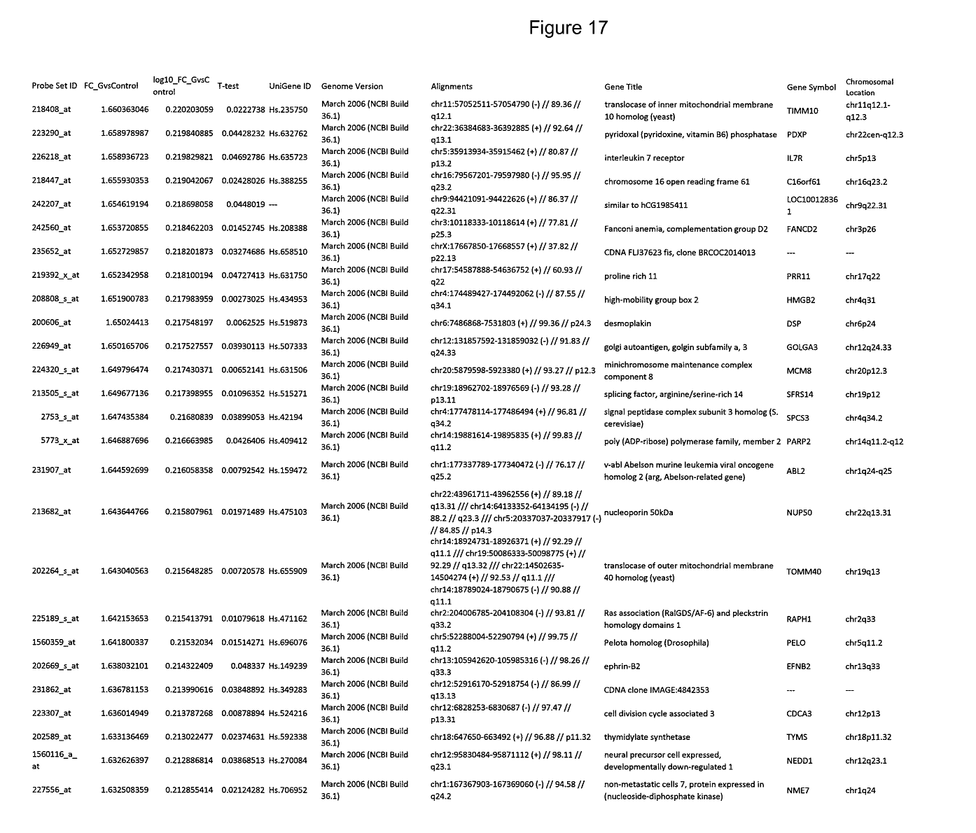

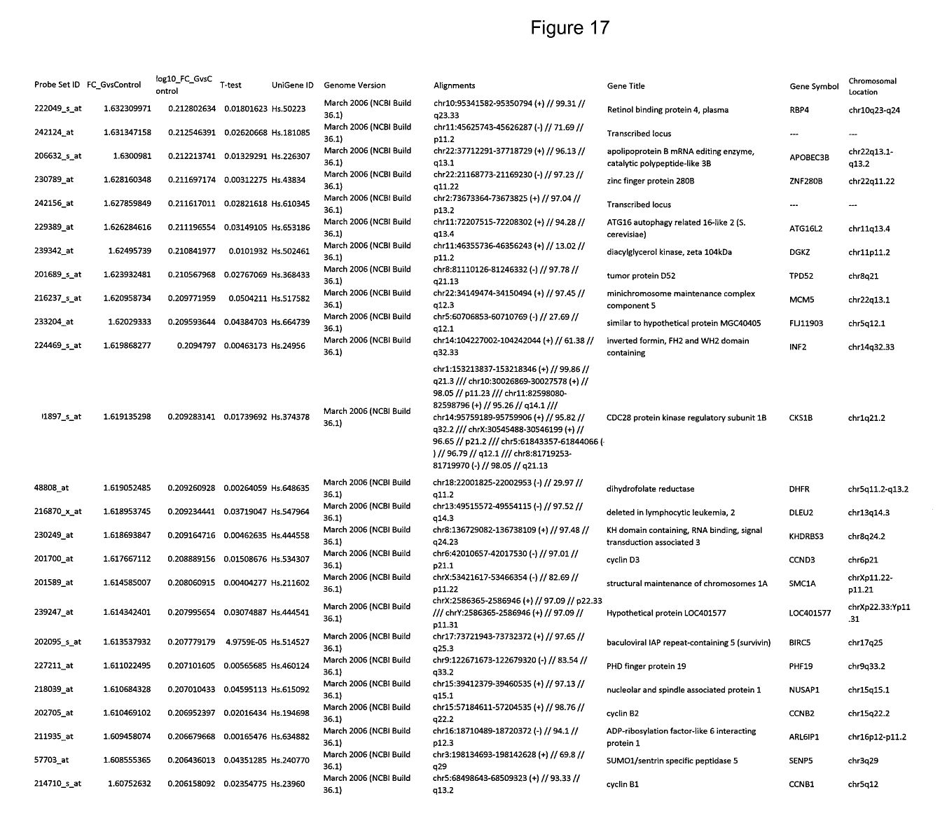

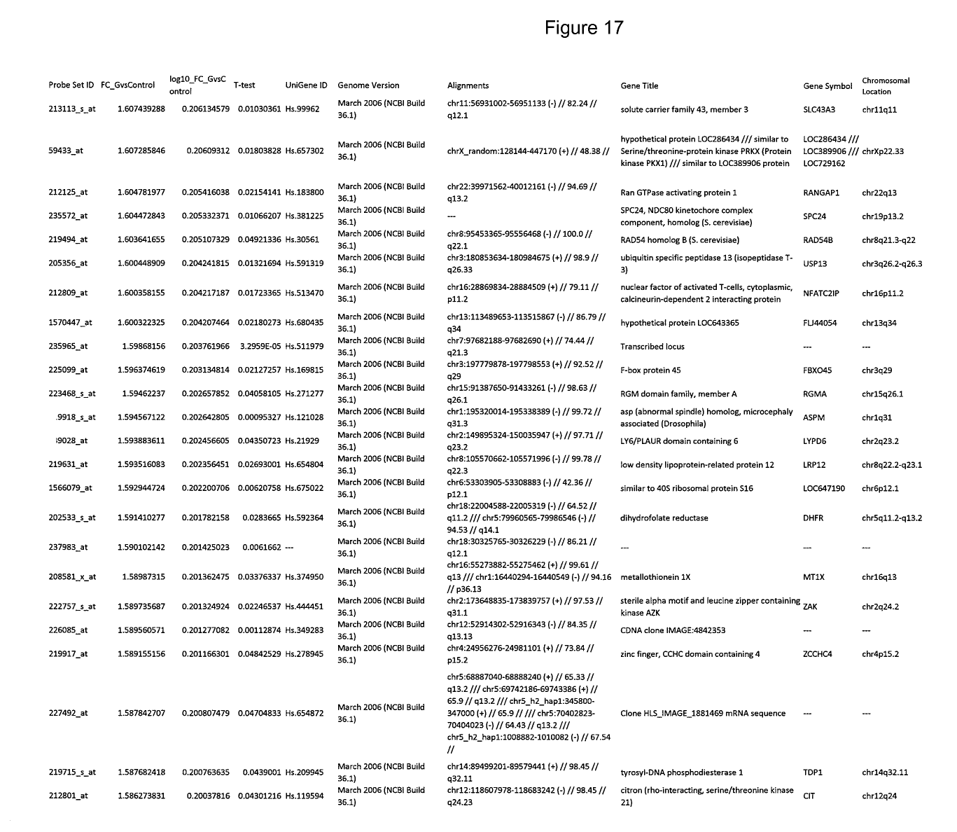

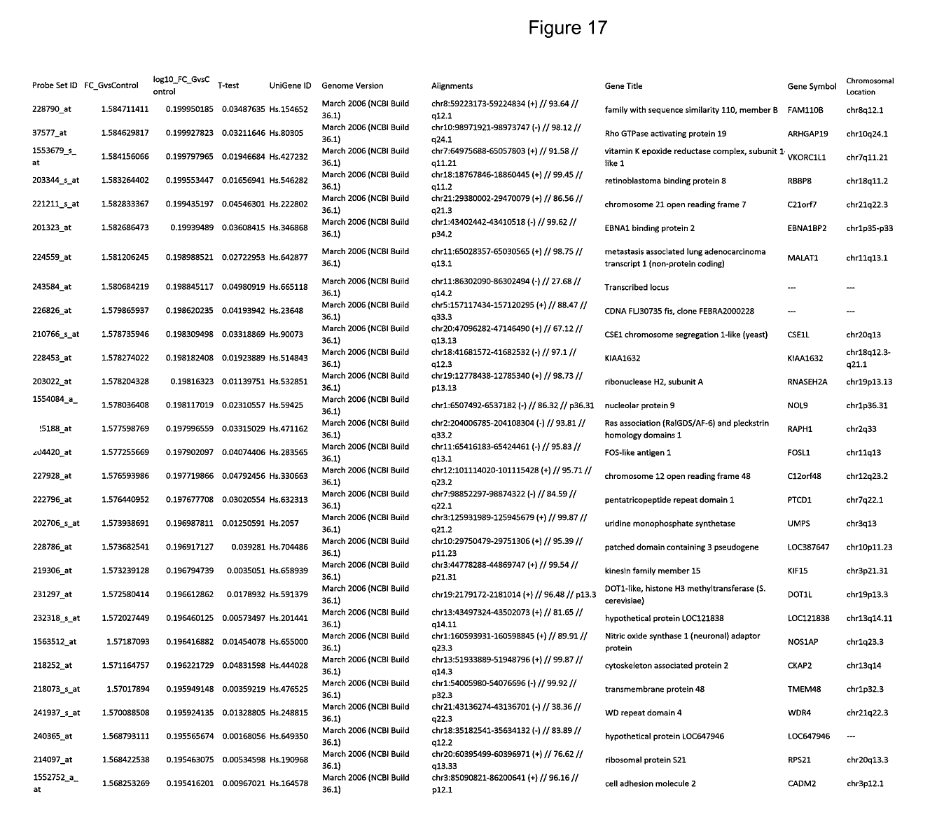

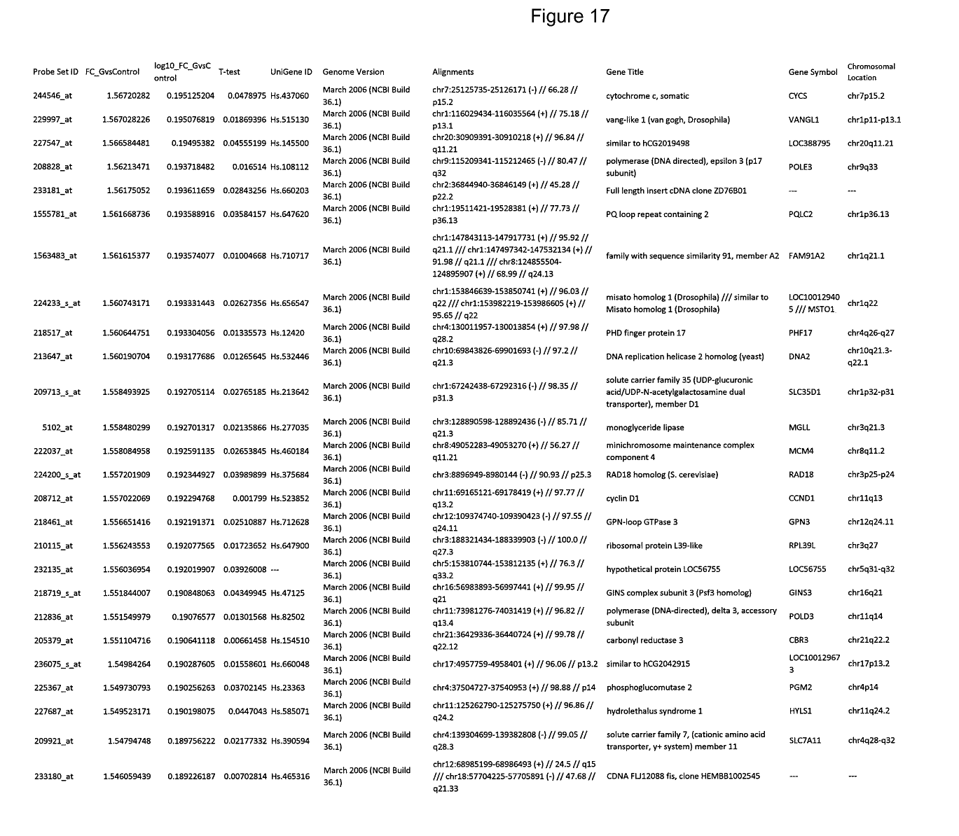

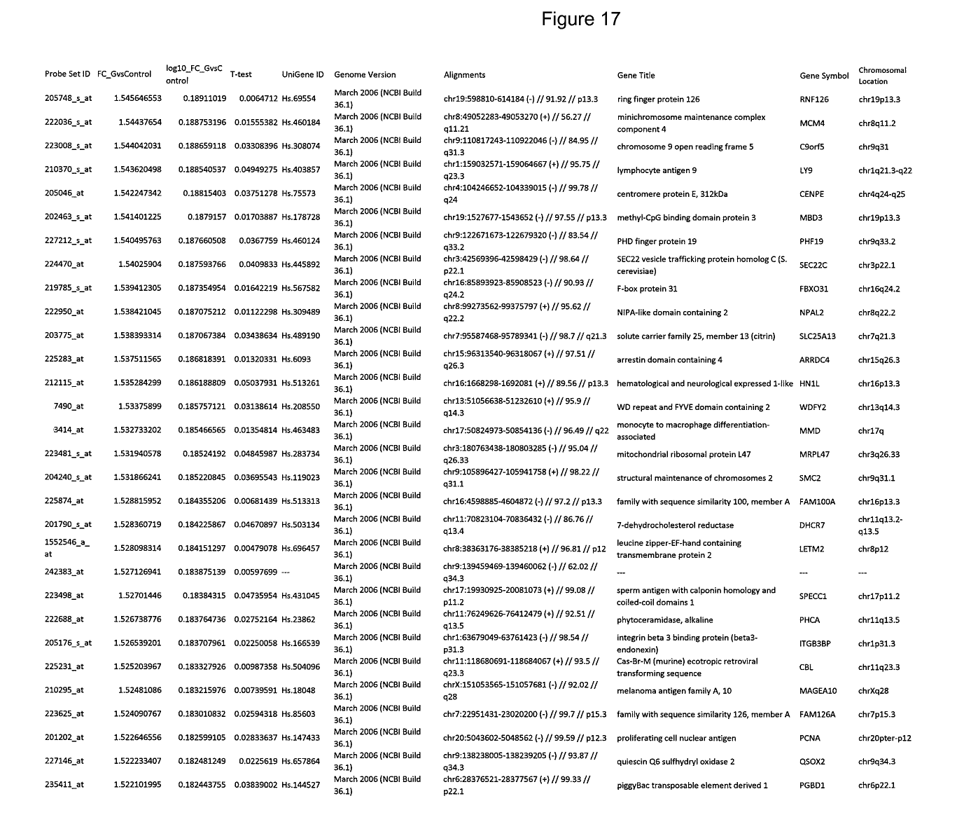

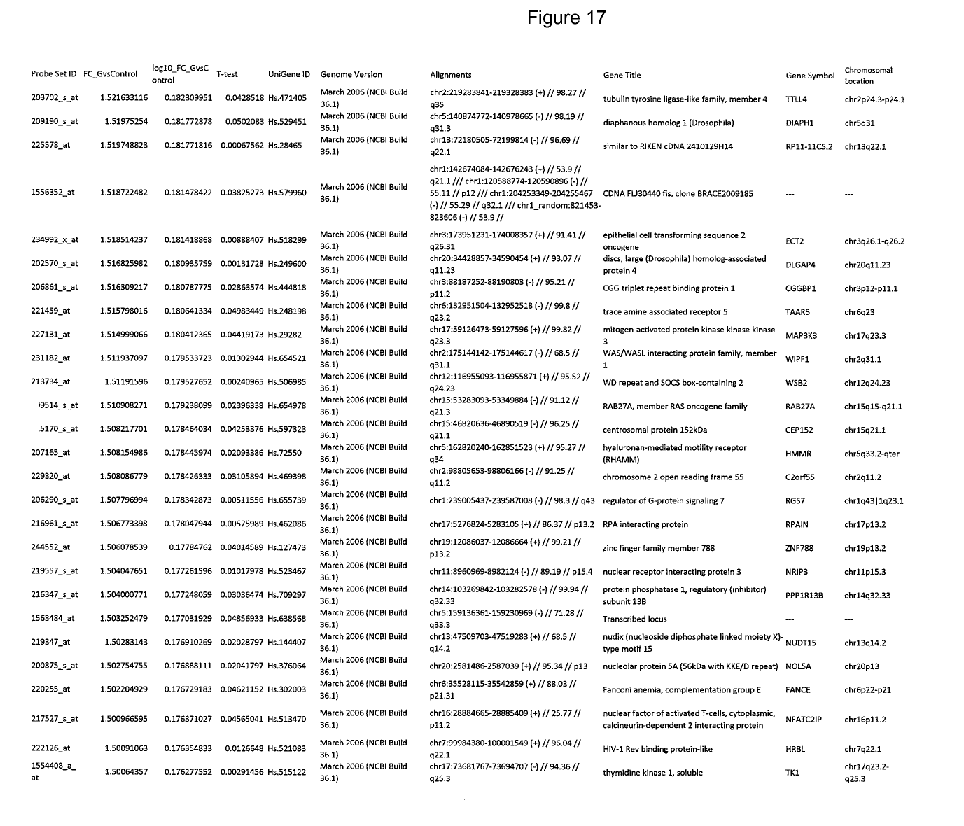

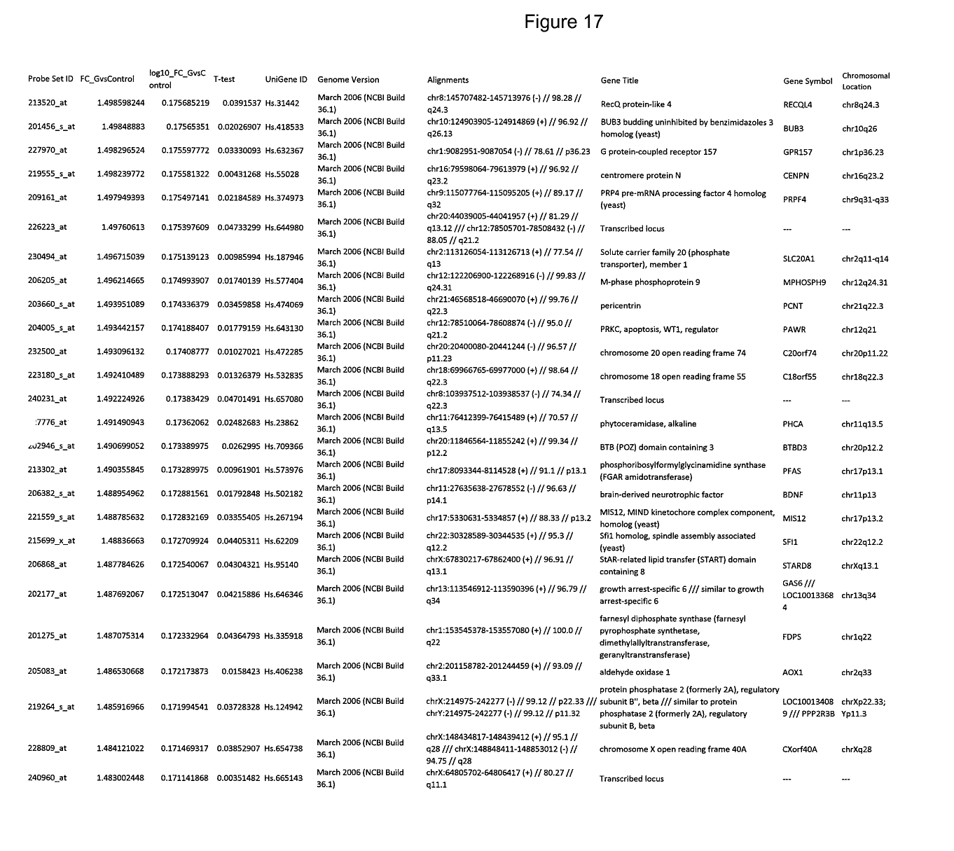

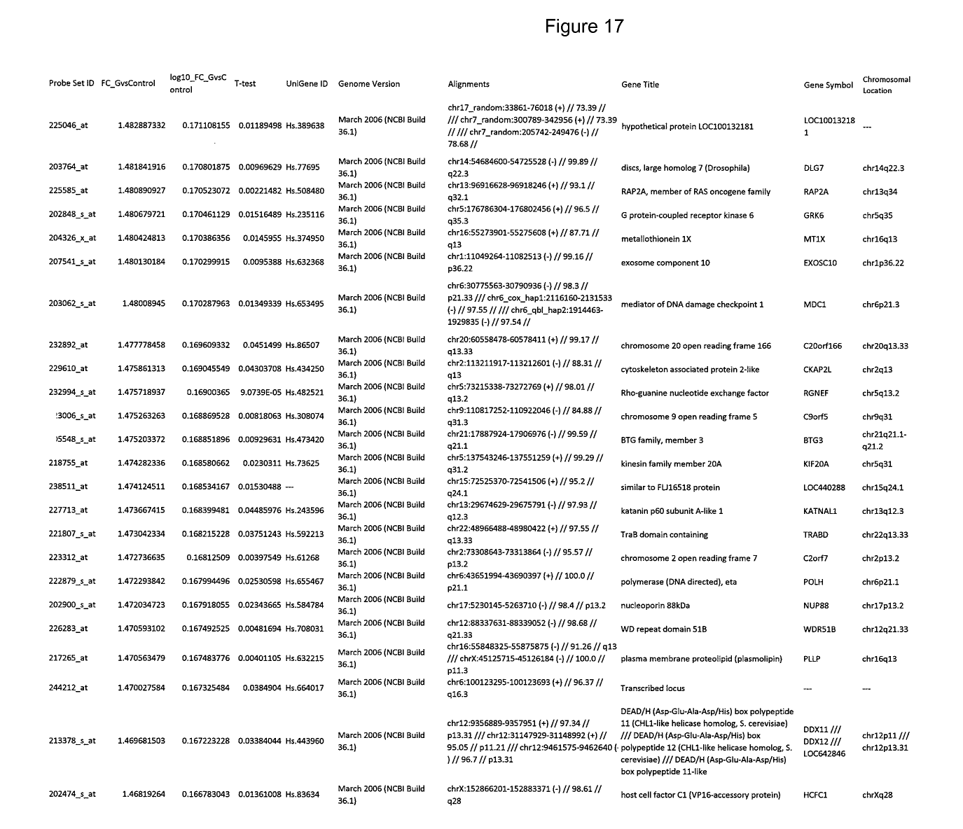

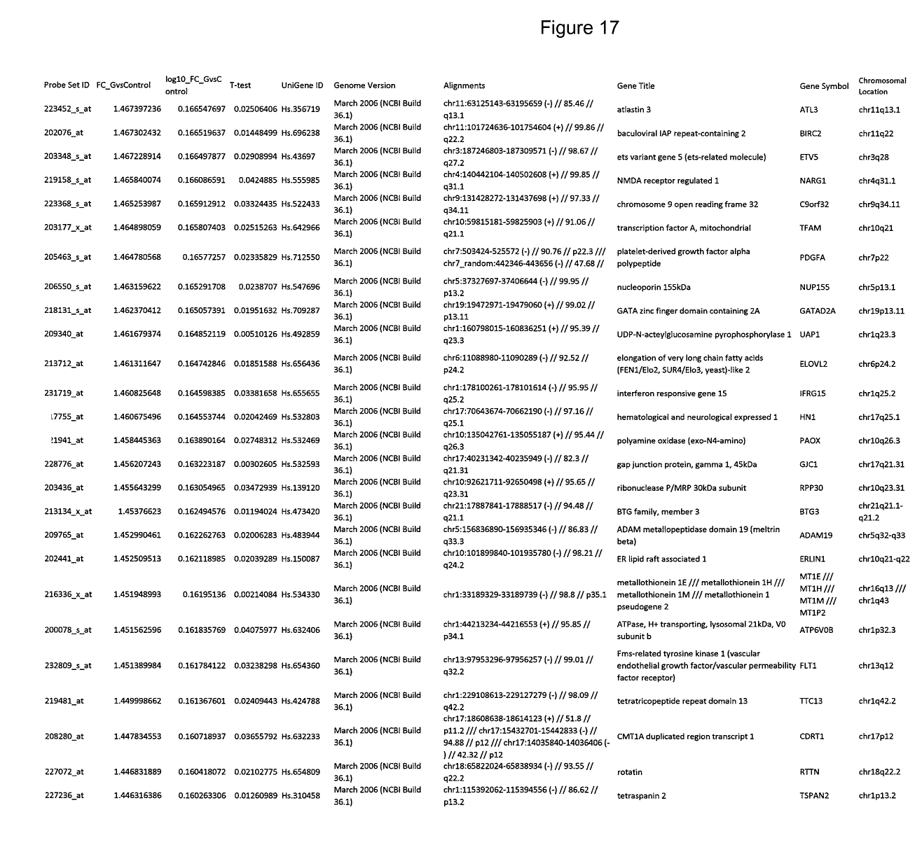

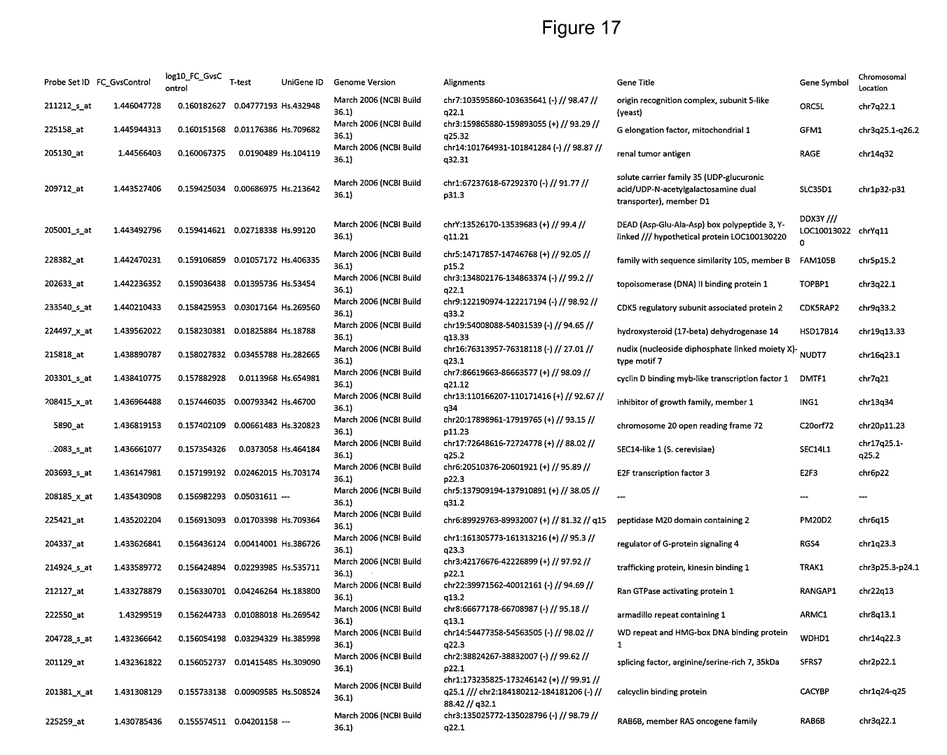

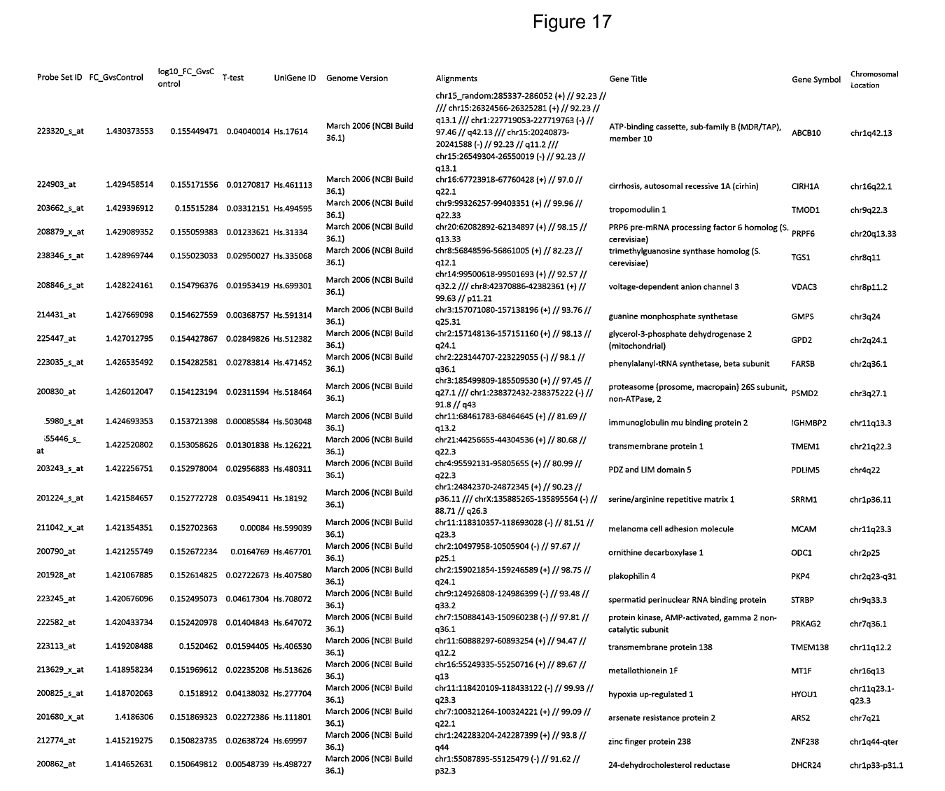

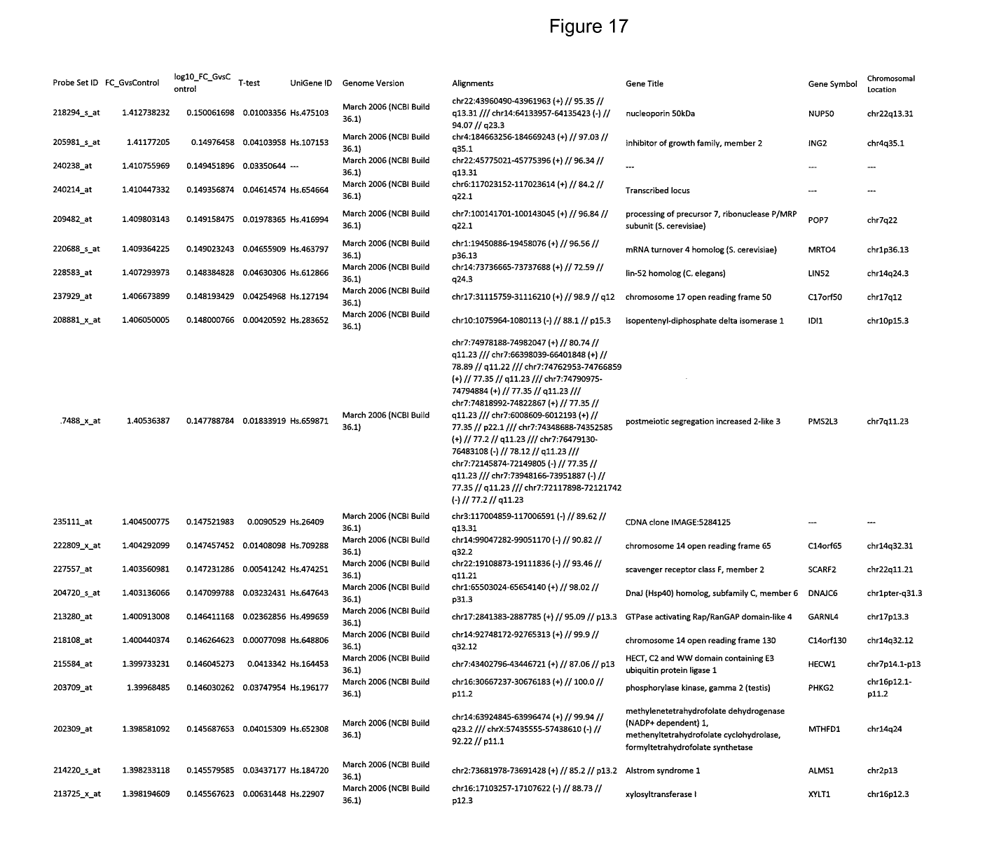

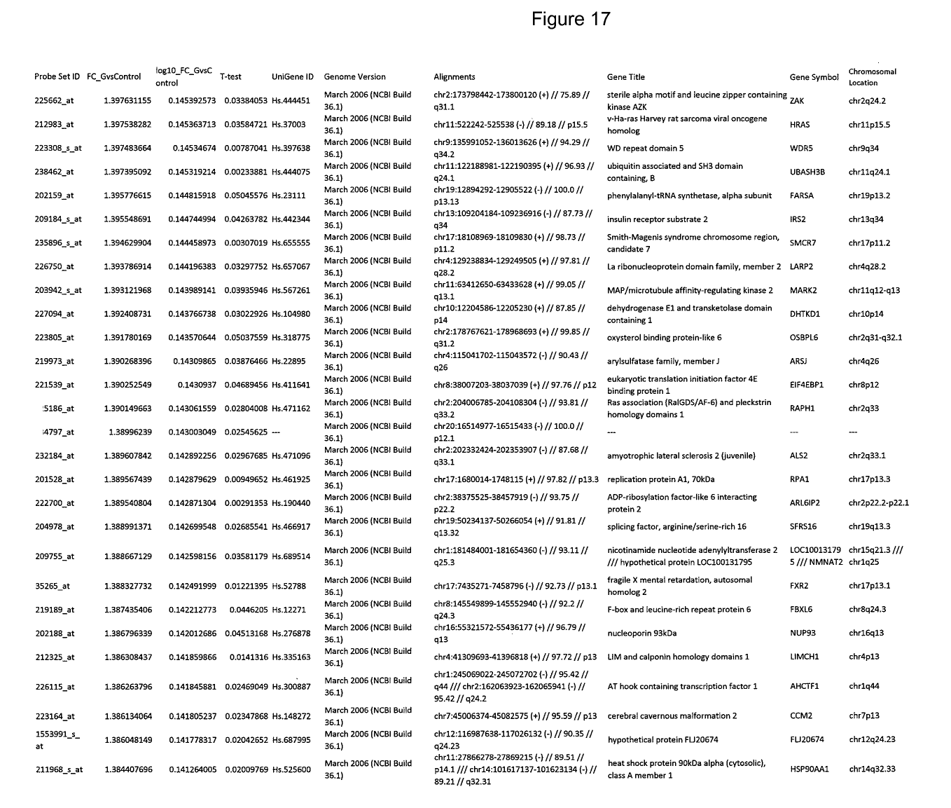

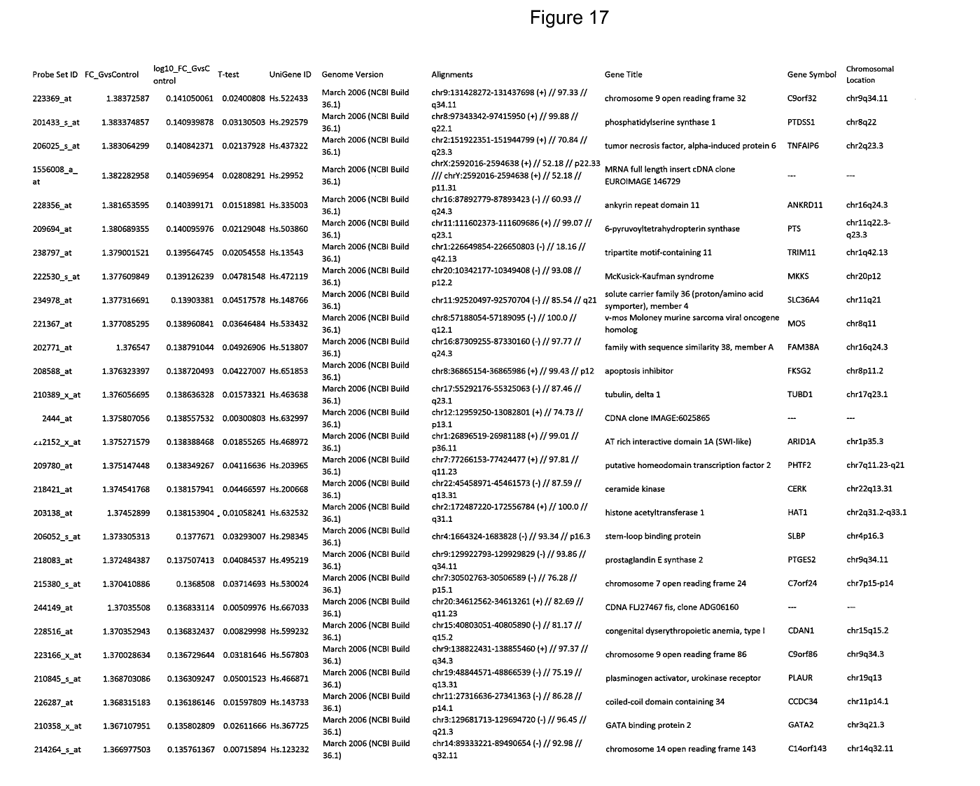

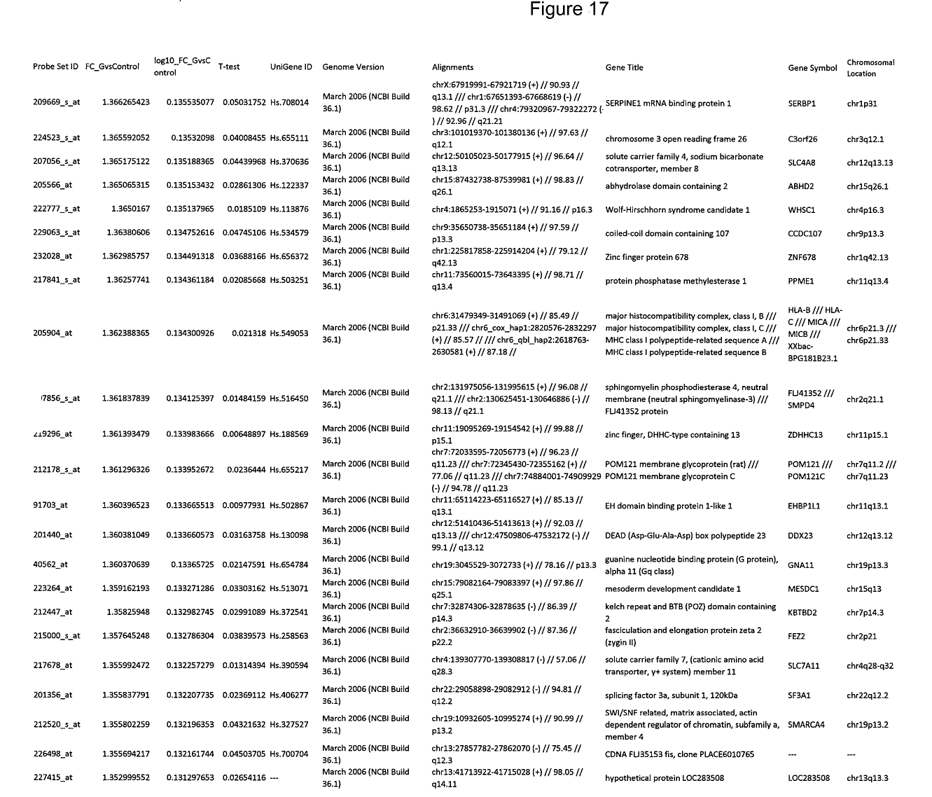

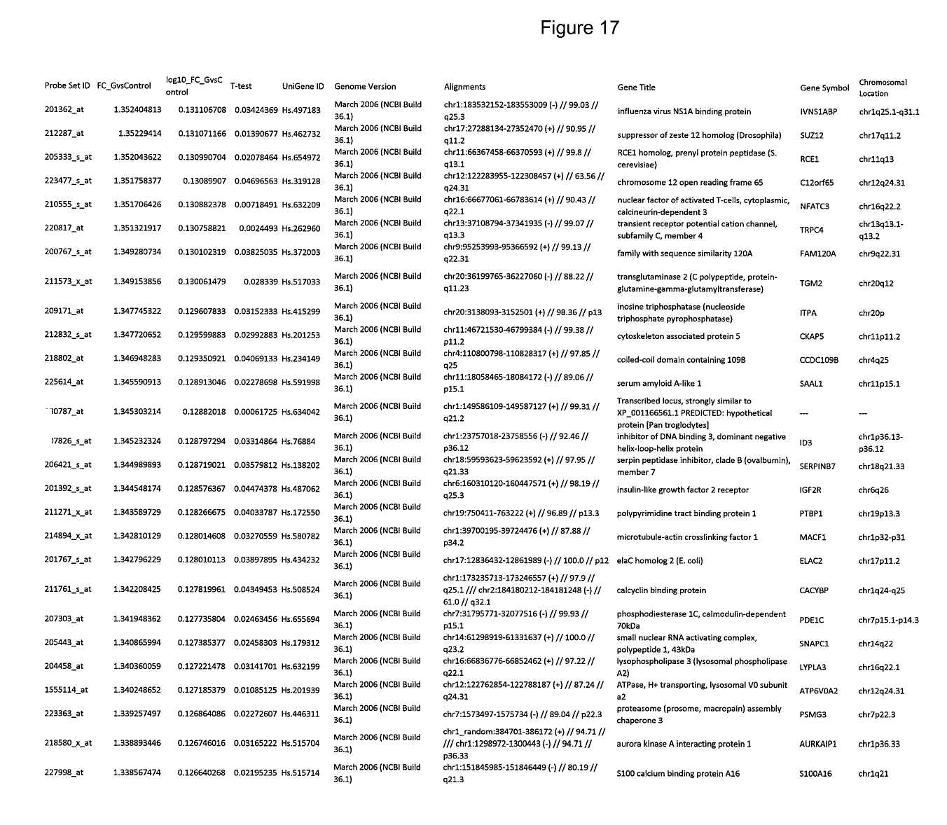

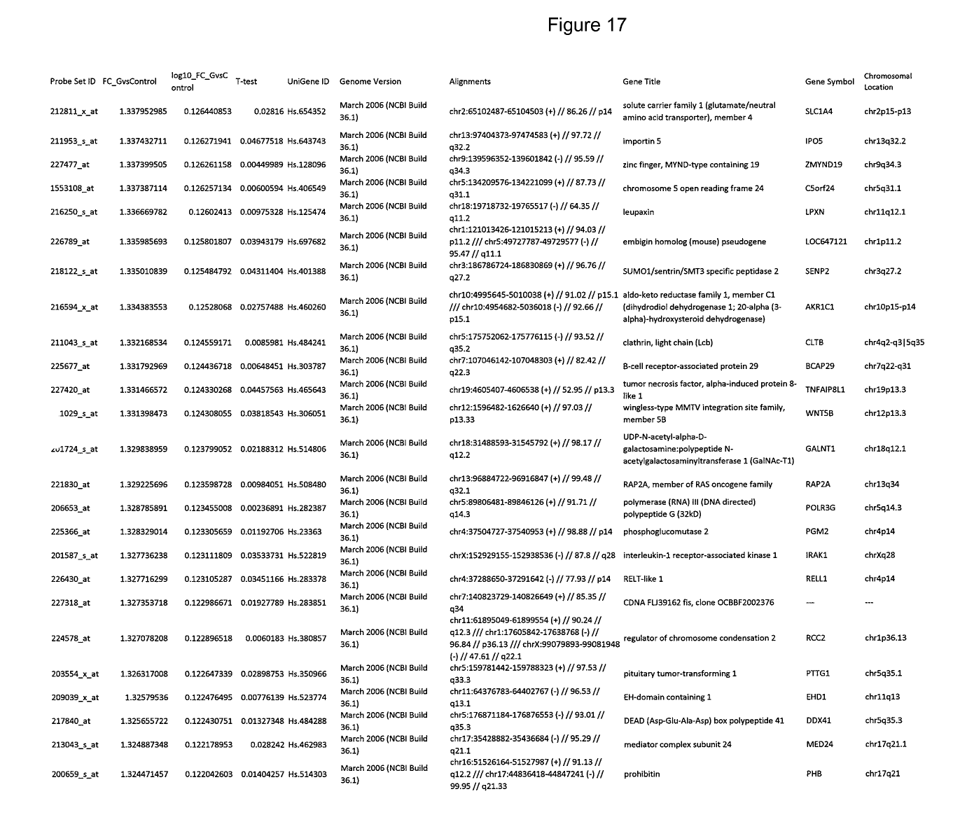

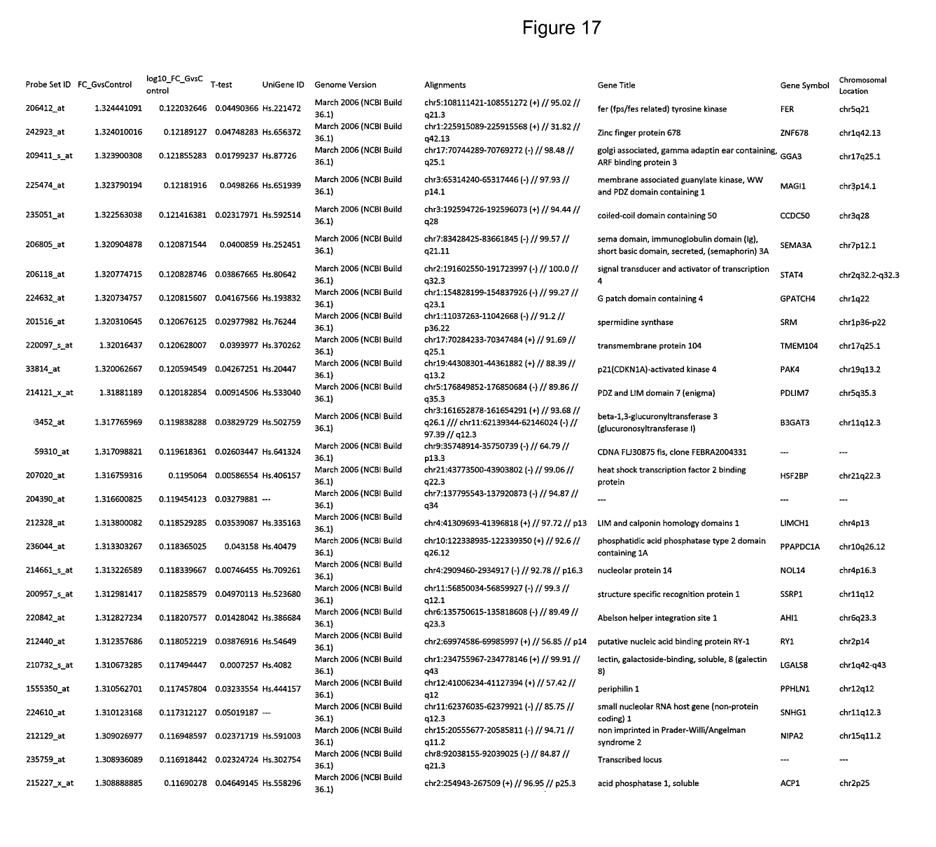

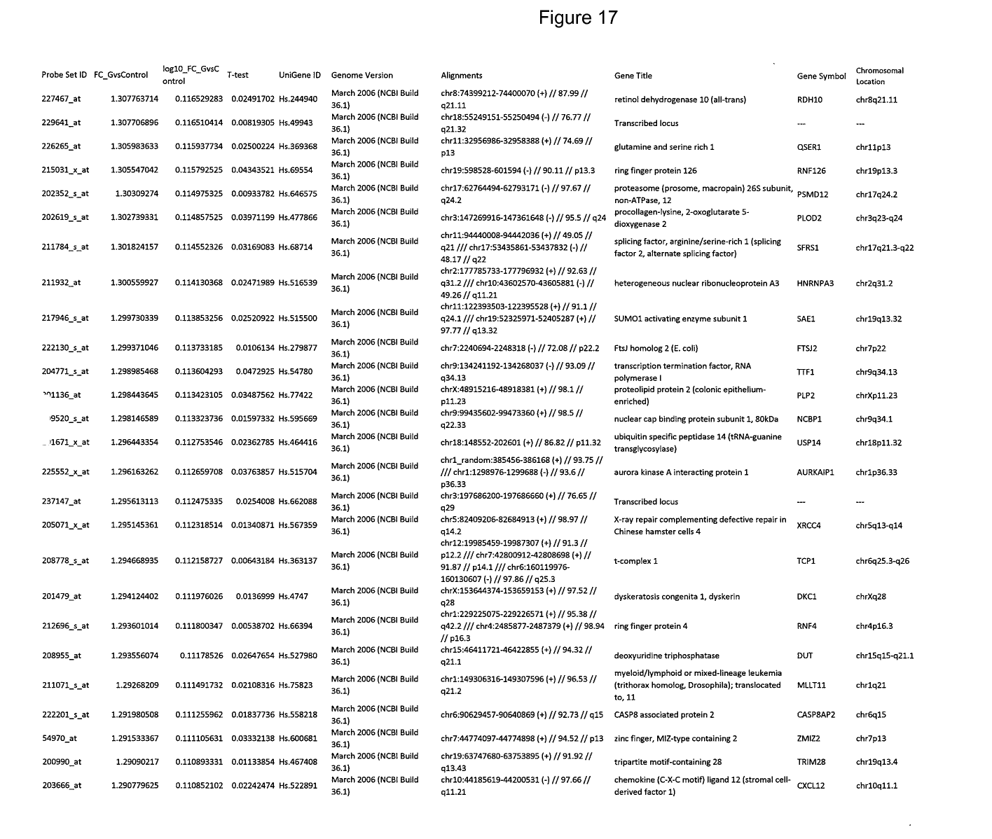

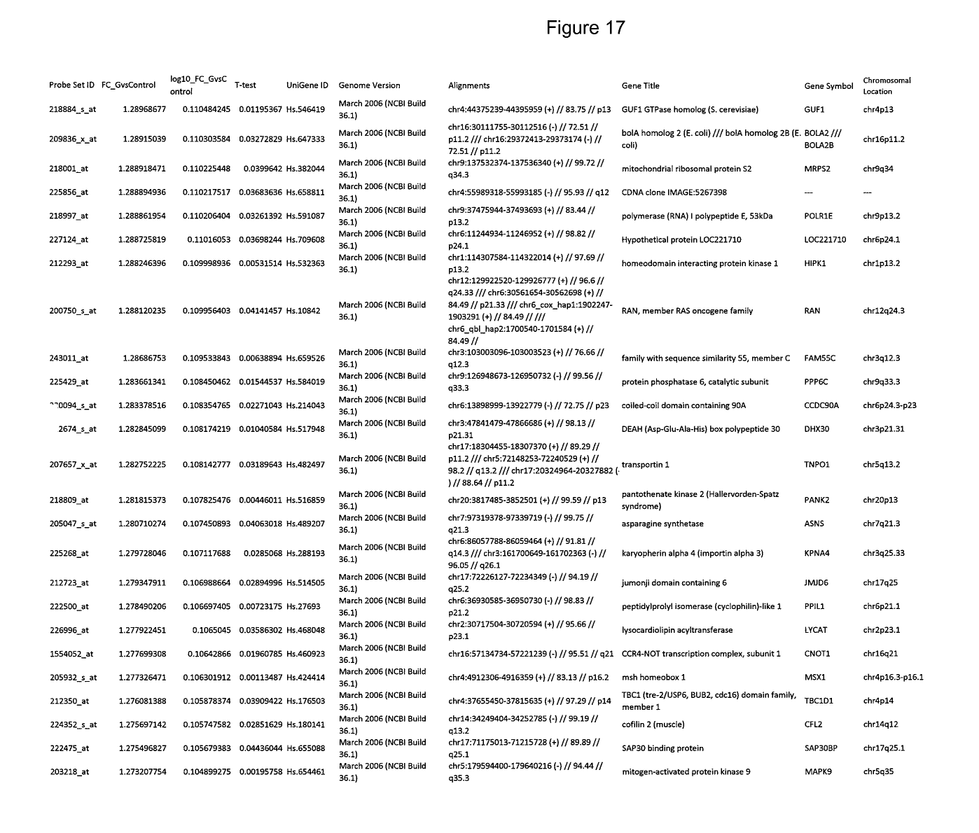

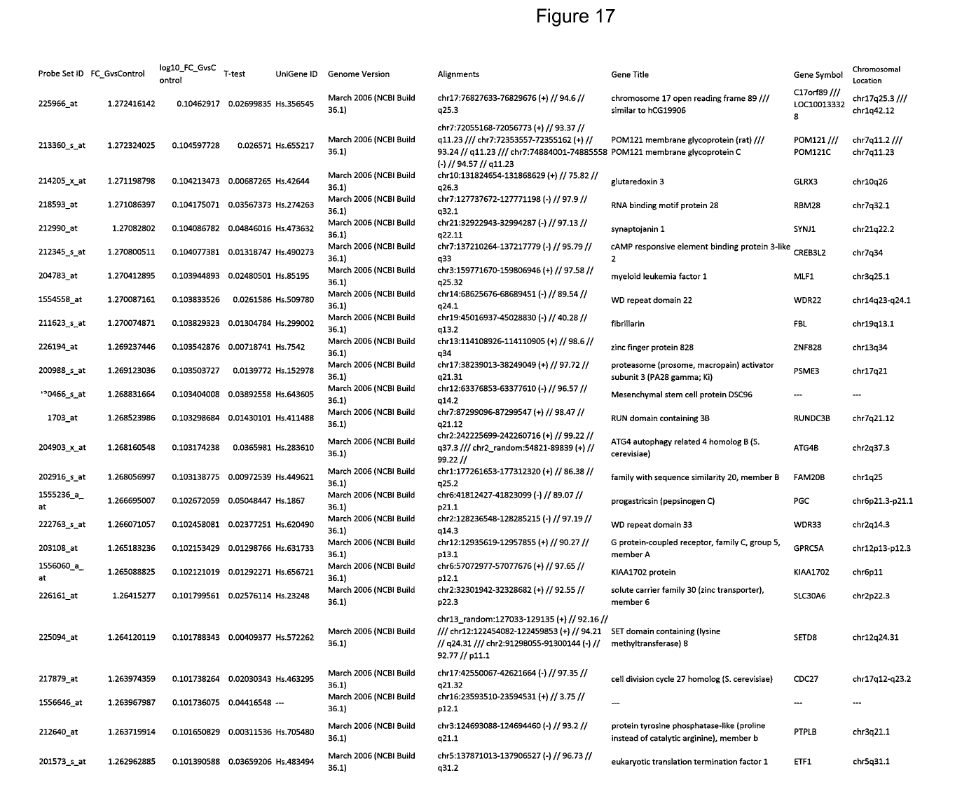

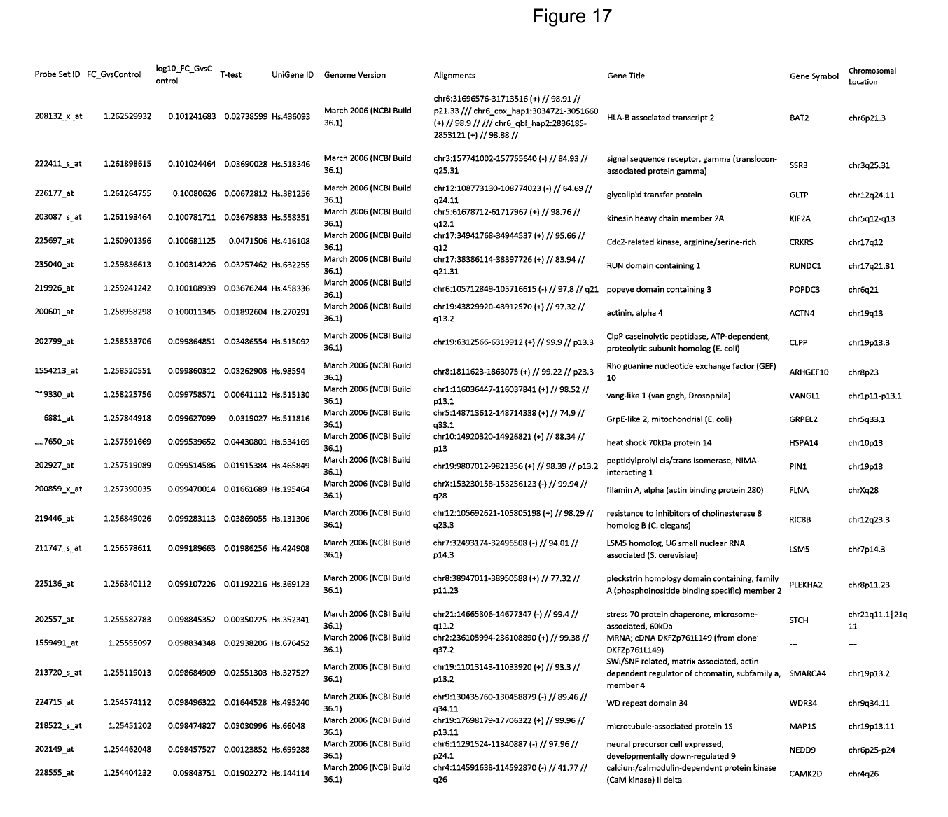

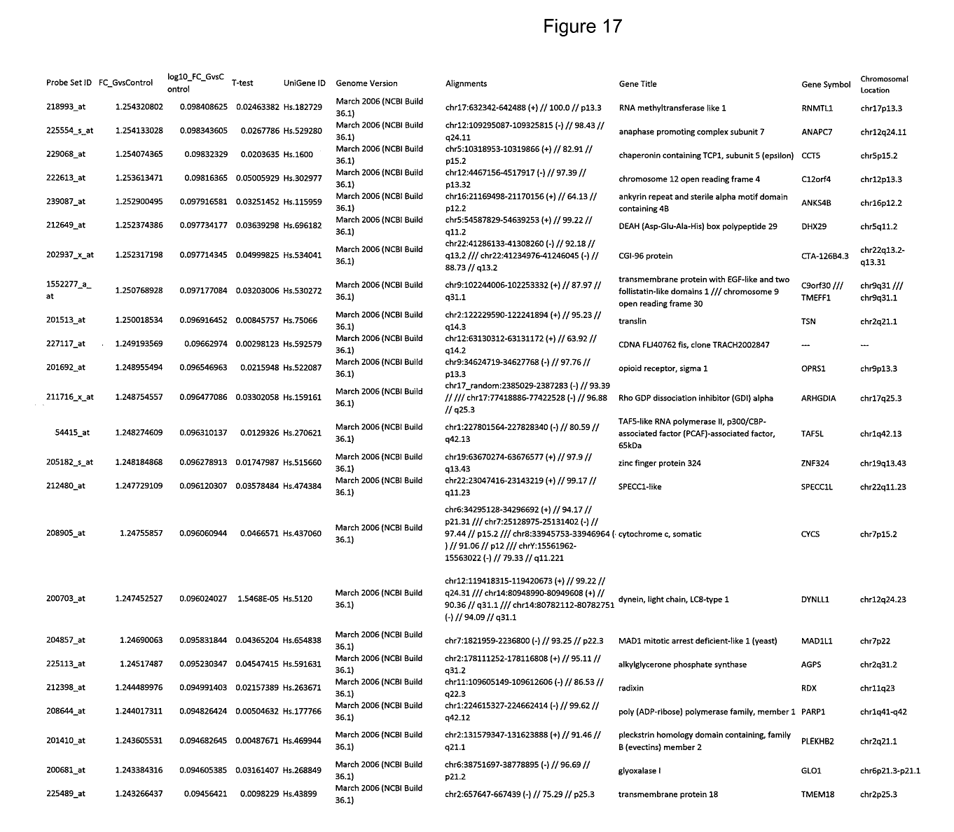

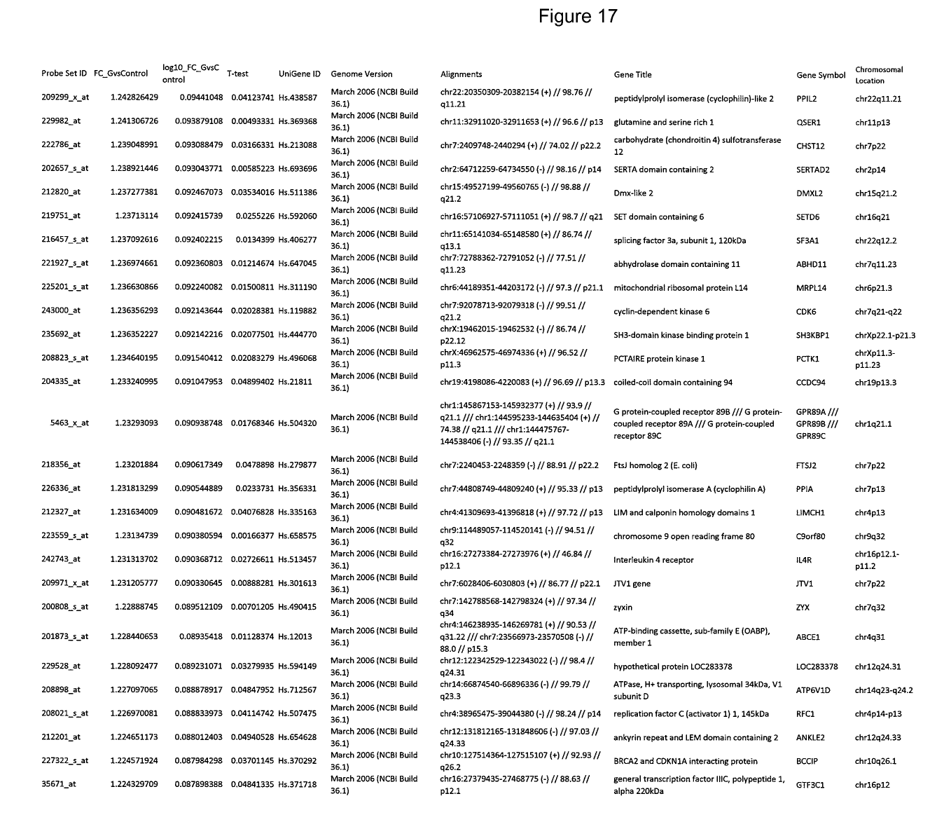

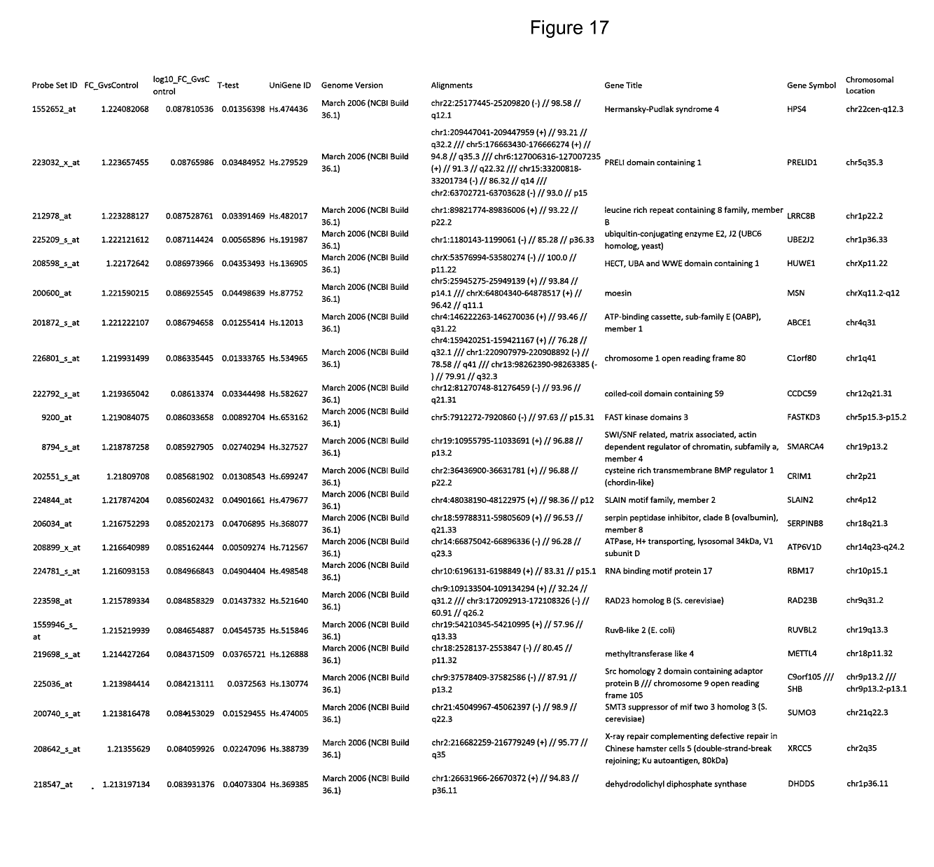

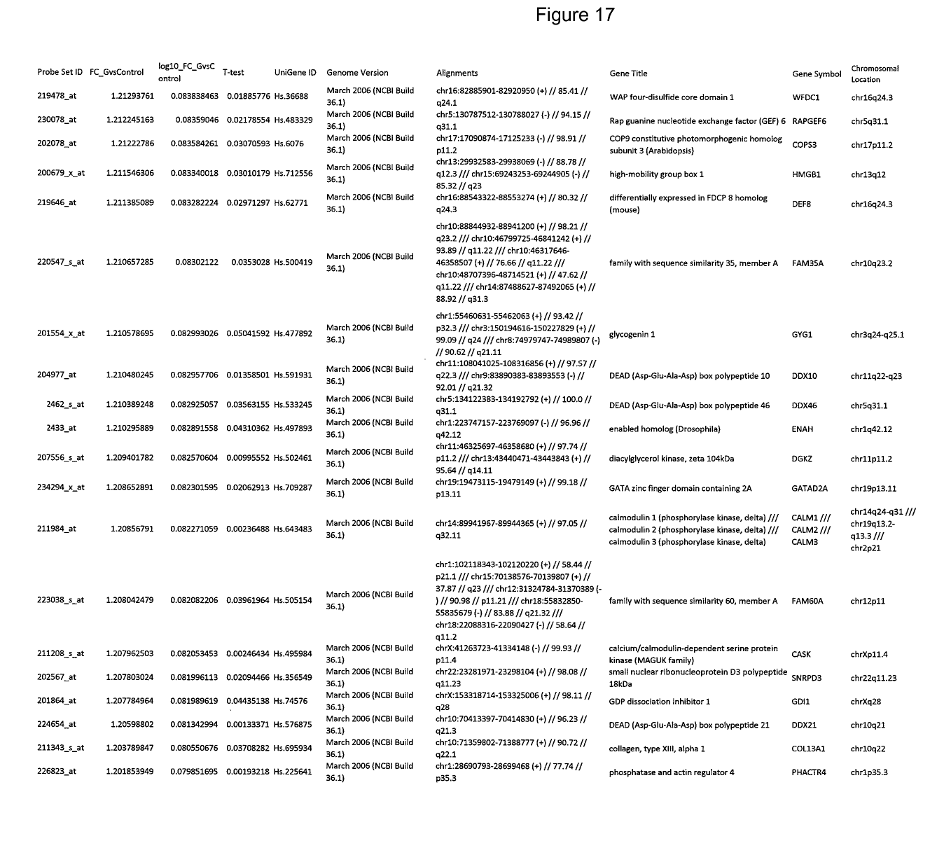

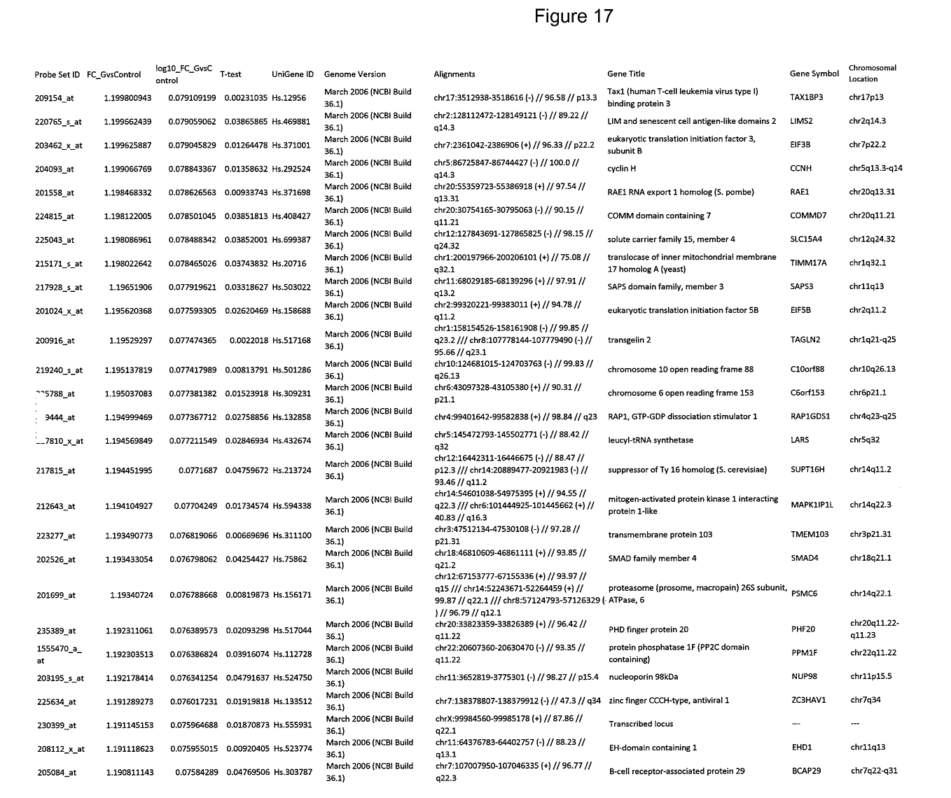

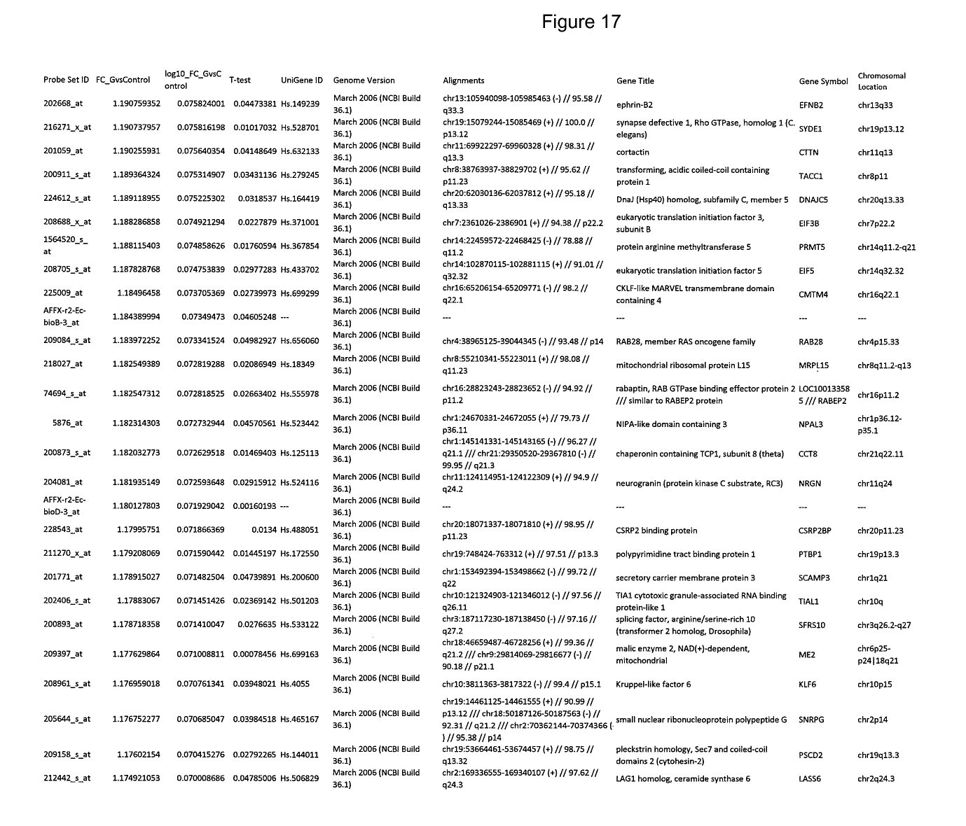

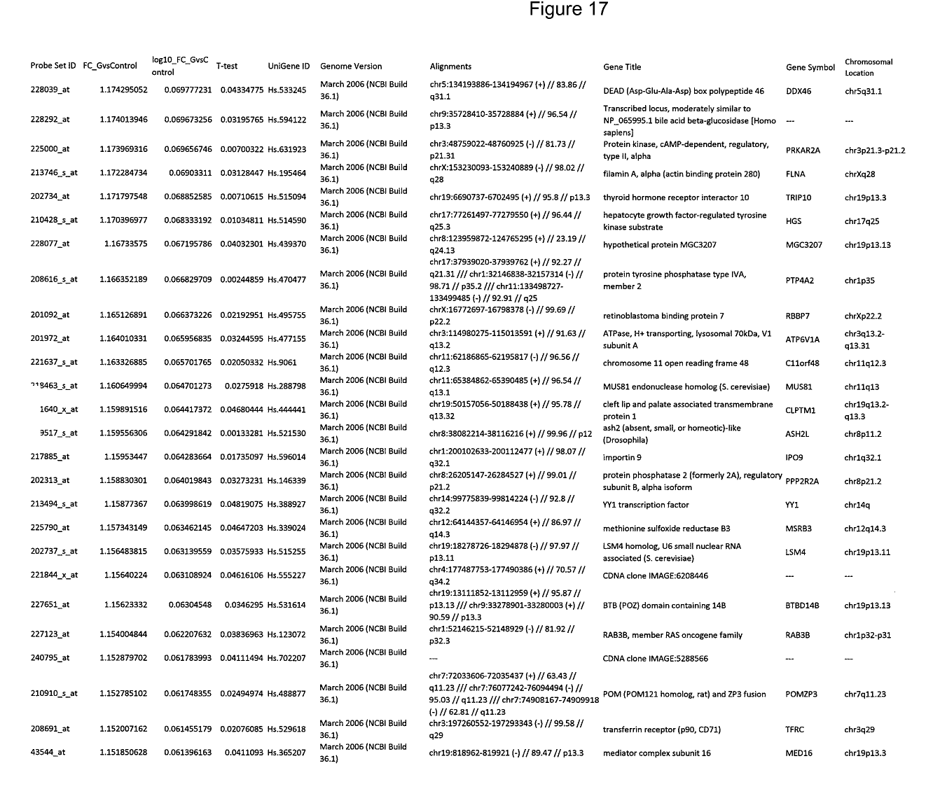

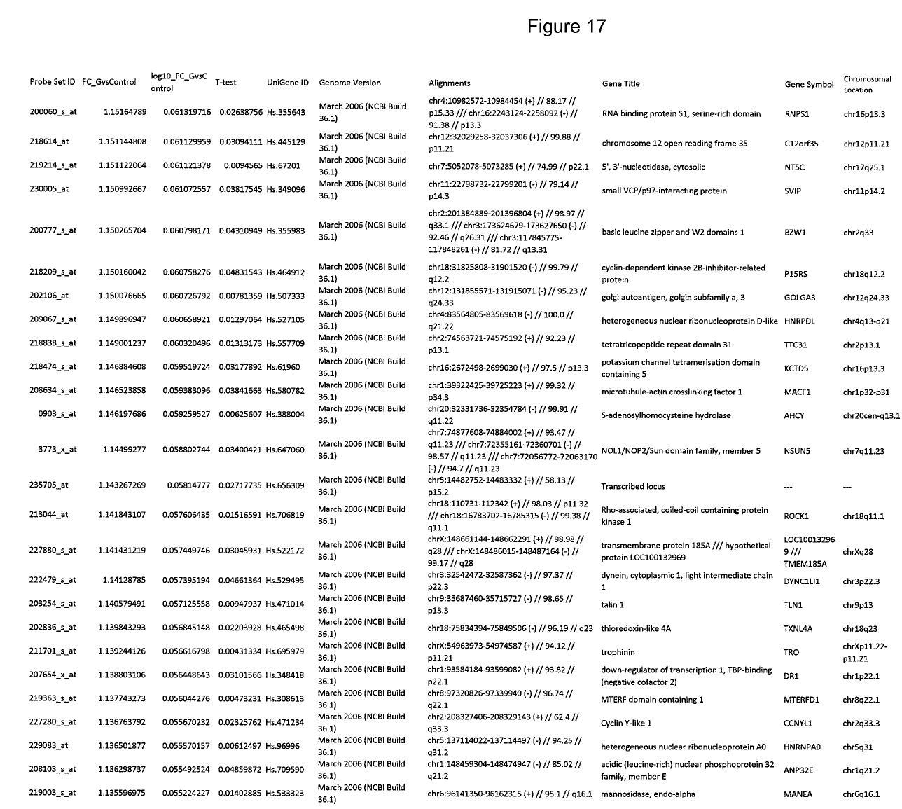

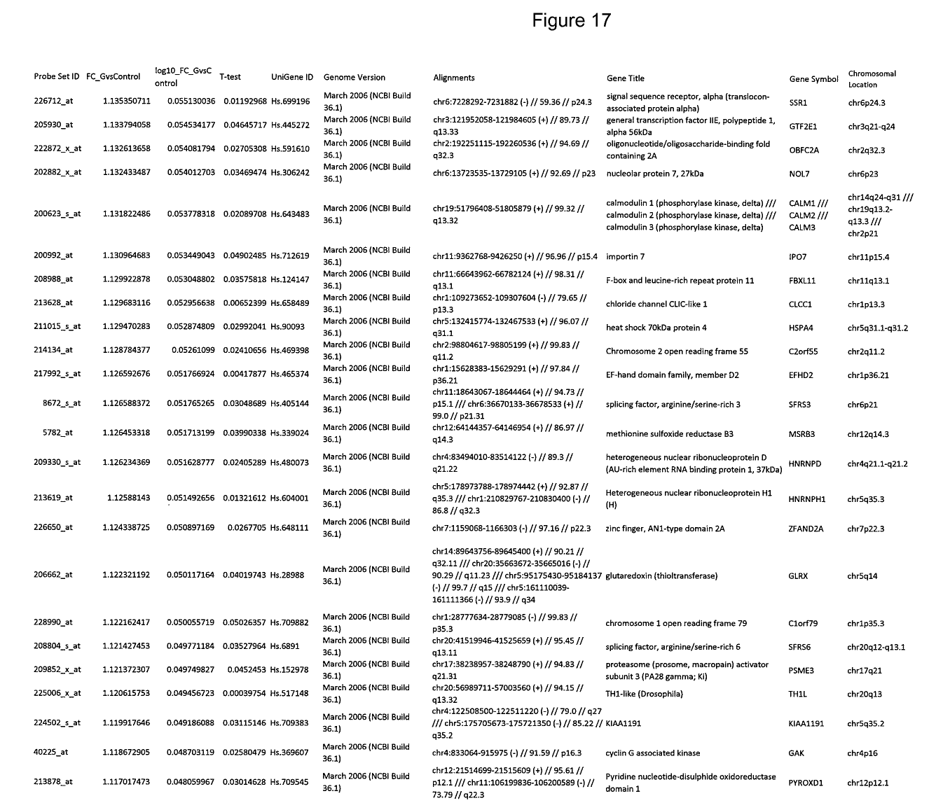

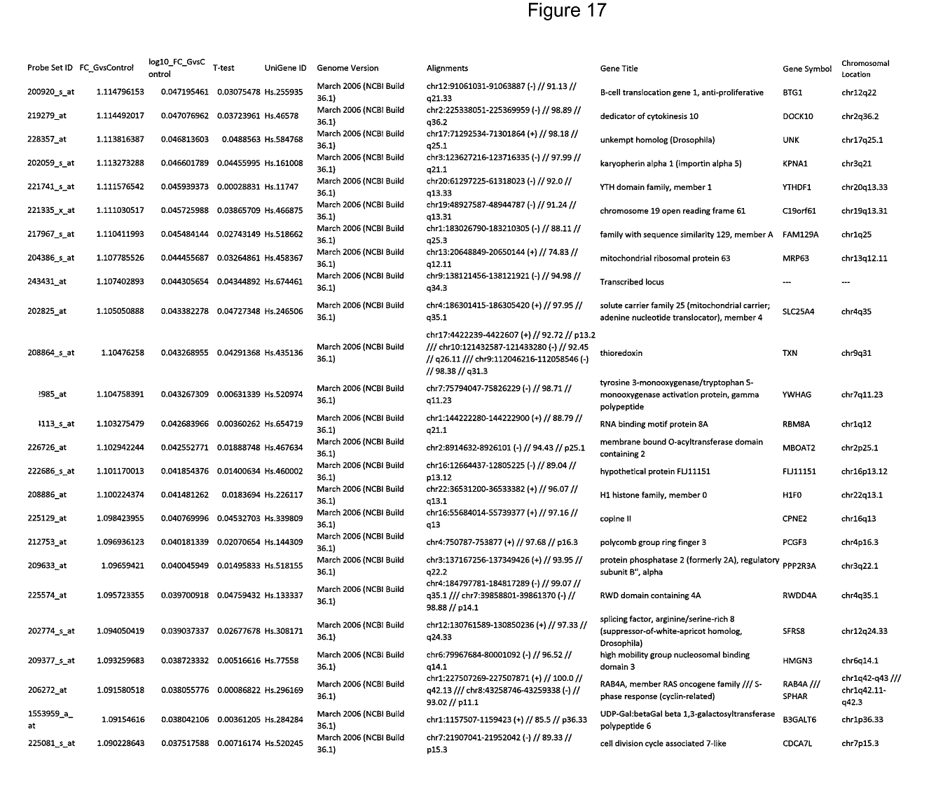

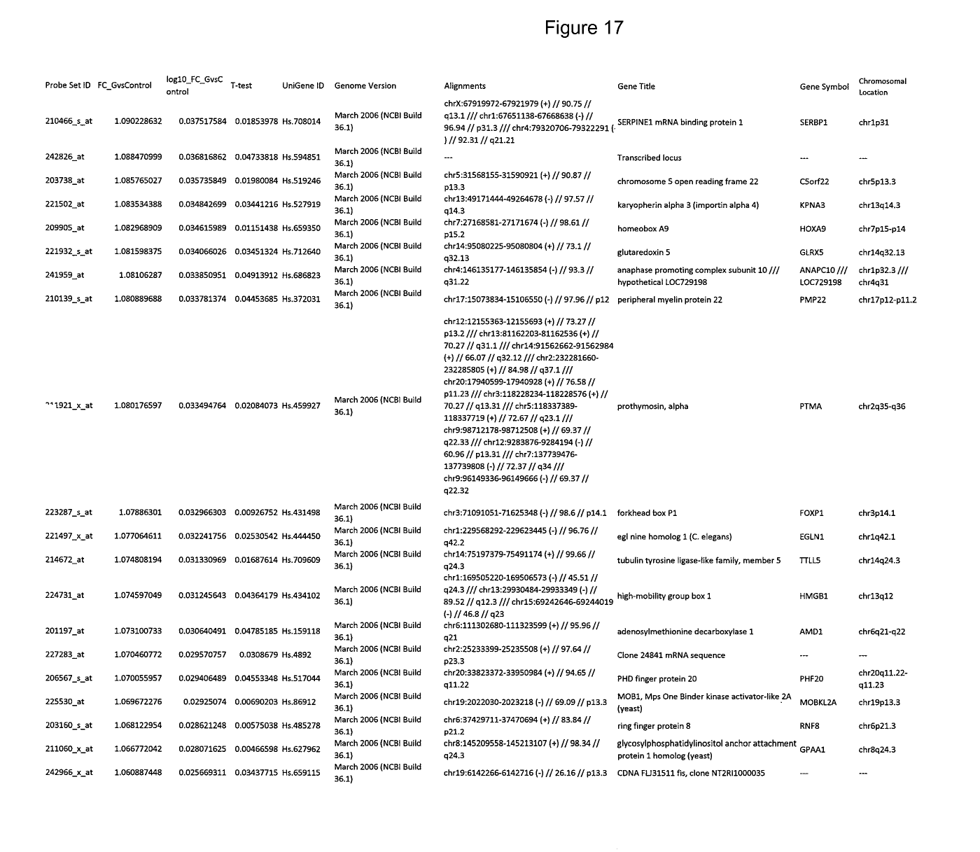

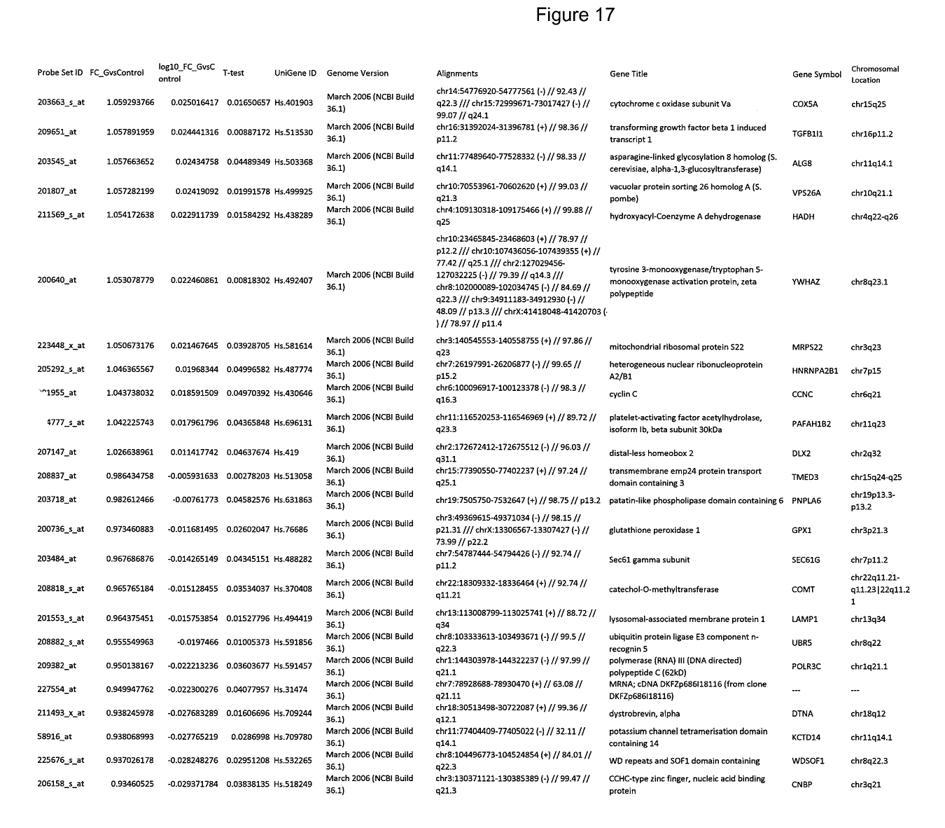

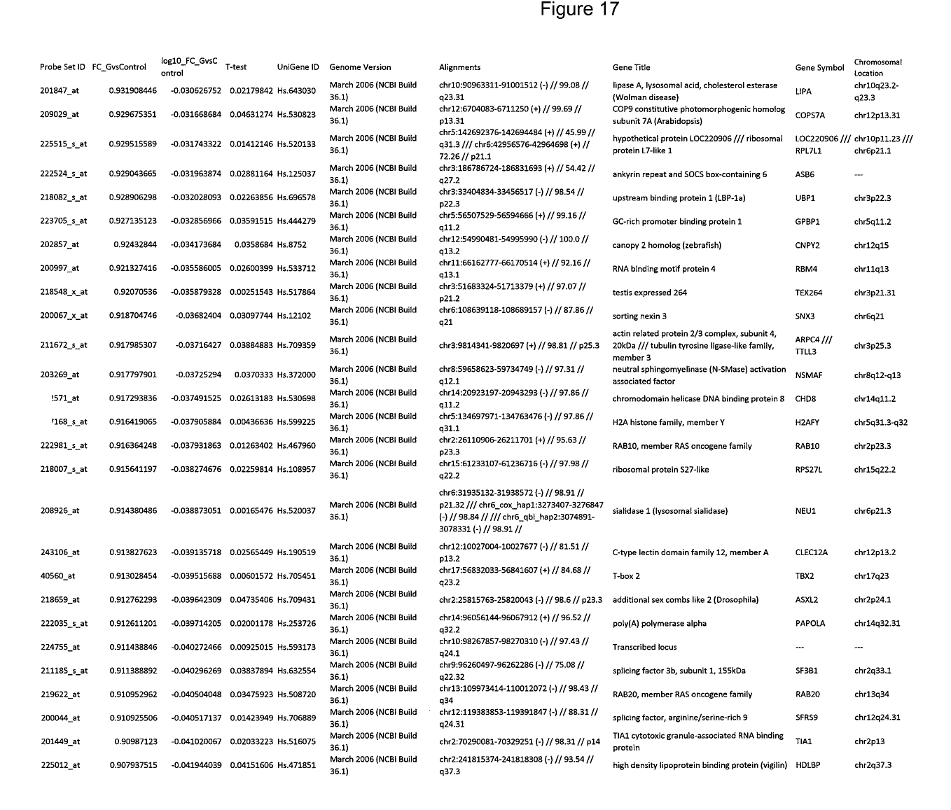

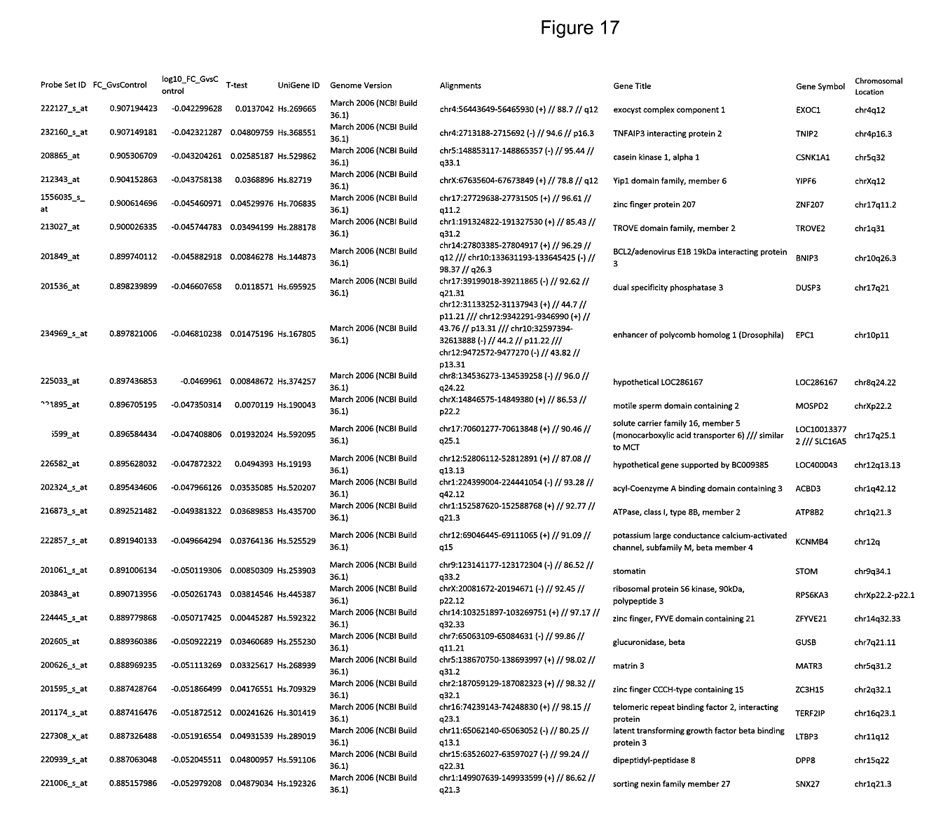

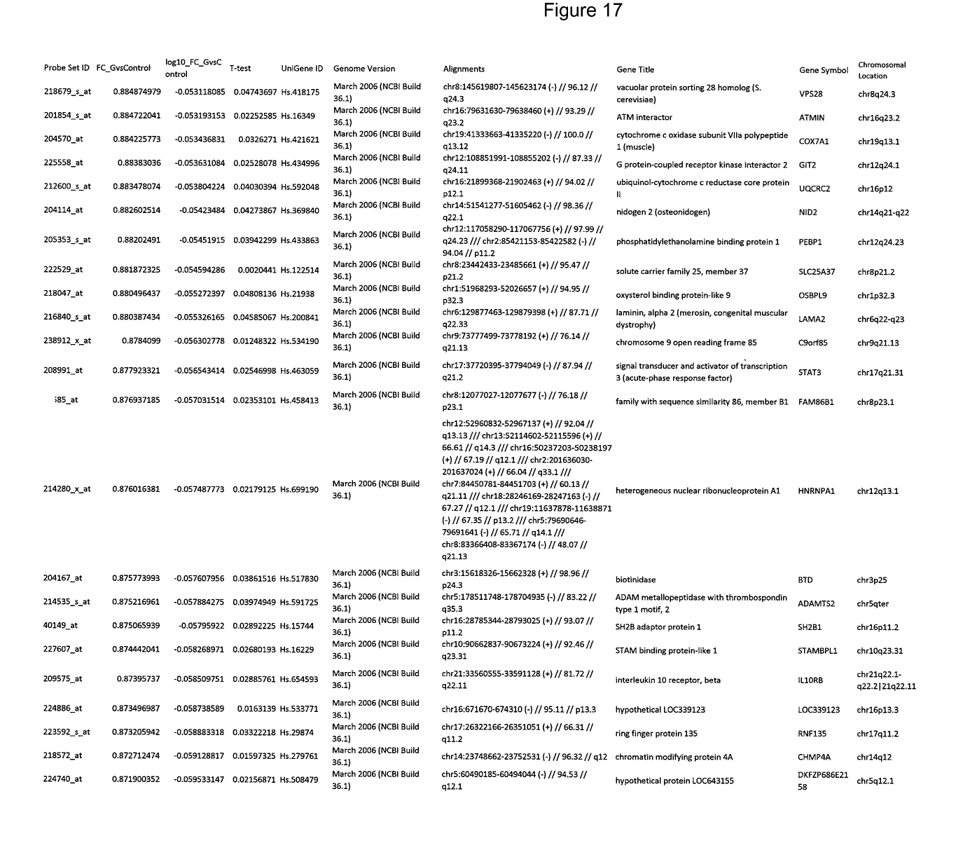

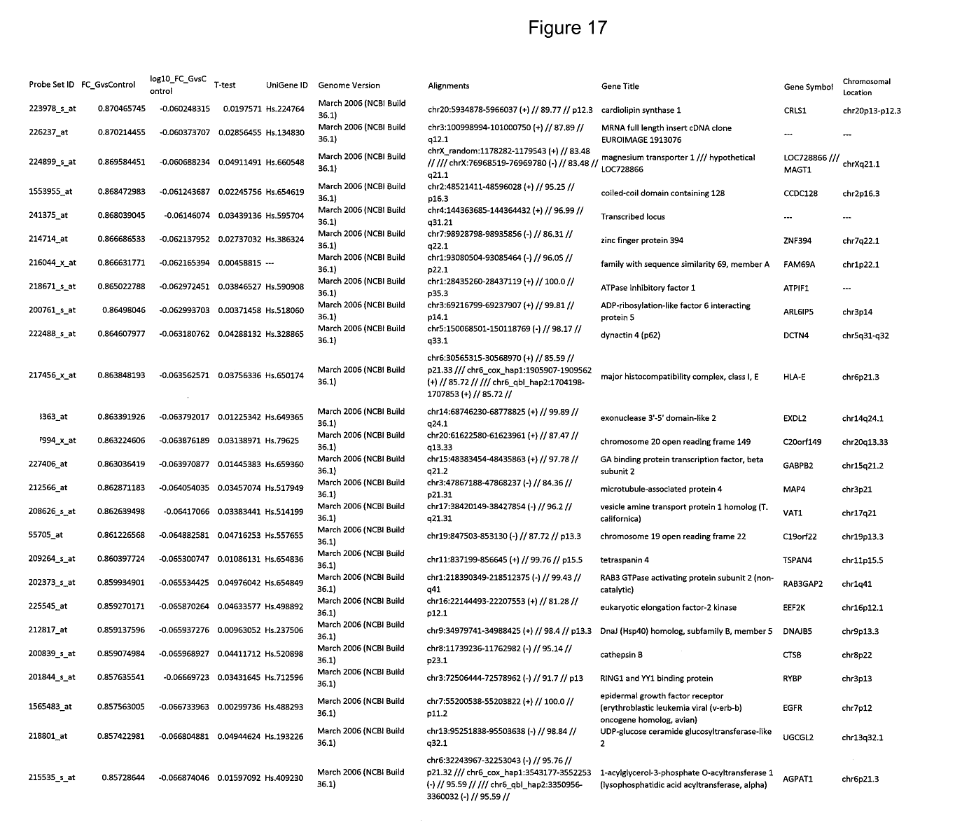

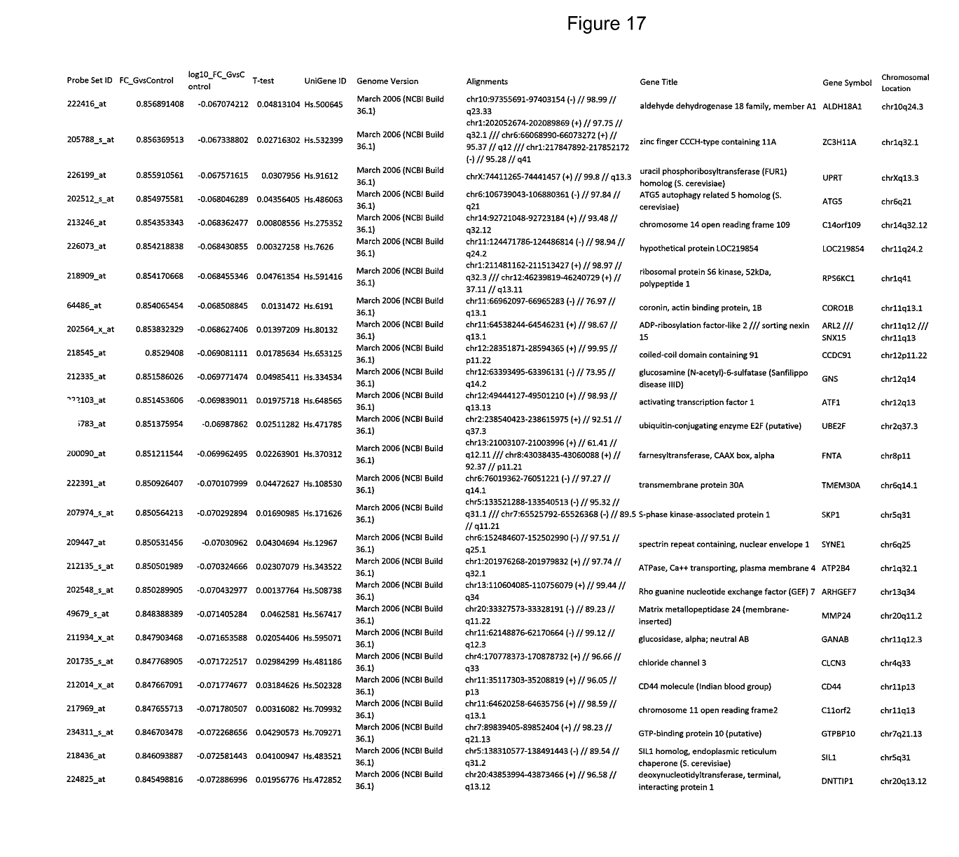

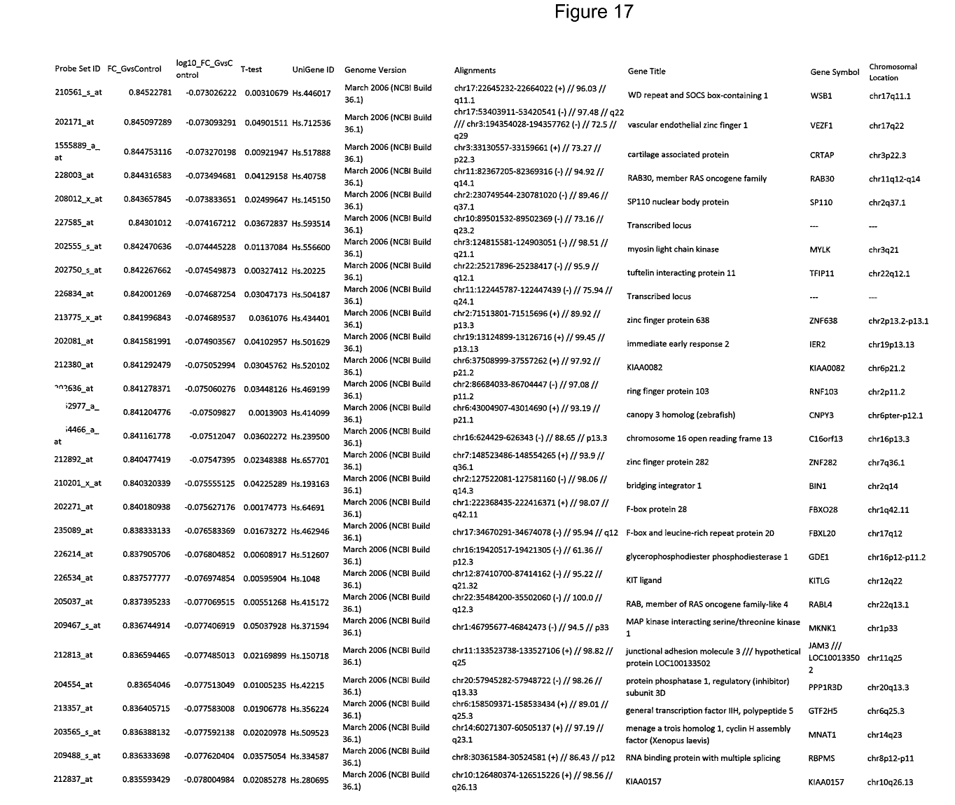

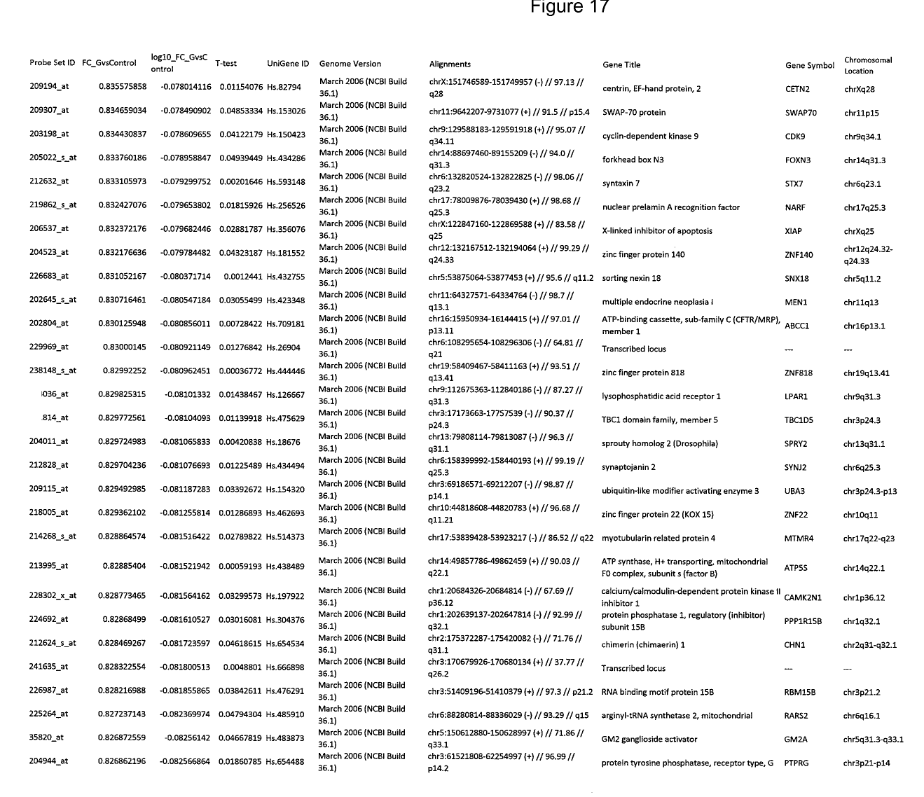

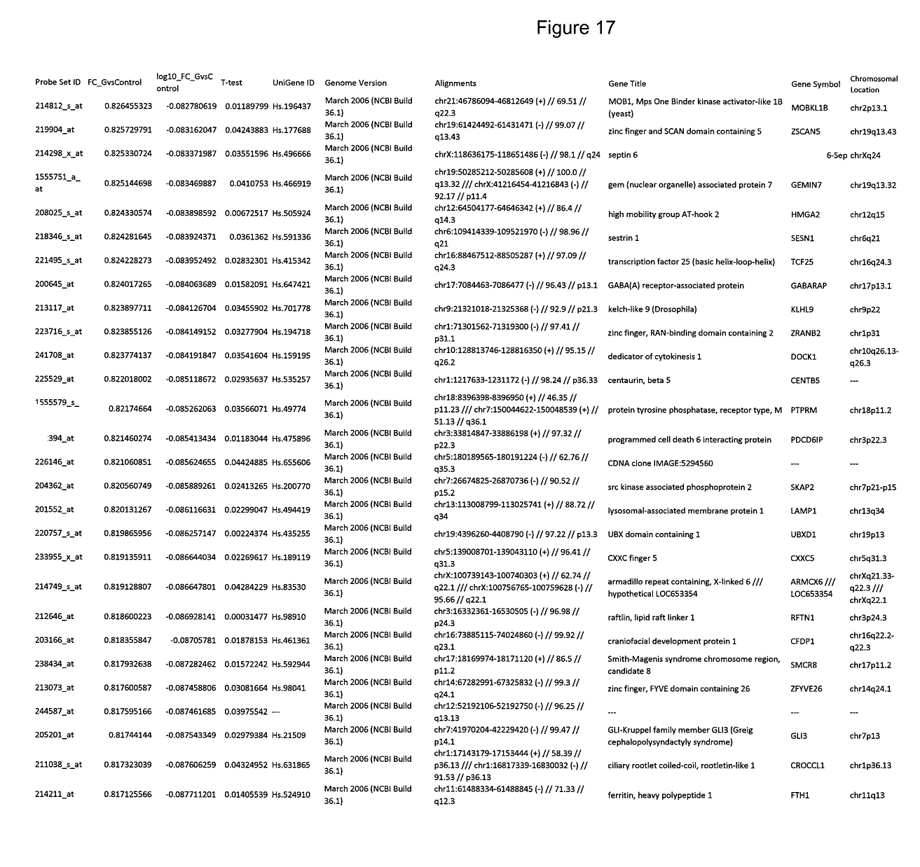

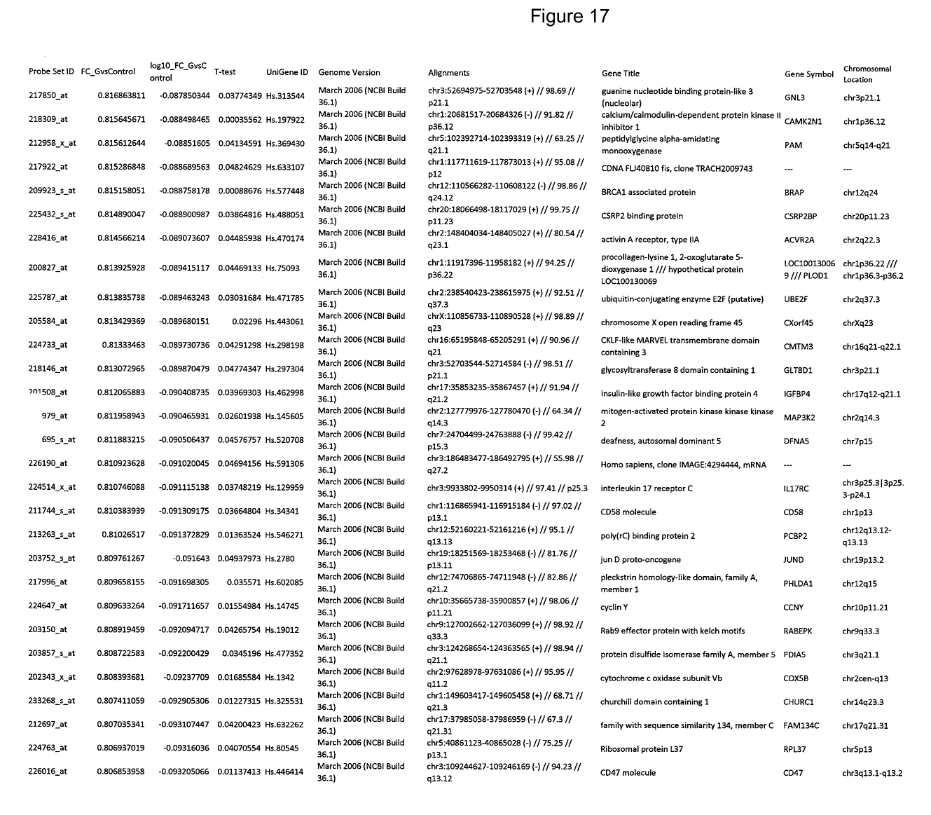

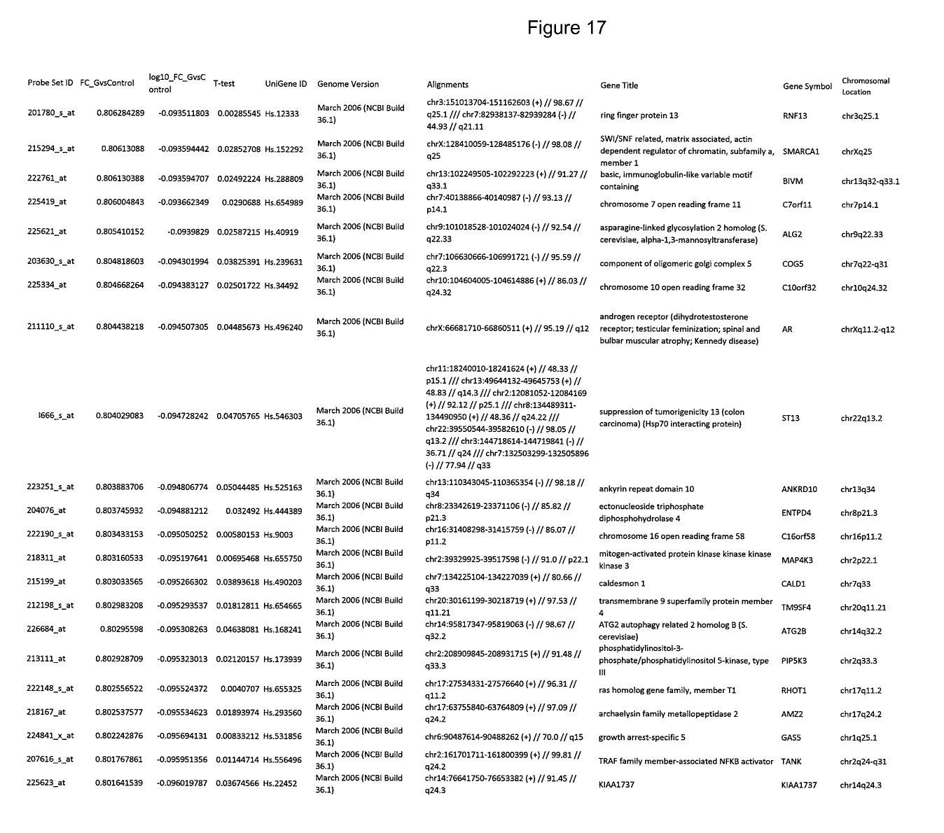

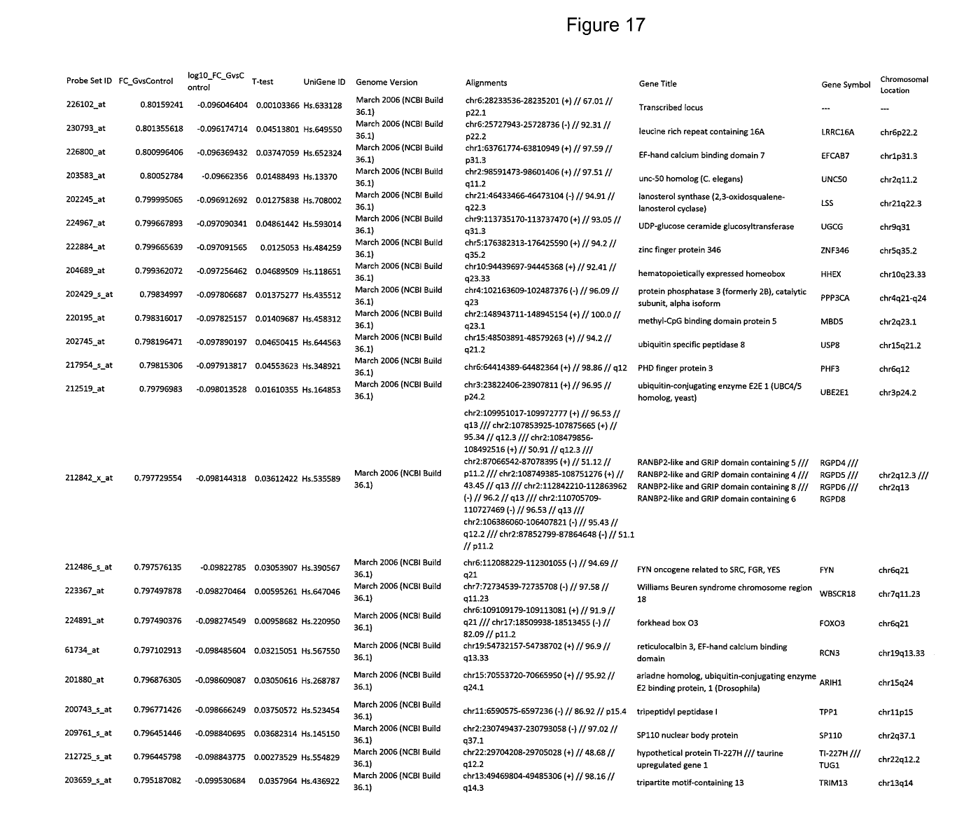

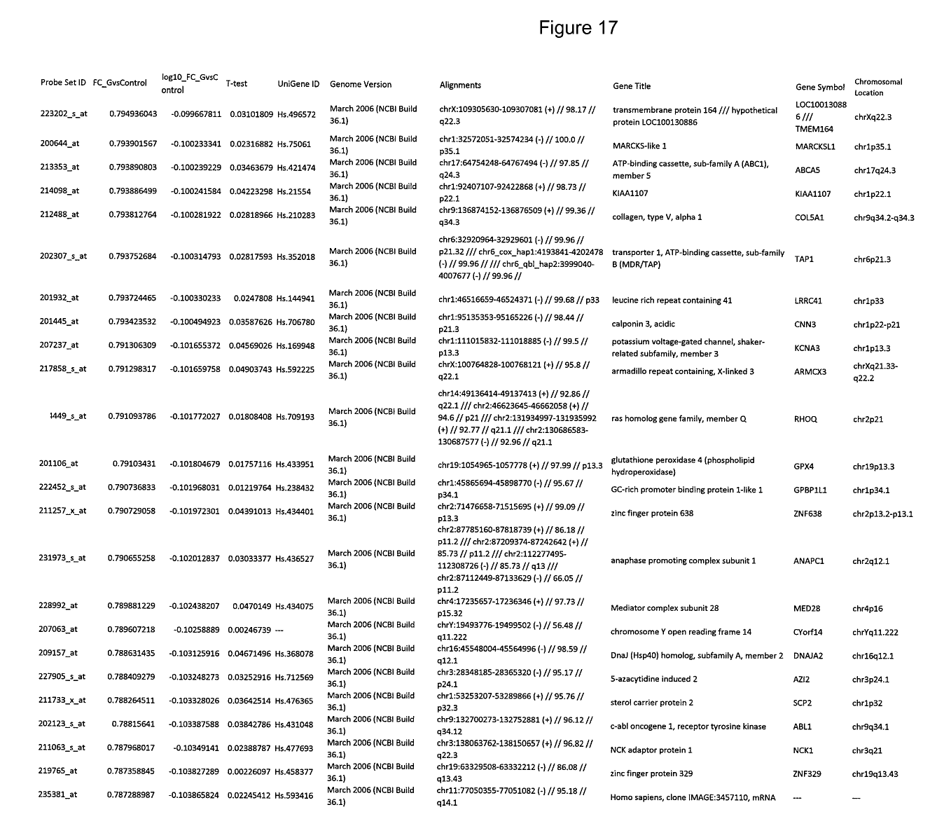

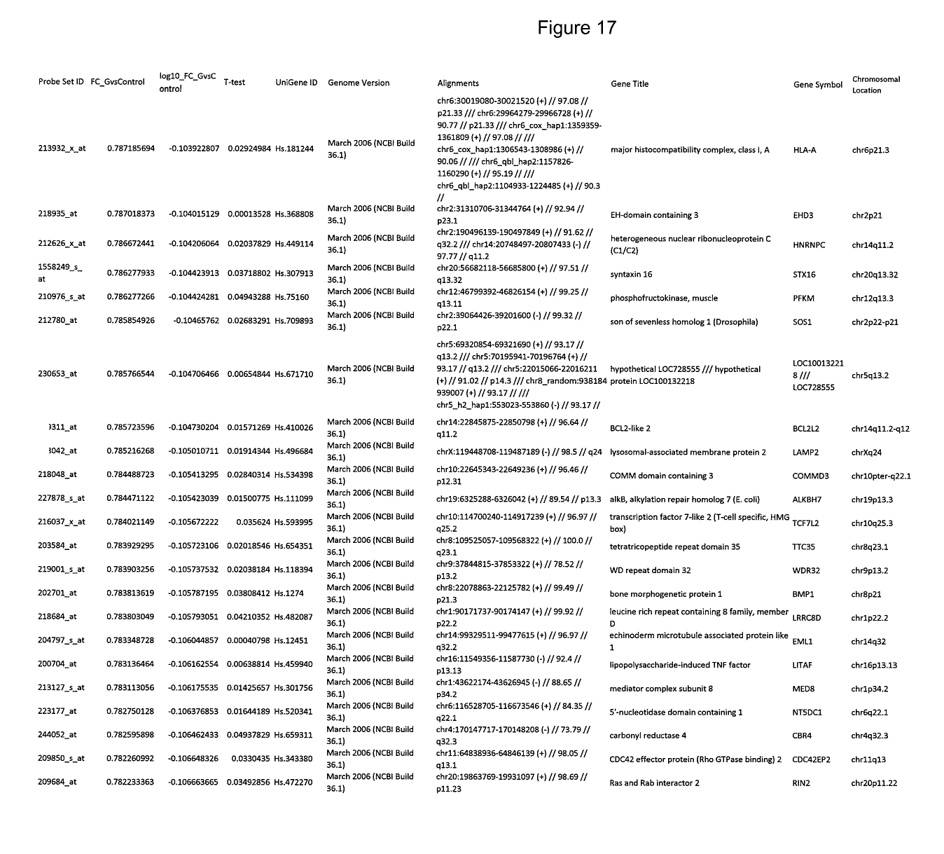

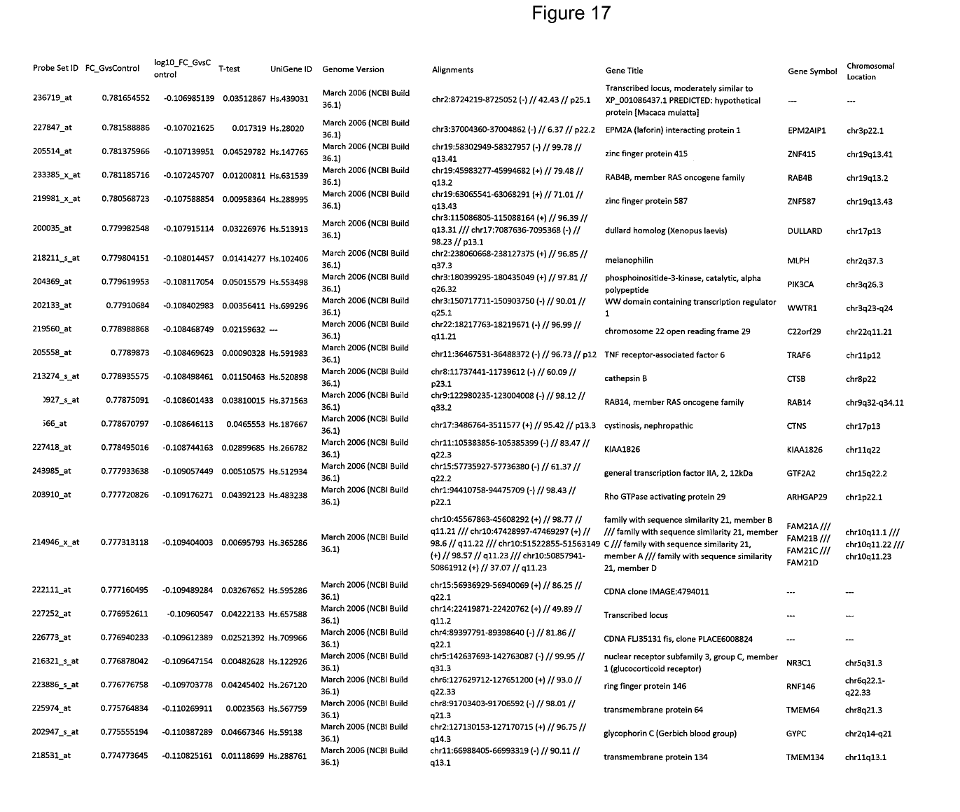

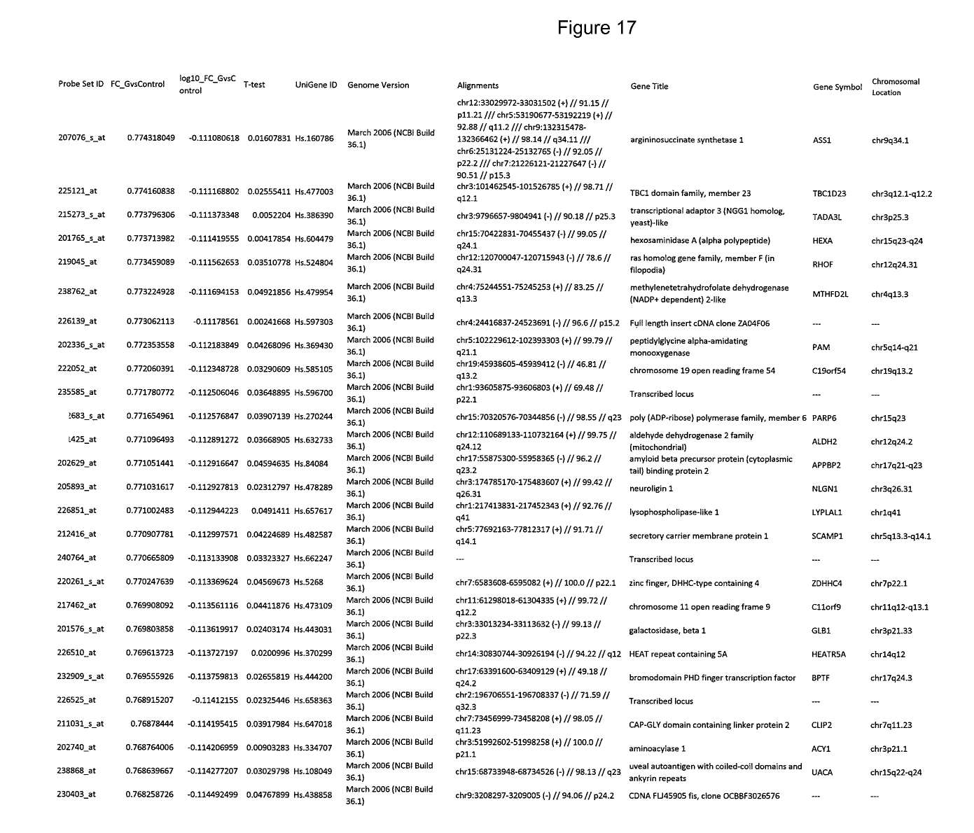

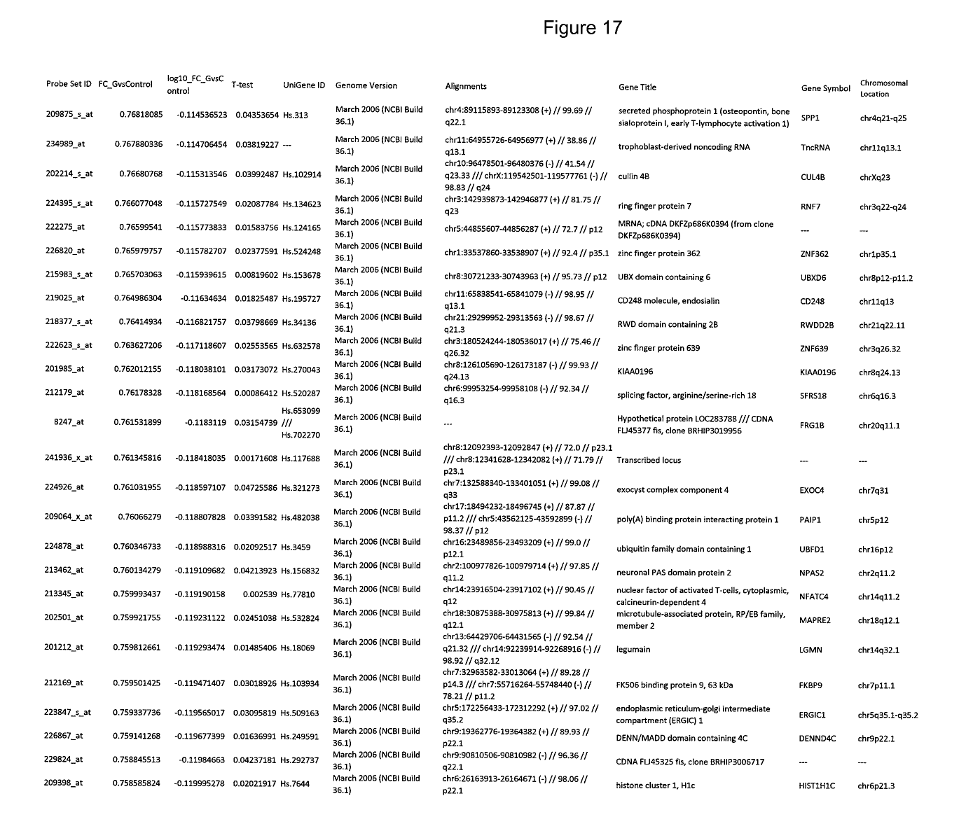

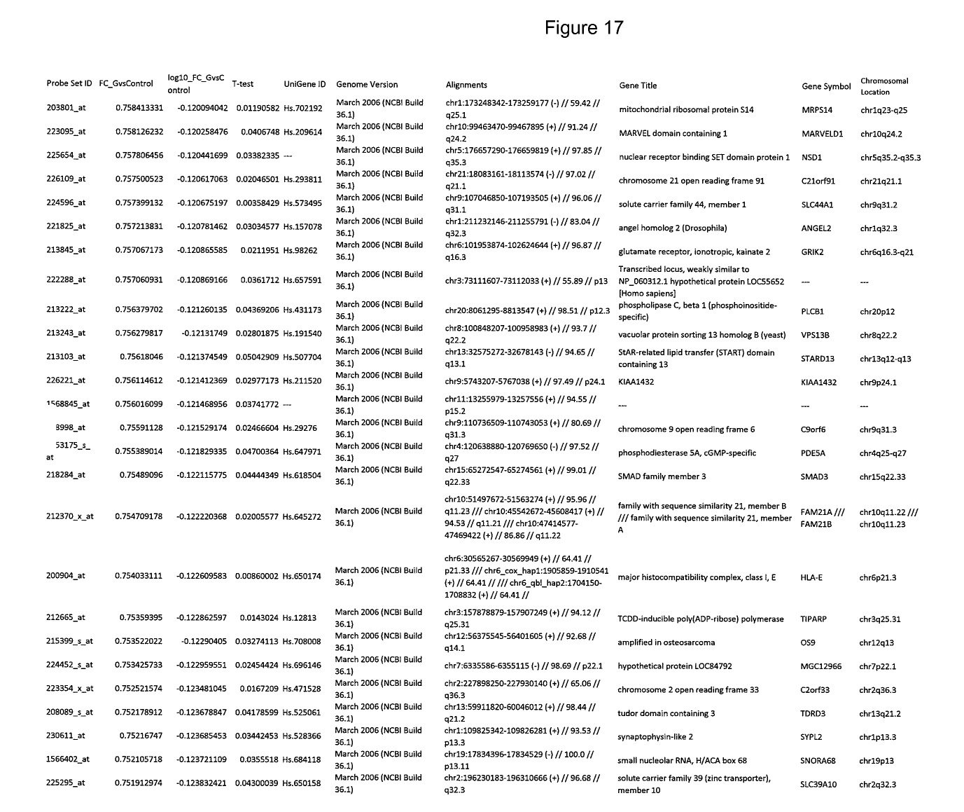

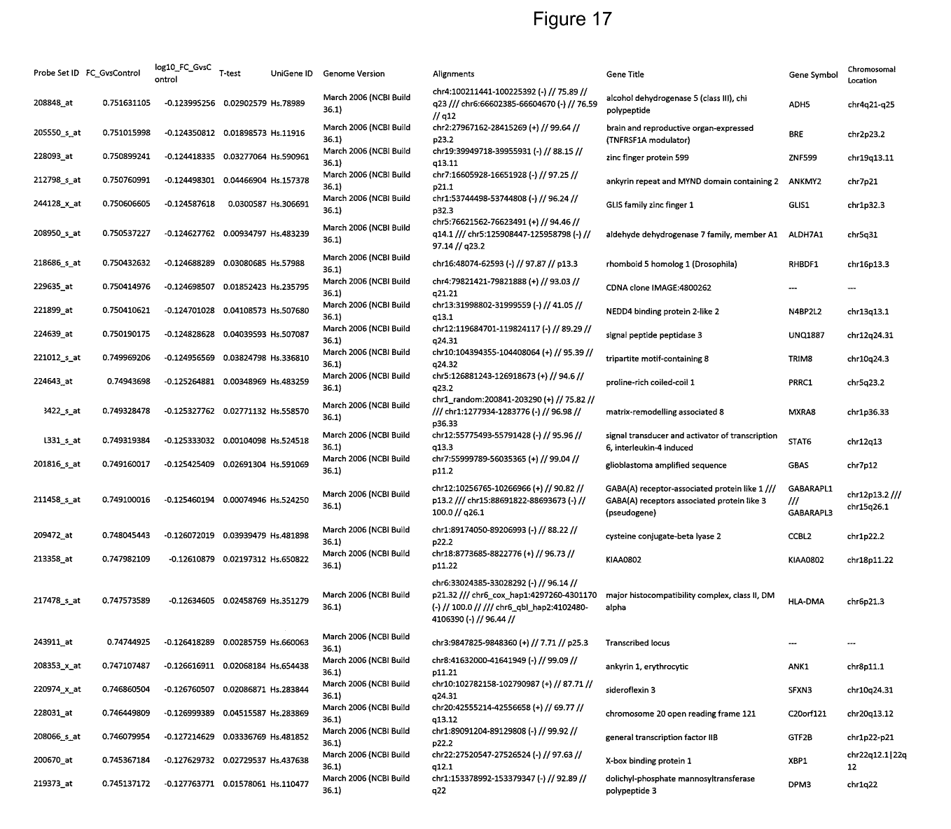

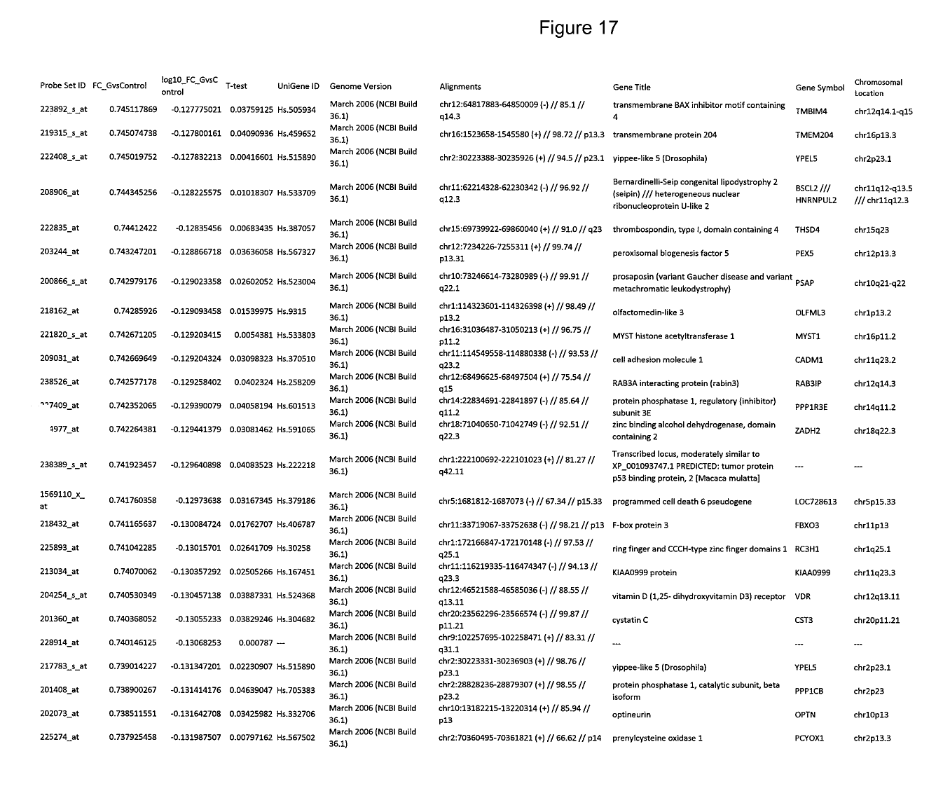

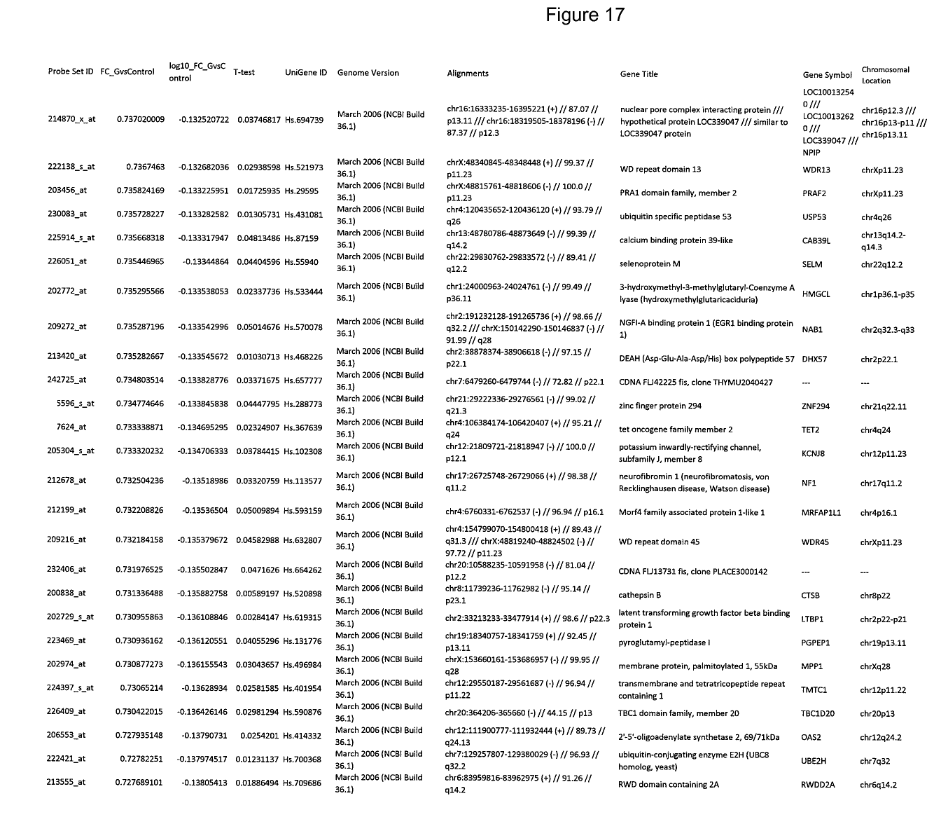

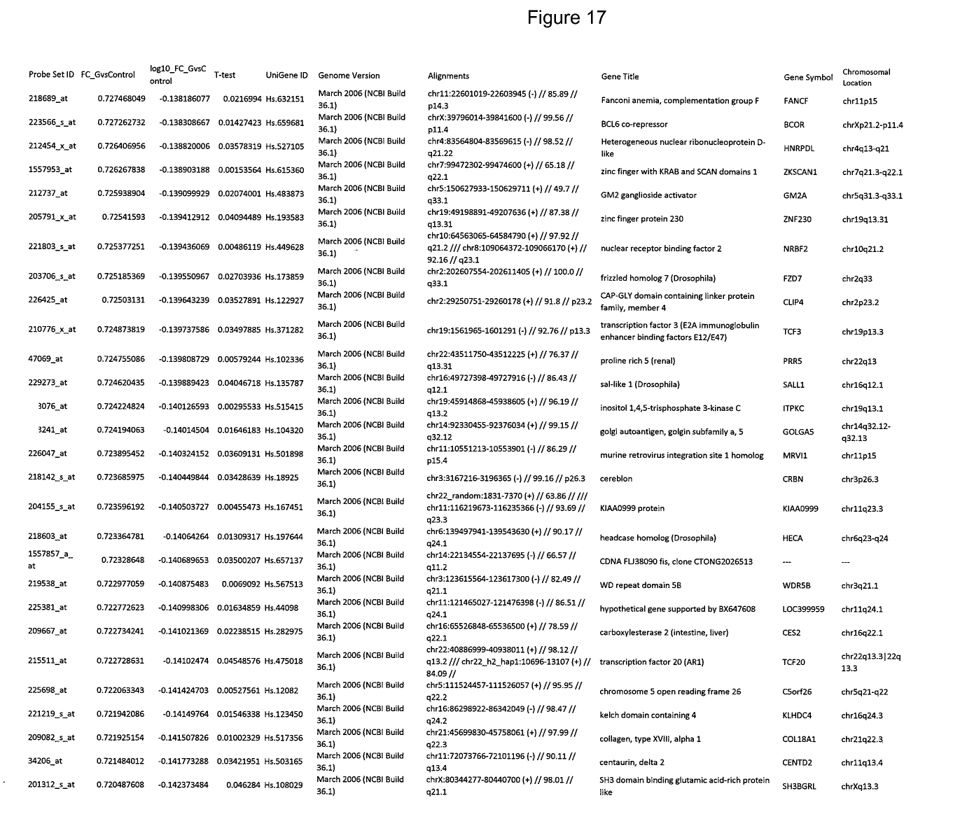

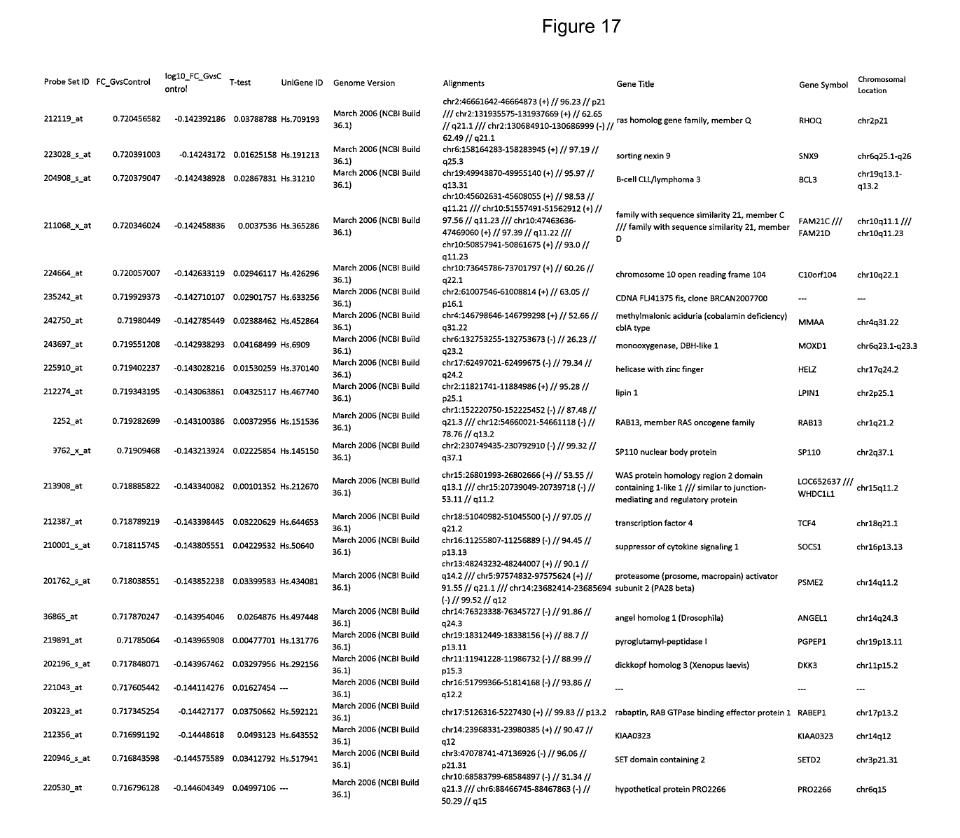

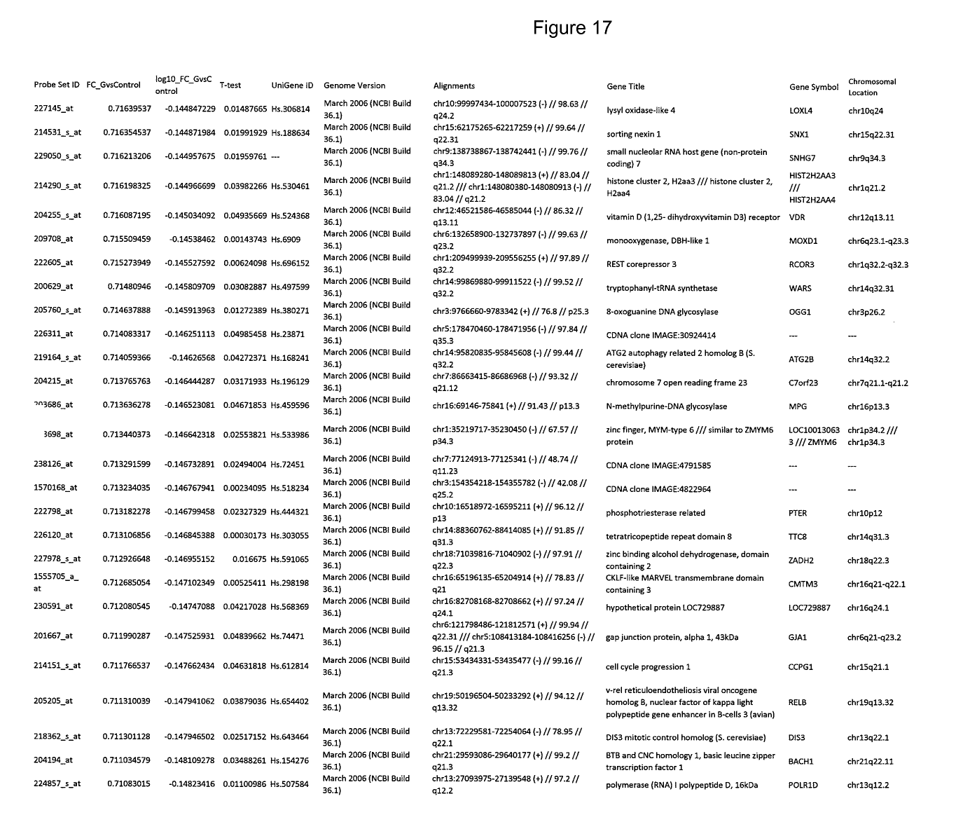

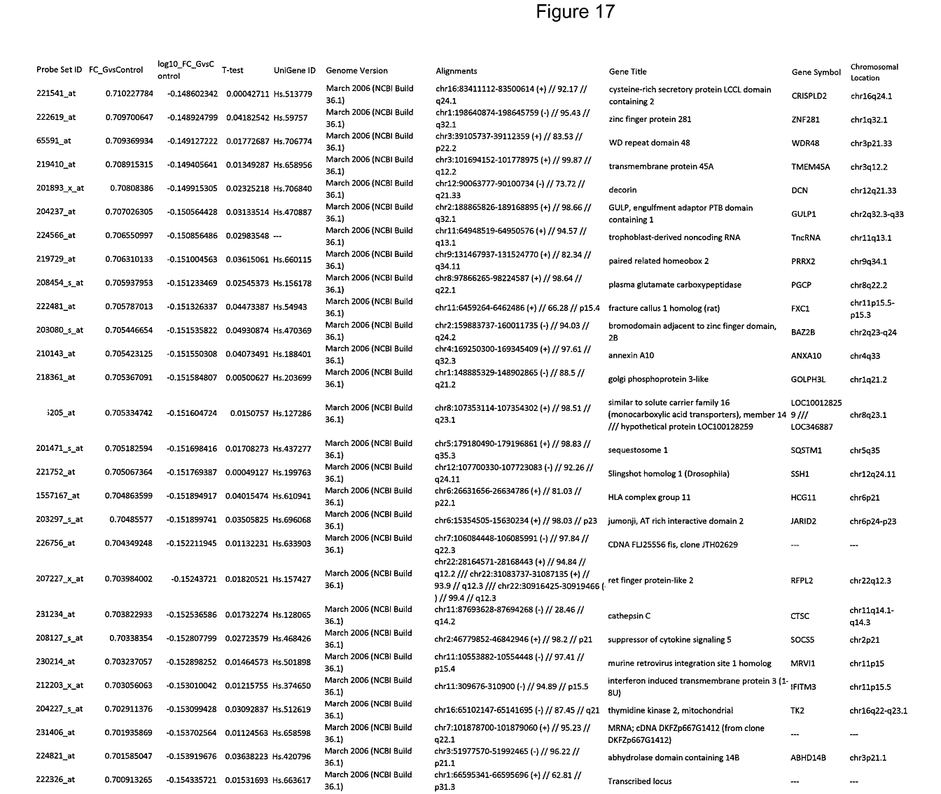

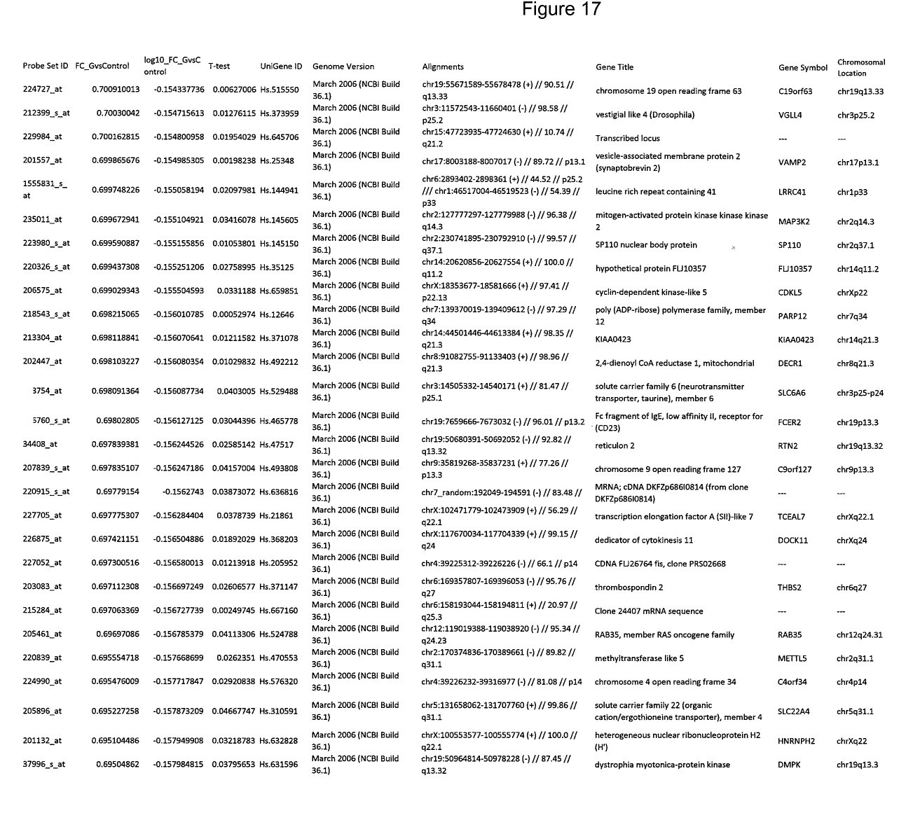

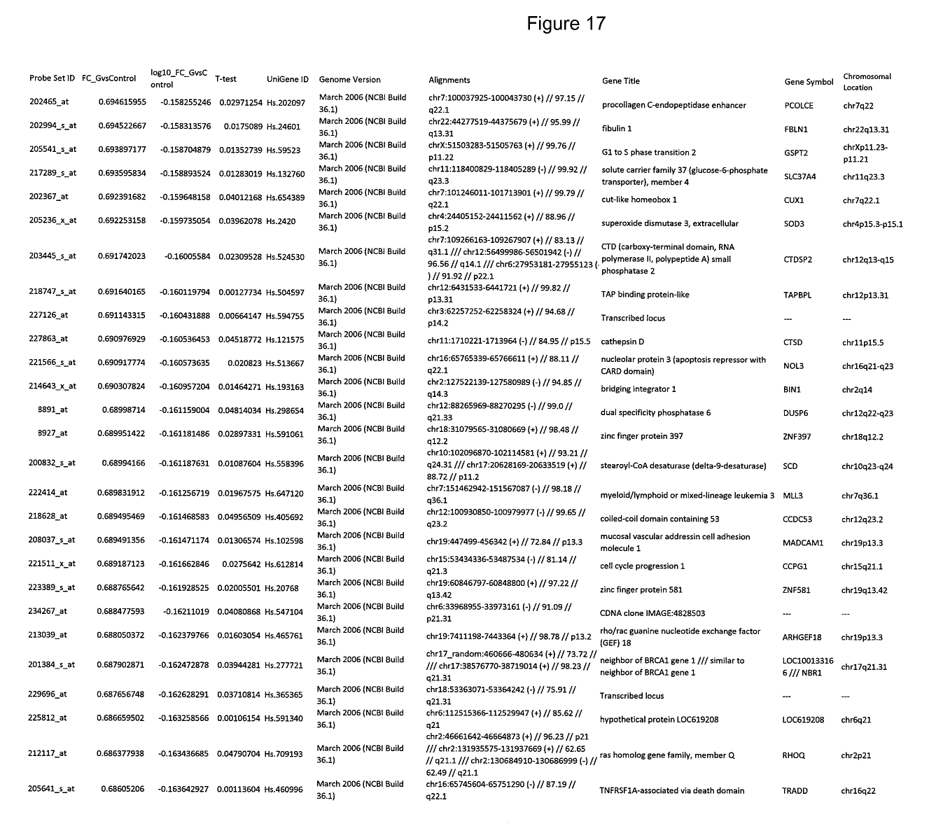

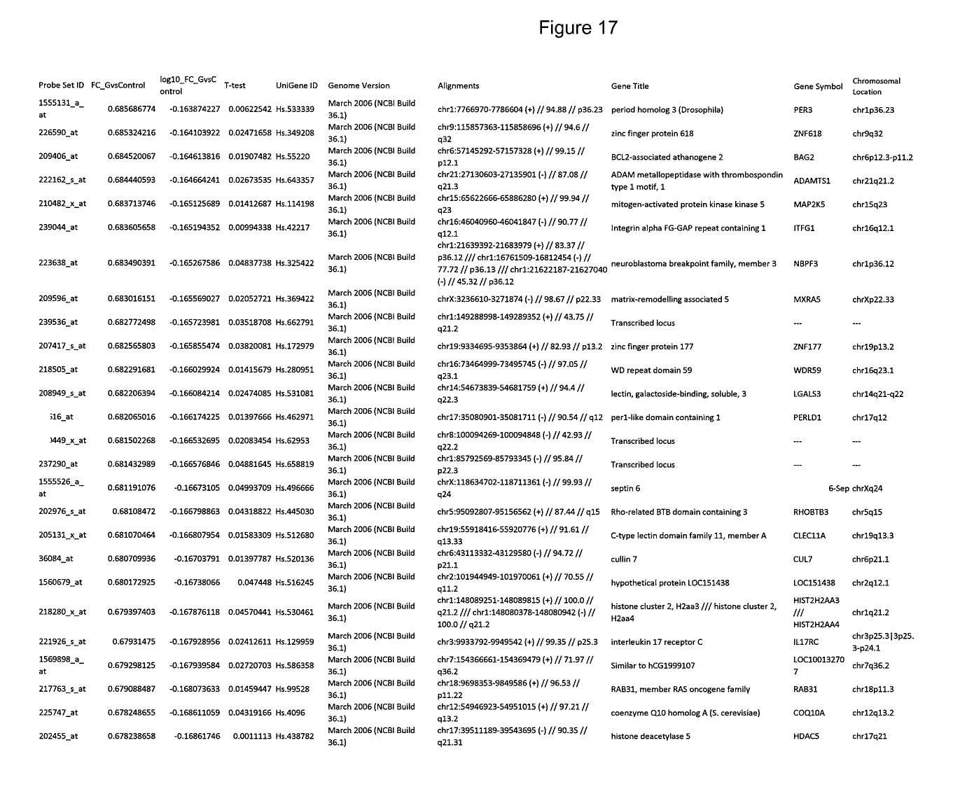

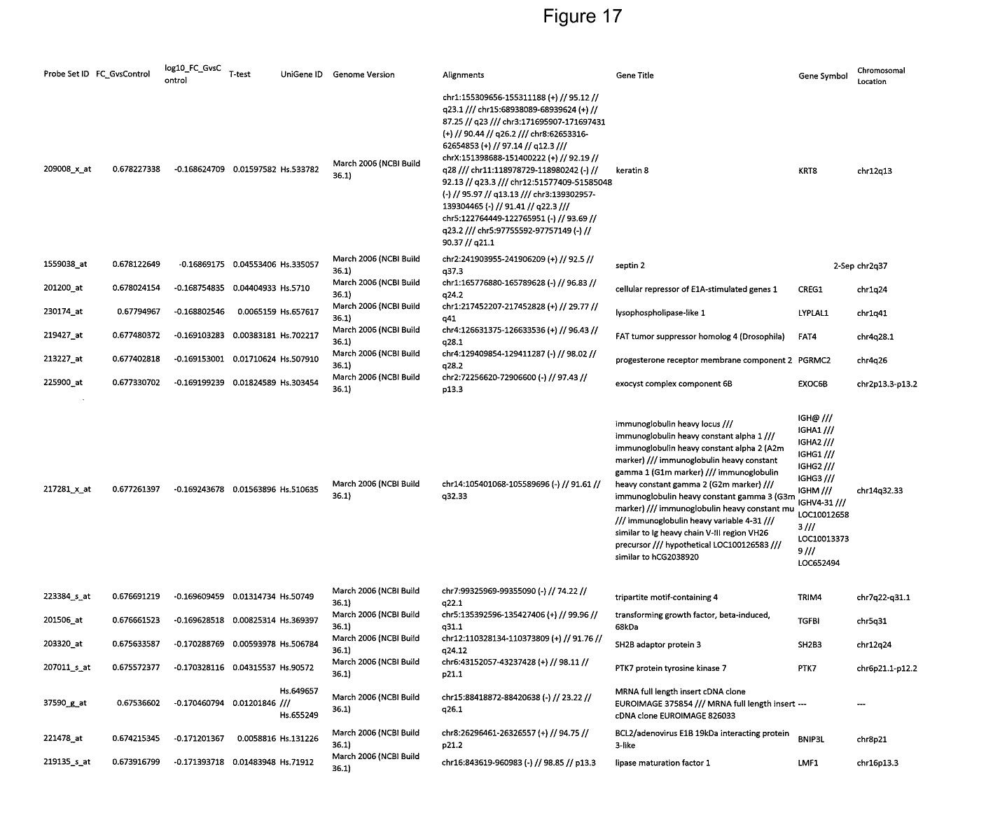

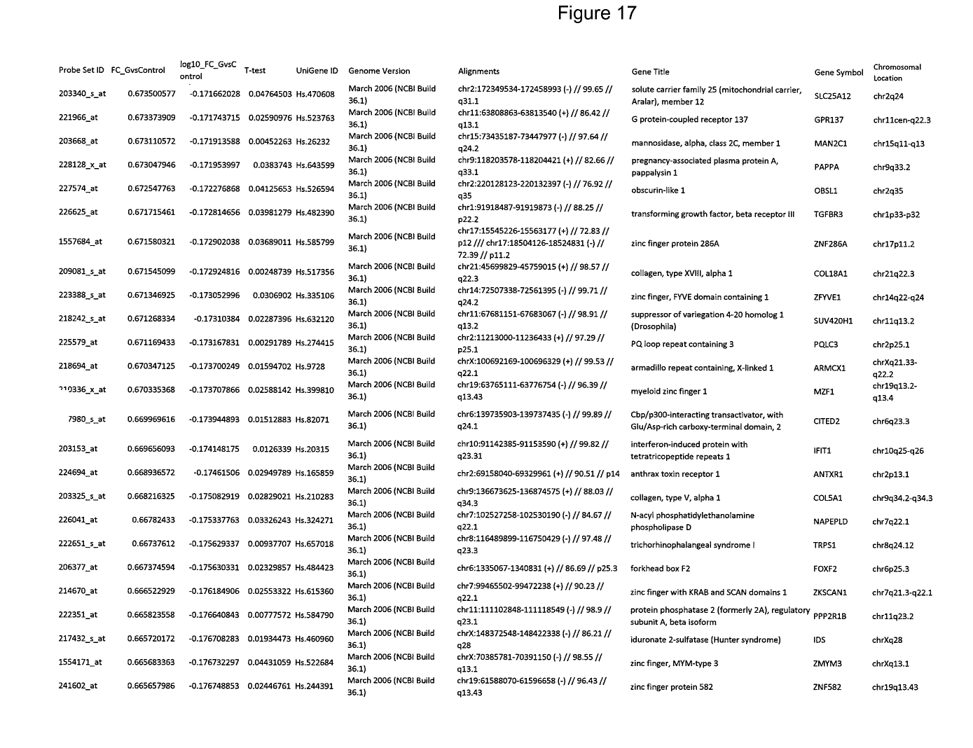

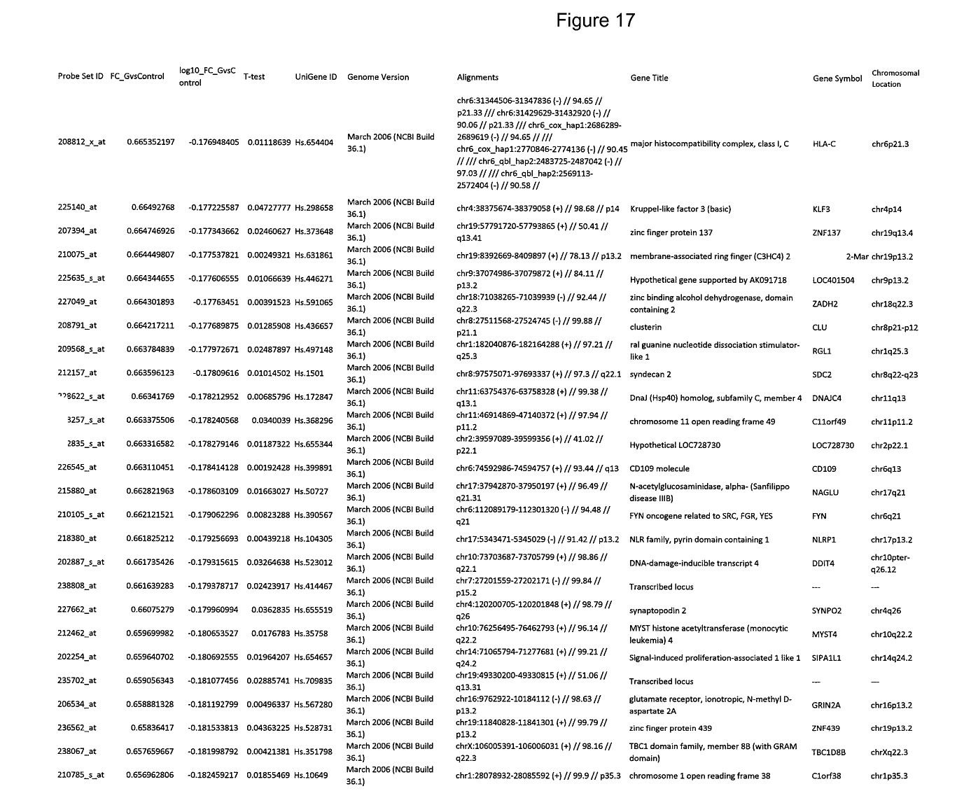

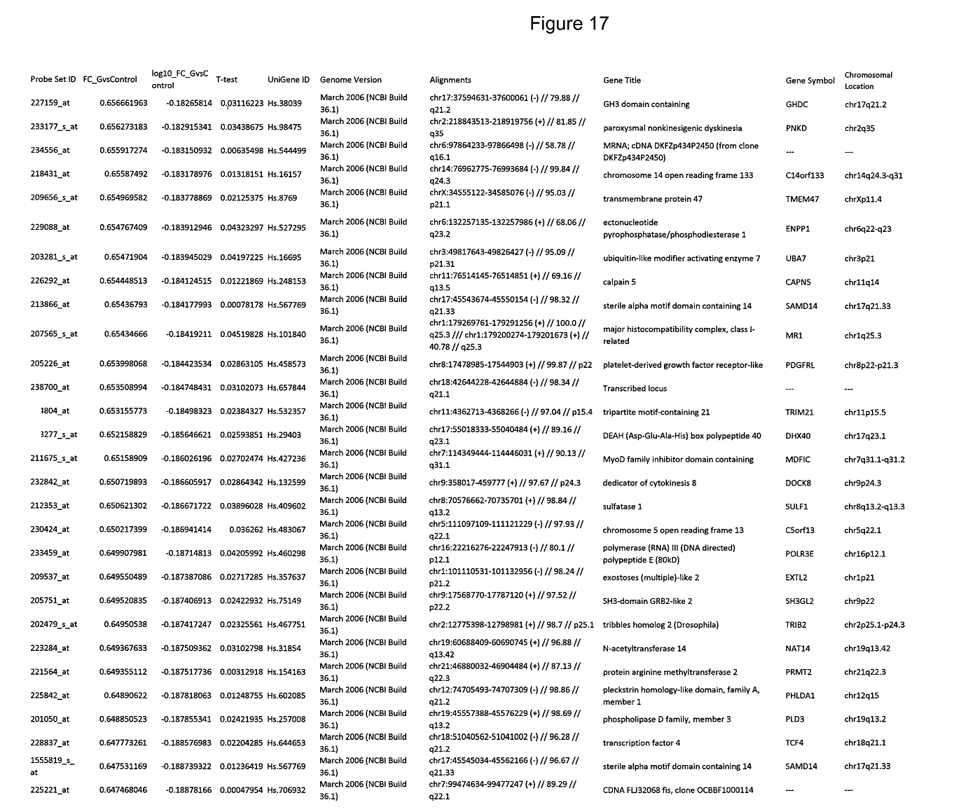

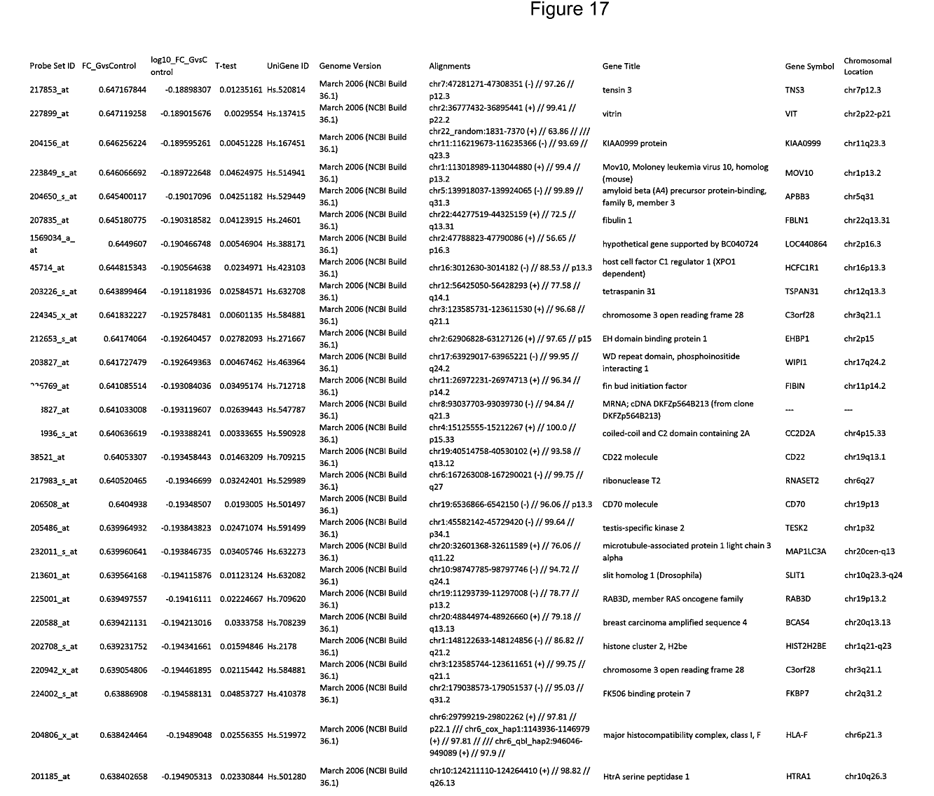

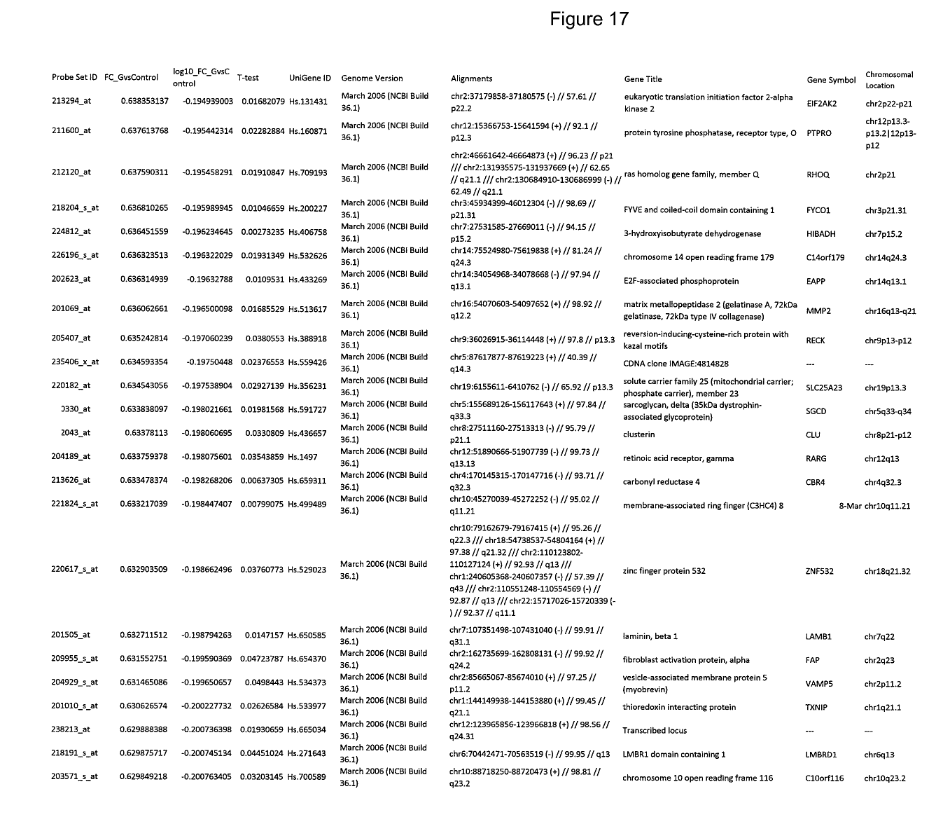

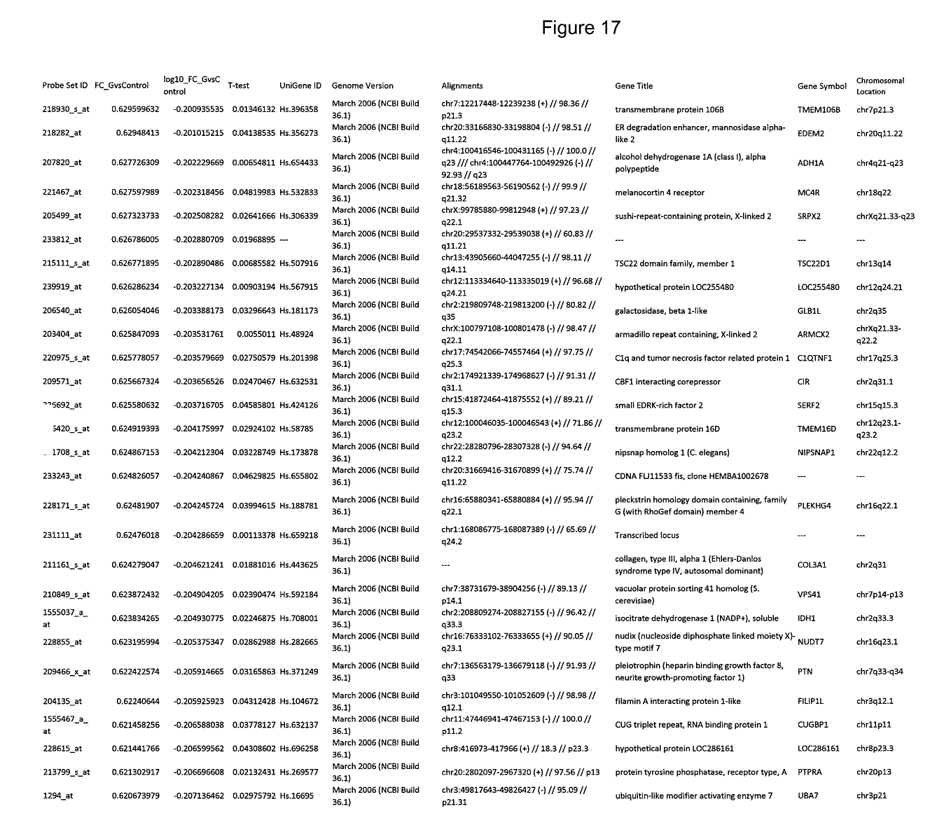

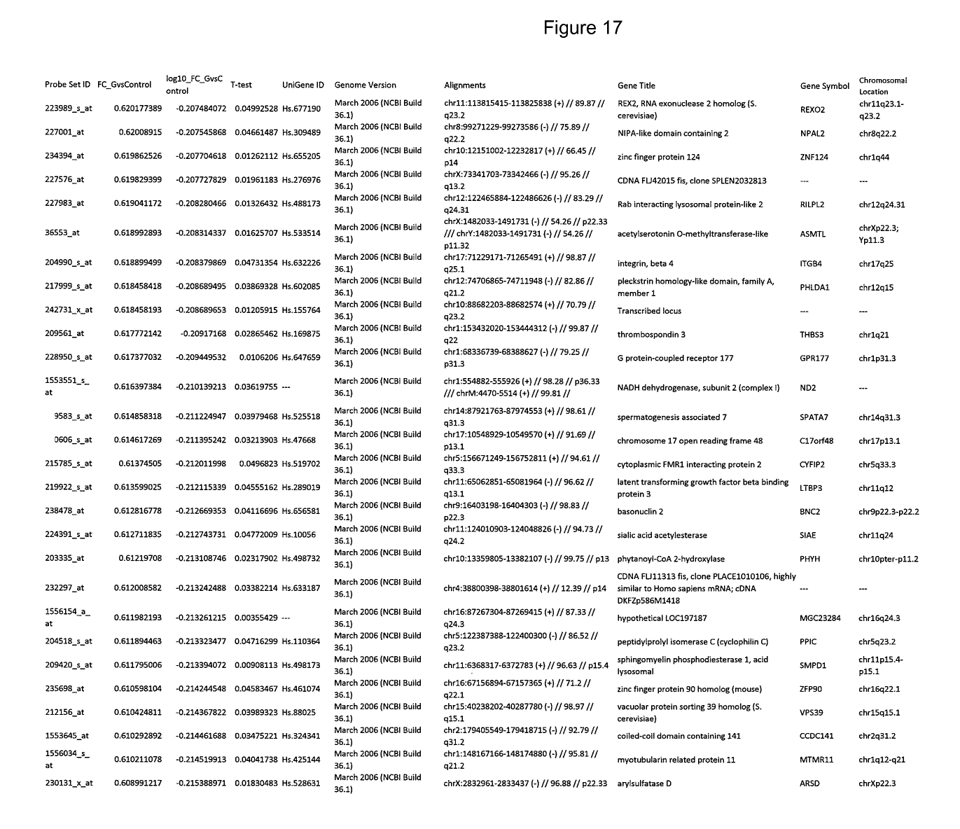

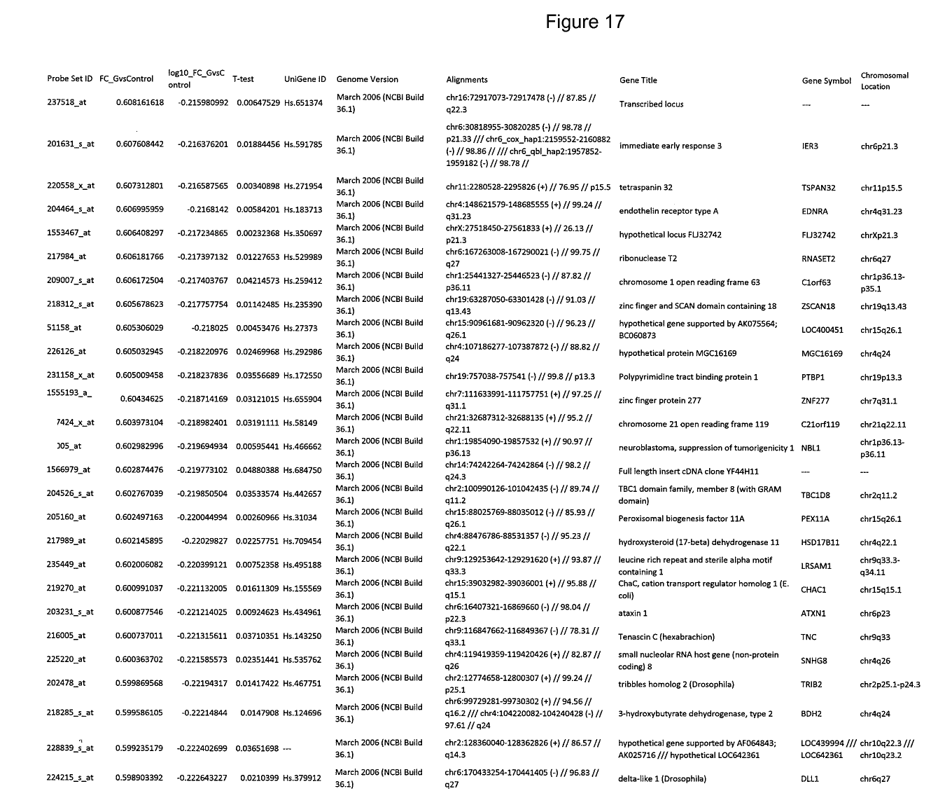

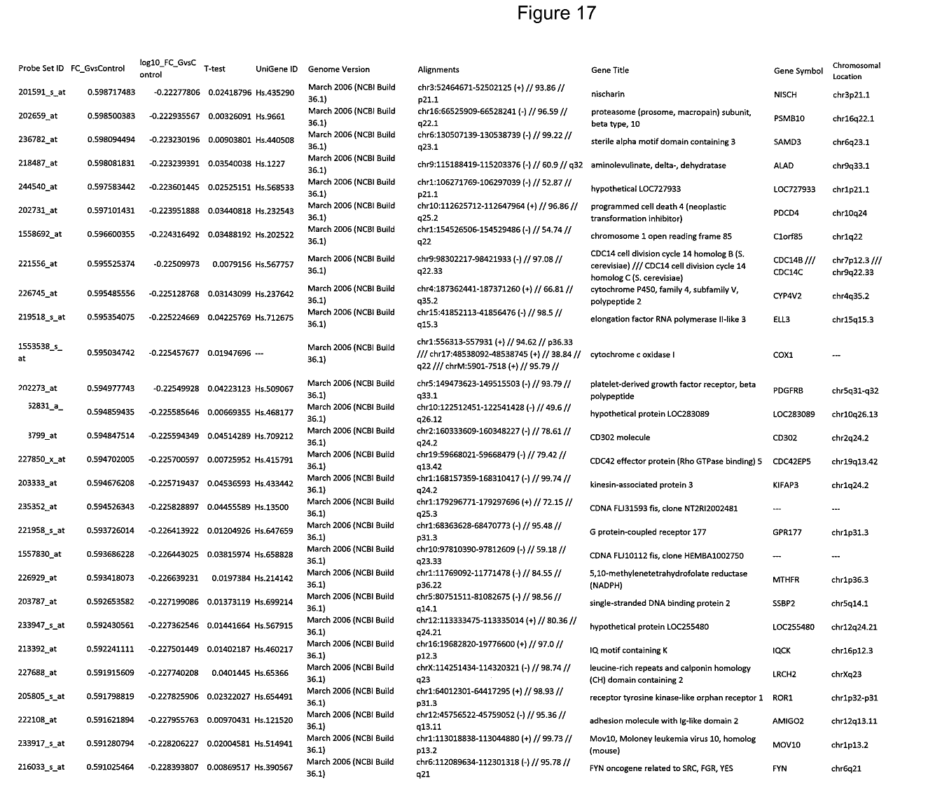

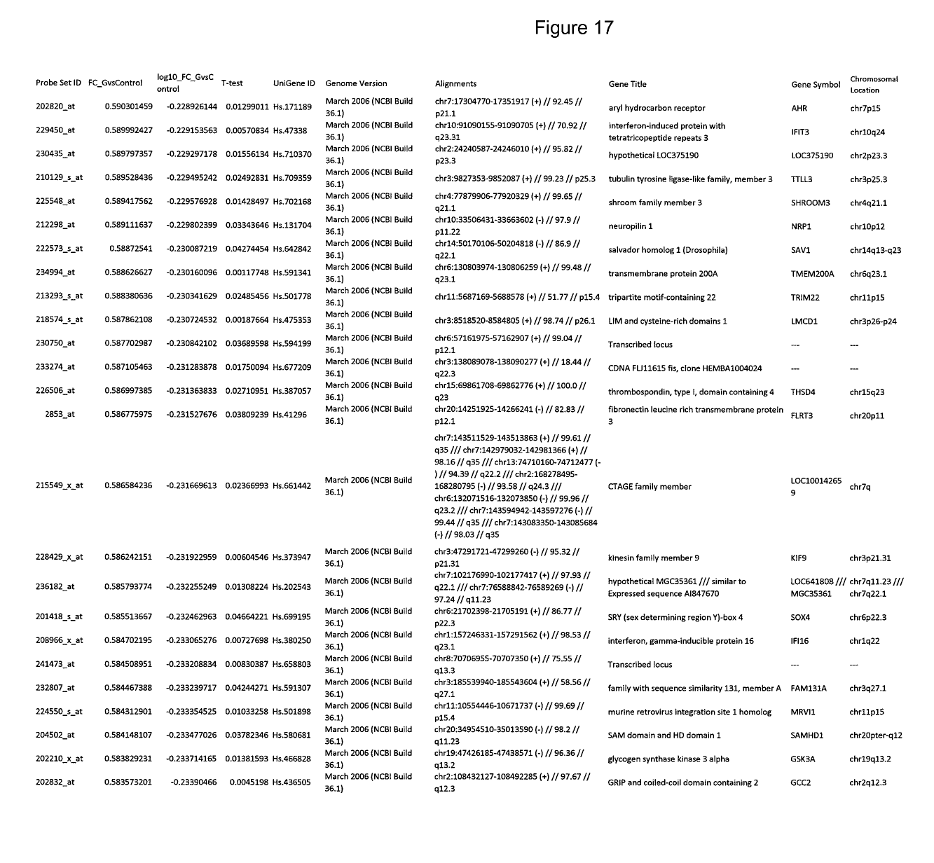

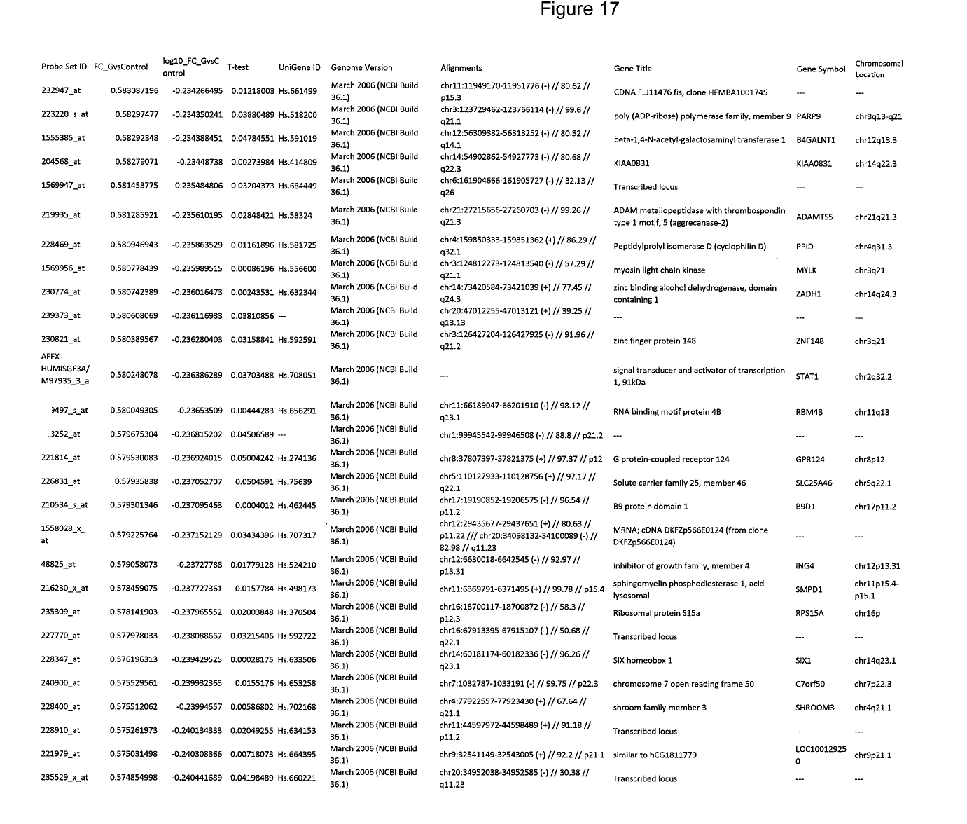

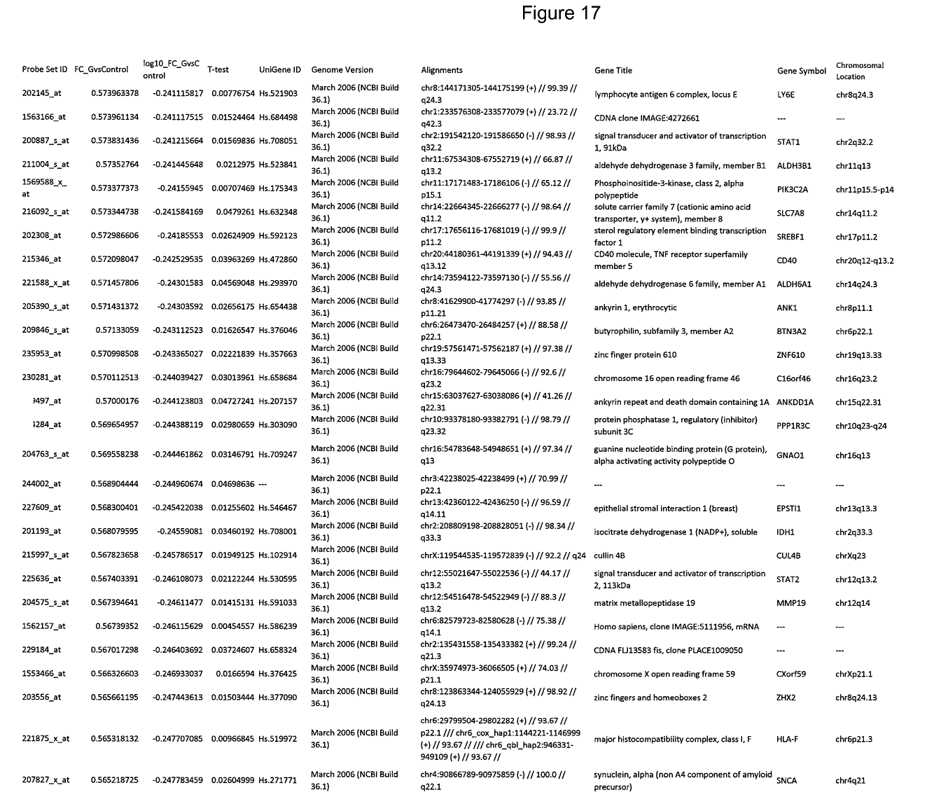

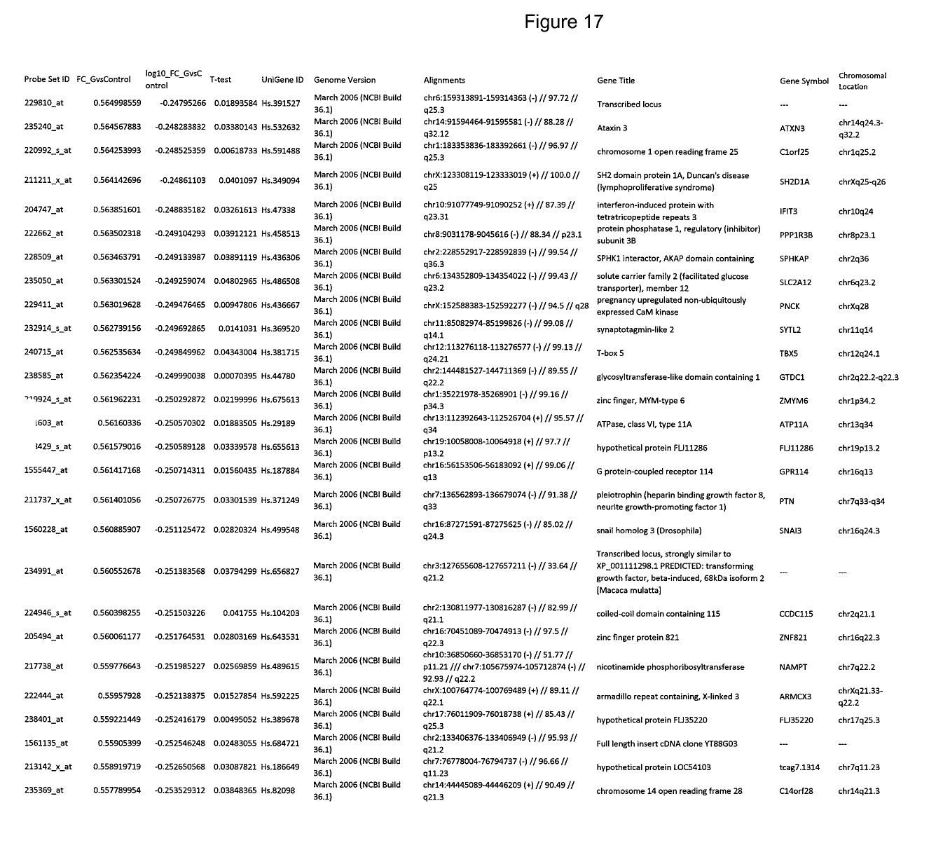

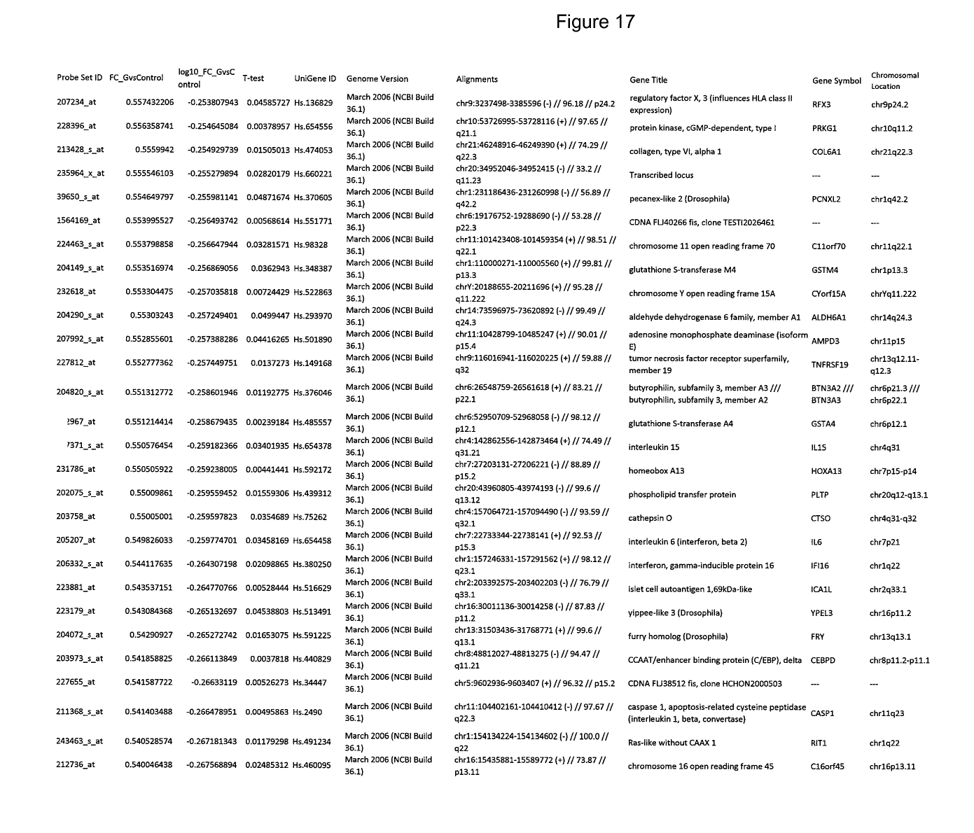

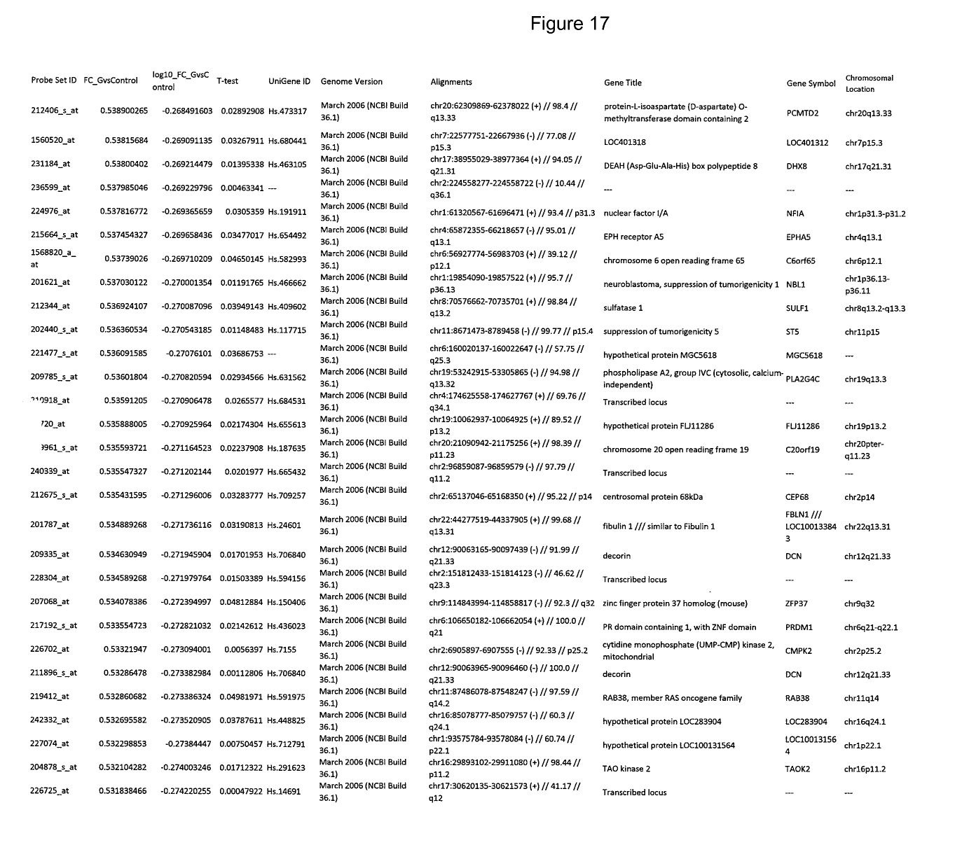

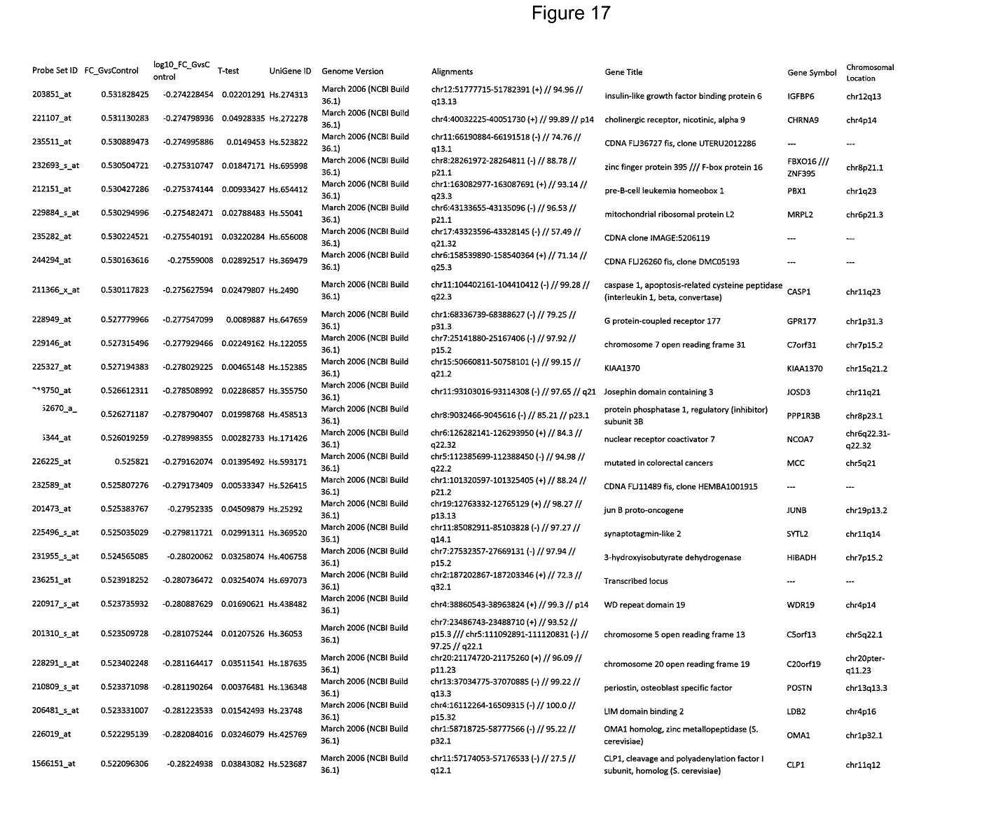

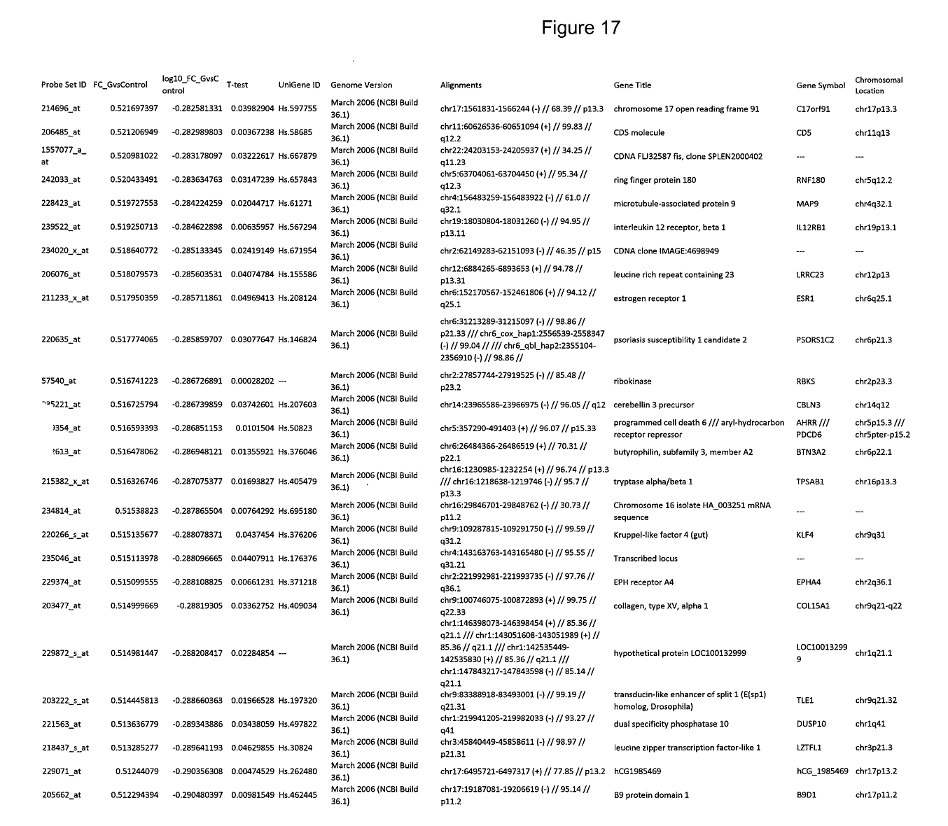

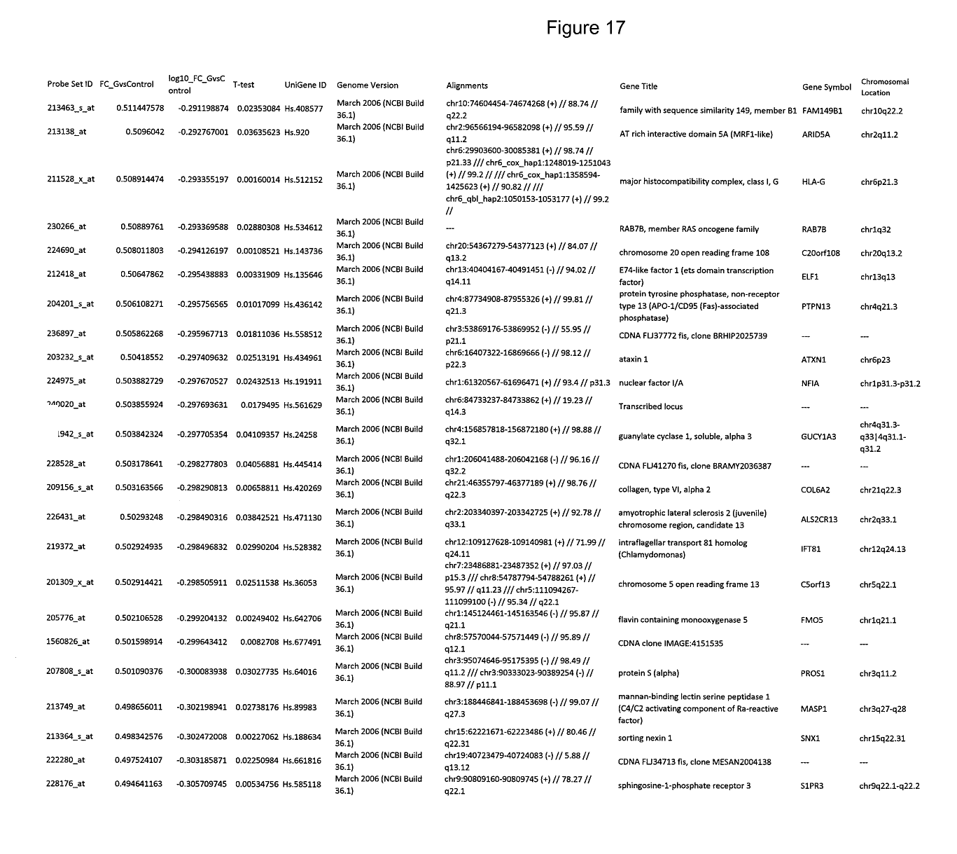

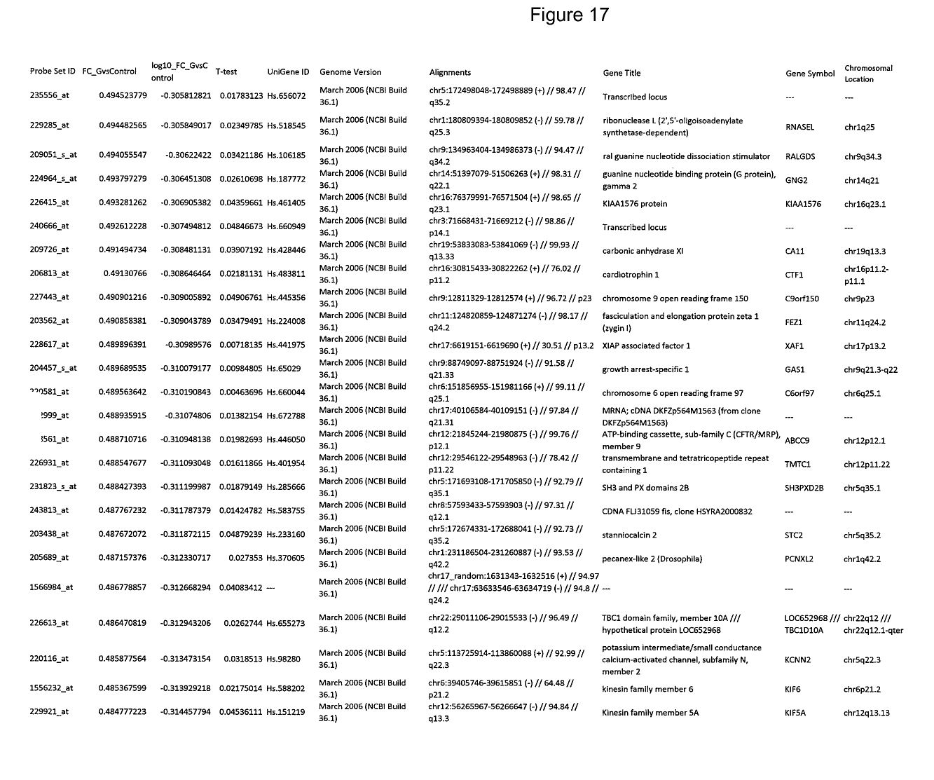

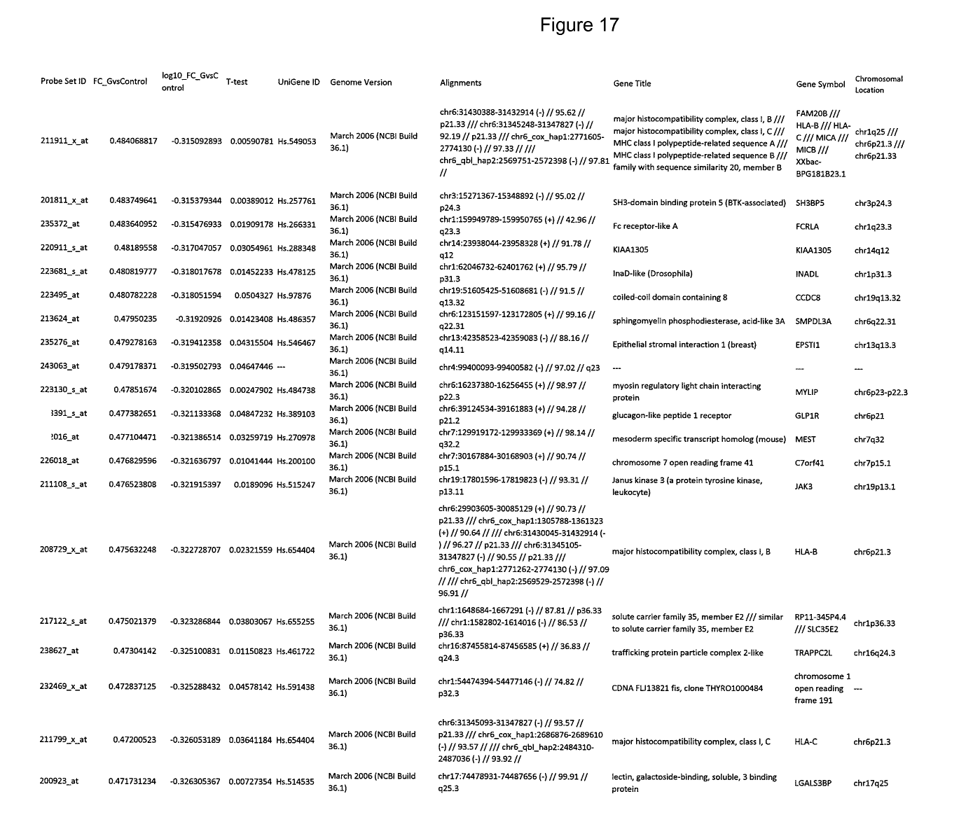

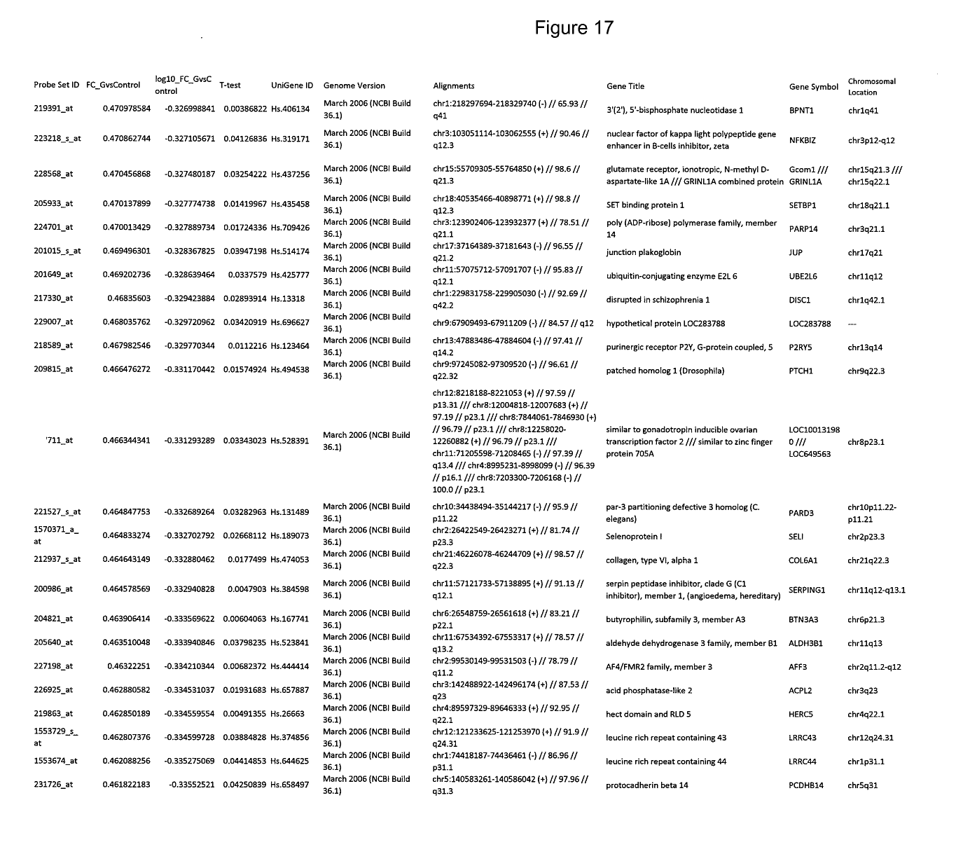

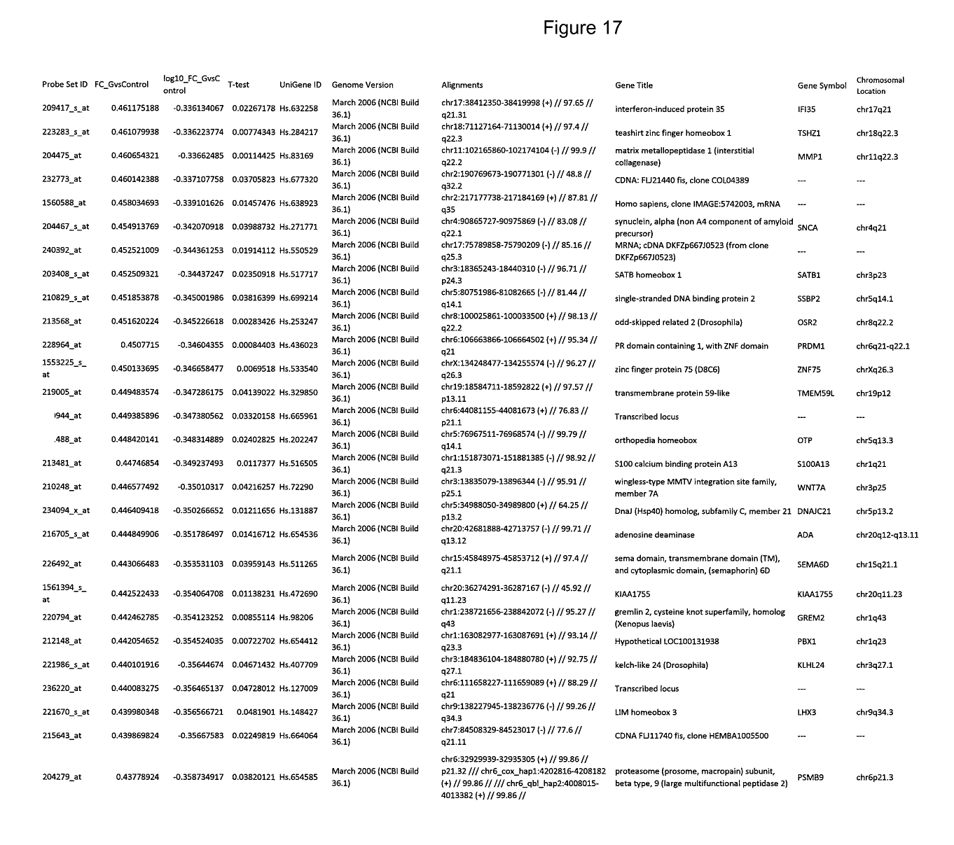

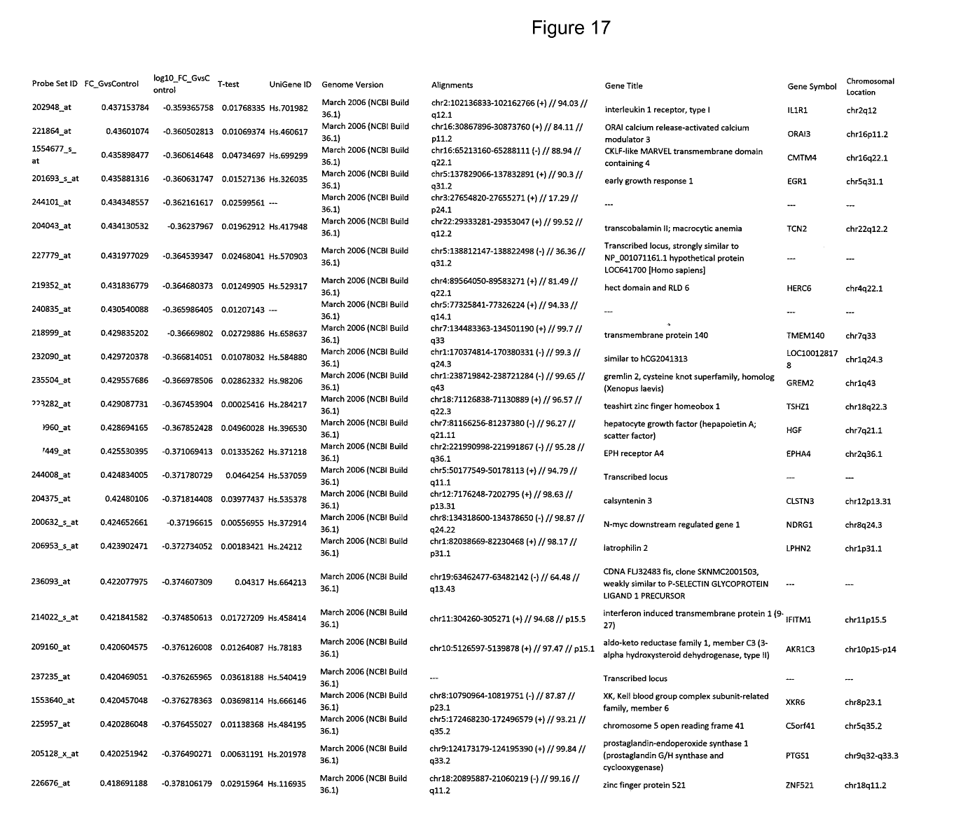

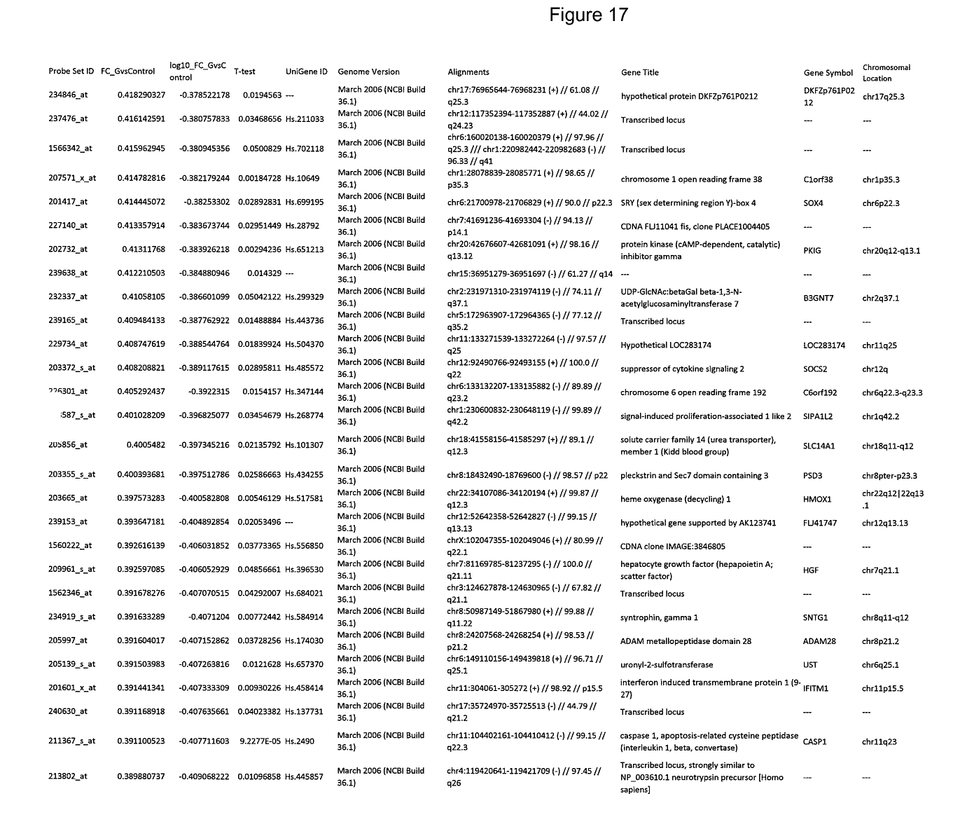

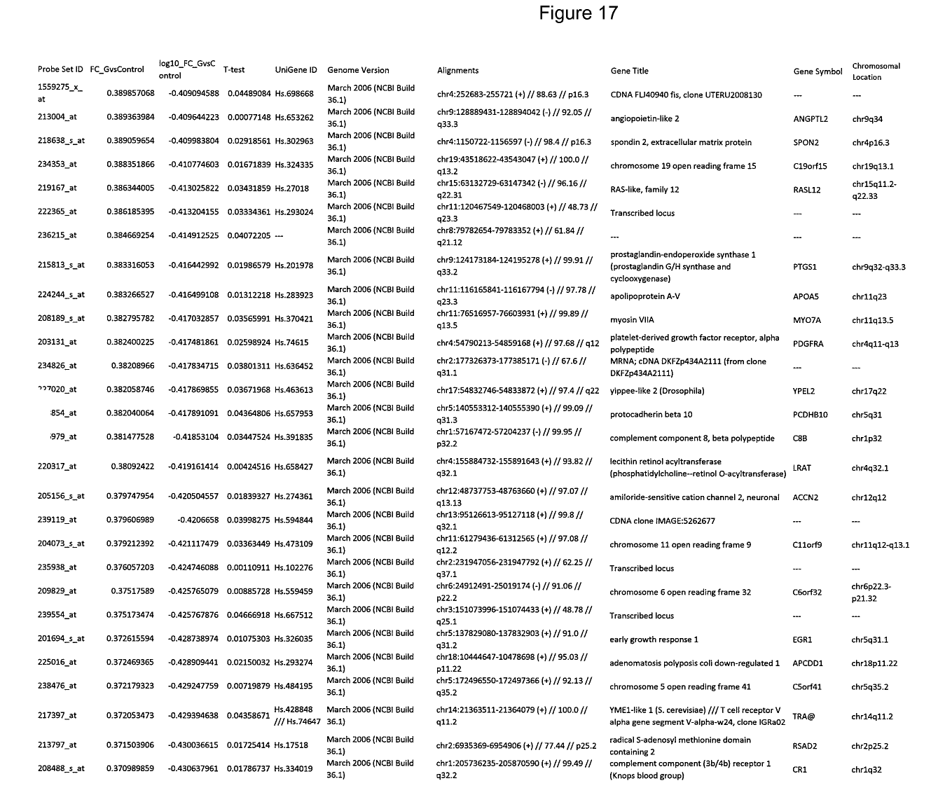

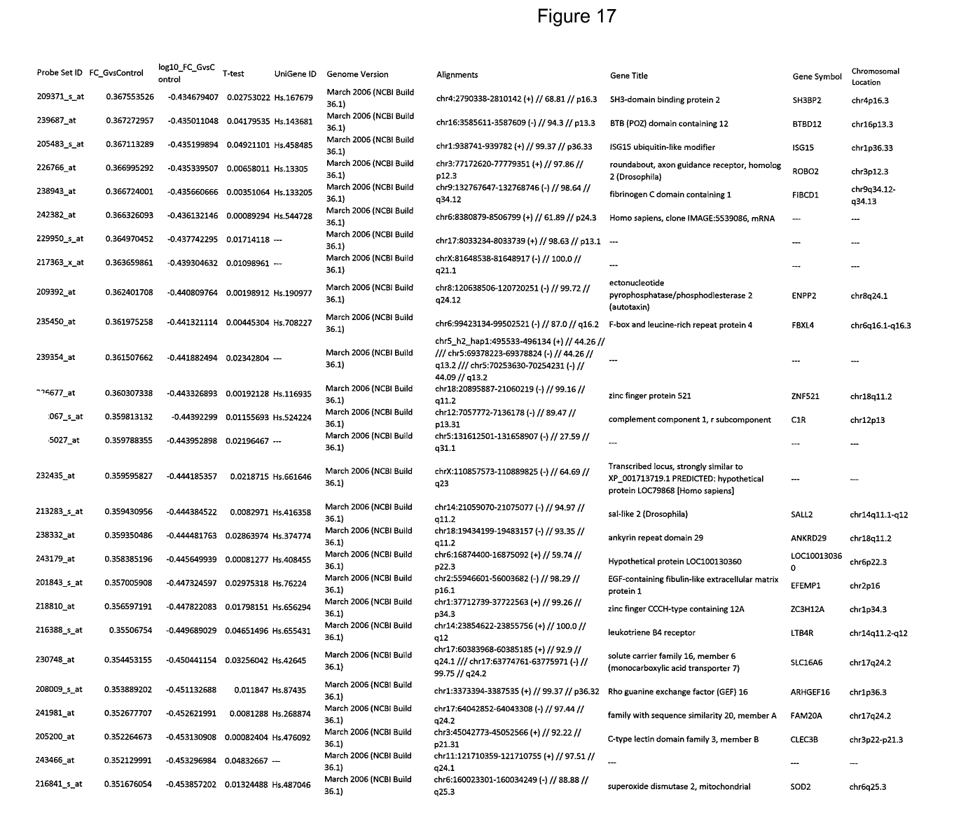

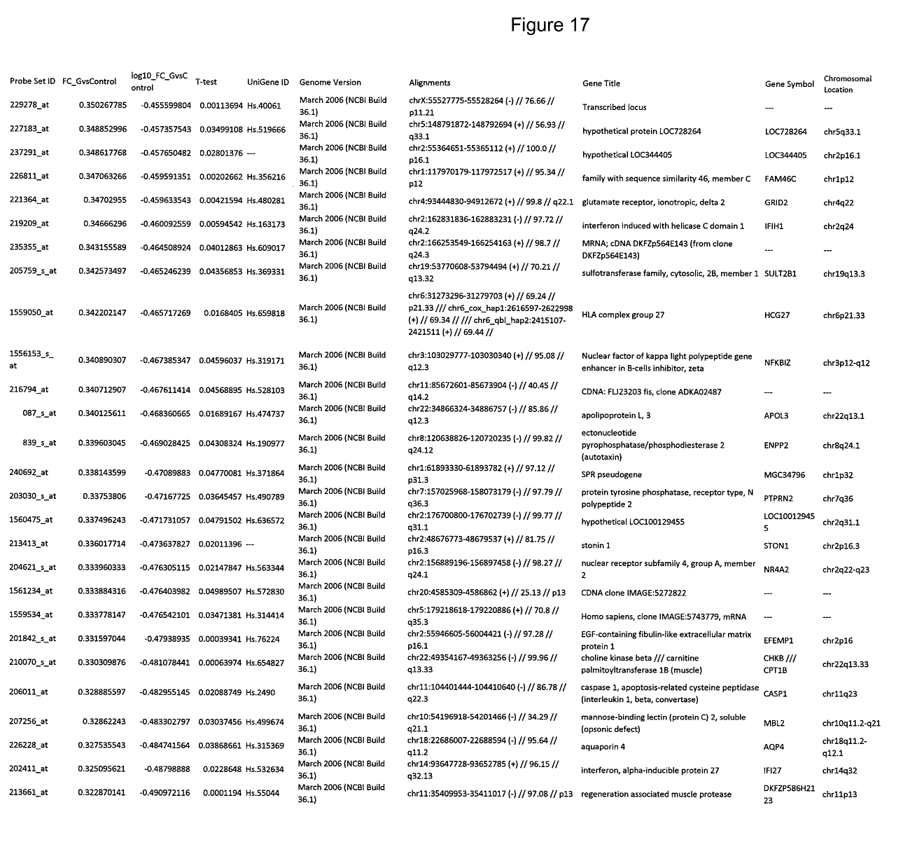

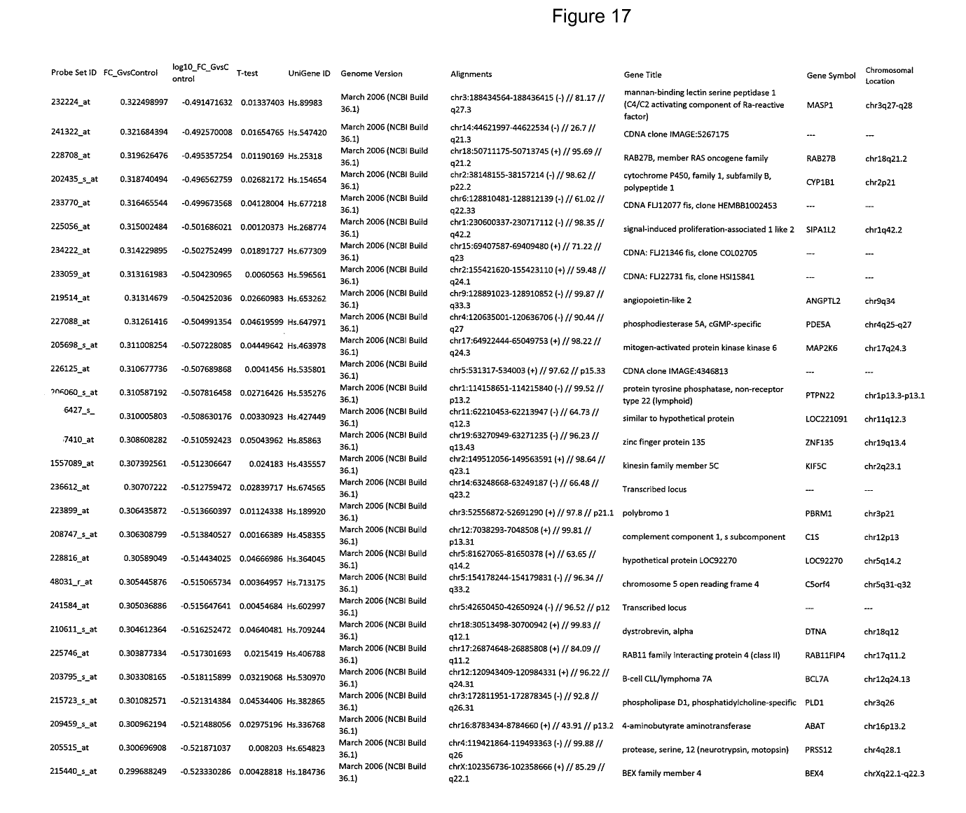

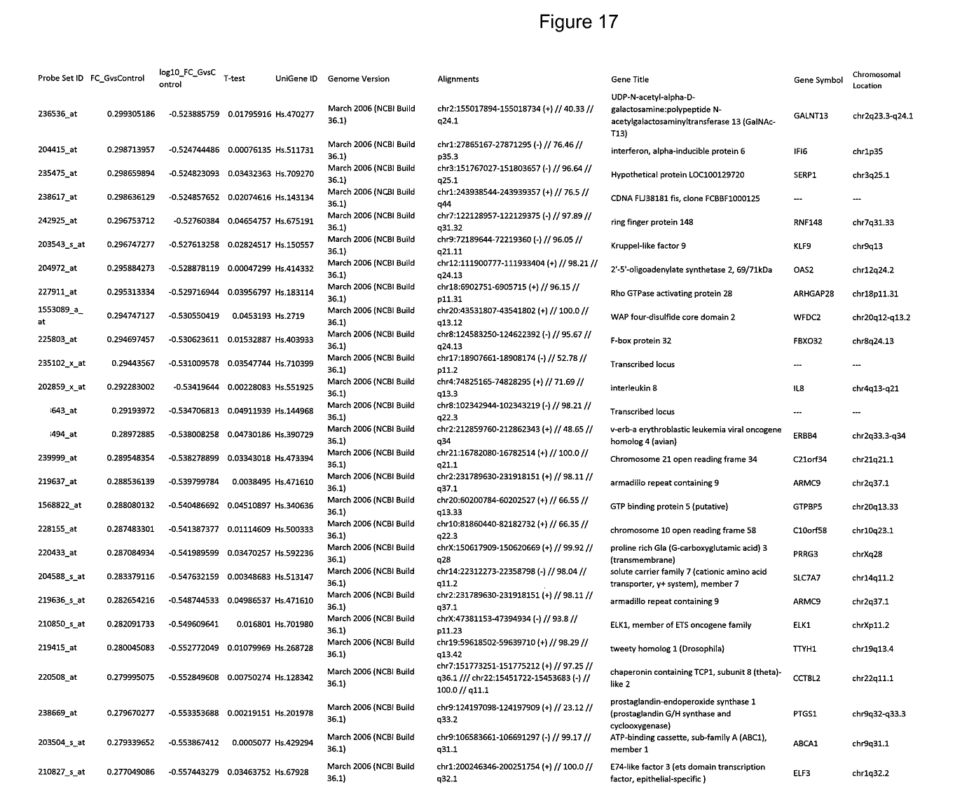

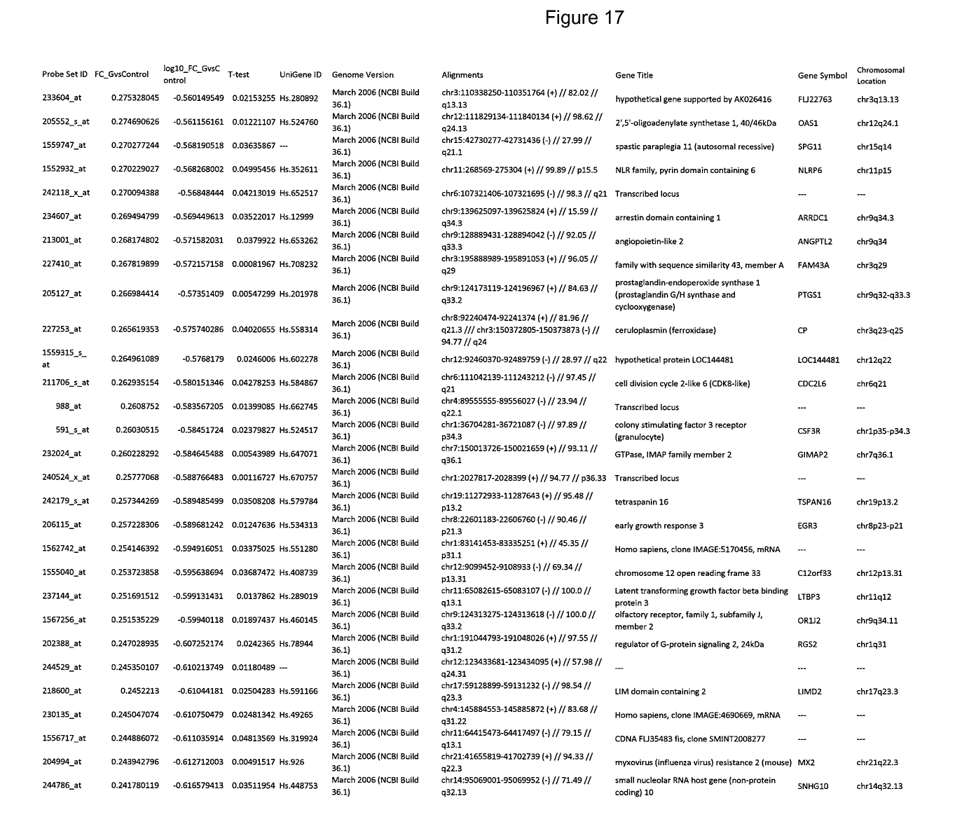

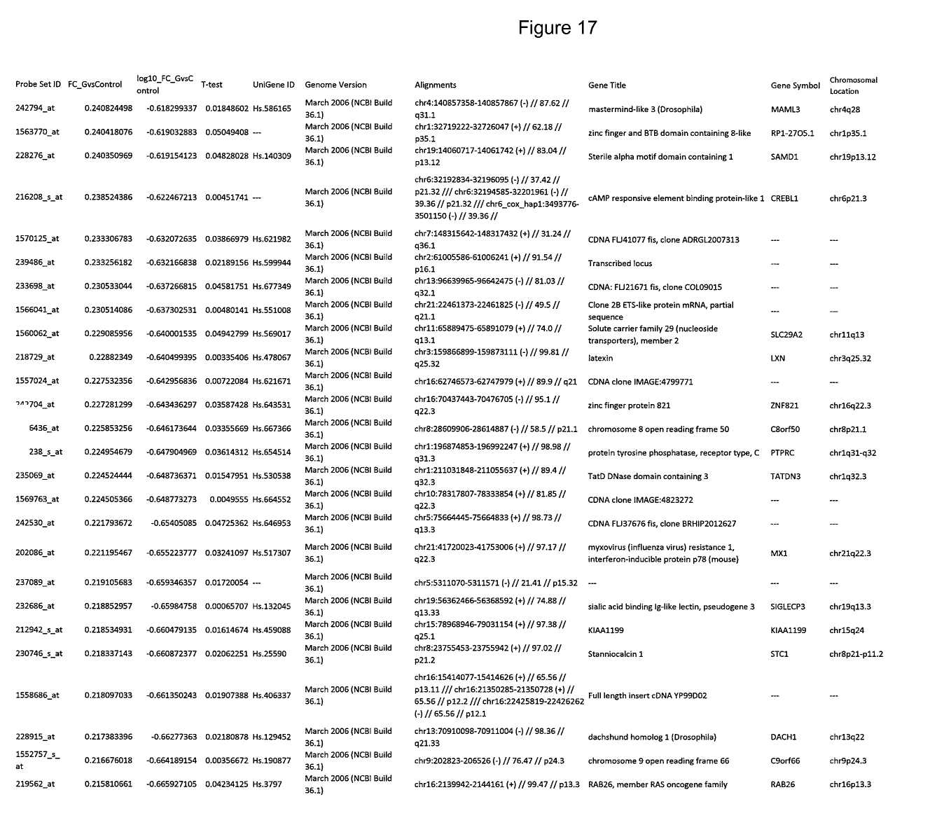

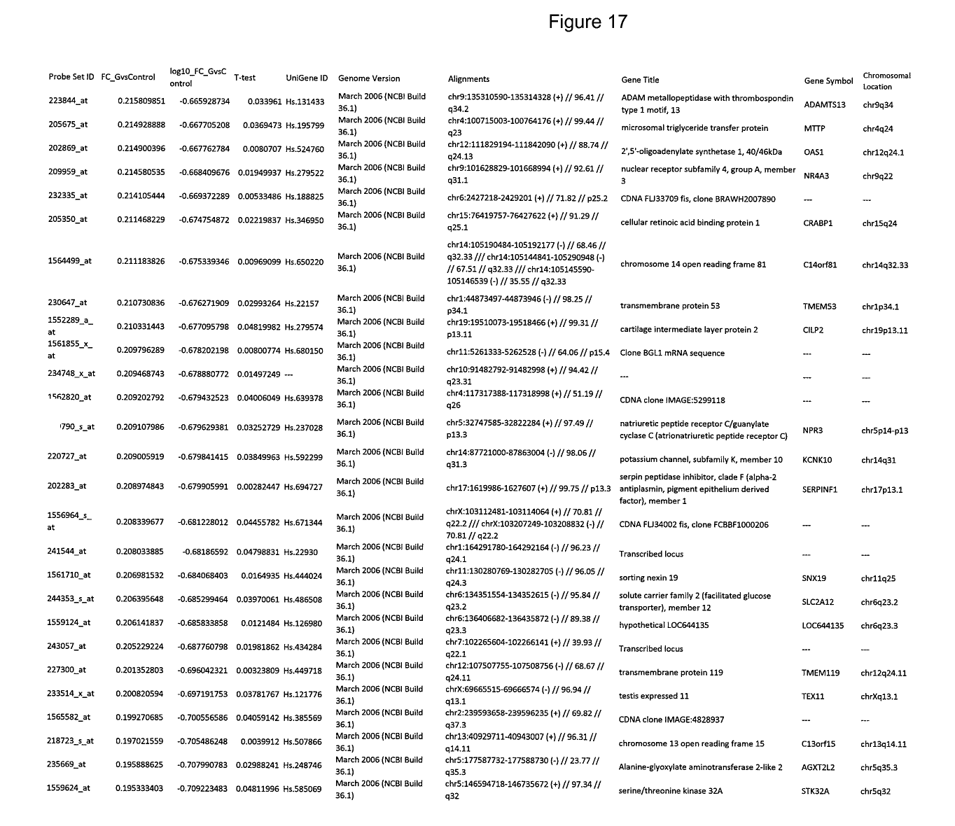

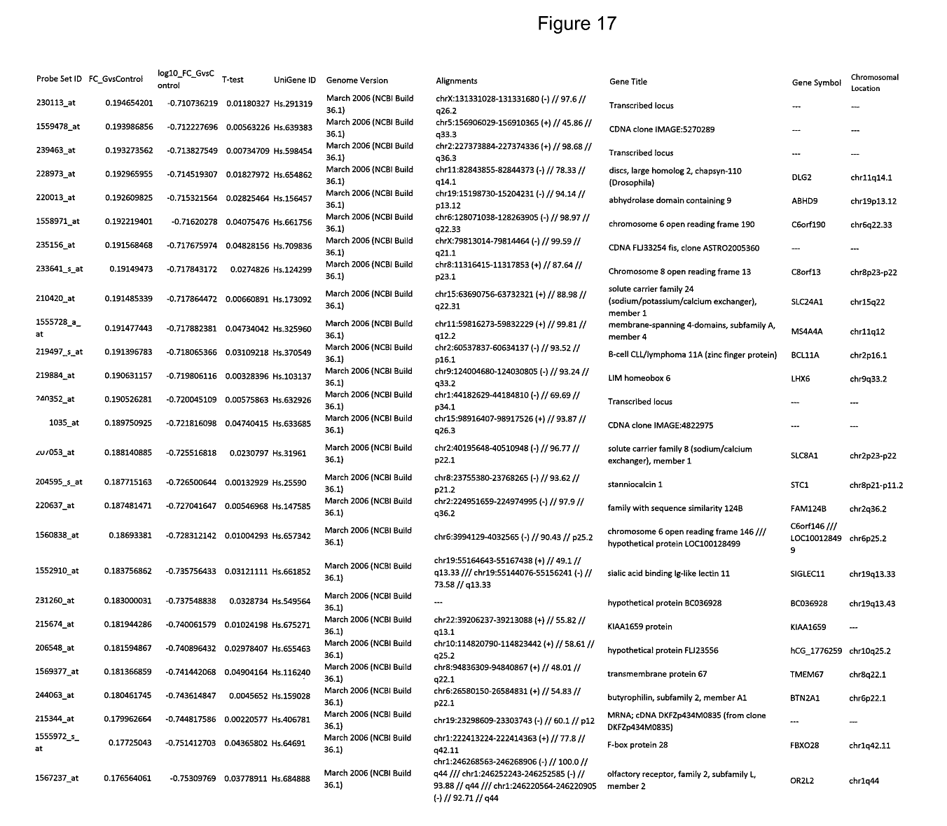

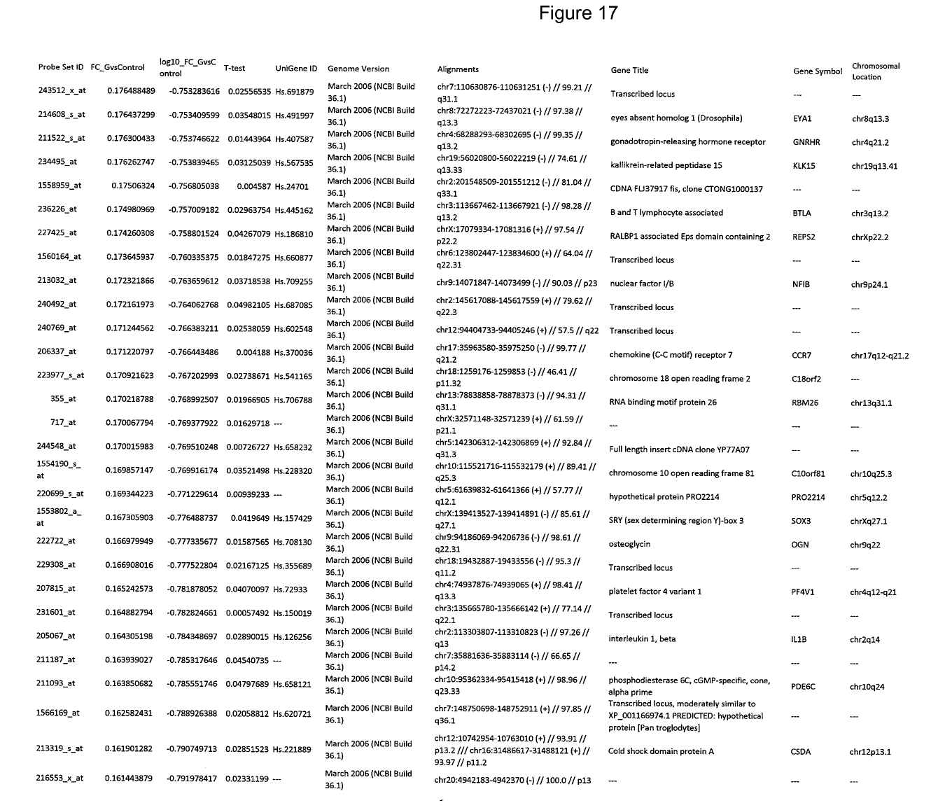

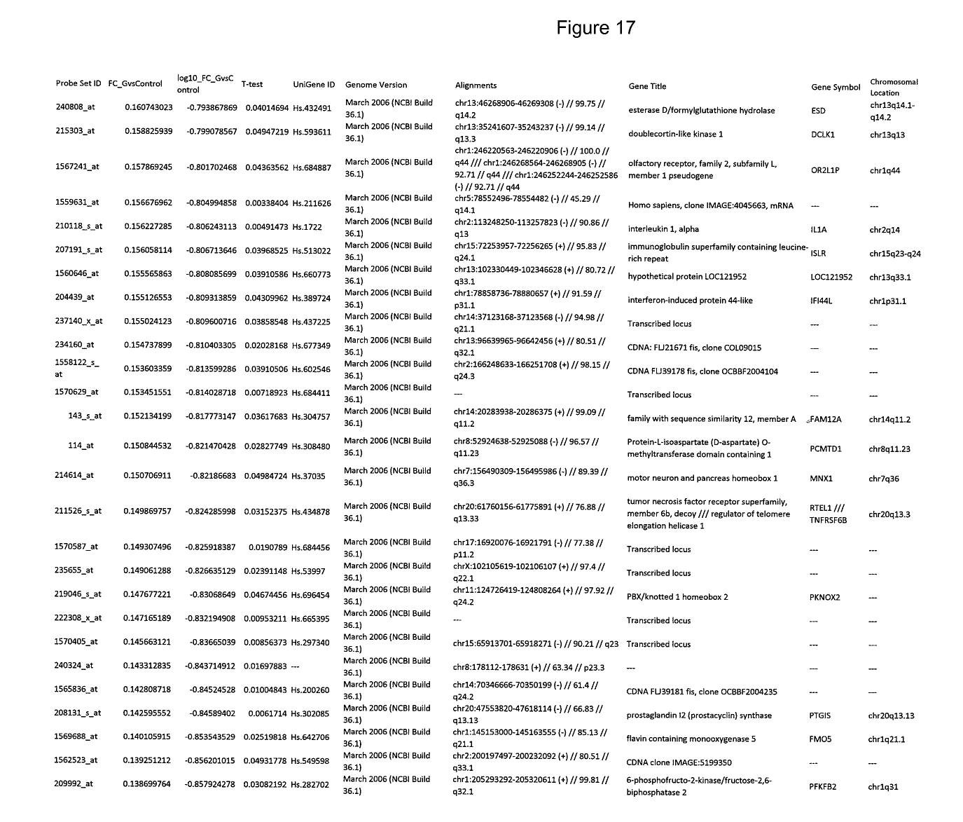

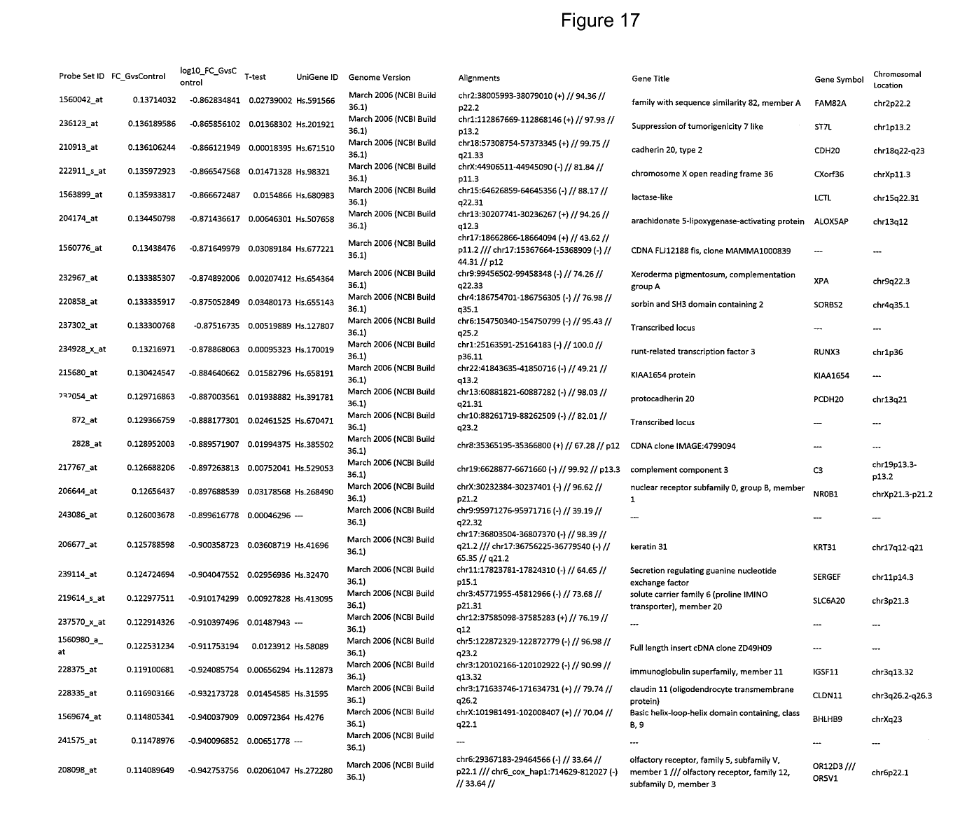

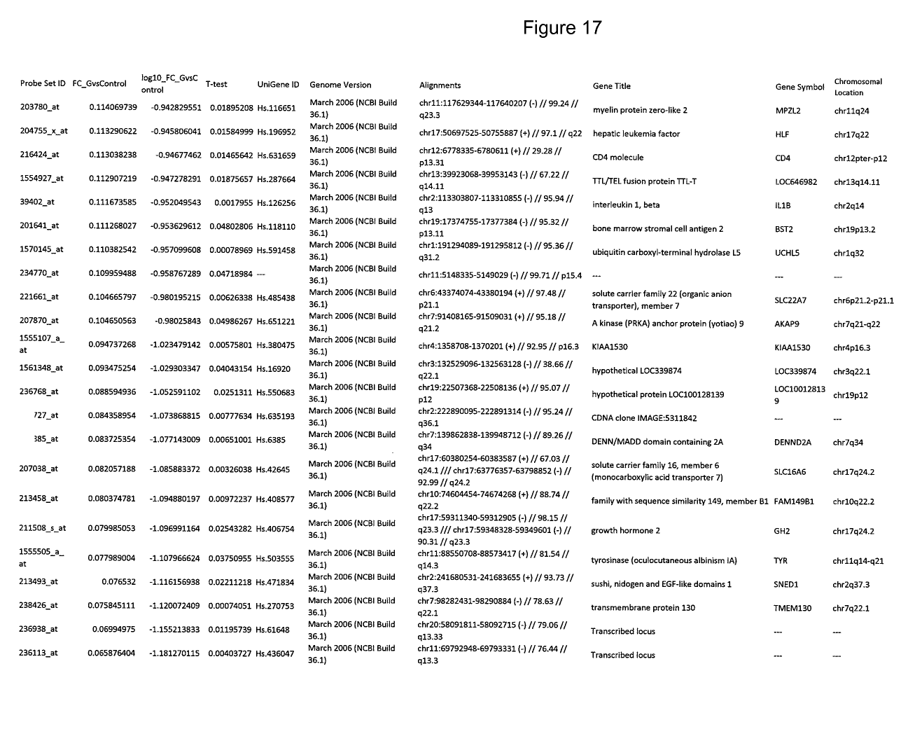

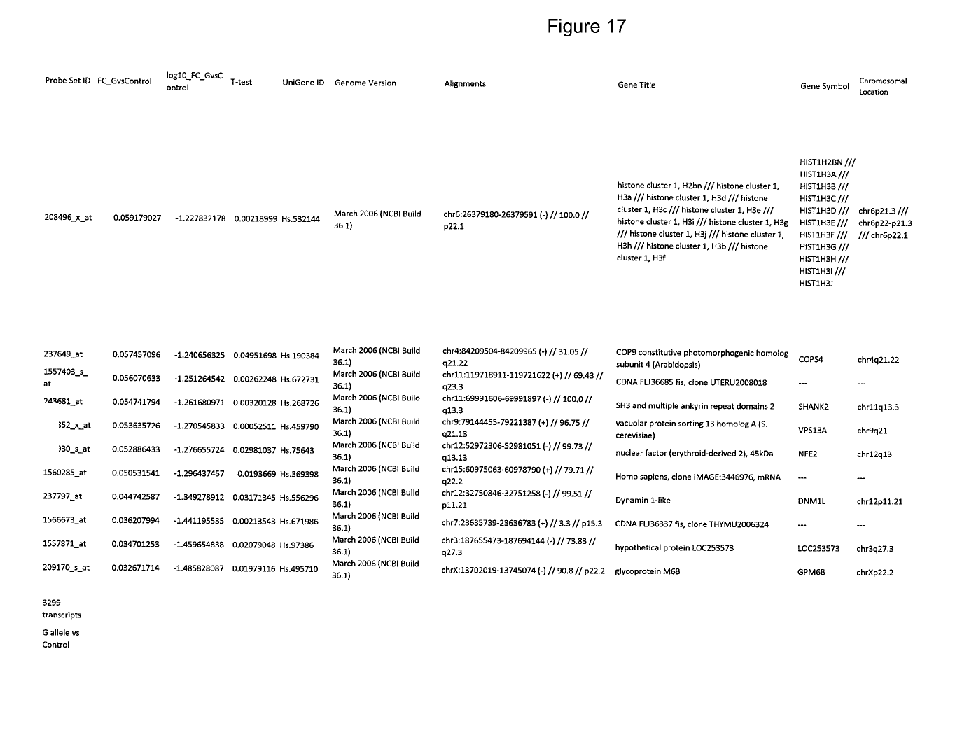

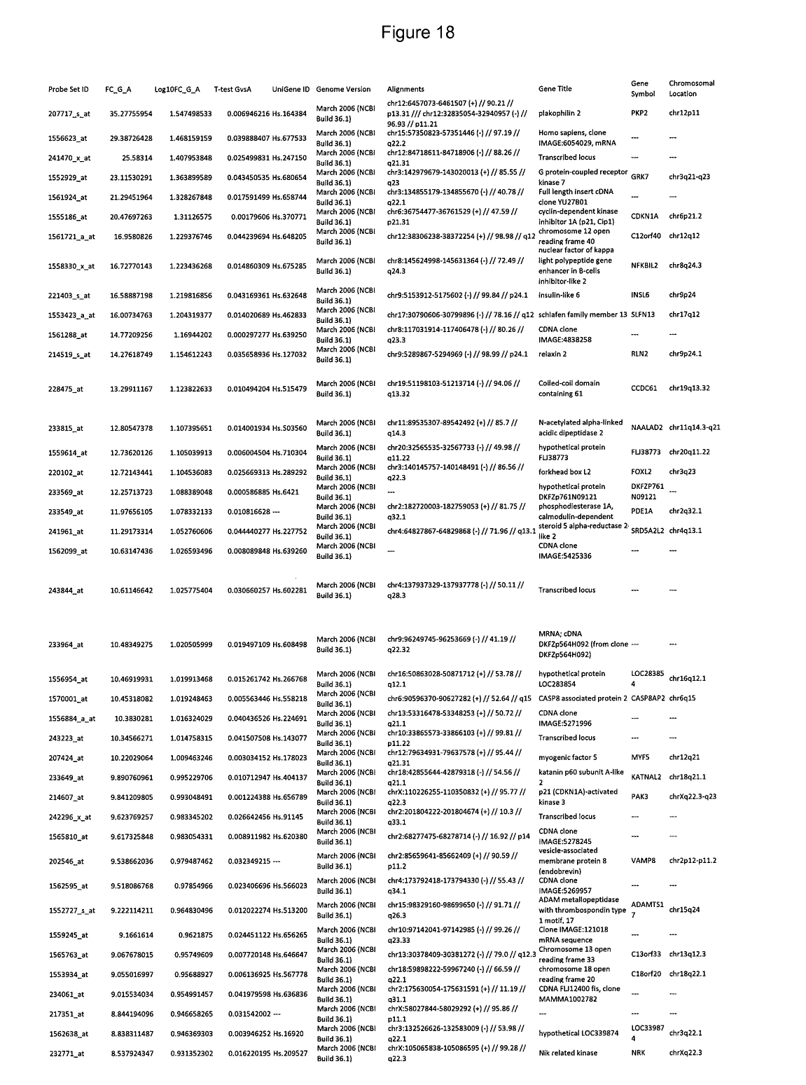

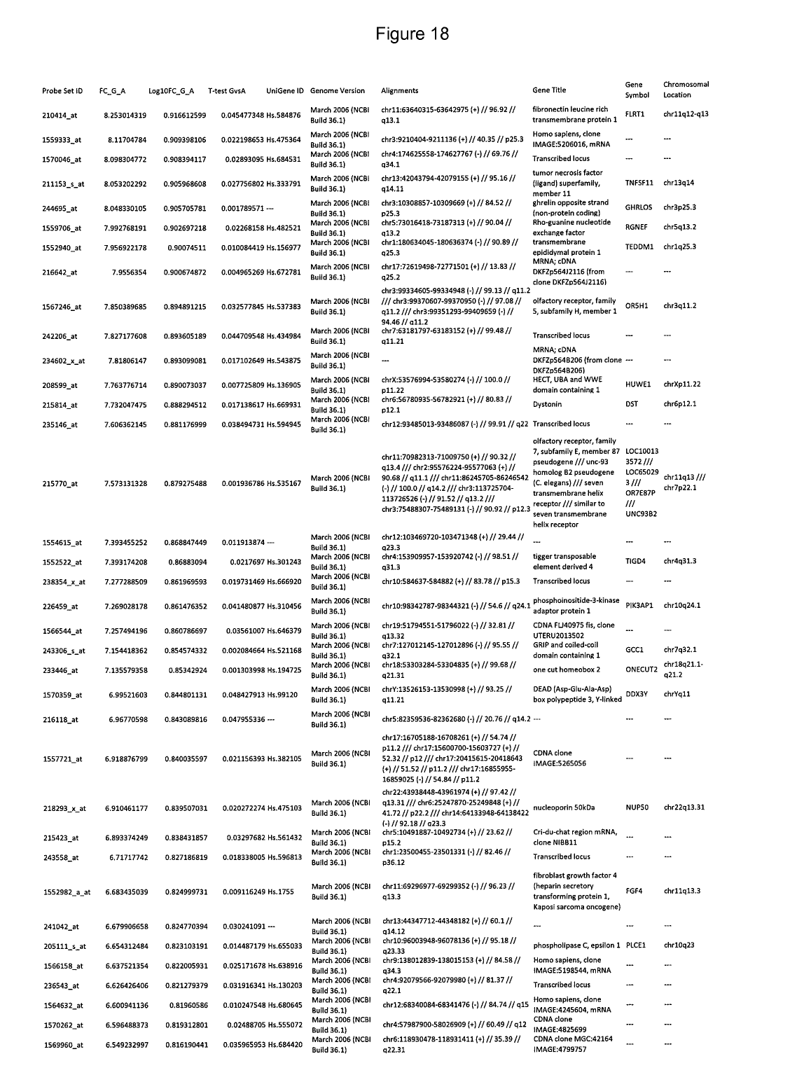

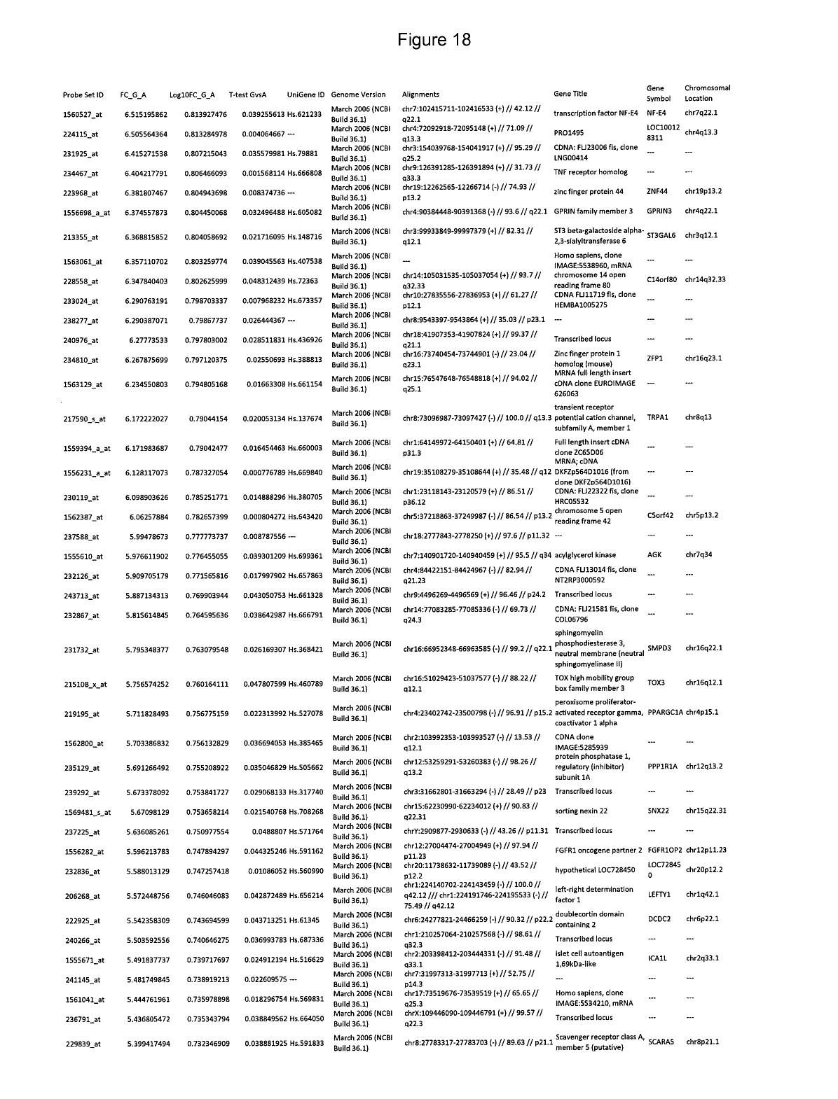

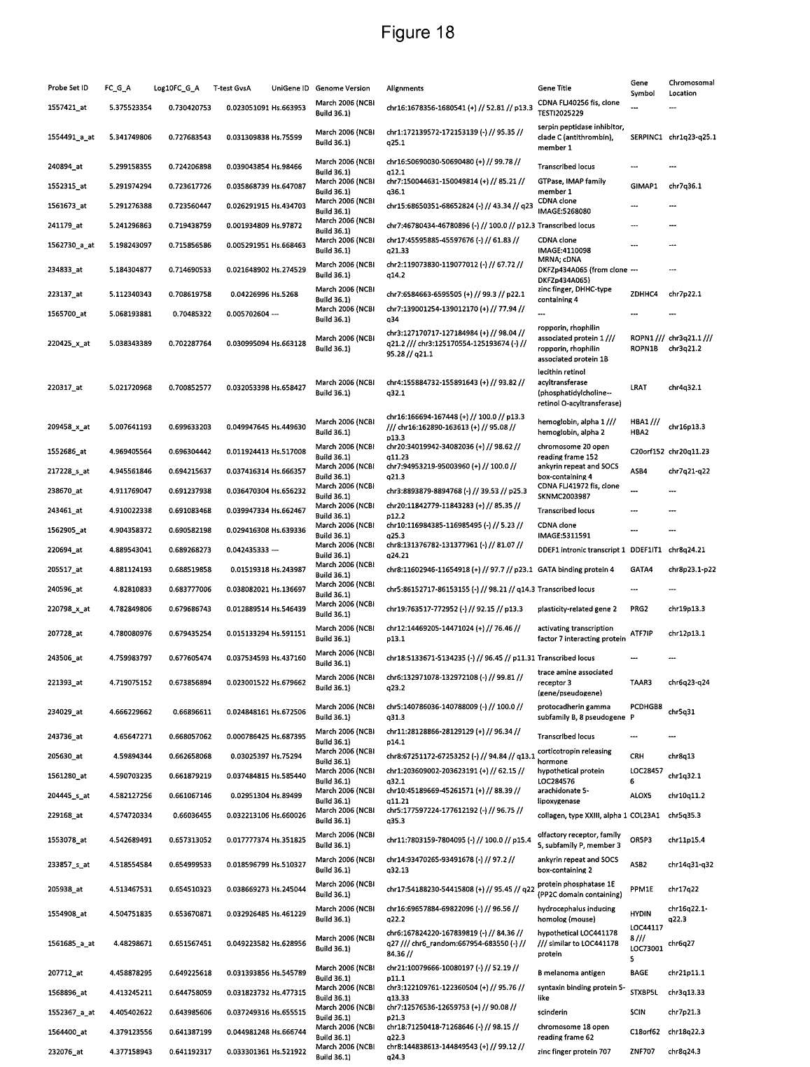

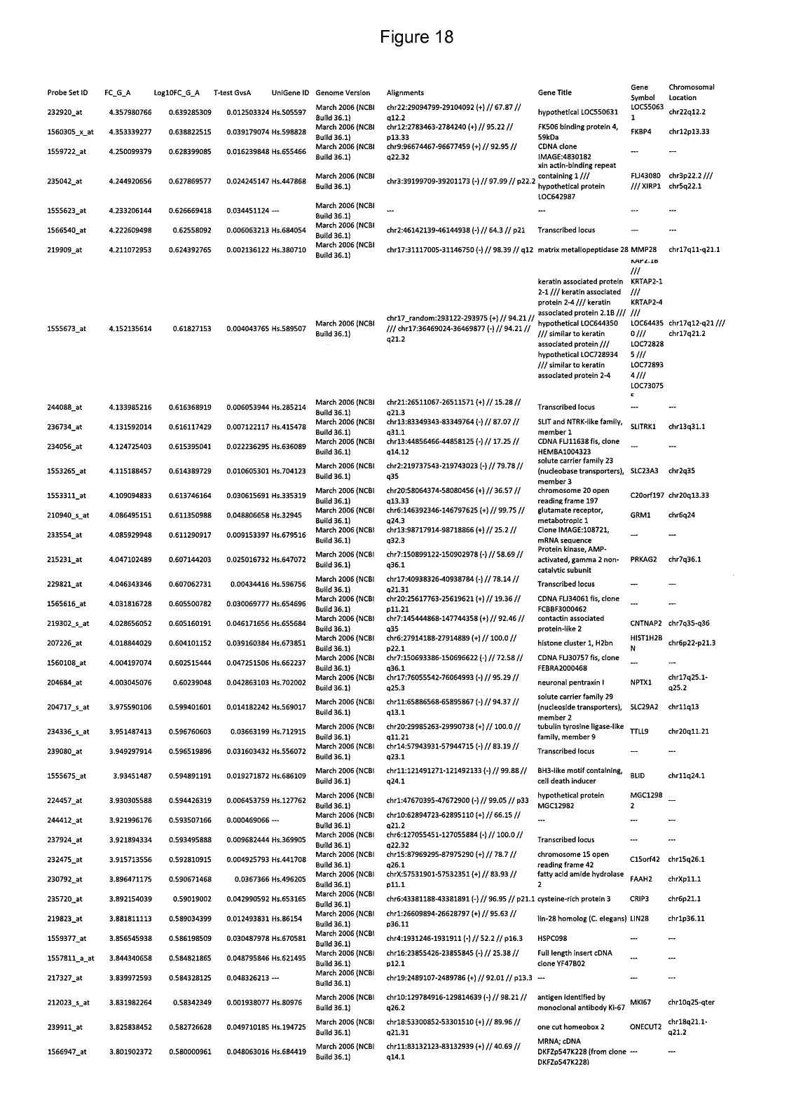

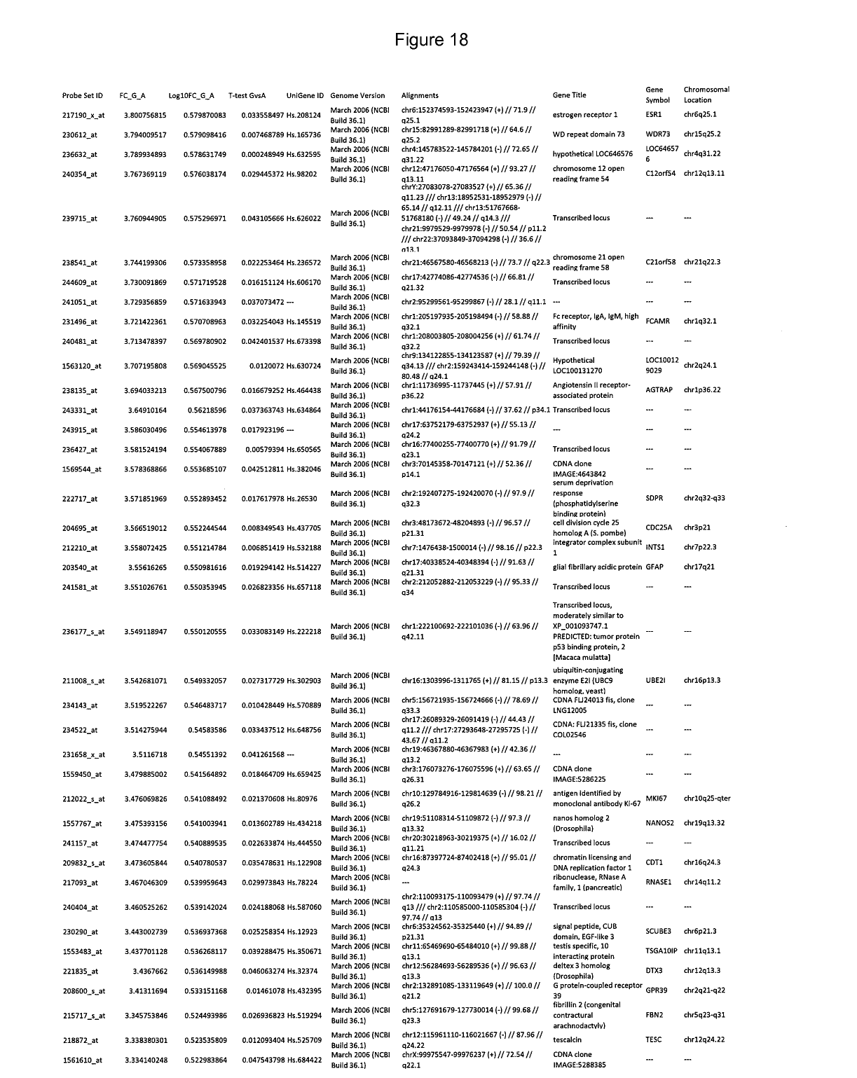

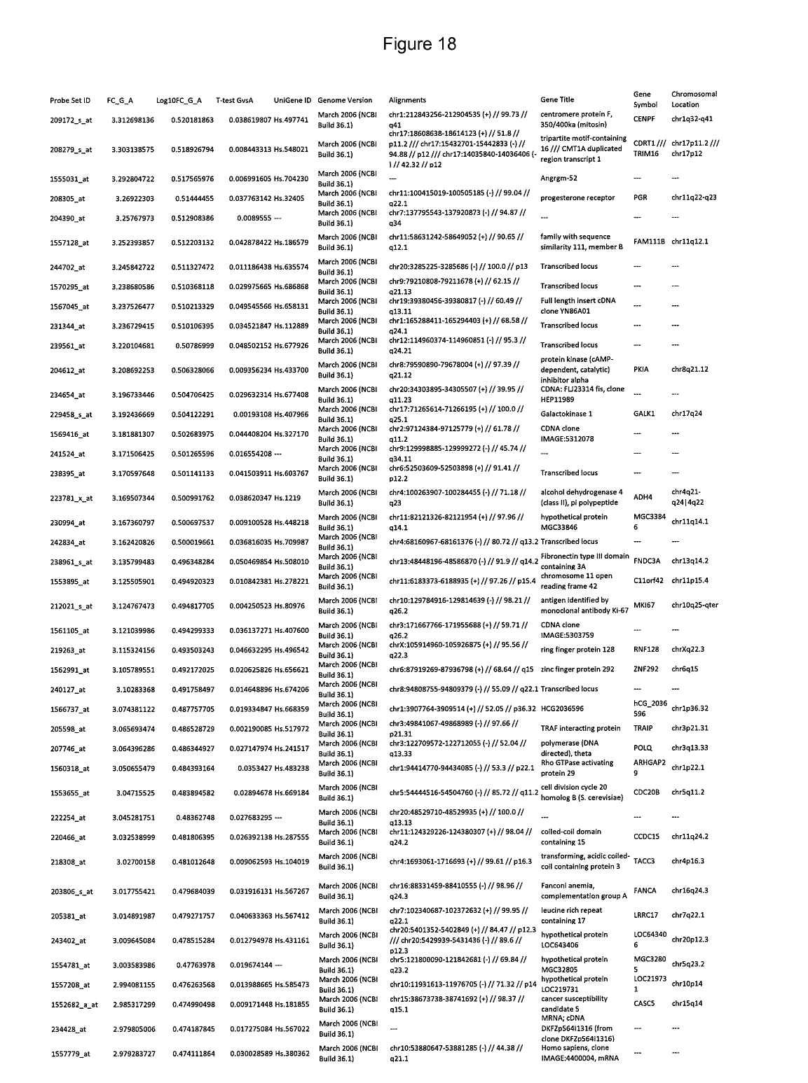

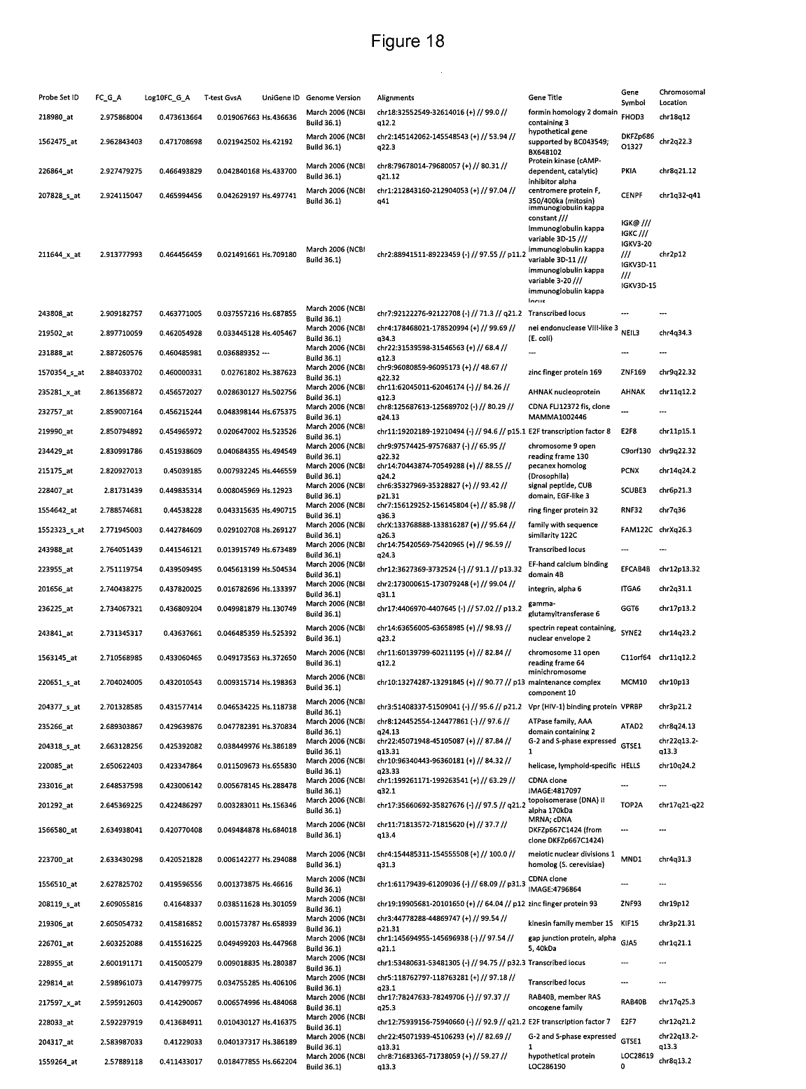

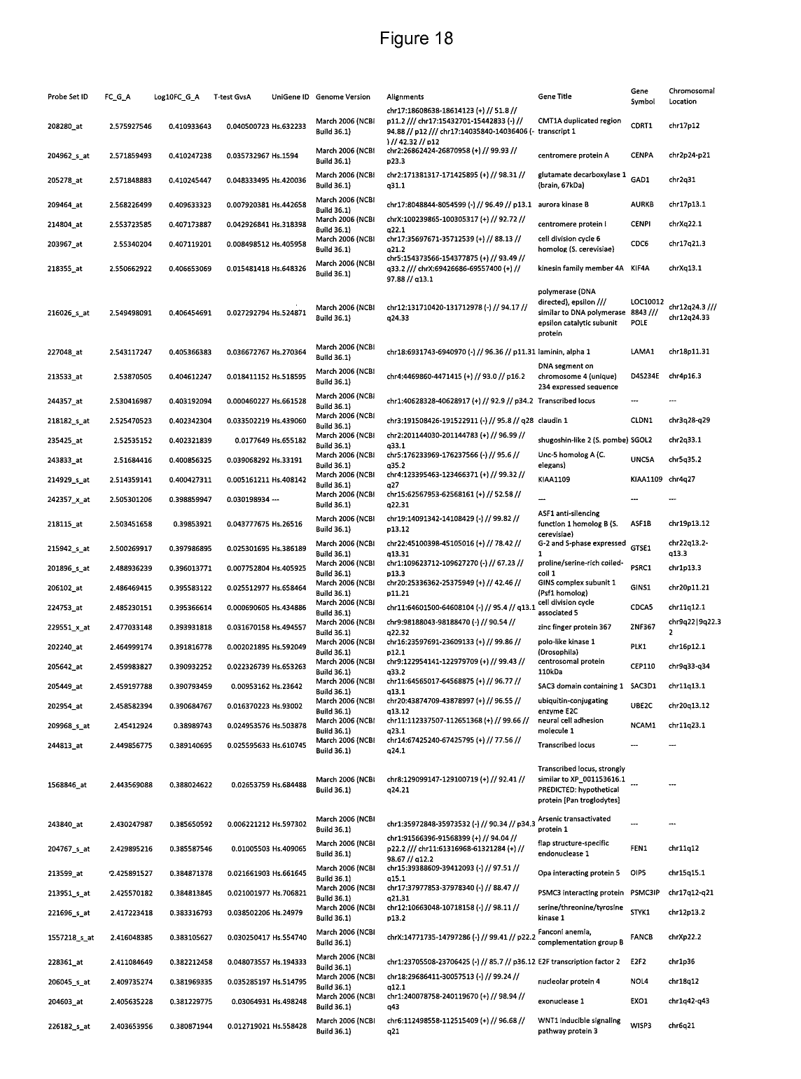

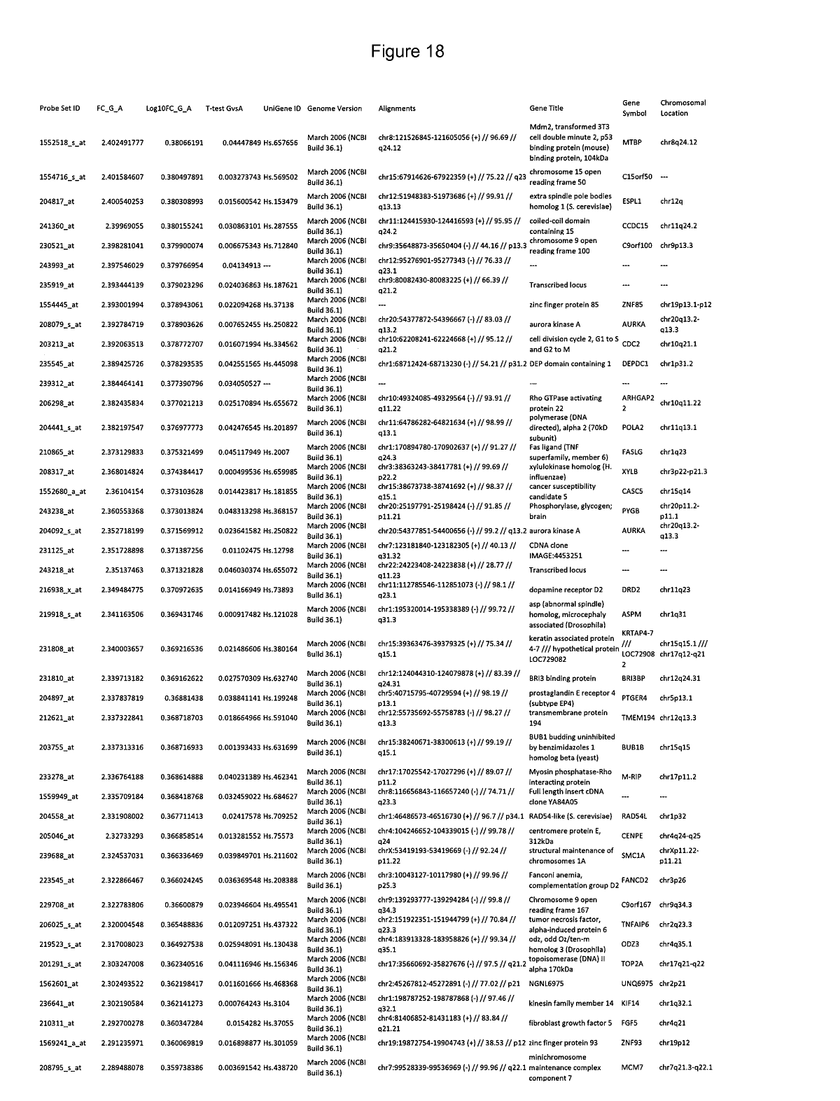

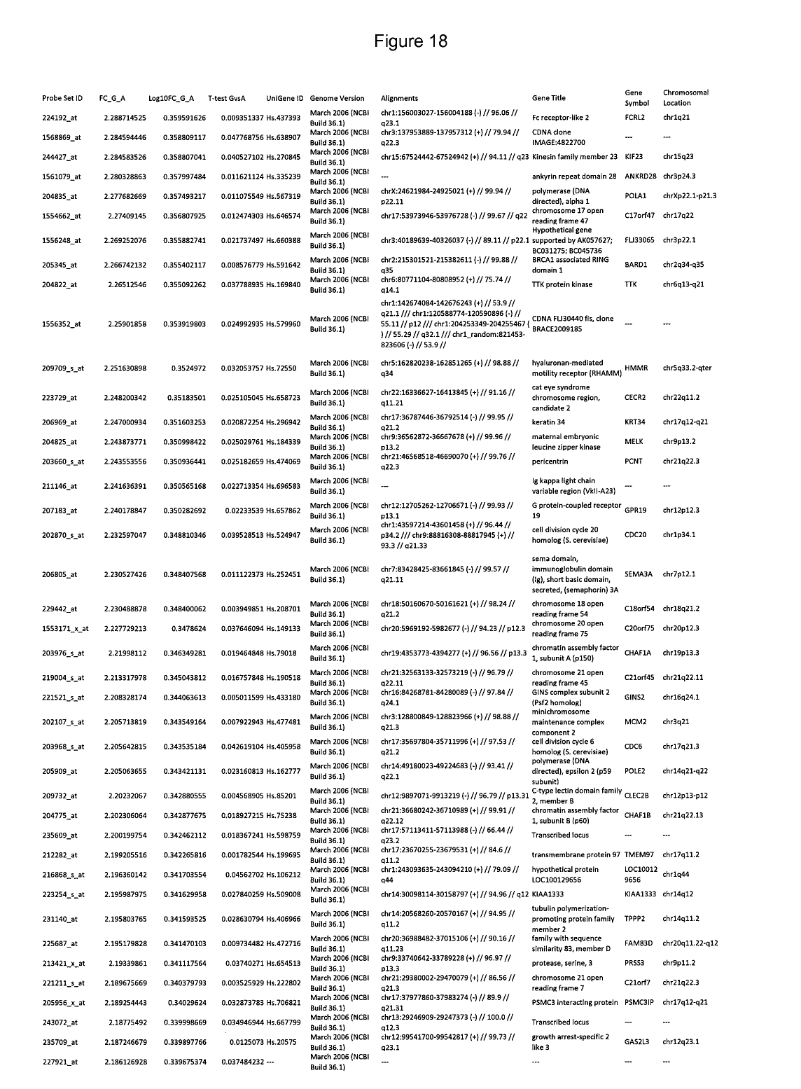

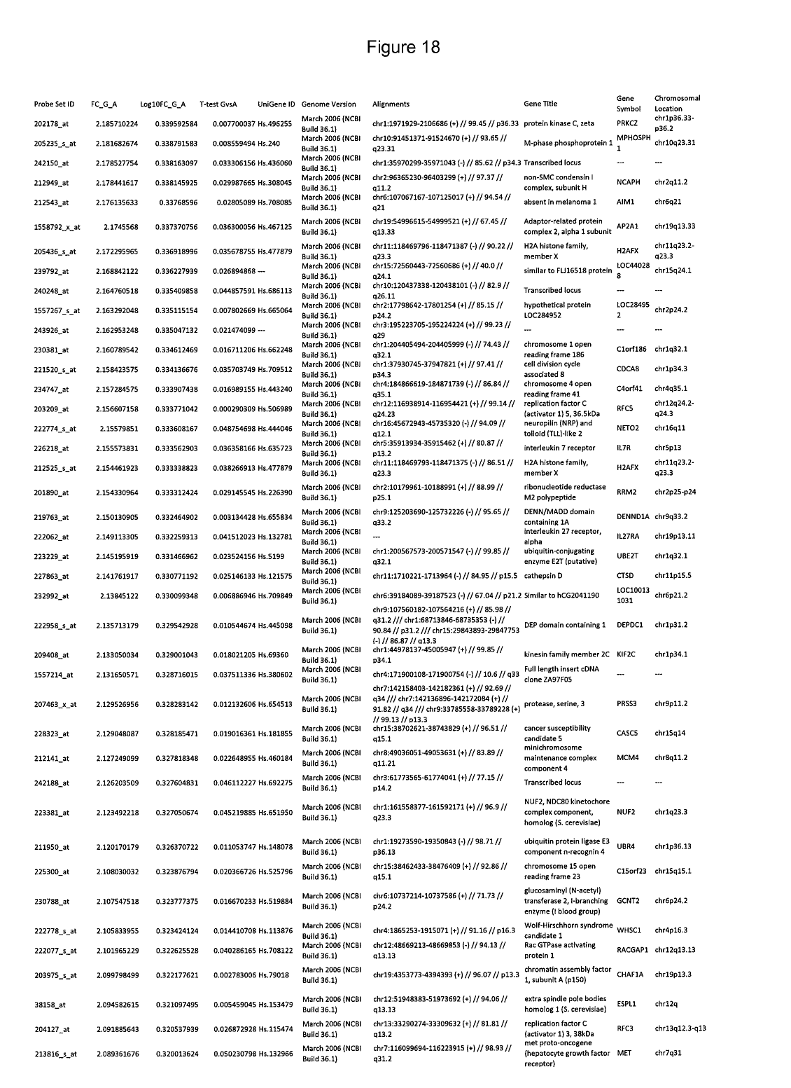

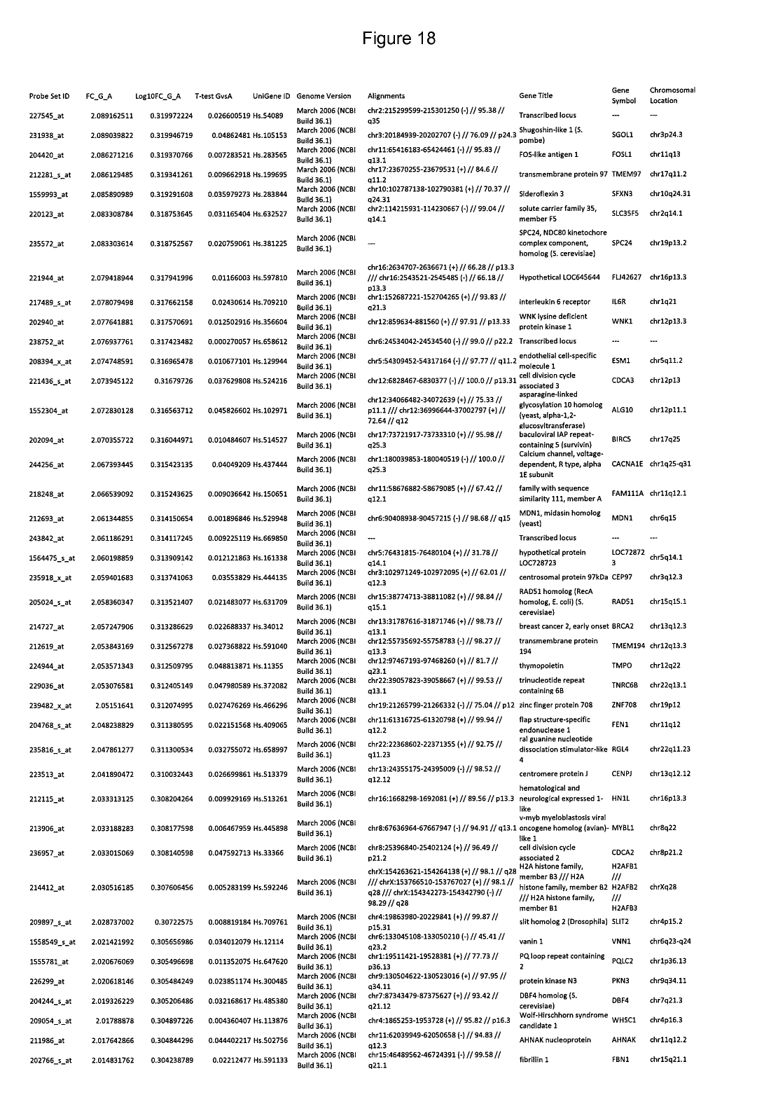

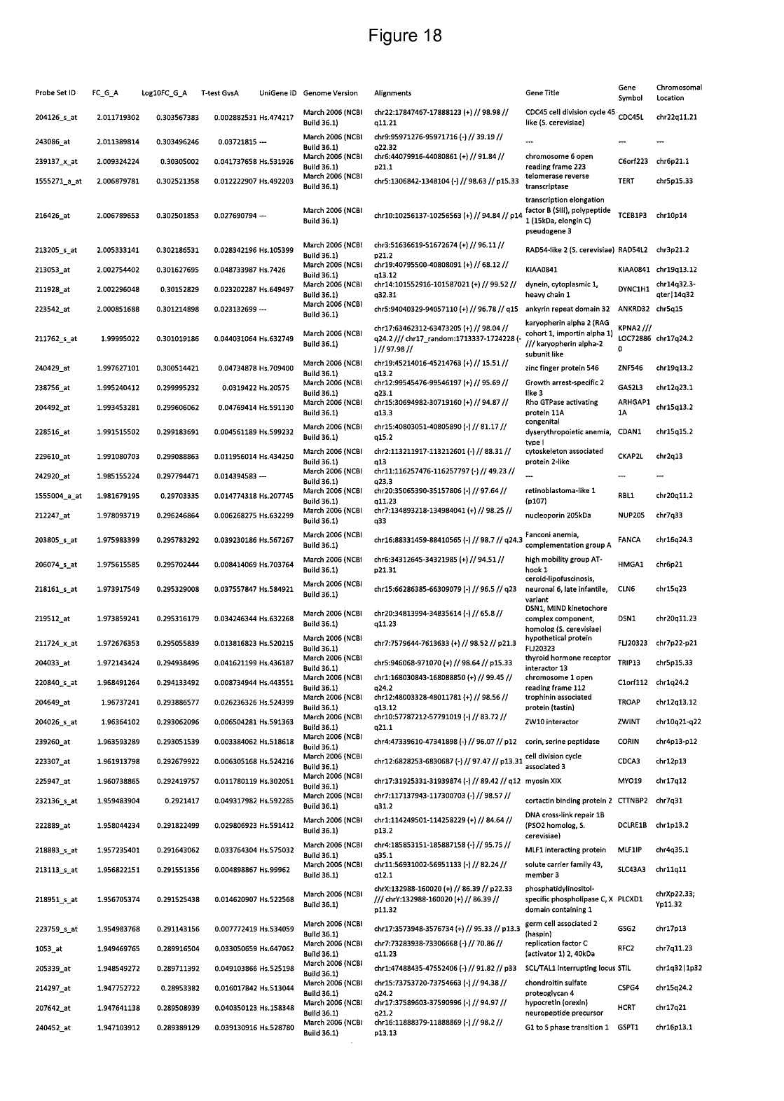

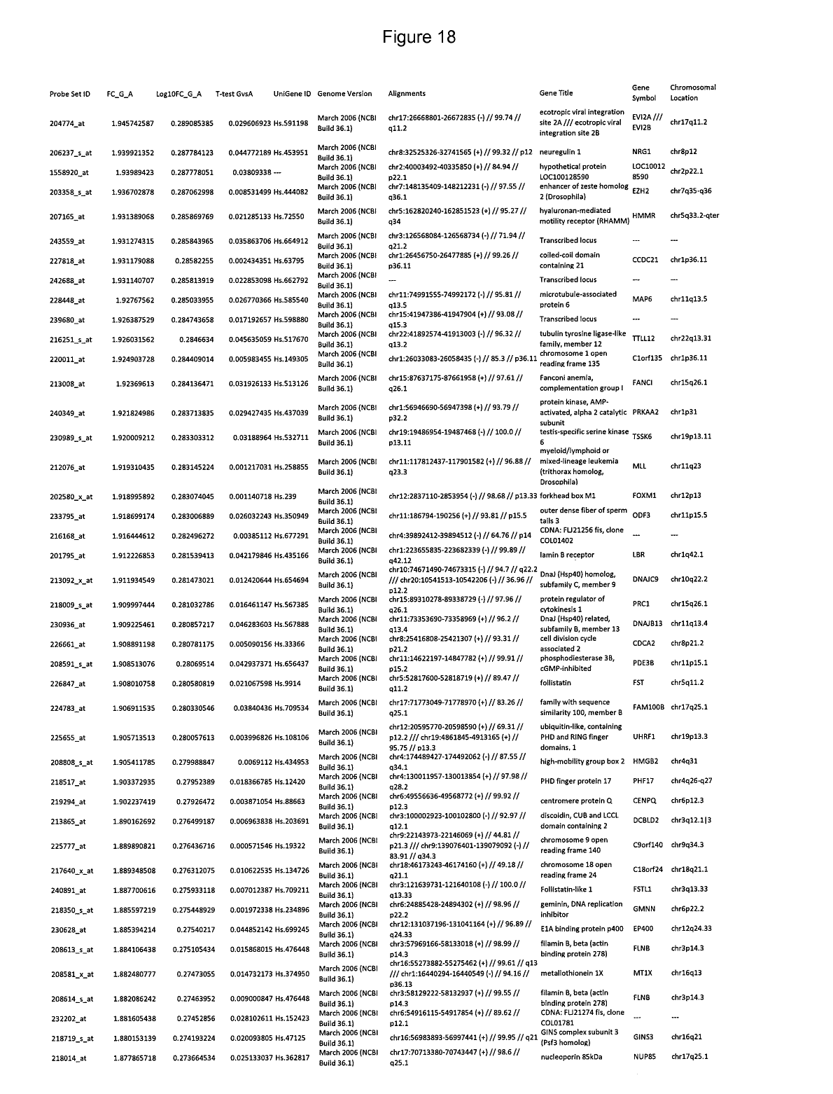

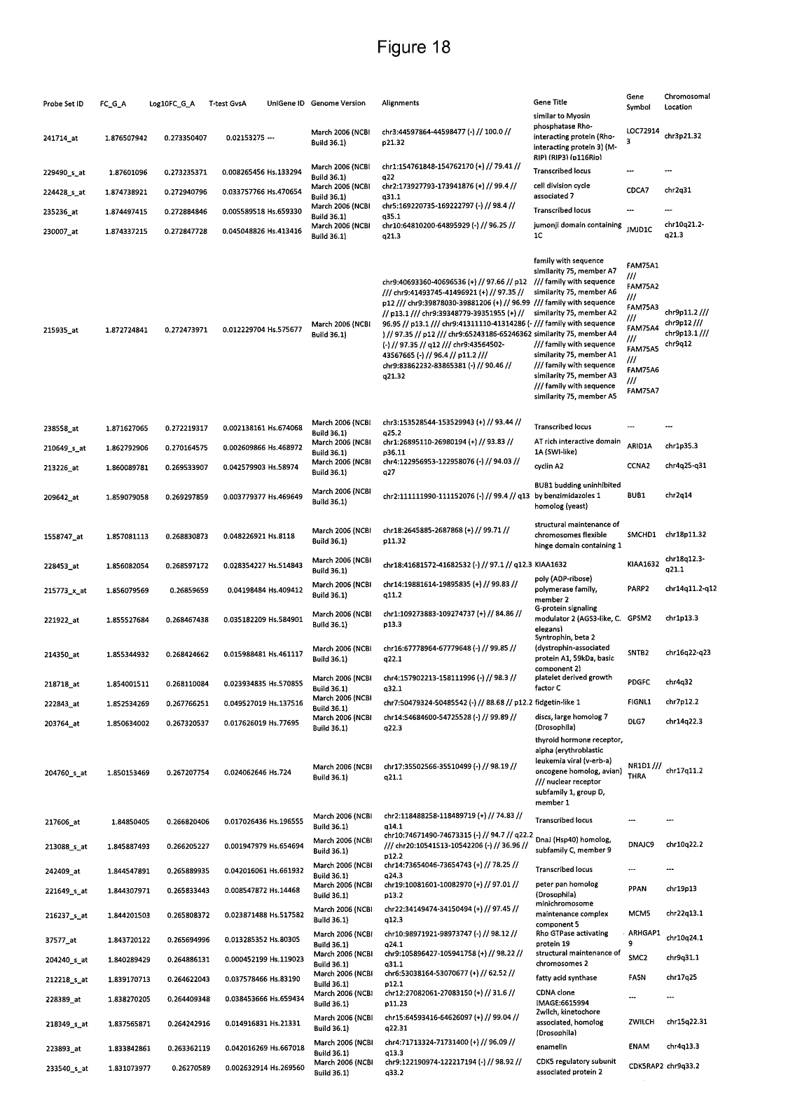

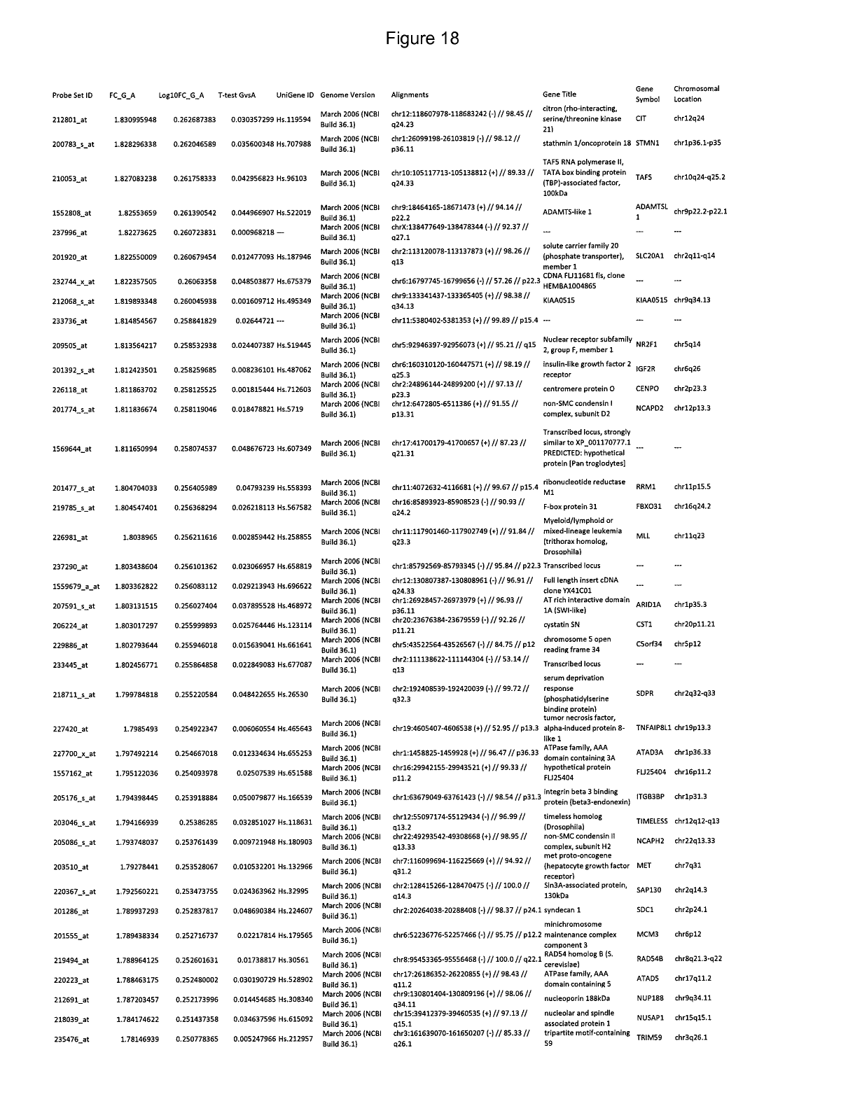

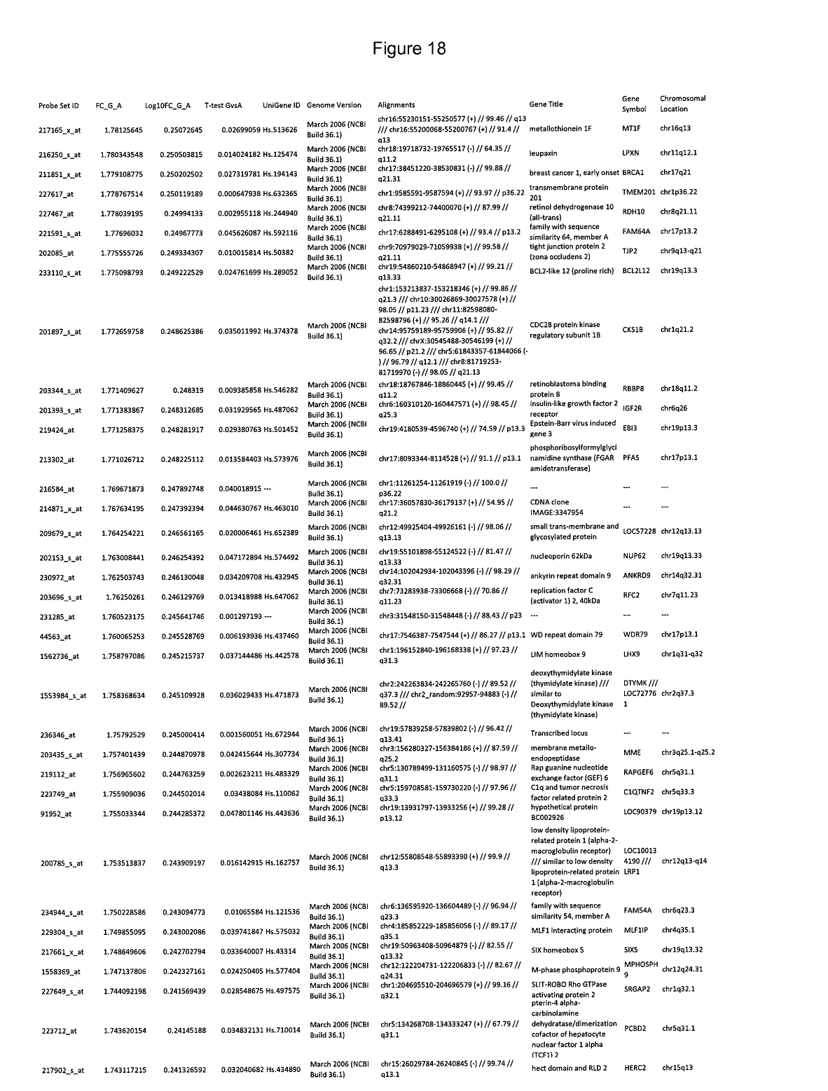

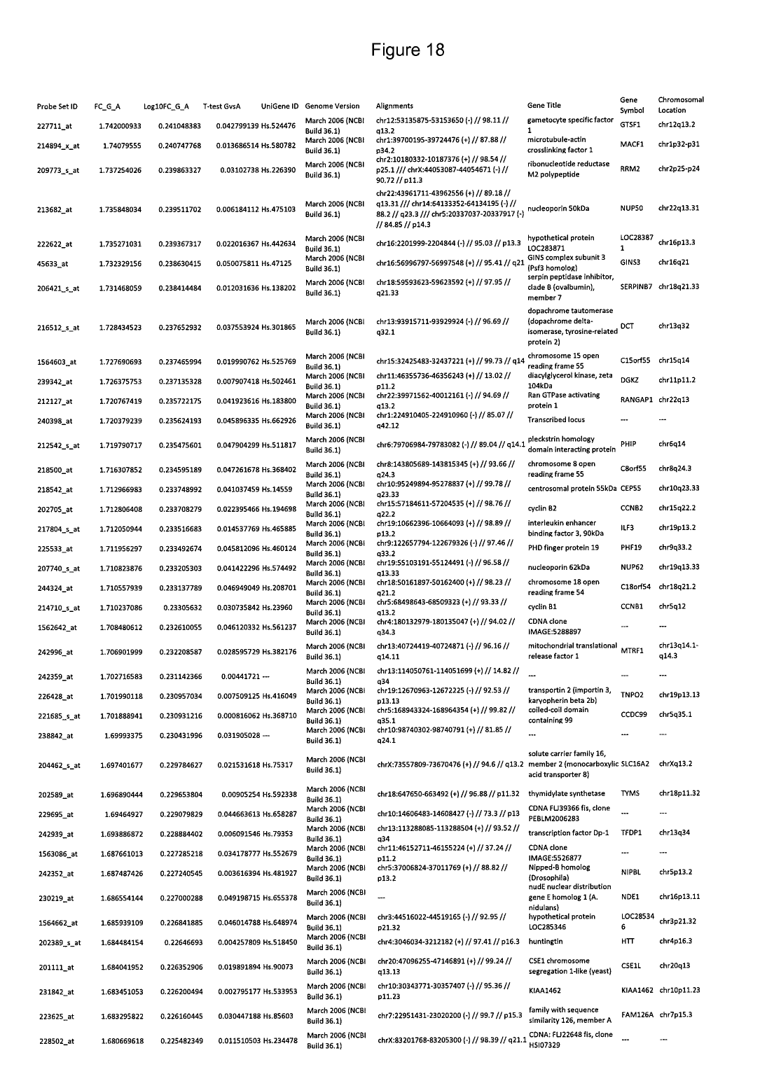

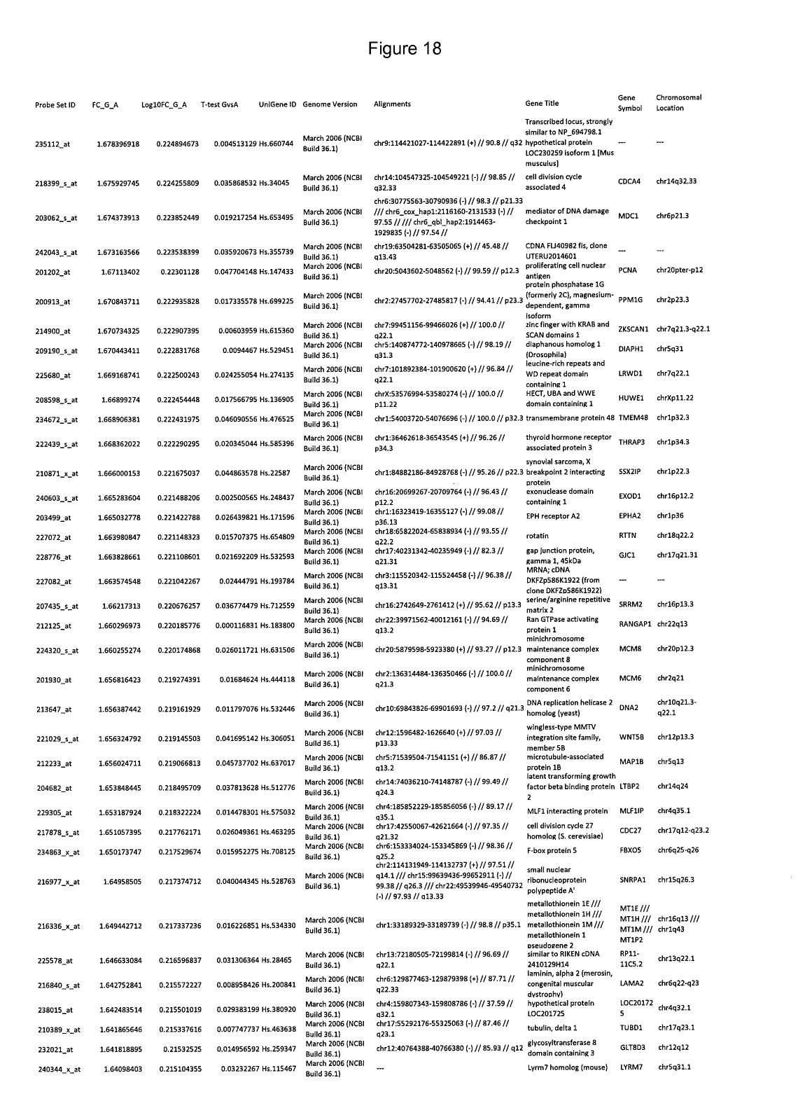

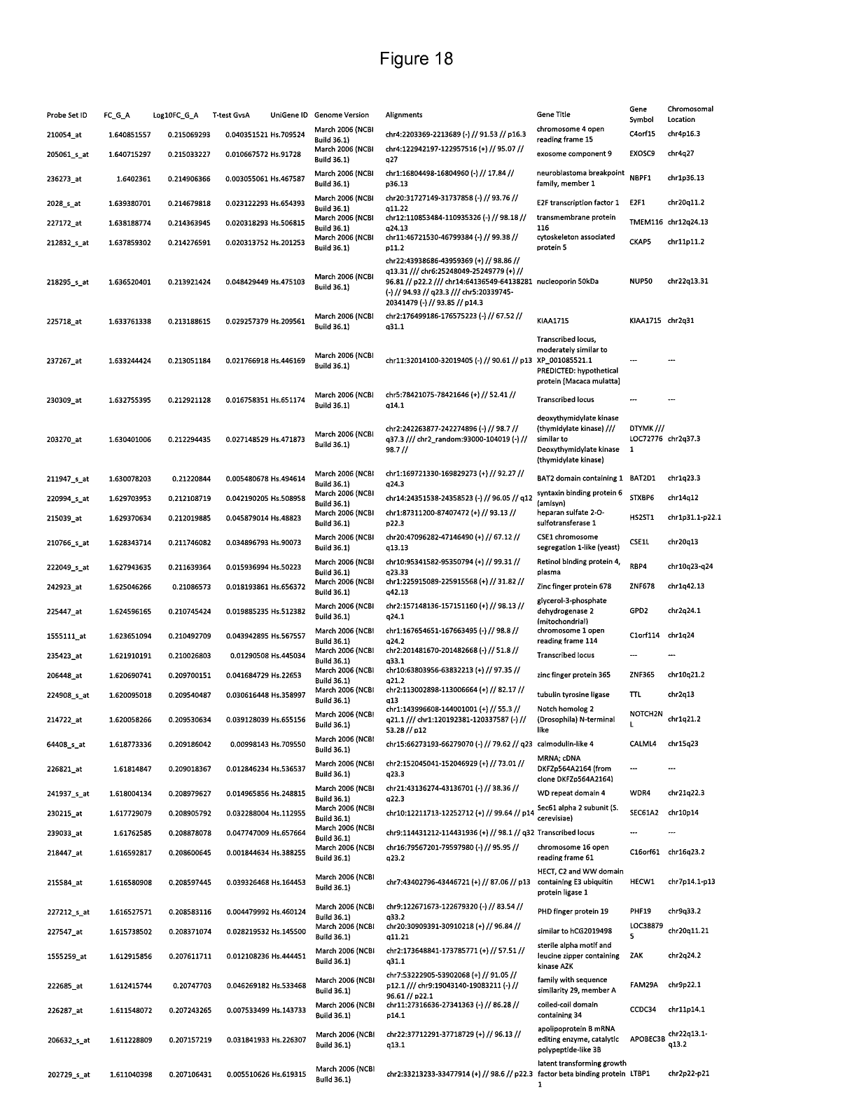

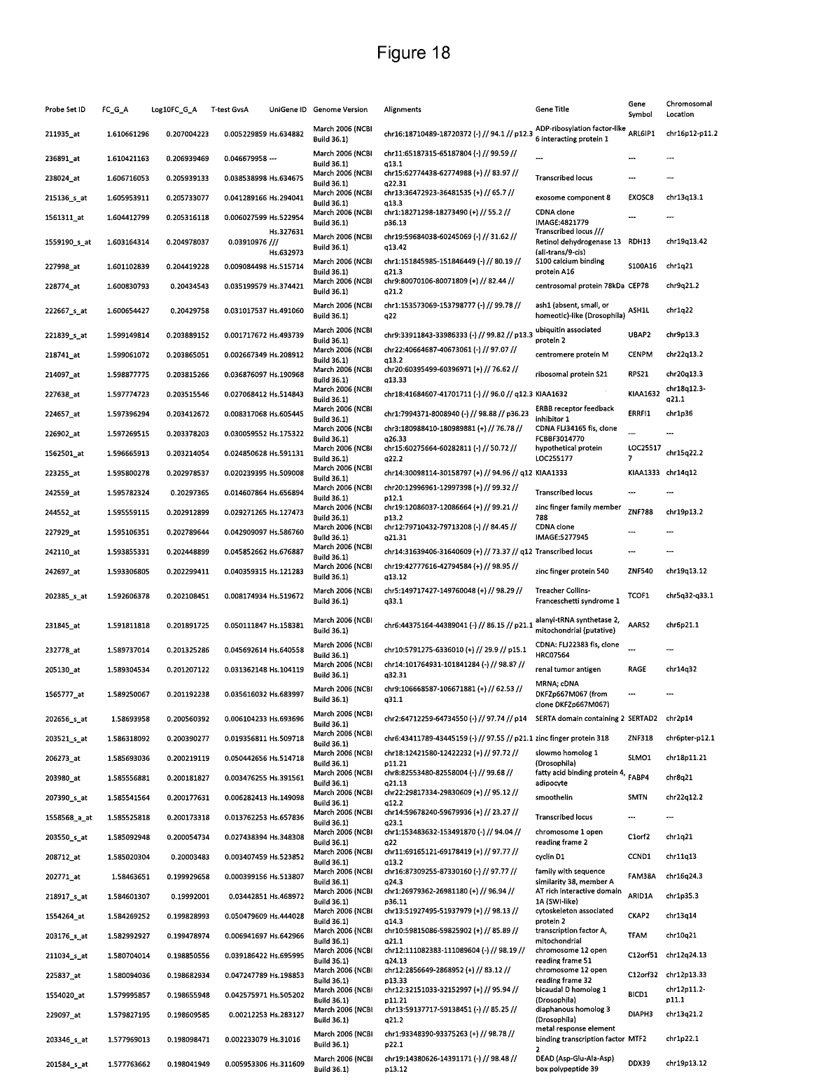

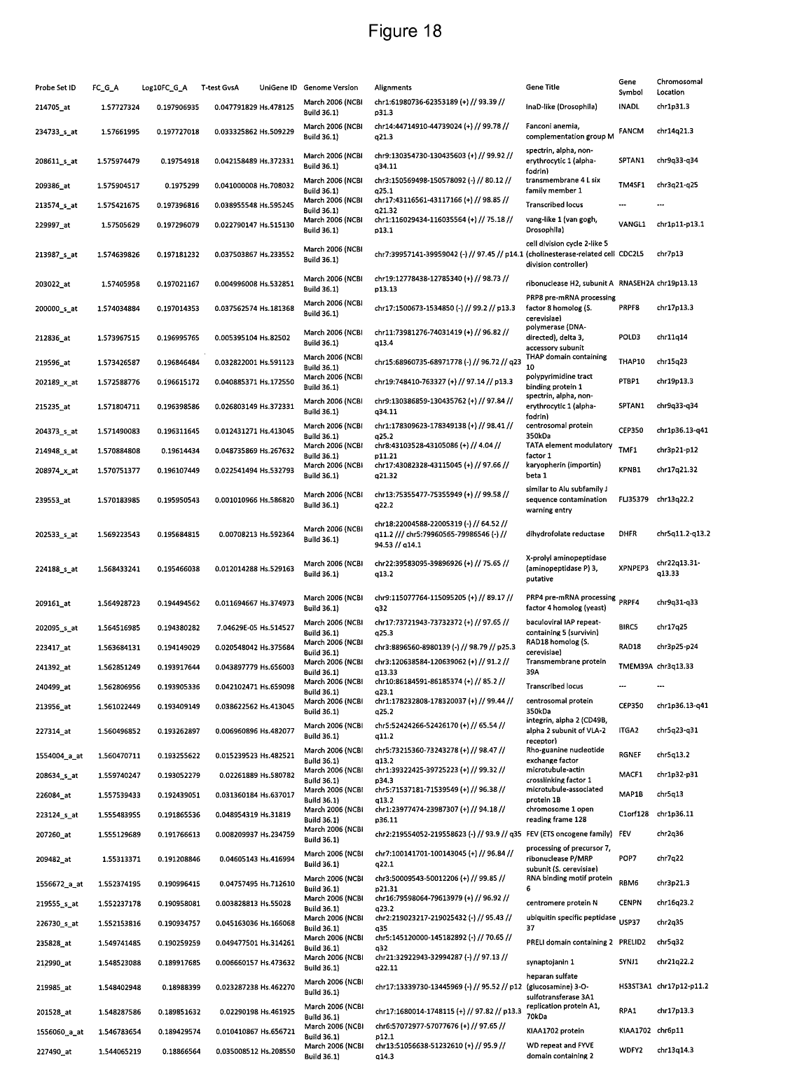

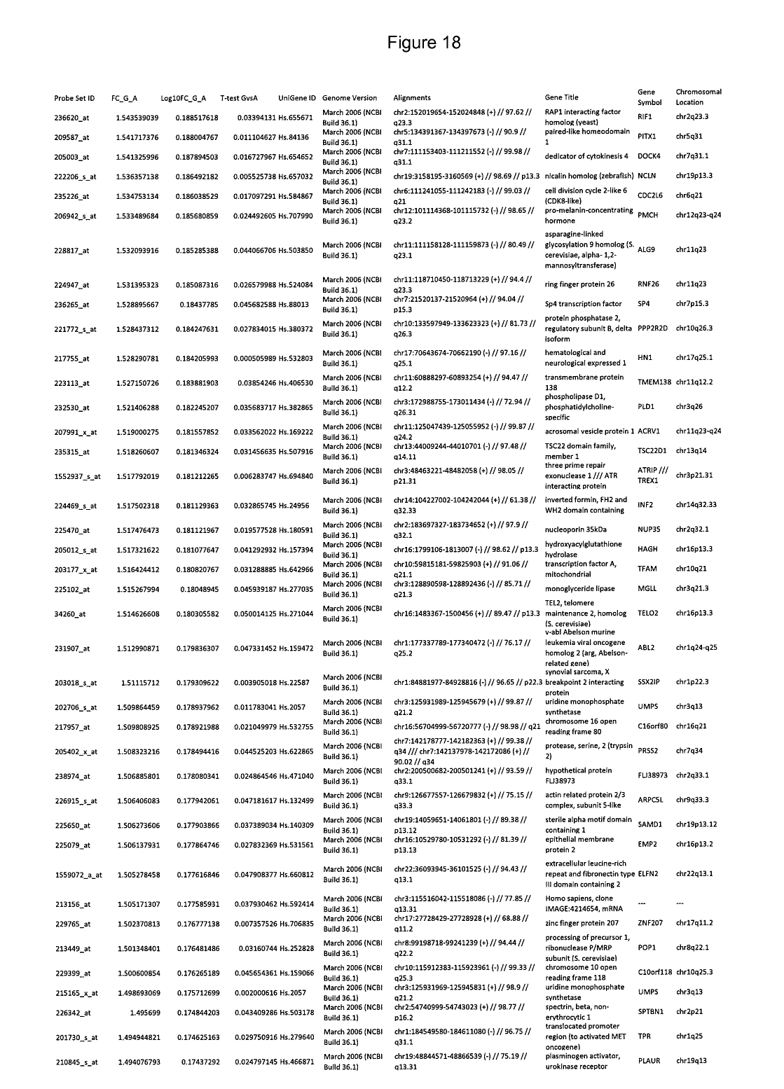

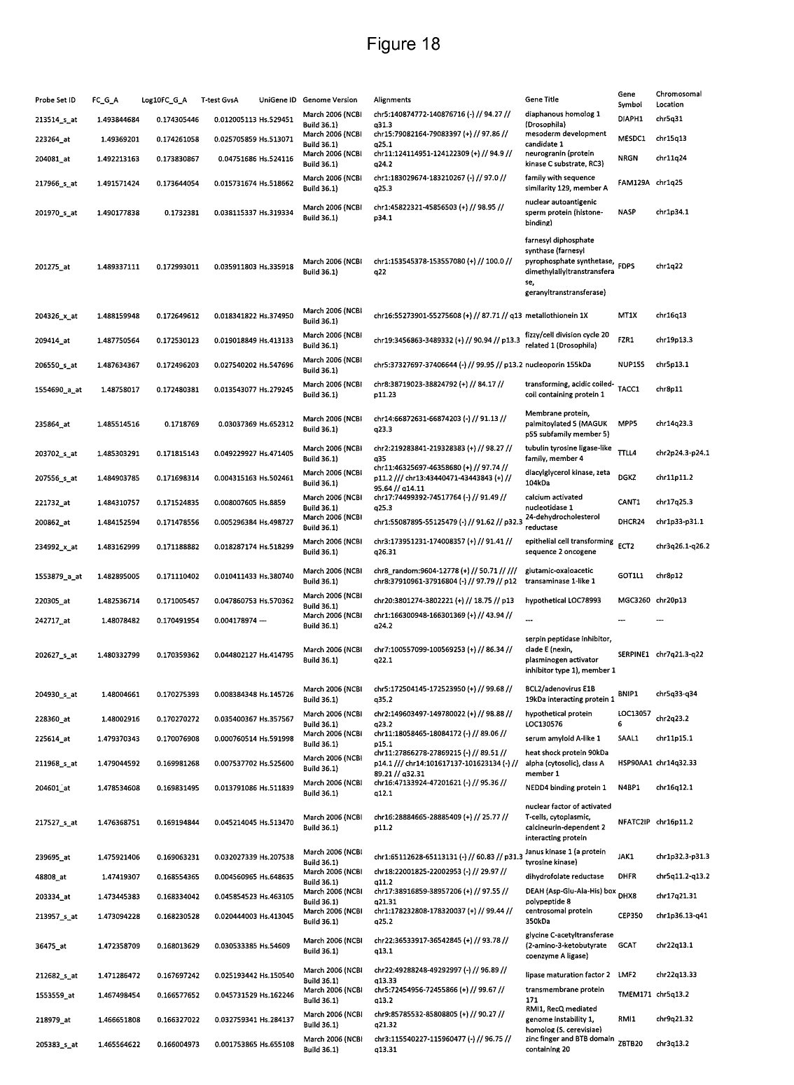

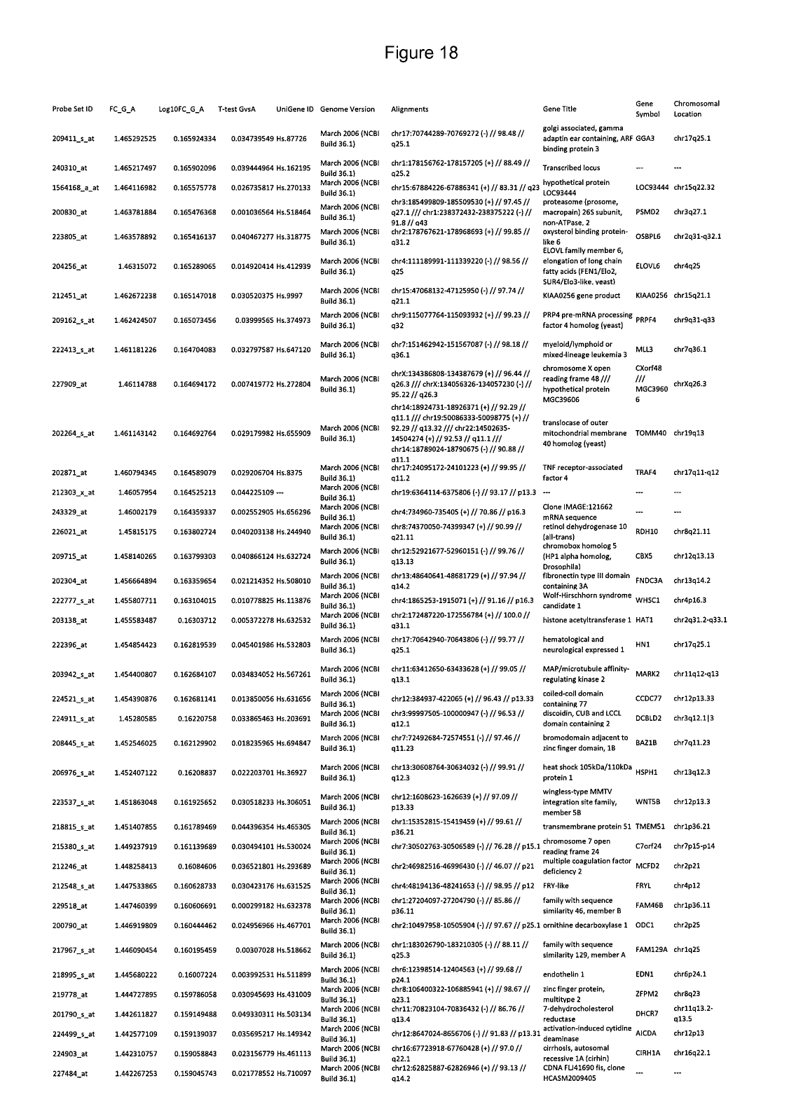

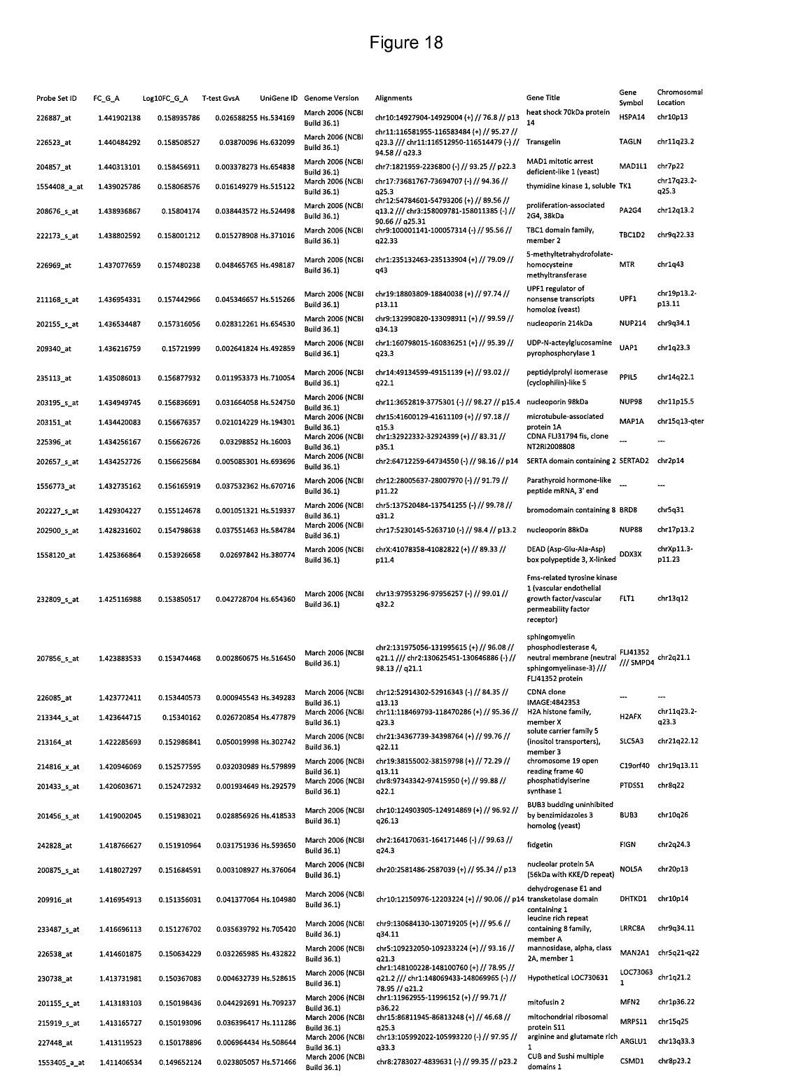

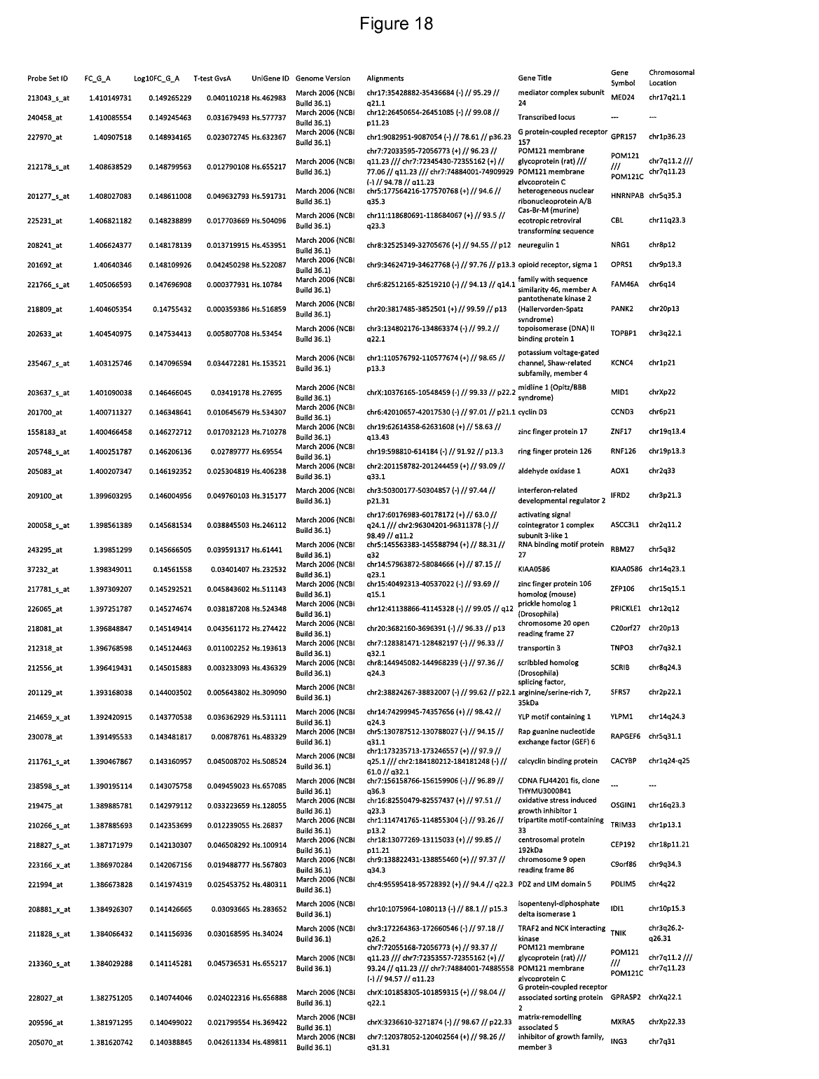

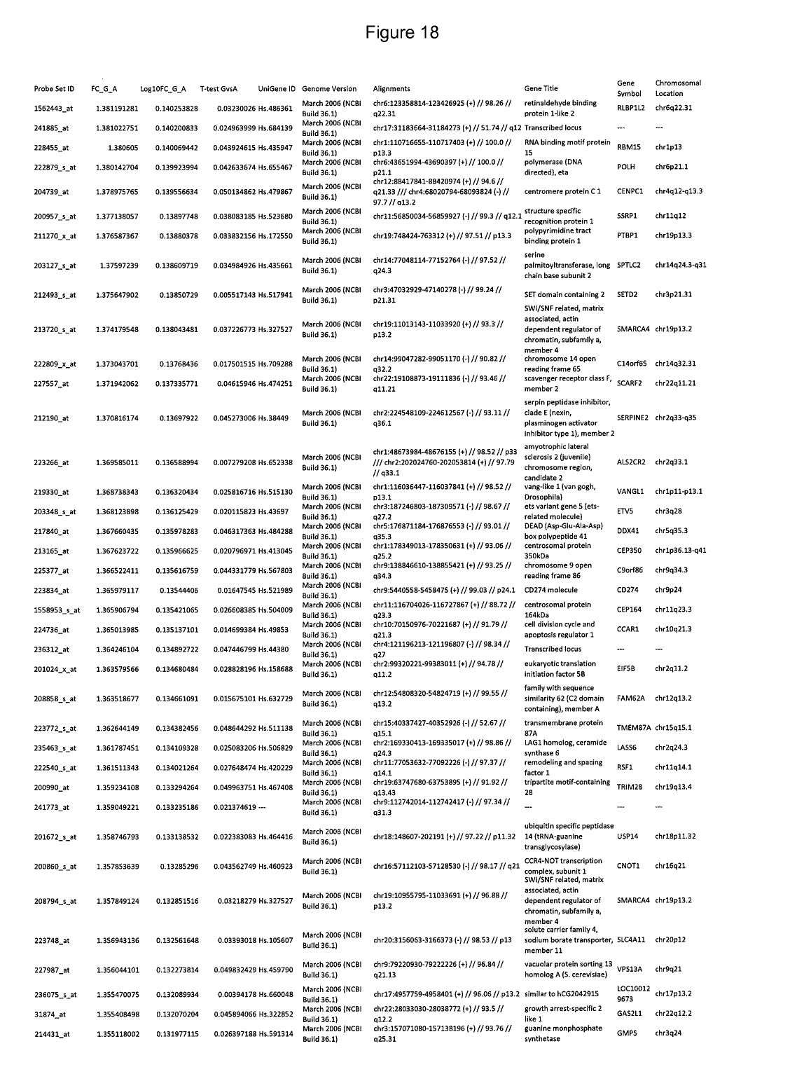

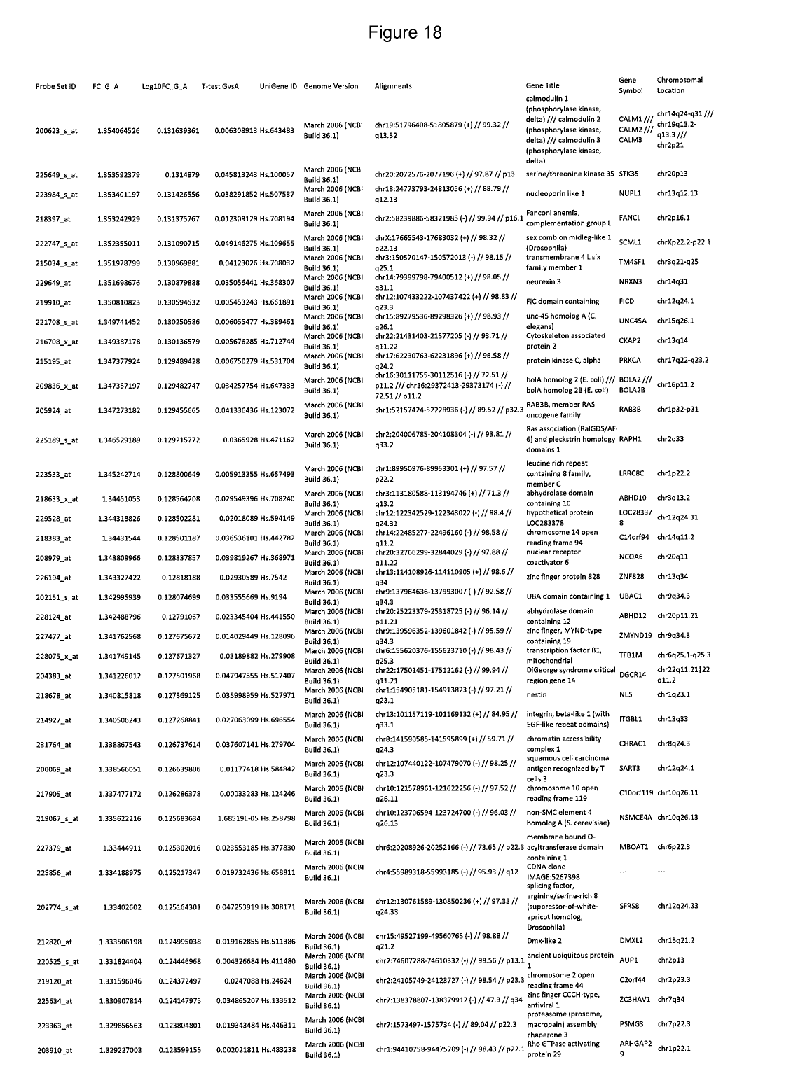

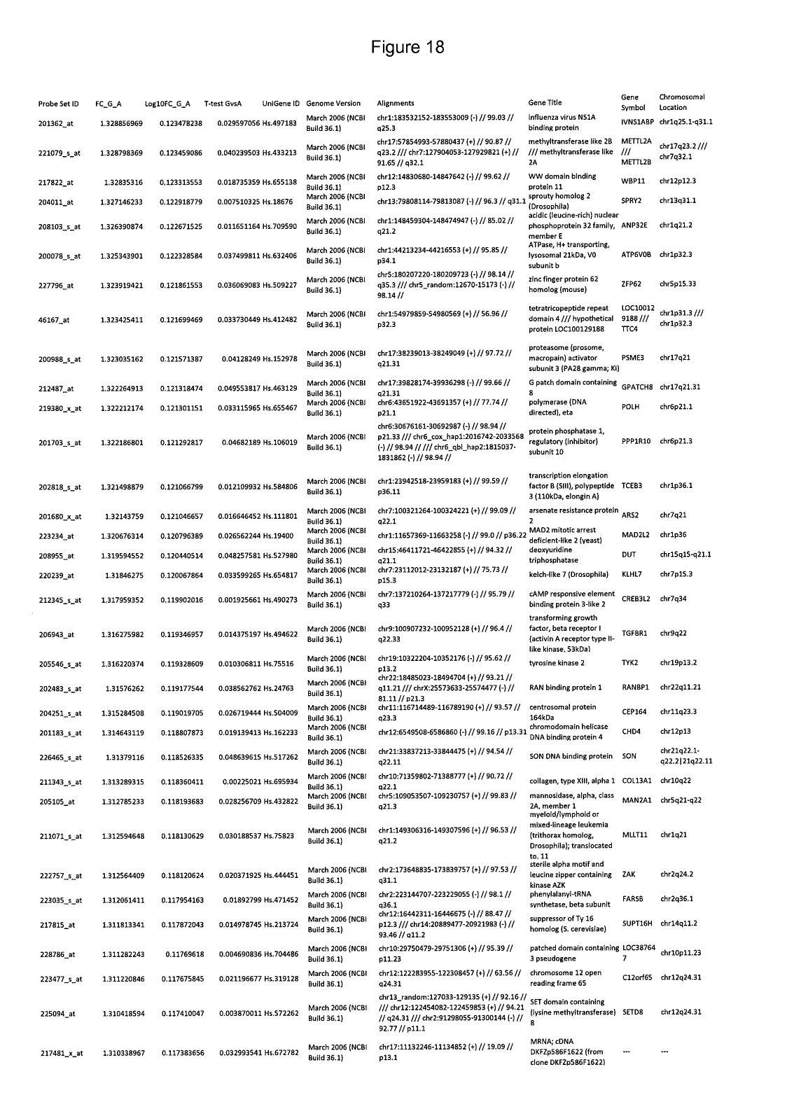

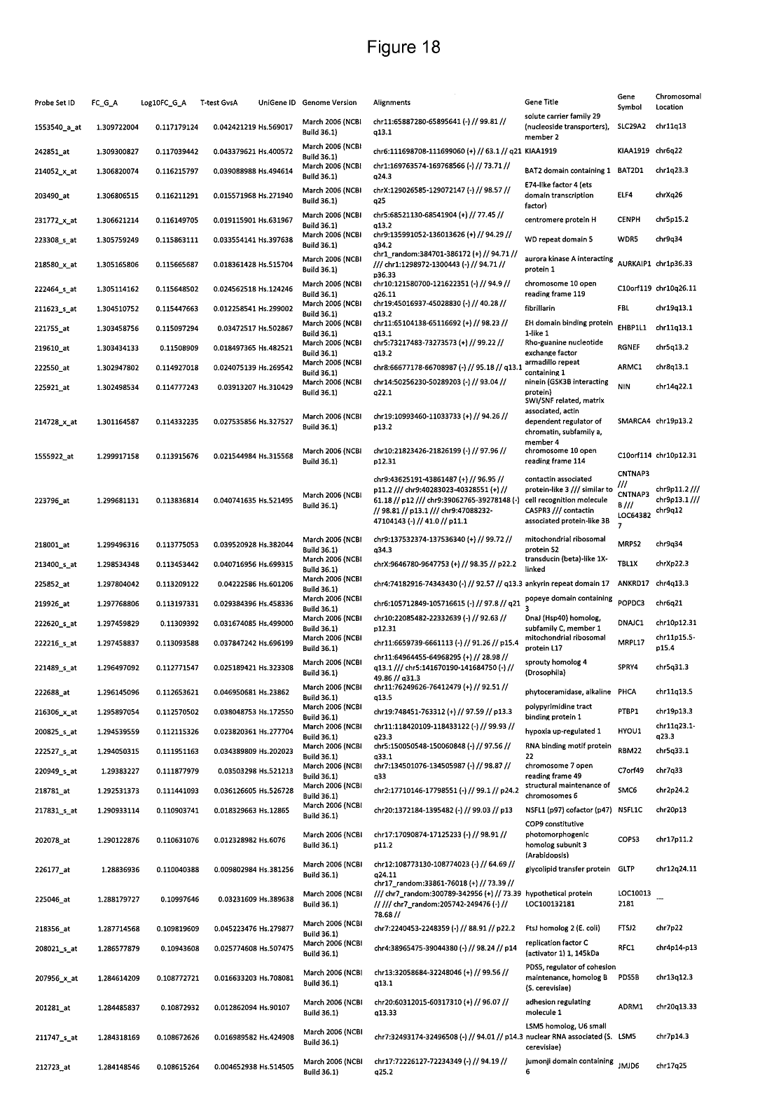

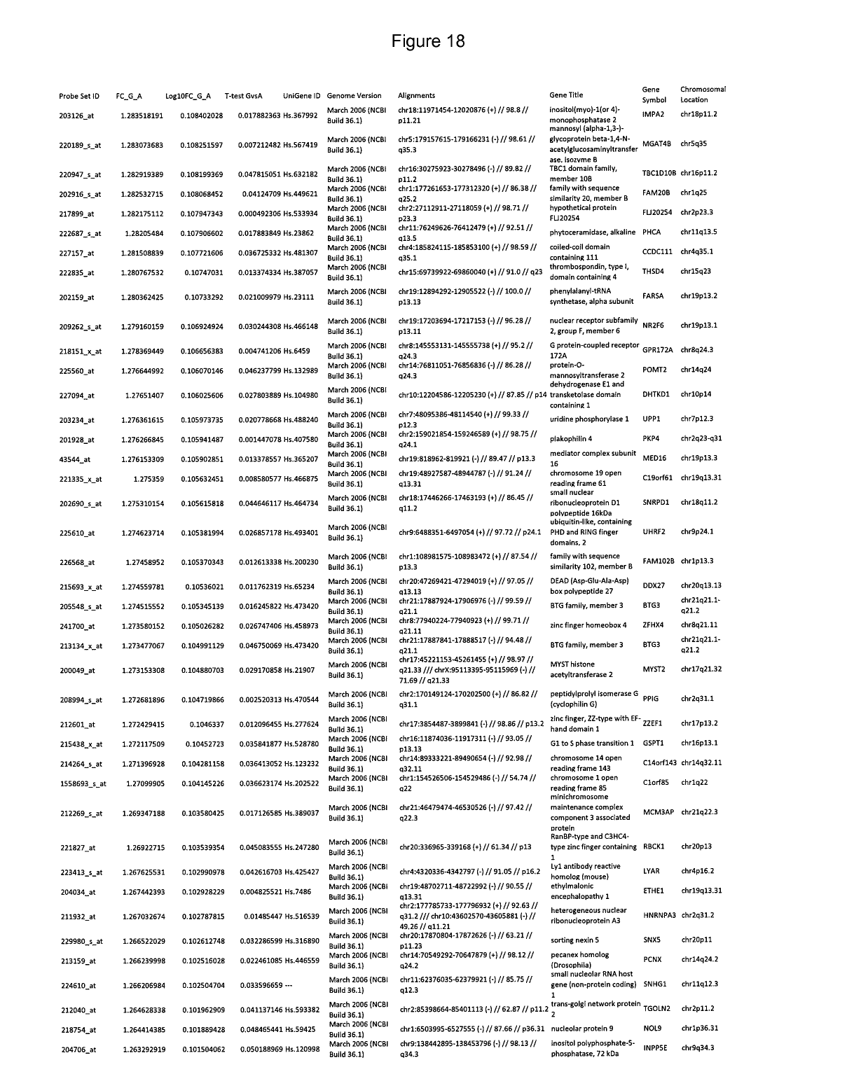

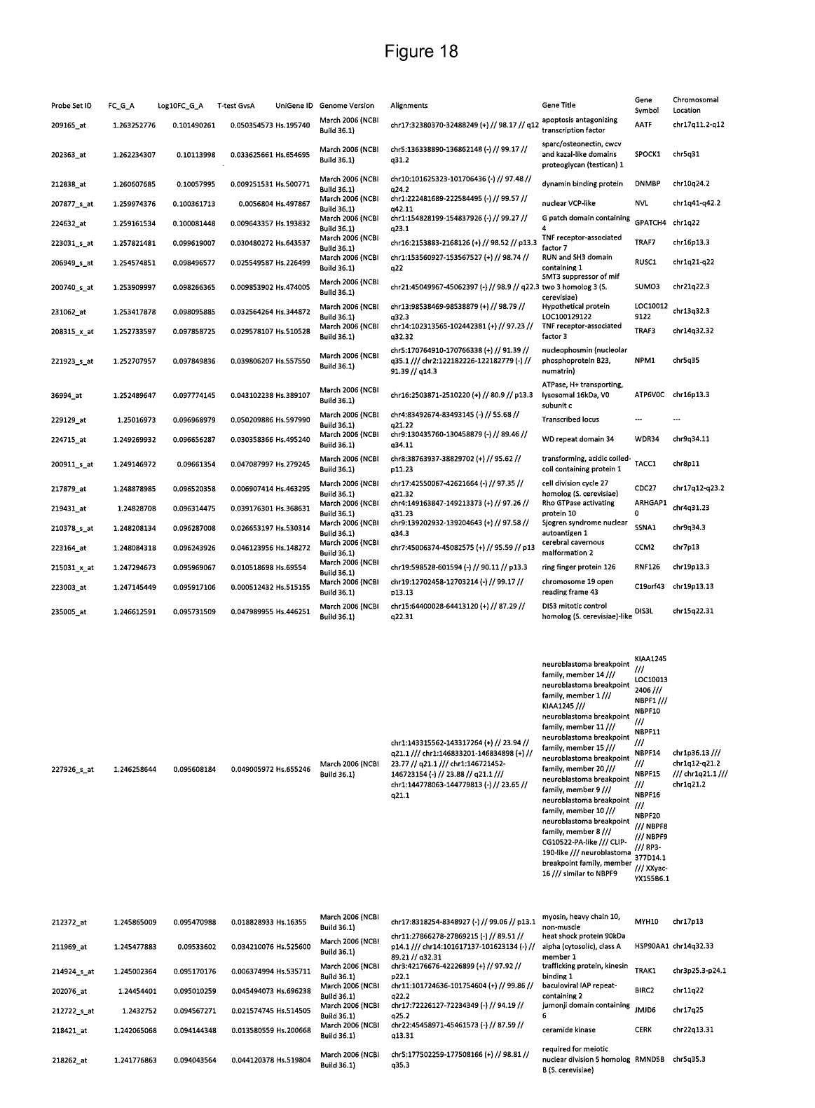

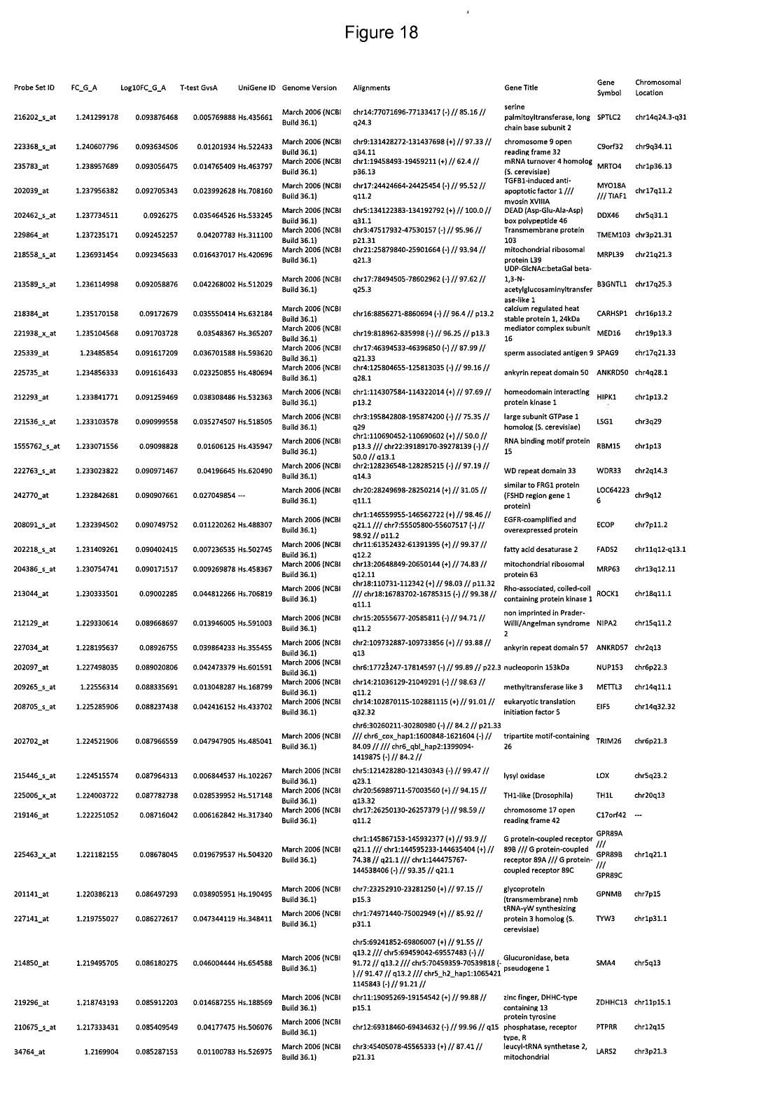

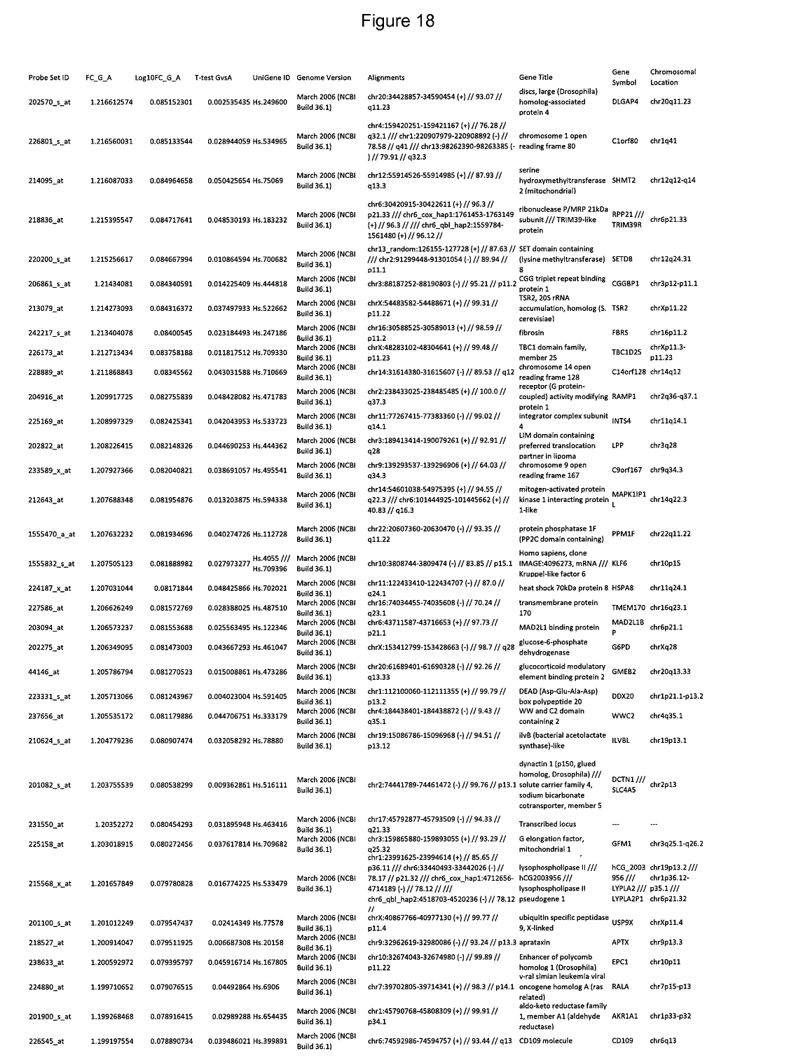

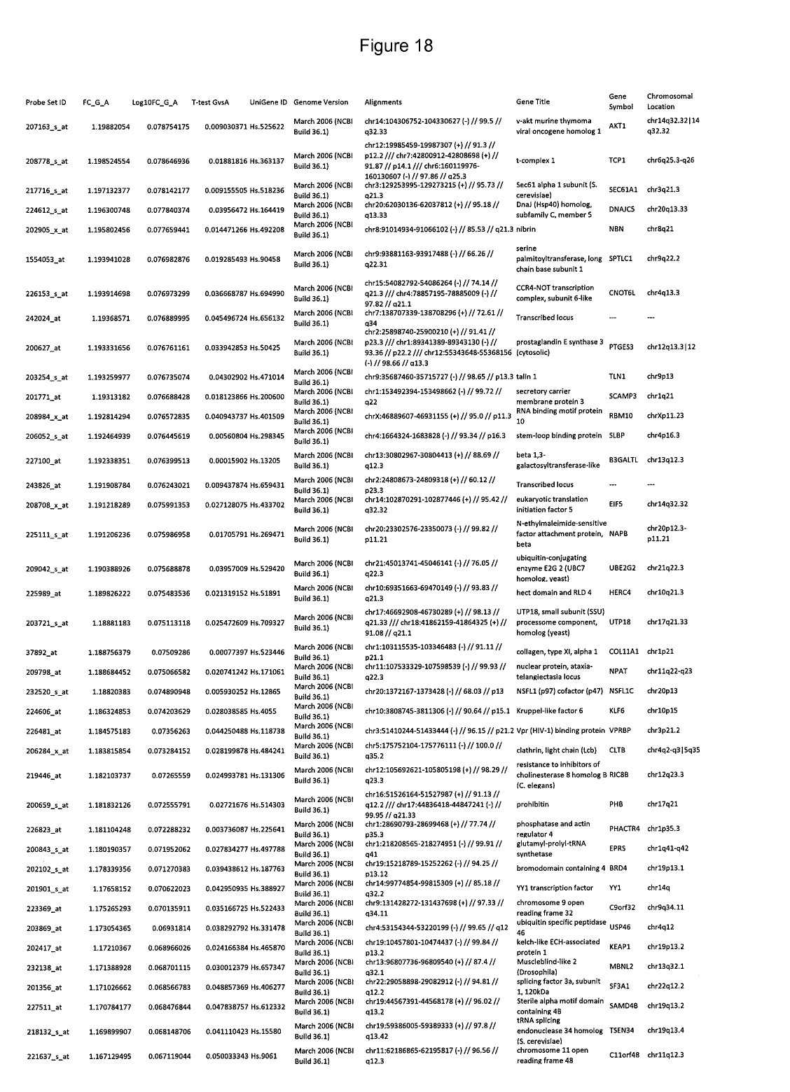

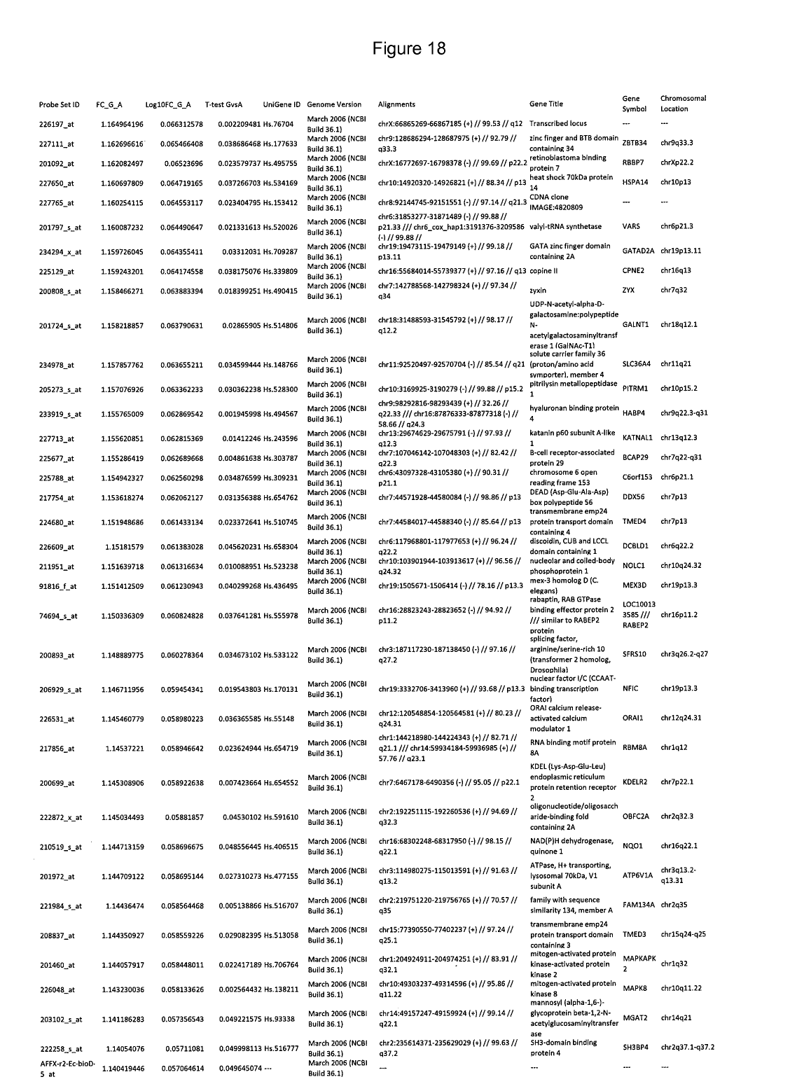

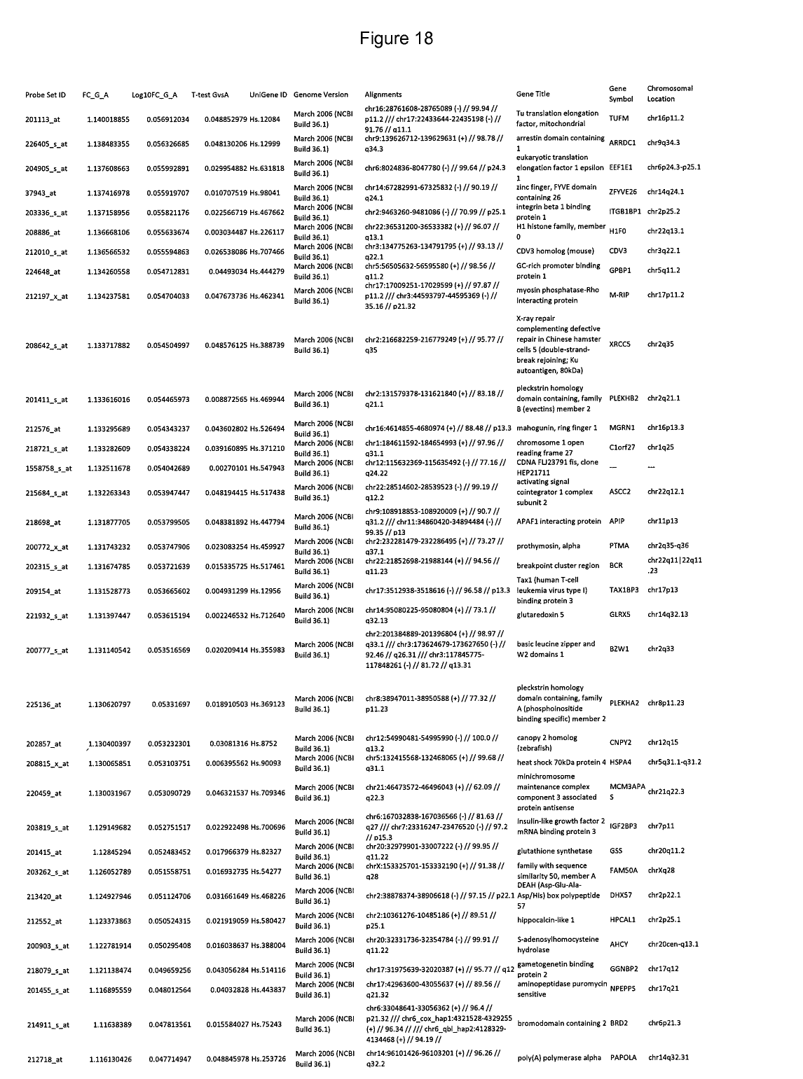

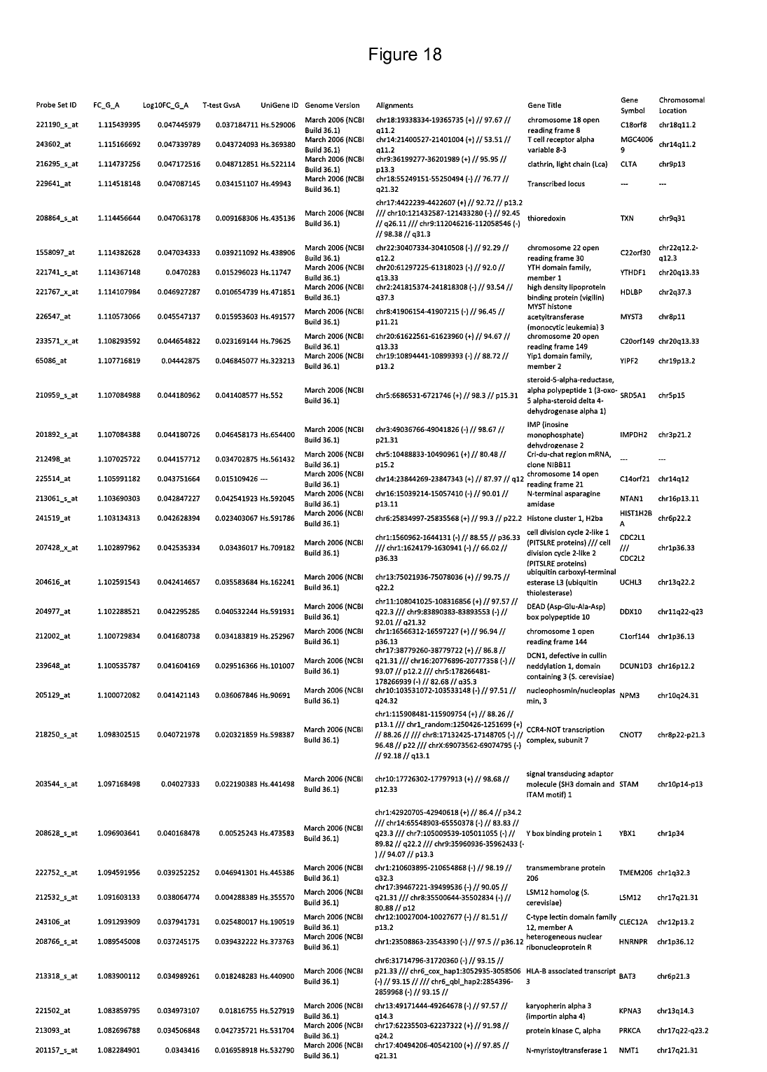

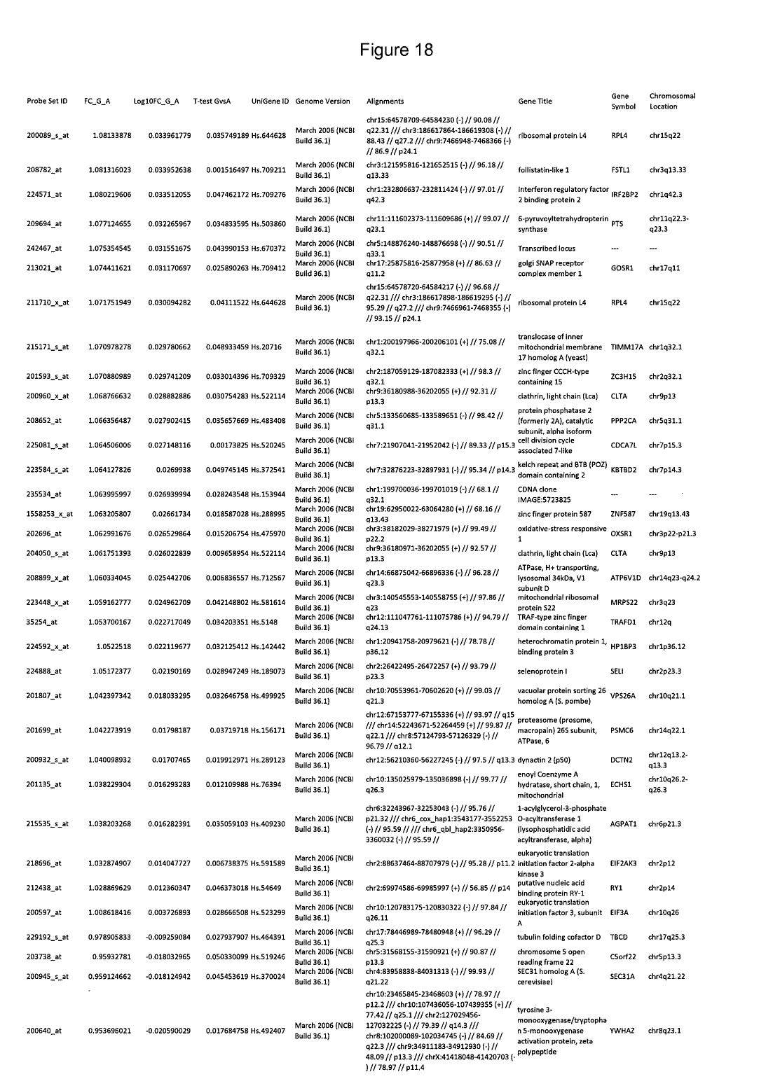

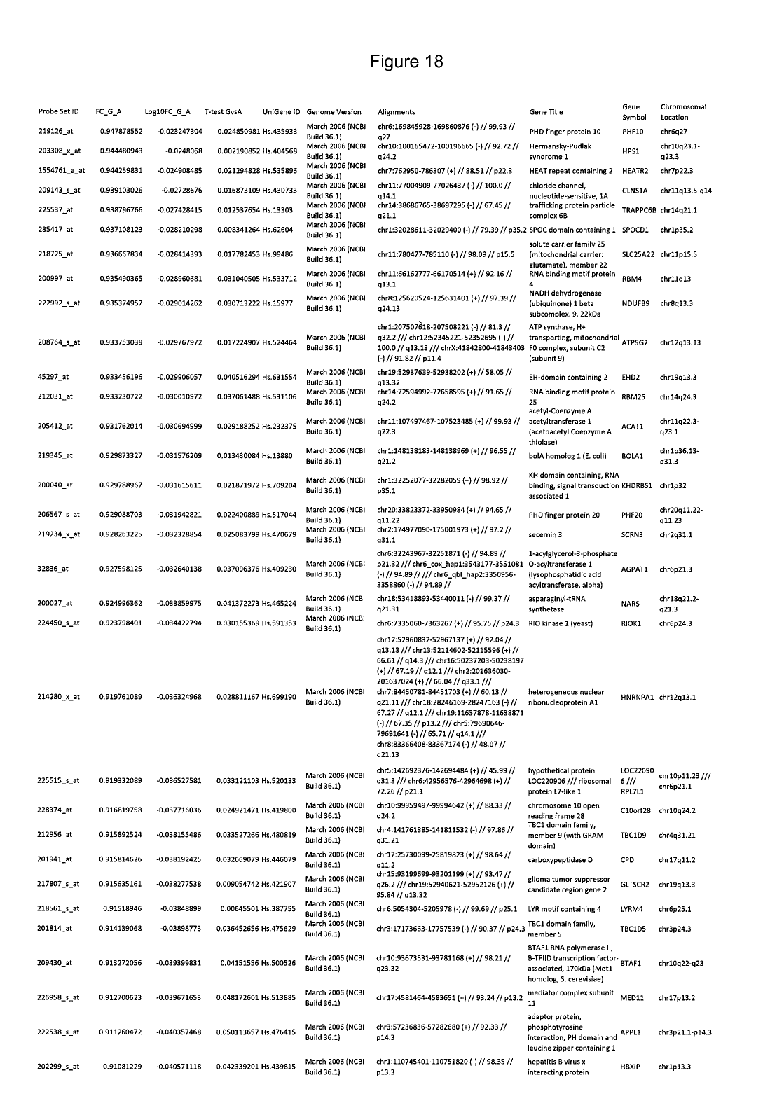

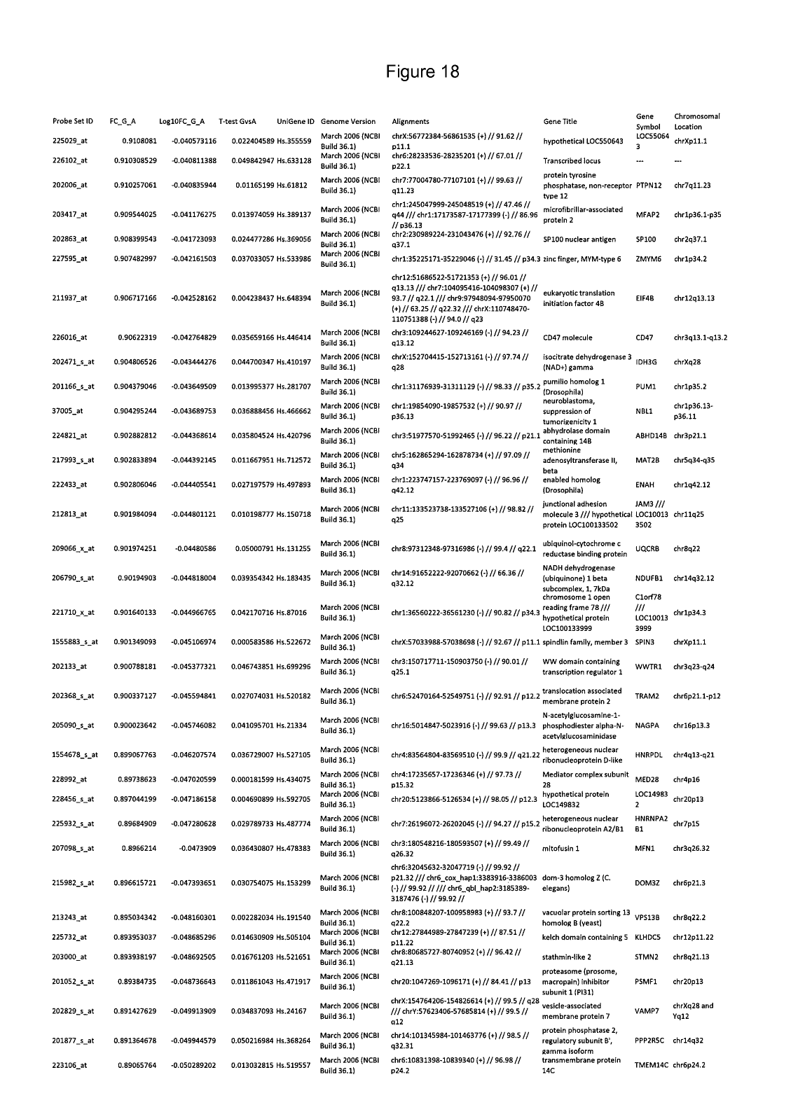

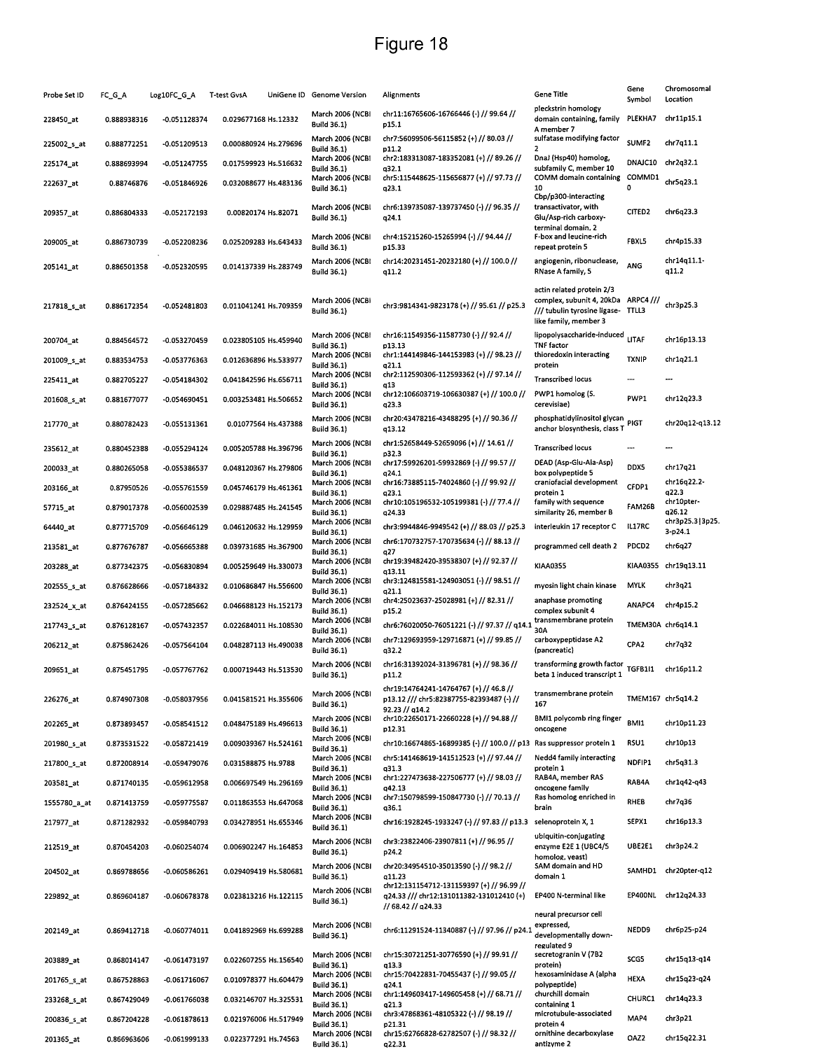

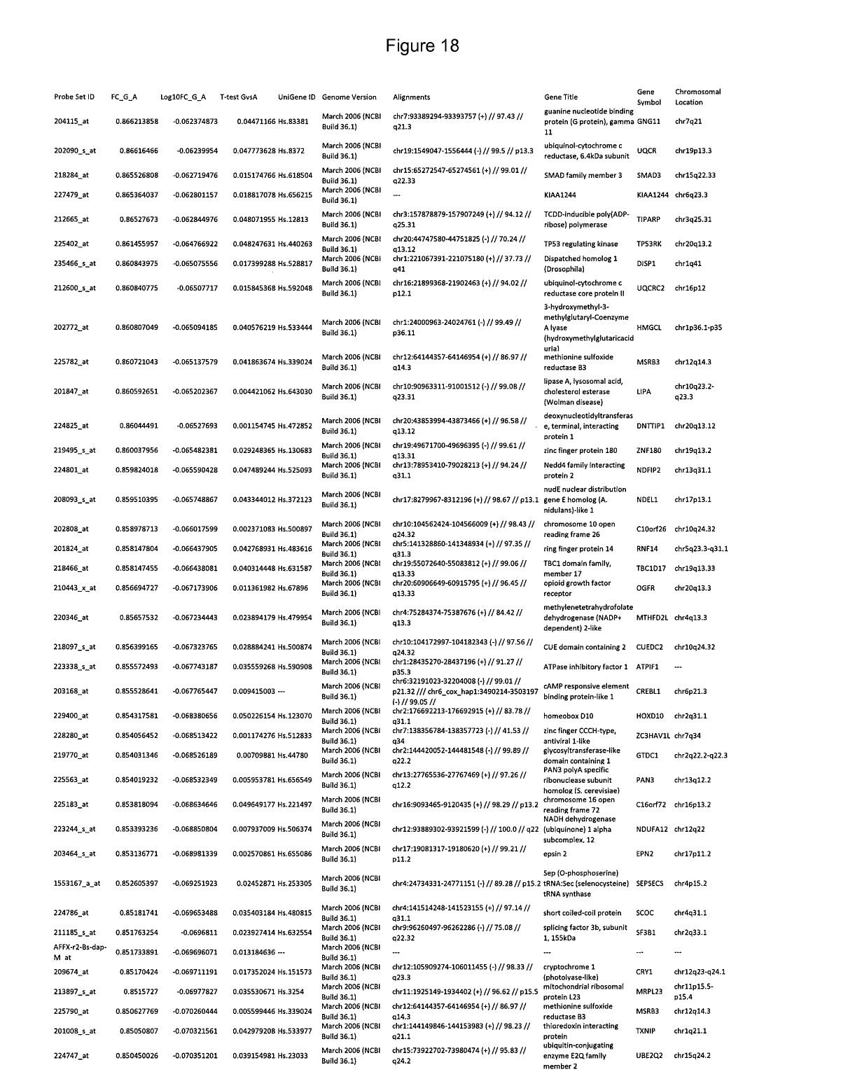

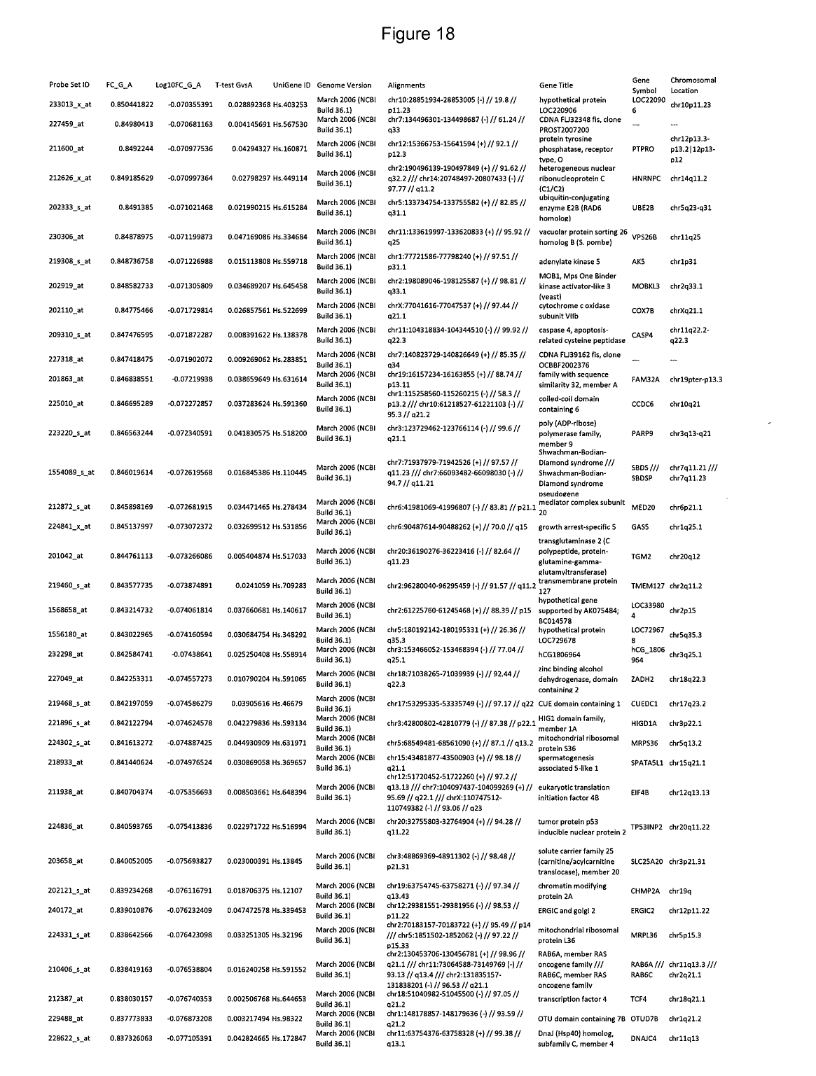

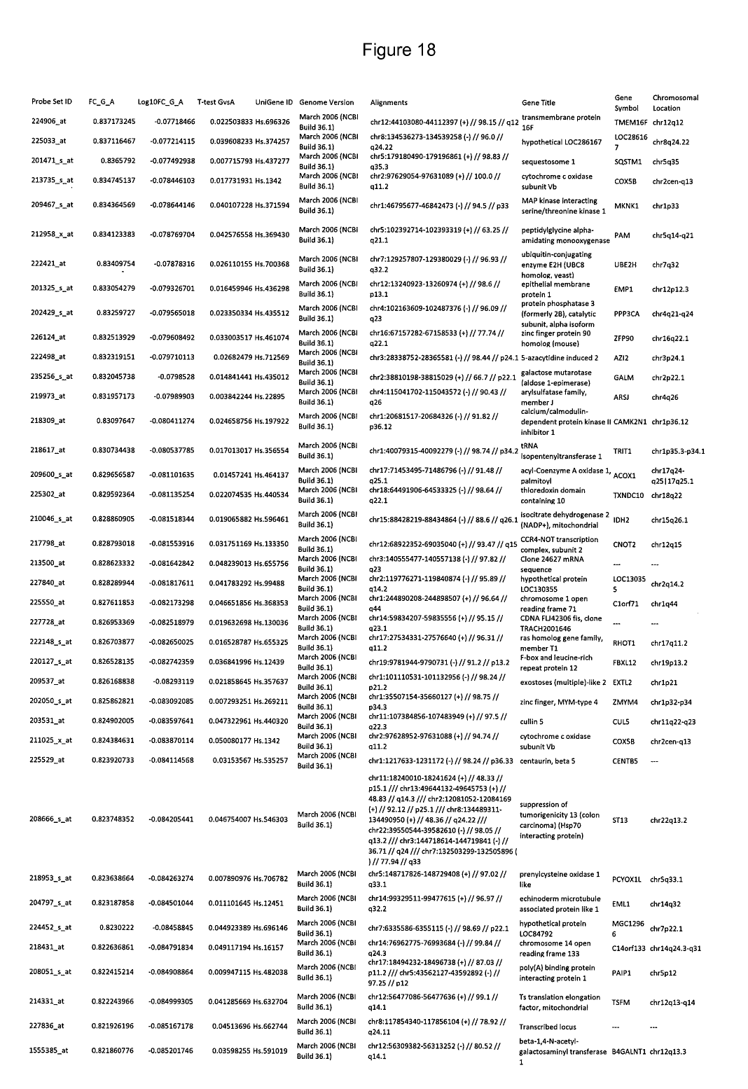

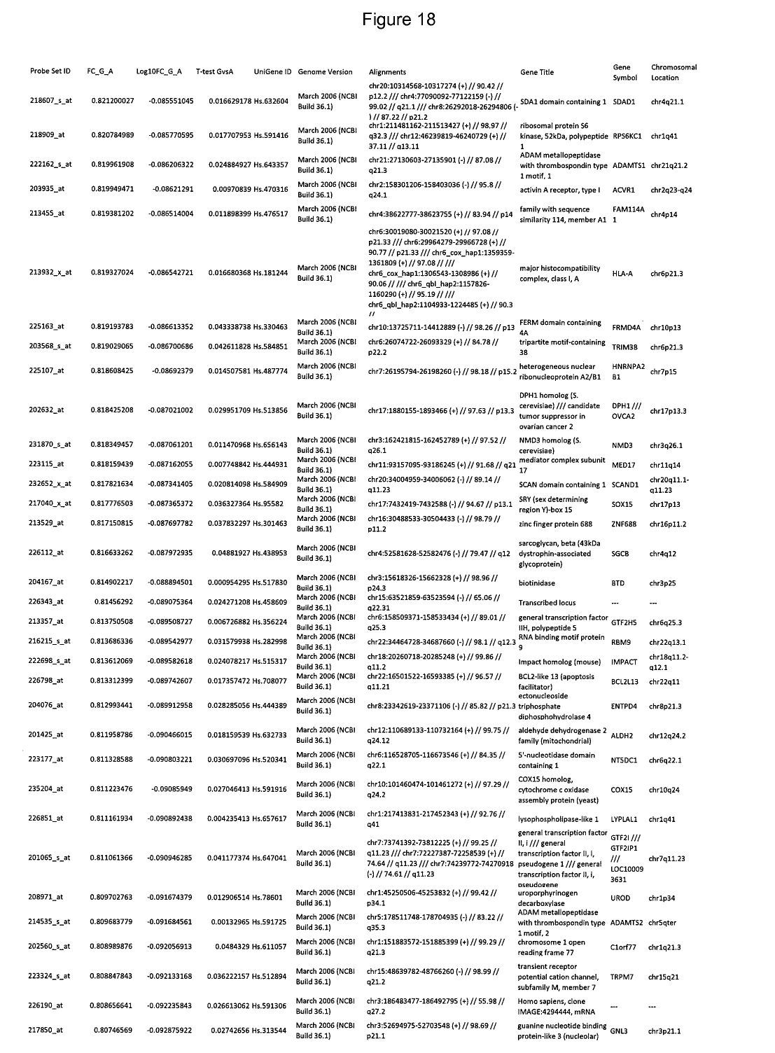

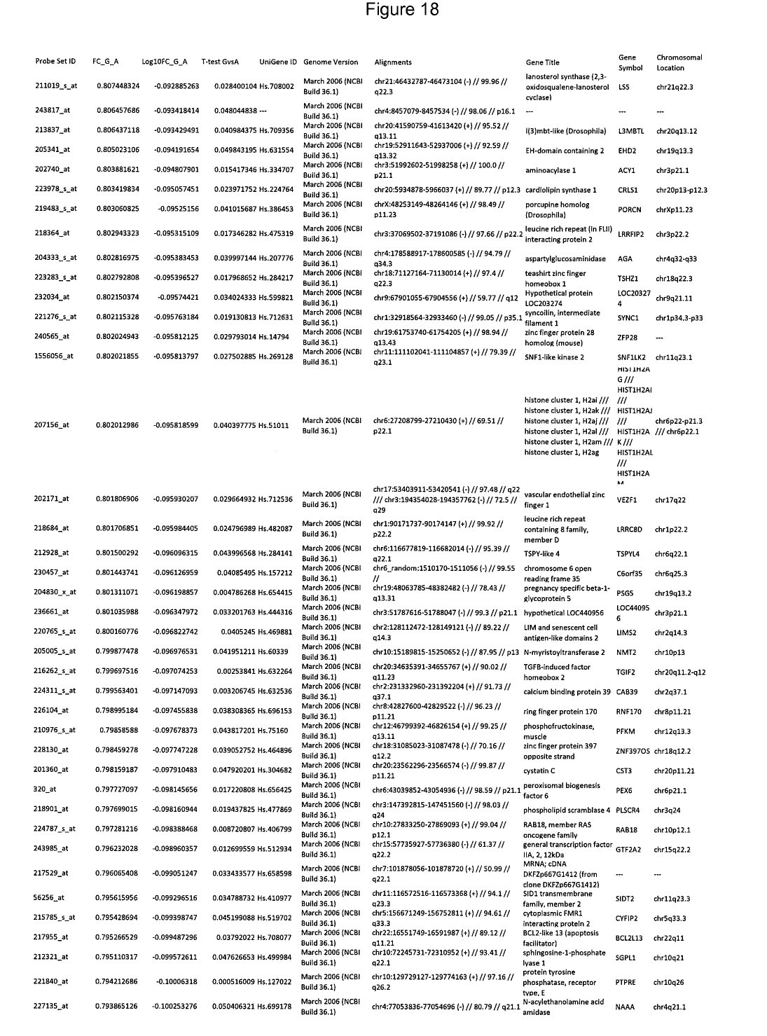

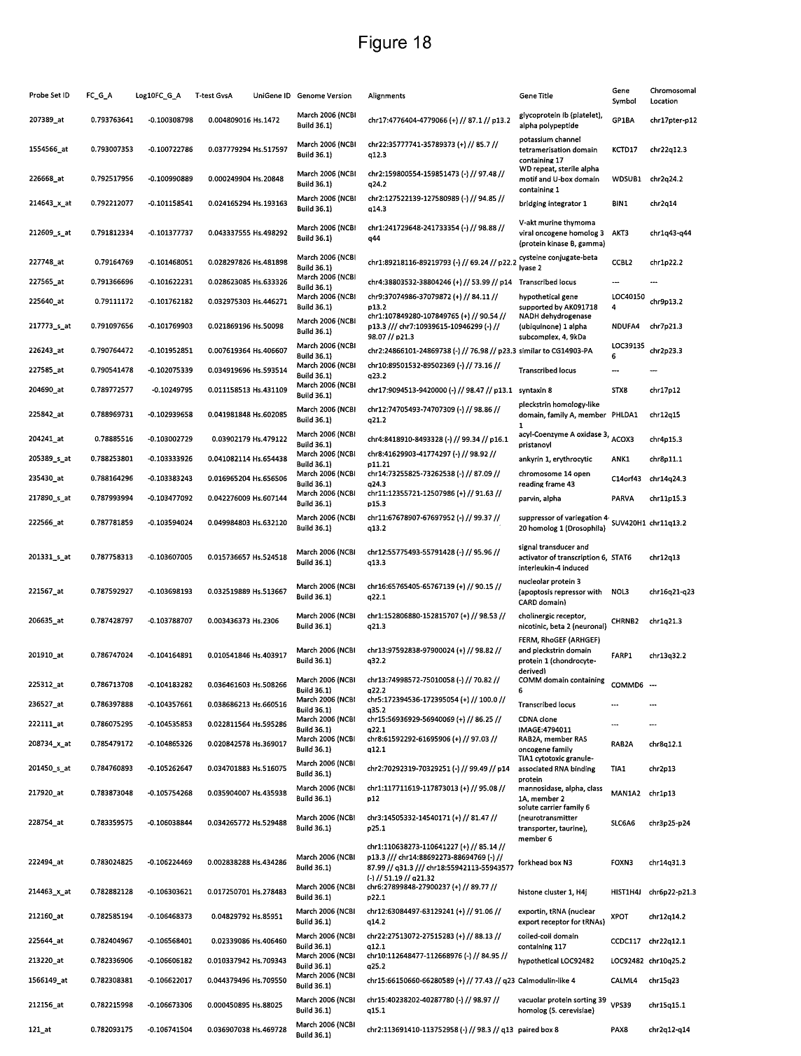

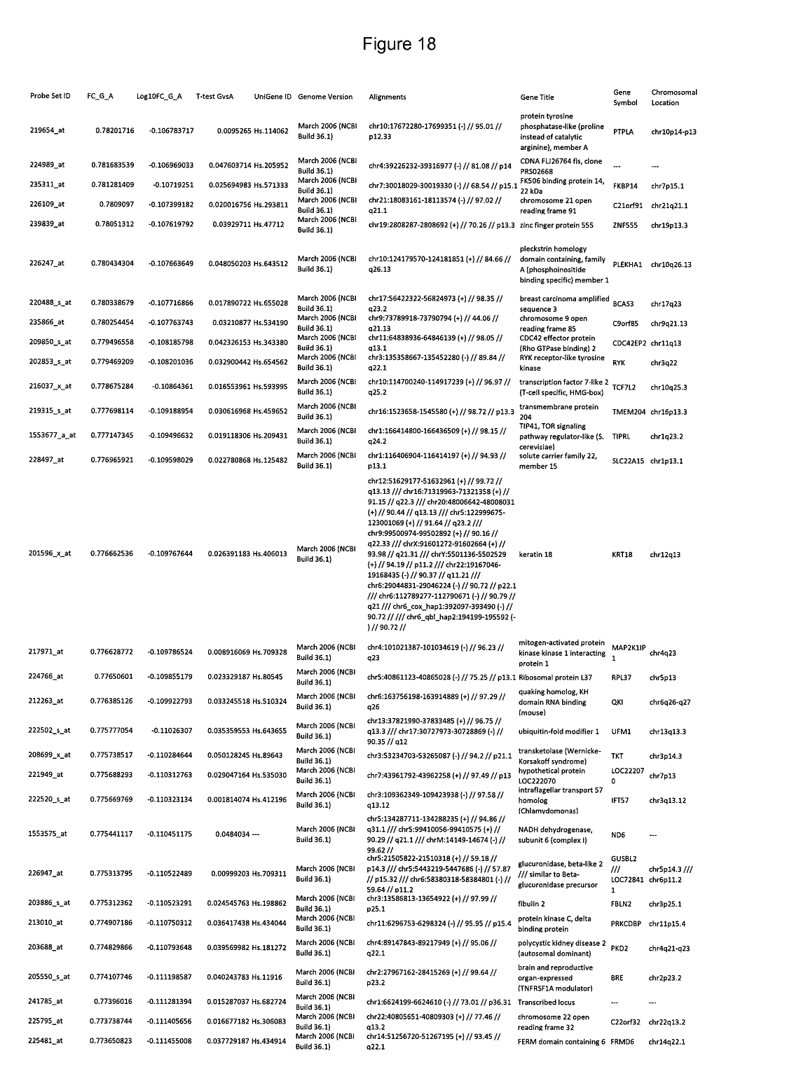

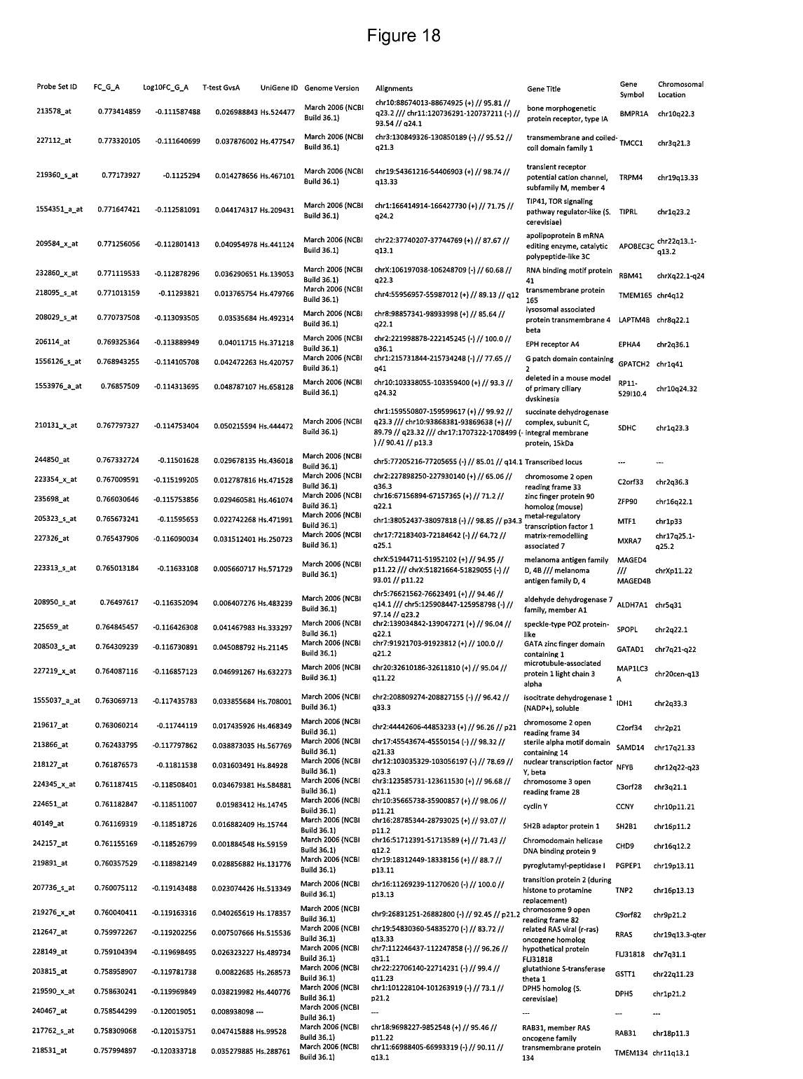

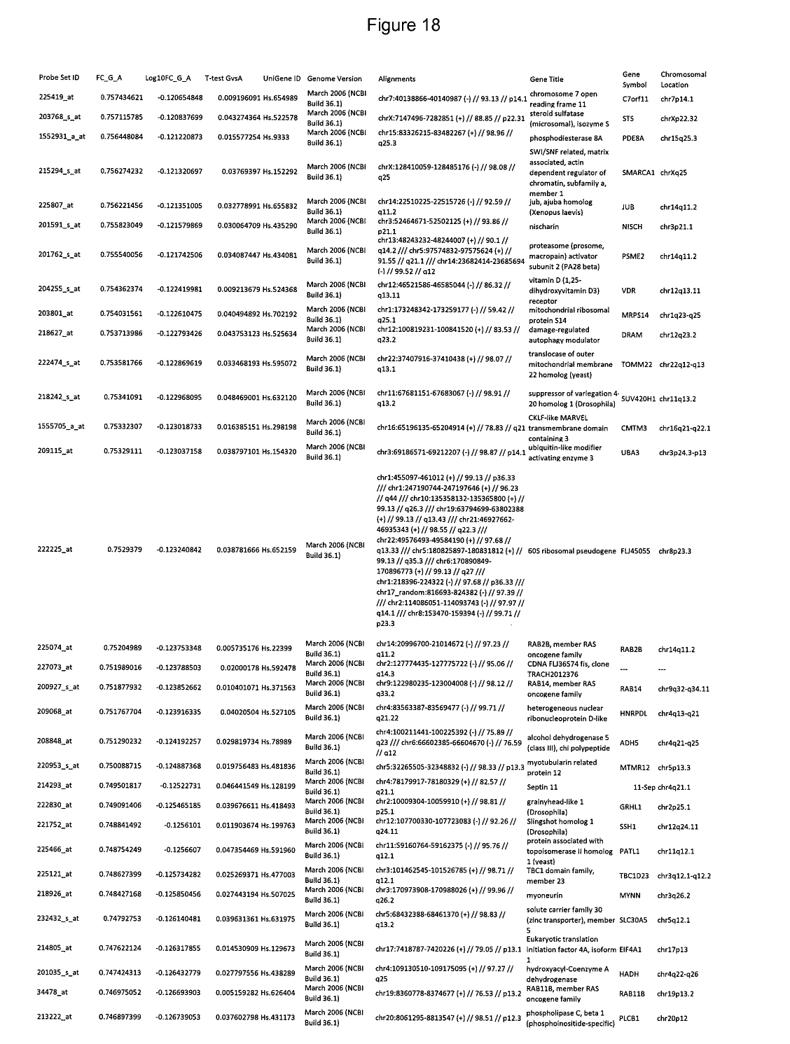

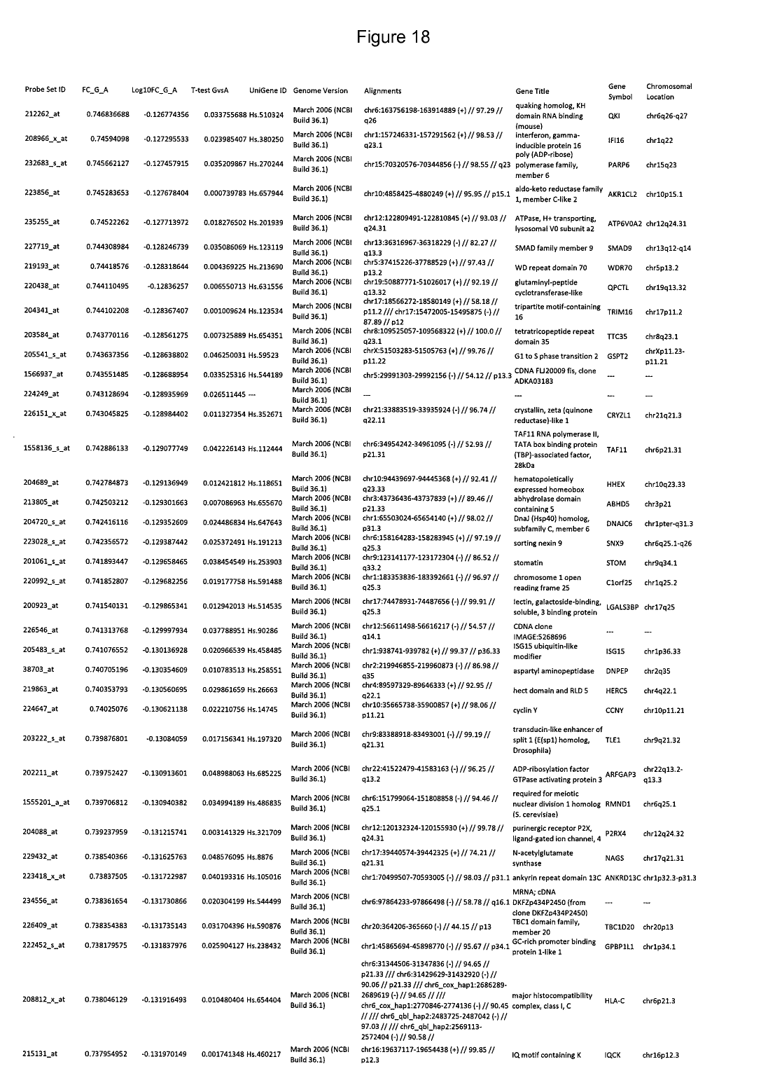

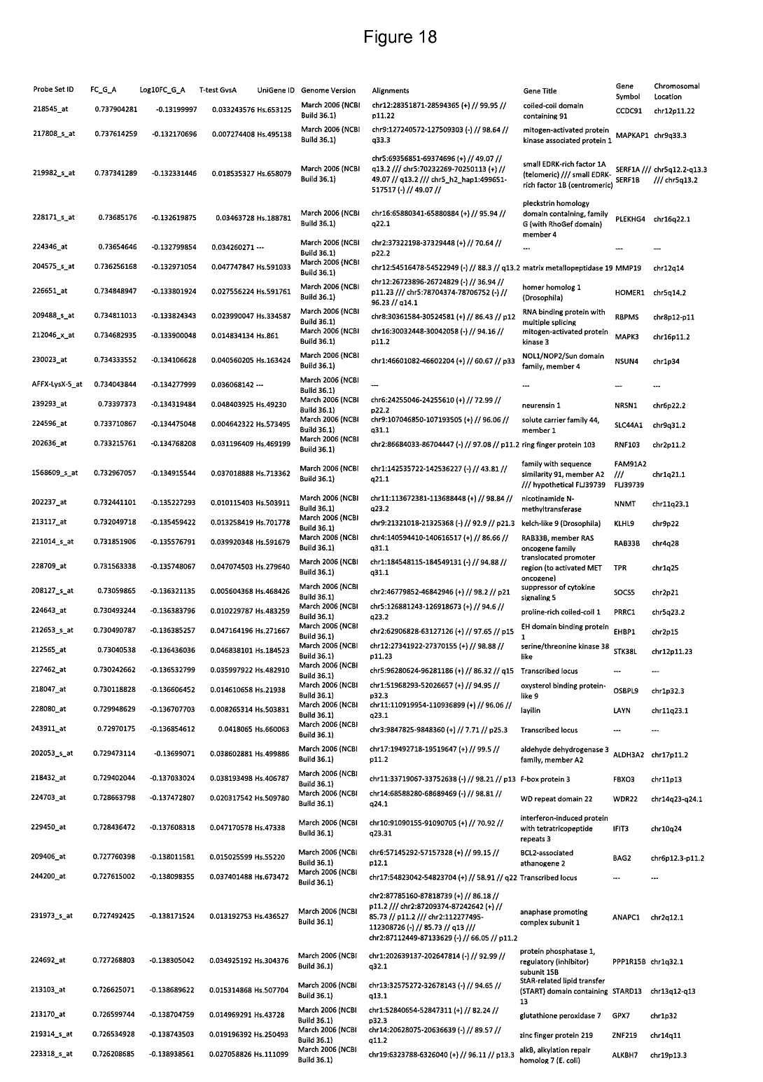

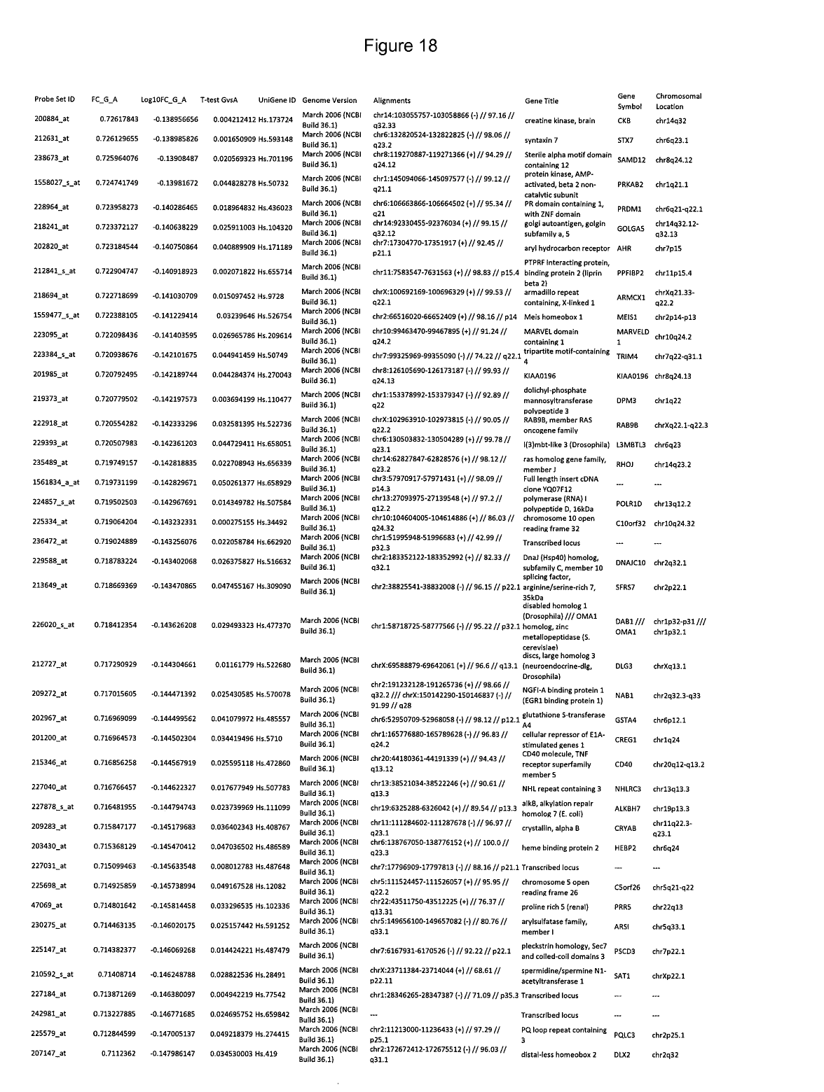

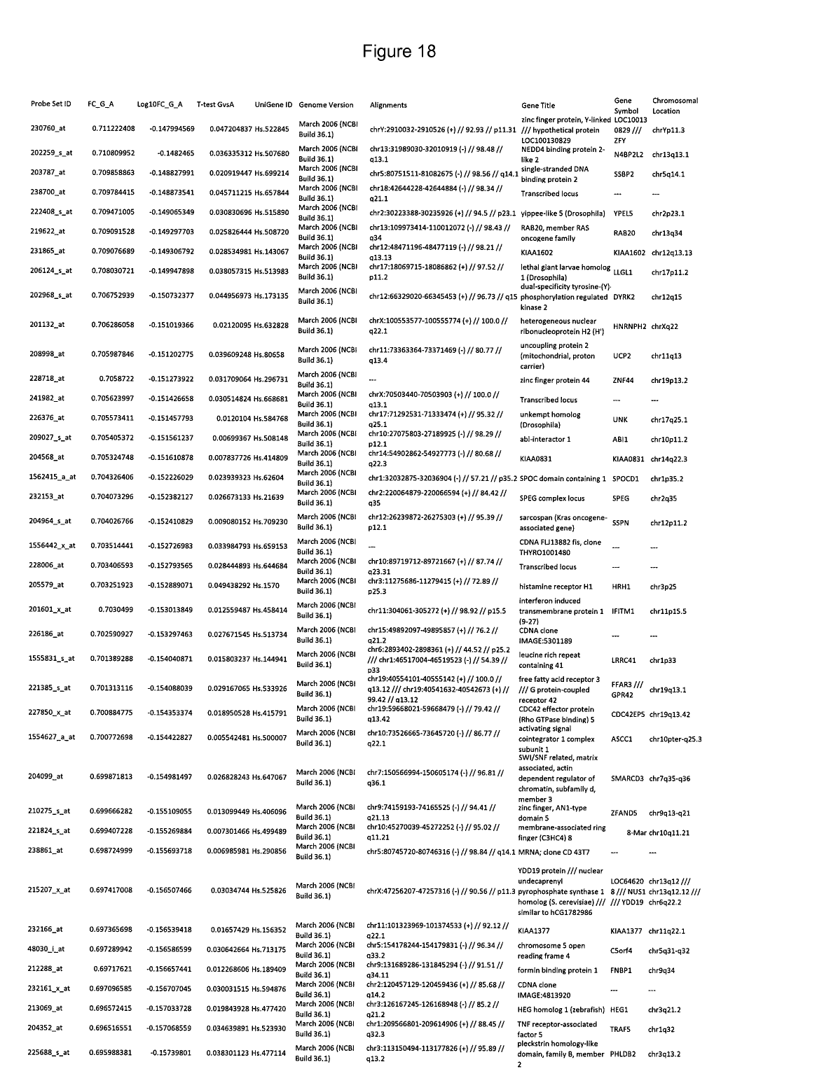

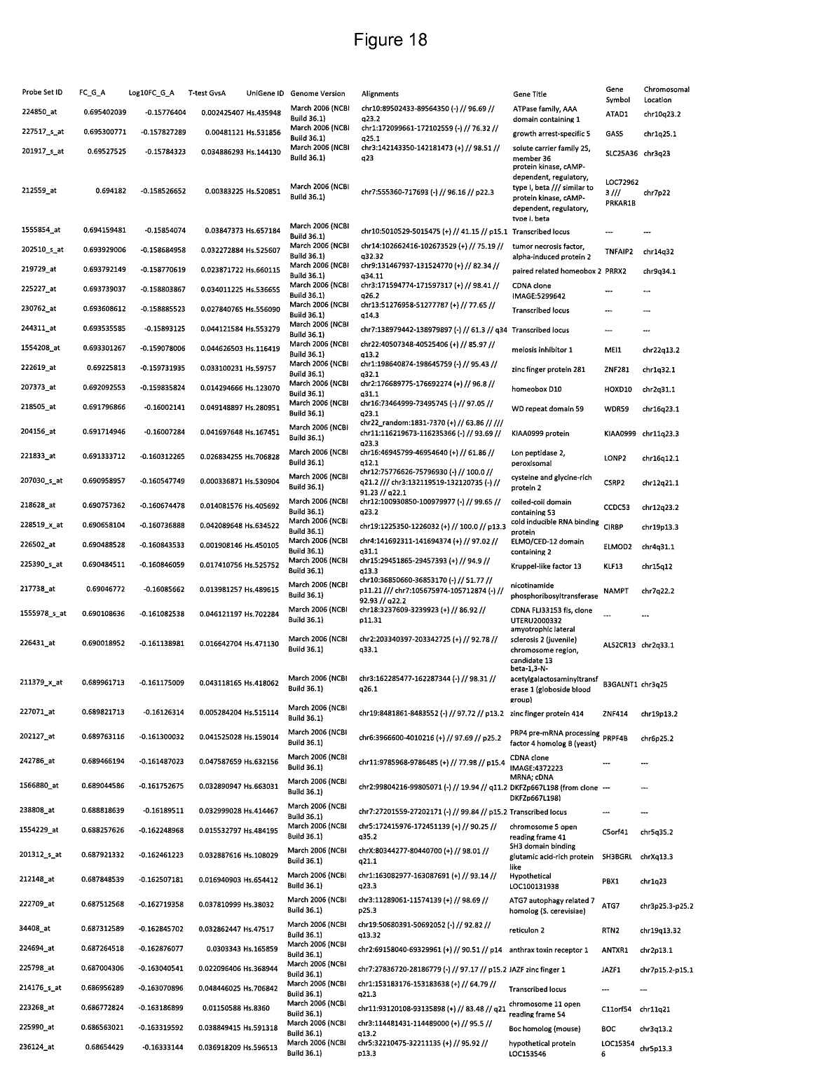

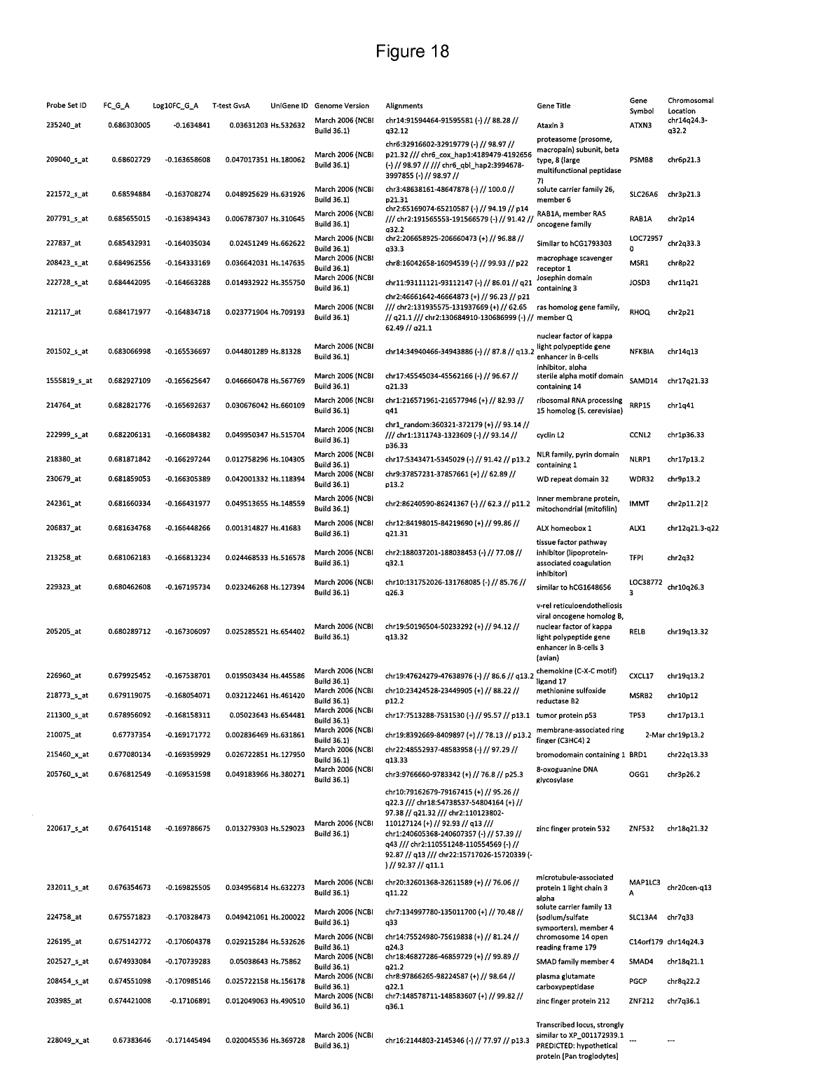

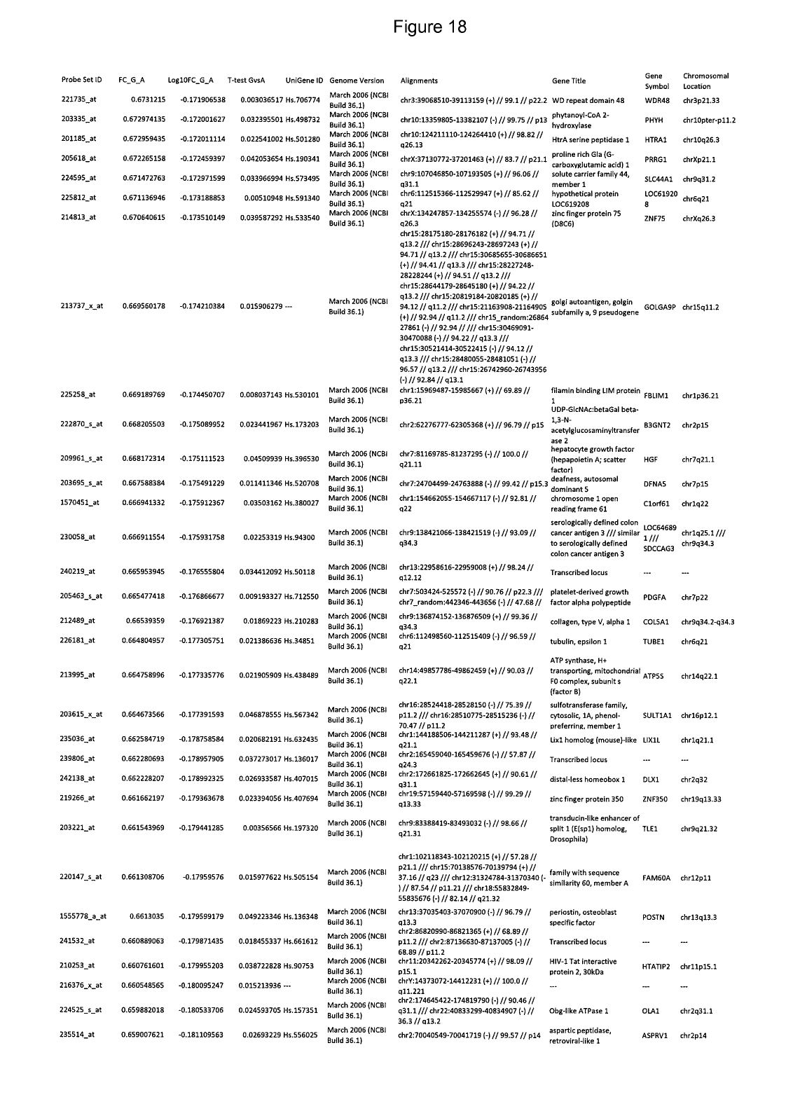

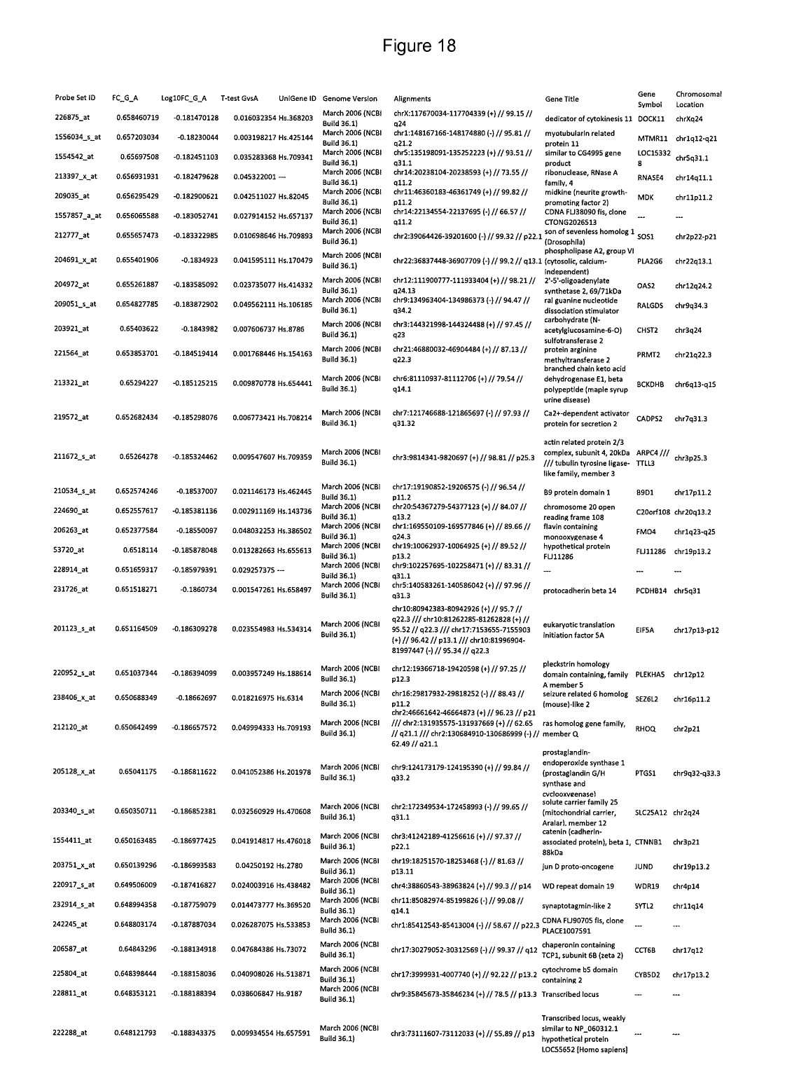

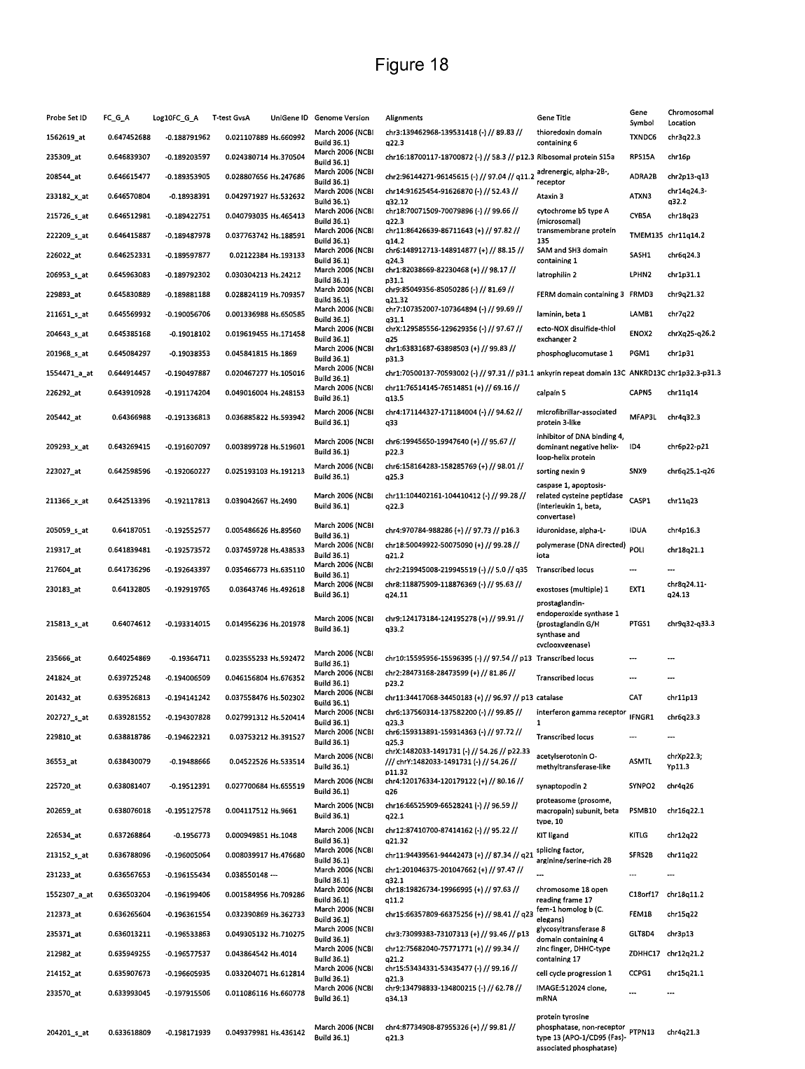

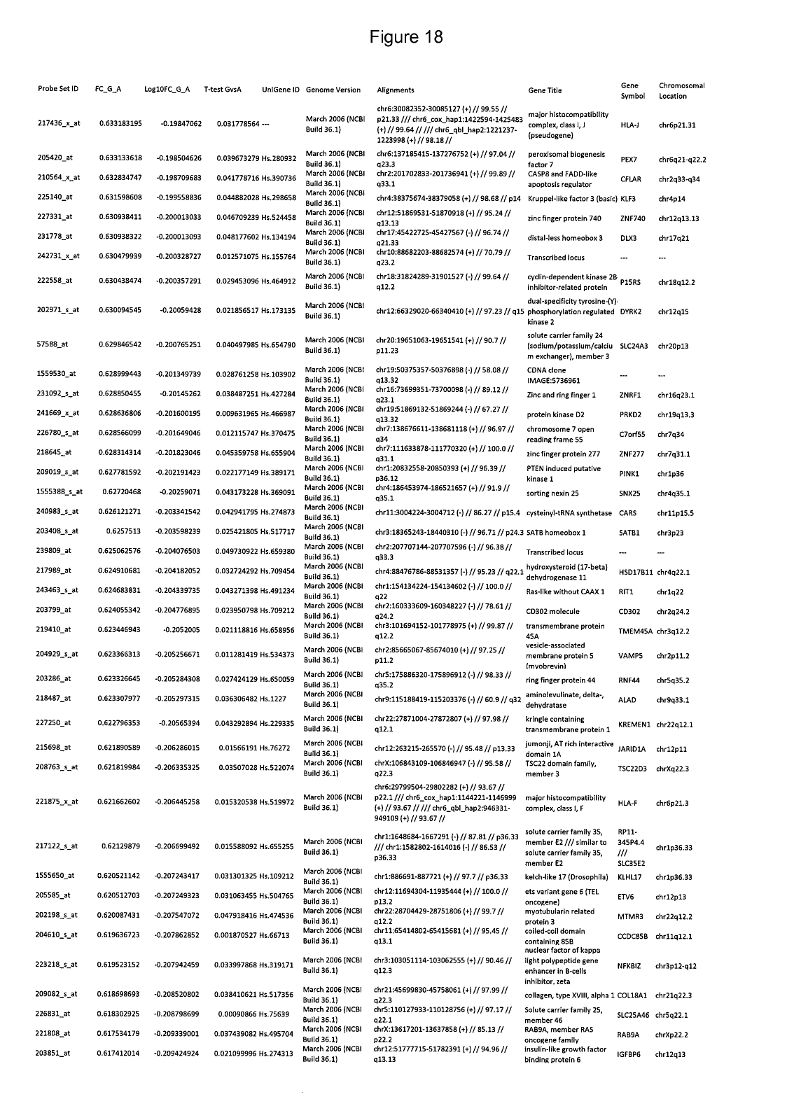

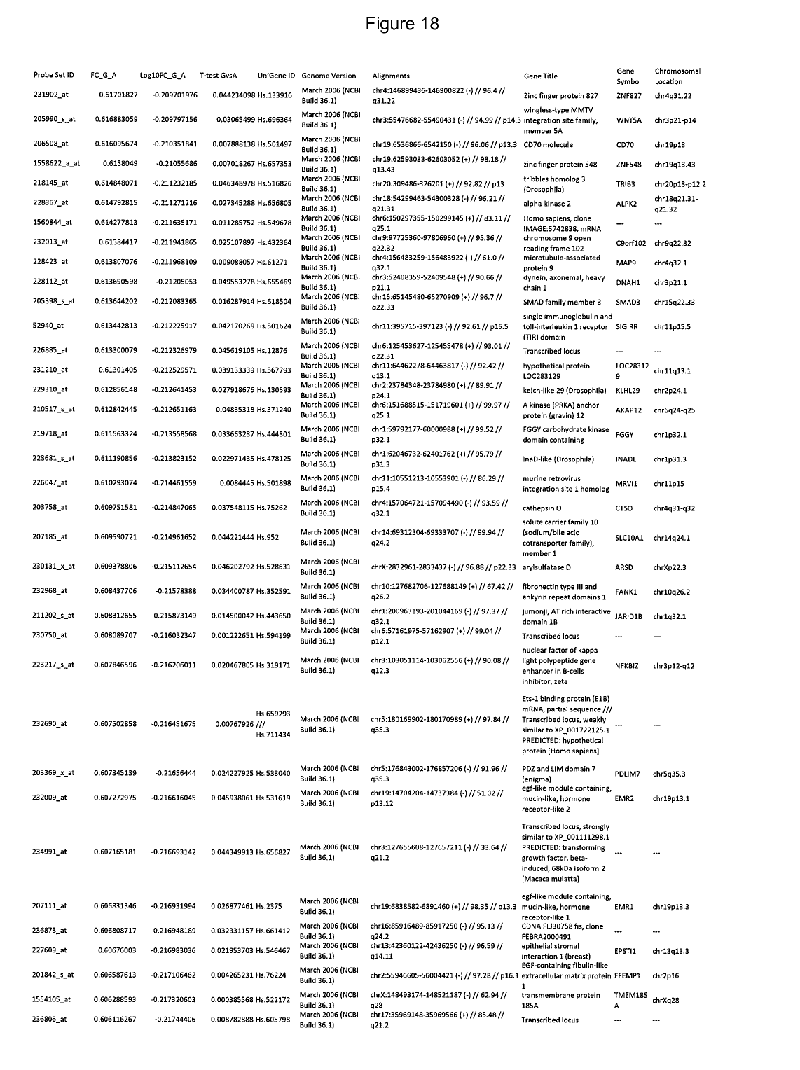

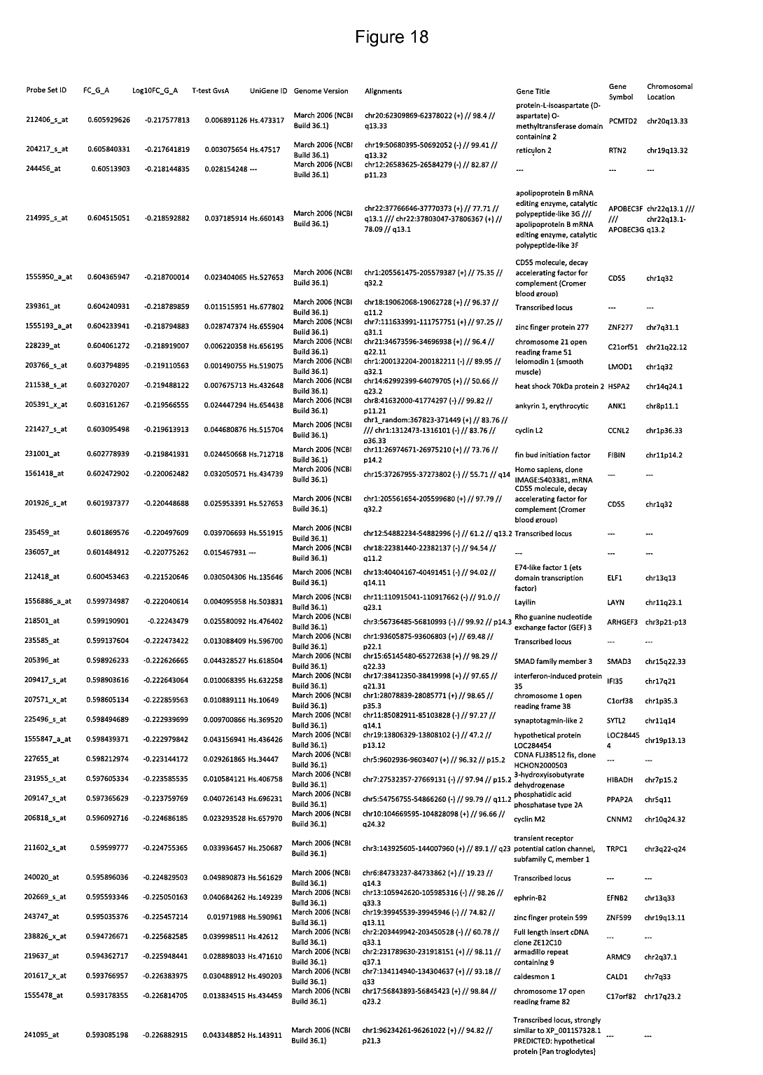

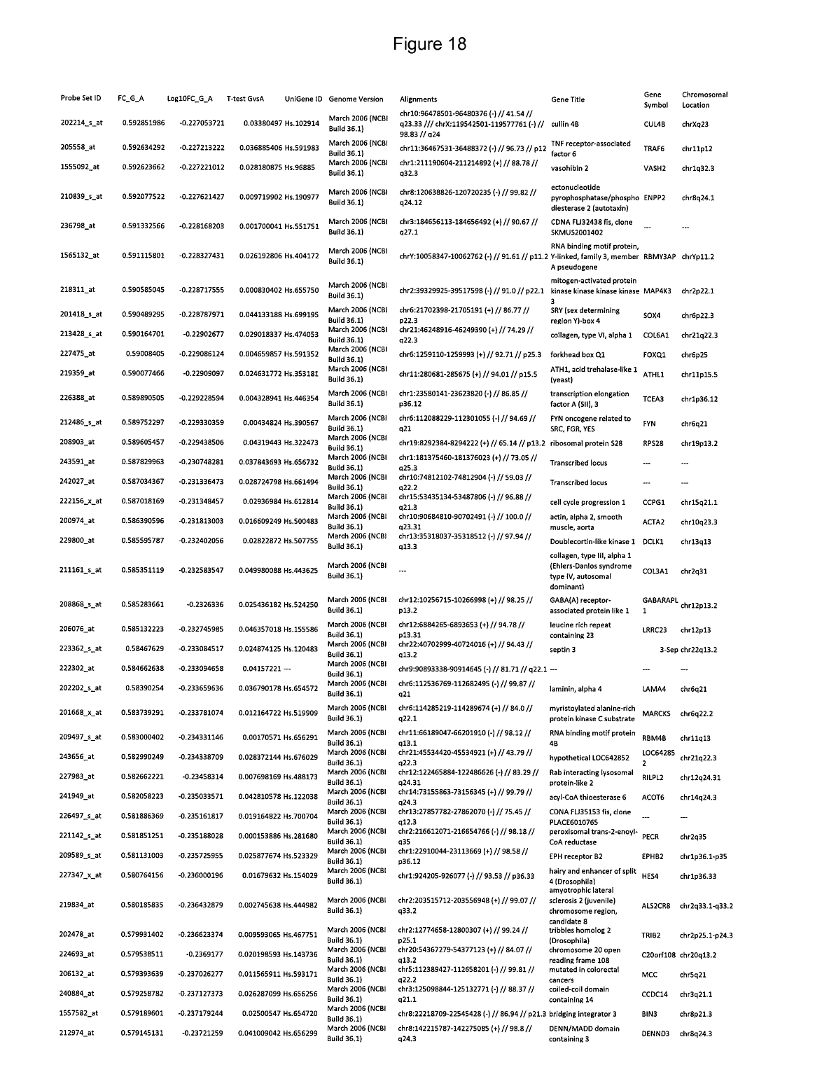

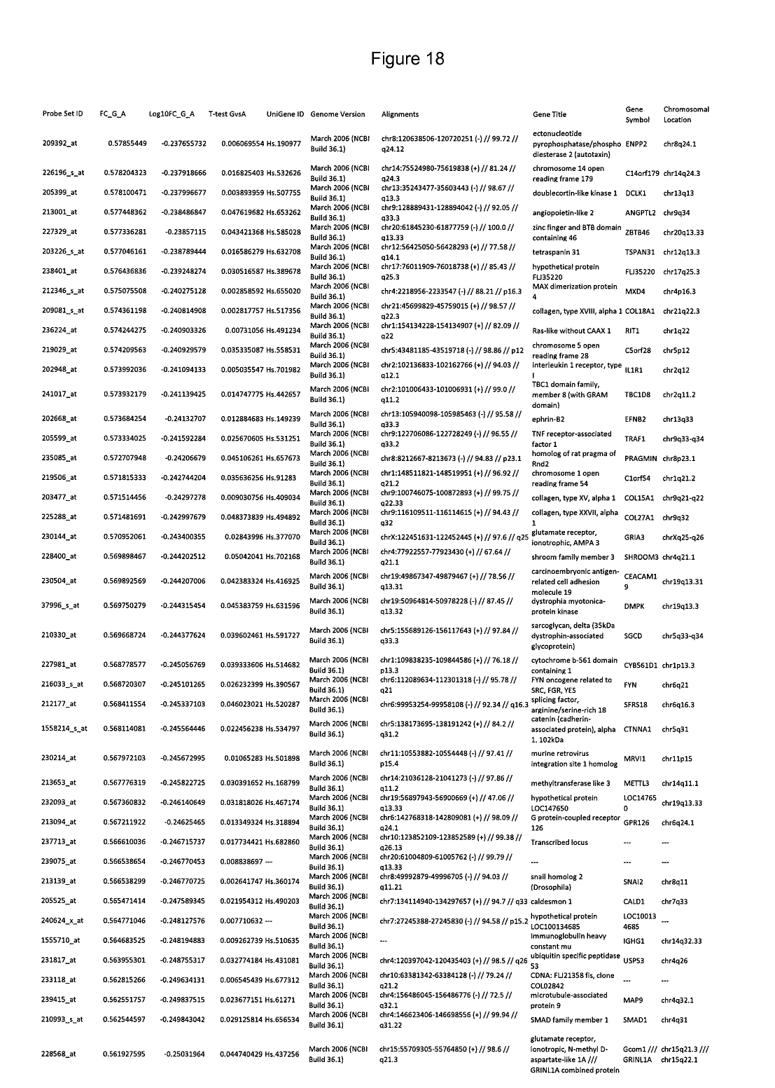

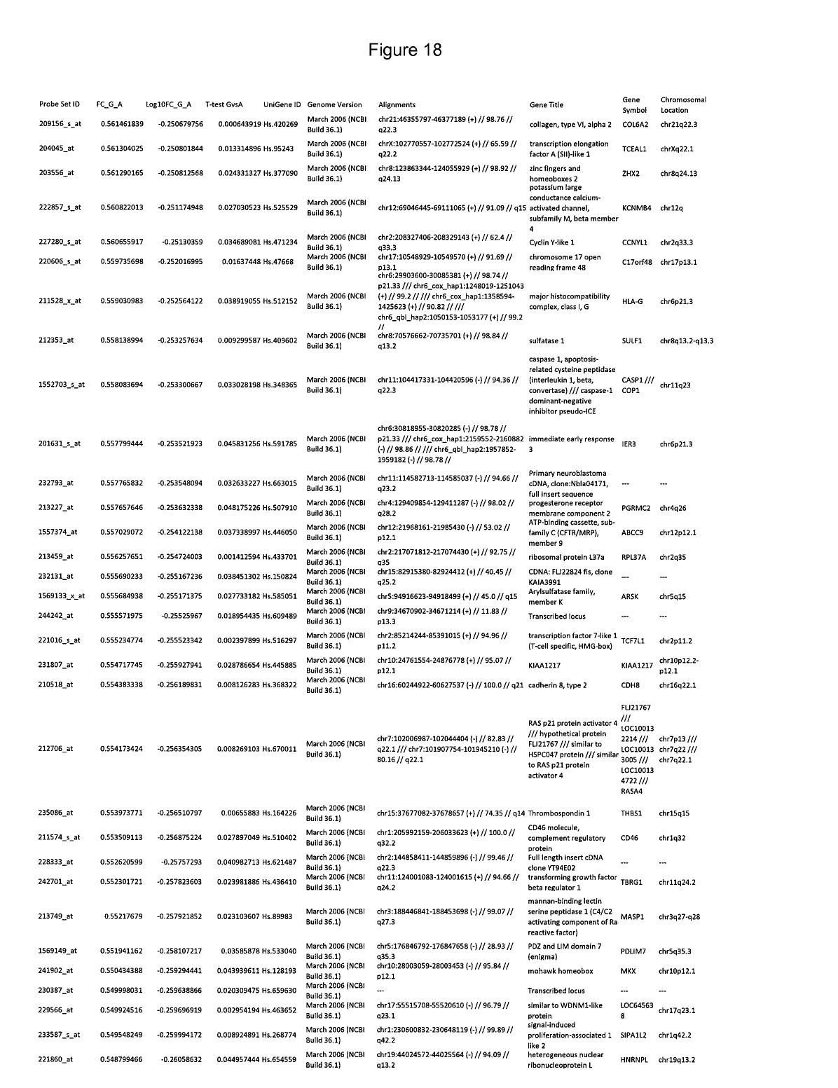

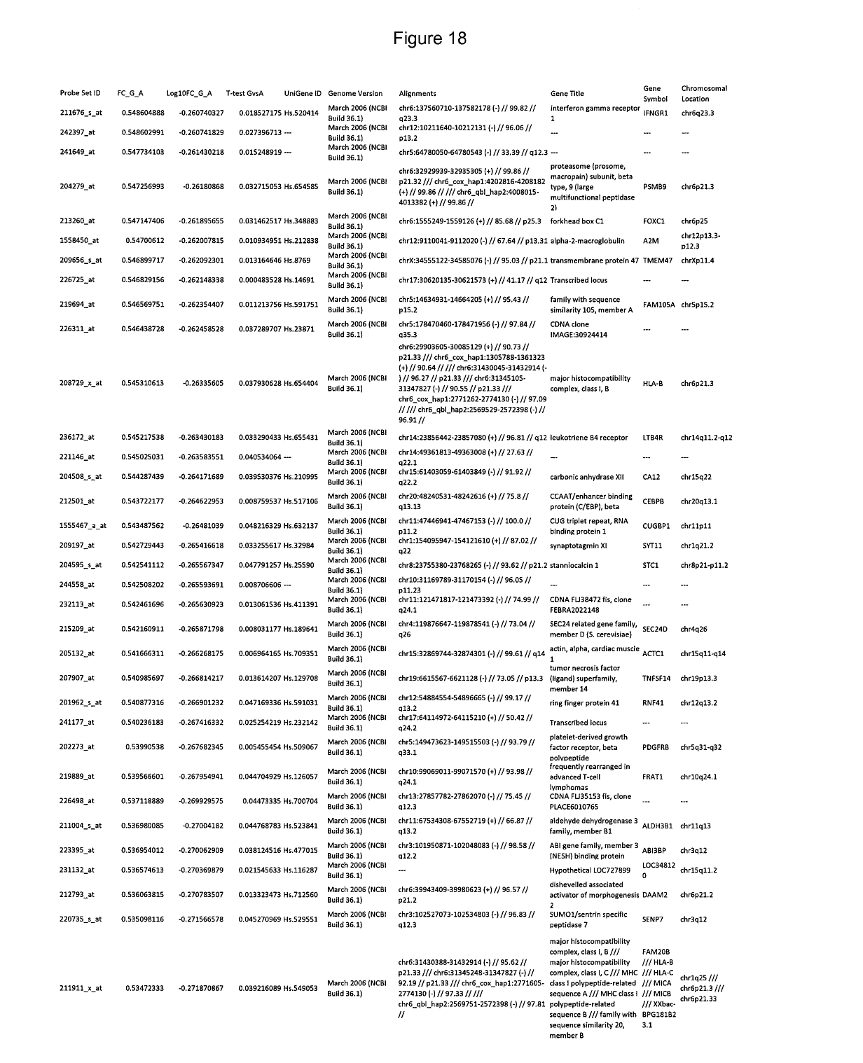

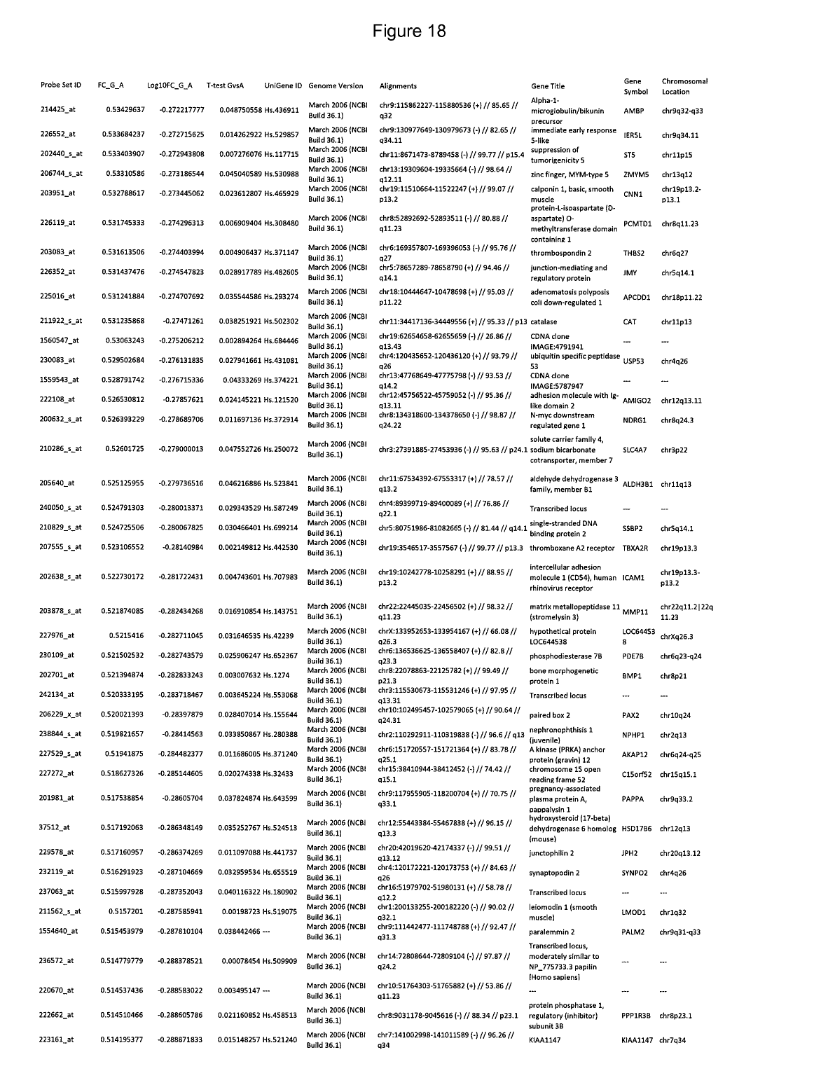

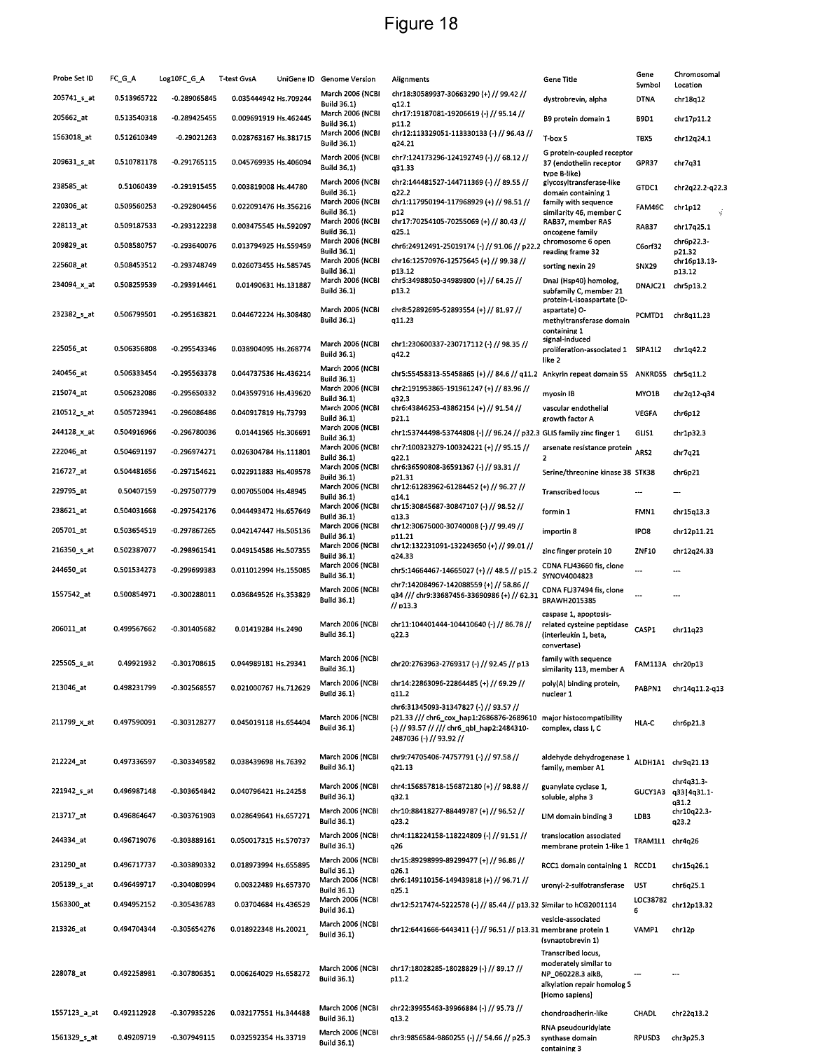

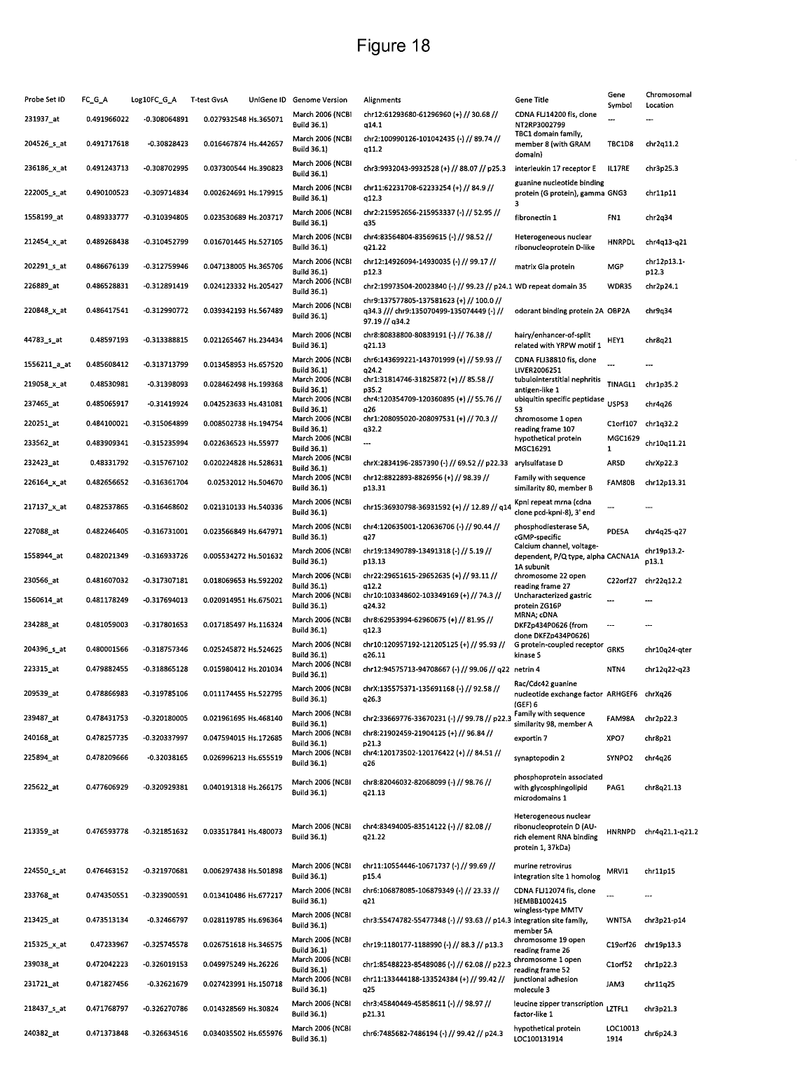

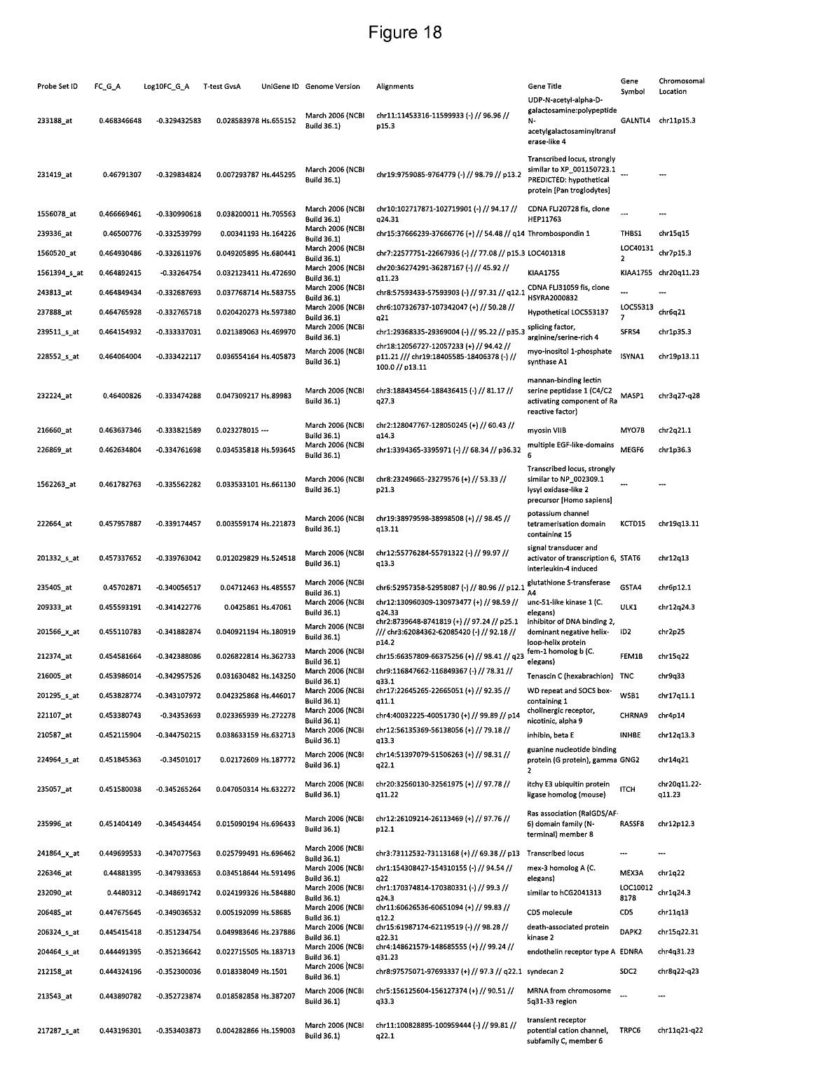

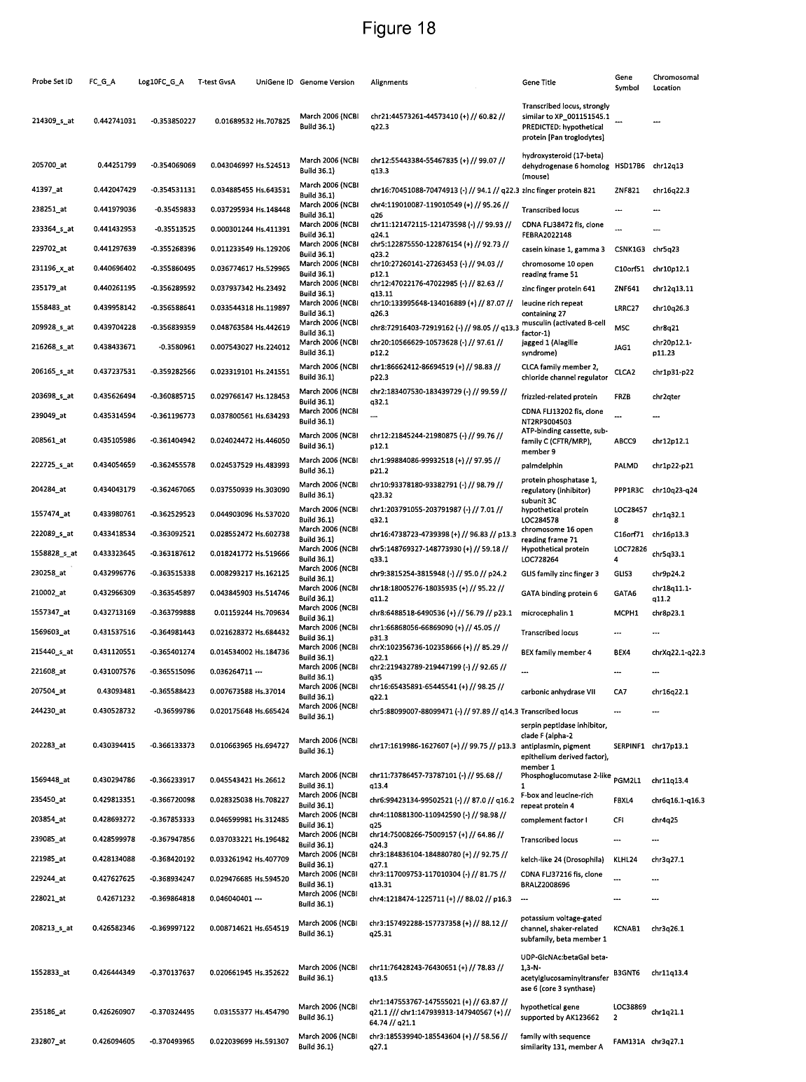

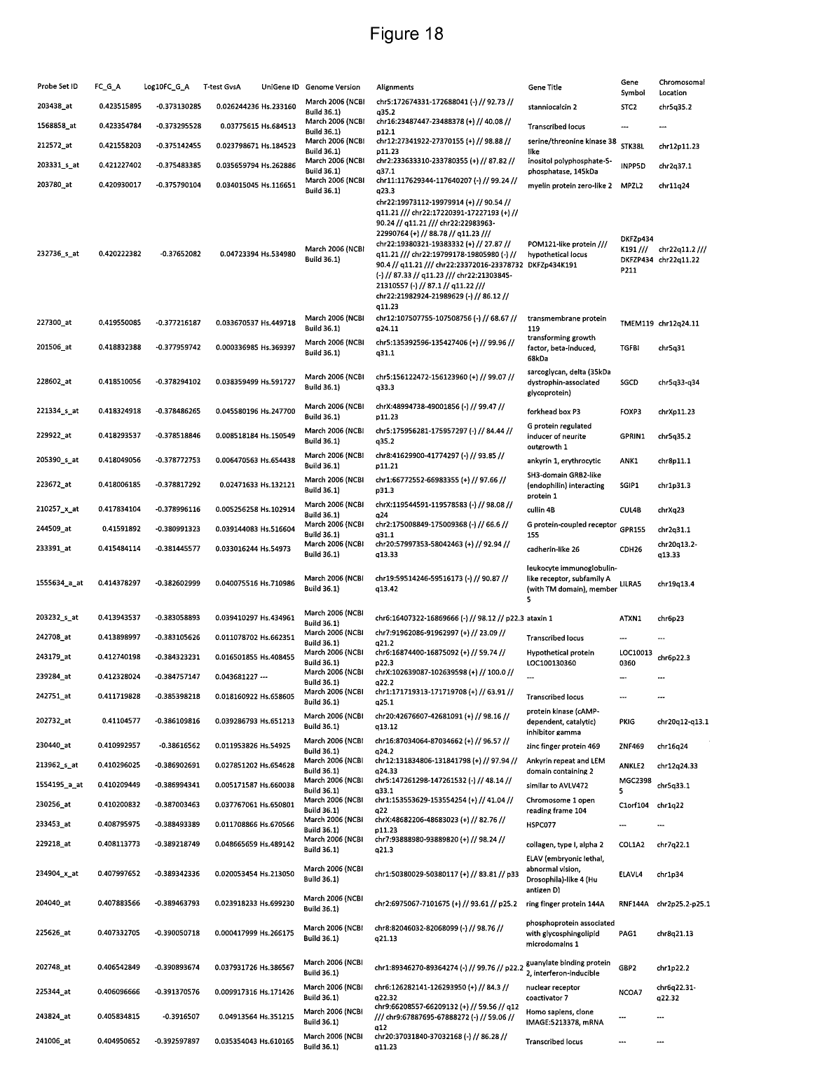

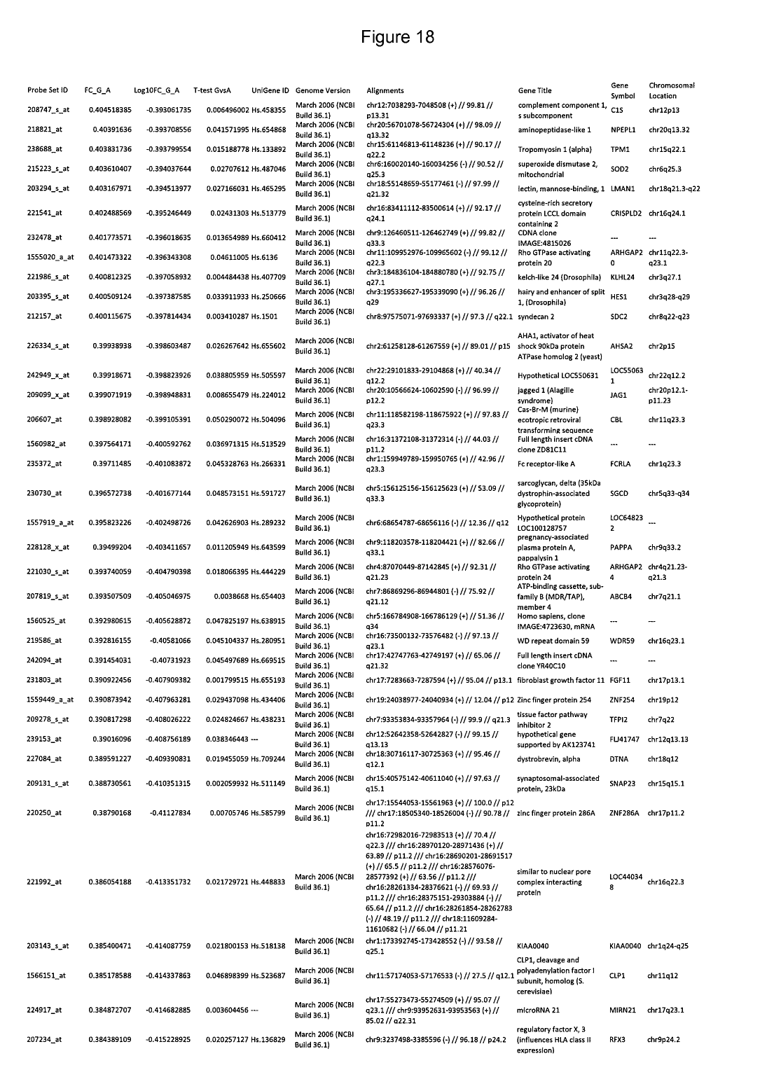

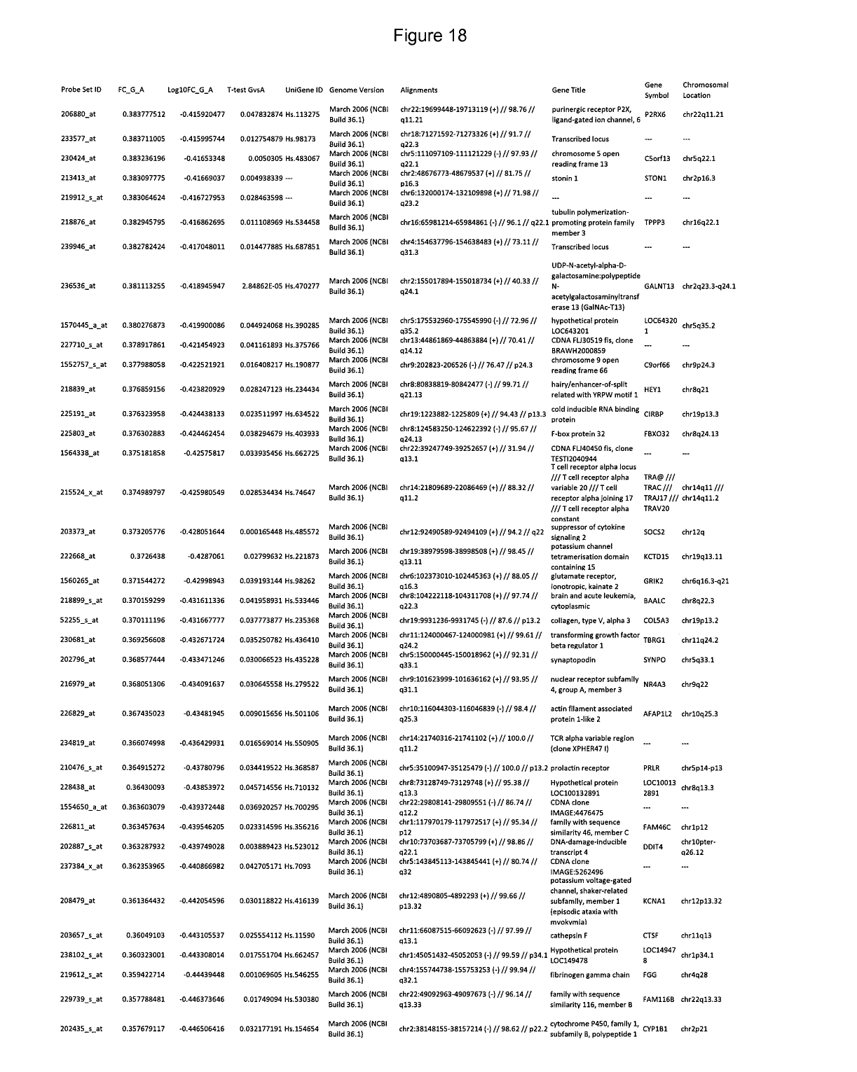

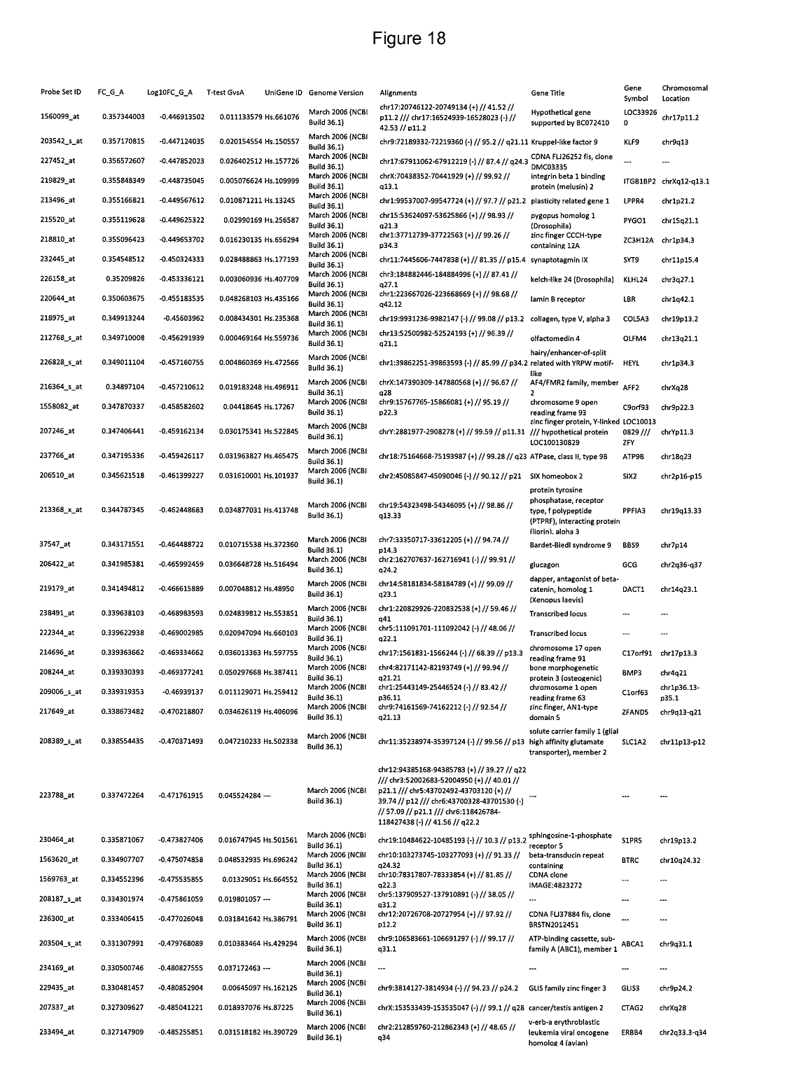

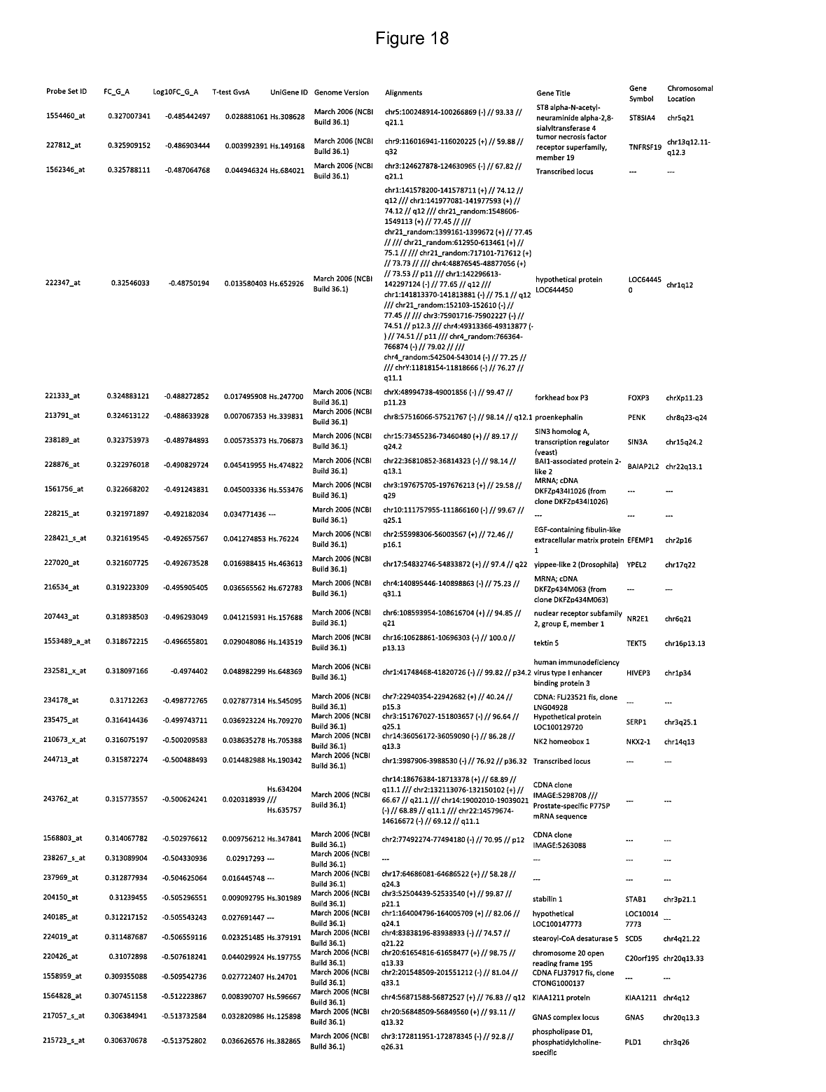

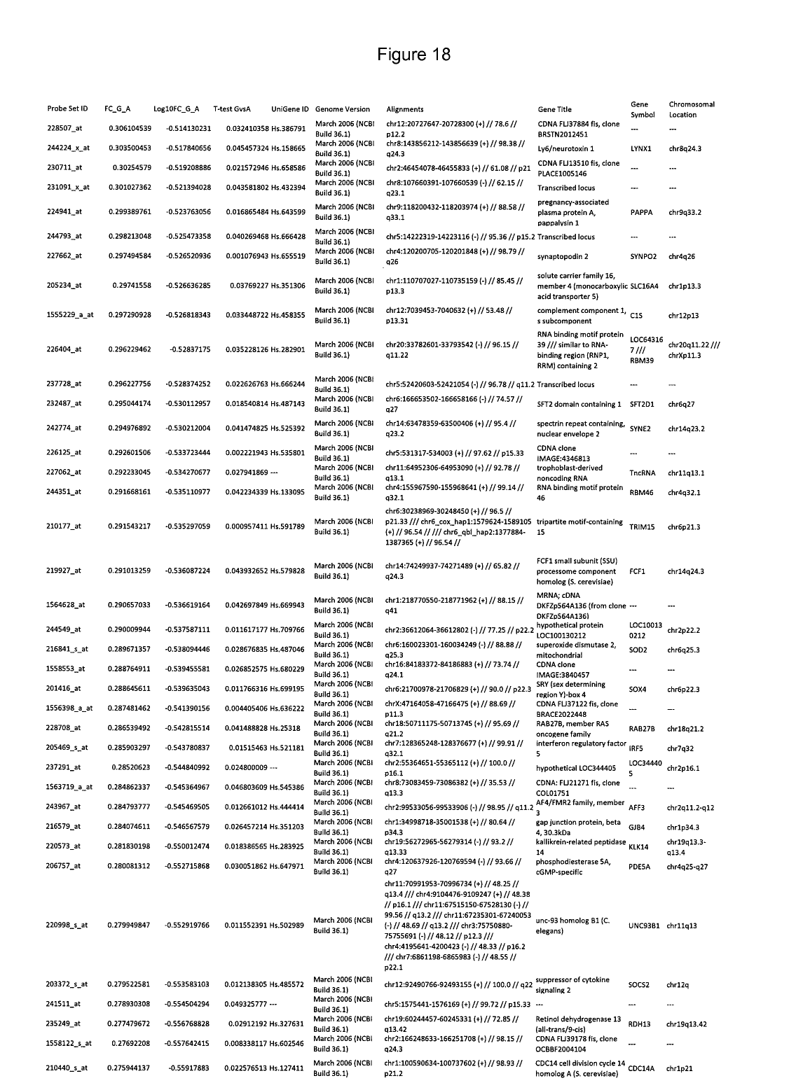

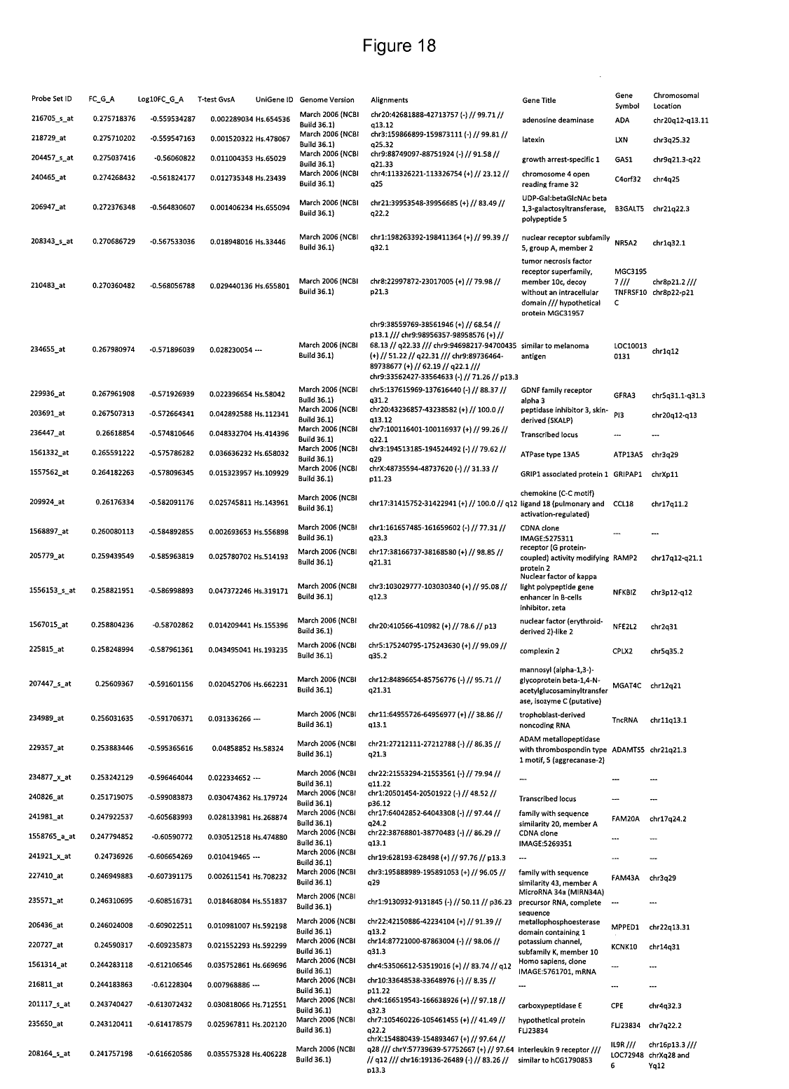

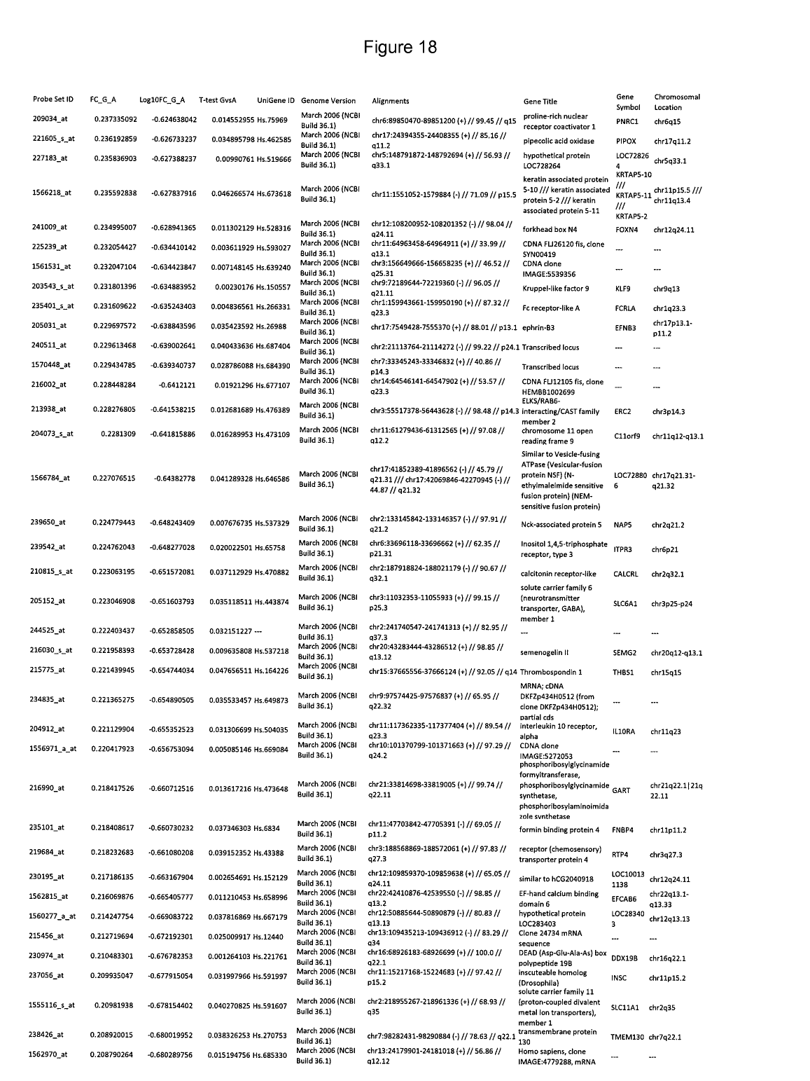

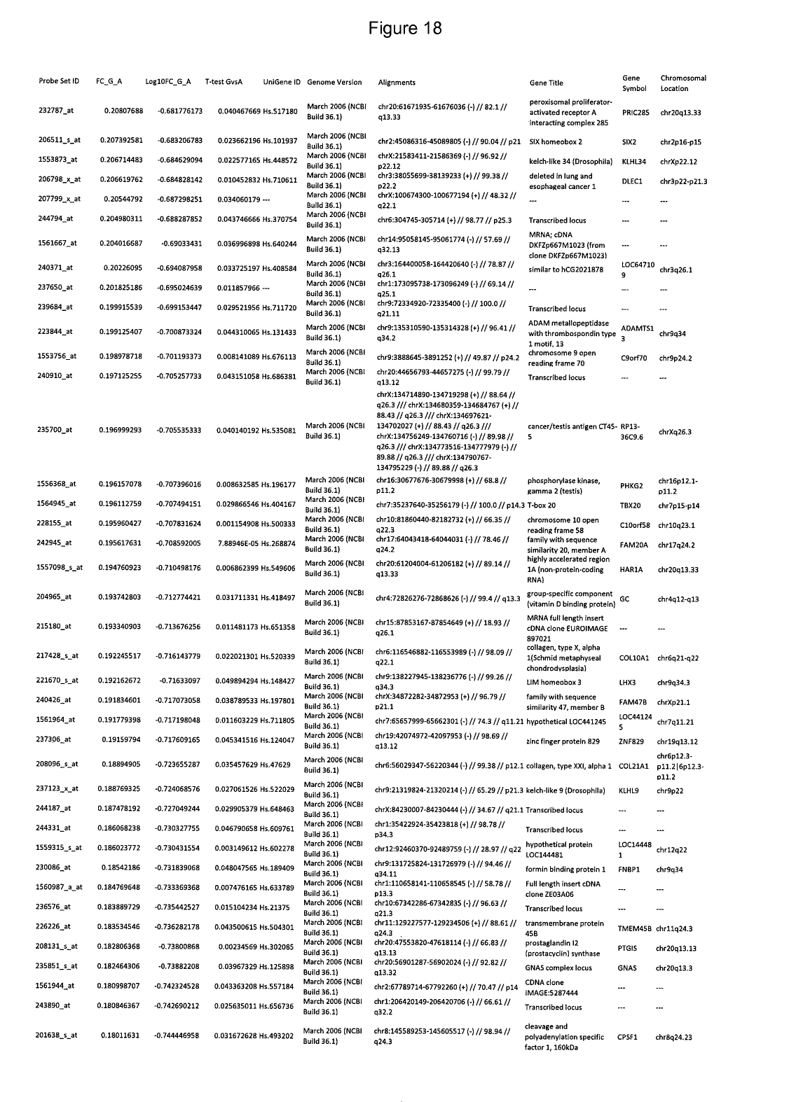

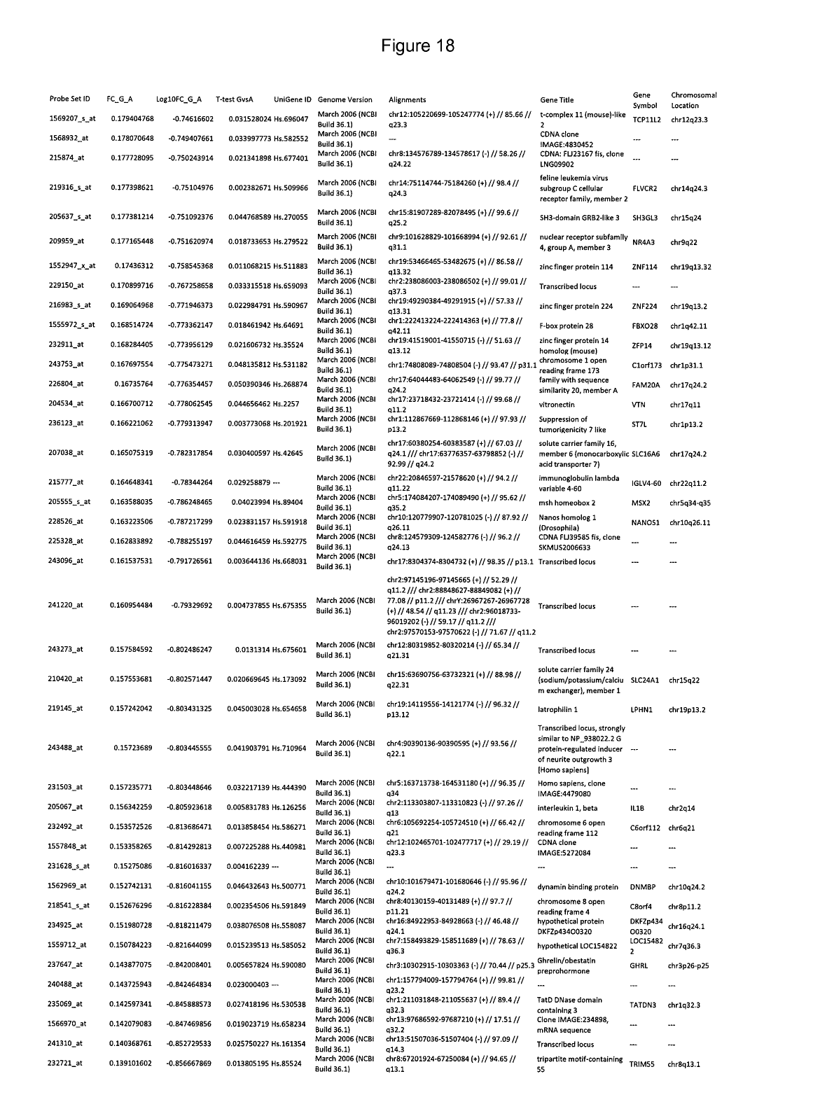

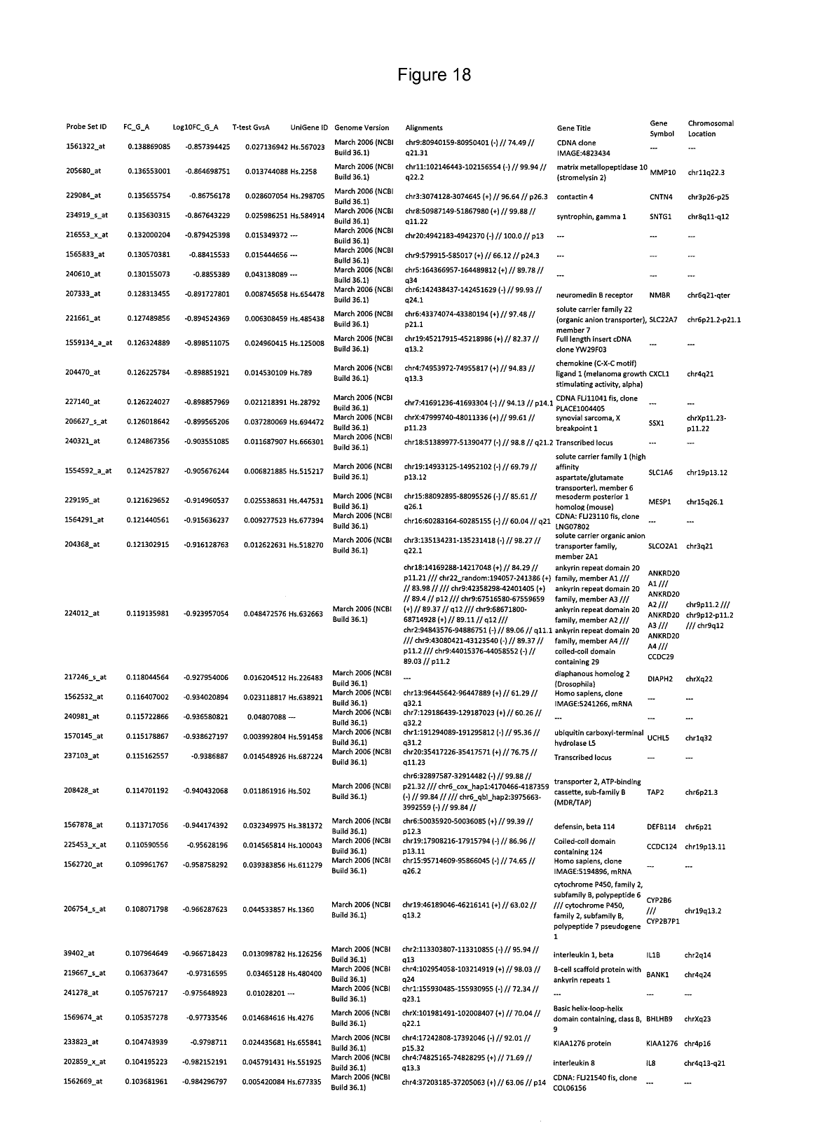

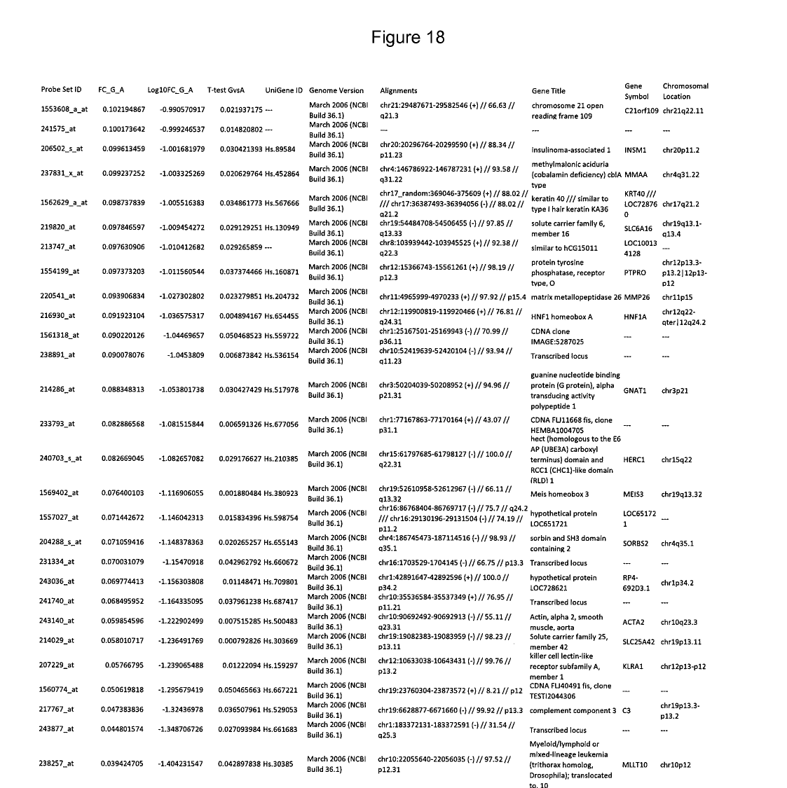

An RT-PCR-based screening protocol was used to identify RNA molecules encoded by disease-associated SNP sequences. This protocol was initially used to identify RNAs 100 to 200 nucleotides in length encoded by intergenic SNPs associated with multiple common human disorders including Crohn's disease, rheumatoid arthritis, type 1 diabetes, vitiligo, and multiple types of epithelial malignancies (prostate, breast, ovarian, and colorectal cancers). RNAs identified in the initial screen using human cells of mesenchymal (BJ1) and lymphoid (U937) origin are shown in FIGS. 1 and 2. The sequences of these RNA molecules are represented by their respective cDNA sequences in Table 1, supra. Tables 1 and 3). Further experiments also included human cells of epithelial origin (RWPE1) (FIG. 15, Tables 1 and 3). The results demonstrate the cell-type specific expression of many of the small RNAs.

The RT-PCR based screening protocol comprised the following steps: extraction of small RNA from cells; determination of DNA contamination by PCR for beta-actin; synthesis of cDNA; first PCR using primer set 2 (GC2F and GC2R); nested PCR of purified first PCR product using primer set 1 (GC1F, GC1R); gel purification of final PCR product; confirm sequence of final PCR product by direct sequencing. Detailed protocols are found infra, in the section entitled Materials and Methods.

Further analysis identified a subset of sequences flanked by the same protein-coding genes in both human and mouse genomes. These sequences are selected from A6, A9-11, A16, A23, B6, C12, D2, D5, D26, E3, E12, and the rs2670660 (NALP1 Loci) RNAs, all of which are shown in Table 1, supra. Further analysis using genome-wide chromatin domain maps (see Kim et al., Nature 465:182-87 (2010) and Ku et al., PLoS Genet. 4:e1000242 (2008) suggested that these intergenic disease-associated genetic loci represent Polycomb-regulated intergenic chromatin domains.

Analysis of the predicted secondary structures of these RNA molecules revealed the presence of loop sequences containing SNP-bearing segments of 8-11 nucleotides in length which are identical to primary sequences of microRNAs (FIG. 2B). The loop structures of the allelic variants also are predicted to have distinct secondary structures. The RNA molecules contain multiple potential target sites for microRNAs which are often clustered around SNP nucleotides. These data suggested an epigenetic regulatory cross-talk between the intergenic RNAs and microRNAs. As shown infra, microarray expression profiling of human cell lines stably expressing distinct allelic variants of the NALP1-locus SNP rs2670660 RNAs identified microRNAs whose expression was differentially regulated by the '660 RNAs in an allele-specific manner.

1.3 NALP1 Loci-Associated Intergenic SNP, rs2670660 Encodes Small RNAs that Cause Allele-Specific Changes in Human Cells

The NLRP1/NALP1 loci, including the hypothetical extended NLRP1 (NALP1) regulatory region, is strongly associated with vitiligo and multiple autoimmune and autoinflammatory disorders. One of the NALP1-associated SNPs, rs2670660, is of particular interest because it occurs within a segment of the genome that is remarkably conserved among species, including human, chimpanzee, macaque, bush baby, cow, mouse, and rat. Four sets of primers were designed to detect the predicted RNA molecules encoded by the rs2670660 sequences. The primer sequences (5′ to 3′) are as follows:

| Set 1: | |
|---|---|
| (SEQ ID NO: 326) | |
| (forward) | CACGCACAAGTGATCTACCAG |
| (SEQ ID NO: 327) | |
| (reverse) | GCATCAGGATVCACCAGTC |
| Set 2: | |
| (SEQ ID NO: 102) | |
| (forward) | CCACGCACAAGTGATCTACC |
| (SEQ ID NO: 103) | |
| (reverse) | CAAGATGCCTCTATGCCTTAAA |
| Set 3: | |
| (SEQ ID NO: 328) | |
| (forward) | CCACGCACAAGTGATCTACC |
| (SEQ ID NO: 329) | |
| (reverse) | TCCCCTTACATCTGCCACTT |
| Set 4: | |
| (SEQ ID NO: 330) | |
| (forward) | GTGTTCAGGAGCTGGGTGAC |
| (SEQ ID NO: 331) | |
| (reverse) | TCCCCTTACATCTGCCACTT |

The expected size of the PCR product generated by each primer set is as follows: Set 1: 110 basepairs (bp); Set 2: 152 bp; Set 3: 205 bp; Set 4: 225 bp. The primers' specificity was validated by PCR of the genomic sequences. Only primer set 2 consistently amplified products of the expected size (152 nt) in RT-PCR of the small RNA fraction (<200 nt) isolated from various cells. Nested PCR of the 152 nt sequence using primer set 1 also generated products of the expected size (110 nt). The purified PCR products were confirmed by direct sequencing. The sequences of the 152 and 110 nt PCR products are shown below

152 nt sequence:
SEQ ID NO: 332
5′-
CCACGCACAAGTGATCTACCAGTCTTTTAAA[A/G]TTCTATTATTAAAACCCAAACATGCT
CTTTCATTTCCACAGAACACTGGGTCTAAATTTAGACTGGTGCATCCTGATGCTGCACCA
GTCTGCTCTTAATTTAAGGCATACAGGCATCTTG -3′
110 nt sequence:
SEQ ID NO: 333
5′-
CACGCACAAGTGATCTACCAGTCTTTTAAA[A/G]TTCTATTATTAAAACCCAAACATGCTC
TTTCATTTCCACAGAACACTGGGTCTAAATTTAGACTGGTGCATCCTGATGC -3′

A short 52 nucleotide subsequence around the rs2670660 SNP (which did not include other SNPs) was selected for further analysis. The sequence of the 52 nucleotide rs2670660 subsequence used in the biological experiments is SEQ ID NO:1 (see Table 1, infra). As demonstrated by the following experiments, this minimal SNP-containing sequence was biologically active. Without being bound by any particular theory, it is suggested that the minimal 52 nucleotide sequence represents a biologically active splice variant of the longer endogenous RNA sequence and that this small SNP-containing variant is the active species catalyzing the changes in gene transcription that underlie the observed effects of the SNP on disease association.

The following terms are used to designate the 4 small RNAs transcribed from the A-allele of rs2670660, the G-allele of rs2670660, and their antisense counterparts: “A-allele RNA”, “G-allele RNA”, “asA-allele RNA”, and “asG-allele RNA”. These 4 RNAs are also referred to collectively as “the '660 RNAs” or the “rs2670660-encoded small RNAs.” These RNAs may also be referred to herein as NAPL1-locus RNAs or NALP1-lous transRNAs.

Sequence homology profiling and structure/function analyses showed that the '660 RNAs may physically interact with certain miRNAs. The set of miRNAs analyzed was one of those whose expression was found to be modulated by ectopic expression of the '660 RNAs (see below). 36 miRNAs had at least one potential target site within the 152 nt '660 RNA sequence (FIG. 3G). Many miRNA target sites showed allele-associated changes in the minimal free energy (mfe) of hybridization (between the '660 RNA alleleic variant and the miRNA). The miRNAs also share multiple sequence identity segments of at least 11 nucleotides in length with the MEG3 and MALAT1 long non-coding RNAs (FIG. 3G). Comparisons of the allele-associated changes of the mfe values and experimentally-defined changes of the miRNA expression levels revealed a highly significant inverse correlation between these two variables. Lower mfe values correlated with higher levels of miRNA expression (Fig. X). These results suggest a model of snpRNA-mediated regulation of miRNA expression according to which high affinity (low mfe) snpRNA alleles would facilitate increase abundance levels of corresponding microRNAs.

1.4 Expression of rs2670660 Sequence-Bearing Small RNAs Causes Allele-Specific Changes in the Biological Behavior of Cells

A panel of GFP-tagged lentiviral vectors containing allele-specific variants of the rs2670660 sequence under the constitutive expression of the CMV promoter was constructed. The same vector, without the rs2670660 sequences and expressing GFP only, was used as a control (referred to variously in the following and the figures as “vector,” “control,” or “GFP”). The 52 nt allele-specific variants of the rs2670660 sequence were chemically synthesized in sense and anti-sense orientations and cloned into the lentiviral vectors. The sequences were confirmed by restriction mapping and direct sequencing. Preliminary experiments established that hTERT-immortalized BJ1 cells consistently produced the highest transfection efficiency (>90% of GFP-expressing cells by flow cytometry (FACS) analysis). These cells were used for subsequent experiments.

Monolayer Cell Growth and Clonogenic Cell Growth

Monolayer cultures of BJ1 cells expressing 50 nucleotide RNAs from the G-allele of rs2670660 showed reduced growth compared to either cells transfected with the empty GFP vector or cells expressing 50 nucleotide RNAs from the A-allele of rs2670660 (FIG. 4A). Clonogenicity assays demonstrated that cells expressing G-allele RNA and anti-sense A-allele RNA also had markedly reduced clonogenic growth compared to vector control and cells expressing the A-allele RNA (FIG. 4B). In contrast, cells expressing anti-sense G-allele RNA showed increased clonogenic growth. These data indicate that the antisense transcripts are able to antagonize the biological activity of the A- and G-allele transcripts.

Cell Cycle Progression

Fluorescence assisted cell sorting (“FACS”), also referred to herein as “flow cytometry” was used to evaluate the cell-cycle specific effects of these small RNAs. Cells expressing either the anti-sense A (asA) or G-allele (G) showed an increase in the G1 phase and a concomitant decrease in S and G2/M phases. In contrast, cells expressing either the anti-sense G-(asG) or A-allele (A) RNAs showed a decrease in G1 and an increase in S phase (FIG. 4C). These results indicate that the growth inhibitory effects of the asA and G RNAs is associated with G1 arrest while the growth stimulatory effects of asG and A are associated with increased entry into S-phase.

The sequence-specificity of the observed effects on cell growth was tested in a series of allele-combination experiments. In these experiments, cells were co-transfected with lentiviruses expressing complimentary rs2670660 sequences in sense and anti-sense orientations (FIG. 5A-B). Co-expression of asG with G allele RNAs markedly reduced the inhibition of clonogenic growth observed for cells expressing only the G allele RNA (compare top 2 rows of FIG. 5B). Co-expression of A allele RNAs with asA RNAs substantially reduced the growth inhibitory effects of the A-allele RNAs. The simultaneous expression of the G- and asA allele RNAs resulted in the almost complete inhibition of clonogenic growth (FIG. 5B, compare bottom row (row 6 from top) with row 5 (GFP only)). These results further indicate that the growth inhibitory effects of the G-allele RNA and asA allele RNA are sequence specific.

TPA-Induced Differentiation

THP-1 cells undergo differentiation from monocytes to macrophages in response to TPA. Differentiated cells are easily recognized due to their morphological appearance. THP-1 cells expressing the rs2670660-encoded RNAs were identified and sorted by flow cytometry so that cells used for analysis were more than 90% GFP-positive. Cells containing either vector alone (control), A-allele, or G-allele RNAs were exposed to TPA for 4 days. FIG. 6A shows light microscopy (left 3 panels) and fluorescence (right 3 panels) images of cells transfected with vector alone (top 2 panels), A-allele RNA (middle panels), or G-allele RNA (bottom panels). Both the vector-transfected and A-allele expressing cells show a high proportion of cells exhibiting the morphology of the differentiated phenotype. In contrast, G-allele expressing cells failed to differentiate in response to TPA. Instead, the G-allele expressing cells underwent apoptosis during TPA-induced differentiation and as a consequence generated 5-fold fewer macrophages compared to cells expressing the A-allele (FIG. 6B). In contrast, A-allele expressing cells produced nearly 2-fold more macrophages than control cells expressing only GFP. These cells also exhibited more potent phagocytic activity compared to controls or G-allele expressing cells (FIG. 6B, inset). These phenotypic changes were not the result of generally diminished cellular function in the G-allele expressing cells because cells expressing the G-allele showed a sustained long-term viability and increased motility (FIG. 6E).

Cells stably expressing the rs2670660-encoded RNAs were further analyzed for gene expression changes by microarray analysis. The G-allele expressing cells showed lower expression of genes comprising the PRC1-type PcG protein complexes (BMI1 and RING1B) compare to components of the PRC2-type PcG complexes (EZH2, EED, and SUZ12). There was also differential regulation of 586 PcG targeted bivalent chromatin domain genes (see FIG. 6C)

Lentiviral gene transfer was used to (1) inhibit the expression of BMI1 gene in ancestral A-allele-expressing THP-1 cells (using shRNAs) and (2) overexpress the BMI1 gene in pathological G-allele-expressing THP-1 cells. RT-PCR analysis was used to validate the specificity of gene silencing and gene transfer experiments. The cells were assessed for their ability to undergo the differentiation from monocyte to macrophage (FIG. 6D). The BMI1 knock-down markedly diminished macrophage production by A-allele expressing THP-1 cells (FIG. 6D, top and bottom left panels), whereas BMI1 over-expression rescued the macrophage-producing defect of G-allele expressing THP-1 cells (FIG. 6D, bottom right panels).

Further analysis revealed that G-allele expressing cells had pleiotropic deficiencies within the inflammasome/innate immunity pathways. G-allele-associated molecular defects included a concomitant decrease in expression of the NLRP1, CASP1, and IL1-beta genes. These genes are key linear components of an essential functional axis within inflammasome/innate immunity pathway.

Collectively, these data indicate that expression of NALP1-locus transRNAs containing a disease-associated G-allele may cause a significant functional deficiency of the immune system. Markedly enhanced apoptosis during differentiation would reduce the production of specialized immune cells, including effector cells and cells with critical immuno-regulatory functions. Significantly diminished expression of NLRP1, CASP1, and IL1-beta genes would likely severely limit the functional potency of the inflammasome/innate immunity pathways.

1.5 Expression of rs2670660 Sequence-Bearing 50 nt RNAs Causes Genome-Wide Allele-Specific Changes in Gene Expression

Microarray analysis revealed allele-specific changes in the global gene expression profiles of cells expressing the A- and G-allele RNAs of rs2670660 compared to cells expressing the vector alone. Analysis of individual genes showed that expression of the asA- or asG-allele RNA specifically antagonized the expression pattern observed with the corresponding sense allele (FIG. 7A-D).

Microarray analyses revealed genome-wide allele specific concordant and discordant expression profiles in BJ1 cells expressing the rs2670660 RNAs (FIG. 7E-L). Linear regression analysis of the gene expression data was used to graphically illustrate concordant (E-H) and discordant (I-L) expression patterns.

Gene expression that is concordant across tissues is more likely to be influenced by genetic variability than expression that is discordant between tissues. See e.g., French, D. et al., (2008) Concordant Gene Expression in Leukemia Cells and Normal Leukocytes Is Associated with Germline cis-SNPs, PLoS ONE 3(5): e2144. doi:10.1371/journal.pone.0002144. Here, the set of genes that was segregated according to specific concordant and discordant expression profiles demonstrated better sample discrimination (see e.g., FIG. 12A-H, compared to FIG. 12I)

A summary of the concordance analyses is shown in the tables below. In Table 5, a set of 3299 genes whose expression was differentially regulated in cells expressing the G-allele RNA of rs2670660 compared to vector controls was defined by t-statistics. The expression of these 3299 genes was then evaluated in cells expressing the G-allele RNA and in cells expressing the A-allele RNA of rs2670660. Regression analysis shows highly concordant expression of this set of genes in cells expressing the G- and A-allele RNA of rs2670660.87% of the 3299 genes were concordantly expressed (1562 up- and 1732 down-regulated). See also FIG. 7E. Concordance was greater 95% for a subset of genes identified as differentially expressed in cells expressing the G-allele RNA of rs2670660 (at p=0.05) and then evaluated in cells expressing the G-allele RNA and in cells expressing the A-allele RNA of rs2670660 (at p=0.1). See also FIG. 7F. As shown in Table 5, 1,562 genes showed concordant up-regulation in cells expressing the G-allele RNA compared with cells expressing GFP only. When compared to cells expressing the A-allele RNA, 87% showed concordant up-regulation (1,365 out of 1,562).

TABLE 5
Concordance analysis of 3299 and 1561 rs2670660
G-allele RNA-regulated transcripts

| G vs Control | G vs A | G vs Control | G vs A |
|---|---|---|---|
| UP | UP | DOWN | DOWN |
| 1562 | 1365 | 1737 | 1548 |
| Concordance % | 87% | 89% | |
| 834 | 796 | 727 | 695 |
| Concordance | 95% | 96% | |

Concordance for 3299 transcripts identified at cut-off p = 0.050 (for G vs Control) and concordant changes in G vs A samples. Concordance for 1561 transcripts identified at P = 0.050 (for G vs Control) and p = 0.10 (for G vs A).

TABLE 6
Concordance analysis of 3268 and 1636 rs2670660
G-allele RNA-regulated transcripts

| G vs A | G vs Control | G vs A | G vs Control |
|---|---|---|---|
| UP | UP | DOWN | DOWN |
| 1583 | 1428 | 1685 | 1471 |
| Concordance | 90% | 87% | |
| 897 | 875 | 739 | 693 |
| Concordance | 98% | 94% | |

Concordance for 3268 transcripts identified at cut-off p = 0.050 (for G vs A) and concordant changes in G vs Control samples. Concordance for 1636 transcripts identified at P = 0.050 (for G vs A) and p = 0.10 (for G vs Control).

In Table 6, a set of 3,268 genes whose expression was differentially regulated in cells expressing the G-allele compared to cells expressing the A-allele RNA of rs2670660 was defined by t-statistics. The expression of these 3268 genes was then evaluated in cells expressing the G-allele of rs2670660 compared to vector (GFP only) controls. Regression analysis shows highly concordant expression of this set of genes. 89% of 3268 genes were concordantly expressed (1583 up- and 1685 down-regulated). See also FIG. 7G. Concordance was greater than 95% for a subset of 1568 genes identified as differentially expressed in cells expressing the G-allele RNA of rs2670660 (at p=0.05) and then evaluated in cells expressing the G-allele RNA and in cells expressing vector controls (at p=0.1). See also FIG. 7H.

FIGS. 17 and 18 show the complete set of genes identified in the concordance analyses summarized in Tables 5 and 6, respectively. Shown in the figures are the probe set used to measure gene transcription next to the gene expression level (i.e., relative to vector controls for Table 5), the normalized (log 10) gene expression level, and the t-statistic, followed by identification of the gene and alignment used in the analysis.

One set of genes identified as being differentially regulated by the rs2670660 RNAs included the NLRP1, NLRP3, HMGA1, and Myb genes, which are regulators of inflammation and innate immunity (FIG. 8A, top panels). These changes in gene expression are further illustrated by the ratios of the functionally-related transcripts, NLRP3/NLRP1 (FIG. 8A, bottom left panel) and HMGA1/Myb (FIG. 8A, bottom right panel).

The changes in the expression of these genes in human neutrophils after bronchoscopic endotoxin (LPS) challenge (FIG. 8B) and in human leukocytes after in vitro LPS challenge (FIG. 8C, E) was also analyzed. Alveolar neutrophils (FIG. 8B right sets of bars) showed a decreased NLRP1 mRNA expression, increased NLRP3 mRNA expression, and increased NLRP3/NLRP1 mRNA expression ratios compared to the circulating neutrophils (FIG. 8B left sets of bars). LPS-treated leukocytes (FIG. 8C right sets of bars) showed decreased NLRP1 mRNA expression, increased NLRP3 mRNA expression, and increased NLRP3/NLRP1 mRNA expression ratios compared to the control cultures (FIG. 8C left sets of bars). Alveolar neutrophils (FIG. 8D right sets of bars) showed increased Myb mRNA expression, increased HMGA1 mRNA expression, and increased HMGA1/Myb mRNA expression ratios compared to the circulating neutrophils (FIG. 8D left sets of bars). Adherent cultures of monocytes (FIG. 8E, right sets of bars) showed decreased Myb mRNA expression, increased HMGA1 mRNA expression, and increased HMGA1/Myb mRNA expression ratios compared to the control cultures (FIG. 8E left sets of bars).

The set of genes whose expression was differentially regulated in G-allele expressing cells compared to vector (GFP) controls was identified by t-statistics in BJ1 cells. This set was screened for concordance in model systems for activation of the inflammasome pathway activation (FIG. 9). Concordant G-allele signatures were identified in experimental (FIG. 9A, left set of bars) and control (FIG. 9A, right set of bars) samples for human circulating leukocytes after in vitro endotoxin (LPS) challenge. Similar results are shown for human alveolar (FIG. 9B, left set of bars) and circulating neutrophils (FIG. 9B, right set of bars) after in vivo bronchoscopic endotoxin (LPS) challenge. Discordant signatures are shown in panels D and E. Results for human circulating neutrophils after in vivo bronchoscopic endotoxin (LPS) challenge are shown in FIG. 9C, and 9F. Where the gene expression data is not segregated into concordant and discordant groups, diminished sample discrimination is seen (FIG. 9G).

The following tables show the total numbers of genes whose expression changed (either up or down) under various experimental conditions modeling activation of the innate immunity/inflammasome pathways in cells expressing the G-allele RNA of rs2670660 and in control cells expressing only GFP. As shown in the tables, a statistically significant subset of genes regulated by the G-allele RNA of rs2670660 is also differentially regulated when the innate immunity/inflammasome pathways are activated.

TABLE 7
rs2670660-associated gene expression signatures
in transdifferentiating human monocytes

| | Total | UP | UP | DOWN | DOWN |
|---|---|---|---|---|---|
| rs2670660_G_allele | 3299 | 1562 | 1562 | 1737 | 1737 |
| MONOCYTES_UP | | 2269 | | 2269 | |
| MONOCYTES_DOWN | | | 2854 | | 2854 |
| MONOCYTES_TOTAL | 5123 | | | | |
| Common transcripts | 902 | 126 | 326 | 237 | 213 |
| P value | 0 | 6.954E−13 | 0 | 0 | 0 |

TABLE 8
rs2670660-associated gene expression signatures
in LPS-challenged human leukocytes

| | Total | UP | UP | DOWN | DOWN |
|---|---|---|---|---|---|
| rs2670660_G_allele | 3299 | 1562 | 1562 | 1737 | 1737 |
| LEUKOCYTES_UP | | 496 | | 496 | |
| LEUKOCYTES_DOWN | | | 577 | | 577 |
| LEUKOCYTES_TOTAL | 1073 | | | | |
| Common transcripts | 216 | 28 | 80 | 54 | 54 |
| P value | 0 | 0.00032 | 0 | 4.1498E−15 | 1.751E−12 |

TABLE 9
rs2670660-associated gene expression signatures in human
neutrophils after bronchoscopic endotoxin (LPS) challenge

| | Total | UP | UP | DOWN | DOWN |
|---|---|---|---|---|---|
| rs2670660_G_allele | 3299 | 1562 | 1562 | 1737 | 1737 |
| NEUTROPHILS_UP | | 1489 | | 1489 | |
| NEUTROPHILS_DOWN | | | 1565 | 1565 | |
| NEUTROPHILS_TOTAL | 3054 | | | | |
| Common transcripts | 587 | 111 | 120 | 205 | 151 |
| P value | 0 | 0 | 0 | 0 | 0 |

In summary, the allele-specific changes in gene expression in cells expressing the A- and G-allele RNAs of rs2670660 were readily detectable in both in vitro and in vivo models of the activated state of the innate immunity/inflammasome pathways. These results indicate that an rs670660-encoded RNA-driven pathway is activated when innate immunity/inflammasome pathways are activated in a cell.

1.6 rs2670660-Encoded RNAs Affect Expression of MicroRNAs

The genome-wide effects of rs2670660-encoded RNAs on gene expression described above indicate that the specific targets of these RNAs are either transcription factors or miRNAs, both of which control the expression of multiple genes. As discussed above, the predicted secondary structures for many of the identified intergenic small non-coding RNAs also indicated some interaction with miRNAs. Indeed, as demonstrated by the following experiments, the rs2670660 RNAs affect the expression of hundreds of miRNAs and miRNA-targeted proteins.

The effects of the rs2670660-encoded RNAs on the expression of miRNAs was analyzed using an ABI Q-RT-PCR technology platform. The results demonstrated that the rs2670660-encoded RNAs alter the abundance levels of hundreds miRNAs (FIG. 10). Both allele-specific and allele context-independent patterns of miRNA expression were identified. The matching mRNA expression profiles of both the common 140-gene signature (FIG. 10C) and the allele-specific 86-gene miRNA signatures were identified (FIG. 10E). Forced expression of selected individual miRNAs recapitulated both allele context-independent (FIG. 10D) and allele-specific (FIG. 10F) patterns of mRNA expression changes. Interestingly, many mRNAs comprising the 59-gene signature manifest discordant patterns of regulation in response to expression of the control miRNA, miR-205 (right set of bars), expression of which is not altered by rs2670660-encoded RNAs. Also note that miR-20b is one of the up-regulated miRNAs shown in FIG. 10A and mRNAs comprising the 59-gene signature are a sub-set of mRNAs comprising the 140-gene signature shown in FIG. 10C.

Expression profiling experiments also identified 36 miRNAs differentially regulated in BJ1 cells expressing distinct allelic variants of the rs2670660-encoded RNAs (FIG. 10H, I). These represent distinct classes of non-coding RNAs including snoRNAs and snoRNA-host genes (SNORD113; SNHG1; SNHG3; SNHG8); long non-coding RNAs (MEG3, tncRNA, and MALAT1); microRNAs, microRNA-precursors, and protein-coding microRNA-host genes (ATAD2; KIAA1199). 18 of 36 (50%) of these miRNAs are derived from the single miRNA cluster on ˜200 kb continuous region of 14q32 band of chromosome 14, which suggests that the 14q32 cluster miRNAs may be a primary molecular target of the rs2670660-encoded RNAs.

Analysis of genomic coordinates revealed that the sequences encoding 18 of these RNAs are located within about 200 kilobase regions on chromosome 14q32 which is immediately adjacent to the long non-coding RNA gene, MEG3. Changes of expression of intron-residing miRNAs miR-548d (intron of the ATAD2 gene) and miR-549 (intron of the KIAA1199 gene) corresponded to the allele-specific expression levels of corresponding miRNA-host genes, suggesting a coordinated mechanism of regulation. These results indicate that one of the important epigenetic features of the expression of the rs2670660-encoded RNAs is genome-wide changes in expression of multiple diverse classes of non-coding RNAs.

Recent experiments demonstrate that let-7 miRNA release from complexes with Argonaute proteins and subsequent degradation can both be blocked by addition of miRNA target RNA which results in increased levels of let-7 miRNA (Chatterjee et al., Nature 461:546-9, 2009). Computer modeling experiments demonstrated that let-7b miRNA follows the pattern of allele-associated mfe changes characteristic of miRNAs expression levels of which are lower in G-allele expressing cells (FIG. 10J(d)). If the let-7 bioactivity model is valid for the snpRNA-mediated effects on miRNAs, then let-7b expression and activity should be higher in A-allele expressing cells. As shown in FIG. 10J(d), consistent with this, Q-RT-PCR experiments and luciferase reporter assays showed that both expression and activity of the let-7 miRNA are significantly increased in RWPE1 cells stably expressing the A-allele of rs2670660. Similar relationships between snpRNA allele-context-specific mfe changes and effects on miRNA expression and activity were demonstrated for the miR-205 microRNA (FIG. 10J(d), bottom panels). These data suggest that the snpRNAs regulate miRNA abundance and activity in an allele-specific manner by interfering with miRNA release from complexes with Argonaute proteins and preventing subsequent degradation of the miRNA.

A survey of the mRNA targets of the rs2670660-encoded RNAs indicated that rs2670660-associated GES are enriched for genes with an established role in controlling the transition from pluripotency to a differentiated state during development such. For example, rs2670660-associated GES are enriched for genes of loci containing bivalent chromatin domains and PluriNet network genes (FIG. 11A, Table 12). Microarray analysis revealed that expression of rs2670660-encoded RNAs trigger concomitant allele-specific activation of the Polycomb pathway genes (PcG) comprising the Polycomb repressive complex 2 (PRC2). The PRC2 complex catalyzes histone H3 lysine 27 trimethylation (H3K27me3), induces a chromatin silencing state, and mediates transcriptional repression (FIG. 11B).

TABLE 10
Correlation matrix of the rs2670660 allele-specific
effects on expression of 155 PluriNet transcripts

| Pearson | G_allele | A_allele | AS_G_allele | AS_A_alelle |
|---|---|---|---|---|
| G_allele | 1 | 0.2949 | 0.0026 | <0.0001 |
| A_allele | 0.3148 | 1 | <0.0001 | 0.2215 |
| AS_G_Allele | 0.6495 | 0.961 | 1 | 0.0232 |
| AS_A_Alelle | 0.8012 | 0.364 | 0.5177 | 1 |

The table below shows the genes whose expression was regulated by all 4 alleles at a statistical significance of p<0.05. The log-transformed expression values are shown. Positive numbers indicate increased expression, negative numbers indicate decreased expression. Also shown is the primer probe set used in the microarray analysis for each gene.

TABLE 11
140 genes signature of rs2670660 encoded RNAs

| Gene Symbol | G-allele | A-allele | as-A | as-G | Probe Set ID |
|---|---|---|---|---|---|
| TGFB2 | 0.553305756 | 0.702621716 | 0.649238753 | 0.680517363 | 220407_s_at |
| FRMD3 | 0.385526736 | 0.529919499 | 0.597888576 | 0.543488157 | 230645_at |
| ACTC1 | 0.380843293 | 0.647111468 | 0.731352859 | 0.605557138 | 205132_at |
| LOC130576 | 0.322843472 | 0.152573199 | 0.286675549 | 0.356439592 | 228360_at |
| CDCA7 | 0.316566163 | 0.043625367 | 0.221783093 | 0.041490261 | 224428_s_at |
| CTPS | 0.311545801 | 0.222953005 | 0.280567464 | 0.269159808 | 202613_at |
| FRM03 | 0.308398453 | 0.49827964 | 0.592560396 | 0.501013397 | 229893_at |
| TMEM166 | 0.259866362 | 0.064378869 | 0.150617049 | 0.089355051 | 227828_s_at |
| ENC1 | 0.223427966 | 0.257520134 | 0.200001527 | 0.269898418 | 201341_at |
| FGF1 | 0.221428678 | 0.205948066 | 0.145446023 | 0.122946656 | 205117_at |
| CCND3 | 0.208889156 | 0.062540515 | 0.078156651 | 0.085790442 | 201700_at |
| BIRC5 | 0.207779179 | 0.013398897 | 0.087807013 | 0.09970251 | 202095_s_at |
| PDGFA | 0.16577257 | 0.342639247 | 0.200723628 | 0.277345208 | 205463_s_at |
| XYLT1 | 0.145567623 | 0.124948619 | 0.299318377 | 0.098071794 | 213725_x_at |
| LIMCH1 | 0.141859866 | 0.173575546 | 0.217791108 | 0.162585721 | 212325_at |
| PTS | 0.140095976 | 0.10783001 | 0.13874239 | 0.149095145 | 209694_at |
| CFL2 | 0.105747582 | 0.155446177 | 0.127385295 | 0.174312411 | 224352_s_at |
| LIMCH1 | 0.090481672 | 0.089743978 | 0.127811222 | 0.083959267 | 212327_at |
| ATP6V1D | 0.085162444 | 0.059719738 | 0.077641061 | 0.10338699 | 208899_x_at |
| FAM60A | 0.082082206 | 0.220879999 | 0.138002867 | 0.197880426 | 223038_s_at |
| MRPL15 | 0.072819288 | 0.057699647 | 0.073518395 | 0.093478505 | 218027_at |
| MSRB3 | 0.063462145 | 0.133722589 | 0.074775218 | 0.085570288 | 225790_at |
| HSPA4 | 0.052874809 | 0.066549868 | 0.067397189 | 0.111720407 | 211015_s_at |
| PYROXD1 | 0.048059967 | 0.064058397 | 0.041811621 | 0.047633148 | 213878_at |
| HNRNPA2B1 | 0.01968344 | 0.02981391 | 0.058315961 | 0.034502794 | 205292_s_at |
| HDLBP | −0.041944039 | −0.084140658 | −0.102081437 | −0.116195808 | 225012_at |
| GIT2 | −0.053631084 | −0.111610898 | −0.087203395 | −0.106472593 | 225558_at |
| LOC339123 | −0.058738589 | −0.145755985 | −0.087660364 | −0.152420324 | 224886_at |
| CLCN3 | −0.071722517 | −0.044971121 | −0.055262738 | −0.037557857 | 201735_s_at |
| IER2 | −0.074903567 | −0.082073688 | −0.139111162 | −0.085944301 | 202081_at |
| LPAR1 | −0.08101332 | −0.1118548 | −0.107518482 | −0.089766848 | 204036_at |
| SKAP2 | −0.085889261 | −0.065878785 | −0.070110868 | −0.068209214 | 204362_at |
| PIPSK3 | −0.095323013 | −0.079232813 | −0.061956368 | −0.103834526 | 213111_at |
| LITAF | −0.106162554 | −0.052892096 | −0.198219458 | −0.061026308 | 200704_at |
| ARHGAP29 | −0.109176271 | −0.232775427 | −0.124716865 | −0.230615672 | 203910_at |
| UACA | −0.114277207 | −0.241321784 | −0.153292147 | −0.213627077 | 238868_at |
| ANGEL2 | −0.120781462 | −0.068002722 | −0.081381109 | −0.031982546 | 221825_at |
| HLA-E | −0.122609583 | −0.108751082 | −0.147654981 | −0.122137868 | 200904_at |
| SYPL2 | −0.123685453 | −0.158407521 | −0.261306819 | −0.160524265 | 230611_at |
| RHBDF1 | −0.124688289 | −0.100577985 | −0.136247693 | −0.152358562 | 218686_s_at |
| THSD4 | −0.12835456 | −0.23582487 | −0.228870445 | −0.270832845 | 222835_at |
| LTBP1 | −0.136108846 | −0.343215277 | −0.220677181 | −0.377883801 | 202729_s_at |
| TMTC1 | −0.13628934 | −0.249935537 | −0.530211661 | −0.270607072 | 224397_s_at |
| GM2A | −0.139099929 | −0.165685042 | −0.12974658 | −0.141293067 | 212737_at |
| LOXL4 | −0.144847229 | −0.391067218 | −0.373166559 | −0.396202402 | 227145_at |
| WARS | −0.145809709 | −0.091845534 | −0.226796149 | −0.140677555 | 200629_at |
| PCOLCE | −0.158255246 | −0.111705115 | −0.166851931 | −0.176628276 | 202465_at |
| ADAMTS1 | −0.164664241 | −0.078457919 | −0.13127296 | −0.116633384 | 222162_s_at |
| MXRAS | −0.165569027 | −0.306068049 | −0.179399422 | −0.329737541 | 209596_at |
| LGALS3 | −0.166084214 | −0.146887297 | −0.244660644 | −0.173752204 | 208949_s_at |
| SH2133 | −0.170288769 | −0.169453101 | −0.181376414 | −0.163217746 | 203320_at |
| CD109 | −0.178414128 | −0.257304861 | −0.138207625 | −0.216011205 | 226545_at |
| MYST4 | −0.180653527 | −0.176213832 | −0.16419067 | −0.212444796 | 212462_at |
| FKBP7 | −0.194588131 | −0.116507464 | −0.152596349 | −0.135888553 | 224002_s_at |
| FYCO1 | −0.195989945 | −0.170536499 | −0.131735682 | −0.18845219 | 218204_s_at |
| ClOorf116 | −0.200763405 | −0.237996752 | −0.11990838 | −0.133387792 | 203571_s_at |
| EDEM2 | −0.201015215 | −0.125488319 | −0.090584368 | −0.102622565 | 218282_at |
| PTN | −0.205914665 | −0.272314433 | −0.195315996 | −0.395272484 | 209466_x_at |
| GPR177 | −0.209449532 | −0.232599619 | −0.255958883 | −0.256539927 | 228950_s_at |
| SNHG8 | −0.221585573 | −0.122089305 | −0.110762343 | −0.148327784 | 225220_at |
| NISCH | −0.22277806 | −0.101198191 | −0.133005859 | −0.18482463 | 201591_s_at |
| GPR177 | −0.226413922 | −0.264117701 | −0.287248004 | −0.264718163 | 221958_s_at |
| LOC255480 | −0.227362546 | −0.123015387 | −0.146875114 | −0.146317429 | 233947_s_at |
| TMEM200A | −0.230160096 | −0.32216685 | −0.280217849 | −0.224662502 | 234994_at |
| IF116 | −0.233065276 | −0.105769743 | −0.138895839 | −0.11918646 | 208966_x_at |
| LY6E | −0.241115817 | −0.291525343 | −0.264823796 | −0.308663401 | 202145_at |
| ALDH6A1 | −0.24301583 | −0.135470291 | −0.151397423 | −0.132158974 | 221588_x_at |
| Clorf25 | −0.248525359 | −0.118843104 | −0.136393488 | −0.138151819 | 220992_s_at |
| SPHKAP | −0.249133987 | −0.539616856 | −0.130601742 | −0.481739237 | 228509_at |
| SYTL2 | −0.249692865 | −0.061933787 | −0.21341683 | −0.073876586 | 232914_s_at |
| PTN | −0.250726775 | −0.271291845 | −0.213944492 | −0.38148165 | 211737_x_at |
| 235964_x_at | −0.255279894 | −0.300173347 | −0.225376483 | −0.265563314 | 235964_x_at |
| GSTA4 | −0.258679435 | −0.114179873 | −0.212397 | −0.118753307 | 202967_at |
| NBL1 | −0.270001354 | −0.223233736 | −0.234999802 | −0.35222198 | 201621_at |
| 228304_at | −0.271979764 | −0.190927055 | −0.202928834 | −0.245810444 | 228304_at |
| DCN | −0.273382984 | −0.196286376 | −0.311783823 | −0.36715458 | 211896_s_at |
| CASP1 | −0.275627594 | −0.083509781 | −0.193889294 | −0.079402796 | 211366_x_at |
| GPR177 | −0.277547099 | −0.274571429 | −0.293684369 | −0.262060515 | 228949_at |
| C20orf108 | −0.294126197 | −0.108745061 | −0.174985691 | −0.164504239 | 224690_at |
| S1PR3 | −0.305709745 | −0.282841272 | −0.391065837 | −0.237382091 | 228176_at |
| KCNN2 | −0.313473154 | −0.306633655 | −0.176472666 | −0.247951177 | 220116_at |
| SH3BPS | −0.315379344 | −0.225774418 | −0.32423095 | −0.237687388 | 201811_x_at |
| M EST | −0.321386514 | −0.261666357 | −0.502736683 | −0.254505309 | 202016_at |
| LGALS3BP | −0.326305367 | −0.196440026 | −0.339831466 | −0.322145313 | 200923_at |
| PARP14 | −0.327889734 | −0.299551929 | −0.275013294 | −0.306895222 | 224701_at |
| P2RY5 | −0.329770344 | −0.335250433 | −0.348114575 | −0.336633509 | 218589_at |
| AFF3 | −0.334210344 | −0.326687208 | −0.334077536 | −0.316736485 | 227198_at |
| TSHZ1 | −0.336223774 | −0.240827247 | −0.239287462 | −0.262266661 | 223283_s_at |
| SATB1 | −0.34437247 | −0.140774231 | −0.193391908 | −0.173417326 | 203408_s_at |
| SEMA6D | −0.353531103 | −0.355914928 | −0.304132586 | −0.313932992 | 226492_at |
| PBX1 | −0.354524035 | −0.192016854 | −0.189135372 | −0.252346893 | 212148_at |
| IL1R1 | −0.359365758 | −0.118271624 | −0.255671452 | −0.204833791 | 202948_at |
| ORAI3 | −0.360502813 | −0.214779854 | −0.258411374 | −0.206146014 | 221864_at |
| EGR1 | −0.360631747 | −0.394991843 | −0.512704384 | −0.560313954 | 201693_s_at |
| GREM2 | −0.366978506 | −0.222450104 | −0.187213711 | −0.201161571 | 235504_at |
| TSHZ1 | −0.367453904 | −0.195771558 | −0.21740912 | −0.235421537 | 223282_at |
| PTGS1 | −0.376490271 | −0.189678649 | −0.267370997 | −0.243708837 | 205128_x_at |
| PSD3 | −0.397512786 | −0.250138877 | −0.340415108 | −0.282459269 | 203355_s_at |
| UST | −0.407263816 | −0.103182821 | −0.197596707 | −0.100621952 | 205139_s_at |
| I FITM1 | −0.407333309 | −0.25431946 | −0.267349793 | −0.226894331 | 201601_x_at |
| ANGPTL2 | −0.409644223 | −0.288322174 | −0.363539973 | −0.350947748 | 213004_at |
| PTGS1 | −0.416442992 | −0.223128977 | −0.30911195 | −0.279577181 | 215813_s_at |
| EGR1 | −0.421782615 | −0.396985742 | −0.556843988 | −0.564568462 | 227404s_at |
| 235938_at | −0.424746088 | −0.257164947 | −0.207686418 | −0.256438653 | 235938_at |
| C6orf32 | −0.425765079 | −0.132125002 | −0.399418076 | −0.250368924 | 209829_at |
| EGR1 | −0.428738974 | −0.412355045 | −0.544074133 | −0.526444237 | 201694_s_at |
| APCDD1 | −0.428909441 | −0.154201749 | −0.250672619 | −0.289535379 | 225016_at |
| ROBO2 | −0.435339507 | −0.388406475 | −0.487914817 | −0.488346059 | 226766_at |
| ENPP2 | −0.440809764 | −0.203154032 | −0.4502019 | −0.200260001 | 209392_at |
| ZNF521 | −0.443326893 | −0.33946231 | −0.42069622 | −0.423202751 | 226677_at |
| SALL2 | −0.444384522 | −0.34117693 | −0.228403707 | −0.603214367 | 213283_s_at |
| EFEMP1 | −0.447324597 | −0.249817275 | −0.480613724 | −0.385913963 | 201843_s_at |
| CLEC3B | −0.453130908 | −0.365252025 | −0.441731159 | −0.525097001 | 205200_at |
| PTPRN2 | −0.47167725 | −0.190605828 | −0.746619742 | −0.846145731 | 203030_s_at |
| EFEMP1 | −0.47938935 | −0.262282887 | −0.447117294 | −0.397455846 | 201842_s_at |
| DKFZP586H2123 | −0.490972116 | −0.444586187 | −0.346902728 | −0.403255234 | 213661_at |
| MASP1 | −0.491471632 | −0.157997344 | −0.349133341 | −0.227803704 | 232224_at |
| 234222_at | −0.502752499 | −0.714453247 | −0.756512708 | −0.790724876 | 234222_at |
| 233059 _at | −0.504230965 | −0.353128428 | −0.263836738 | −0.419528194 | 233059_at |
| LOC221091 | −0.508630176 | −0.386822499 | −0.506264216 | −0.551338464 | 1556427_s_at |
| C1S | −0.513840527 | −0.120778792 | −0.298883493 | −0.305752762 | 208747_s_at |
| PRSS12 | −0.521871037 | −0.310069564 | −0.305268732 | −0.411157562 | 205515_at |
| IFI6 | −0.524744486 | −0.24896657 | −0.210325648 | −0.261347724 | 204415_at |
| ARMC9 | −0.539799784 | −0.313851343 | −0.239889191 | −0.262112666 | 219637_at |
| ARMC9 | −0.548744533 | −0.212803041 | −0.141221217 | −0.182792057 | 219636_s_at |
| ANGPTL2 | −0.571582031 | −0.333095185 | −0.46101918 | −0.441252306 | 213001_at |
| RGS2 | −0.607252174 | −0.490377927 | −0.570291247 | −0.593940447 | 202388_at |
| SLC29A2 | −0.640001535 | −0.431008409 | −0.746330564 | −0.658033571 | 1560062_at |
| LXN | −0.640499395 | −0.080952232 | −0.423504977 | −0.15451491 | 218729_at |
| STC1 | −0.660872377 | −0.414266124 | −0.602166058 | −0.479604979 | 230746_s_at |
| 234748_x_at | −0.678880772 | −0.815810296 | −0.760124031 | −0.708161548 | 234748_x_at |
| SERPINF1 | −0.679905991 | −0.313772618 | −0.575209027 | −0.570912636 | 202283_at |
| TMEM119 | −0.696042321 | −0.318826134 | −0.46962944 | −0.415490533 | 227300_at |
| C13orf15 | −0.705486248 | −1.011744056 | −1.115383874 | −0.852870973 | 218723_s_at |
| 1559478_at | −0.712227696 | −0.509683087 | −0.675999067 | −0.651715887 | 1559478_at |
| STC1 | −0.726500644 | −0.460933297 | −0.627508711 | −0.539513289 | 204595_s_at |
| EYA1 | −0.753409599 | −0.444524151 | −0.59282689 | −0.629432578 | 214608_s_at |
| CLDN11 | −0.932173728 | −0.967850056 | −1.065196888 | −0.951635607 | 228335_at |
| OR12D3/OR5V1 | −0.942753756 | −0.691382096 | −0.804177631 | −1.041767239 | 208098_at |
| CD4 | −0.94677462 | −0.759531119 | −0.914076809 | −1.073136515 | 216424_at |

Correlation matrix for the 140 gene signature

| | G allele | A allele | AS_A | AS_G |
|---|---|---|---|---|
| G allele | 1 | <0.0001 | <0.0001 | <0.0001 |
| A allele | 0.851355013 | 1 | <0.0001 | <0.0001 |
| AS_A | 0.905274554 | 0.919669399 | 1 | <0.0001 |
| AS_G | 0.891048722 | 0.94446759 | 0.943972803 | 1 |

1.7 Clinical Relevance of Allele-Specific Effects on Gene Transcription by rs2670660-Encoded Trans-Regulatory RNAs

These microarray gene expression profiling results discussed above were expanded to analyze the effects of the expression of the rs2670660 encoded RNAs in other cell types and experimental systems as detailed in the table below. In each of these experimental systems, there was statistically significant evidence of the activation of rs2670660-associated gene expression signatures. The table below shows the spectrum of common human diseases and types of clinical samples analyzed by microarray gene expression profiling.

TABLE 12
Patient samples analyzed by microarray gene expression profiling.
Abbreviations: PBMC, peripheral blood mononuclear cells. List
of GEO accession numbers and original references for microarray
analyses and associated clinical information can be found in
references listed in Materials and Methods.

| | No. | |
|---|---|---|
| Disease State | patients | Sample type |
| Control | 14 | PBMC |
| Alzheimer's | 14 | PBMC |
| Control | 9 | Brain hippocampi from 9 control subjects |
| Alzheimer's | 22 | Brain hippocampi from 22 postmortem |
| | | subjects with Alzheimer's disease (AD) |
| Control | 15 | Lymphoblastoid cells |
| Autism | 15 | Lymphoblastoid cells |
| Control | 42 | PBMC |
| Crohn's disease | 59 | PBMC |
| Ulcerative colitis | 26 | PBMC |
| Control | 11 | PBMC |
| Rheumatoid arthritis | 20 | PBMC |
| Control (lean) | 14 | Cultured abdominal subcutaneous |
| | | preadipocytes |
| Obesity | 14 | Cultured abdominal subcutaneous |
| | | preadipocytes |
| Control | 8 | Normal breast tissues |
| Breast cancer | 99 | Primary & metastatic breast cancer |
| | | tissues |
| Breast cancer | 8 | Normal breast tissue of patients with |
| | | metastatic breast cancer |
| Breast cancer | 26 | lymph node of patients with metastatic |
| | | breast cancer |
| Breast cancer | 12 | Distant metastatic breast cancer tissues |
| Control | 18 | Normal prostate tissues |
| Prostate cancer | 64 | Primary & metastatic prostate cancer |
| | | tissues |
| Prostate cancer | 62 | Normal prostate tissue adjacent to tumor |
| Prostate cancer | 25 | Distant meetastatic prostate cancer |
| | | tissues |
| Control | 14 | PBMC |
| Huntington disease | 17 | PBMC |
| Control | 6 | Leukocytes |
| LPS challenge | 6 | Leukocytes |
| Control | 3 | Primary human monocytes |
| Transdifferentiation | 6 | Primary human monocytes |
| Control | 14 | Circulating neutrophils |
| Bronchoscopic LPS | 17 | Circulating neutrophils |
| challenge | | |
| Bronchoscopic LPS | 17 | Alveolar neutrophils |
| challenge | | |
| Samples | 697 | |
| Control Subjects | 185 | |
| Patients | 350 | |

The following tables show the total numbers of genes differentially expressed in clinical samples of diseased tissues compared to matched healthy tissues and concordance with the set of genes differentially regulated by the G-allele RNA of rs2670660. As shown in the tables, a statistically significant subset of genes regulated by the G-allele RNA of rs2670660 is also differentially regulated in various diseased tissues.

TABLE 13
rs2670660-associated Crohn's disease
(CD) gene expression signatures

| | Total | DOWN | DOWN | UP | UP |
|---|---|---|---|---|---|
| rs2670660_G_Allele | 3299 | 1737 | 1737 | 1562 | 1562 |
| CD PBMC_UP | | | 2582 | | 2582 |
| CD PBMC_DOWN | | 3362 | | 3362 | |
| CD PBMC_TOTAL | 5944 | | | | |
| COMMON TRANSCRIPTS | 1072 | 281 | 304 | 336 | 151 |
| P VALUE | 0 | 0 | 0 | 0 | 0 |

TABLE 14
rs2670660-associated rheumatoid arthritis
(RA) gene expression signatures

| | Total | DOWN | DOWN | UP | UP |
|---|---|---|---|---|---|
| rs2670660_G_Allele | 3299 | 1737 | 1737 | 1562 | 1562 |
| RA PBMC_UP | | | 670 | | 670 |
| RA PBMC_DOWN | | 1971 | | 1971 | |
| RA PBMC_TOTAL | 2641 | | | | |
| COMMON | 489 | 211 | 54 | 184 | 40 |
| TRANSCRIPTS | | | | | |
| P VALUE | 0 | 0 | 4.3E−10 | 0 | 7.3E−06 |

TABLE 15
rs2670660-associated Huntinston's
disease (HD) gene expression signatures

| | Total | UP | UP | DOWN | DOWN |
|---|---|---|---|---|---|
| rs2670660_G_allele | 3299 | 1562 | 1562 | 1737 | 1737 |
| HD_UP | | 2029 | | 2029 | |
| HD_DOWN | | | 1504 | | 1504 |
| HD_TOTAL | 3533 | | | | |
| Common transcripts | 700 | 167 | 135 | 242 | 156 |
| P value | 0 | 0 | 0 | 0 | 0 |

TABLE 16
rs2670660-associated autism gene expression signatures

| | Total | UP | UP | DOWN | DOWN |
|---|---|---|---|---|---|
| rs2670660_G_allele | 3299 | 1562 | 1562 | 1737 | 1737 |
| Autism_UP | | 226 | | 226 | |
| Autism_DOWN | | | 438 | | 438 |
| Autism_TOTAL | 664 | | | | |
| Common transcripts | 79 | 7 | 24 | 15 | 33 |
| P value | 4.49191E−09 | 0.14825 | 0.001092 | 0.003585 | 3.44537E−06 |

TABLE 17
rs2670660-associated metastatic prostate cancer
(PC_METS) gene expression signatures

| | Total | DOWN | DOWN | UP | UP |
|---|---|---|---|---|---|
| rs2670660_G_Allele | 3299 | 1737 | 1737 | 1562 | 1562 |
| PC_METS_UP | | | 3009 | | 3009 |
| PC_METS_DOWN | | 2432 | | 2432 | |
| PC_METS_TOTAL | 5441 | | | | |
| COMMON TRANSCRIPTS | 995 | 334 | 223 | 150 | 288 |
| P VALUE | 0 | 0 | 0 | 0 | 0 |

TABLE 18
rs2670660-associated Alzheimer's
(ALZH) gene expression signatures

| | Total | DOWN | DOWN | UP | UP |
|---|---|---|---|---|---|
| rs2670660_G_Allele | 3299 | 1737 | 1737 | 1562 | 1562 |
| ALZH | | | 1032 | | 1032 |
| BRAIN_UP | | | | | |
| ALZH | | 823 | | 823 | |
| BRAIN_DOWN | | | | | |
| ALZH | 1855 | | | | |
| BRAIN_TOTAL | | | | | |
| COMMON | 304 | 60 | 103 | 76 | 65 |
| TRANSCRIPTS | | | | | |
| P VALUE | 0 | 2.114E−09 | 0 | 0 | 2.31E−09 |

TABLE 19
rs2670660-associated obesity (OB) gene expression signatures

| | Total | DOWN | DOWN | UP | UP |
|---|---|---|---|---|---|
| rs2670660_G_Allele | 3299 | 1737 | 1737 | 1562 | 1562 |
| OBESITY_UP | | | 708 | | 708 |
| OBESITY_DOWN | | 799 | | 799 | |
| OBESITY_TOTAL | 1507 | | | | |
| COMMON | 305 | 111 | 59 | 75 | 60 |
| TRANSCRIPTS | | | | | |
| P VALUE | 0 | 0 | 1.91E−11 | 0 | 8.67E−14 |

TABLE 20
Expression signatures of hESC bivalent domain
genes (BDG) in rs2670660 G-allele-associated
gene expression models of human diseases

| Disease state | Total genes | Down | Up |
|---|---|---|---|
| prostate cancer | 995 | 484 | 511 |
| Prostate cancer | 149 | 97 | 52 |
| BDGs | | | |
| p value | 8.1971e−07 | 7.667E−11 | 0.050813 |
| Percent BDGs | 15 | 20 | 10 |
| Autism | 79 | 47 | 22 |
| Autism BDGs | 9 | 6 | 3 |
| p value | 0.14083503 | 0.1612361 | 0.224763 |
| Percent BDGs | 11 | 13 | 14 |
| Alzheimer's disease | 304 | 136 | 168 |
| Alzheimer's BDGs | 39 | 21 | 18 |
| p value | 0.04177597 | 0.0266486 | 0.100837 |
| Percent BDGs | 13 | 15 | 11 |
| Crohn's disease | 1072 | 617 | 455 |
| Crohn's BDGs | 125 | 46 | 79 |
| p value | 0.03305136 | 0.0003247 | 3.38E−06 |
| Percent BDGs | 12 | 7.4 | 17 |
| Rheumatoid | 489 | 395 | 94 |
| arthritis | | | |
| Rheumatoid | 60 | 35 | 25 |
| arthritis BDGs | | | |
| p value | 0.03796995 | 0.0244844 | 1.05e−05 |
| Percent BDGs | 12.3 | 8.9 | 27 |
| Obesity | 305 | 186 | 119 |
| Obesity BDGs | 65 | 42 | 23 |
| p value | 1.5951e−08 | 1.364E−06 | 0.002381 |
| Percent BDGs | 21 | 23 | 19 |
| Centenerians/Ageing | 229 | 199 | 30 |
| Cemtenerians BGDs | 14 | 4 | 10 |
| p value | 0.0034485 | 7.484e−07 | 0.000717 |
| Percent BDGs | 6.1 | 2.0 | 33 |

It has been reported that activated state of the innate immunity/inflammasome pathways in patients with Crohn's disease and rheumatoid arthritis is associated with altered expression of the NLRPI, NLRP3, HMGA1, and Myb genes which is reflected in altered NLRP3/NLRP1 and HMGA1/Myb mRNA expression ratios. Clinical samples from patients diagnosed with a broad spectrum of disorders associated with activation of these pathways were analyzed for expression of the genes identified in the global gene expression profiles of cells expressing the A- and G-allele RNAs of rs2670660. The set of genes whose expression is altered in cells expressing SNP-associated small RNA molecules is referred to herein as a gene expression signature (“GES”). Thus, the sets of genes whose expression was altered in cells expressing the small RNAs of rs2670660 are referred to as rs2670660-associated allele-specific GES. Specifically, there are four rs2670660-associated allele-specific GES, namely, the signatures of the A-allele, the G-allele, the antisense-A, or antisense-G allele.

Patient samples of peripheral blood mononuclear cells (PBMC) and diseased tissues were analyzed for the rs2670660-associated allele-specific GES by microarray gene expression analysis. rs2670660-associated allele-specific GES were detected with a level of statistical significance that markedly exceeded the probability of random co-occurrence by chance alone in clinical samples from patients diagnosed with Crohn's disease, rheumatoid arthritis, Huntington's disease, and Alzheimer's disease (FIG. 12). GES associated with the expression of the G-allele-specific 52 nt small RNAs in BJ1 cells was identified in clinical samples using t-statistics and screened for concordant and discordant features in corresponding clinical settings to segregate G-allele 46 concordant and G-allele discordant signatures. The assessment of rs2670660-associated allele-specific GES in these clinical samples indicates that the GES are detectable in about 80-100% of samples from patients diagnosed with one of several common diseases manifested by activation of the innate immunity/inflammasome pathways. These data indicate that assays for rs2670660-associated GES may be useful diagnostic and prognostic tools for diseases and disorders characterized by activation of these pathways.

The ability of GES associated with the expression of rs2670660-encoded small RNAs to discriminate normal and pathological tissue samples was further validated in a set of patients with Alzheimer's disease, prostate cancer, and breast cancer (FIG. 13). The set of genes whose expression was differentially regulated by ectopic expression of the rs2670660 G-allele RNA was identified in BJ1 cells using t-statistics. This set of genes was then screened for concordant and discordant expression in clinical samples and matched controls (see Table 13, supra). Expression profiles of G-allele concordant and G-allele discordant signatures in individual samples of each data set were evaluated by calculating Pearson correlation coefficients (signature scores) using the log 10-transformed fold expression changes of G-allele-specific GES in BJ1 cells as a multidimensional standard vector.

FIG. 13A shows the expression profiles of G-allele concordant (left panel) and discordant (right panel) genes in hippocamal tissue from Alzheimer's patients and normal subjects. Each bar represents the G-allele-specific GES for a particular subject calculated as described above. In each panel, the group of 9 bars on the far left shows the GES from tissue in each of 9 control subjects. The next three groups of bars in each panel represent the GES of tissue from Alzheimer's patients segregated based on the clinically-defined severity of the disease, left to right: incipient (7 subjects), moderate (8 subjects), and severe (7 subjects), for a total of 22 subjects. The data show distinct expression profiles in the tissues from Alzheimer's patients versus controls, indicating that these GES can differentiate between normal and diseased tissue with high statistical significance.

FIG. 13B shows the expression profiles of G-allele concordant (left panel) and discordant (right panel) genes in normal and prostate cancer tissues. Each bar represents the G-allele-specific GES for a particular subject calculated as described above. In each panel, the group of 18 bars on the far left shows the GES from normal prostate tissue in each of 18 control subjects. The next three groups of bars in each panel represent the GES of prostate cancer tissues segregated based on histological examination (left to right): morphologically normal prostate tissues adjacent to tumor (62 samples); primary prostate tumors (64); metastatic prostate tumors in distant organs (25). The data show distinct expression profiles, particularly for the metastatic tumors, compared to controls and morphologically normal tissues adjacent to tumor tissue. These data demonstrate that the G-allele GES, segregated into concordant and discordant expression groups, can differentiate between normal and metastatic tumor tissue with high statistical significance.

FIG. 13C shows the expression profiles of G-allele concordant (left panel) and discordant (right panel) genes in normal and breast cancer tissues. Each bar represents the G-allele-specific GES for a particular subject calculated as described above. In each panel, the group of 8 bars on the far left shows the GES from normal breast tissue. The next five groups of bars in each panel represent the GES of breast cancer tissues segregated based on histological examination as follows (left to right): morphologically normal breast tissues adjacent to tumor (8 samples); primary breast tumors from patients without metastatic disease; primary breast tumors from patients with metastatic disease (99 total for primary tumors); lymph nodes from patients with metastatic disease (26); metastatic breast tumors in distant organs (12). The data show distinct expression profiles, particularly for the metastatic tumors, compared to controls and morphologically normal tissues adjacent to tumor tissue. These data demonstrate that the G-allele GES, segregated into concordant and discordant expression groups, can differentiate between normal and metastic tumor tissue with high statistical significance.

The above data show the ability of the gene expression signatures of the G-allele RNA to discriminate between diseased and normal tissues in Crohn's disease, rheumatoid arthritis, Huntington's disease, Alzheimer's disease, breast cancer, and prostate cancers (FIGS. 12, 13, Table 12). Several GES were also identified, using the same protocols as described above, to discriminate between autistic and control subjects using gene expression from lymphoblastoid cells (Table 12, FIG. 14A). A 36-gene signature was particularly useful in discriminating between autistic and control subjects. In addition, a 133-gene G-allele concordant signature was identified using preadipocytes from lean and obese subjects that was able to effectively discriminate between these two groups (Table 12, FIG. 14B). A further 112-gene G-allele discordant signature was also identified that could distinguish obese from lean subjects (FIG. 14C).

The data presented in FIGS. 12-14 indicate that the activated states of the innate immunity/inflammasome pathways (as evidenced by rs2670660-associated GES, see FIGS. 8, 9, 11) are readily detectable in pathology-affected tissues of patients with Crohn's disease, rheumatoid arthritis, Huntington's disease, Alzheimer's disease, breast cancer, prostate cancer, autism, and obesity. Accordingly, the rs2670660-associated GES identified here provide useful research and diagnostic tools for studying and detecting these disease states in tissue from human subjects.

The data presented here demonstrate that intergenic small regulatory RNAs represent a prevalent class of transcripts containing SNP variants associated with common human disorders (FIG. 15A, Tables 21, 22). The data also show that these small RNAs display cell-type specific patterns of expression in human cells (FIG. 1; FIG. 15B, C). This is in contrast to the expression of long non-coding RNAs containing the small RNAs described here. As shown in FIGS. 15B and 15C, the long non-coding RNAs are expressed nearly ubiquitously among cells of mesenchymal (BJ1), lymphoid (U937), and epithelial (RWPE1) origin. This suggests a model of cell type-specific biogenesis of these small non-coding RNA molecules based on differentiation-associated processing of the long non-coding RNAs.

In summary, the data presented here indicate a role for these small non-coding RNAs transcribed from disease-linked SNPs (such the rs2670660-encoded RNAs) in epigenetic reprogramming during development, clonal specialization, and differentiation, as well as during disease progression.

TABLE 21
Small non-coding RNAs and associated long non-coding RNAs
containing SNP sequences expressed in human cells. Molecular
identities of listed non-coding small RNAs were validated
by sequencing of the purified PCR products.

| | No. non- | |
|---|---|---|
| | coding long | |
| | and small | |
| | (parenthesis) | |
| SNP-linked Disease | RNAs | SNP sequence |
| Autoimmune thyroid | 1 (1) | rs10186922 |
| disease | | |
| Alzheimer's | 1 (1) | rs11159647 |
| disease | | |
| Bipolar disorder | 3 (2) | rs6458307; rs2609653; |
| | | rs7570682; |
| Breast cancer | 6 (2) | rs13281615; rs672888; |
| | | rs889312; rs2822558; |
| | | rs13387042; rs2291533 |
| Coronary Artery | 7 (6) | rs1333049; rs2383206; |
| Disease | | rs10757274; rs2383207; |
| | | rs383830; rs7250581; |
| | | rs10757278 |
| Colorectal cancer | 7 (6) | rs16892766; rs7014346; |
| | | rs10505477; rs10808556; |
| | | rs6983267; rs4779584; |
| | | rs10795668 |
| Crohn's | 13 (8) | rs6596075; rs9469220; |
| Disease | | rs2542151; rs10733113; |
| | | rs10883365; rs10761659; |
| | | rs17234657; rs55646866; |
| | | rs6672995; ss107635144; |
| | | rs12037606; rs6601764; |
| | | rs7807268 |
| Hypertension | 3 (1) | rs1937506; rs2820037; |
| | | rs6997709 |
| Multiple Sclerosis | 1 (0) | rs6957669 |
| Ovarian Cancer | 3 (3) | rs10505477; rs10808556; |
| | | rs6983267 |
| Obesity | 1 (1) | rs17782313 |
| Prostate Cancer | 13 (11) | rs10090154; rs1447295; |
| | | rs16901979; rs4242382; |
| | | rs6983561; rs7000448; |
| | | rs7017300; rs7837688; |
| | | rs10505477; rs10808556; |
| | | rs6983267; rs983085; |
| | | rs1859962 |
| Rheumatoid | 5 (3) | rs615672; rs6457617; |
| Arthritis | | rs6679677; rs6920220; |
| | | rs11761231; |
| Schizophrenia | 3 (2) | rs952477; rs12141187; |
| | | rs4132958 |
| Systemic Lupus | 2 (2) | rs10798269; rs729302 |
| Erythematosus | | |
| Type 1 Diabetes | 5 (3) | rs9270986; rs2544677; |
| | | rs2542151; rs6679677; |
| | | rs11171739 |
| Type 2 Diabetes | 9 (7) | rs9472138; rs17705177; |
| | | rs5015480; rs7020996; |
| | | rs10490072; rs1153188; |
| | | rs13071168; rs358806; |
| | | rs7659604 |
| Ulcerative colitis | 1 (0) | rs660895 |
| Vitiligo | 3 (3) | rs2670660; rs2733359; |
| | | rs8182354 |
| Total | 87 (62) | |

TABLE 22
Classification of SNPs associated with common human disorders.

| | | | Chromo- |
|---|---|---|---|
| | | | somal |
| Disease | SNP | SNP Class | Location |
| Azheimer's | rs2573905 | Intronic | X |
| Azheimer's | rs11159647 | Intergenic | 14 |
| Azheimer's/Coronary | rs4420638 | Intronic | 19 |
| Artery Diseases | | | |
| Azheimer's | rs5984894 | Intronic | X |
| Autism | rs17236239 | Intronic | 7 |
| Autism | rs7794745 | Intronic | 7 |
| Lung Cancer | rs8034191 | Intronic | 15q25.1 |
| Lung Cancer | rs2036534 | Intronic | 15q25.1 |
| Lung Cancer | rs1051730 | cds-synon | 15q25.1 |
| Lung Cancer | rs8042374 | Intronic | 15q25.1 |
| Prostate Cancer | rs16901979 | Intergenic | 8q24 |
| Prostate Cancer | rs6983561 | Intergenic | 8q24 |
| Prostate/Colorectal/ | rs6983267 | Intergenic | 8q24 |
| Ovarian Cancer | | | |
| Prostate Cancer | rs7000448 | Intergenic | 8q24 |
| Prostate Cancer | rs1447295 | Intergenic | 8q24 |
| Prostate Cancer | rs4242382 | Intergenic | 8q24 |
| Prostate Cancer | rs7017300 | Intergenic | 8q24 |
| Prostate Cancer | rs10090154 | Intergenic | 8q24 |
| Prostate Cancer | rs7837688 | Intergenic | 8q24 |
| Prostate/Colorectal/ | rs10505477 | Intergenic | 8q24 |
| Ovarian Cancer | | | |
| Prostate/Colorectal/ | rs10808556 | Intergenic | 8q24 |
| Ovarian Cancer | | | |
| Breast Cancer | rs13281615 | Intergenic | 8q24 |
| Breast Cancer | rs672888 | Intergenic | 8q24 |
| Colorectal Cancer | rs10795668 | Intergenic | 10 |
| Colorectal Cancer | rs16892766 | Intergenic | 8 |
| Colorectal Cancer | rs3802842 | Intronic | 11 |
| Colorectal Cancer | rs4779584 | Intergenic | 15 |
| Colorectal Cancer | rs4939827 | Intronic | 18 |
| Prostate/Colorectal/ | rs6983267 | Intergenic | 8 |
| Ovarian Cancer | | | |
| Prostate/Colorectal/ | rs10505477 | Intergenic | 8q24 |
| Ovarian Cancer | | | |
| Prostate/Colorectal/ | rs10808556 | Intergenic | 8q24 |
| Ovarian Cancer | | | |
| Colorectal Cancer | rs7014346 | Intergenic | 8 |
| Ovarian/Prostate/ | rs6983267 | Intergenic | 8 |
| Colorectal Cancer | | | |
| Ovarian/Prostate/ | rs10505477 | Intergenic | 8q24 |
| Colorectal Cancer | | | |
| Ovarian/Prostate/ | rs10808556 | Intergenic | 8q24 |
| Colorectal Cancer | | | |
| Breast Cancer | rs2298083 | missense | 1 |
| Breast Cancer | rs2291533 | Intergenic | 3 |
| Breast Cancer | rs315675 | missense | 4 |
| Breast Cancer | rs4986790 | missense | 9 |
| Breast Cancer | rs8176740 | missense | 9 |
| Breast Cancer/ | rs1935 | missense | 10 |
| Ankylosing Spodylitis | | | |
| Breast Cancer | rs12422149 | missense | 11 |
| Breast Cancer | rs7313899 | missense | 12 |
| Breast Cancer | rs2879097 | missense | 17 |
| Breast Cancer/ | rs35018800 | missense | 19 |
| Autoimmune Disorders | | | |
| Breast Cancer | rs10415312 | missense | 19 |
| Breast Cancer | rs2822558 | Intergenic | 21 |
| Breast Cancer | rs9616915 | missense | 22 |
| Breast Cancer | rs3803662 | cds-synon | 16 |
| Breast Cancer | rs889312 | Intergenic | 5 |
| Breast Cancer | rs13387042 | Intergenic | 2 |
| Breast Cancer | rs1053485 | Intergenic | 10 |
| Breast Cancer | rs2981582 | Inntronic | 10 |
| Prostate Cancer | rs4430796 | Intronic | 17q12 |
| Prostate Cancer | rs7501939 | Intronic | 17q12 |
| Prostate Cancer | rs3760511 | nearGene-3 | 17q12 |
| Prostate Cancer | rs1859962 | Intergenic | 17q24.3 |
| Prostate Cancer | rs983085 | Intergenic | 17q24.3 |
| Schizophrenia | rS8029320 | Intergenic | 15 |
| Schizophrenia | rs1897786 | Intronic | 15 |
| Schizophrenia | rs999842 | Intronic | 15 |
| Schizophrenia | rs8038654 | Intronic | 15 |
| Schizophrenia | rs10438342 | Intronic | 15 |
| Schizophrenia | rs12141187 | Intergenic | 1 |
| Schizophrenia | rs6684174 | Intergenic | 1 |
| Schizophrenia | rs2644577 | Intergenic | 1 |
| Schizophrenia | rs4950437 | Intergenic | 1 |
| Schizophrenia | rs952477 | Intergenic | 1 |
| Schizophrenia | rs10793705 | Intronic | 1 |
| Schizophrenia | rs4132958 | Intergenic | 1 |
| Type 2 Diabetes | rs10282940 | UTR-3 | 8 |
| Type 2 Diabetes | rs10490072 | Intergenic | 2 |
| Type 2 Diabetes | rs10923931 | Intronic | 1 |
| Type 2 Diabetes | rs1153188 | Intergenic | 12 |
| Type 2 Diabetes | rs12304921 | Intronic | 12q13 |
| Type 2 Diabetes | rs13071168 | Intergenic | 3 |
| Type 2 Diabetes | rs17036101 | Intergenic | 3 |
| Type 2 Diabetes | rs17705177 | Intergenic | 17 |
| Type 2 Diabetes | rs1801282 | Intronic | 3 |
| Type 2 Diabetes | rs2641348 | missense | 1 |
| Type 2 Diabetes | rs2903265 | Intronic | 15q25 |
| Type 2 Diabetes | rs358806 | Intergenic | 3p14 |
| Type 2 Diabetes | rs4402960 | Intronic | 3 |
| Type 2 Diabetes | rs4506565 | Intronic | 10q25 |
| Type 2 Diabetes | rs4580722 | nearGene-3 | 4 |
| Type 2 Diabetes | rs4607103 | Intronic | 3 |
| Type 2 Diabetes | rs4655595 | Intronic | 1p31 |
| Type 2 Diabetes | rs5015480 | Intergenic | 10 |
| Type 2 Diabetes | rs5215 | missense | 11 |
| Type 2 Diabetes | rs5219 | missense | 11 |
| Type 2 Diabetes | rs6931514 | Intronic | 6 |
| Type 2 Diabetes | rs7020996 | Intergenic | 9 |
| Type 2 Diabetes | rs7578597 | missense | 2 |
| Type 2 Diabetes | rs7659604 | Intergenic | 4q27 |
| Type 2 Diabetes | rs7903146 | Intronic | 10q25 |
| Type 2 Diabetes/ | rs8050136 | Intronic | 16 |
| Obesity | | | |
| Type 2 Diabetes | rs864745 | Intronic | 7 |
| Type 2 Diabetes | rs9465871 | Intronic | 6p22 |
| Type 2 Diabetes | rs9472138 | Intergenic | 6 |
| Type 2 Diabetes/ | rs9939609 | Intronic | 16q12 |
| Obesity | | | |
| Obesity | rs12970134 | Intergenic | 18 |
| Obesity | rs17782313 | Intergenic | 18 |
| Obesity/Type 2 | rs9939609 | Intronic | 16q12 |
| Diabetes | | | |
| Obesity | rs1121980 | Intronic | 16 |
| Obesity | rs1558902 | Intronic | 16 |
| Obesity | rs17817449 | Intronic | 16 |
| Obesity | rs3751812 | Intronic | 16 |
| Obesity | rs9930506 | Intronic | 16 |
| Obesity/Type 2 | rs8050136 | Intronic | 16 |
| Diabetes | | | |
| Crohn's Disease | rs10210302 | nearGene-5 | 2q37 |
| Crohn's Disease | rs10761659 | Intergenic | 10q21 |
| Crohn's Disease | rs10883365 | Intergenic | 10q24 |
| Crohn's Disease | rs11209026 | missense | 1p31 |
| Crohn's Disease | rs805303 | Intronic | 1p31 |
| Crohn's Disease | rs17221417 | Intronic | 16q12 |
| Crohn's Disease | rs17234657 | Intergenic | 5p13 |
| Crohn's Disease | rs2066844 | missense | 16q12 |
| Crohn's Disease | rs12037606 | Intergenic | 1q24 |
| Crohn's Disease | rs6596075 | Intergenic | 5q23 |
| Crohn's Disease | rs6601764 | Intergenic | 10p15 |
| Crohn's Disease | rs6908425 | Intronic | 6p22 |
| Crohn's Disease | rs7807268 | Intergenic | 7q36 |
| Crohn's Disease | rs8111071 | Intronic | 19q13 |
| Crohn's Disease | rs9469220 | Intergenic | 6p21 |
| Crohn's Disease/ | rs2542151 | Intergenic | 18p11 |
| Type 1 Diabetes | | | |
| Crohn's Disease | rs4353135 | nearGene-3 | 1 |
| Crohn's Disease | rs4266924 | nearGene-3 | 1 |
| Crohn's Disease | rs55646866 | Intergenic | 1 |
| Crohn's Disease | rs6672995 | Intergenic | 1 |
| Crohn's Disease | rs107635144 | Intergenic | 1 |
| Crohn's Disease | rs10733113 | Intergenic | 1 |
| Ulcerative colitis | rs3737240 | Missense | 1 |
| Ulcerative colitis | rs13294 | Missense | 1 |
| Ulcerative colitis | rs3197999 | Missense | 3 |
| Ulcerative colitis | rs9268480 | cds-synon | 6 |
| Ulcerative colitis | rs660895 | Integenic | 6 |
| Bipolar disorder | rs420259 | Intronic | 16p12 |
| Bipolar disorder | rs10982256 | Intronic | 9q32 |
| Bipolar disorder | rs11622475 | Intronic | 14q32 |
| Bipolar disorder | rs1375144 | Intronic | 2q14 |
| Bipolar disorder | rs2609653 | Intergenic | 8p12 |
| Bipolar disorder | rs2953145 | Intronic | 2q37 |
| Bipolar disorder | rs3761218 | nearGene-5 | 20p13 |
| Bipolar disorder | rs6458307 | Intergenic | 6p21 |
| Bipolar disorder | rs683395 | Intronic | 3q27 |
| Bipolar disorder | rs7570682 | Intergenic | 2q12 |
| Coronary Artery | rs1333049 | Intergenic | 9p21 |
| Diseases | | | |
| Coronary Artery | rs4420638 | nearGene-3 | 19 |
| Diseases/Alzheimer's | | | |
| Coronary Artery | rs17672135 | Intronic | 1q43 |
| Diseases | | | |
| Coronary Artery | rs383830 | Intergenic | 5q21 |
| Diseases | | | |
| Coronary Artery | rs7250581 | Intergenic | 19q12 |
| Diseases | | | |
| Coronary Artery | rs10757274 | Intergenic | 9p21 |
| Diseases | | | |
| Coronary Artery | rs2383206 | Intergenic | 9p21 |
| Diseases | | | |
| Coronary Artery | rs10757278-G SNP | Intergenic | 9p21 |
| Diseases | is associated | | |
| | with | | |
| Coronary Artery | rs2383207 | Intergenic | 9p21 |
| Diseases | | | |
| Hypertension | rs11110912 | Intronic | 12q23 |
| Hypertension | rs1937506 | Intergenic | 13q21 |
| Hypertension | rs2398162 | Intronic | 15q26 |
| Hypertension | rs2820037 | Intergenic | 1q43 |
| Hypertension | rs6997709 | Intergenic | 8q24 |
| Hypertension | rs7961152 | Intronic | 12p12 |
| Rheumatoid Arthritis | rs11761231 | Intergenic | 7q32 |
| Rheumatoid Arthritis | rs615672 | Intergenic | 6 |
| Rheumatoid Arthritis | rs6457617 | Intergenic | 6 |
| Rheumatoid Arthritis | rs11162922 | Intergenic | 1p31 |
| Rheumatoid Arthritis | rs2837960 | Intergenic | 21q22 |
| Rheumatoid Arthritis | rs3816587 | Intronic | 4p15 |
| Rheumatoid Arthritis | rs6684865 | Intronic | 1p36 |
| Rheumatoid Arthritis | rs6920220 | Intergenic | 6q23 |
| Rheumatoid Arthritis | rs743777 | Intergenic | 22q13 |
| Rheumatoid Arthritis | rs9550642 | Intronic | 13q12 |
| Rheumatoid Arthritis/ | rs2104286 | Intronic | 10p15 |
| Type 1 Diabetes | | | |
| Rheumatoid Arthritis/ | rs2476601 | missense | 1 |
| Type 1 Diabetes | | | |
| Rheumatoid Arthritis/ | rs6679677 | Intergenic | 1p13 |
| Type 1 Diabetes | | | |
| Type 1 Diabetes | rs11171739 | Intergenic | 12q13 |
| Type 1 Diabetes | rs12708716 | Intronic | 16p13 |
| Type 1 Diabetes | rs1990760 | missense | 2 |
| Type 1 Diabetes | rs3087243 | nearGene-3 | 2 |
| Type 1 Diabetes | rs3764021 | cds-synon | 12p13 |
| Type 1 Diabetes | rs3788964 | Intronic | 2 |
| Type 1 Diabetes | rs6534347 | Intronic | 4q27 |
| Type 1 Diabetes | rs9270986 | Intergenic | 6 |
| Type 1 Diabetes | rs9272346* | nearGene-5 | 6 |
| Type 1 Diabetes | rs11052552 | Intergenic | 12p13 |
| Type 1 Diabetes | rs17166496 | Intronic | 5q31 |
| Type 1 Diabetes | rs17388568 | Intronic | 4q27 |
| Type 1 Diabetes | rs2544677 | Intergenic | 5q14 |
| Type 1 Diabetes | rs2639703 | Intronic | 1q42 |
| Type 1 Diabetes/CD | rs2542151 | Intergenic | 18p11 |
| Type 1 Diabetes/ | rs2104286 | Intronic | 10p15 |
| Rheumatoid Arthritis | | | |
| Type 1Diabetes/ | rs2476601 | missense | 1 |
| Rheumatoid Arthritis | | | |
| Type 1Diabetes/ | rs6679677 | Intergenic | 1p13 |
| Rheumatoid Arthritis | | | |
| Systemic Lupus | rs10798269 | Intergenic | 1 |
| Erythematosus | | | |
| Systemic Lupus | rs1143678 | missense | 16 |
| Erythematosus | | | |
| Systemic Lupus | rs12537284 | Intergenic | 7 |
| Erythematosus | | | |
| Systemic Lupus | rs3131379 | Intronic | 6 |
| Erythematosus | | | |
| Systemic Lupus | rs4548893 | nearGene-3 | 16 |
| Erythematosus | | | |
| Systemic Lupus | rs4963128 | Intronic | 11 |
| Erythematosus | | | |
| Systemic Lupus | rs729302 | Intergenic | 7 |
| Erythematosus | | | |
| Systemic Lupus | rs9888739 | Intronic | 16 |
| Erythematosus | | | |
| Systemic Lupus | rs1143679 | missense | 16 |
| Erythematosus | | | |
| Systemic Lupus | rs10516487 | missense | 4 |
| Erythematosus | | | |
| Systemic Lupus | rs17266594 | Intronic | 4 |
| Erythematosus | | | |
| Systemic Lupus | rs 11574637 | Intronic | 16 |
| Erythematosus | | | |
| Systemic Lupus | rs2070197 | UTR-3 | 7 |
| Erythematosus | | | |
| Systemic Lupus | rs2004640 | Intronic | 7 |
| Erythematosus | | | |
| Vitiligo | rs11078575 | Intronic | 17p13.2 |
| Vitiligo | rs12150220 | missense | 17p13.2 |
| Vitiligo | rs1877658 | Intronic | 17p13.2 |
| Vitiligo | rs2716914 | Intergenic | 17p13.2 |
| Vitiligo | rs2733359 | Intergenic | 17p13.2 |
| Vitiligo | rs35658367 | Intergenic | 17p13.2 |
| Vitiligo | rs3926687 | Intergenic | 17p13.2 |
| Vitiligo | rs4790796 | Intergenic | 17p13.2 |
| Vitiligo | rs4790797 | Intergenic | 17p13.2 |
| Vitiligo | rs6502867 | Intronic | 17p13.2 |
| Vitiligo | rs7223628 | Intergenic | 17p13.2 |
| Vitiligo | rs8182352 | Intergenic | 17p13.2 |
| Vitiligo | rs8182354 | Intergenic | 17p13.2 |
| Vitiligo | rs878329 | Intergenic | 17p13.2 |
| Vitiligo | rs925597 | nearGene-3 | 17p13.2 |
| Vitiligo | rs961826 | Intronic | 17p13.2 |
| Vitiligo | rs2670660 | Intergenic | 17p13.2 |
| Autoimmune thyroid | rs2072751 | missense | 1 |
| disease | | | |
| Autoimmune thyroid | rs671108 | missense | 1 |
| disease | | | |
| Autoimmune thyroid | rs6427384 | missense | 1 |
| disease | | | |
| Autoimmune thyroid | rs6679793 | missense | 1 |
| disease | | | |
| Autoimmune thyroid | rs35285785 | missense | 2 |
| disease | | | |
| Autoimmune thyroid | rs10186922 | Intergenic | 2 |
| disease | | | |
| Autoimmune thyroid | rs7578199 | missense | 2 |
| disease | | | |
| Autoimmune thyroid | rs7302981 | missense | 12 |
| disease | | | |
| Autoimmune thyroid | rs7975069 | missense | 12 |
| disease | | | |
| Autoimmune thyroid | rs2391191 | missense | 13 |
| disease | | | |
| Autoimmune thyroid | rs3783941 | missense | 14 |
| disease | | | |
| Autoimmune thyroid | rs2279961 | missense | 17 |
| disease | | | |
| Autoimmune thyroid | rs2856966 | missense | 18 |
| disease | | | |
| Autoimmune thyroid | rs7250822 | missense | 19 |
| disease | | | |
| Multiple sclerosis | rs3748816 | missense | 1 |
| Multiple sclerosis | rs6542517 | Intronic | 2 |
| Multiple sclerosis | rs6897932 | missense | 5 |
| Multiple sclerosis | rs6957669 | Intergenic | 7 |
| Multiple sclerosis | ATM-333 | missense | 11 |
| Multiple sclerosis | rs1918496 | missense | 12 |
| Multiple sclerosis | rs9897794 | missense | 17 |
| Multiple sclerosis | rs2229358 | cds-synon | 17 |
| Multiple | rs11554159 | missense | 19 |
| sclerosis/Ankylosing | | | |
| Spondylitis | | | |
| Multiple sclerosis | rs1800437 | missense | 19 |
| Ankylosing spondylitis | rs2272920 | missense | 1 |
| Ankylosing spondylitis | rs12143301 | missense | 1 |
| Ankylosing spondylitis | rs2296160 | missense | 1 |
| Ankylosing spondylitis | rs8192556 | missense | 2 |
| Ankylosing spondylitis | rs3197999 | missense | 3 |
| Ankylosing spondylitis | rs27044 | missense | 5 |
| Ankylosing spondylitis | rs17482078 | missense | 5 |
| Ankylosing spondylitis | rs10050860 | missense | 5 |
| Ankylosing spondylitis | rs30187 | missense | 5 |
| Ankylosing spondylitis | rs2303138 | missense | 5 |
| Ankylosing spondylitis | rs1456908 | missense | 7 |
| Ankylosing | rs1935 | missense | 10 |
| spondylitis/Breast Cancer | | | |
| Ankylosing spondylitis | rs2302250 | Intronic | 12 |
| Ankylosing spondylitis | rs3741927 | missense | 12 |
| Ankylosing spondylitis | rs7302230 | missense | 12 |
| Ankylosing spondylitis | rs1050931 | UTR-3 | 15 |
| Ankylosing spondylitis | rs9939768 | nearGene-3 | 16 |
| Ankylosing spondylitis/ | rs11554159 | missense | 19 |
| Multiple sclerosis | | | |
| Ankylosing spondylitis | rs709012 | missense | 20 |
| Autoimmune Disorders | rs12085435 | missense | 1 |
| Autoimmune Disorders | rs12067507 | missense | 1 |
| Autoimmune Disorders | rs1729674 | Intronic | 2 |
| Autoimmune Disorders | rs2232337 | missense | 3 |
| Autoimmune Disorders | rs1132200 | missense | 3 |
| Autoimmune Disorders | rs11171 | nearGene-5 | 7 |
| Autoimmune Disorders | rs697636 | missense | 12 |
| Autoimmune Disorders | rs34536443 | missense | 19 |
| Autoimmune Disorders/ | rs35018800 | missense | 19 |
| Breast Cancer | | | |
| Autoimmune Disorders | rs2303759 | missense | 19 |
| Autoimmune Disorders | rs1127291 | missense | 11 |

1.8 Materials and Methods

Disease Associated SNP Meta-Analysis and Mapping of Genomic Coordinates

Primary data sets of SNPs for meta-analysis of genomic coordinates of SNP variations identified in genome-wide association studies (GWAS) of up to 712,253 samples comprising 221,158 disease cases, 322,862 controls, and 168,233 case/control subjects of obesity GWAS were obtained from the following previously published studies:
Wellcome Trust Case Control Consortium. Genome-wide association study of 14,000 cases of seven common diseases and 3,000 shared controls. Nature 2007 447: 661-678.
Tenesa A, Farrington S M, Prendergast J G, et al. Genome-wide association scan identifies a colorectal cancer susceptibility locus on 11q23 and replicates risk loci at 8q24 and 18q21. Nat Genet 2008 40: 631-7.
Haiman C A et al., A common genetic risk factor for colorectal and prostate cancer. Nat Genet 2007 39: 954-6.
Zeggini E et al., Meta-analysis of genome-wide association data and large-scale replication identifies additional susceptibility loci for type 2 diabetes. Nat Genet 2008 40: 638-645.
Barton A. et al., Re-evaluation of putative rheumatoid arthritis susceptibility genes in the post-genome wide association study era and hypothesis of a key pathway underlying susceptibility. Hum Mol Genet. 2008 Apr. 22.
Remmers E F et al., STAT4 and the risk of rheumatoid arthritis and systemic lupus erythematosus. N Engl J. Med. 2007 357: 977-986.
Plenge R M et al., Two independent alleles at 6q23 associated with risk of rheumatoid arthritis. Nat Genet 2007 39: 1477-1482.
Thomson W et al., Wellcome Trust Case Control Consortium, Wilson A G, Marinou I, Morgan A, Emery P et al., Rheumatoid arthritis association at 6q23. Nat Genet. 2007 39: 1431-1433.
Wellcome Trust Case Control Consortium; Australo-Anglo-American Spondylitis Consortium (TASC), Burton PR et al., Association scan of 21 14,500 nonsynonymous SNPs in four diseases identifies autoimmunity variants. Nat Genet 2007 39: 1329-1337.
International Consortium for Systemic Lupus Erythematosus Genetics (SLEGEN), Harley J B et al., Genome-wide association scan in women with systemic lupus erythematosus identifies susceptibility variants in ITGAM, PXK, KIAA1542 and other loci. Nat Genet 2008 40: 204-210.
Nath S K et al., A nonsynonymous functional variant in integrin-alpha(M) (encoded by ITGAM) is associated with systemic lupus erythematosus. Nat Genet 2008 40: 152-154.
Kozyrev S V et al., Functional variants in the B-cell gene BANK1 are associated with systemic lupus erythematosus. Nat Genet 2008 40:211-216.
Hom G, et al., Association of systemic lupus erythematosus with C8orfl3-BLK and ITGAM-ITGAX. N Engl J. Med. 2008 358: 900-909.
Zheng S L, et al., Cumulative association of five genetic variants with prostate cancer. N Engl J Med 2008 358: 910-919.
Gudmundsson J, et al., Common sequence variants on 2p15 and Xp11.22 confer susceptibility to prostate cancer. Nat Genet 2008 40: 281-283.
Jin Y, et al., NALP1 in vitiligo-associated multiple autoimmune disease. N Engl J Med 2007 356:1216-1225.
Fisher S A, et al., Genetic determinants of ulcerative colitis include the ECM1 locus and five loci implicated in Crohn's disease. Nat Genet 2008 40:710-712.
Cox A, et al., A common coding variant in CASP8 is associated with breast cancer risk. Nat Genet 2007; 39:352-8.
Easton D F, et al., Genome-wide association study identifies novel breast cancer susceptibility loci. Nature 2007; 447:1087-93.
Hunter D J, et al., A genome-wide association study identifies alleles in FGFR2 associated with risk of sporadic postmenopausal breast cancer. Nat Genet 2007; 39:870-4.
Stacey S N et al., Common variants on chromosomes 2q35 and 16q12 confer susceptibility to estrogen receptor-positive breast cancer. Nat Genet 2007; 39:865-9.
Tomlinson I P et al., A genome-wide association study identifies colorectal cancer susceptibility loci on chromosomes 10p14 and 8q23.3. Nat Genet 2008 40: 623-30.
Jaeger E et al., Common genetic variants at the CRAC1 (HMPS) locus on chromosome 15q13.3 influence colorectal cancer risk. Nat Genet. 2008 40: 26-8.
Broderick P, et al., A genome-wide association study shows that common alleles of SMAD7 influence colorectal cancer risk. Nat Genet 2007 39: 1315-7.
Tomlinson I, et al., A genome-wide association scan of tag SNPs identifies a susceptibility variant for colorectal cancer at 8q24.21. Nat Genet. 2007 39: 984-8.
Gruber S B, et al., Genetic Variation in 8q24 Associated with Risk of Colorectal Cancer. Cancer Biol Ther. 2007 6

Mapping of the SNP genomic coordinates was performed based on the NCBI release of Human Genome Build 36.3 (reference assembly). Genomic coordinates of the human K4-K36 domains and human lincRNAs are publically available in the online Supplemental data set of Khalil A M et al., Many human large intergenic noncoding RNAs associate with chromatin-modifying complexes and affect gene expression. Proc Natl Acad Sci U S A. 2009 Jul. 1.

Genomic coordinates and gene names of the human bivalent domain genes were obtained from the recently published study, Ku, M. et al., Genomewide analysis of PRC1 and PRC2 occupancy identifies two classes of bivalent domains. PLoS Genet. 2008; 4: e1000242.

Cell Lines

Human BJ1, U937, and THP-1 cell lines were obtained from ATCC. hTERT-immortalized BJ1 cells were previously described in Holt SE et al., Resistance to apoptosis in human cells conferred by telomerase function and telomere stability. Mol Carcinog. 1999; 25: 241-8.

Microarray Gene Expression Analysis

Sense and anti-sense variants of the 52 nt rs2670660 sequence were chemically synthesized, cloned into GFP-expressing lentiviral vectors, and transfected into BJ1 cells. Corresponding BJ1 cell line variants were isolated by sterile FACS sorting to contain >90% of GFP-expressing cells, expanded in vitro in monolayer cultures, and analyzed for gene expression.

Technical and analytical aspects as well as stringent QC and statistical protocols for gene expression analysis experiments is essentially as described in the following published works:
Glinsky, G V et al., Microarray analysis identifies a death-from-cancer signature predicting therapy failure in patients with multiple types of cancer. J Clin Invest; 2005; 115: 1503-1521.
Glinsky G V et al., Classification of human breast cancer using gene expression profiling as a component of the survival predictor algorithm. Clin Cancer Res. 2004 10: 2272-2283.
Glinsky G V et al., Gene expression profiling predicts clinical outcome of prostate cancer. J Clin Invest. 2004 113: 913-923.
Glinsky G V, et al., Microarray analysis of xenograft-derived cancer cell lines representing multiple experimental models of human prostate cancer. Mol Carcinog. 2003 37: 209-221.

Briefly, the array hybridization and processing, data retrieval and analysis were carried out using standard sets of the Affymetrix equipment, software, and protocols in a state-of-the-art Affymetrix microarray core facility. RNA was extracted from cell cultures of two independent biological replicates for each experimental condition and analyzed for sample purity and integrity using a BioAnalyzer (Agilent). Expression analysis of 54,675 transcripts was carried out for each sample in duplicate using Affymetrix HG-U133A Plus 2.0 arrays. Data retrieval and analysis was performed using MAS5.0 software and concordant changes of gene expression for each experimental condition were determined at the statistical threshold p value <0.05 (two-tailed T-test).

mIcroRNA Isolation and Activity Analysis.

miRNA was extracted from adherent cells lysed on culture plates using the miNana miRNA Isolation kit (Ambion). Homogenized cell lysates were frozen at −80° C. for at least 24 hours prior to miRNA purification. miRNA concentration was checked using a NanoDrop (Thermo Scientific) before checking quality on a Bioanalyzer (Agilent Technologies).

To assay the activity of microRNAs in transfected cells we used a miRNA Luciferase Reporter Vector (Signosis) specific for the microRNA of interest. The target site sequence of the reporter vector is complementary to the miRNA, therefore a decrease in luciferase signal would indicate an increase in microRNA activity. Cells were transfected with the reporter vector using FuGENE 6 Transfection Reagent (Roche); the transfection was allowed to run 48 hours before the cells were lysed using Luciferase Cell Culture Lysis Reagent (Promega). The lysates were read using the FLUOstar OPTIMA system (BMG Lab Technologies), with 20 micro liters of Luciferase Assay Reagent (Promega) injected into each well immediately prior to reading.

miRNA Expression Analysis

To analyze a spectrum of miRNA activity in the infected cell lines, we performed qPCR using the TaqMan Human MicroRNA Array v1.0 (Applied Biosystems) run on the 7900HT Fast Real-Time PCR System, fitted with the specific block to run 384-well TaqMan Low Density Arrays (Applied Biosystems). This TaqMan array is distributed on a micro fluidics card, which allows for high reproducibility with minimal error. The array contains 365 different human miRNA assays and two small nucleolar RNAs that function as endogenous controls for data normalization. All miRNA samples were analyzed for quality control and processed at the Functional Genomics Core of the University of Rochester in Rochester, N.Y. We used the SDS 2.2 software, the platform for the computer interface with the 7900HT PCR System, to generate normalized data, compare samples, and calculate RQ.

Cell Staining and Flow Cytometry

Cells were stained at a concentration of 1×10⁶ cells per 100 microliters (ul) of HEPES buffered saline (HBSS) with 2% HICS. Antibodies at appropriate dilutions (CD14-Pacific Blue, Biolegend, Inc; and CD11b-Alexa Fluor® 647, Biolegend, Inc) were added. Staining duration was for 30 min with rotation at 4° C. Cells were then washed with staining medium three times and resuspended in staining medium. The stained specimens were then analyzed using FACSVantage (BD Biosciences, San Diego, Calif.; http://www.bdbiosciences.com) or FACSAria with either Diva or CellQuest software (BD Biosciences): The cell counter of the flow cytometers was used to determine cell numbers. Cells were collected into HBSS with 2% HICS.

Induced Differentiation of 0937 and THP-1 Cells

Approximately 2×10⁶ U937 or THP-1 cells (5×10⁵ cells/ml) in a 25 cm flask were induced to differentiate by treatment with 20 uM PMA (Sigma-Aldrich) for 4 days.

Lentivirus Production and Generation of Stably Transfected BJ1, 0937, and THP-1 Cells

Allele-specific sense and anti-sense variants of the 52 nucleotide rs2670660 sequence, SEQ ID NO: ______ (5′ CACAA GTGAT CTACC AGTCT TTTAA A(G/A)TTC TATTA TTAAA ACCCA AACAT GC 3′) were chemically synthesized and cloned sequentially into pUC57 plasmid by Ec0RV (GeneScript Corporation) and pCDH-CMV-MCS-EF1-copGFP plasmid by EcoR1 and Not1 (SystemsBio). The integrity and molecular identity of the synthetic sequences as well as designed plasmid vectors were monitored by restriction enzyme mapping analysis and direct sequencing. Lentiviruses were generated by co-transfecting pLentiviral vector with GFP only plasmids (control cultures) or GFP plasmids with synthetic, allele-specific 52 nt sequences of the SNP rs2670660 and packaging mix (Invitrogen) into 293FT cells using Lipofectamine 2000 according to the manufacturer's instructions (Invitrogen), and then BJ1, U937., or THP-1 cells were infected with viral supernatant for 24 hr. Flow cytometry analysis for GFP expression were performed to confirm the infection and assess the transfection efficiency. Experiments were carried out using cultures with transfection efficiency >90%.

Colony Growth Assay

Sense and anti-sense variants of the 52 nt snpRNA were synthesized, cloned into GFP-lentiviral vectors, and transfected into BJ1 cells. GFP-expressing cells were isolated by flow cytometry and enriched populations (>90% GFP positive) were used for assays. Cells from sub-confluent cultures (about 70% confluence) were seeded in triplicates into Ewell plates (100 cells per well), cultured for 2 weeks, and then stained with 0.1% crystal violet for 5 min. Plates were scanned and number of colonies containing >50 cells was counted.

Protocols for Identification of Endogenous Trans-Regulatory Small RNAs Encoded by the SNP rs2670660

1. Extract small RNA from cells (mirVana™ miRNA Isolation Kit from Ambion, Inc., according to manufacturer's directions)
2. Detect if there is DNA contamination by performing PCR using extracted RNA as template and beta-actin as primer
3. Synthesize cDNA from small RNA using standard protocols
4. Perform first PCR using primer set 2 (GC2F and GC2R): In a clean tube on ice, combine PCR reagents to a 25 ul final volume: Water, RNase-free; PCR Buffer (10×) 2.5 ul; PCR Nucleotide Mix (10 mM) 0.5 ul; Taq DNA polymerase (50×) 0.5 ul; template; Forward primer (10 uM) 1 ul (0.4 uM final conc.); Reverse primer (10 uM) 1 μl(0.4 uM final conc.). Thermal cycle profile: 95° C. 3 min followed by 40 or more cycles: 95° C. 30s, 55° C. 30s, 72° C. 1 min (or 1-2 min per kilobase); followed by final extension 72° C. 3 min and hold at 4° C.
5. Clean up PCR product and evaluate cleanup PCR product on 1.2% gel (Montage PCR Centrifugal Filter Devices available from Millipore, Inc., according to manufacturer's instructions)
6. Perform nested PCR using cleanup of the first PCR product as template and primer set 1 (GC1F and GC1R) and evaluate nested PCR product on 1.2% gel (protocol as per no. 4, supra)
7. Cut the DNA band of interest from the gel, extract and purify the DNA for further sequencing analysis (QIAquick Gel Extraction Kit, Qiagen, Inc., according to manufacturer's instructions)

Statistical and Bioinformatics Analysis

Detailed protocols for data analysis and documentation of the sensitivity, reproducibility, and other aspects of the quantitative statistical microarray analysis using Affymetrix technology have been described in:
Stack J H et al., IL-converting enzyme/caspase-1 inhibitor VX-765 blocks the hypersensitive response to an inflammatory stimulus in monocytes fromfamilial cold autoinflammatory syndrome patients. J Immunol 2005; 175:2630-4.
Holt S E et al., Resistance to apoptosis in human cells conferred by telomerase function and telomere stability. Mol Carcinog. 1999; 25: 241-8.
Glinsky, G V et al., Microarray analysis identifies a death-from-cancer signature predicting therapy failure in patients with multiple types of cancer. J Clin Invest; 2005; 115: 1503-1521.
Glinsky G V et al., Classification of human breast cancer using gene expression profiling as a component of the survival predictor algorithm. Clin Cancer Res. 2004 10: 2272-2283.
Glinsky G V et al., Gene expression profiling predicts clinical outcome of prostate cancer. J Clin Invest. 2004 113: 913-923.
Glinsky G V, et al., Microarray analysis of xenograft-derived cancer cell lines representing multiple experimental models of human prostate cancer. Mol Carcinog. 2003 37: 209-221.

Briefly, forty to sixty percent of the surveyed genes were called present by Affymetrix Microarray Suite version 5.0 software in these experiments. The concordance analysis of differential gene expression across the data sets was performed using Affymetrix MicroDB version 3.0 and DMT version 3.0 software as described in the references above. The microarray data was processed using the Affymetrix Microarray Suite version 5.0 software and statistical analysis of the expression data set was performed using the Affymetrix MicroDB and Affymetrix DMT software. The Pearson correlation coefficient for individual test samples and the appropriate reference standard were determined using GraphPad Prism version 4.00 software (GraphPad Software). The significance of the overlap between the lists of differentially-regulated genes was calculated by using the hypergeometric distribution test (See Seila, A. C. et al. Divergent transcription from active promoters, Science (2008) 322:1849-51).

Expression profiling data included 697 clinical samples obtained from 185 control subjects and 350 patients diagnosed with 9 common human disorders including Crohn's disease (59 patients), ulcerative colitis (26 patients), rheumatoid arthritis (20 patients), Huntington's disease (17 patients), autism (15 patients), Alzheimer's disease (36 patients), obesity (14 subjects), prostate cancer (64 patients), and breast cancers (99 patients). Microarray data and associated clinical information are publically available in the Gene Expression Omnibus (GEO) database maintained by the National Center for Biotechnology Information using the following GEO accession numbers: GDS2601; GDS810; GDS2824; GDS1615; GDS711; GDS1480; GDS2545; GDS1331; GDS1407; GDS3203; GDS2255. Genomic information related to the PluriNet network genes is publically available from the Stem Cell Mesa microarray data server and also from Stem Cell Matrix.

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. An isolated small non-coding RNA molecule transcribed from an intergenic region of the human genome, wherein the RNA molecule is less than 300 nucleotides and the intergenic region contains at least one small nucleotide polymorphism (SNP) associated with one or more human diseases or disorders.

2. The RNA molecule of claim 1, wherein the cDNA form of the RNA molecule comprises a sequence selected from the group consisting of SEQ ID NOs: 1-101, 332, and 333.

3. The RNA molecule of claim 1, wherein the SNP is selected from the group consisting of rs2670660, rs6596075, rs6983561, rs16901979, rs13281615, rs10505477, rs10808556, rs6983267, rs7014346, rs7000448, rs1447295, rs2820037, rs889312, rs1937506, rs13387042, rs7716600, rsl 249433, and rs3803662.

4. The RNA molecule of claim 3, wherein the cDNA form of the RNA molecule comprises a sequence selected from the group consisting of SEQ ID NOs: 1, 4, 6, 7, 9-18, 39, 88-90, 332, and 333.

5. The RNA molecule of claim 4, wherein the cDNA form of the RNA molecule comprises a sequence selected from the group consisting of SEQ ID NOs: 1, 7, 332, and 333.

6. A vector comprising the cDNA form of the RNA molecule of claim 1.

7. A cell comprising the vector of claim 6.

8. A kit comprising, in one or more containers, the vector of claim 6 and instructions for expressing the RNA molecule from the vector.

9. A kit comprising, in one or more containers, the cell of claim 6 and instructions for expressing the RNA molecule in the cell.

10. A kit comprising, in one or more containers, the vector of claim 6 and one or more polynucleotide primers for amplifying the eDNA molecule.

11. The kit of claim 10, wherein the one or more primers comprises a sequence selected from the group consisting of SEQ ID NOs: 102-331.

12. The kit of claim 11, wherein the one or more primers comprises a sequence selected from the group consisting of SEQ ID NOs: 102-161.

13. The kit of claim 10, wherein the one or more primers comprises a sequence selected from the group consisting of SEQ ID NOs: 102, 103, 114, 115, 326, and 327.

14. A method for detecting the small non-coding RNA molecule of any one of claim 1 in a sample from a subject, the method comprising the step of detecting the cDNA form of the small non-coding RNA molecule in the sample.

15. The method of claim 14, wherein the cDNA form is detected by a method comprising reverse transcription and polymerase chain reaction (RT-PCR) technology.

16. The method of claim 14, wherein the cDNA form is detected by a method comprising nucleic acid hybridization technology.

17. The method of claim 14, further comprising the steps of isolating the small RNA fraction from the sample and converting the RNA into cDNA prior to the step of detecting the cDNA in the sample.

18. The method of claim 14, wherein the method comprising detecting the cDNA form of the RNA molecule having a sequence selected from the group consisting of SEQ ID NOs: 1, 7, 332, and 313.

19. A method for evaluating the risk that a human subject will develop a disease or condition associated with a specific allele of an SNP (“the pathological allele”) by detecting the presence of an RNA molecule of claim 1 in a sample from the subject, wherein the RNA molecule is transcribed from the pathological allele, and wherein detection of said RNA molecule indicates that the subject has an increased risk for developing the disease or condition and the failure to detect said RNA molecule indicates that the subject has a decreased risk for developing the disease or condition.

20. The method of claim 19, further comprising detecting the expression level of the RNA molecule transcribed from the pathological allele relative to its expression in a population of healthy subjects, wherein an increased or decreased level of expression relative to the population of healthy subjects indicates that the subject has an increased risk for developing the disease or condition.

21. The method of claim 19, wherein the step of detecting the presence of an RNA molecule transcribed from the pathological allele is performed indirectly, by detecting the expression of one or more genes whose expression is regulated by the RNA molecule.

22. A method for diagnosing a disease or condition associated with a specific allele of an SNP (“the pathological allele”) in a human subject, the method comprising detecting the presence of an RNA molecule of claim 1 in a sample from the subject, wherein the RNA molecule is transcribed from the pathological allele, and wherein the disease or condition is positively diagnosed if the RNA molecule is detected in the sample.

23. A method for treating, preventing, or ameliorating a disease or condition associated with a specific allele of an SNP (“the pathological allele”) in a subject in need thereof, the method comprising administering one or more therapeutic agents that act to suppress the expression or antagonize the activity of an RNA molecule of claim 1, wherein the RNA molecule is transcribed from the pathological allele.

24. The method of claim 14, wherein the subject is human.

25. The method of claim 14, wherein the sample is a blood, tissue, or cell sample.

26. The method of claim 19 wherein the disease or condition is selected from the group consisting of Crohn's disease, rheumatoid arthritis, Huntington's disease, Alzheimer's disease, breast cancer, prostate cancer, autism, and obesity.

27. An apparatus for evaluating a disease or condition, or evaluating the risk of developing a disease or condition, in a subject, the apparatus comprising a model configured to evaluate a dataset for the subject to thereby evaluate the risk of disease in the subject, wherein the model is based upon determining the similarity in the expression profile of a defined set of genes in a sample from the subject and the expression profile for that set of genes in one or more reference sets of the model, wherein a reference set comprises one or more of a population of healthy subjects and a population of subjects suffering from the disease, wherein the set of genes is a set of genes whose expression is regulated by a small RNA molecule of claim 1.


SEQUENCE LISTING (333 sequences)

SEQ ID NO: 1 - DNA, 52 bp, Homo sapiens
  allele (1)..(52): rs2670660; wherein r is g or a
cacaagtgatctaccagtcttttaaarttctattattaaaacccaaacatgc

SEQ ID NO: 2 - DNA, 151 bp, Homo sapiens
  allele (1)..(151): rs6458307; wherein y is c or t
tctttaatacagattgggaagaggattactttttctgtctcaggttcttcaggataaaggataaagatttggagatcgtttaaaagcttttatataaatgctcattcaytgagttcaaatacttttaaaatgtcctggcagttgaaagtta

SEQ ID NO: 3 - DNA, 131 bp, Homo sapiens
  allele (1)..(131): rs9472138; wherein y is c or t
gaacacttctgttaccctaagcacgttctcctcataycgtttgtcgtcaatccctaccacggctaccagtctcaggcagctactaatctatctgctttttttctgtgtaattttgccttttccagaaagtc

SEQ ID NO: 4 - DNA, 111 bp, Homo sapiens
  allele (1)..(111): rs6596075; wherein s is g or c
atttgtgttcaagcctccttccatgggaagaaccagcggtggacctgaagagctctgccttcaaacagatgattcactcasaacaggttgctggtgactgaacctcagtga

SEQ ID NO: 5 - DNA, 150 bp, Homo sapiens
  allele (1)..(150): rs2544677; wherein s is g or c
taatctttgtctttatgaasgtctagaggattctaccataaaattaggaaagataagttagaaatgttgaaacatagaaagtattataactagaacgcatttaatacttgtatttttaatttttgagacagtcttcctctgtcacccagg

SEQ ID NO: 6 - DNA, 114 bp, Homo sapiens
  allele (1)..(114): rs6983561; wherein m is a or c
atagaacatatagcacmaaatgattatatcaatagaatgctaattgcatatcaaggatatttggtataatacaaattattctaccttaaacatatggaaatttgtggtccatga

SEQ ID NO: 7 - DNA, 135 bp, Homo sapiens
  allele (1)..(135): rs16901979; wherein m is a or c
agtgtggggtctttgttgtggagcagtgttaatgatttagcattacttatmtctggcaaatggtatttttgagataacatgttatggaagaaagtgaactgaacttggaagtttgaagatctcgattgaagtatc

SEQ ID NO: 8 - DNA, 141 bp, Homo sapiens
  allele (1)..(141): rs672888; wherein y is c or t
gggcattttctgtgctactattcttaagagaattatctcactcaatcctcactgcagctctaggagctagatactgttattgycactttcttaaaggtaaagaaacacagatattaggcctattgccagcatcactcagca

SEQ ID NO: 9 - DNA, 173 bp, Homo sapiens
  allele (1)..(173): rs13281615; wherein r is g or a
gacacgtggaatttactcttttgataaattggtaactatgaatctcatcaaaagaargcagaacgcagatattctgagtagggggtttgggggagaaataagagtgattcctcctatctgctgctagggccataaagacactacaccaagaggaagtgtaggcttggccaggt

SEQ ID NO: 10 - DNA, 118 bp, Homo sapiens
  allele (1)..(118): rs10505477; wherein y is c or t
ccgtgggaaacaaagtcttccactgggcttattctgtgtcatgtgtcaccacttgtctatcaaacaggaagccttaaytggagatgaagatttagaaaaggggcaaagtcagtattga

SEQ ID NO: 11 - DNA, 151 bp, Homo sapiens
  allele (1)..(151): rs10808556; wherein y is c or t
ctccatagagcctgcagagggcactagactgggaattagaaaacctgatttcccttccagctccayctctgaccaattgcctgaccctggtcaaattgcttaacctcttcctatctcagctccctatccataaaacagagggacgaataaa

SEQ ID NO: 12 - DNA, 154 bp, Homo sapiens
  allele (1)..(154): rs6983267; wherein k is g or t
tcctttgagctcagcagatgaaagkcactgagaaaagtacaaagaatttttatgtgctattgactttattttattttatgtgggggagggagccggccccagctggaaagctgctttctctgaatcaaagggcaggaacccagcaagtttctca

SEQ ID NO: 13 - DNA, 126 bp, Homo sapiens
  allele (1)..(126): rs7014346; wherein r is g or a
gcttgcagcttctgcctaatgttgacttacagttcaagatggcttctggagtgctaccrttacatccatgttgtaggctagaaggaaaagggcaatggcctgaagaggaagggagagttcctgtta

SEQ ID NO: 14 - DNA, 135 bp, Homo sapiens
  allele (1)..(135): rs7000448; wherein y is c or t
gagcagaggagcagyatttttgagaatctggccaatatggaaagatttgctgacatattcagatttgagactttttttttttttagacggagttttgctcttgccactcaggctggagtgcagtggcacaatctc

SEQ ID NO: 15 - DNA, 174 bp, Homo sapiens
  allele (1)..(174): rs1447295; wherein m is a or c
tgagttgcacgccagacactatactagatgatgggacaactaaagggtaatgaacagttctgtctctatgtaaaaataataatgatgatgatgatgagatgggacttcaattgaggaagtgccattggggaggtatgtaaaamgtgctatggaaaaaaagcaacaggaacccct

SEQ ID NO: 16 - DNA, 190 bp, Homo sapiens
  allele (1)..(190): rs2820037; wherein w is t or a
gtgattgctctaattgccaagtacagaaaaagttactgggtgtgttcatagatctagtagctctattgtgaggtgaattttagtcaggacttcaattatcacatagttttcttgagcctccawtctaaaagagagcctgtgattactcttttgttctttaggtattaacatcaacatagacctcatgcgc

SEQ ID NO: 17 - DNA, 75 bp, Homo sapiens
  allele (1)..(75): rs889312; wherein m is c or a
atgcccctgctggagaaaggmatgtgcaaattaagagactacaaatcagtttgaaaactcaacgactccttccca

SEQ ID NO: 18 - DNA, 156 bp, Homo sapiens
  allele (1)..(156): rs1937506; wherein r is g or a
cgggaaagtaaaaattgttatctcattcatattcaaaaatttgataaaatcaggcttggaaaatgtgatttattaggtgtcaaataatgaagttatacctgtggagartattagaagtggaacattgtaatggatatgtccaaaggattggtcctc

SEQ ID NO: 19 - DNA, 99 bp, Homo sapiens
  allele (1)..(99): rs4242382; wherein r is g or a
cccagggaacattttgtccctctagttatcttcccrcaggcccatcaagaatcaggcagtaggtgaaaaagaaacacagagaacctaggaacacaatag

SEQ ID NO: 20 - DNA, 184 bp, Homo sapiens
  allele (1)..(184): rs7017300; wherein m is c or a
gagccaggacatcagaaagaaaattaaaaacaaagtggaatacagtgtgaagattgatttggggcaaaagatttgaaactaagaccatgaacaatgagattcgttaatggagtttccctttgtatgatgcctagamccagcaacagggcagttgcagtgatttaaggatgactcacagggatgg

SEQ ID NO: 21 - DNA, 166 bp, Homo sapiens
  allele (1)..(166): rs10090154; wherein y is c or t
ttctctccagattgatacacagctttaatgcaattcttataaaaatctctgcaagatttttttgtaaayatagctaaaacaatattggaaaaaaaatagtgaagtggtattccaaggcttactatatggccagagtagtccagactgtggtattggcagaggcatt

SEQ ID NO: 22 - DNA, 139 bp, Homo sapiens
  allele (1)..(139): rs7837688; wherein k is g or t
ttcacaggaaaattgagcagaaagtacaaagagctcctgtatatcccctacccccacacattcacagcctccctcattaccaacatttcccactagagtggtgcatttkgtacaattgggtctatgttgacacgtcatt

SEQ ID NO: 23 - DNA, 72 bp, Homo sapiens
tgctcctgtctcccaaactctagatgccacgtgggcgctgtagccccacttcgccaatgccttggttcgggc

SEQ ID NO: 24 - DNA, 87 bp, Homo sapiens
  allele (1)..(87): rs2542151; wherein k is g or t
gggckcttcctgagactctcattttcctaatttcactaacttcacaccttcttgctaattctgattatttttcctctgcgataggga

SEQ ID NO: 25 - DNA, 158 bp, Homo sapiens
  allele (1)..(158): rs16892766; wherein m is c or a
acggtcagacgcaaacagtttcaagactattmgctgttaaaggttatgccttatgtcacccaaaagggttttcccctagatttatagcacaaactcatggaagatttattgccgtcttaattttttccccaattttaactttamgaacagtcagcctg

SEQ ID NO: 26 - DNA, 141 bp, Homo sapiens
  allele (1)..(141): rs6997709; wherein k is g or t
ttgaccaaattgaagaattggtttgttctcacctaagttctatcaagccaaataagtkatgggacaggatgaaaaagatttttcctgacgtgaaaggatttgggtagtcacccattgaatgttctcatggagatcaagtct

SEQ ID NO: 27 - DNA, 85 bp, Homo sapiens
  allele (1)..(85): rs6457617; wherein y is c or t
tagtcayatctgctcatggactcaacaaacagtaattgagtccactgactgcatttcggaaatccacactcatgatcttcctctg

SEQ ID NO: 28 - DNA, 100 bp, Homo sapiens
  allele (1)..(100): rs9469220; wherein r is g or a
ataaattaccattcaaactgccrgtagaaatataaaattgtaaggaataaattccacaaaaaaatacagtgttttaattacaaaaatttaccatgcagca

SEQ ID NO: 29 - DNA, 111 bp, Homo sapiens
  allele (1)..(111): rs9469220; wherein r is g or a
tggcagtccaagctactaagaagcacaaataaaatatatagtagcagggggagatgggaagggtgagagaatgtaggataaattaccattcaaactgccrgtagaaatata

SEQ ID NO: 30 - DNA, 177 bp, Homo sapiens
  allele (1)..(177): rs9469220; wherein r is g or a
tggcagtccaagctactaagaagcacaaataaaatatatagtagcagggggagatgggaagggtgagagaatgtaggataaattaccattcaaactgccrgtagaaatataaaattgtaaggaataaattccacaaaaaaatacagtgttttaattacaaaaatttaccatgcagca

SEQ ID NO: 31 - DNA, 126 bp, Homo sapiens
  allele (1)..(126): rs660895; wherein r is g or a
ctgtctgatgggagtgaagattcttccttcaggaatggaaggggatgcacagagtgaagccacccaacaaaaacaagacttgtatrgctatagatggaagggaaatcaaccaggaaattattttgg

SEQ ID NO: 32 - DNA, 81 bp, Homo sapiens
  allele (1)..(81): rs615672; wherein s is g or c
gtggttaggaaaasagaaataagaacaacagcagaatgcaccgtcaggtactttggaagtcacagaagggaaaagggcagg

SEQ ID NO: 33 - DNA, 123 bp, Homo sapiens
  allele (1)..(123): rs9270986; wherein m is c or a
atgttcatcagtggtcacaaatataatgtatctaaaatagggacagtaagaaattactgggcataactagmaggtgccatgggatgtgcctggaaagcttctcatgacgacctaccatgagcc

SEQ ID NO: 34 - DNA, 71 bp, Homo sapiens
  allele (1)..(71): rs9270986; wherein m is c or a
atgttcatcagtggtcacaaatataatgtatctaaaatagggacagtaagaaattactgggcataactagm

SEQ ID NO: 35 - DNA, 203 bp, Homo sapiens
  allele (1)..(203): rs10186922; wherein r is g or a
cagctctgactcccaactccacacccccatgtacttcttcctctccaacctgtgctgggctgacatcggtttcacctcgcccatggttcccaagatgatcrtggacatgcagtcgcatagcagagtcatctctcatgcgggctgcctgacacggatgtctttcttggtcctttttgcatgtatagaagacatgctcctgactg

SEQ ID NO: 36 - DNA, 167 bp, Homo sapiens
  allele (1)..(167): rs11159647; wherein r is g or a
tgctcactacctgggtgcaatatactcatatagcaaagctgcacatrtatctaacataacattgaaattttaaaaataggacattttaatacaaaattagatttaaaagtaattactattagcgaaaataagtcacaaccatttagaaatctgaaaaatgctgacaa

SEQ ID NO: 37 - DNA, 201 bp, Homo sapiens
  allele (1)..(201): rs2609653; wherein y is c or t
tgtgcacaagagcattgttttctagcatatacttattttaactatttttagaagcayttcgcattttgaaaagtgaaaataacctaagtgttcatcaatggatgaatggaaaaagaaactgtggtacgtatatacaatggaatattatacggctctaaaaaagaatgggatcctgccatctgtcacaacatggatgatcct

SEQ ID NO: 38 - DNA, 208 bp, Homo sapiens
  allele (1)..(208): rs7570682; wherein r is g or a
agtgatggagtggcataggtaatttctggaatgactgaagtaaatataatcagctcactttaaaatgaattttttcagtataaagtaactctctggaarttgacatgaagtttgatcagaaattaaggcagaaggtatgtgaaacagtagaaactgtagatatgagtataaaaaaagtgggtggcaagggataaggaagcatgtaggg

SEQ ID NO: 39 - DNA, 130 bp, Homo sapiens
  allele (1)..(130): rs13387042; wherein r is g or a
cagaaagaaggcaaatggargctacagaaaccaaggatttccttgttgaatcgaatcttccttcaatcttccttcaccacactagtggatctccctgtgggagggatgttgagagtgctccgtgtttttt

SEQ ID NO: 40 - DNA, 125 bp, Homo sapiens
  allele (1)..(125): rs2291533; wherein s is c or g
ttttttaatttatacttcctcatggttctcttggatatcctctggaactgtttagaagactgaagaatttcatcccccagaaactcacastgttgaagctcagcatgtctttgggccagtagctt

SEQ ID NO: 41 - DNA, 158 bp, Homo sapiens
  allele (1)..(158): rs2822558; wherein r is g or a
ttctcgacaaaagttttccactggggaaattattaacttgatgtcagcaactcatggacttgacarcaaacctcaatctcctctggtctgccccttttcaaatcctaatggccgtatatctcctttggcaagagctgggtccagcagtgttagcaggg

SEQ ID NO: 42 - DNA, 181 bp, Homo sapiens
  allele (1)..(181): rs10795668; wherein r is g or a
ttgttttcaggagttttcatctatgagcagcagcagaaagagaaaaagttagattcttarattccatgattttatatttcccaccaaggtacaagtatttctacttttctacctgattgtctctactttcctccatgtgtatttcttttcttttcttttctttttcagacggagtctcgct

SEQ ID NO: 43 - DNA, 182 bp, Homo sapiens
  allele (1)..(182): rs4779584; wherein y is c or t
agctgctataagatgggctgagttagaaaaacctaacagcccatcctaatagactgaatgttctattgtttgatgaatgttatgtgccagtagaacttgttgataagccattcttcygaacagaaaccataactatayacacaggaaacaaaaatatttgtaatggcttttagcagtggcaa

SEQ ID NO: 44 - DNA, 210 bp, Homo sapiens
  allele (1)..(210): rs10757274; wherein r is g or a
agcttctcccccgtgggtcaaatctaagctgagtgttgrgacataattgaaattcactagatagataggagataggggtagggaattctaatcagagggaatagcacatgtaaggcaaacaatacagtgcatctgggaaagctatacaattttattgttataggacaaatgttggggaatgttgagagatggaactggagagtgaggcag

SEQ ID NO: 45 - DNA, 133 bp, Homo sapiens
  allele (1)..(133): rs10757278; wherein r is g or a
gttaagttagttggaactgaactgaggccagacagggctgtgggacaagtcagggtgtggtcattccggtargcagcgatgcagaatcaagacagagtagtttctccttctctctctctctttaattgtaacg

SEQ ID NO: 46 - DNA, 182 bp, Homo sapiens
  allele (1)..(182): rs1333049; wherein s is c or g
tctgcttcatattccaacttgtgtatgacacttcttaggctatcatttcattccaaatttatggtcactaccctactgtcattcctcatactaaccatatgatcaacagttsaaaagcagccactcgcagaggtaagcaagatatatggtaaatactgtgttgacaaaagtatgcagaagca

SEQ ID NO: 47 - DNA, 173 bp, Homo sapiens
  allele (1)..(173): rs2383206; wherein r is g or a
tggcccgatgattttcagttaaccaaattctcccttactatcctggttgccccttctgtcttttccttagaaatgttattgtagtrtttgcaagatggcctgaatcctgaaccccccatcttcaatgagcaccaaatggtaattatagattcccagctgtagagctatgtcag

SEQ ID NO: 48 - DNA, 158 bp, Homo sapiens
  allele (1)..(158): rs2383207; wherein r is g or a
atacttagcccttgggaccattttttactcctgttcggatcccttcrgctaagcatgattatttactattttcagctattagttatgtcttgttgaaaaagtatgaaaagagctgcccaataaattagagtgtatgctcaacattctcttagcttctt

SEQ ID NO: 49 - DNA, 160 bp, Homo sapiens
  allele (1)..(160): rs383830; wherein w is t or a
cctgatgtaaactactctttgttcaacccttagtagtacaaatatgatactttatttttactgttactcatgttgccttgaaaactcctgtgttctgttatctttgaatgtgagctagtwactttattttaatttttggaagtcctgtgggtgtaaattg

SEQ ID NO: 50 - DNA, 214 bp, Homo sapiens
  allele (1)..(214): rs7250581; wherein r is g or a
ctccaaaagccaggagaatgggagggaagtgagggttgaaaaattacctatcaggtagagtgttcactgttcgggaggtgggtttgctagaagctcaatcccaaccattacrctatatgcctatgtaacaaacacacacatatacttaaaatttgttttaaaaacccaaatttctggcttctcctgaaaaaaatataatatgcagccacacggg

SEQ ID NO: 51 - DNA, 187 bp, Homo sapiens
  allele (1)..(187): rs10733113; wherein r is g or a
cacagtctgttacaagggtggaatgaattgtttcttgtaaagcactcagaacaatgagtggcacagagtgatacatgttgagggctttttgttgttgttgttgttgatrtattgtctcagcaccctattatatttttcacatggaggggataaaaaaaatctttcttaagacaggccgcaagaagta

SEQ ID NO: 52 - DNA, 181 bp, Homo sapiens
  allele (1)..(181): rs10761659; wherein r is g or a
actgaaagtgctccttcacaaatgaacacttaaattcaggagcactttcagttaaagcaaaggagttaaagcaaagactttgggagtcagtatcaaataaagatcatctctcaaactrtaacagaaggaaaacaggaattaatttatttcagactttttagaaacgccctcctctttgact

SEQ ID NO: 53 - DNA, 233 bp, Homo sapiens
  allele (1)..(233): rs10883365; wherein r is g or a
gccgcataagacgttacttaaacatgttacttaaacaagactgcagtaaacgtttctttccaagtgagaaaggtctttttcgttctcagacggtttgaaggtrtttgtgccaacgtgacccccggggagatttggaggaagctttctacgtcctaggaggctgagatcccacggagccggtttacggttgagagcagacagtttcgagtagatagcgctggaagagacacgaa

SEQ ID NO: 54 - DNA, 217 bp, Homo sapiens
  allele (1)..(217): rs17234657; wherein k is g or t
agtgctgaagcggaattgagctccttaagttttgtacatcatgtttttttaggttcccactgagctgatttttggccatgattcacacatatctctcctccaaggctcctctcacaaagcatttcctcccagtcacgttktcaaatagcttctcattccctgtatgcctgtgtgtgcatggcctcatctcactttcgctgtgaccattgctgctcat

SEQ ID NO: 55 - DNA, 242 bp, Homo sapiens
  allele (1)..(242): rs55646866; wherein y is c or t
agagtcctcagcctcgtcagttattccttctagtgctggggacgaagggaagaggaggagaaggagctgggacccagcagtgatgggcctatgggagggaggatayggctgcacagccctcagcgcgtggctcaggcagggtcagcccctctgcacatgcctccccctaccaccaccacacgtcatcgcctttttatgtggtctgactttttcagatttttcaacctgaagcttgctttctc

SEQ ID NO: 56 - DNA, 191 bp, Homo sapiens
  allele (1)..(191): rs6672995; wherein r is g or a
agggttcctggctcctacagaagacttgctttaggactgaaggctatattgcagtctgtgttggccttagtcgcggagggacatttaargatggacttactagaaatgctcttcatattccaggaacacacagcacatttcctctgatgggctgctgggaccttaccatttactggaacccaaccctctga

SEQ ID NO: 57 - DNA, 124 bp, Homo sapiens
  allele (1)..(124): ss107635144; wherein y is c or t
actagagtgtgtgattcaggtaaagcatgagacctgaactggcttcaacaccaggctyggtcactcatgccatgtgtctttgagcaggttacttaacctatctgtgcctcacttgtgttttctt

SEQ ID NO: 58 - DNA, 134 bp, Homo sapiens
  allele (1)..(134): rs12037606; wherein r is g or a
tcttagtacatacgttccaaatrtgaatcagctgtgataaagcttgtcaaaacactaacttagtcttagactgggaacagtactaaaataaagggaatgttagatgttgcataccatgaacagctgagctacct

SEQ ID NO: 59 - DNA, 175 bp, Homo sapiens
  allele (1)..(175): rs6601764; wherein y is c or t
atggttttgagctttcagaggtgacaggagtyaagtaagtgagtttatgatgtaagcacacttgaatgctcctttaatctttagagcgggggccactgatctttgttaatttccacaaaatctctgcaaagccgcgttcttcctggattactcagaaaagccttccagatggtga

SEQ ID NO: 60 - DNA, 130 bp, Homo sapiens
  allele (1)..(130): rs7807268; wherein s is g or c
ctctctctctaaatgccttgggaccatcatgtctaacccttcgctacagacattggtgagsacagcttaggccatggtgatgttcatactgtagtgtccaaacaggaggaaatcacccttccagtccctt

SEQ ID NO: 61 - DNA, 174 bp, Homo sapiens
  allele (1)..(174): rs6957669; wherein r is g or a
tggtggtgattactgcccttgctgggggtcacacagatgcatctgggaggatctggaaggggcctgcccctcttgagcttggagctccctcatatgrgttcaccagtgaggacacagtcattgttggttagagactgggactcaagttgtaggctcctttcagtctttgcgtca

SEQ ID NO: 62 - DNA, 52 bp, Homo sapiens
  allele (1)..(52): rs12970134; wherein r is g or a
actgactcttaccaaacaaagcatgarcaaacaaagatttatcagaagggtg

SEQ ID NO: 63 - DNA, 180 bp, Homo sapiens
  allele (1)..(180): rs17782313; wherein y is c or t
cttggaagcaggaaaaccagaatatatgtgagcatctttaatgactacaacattatagaagtttaaagcaggagagattgtatccygatggaaatgacaagaaaagcttcagggggaaggtgacatttaagttggaatattattgaggagtatcattttagcatctgggattgaggtagc

SEQ ID NO: 64 - DNA, 132 bp, Homo sapiens
  allele (1)..(132): rs1859962; wherein k is g or t
tcacaaagaacaccttggaccagttcttgatataaataagaggctgcagacttttccaaatccctgcccgtgkgatgaacactttaaaggtcccaagatttctaataatggggctaaatttcccaaaatgtg

SEQ ID NO: 65 - DNA, 155 bp, Homo sapiens
  allele (1)..(155): rs983085; wherein r is g or a
ggaattgtacaccatcaccaaatatggcatataccaggtatgtgaggctggttcaatatttgaaaaccagtcactgtaatacaccctrttaacaaactaaggatgaaaaatgtacatgatcataacaatcaatggagaaaaagcatttgacaaaa

SEQ ID NO: 66 - DNA, 181 bp, Homo sapiens
  allele (1)..(181): rs10490072; wherein y is c or t
tttgaaatgcaagctccaagagagtgaagccccagcctgcactgccttactttgtgcagagaatgcttctttggttatgtatatacatgcytgcttattctaatccatgcctttattacgaaattcatctaatgttgtggccaaatggcaataaaataatattattacaggacacgggcct

SEQ ID NO: 67 - DNA, 150 bp, Homo sapiens
  allele (1)..(150): rs1153188; wherein w is t or a
gaagatggtctgaatggcaaaatggataaaattaaaatcaaaactagtgaactgaaatagcaaggtgagaagttcttctgaawtgcagtataaaagataaaaagaaatacaaagaaaaagtcatgaaggacagatccagtggacgaaaca

SEQ ID NO: 68 - DNA, 217 bp, Homo sapiens
  allele (1)..(217): rs13071168; wherein r is g or a
cccacatccagacttctgctctgattctcacttccactcaccacacgtacccatctgttcaccaaaatcacactgctgttcacaccagaagtccctcctctacgatcarattcctaatcccaatttctactcacacacctcgtgggaggccaacaccttcttctggttcttcattctcttcctccccagggctgaccatcaccaaagccaaacagct

SEQ ID NO: 69 - DNA, 170 bp, Homo sapiens
  allele (1)..(170): rs17705177; wherein w is t or a
tcagtttccttccccagaaaattgtatatcttgtagggttattgtgaagattaaagtggaatgtgcatgcaaaagtactttgcaaaccacaaagctctaggttggwgtaaataactgaacttttaaaaaaaatttactttaagttctgggatacaacgtgcagaacgtgc

SEQ ID NO: 70 - DNA, 183 bp, Homo sapiens
  allele (1)..(183): rs358806; wherein m is c or a
actttctggagggcagtttggcaatatttgtcaaatttttgaatgtgcgtgggctttgaccgaataactctactcacaaggatatgttctaaaaagaaaaacacacacgtacatgtgcagtacaaacagcaaaactcaatattcaamgttcaataaaattcgtaccactttaaaatgatgagc

SEQ ID NO: 71 - DNA, 172 bp, Homo sapiens
  allele (1)..(172): rs5015480; wherein y is c or t
gctcaccctagggaagtgttcttagggaagcatttctaatatttccagctgtccatatattttcaaacaaataatagggtattgaagtaaactcgaatgttgattataygttttctatcaaattattcaagtattcattcagaaaatatttattgagcacctacaatgtggc

SEQ ID NO: 72 - DNA, 171 bp, Homo sapiens
  allele (1)..(171): rs7020996; wherein y is c or t
cattgtgggggaaagtctgtctttagaaaagaaatgtaaactgggcaagtagtctcatcagttaaatgatttccttgttgacataaggtgaggaaaagaagaayaacttttgggaaaagtaactgtgagaatacaagggaagaagaaaaataaggggttgaacattgagga

SEQ ID NO: 73 - DNA, 203 bp, Homo sapiens
  allele (1)..(203): rs7659604; wherein y is c or t
gcaaatgtgttagggtagagaacattttaatgttattatcctaaaaggaatctttagactgataaaagctatggtatttaactgtcatggctataatggccttagctataacttytgaatctcagtgggaatggtaggggaataactgtattgcacaactggtaacttaccttttctgatatttctccaagagaggctgttca

SEQ ID NO: 74 - DNA, 165 bp, Homo sapiens
  allele (1)..(165): rs2733359; wherein y is c or t
gagggttgtgacggtcaactgtttttgtacacatcttcgattattcytcctgttttcagcctcattctctcgttctaggccatcctaaagtacctgtcatctctacgtctgtggccttctctgggctccactaggcatgtcccctttgcatgtattccaagctgg

SEQ ID NO: 75 - DNA, 151 bp, Homo sapiens
  allele (1)..(151): rs4790797; wherein r is g or a
ggagctctttgcaaactgtgaaattctgtgtactttgagggagaataattgttaatatttattaaacattrtattgtatgatttaaccttcataataatggttttctatacagaaccatttttttattcttgttttagaggctgaagtctt

SEQ ID NO: 76 - DNA, 167 bp, Homo sapiens
  allele (1)..(167): rs7223628; wherein y is c or t
tcatcagggaagaagagagagaaagaaatgaaaataaacacagcttgcagcacatttggcattaacatgagatcagctgctctctgacccayttcctcatagttgtttggtgcctattgtcttagaatcacactgaccctagattacagtttcccttaactgctcca

SEQ ID NO: 77 - DNA, 158 bp, Homo sapiens
  allele (1)..(158): rs8182352; wherein y is c or t
aaccgtgctgtctcagcatattggtctgttcctgcacaaccaaaagctgtaacacttctgctttctctgggttcagcccagcagaaccataatgtggaaatttcaactgggctgcctctgtcyttgggcatatgcctcctcctccgtcaaacacactg

SEQ ID NO: 78 - DNA, 187 bp, Homo sapiens
  allele (1)..(187): rs8182354; wherein y is c or t
tgcaaatgagatttggctgtaaacctctaaactcatctccttctgttccttaccttctaccttgctctttacttcttatcattctaagataaattcccytttagagtttctggtcttgaaattacccttctatttttgctatattgcctgtggtctccctttttaacaccttgtaaggccacatctc

SEQ ID NO: 79 - DNA, 215 bp, Homo sapiens
  allele (1)..(215): rs11761231; wherein y is c or t
aaggcatgcagagcttttgtgttcaaagaattctgtctttttcctccctaaagccattgcatttgtttcaaatctacgtgtgactacatttggagataagtagccyttttcagaccttcttgatttcaaaacacagatttggtctgcacgttctcatgataagacagagaaggagaccatggaaatattttgcctgtctgtaattggcagggctg

SEQ ID NO: 80 - DNA, 193 bp, Homo sapiens
  allele (1)..(193): rs6920220; wherein r is g or a
tgctacggcagcgtaacatagtaggtgaagtacccattgataaattatattttatctgcttccatctgttagcaggtaacttctccactaaaargatatggttctgtagaacaatggcatatgcagacagtgatctgttattccactattctcttaagctatcaatcagattgatgaggcaaatttatgcttc

SEQ ID NO: 81 - DNA, 188 bp, Homo sapiens
  allele (1)..(188): rs6679677; wherein m is c or a
atttttcaggtgccctgttggaaactattcagtgcttcctgcggctaccagcgaacaaggtctgaatccttgctcccaamcaataatctgtgatcttaagcaatttattcaactaacaagcctgttttctcacctgtattatggagatagtcaccttcttaaggatgtgaggattaaatgagaaaccc

SEQ ID NO: 82 - DNA, 179 bp, Homo sapiens
  allele (1)..(179): rs12141187; wherein y is c or t
tcagcatcagtcacctcagccaggtccctgaatcacagccaagcctagatgagtggtattattgaccatgataatgggaggatgaatggtggctatgactgyctgctgcaatcaacctttaggatggccagaaattctgatttggccagcccttggcccagacagcaatgtccccaaga

SEQ ID NO: 83 - DNA, 222 bp, Homo sapiens
  allele (1)..(222): rs4132958; wherein y is c or t
tagacacaggcctgcacaaagagcttgcaatctatagatggatcagttgtcattatataaagctccatatcttcattatcaaaagcagctatgctgaatgcycttctctgaaagattgtaagcaagctctgcagaacctgggcaggccagggtgagccttgctctgtggagattataacagaaaataaaaaataaaggaaatgtagatgggcataccagctc

SEQ ID NO: 84 - DNA, 151 bp, Homo sapiens
  allele (1)..(151): rs952477; wherein r is g or a
gccttcatgccctgacttcagtgggagagaattaggcatggttggtagtggattccctctccttttctcctgtccrtggaggctattgttccaagcccaccacaagagttcttaagcctgggatcccagaagattccatttgccttaagcc

SEQ ID NO: 85 - DNA, 227 bp, Homo sapiens
  allele (1)..(227): rs10798269; wherein r is g or a
tggaccatttgaggtgatgagcctgaccctctaaaaaaaggttaagcaatttaatgggtgaggaagtttttttgaagcctatatccccaaccagttccccagggcagrtagatttgtaaggagaaaaggaggagagattggtcgacctcaagaaatctagatattcttcaggtaacaaacaagaaagcagacacaggtgaatgctttggtttccctggaggtctctc

SEQ ID NO: 86 - DNA, 155 bp, Homo sapiens
  allele (1)..(155): rs729302; wherein m is c or a
tgaagccctgctgagaaagtactgggtccctattggaacccactctctgcacatctggaaatctttggaaatagaccagagaccagggtgcaggtgtgccatgggacaaggtgaagacmcaggatcacctacacaccagagtccacccagtagga

SEQ ID NO: 87 - DNA, 163 bp, Homo sapiens
  allele (1)..(163): rs11171739; wherein y is c or t
ggagggaccaatcaacagtcttataagtagatacaacagtgtataaacaaggaaaccaaggaagatttttctcyttcagaactcggaccctgaataccaggttgagctggagctgagtgagtaataaaatgaaaggccctttaatgtgggggagggtaggtag

SEQ ID NO: 88 - DNA, 224 bp, Homo sapiens
  allele (1)..(224): rs7716600; wherein m is c or a
tgtgaacttgtatggcaaccaaaatgatcaatatatgaagtgaagtaggcataacactaagaagaaactaaaaaacttataatgatagttgagtgtgttaacccatctcttttggaaacagagtagcagacaagaatattataggaagatgtgcacatgtaccmcaaagcttaaagtacaattaaaaaaaaagaatattataggaagatggtgaaaaggaagag

SEQ ID NO: 89 - DNA, 149 bp, Homo sapiens
  allele (1)..(149): rs11249433; wherein y is c or t
ttggaaacatggatccaaaactgtgaaagaaaaagcagagaaagcagggctgggtttaaytttggagttccttggttgcttctccttagcacagtgactcatttgatatcatctttaatttctctggctaaaggttttccaacagatat

SEQ ID NO: 90 - DNA, 227 bp, Homo sapiens
  allele (1)..(227): rs3803662; wherein y is c or t
ttgtcatccaaagcaccaactatgagagatatctatgtgcaatggtatatagatctgtcatagaagggtttaattatatctgcctaatgattttctctccttaatgcctctatagctgtcycttagcgaagaataaaactgtggactgacccccacccatttgcgaagaaagtactgggtcttcagctttcattgttcagccggtggtctttgtggacaacaccagg

SEQ ID NO: 91 - DNA, 237 bp, Homo sapiens
  allele (1)..(237): rs393152; wherein r is g or a
cctactgccttggaatctgctgaagaccaagcccctgcccccaagccatggcaaagaaggagggaaggaagcaaaggtgcccagcggggacaactcggggaggggcgaggtgcccagggcccaggaaggccaagcagcatgtggcagggcagcatcaggtgactcccaagaaggaatgaggagaggatrtgaggaaagagccacagcacagaggcctgctgttaggtcagcggagac

SEQ ID NO: 92 - DNA, 198 bp, Homo sapiens
  allele (1)..(198): rs1491923; wherein y is c or t
tctgcacctttggcttttaggaatcyactttgctctggcattctcctaattttctagaaaattattggtctatttcataattttatcttcatttccttaaatcccaaatattgatatttcccaagggtttatttttgacacttttcccttcttgcttgagatcaatgattcttaattaatgtgtgttgggaaagaggg

SEQ ID NO: 93 - DNA, 214 bp, Homo sapiens
  allele (1)..(214): rs2736098; wherein r is g or a
cgtggtttctgtgtggtgtcacctgccagacccgccgaagaagccacctctttggagggtgcgctctctggcacgcgccactcccacccatccgtgggccgccagcaccacgcrggccccccatccacatcgcggccaccacgtccctgggacacgccttgtcccccggtgtacgccgagaccaagcacttcctctactcctcaggcgacaagg

SEQ ID NO: 94 - DNA, 152 bp, Homo sapiens
  allele (1)..(152): rs801114; wherein k is g or t
ctccccagtgcatcattttcagttttgtcttttactttcaaagaaagctgtctttctgacactgcattctgccctttctgacccakgtcccatatttaaaggcttcacatagactatataatccaagttatccctctgtggagaaagtggct

SEQ ID NO: 95 - DNA, 150 bp, Homo sapiens
  allele (1)..(150): rs2151280; wherein y is c or t
actcgatggccctcaaaagygaaacaagctactatcaggacctctatagaaaaagtttgccaacctctacactgtagtatgccttaaggatttttagaagattgagtatgataaacactttcaaagaatgatgaaattctgagaaatggg

SEQ ID NO: 96 - DNA, 173 bp, Homo sapiens
  allele (1)..(173): rs4636294; wherein r is g or a
gggttgagccagatcttcaagacttaaaaggatttaagtccratagtaaaaggagcgaagggaattctagtaaaagggaacagcttgaggaatgacctagagacatgacagtgatctttggagaaatggcagttagacagacattctgtctactcgtttccctgttacatccc

SEQ ID NO: 97 - DNA, 190 bp, Homo sapiens
  allele (1)..(190): rs823128; wherein r is g or a
actggctttgggttgttcacagtrggatacaaattcctgcttcatctcttaatagttaggtgaactgtgtagttactttttttatcctaacctcaggcctaacatatgaaatgaggataacatatgcctttaagagttgtgcatgattttgaaatatgtataaagtacctggtggaattatttggcatct

SEQ ID NO: 98 - DNA, 223 bp, Homo sapiens
  allele (1)..(223): rs947211; wherein r is g or a
aaaggccagggaaagaagacaggaaaaaagtgaaaactaaagagaaaattttgcttcaragaactggttgtgtggttcccaactgtccatatggcacaggaaagtctcatctgtgaaacaaaataaagttcccttccaacacagacatgactgttctaatttcctatgttatttcaactctctaggaggtgagaaaagcagaaattattgcaccctaggccat

SEQ ID NO: 99 - DNA, 188 bp, Homo sapiens
  allele (1)..(188): rs2736990; wherein y is c or t
atgtctgcctttgcatcagataatggcttacaagttaatctcctcttgctccctgttacacacatatacaycttcttcctaaacagctcataaggtgaaagaaagactcagatttctgactatgtaattgataatatcacacggactgcctgctcatcatctgctagtcacattggcagagttgacag

SEQ ID NO: 100 - DNA, 236 bp, Homo sapiens
  allele (1)..(236): rs12418451; wherein r is g or a
gtaagggagtgctgctcctggacctgctcctgagaatggctcctgggagtgatgtaggtgactgattgatggggtgggacgaagctgggcagaggcttgggtagctgggactgtaacagttatgtgagaggaagcgggaatctgagagagttgccrgggcaaaatgtaggcccccagcccctggttcaggggacagcccagggatagtcaccagggatccagcgatgtgtgtgtgt

SEQ ID NO: 101 - DNA, 205 bp, Homo sapiens
  allele (1)..(205): rs10896449; wherein r is g or a
agcagaatgtggaaggatgggcaggagttgtctaagagaagagtgtggcaatagaagggcaccctgggccacagggaacaaaccatagctgaaagatgaggagtcaagaaatattctggcacccatggggtactattagcagtttaactttacaggagctgaaartttaagaaggggaatgtcaagagatgaggctgaaccttgg

SEQ ID NO: 102 - DNA, 20 bp, Artificial Sequence
  Chemically Synthesized Primer Sequence
ccacgcacaagtgatctacc

SEQ ID NO: 103 - DNA, 22 bp, Artificial Sequence
  Chemically Synthesized Primer Sequence
caagatgcctctatgccttaaa

SEQ ID NO: 104 - DNA, 24 bp, Artificial Sequence
  Chemically Synthesized Primer Sequence
tctttaatacagattgggaagagg

SEQ ID NO: 105 - DNA, 20 bp, Artificial Sequence
  Chemically Synthesized Primer Sequence
aactttcaactgccaggaca

SEQ ID NO: 106 - DNA, 20 bp, Artificial Sequence
  Chemically Synthesized Primer Sequence
acagttgtgcaaccatcagc

SEQ ID NO: 107 - DNA, 21 bp, Artificial Sequence
  Chemically Synthesized Primer Sequence
gactttctggaaaaggcaaaa

SEQ ID NO: 108 - DNA, 20 bp, Artificial Sequence
  Chemically Synthesized Primer Sequence
ttgtgttcaagcctccttcc

SEQ ID NO: 109 - DNA, 20 bp, Artificial Sequence
  Chemically Synthesized Primer Sequence
tctgagcttagcctccctga

SEQ ID NO: 110 - DNA, 20 bp, Artificial Sequence
  Chemically Synthesized Primer Sequence
ggaaaacactgggagggaat

SEQ ID NO: 111 - DNA, 20 bp, Artificial Sequence
  Chemically Synthesized Primer Sequence
cctgggtgacagaggaagac

SEQ ID NO: 112 - DNA, 20 bp, Artificial Sequence
  Chemically Synthesized Primer Sequence
ggttctgtgaagcgggtaaa

SEQ ID NO: 113 - DNA, 20 bp, Artificial Sequence
  Chemically Synthesized Primer Sequence
tcatggaccacaaatttcca

SEQ ID NO: 114 - DNA, 20 bp, Artificial Sequence
  Chemically Synthesized Primer Sequence
gtggggtctttgttgtggag

SEQ ID NO: 115 - DNA, 20 bp, Artificial Sequence
  Chemically Synthesized Primer Sequence
tgttcagagcggttgaatga

SEQ ID NO: 116 - DNA, 20 bp, Artificial Sequence
  Chemically Synthesized Primer Sequence
gccatgtctaactgggcatt

SEQ ID NO: 117 - DNA, 20 bp, Artificial Sequence
  Chemically Synthesized Primer Sequence
gctgagtgatgctggcaata

SEQ ID NO: 118 - DNA, 24 bp, Artificial Sequence
  Chemically Synthesized Primer Sequence
gacacgtggaatttactcttttga

SEQ ID NO: 119 - DNA, 21 bp, Artificial Sequence
  Chemically Synthesized Primer Sequence
gccaagcctacacttcctctt

SEQ ID NO: 120 - DNA, 20 bp, Artificial Sequence
  Chemically Synthesized Primer Sequence
ccgtgggaaacaaagtcttc

SEQ ID NO: 121 - DNA, 21 bp, Artificial Sequence
  Chemically Synthesized Primer Sequence
ttccaacctgaaacacacaca

SEQ ID NO: 122 - DNA, 20 bp, Artificial Sequence
  Chemically Synthesized Primer Sequence
ctccatagagcctgcagagg

SEQ ID NO: 123 - DNA, 23 bp, Artificial Sequence
  Chemically Synthesized Primer Sequence
ttattcgtccctctgttttatgg

SEQ ID NO: 124 - DNA, 20 bp, Artificial Sequence
  Chemically Synthesized Primer Sequence
tcctttgagctcagcagatg

SEQ ID NO: 125 - DNA, 20 bp, Artificial Sequence
  Chemically Synthesized Primer Sequence
tgagaaacttgctgggttcc

SEQ ID NO: 126 - DNA, 20 bp, Artificial Sequence
  Chemically Synthesized Primer Sequence
gcttgcagcttctgcctaat

SEQ ID NO: 127 - DNA, 20 bp, Artificial Sequence
  Chemically Synthesized Primer Sequence
aacttttggggaggctgttt

SEQ ID NO: 128 - DNA, 20 bp, Artificial Sequence
  Chemically Synthesized Primer Sequence
aggctccttagggaaggtga

SEQ ID NO: 129 - DNA, 20 bp, Artificial Sequence
  Chemically Synthesized Primer Sequence
gagattgtgccactgcactc

SEQ ID NO: 130 - DNA, 20 bp, Artificial Sequence
  Chemically Synthesized Primer Sequence
gagttgcacgccagacacta

SEQ ID NO: 131 - DNA, 20 bp, Artificial Sequence
  Chemically Synthesized Primer Sequence
aggggttcctgttgcttttt

SEQ ID NO: 132 - DNA, 22 bp, Artificial Sequence
  Chemically Synthesized Primer Sequence
agtgattgctctaattgccaag

SEQ ID NO: 133 - DNA, 20 bp, Artificial Sequence
  Chemically Synthesized Primer Sequence
gcgcatgaggtctatgttga

SEQ ID NO: 134 - DNA, 20 bp, Artificial Sequence
  Chemically Synthesized Primer Sequence
ggccatctgttttaccaacc

SEQ ID NO: 135 - DNA, 20 bp, Artificial Sequence
  Chemically Synthesized Primer Sequence
tgggaaggagtcgttgagtt

SEQ ID NO: 136 - DNA, 27 bp, Artificial Sequence
  Chemically Synthesized Primer Sequence
cgggaaagtaaaaattgttatctcatt

SEQ ID NO: 137 - DNA, 20 bp, Artificial Sequence
  Chemically Synthesized Primer Sequence
gaggaccaatcctttggaca

SEQ ID NO: 138 - DNA, 21 bp, Artificial Sequence
  Chemically Synthesized Primer Sequence
aaagaggtaacccagggaaca

SEQ ID NO: 139 - DNA, 20 bp, Artificial Sequence
  Chemically Synthesized Primer Sequence
cataagccttcgctgactcc

SEQ ID NO: 140 - DNA, 20 bp, Artificial Sequence
  Chemically Synthesized Primer Sequence
tgagccaggacatcagaaag

SEQ ID NO: 141 - DNA, 20 bp, Artificial Sequence
  Chemically Synthesized Primer Sequence
ccatccctgtgagtcatcct

SEQ ID NO: 142 - DNA, 23 bp, Artificial Sequence
  Chemically Synthesized Primer Sequence
ttctctccagattgatacacagc

SEQ ID NO: 143 - DNA, 20 bp, Artificial Sequence
  Chemically Synthesized Primer Sequence
aatgcctctgccaataccac

SEQ ID NO: 144 - DNA, 22 bp, Artificial Sequence
  Chemically Synthesized Primer Sequence
tcacaggaaaattgagcagaaa

SEQ ID NO: 145 - DNA, 20 bp, Artificial Sequence
  Chemically Synthesized Primer Sequence
atgtgcaatgccaagaatga

SEQ ID NO: 146 - DNA, 19 bp, Artificial Sequence
  Chemically Synthesized Primer Sequence
gtagccccacttcgccaat

SEQ ID NO: 147 - DNA, 20 bp, Artificial Sequence
  Chemically Synthesized Primer Sequence
tccctatcgcagaggaaaaa

SEQ ID NO: 148 - DNA, 20 bp, Artificial Sequence
  Chemically Synthesized Primer Sequence
aacggtcagacgcaaacagt

SEQ ID NO: 149 - DNA, 20 bp, Artificial Sequence
  Chemically Synthesized Primer Sequence
ggcagctcctcattcctaaa

SEQ ID NO: 150 - DNA, 23 bp, Artificial Sequence
  Chemically Synthesized Primer Sequence
gaccaaattgaagaattggtttg

SEQ ID NO: 151 - DNA, 20 bp, Artificial Sequence
  Chemically Synthesized Primer Sequence
acttgagctcgatccacagc

SEQ ID NO: 152 - DNA, 20 bp, Artificial Sequence
  Chemically Synthesized Primer Sequence
tcaatccccatatgcacaga

SEQ ID NO: 153 - DNA, 20 bp, Artificial Sequence
  Chemically Synthesized Primer Sequence
atgacatgctctcacgatgg

SEQ ID NO: 154 - DNA, 22 bp, Artificial Sequence
  Chemically Synthesized Primer Sequence
tggcagtccaagctactaagaa

SEQ ID NO: 155 - DNA, 20 bp, Artificial Sequence
  Chemically Synthesized Primer Sequence
tgctgcatggtaaatttttg

SEQ ID NO: 156 - DNA, 20 bp, Artificial Sequence
  Chemically Synthesized Primer Sequence
gggaaacgaaggatgaaaga

SEQ ID NO: 157 - DNA, 19 bp, Artificial Sequence
  Chemically Synthesized Primer Sequence
ttcctggttgatttccctt

SEQ ID NO: 158 - DNA, 20 bp, Artificial Sequence
  Chemically Synthesized Primer Sequence
ccatgagcctatcacactcg

SEQ ID NO: 159 - DNA, 20 bp, Artificial Sequence
  Chemically Synthesized Primer Sequence
tgccgatatttccgattttc

SEQ ID NO: 160 - DNA, 23 bp, Artificial Sequence
  Chemically Synthesized Primer Sequence
atgttcatcagtggtcacaaata

SEQ ID NO: 161 - DNA, 20 bp, Artificial Sequence
  Chemically Synthesized Primer Sequence
ggctcatggtaggtcgtcat

SEQ ID NO: 162 - DNA, 20 bp, Artificial Sequence
  Chemically Synthesized Primer Sequence
agctctgactcccaactcca

SEQ ID NO: 163 - DNA, 20 bp, Artificial Sequence
  Chemically Synthesized Primer Sequence
cgacagatggctacaaagca

SEQ ID NO: 164 - DNA, 20 bp, Artificial Sequence
  Chemically Synthesized Primer Sequence
gctcactacctgggtgcaat

SEQ ID NO: 165 - DNA, 22 bp, Artificial Sequence
  Chemically Synthesized Primer Sequence
ttgtcagcatttttcagatttc

SEQ ID NO: 166 - DNA, 21 bp, Artificial Sequence
  Chemically Synthesized Primer Sequence
tgtgcacaagagcattgtttt

SEQ ID NO: 167 - DNA, 20 bp, Artificial Sequence
  Chemically Synthesized Primer Sequence
ccaggatcatccatgttgtg

SEQ ID NO: 168 - DNA, 20 bp, Artificial Sequence
  Chemically Synthesized Primer Sequence
gagtgatggagtggcatagg

SEQ ID NO: 169 - DNA, 20 bp, Artificial Sequence
  Chemically Synthesized Primer Sequence
aaccccctacatgcttcctt

SEQ ID NO: 170 - DNA, 20 bp, Artificial Sequence
  Chemically Synthesized Primer Sequence
ccctgttttgttgcagtgaa

SEQ ID NO: 171 - DNA, 20 bp, Artificial Sequence
  Chemically Synthesized Primer Sequence
acggagcactctcaacatcc

SEQ ID NO: 172 - DNA, 20 bp, Artificial Sequence
  Chemically Synthesized Primer Sequence
cagaagcagcagcaggtaca

SEQ ID NO: 173 - DNA, 20 bp, Artificial Sequence
  Chemically Synthesized Primer Sequence
aagctactggcccaaagaca

SEQ ID NO: 174 - DNA, 22 bp, Artificial Sequence
  Chemically Synthesized Primer Sequence
tatcgacaaaagttttccactg

SEQ ID NO: 175 - DNA, 20 bp, Artificial Sequence
  Chemically Synthesized Primer Sequence
ccctgctaacactgctggac

SEQ ID NO: 176 - DNA, 19 bp, Artificial Sequence
  Chemically Synthesized Primer Sequence
ggcattgcgttcattctga

SEQ ID NO: 177 - DNA, 20 bp, Artificial Sequence
  Chemically Synthesized Primer Sequence
agcgagactccgtctgaaaa

SEQ ID NO: 178 - DNA, 21 bp, Artificial Sequence
  Chemically Synthesized Primer Sequence
agctgctataagatgggctga

SEQ ID NO: 179 - DNA, 20 bp, Artificial Sequence
  Chemically Synthesized Primer Sequence
tgccactgctaaaagccatt

SEQ ID NO: 180 - DNA, 20 bp, Artificial Sequence
  Chemically Synthesized Primer Sequence
gtttctgcacatggtgatgg

SEQ ID NO: 181 - DNA, 20 bp, Artificial Sequence
  Chemically Synthesized Primer Sequence
ctgcctcactctccagttcc

SEQ ID NO: 182 - DNA, 20 bp, Artificial Sequence
  Chemically Synthesized Primer Sequence
caaacagccaatttgtggag

SEQ ID NO: 183 - DNA, 25 bp, Artificial Sequence
  Chemically Synthesized Primer Sequence
ggcgttacaattaaagagagagaga

SEQ ID NO: 184 - DNA, 23 bp, Artificial Sequence
  Chemically Synthesized Primer Sequence
tctgcttcatattccaacttgtg

SEQ ID NO: 185 - DNA, 23 bp, Artificial Sequence
  Chemically Synthesized Primer Sequence
tgcttctgcatacttttgtcaac

SEQ ID NO: 186 - DNA, 20 bp, Artificial Sequence
  Chemically Synthesized Primer Sequence
ggcccgatgattttcagtta

SEQ ID NO: 187 - DNA, 23 bp, Artificial Sequence
  Chemically Synthesized Primer Sequence
gacatagctctacagctgggaat

SEQ ID NO: 188 - DNA, 20 bp, Artificial Sequence
  Chemically Synthesized Primer Sequence
acttagcccttgggaccatt

SEQ ID NO: 189 - DNA, 24 bp, Artificial Sequence
  Chemically Synthesized Primer Sequence
aagaagctaagagaatgttgagca

SEQ ID NO: 190 - DNA, 25 bp, Artificial Sequence
  Chemically Synthesized Primer Sequence
gacccctgatgtaaactactctttg

SEQ ID NO: 191 - DNA, 20 bp, Artificial Sequence
  Chemically Synthesized Primer Sequence
gctggtgggtttctgtagga

SEQ ID NO: 192 - DNA, 20 bp, Artificial Sequence
  Chemically Synthesized Primer Sequence
ctccaaaagccaggagaatg

SEQ ID NO: 193 - DNA, 19 bp, Artificial Sequence
  Chemically Synthesized Primer Sequence
cccgtgtggctgcatatta

SEQ ID NO: 194 - DNA, 22 bp, Artificial Sequence
  Chemically Synthesized Primer Sequence
cacagtctgttacaagggtgga

SEQ ID NO: 195 - DNA, 20 bp, Artificial Sequence
  Chemically Synthesized Primer Sequence
tacttcttgcggcctgtctt

SEQ ID NO: 196 - DNA, 20 bp, Artificial Sequence
  Chemically Synthesized Primer Sequence
ggattcttcgcatgatgagg

SEQ ID NO: 197 - DNA, 20 bp, Artificial Sequence
  Chemically Synthesized Primer Sequence
agtcaaagaggagggcgttt

SEQ ID NO: 198 - DNA, 20 bp, Artificial Sequence
  Chemically Synthesized Primer Sequence
gaaggccgcataagacgtta

SEQ ID NO: 199 - DNA, 20 bp, Artificial Sequence
  Chemically Synthesized Primer Sequence
cgtgtctcttccagcgctat

SEQ ID NO: 200 - DNA, 20 bp, Artificial Sequence
  Chemically Synthesized Primer Sequence
agtgctgaagcggaattgag

SEQ ID NO: 201 - DNA, 20 bp, Artificial Sequence
  Chemically Synthesized Primer Sequence
atgagcagcaatggtcacag

SEQ ID NO: 202 - DNA, 20 bp, Artificial Sequence
  Chemically Synthesized Primer Sequence
agagtcctcagcctcgtcag

SEQ ID NO: 203 - DNA, 20 bp, Artificial Sequence
  Chemically Synthesized Primer Sequence
cgagaaagcaagcttcaggt

SEQ ID NO: 204 - DNA, 20 bp, Artificial Sequence
  Chemically Synthesized Primer Sequence
agggttcctggctcctacag

SEQ ID NO: 205 - DNA, 20 bp, Artificial Sequence
  Chemically Synthesized Primer Sequence
cagagggttgggttccagta

SEQ ID NO: 206 - DNA, 20 bp, Artificial Sequence
  Chemically Synthesized Primer Sequence
gcgtggtgaggtgattactg

SEQ ID NO: 207 - DNA, 22 bp, Artificial Sequence
  Chemically Synthesized Primer Sequence
aagaaaacacaagtgaggcaca

SEQ ID NO: 208 - DNA, 20 bp, Artificial Sequence
  Chemically Synthesized Primer Sequence
ctggcagaggatttgagaca

SEQ ID NO: 209 - DNA, 21 bp, Artificial Sequence
  Chemically Synthesized Primer Sequence
aggtagctcagctgttcatgg

SEQ ID NO: 210 - DNA, 20 bp, Artificial Sequence
  Chemically Synthesized Primer Sequence
accagtggtccaacccacta

SEQ ID NO: 211 - DNA, 20 bp, Artificial Sequence
  Chemically Synthesized Primer Sequence
tcaccatctggaaggctttt

SEQ ID NO: 212 - DNA, 20 bp, Artificial Sequence
  Chemically Synthesized Primer Sequence
ggaggacaggttggagaaca

SEQ ID NO: 213 - DNA, 20 bp, Artificial Sequence
  Chemically Synthesized Primer Sequence
aagggactggaagggtgatt

SEQ ID NO: 214 - DNA, 20 bp, Artificial Sequence
  Chemically Synthesized Primer Sequence
ctaggcgtttgcattcatcc

SEQ ID NO: 215 - DNA, 20 bp, Artificial Sequence
  Chemically Synthesized Primer Sequence
tgacgcaaagactgaaagga

SEQ ID NO: 216 - DNA, 20 bp, Artificial Sequence
  Chemically Synthesized Primer Sequence
ggtggtgattactgcccttg

SEQ ID NO: 217 - DNA, 20 bp, Artificial Sequence
  Chemically Synthesized Primer Sequence
cagtgtggagacatgcttgc

SEQ ID NO: 218 - DNA, 20 bp, Artificial Sequence
  Chemically Synthesized Primer Sequence
cttggaagcaggaaaaccag

SEQ ID NO: 219 - DNA, 20 bp, Artificial Sequence
  Chemically Synthesized Primer Sequence
gctacctcaatcccagatgc

SEQ ID NO: 220 - DNA, 20 bp, Artificial Sequence
  Chemically Synthesized Primer Sequence
cccggaaggcaaataacaat

SEQ ID NO: 221 - DNA, 20 bp, Artificial Sequence
  Chemically Synthesized Primer Sequence
ttgggaaatttagccccatt

SEQ ID NO: 222 - DNA, 22 bp, Artificial Sequence
  Chemically Synthesized Primer Sequence
ggaattgtacaccatcaccaaa

SEQ ID NO: 223 - DNA, 22 bp, Artificial Sequence
  Chemically Synthesized Primer Sequence
tttgtcaaatgctttttctcca

SEQ ID NO: 224 - DNA, 20 bp, Artificial Sequence
  Chemically Synthesized Primer Sequence
tgcaagctccaagagagtga

SEQ ID NO: 225 - DNA, 20 bp, Artificial Sequence
  Chemically Synthesized Primer Sequence
aggcccgtgtcctgtaataa

SEQ ID NO: 226 - DNA, 21 bp, Artificial Sequence
  Chemically Synthesized Primer Sequence
gaagatggtctgaatggcaaa

SEQ ID NO: 227 - DNA, 20 bp, Artificial Sequence
  Chemically Synthesized Primer Sequence
tgtttcgtccactggatctg

SEQ ID NO: 228 - DNA, 20 bp, Artificial Sequence
  Chemically Synthesized Primer Sequence
cccacatccagacttctgct

SEQ ID NO: 229 - DNA, 20 bp, Artificial Sequence
  Chemically Synthesized Primer Sequence
agctgtttggctttggtgat

SEQ ID NO: 230 - DNA, 20 bp, Artificial Sequence
  Chemically Synthesized Primer Sequence
attaggggccaggaaagaaa

SEQ ID NO: 231 - DNA, 20 bp, Artificial Sequence
  Chemically Synthesized Primer Sequence
tgcctggcatttaaaaatct

SEQ ID NO: 232 - DNA, 20 bp, Artificial Sequence
  Chemically Synthesized Primer Sequence
tcagtttccttccccagaaa

SEQ ID NO: 233 - DNA, 20 bp, Artificial Sequence
  Chemically Synthesized Primer Sequence
gcacgttctgcacgttgtat

SEQ ID NO: 234 - DNA, 20 bp, Artificial Sequence
  Chemically Synthesized Primer Sequence
actttctggagggcagtttg

SEQ ID NO: 235 - DNA, 25 bp, Artificial Sequence
  Chemically Synthesized Primer Sequence
gctcatcattttaaagtggtacgaa

SEQ ID NO: 236 - DNA, 21 bp, Artificial Sequence
  Chemically Synthesized Primer Sequence
gctcaccctagggaagtgttc

SEQ ID NO: 237 - DNA, 20 bp, Artificial Sequence
  Chemically Synthesized Primer Sequence
gccacattgtaggtgctcaa

SEQ ID NO: 238 - DNA, 20 bp, Artificial Sequence
  Chemically Synthesized Primer Sequence
cattgtgggggaaagtctgt

SEQ ID NO: 239 - DNA, 21 bp, Artificial Sequence
  Chemically Synthesized Primer Sequence
tcctcaatgttcaacccctta

SEQ ID NO: 240 - DNA, 24 bp, Artificial Sequence
  Chemically Synthesized Primer Sequence
gcaaatgtgttagggtagagaaca

SEQ ID NO: 241 - DNA, 21 bp, Artificial Sequence
  Chemically Synthesized Primer Sequence
tgaacagcctctcttggagaa

SEQ ID NO: 242 - DNA, 20 bp, Artificial Sequence
  Chemically Synthesized Primer Sequence
cgaaccagagggcataagag

SEQ ID NO: 243 - DNA, 20 bp, Artificial Sequence
  Chemically Synthesized Primer Sequence
caagatcatgggcttcacaa

SEQ ID NO: 244 - DNA, 20 bp, Artificial Sequence
  Chemically Synthesized Primer Sequence
gagggttgtgacggtcaact

SEQ ID NO: 245 - DNA, 20 bp, Artificial Sequence
  Chemically Synthesized Primer Sequence
ccagcttggaatacatgcaa

SEQ ID NO: 246 - DNA, 20 bp, Artificial Sequence
  Chemically Synthesized Primer Sequence
gaagaatttgggcagtgagc

SEQ ID NO: 247 - DNA, 20 bp, Artificial Sequence
  Chemically Synthesized Primer Sequence
atccatggccattcattcat

SEQ ID NO: 248 - DNA, 19 bp, Artificial Sequence
  Chemically Synthesized Primer Sequence
ggcaaggaggcagaacagt

SEQ ID NO: 249 - DNA, 20 bp, Artificial Sequence
  Chemically Synthesized Primer Sequence
gggggaaatgaattgtcaaa

SEQ ID NO: 250 - DNA, 20 bp, Artificial Sequence
  Chemically Synthesized Primer Sequence
aggtggtgatggttttgtcc

SEQ ID NO: 251 - DNA, 25 bp, Artificial Sequence
  Chemically Synthesized Primer Sequence
aagacttcagcctctaaaacaagaa

SEQ ID NO: 252 - DNA, 20 bp, Artificial Sequence
  Chemically Synthesized Primer Sequence
ggagctctttgcaaactgtg

SEQ ID NO: 253 - DNA, 25 bp, Artificial Sequence
  Chemically Synthesized Primer Sequence
aagacttcagcctctaaaacaagaa

SEQ ID NO: 254 - DNA, 23 bp, Artificial Sequence
  Chemically Synthesized Primer Sequence
tcatcagggaagaagagagagaa

SEQ ID NO: 255 - DNA, 22 bp, Artificial Sequence
  Chemically Synthesized Primer Sequence
tggagcagttaagggaaactgt

SEQ ID NO: 256 - DNA, 20 bp, Artificial Sequence
  Chemically Synthesized Primer Sequence
aaccgtgctgtctcagcata

SEQ ID NO: 257 - DNA, 20 bp, Artificial Sequence
  Chemically Synthesized Primer Sequence
cagtgtgtttgacggaggag

SEQ ID NO: 258 - DNA, 20 bp, Artificial Sequence
  Chemically Synthesized Primer Sequence
tgcaaatgagatttggctgt

SEQ ID NO: 259 - DNA, 21 bp, Artificial Sequence
  Chemically Synthesized Primer Sequence
gagatgtggccttacaaggtg

SEQ ID NO: 260 - DNA, 20 bp, Artificial Sequence
  Chemically Synthesized Primer Sequence
tccactcaactccctcaacc

SEQ ID NO: 261 - DNA, 20 bp, Artificial Sequence
  Chemically Synthesized Primer Sequence
agccaagttcttggatctgc

SEQ ID NO: 262 - DNA, 20 bp, Artificial Sequence
  Chemically Synthesized Primer Sequence
aaggcatgcagagcttttgt

SEQ ID NO: 263 - DNA, 20 bp, Artificial Sequence
  Chemically Synthesized Primer Sequence
cagccctgccaattacagac

SEQ ID NO: 264 - DNA, 25 bp, Artificial Sequence
  Chemically Synthesized Primer Sequence
tttgttgatatcttcttgtttggta

SEQ ID NO: 265 - DNA, 23 bp, Artificial Sequence
  Chemically Synthesized Primer Sequence
catggggagagaaaatactctga

SEQ ID NO: 266 - DNA, 20 bp, Artificial Sequence
  Chemically Synthesized Primer Sequence
tgttgctgagaccctcagtg

SEQ ID NO: 267 - DNA, 20 bp, Artificial Sequence
  Chemically Synthesized Primer Sequence
agtcaagcagtagcccagga

SEQ ID NO: 268 - DNA, 20 bp, Artificial Sequence
  Chemically Synthesized Primer Sequence
tgctacggcagcgtaacata

SEQ ID NO: 269 - DNA, 22 bp, Artificial Sequence
  Chemically Synthesized Primer Sequence
gaagcataaatttgcctcatca

SEQ ID NO: 270 - DNA, 20 bp, Artificial Sequence
  Chemically Synthesized Primer Sequence
gcctcctgtgctttctcact

SEQ ID NO: 271 - DNA, 20 bp, Artificial Sequence
  Chemically Synthesized Primer Sequence
gcctcagagagaatcggatg

SEQ ID NO: 272 - DNA, 20 bp, Artificial Sequence
  Chemically Synthesized Primer Sequence
atttttcaggtgccctgttg

SEQ ID NO: 273 - DNA, 23 bp, Artificial Sequence
  Chemically Synthesized Primer Sequence
gggtttctcatttaatcctcaca

SEQ ID NO: 274 - DNA, 20 bp, Artificial Sequence
  Chemically Synthesized Primer Sequence
tcagcatcagtcacctcagc

SEQ ID NO: 275 - DNA, 17 bp, Artificial Sequence
  Chemically Synthesized Primer Sequence
tcttggggacattgctg

SEQ ID NO: 276 - DNA, 20 bp, Artificial Sequence
  Chemically Synthesized Primer Sequence
aatctgggcatagccaacag

SEQ ID NO: 277 - DNA, 20 bp, Artificial Sequence
  Chemically Synthesized Primer Sequence
aggcaaggagggttgttctt

SEQ ID NO: 278 - DNA, 20 bp, Artificial Sequence
  Chemically Synthesized Primer Sequence
tagacacaggcctgcacaaa

SEQ ID NO: 279 - DNA, 21 bp, Artificial Sequence
  Chemically Synthesized Primer Sequence
gagctggtatgcccatctaca

SEQ ID NO: 280 - DNA, 22 bp, Artificial Sequence
  Chemically Synthesized Primer Sequence
tttttaatgccccatgaatatg

SEQ ID NO: 281 - DNA, 20 bp, Artificial Sequence
  Chemically Synthesized Primer Sequence
ggtttctgaggttgcacaca

SEQ ID NO: 282 - DNA, 20 bp, Artificial Sequence
  Chemically Synthesized Primer Sequence
ccagagtggaatcagcaggt

SEQ ID NO: 283 - DNA, 20 bp, Artificial Sequence
  Chemically Synthesized Primer Sequence
cggcgcagactttcttttat

SEQ ID NO: 284 - DNA, 20 bp, Artificial Sequence
  Chemically Synthesized Primer Sequence
tgcataagccaattccttgc

SEQ ID NO: 285 - DNA, 20 bp, Artificial Sequence
  Chemically Synthesized Primer Sequence
aaatcgtttgcttgggtgag

SEQ ID NO: 286 - DNA, 19 bp, Artificial Sequence
  Chemically Synthesized Primer Sequence
gccttcatgccctgacttc

SEQ ID NO: 287 - DNA, 20 bp, Artificial Sequence
  Chemically Synthesized Primer Sequence
ggcttaaggcaaatggaatc

SEQ ID NO: 288 - DNA, 20 bp, Artificial Sequence
  Chemically Synthesized Primer Sequence
tggaccatttgaggtgatga

SEQ ID NO: 289 - DNA, 20 bp, Artificial Sequence
  Chemically Synthesized Primer Sequence
gagagacctccagggaaacc

SEQ ID NO: 290 - DNA, 20 bp, Artificial Sequence
  Chemically Synthesized Primer Sequence
aggttgcagtgagccaagat

SEQ ID NO: 291 - DNA, 20 bp, Artificial Sequence
  Chemically Synthesized Primer Sequence
aatacgtaagcgtggggttg

SEQ ID NO: 292 - DNA, 20 bp, Artificial Sequence
  Chemically Synthesized Primer Sequence
tgaagccctgctgagaaagt

SEQ ID NO: 293 - DNA, 20 bp, Artificial Sequence
  Chemically Synthesized Primer Sequence
tcctactgggtggactctgg

SEQ ID NO: 294 - DNA, 20 bp, Artificial Sequence
  Chemically Synthesized Primer Sequence
ggagggaccaatcaacagtc

SEQ ID NO: 295 - DNA, 20 bp, Artificial Sequence
  Chemically Synthesized Primer Sequence
ctacctaccctcccccacat

SEQ ID NO: 296 - DNA, 21 bp, Artificial Sequence
  Chemically Synthesized Primer Sequence
tcccttaaggcataagacagc

SEQ ID NO: 297 - DNA, 20 bp, Artificial Sequence
  Chemically Synthesized Primer Sequence
tgaggctgcagtgagctatg

SEQ ID NO: 298 - DNA, 21 bp, Artificial Sequence
  Chemically Synthesized Primer Sequence
tgtgaacttgtatggcaacca

SEQ ID NO: 299 - DNA, 21 bp, Artificial Sequence
  Chemically Synthesized Primer Sequence
tcttccttttcaccatcttcc

SEQ ID NO: 300 - DNA, 21 bp, Artificial Sequence
  Chemically Synthesized Primer Sequence
ttggaaacatggaatccaaaa

SEQ ID NO: 301 - DNA, 24 bp, Artificial Sequence
  Chemically Synthesized Primer Sequence
atatctgttggaaaacctttagcc

SEQ ID NO: 302 - DNA, 20 bp, Artificial Sequence
  Chemically Synthesized Primer Sequence
ttgtcatccaaagcaccaac

SEQ ID NO: 303 - DNA, 20 bp, Artificial Sequence
  Chemically Synthesized Primer Sequence
cctggtgttgtccacaaaga

SEQ ID NO: 304 - DNA, 20 bp, Artificial Sequence
  Chemically Synthesized Primer Sequence
cctactgccttggaatctgc

SEQ ID NO: 305 - DNA, 20 bp, Artificial Sequence
  Chemically Synthesized Primer Sequence
gtctccgctgacctaacagc

SEQ ID NO: 306 - DNA, 20 bp, Artificial Sequence
  Chemically Synthesized Primer Sequence
ctgcacctttggcttttagg

SEQ ID NO: 307 - DNA, 20 bp, Artificial Sequence
  Chemically Synthesized Primer Sequence
ccctctttcccaacacacat

SEQ ID NO: 308 - DNA, 20 bp, Artificial Sequence
  Chemically Synthesized Primer Sequence
cgtggtttctgtgtggtgtc

SEQ ID NO: 309 - DNA, 20 bp, Artificial Sequence
  Chemically Synthesized Primer Sequence
ccttgtcgcctgaggagtag

SEQ ID NO: 310 - DNA, 20 bp, Artificial Sequence
  Chemically Synthesized Primer Sequence
ctccccagtgcatcattttc

SEQ ID NO: 311 - DNA, 20 bp, Artificial Sequence
  Chemically Synthesized Primer Sequence
agccactttctccacagagg

SEQ ID NO: 312 - DNA, 19 bp, Artificial Sequence
  Chemically Synthesized Primer Sequence
actcgatggccctcaaaag

SEQ ID NO: 313 - DNA, 22 bp, Artificial Sequence
  Chemically Synthesized Primer Sequence
cccatttctcagaatttcatca

SEQ ID NO: 314 - DNA, 20 bp, Artificial Sequence
  Chemically Synthesized Primer Sequence
gggttgagccagatcttcaa

SEQ ID NO: 315 - DNA, 20 bp, Artificial Sequence
  Chemically Synthesized Primer Sequence
gggatgtaacagggaaacga

SEQ ID NO: 316 - DNA, 20 bp, Artificial Sequence
  Chemically Synthesized Primer Sequence
actggctttgggttgttcac

SEQ ID NO: 317 - DNA, 21 bp, Artificial Sequence
  Chemically Synthesized Primer Sequence
agatgccaaataattccacca

SEQ ID NO: 318 - DNA, 20 bp, Artificial Sequence
  Chemically Synthesized Primer Sequence
aaaggccagggaaagaagac

SEQ ID NO: 319 - DNA, 20 bp, Artificial Sequence
  Chemically Synthesized Primer Sequence
atggcctatgggtgcaataa

SEQ ID NO: 320 - DNA, 20 bp, Artificial Sequence
  Chemically Synthesized Primer Sequence
atgtctgcctttgcatcaga

SEQ ID NO: 321 - DNA, 21 bp, Artificial Sequence
  Chemically Synthesized Primer Sequence
ctgtcaactctgccaatgtga

SEQ ID NO: 322 - DNA, 20 bp, Artificial Sequence
  Chemically Synthesized Primer Sequence
gtaagggagtgctgctcctg

SEQ ID NO: 323 - DNA, 20 bp, Artificial Sequence
  Chemically Synthesized Primer Sequence
acacacacacatcgctggat

SEQ ID NO: 324 - DNA, 20 bp, Artificial Sequence
  Chemically Synthesized Primer Sequence
agcagaatgtggaaggatgg

SEQ ID NO: 325 - DNA, 20 bp, Artificial Sequence
  Chemically Synthesized Primer Sequence
ccaaggttcagcctcatctc

SEQ ID NO: 326 - DNA, 21 bp, Artificial Sequence
  Chemically Synthesized Primer Sequence
cacgcacaagtgatctaccag

SEQ ID NO: 327 - DNA, 19 bp, Artificial Sequence
  Chemically Synthesized Primer Sequence
gcatcaggatgcaccagtc

SEQ ID NO: 328 - DNA, 20 bp, Artificial Sequence
  Chemically Synthesized Primer Sequence
ccacgcacaagtgatctacc

SEQ ID NO: 329 - DNA, 20 bp, Artificial Sequence
  Chemically Synthesized Primer Sequence
tccccttacatctgccactt

SEQ ID NO: 330 - DNA, 20 bp, Artificial Sequence
  Chemically Synthesized Primer Sequence
gtgttcaggagctgggtgac

SEQ ID NO: 331 - DNA, 20 bp, Artificial Sequence
  Chemically Synthesized Primer Sequence
tccccttacatctgccactt

SEQ ID NO: 332 - DNA, 152 bp, Homo sapiens
  allele (1)..(152): 152 nucleotide corresponding to rs2670660; wherein r is g or a
ccacgcacaagtgatctaccagtcttttaaarttctattattaaaacccaaacatgctctttcatttccacagaacactgggtctaaatttagactggtgcatcctgatgctgcaccagtctgctcttaatttaaggcatacaggcatcttg

SEQ ID NO: 333 - DNA, 110 bp, Homo sapiens
  allele (1)..(110): 110 nucleotide corresponding to rs2670660; wherein r is g or a
cacgcacaagtgatctaccagtcttttaaarttctattattaaaacccaaacatgctctttcatttccacagaacactgggtctaaatttagactggtgcatcctgatgc